(19) United States
(12) Patent Application Publication
(10) Pub. No.: US 2023/0089368 A1
(43) Pub. Date: Mar. 23, 2023

(54) POLYAROMATIC UREA DERIVATIVES AND THEIR USE IN THE TREATMENT OF MUSCLE DISEASES

(71) Applicant: ANAGENESIS BIOTECHNOLOGIES S.A.S., ILLKIRCH-GRAFFENSTADEN (FR)

(72) Inventors: AURORE HICK, ILLKIRCH-GRAFFENSTADEN (FR); BÉNÉDICTE GOBERT, GEUDERTHEIM (FR); ERIC RIGUET, BRUGUIERES (FR)

(21) Appl. No.: 17/627,822

(22) PCT Filed: Jul. 17, 2020
(86) PCT No.: PCT/EP2020/070246

§ 371 Date: Jan. 18, 2022

(30) Foreign Application Priority Data
Jul. 19, 2019 (EP) 19305957.3

(51) Int. Cl. A61K 31/416; A61K 31/437; A61K 31/4439; A61K 31/444; A61K 31/4985; C07D 231/40; C07D 231/56; C07D 401/14; C07D 417/12; C07D 487/04; A61P 21/00

(52) U.S. Cl. CPC A61K 31/416; A61K 31/437; A61K 31/4439; A61K 31/444; A61K 31/4985; C07D 231/40; C07D 231/56; C07D 401/14; C07D 417/12; C07D 487/04; A61P 21/00

(57) ABSTRACT

The current invention provides urea derivatives, in particular compounds having the core structure heteroaryl-NH—CO—NH-aryl-O-heteroaryl, for use in treating, ameliorating, delaying, curing and/or preventing a disease or condition associated with muscle cells and/or satellite cells, such as Duchenne muscular dystrophy, Becker muscular dystrophy, cachexia or sarcopenia.

### FIELD

The invention relates to the field of compounds for the treatment of diseases or conditions associated with muscle cells and/or satellite cells. The invention in particular relates to the use of compounds with improved characteristics enhancing clinical applicability as further defined herein.

### BACKGROUND OF THE INVENTION

Diseases and conditions associated with muscle cells have a wide range of underlying causes and symptoms. The most common examples are inflammatory myopathies, muscular dystrophies, metabolic myopathies, myopathies associated with systemic disorders, drug-induced myopathies, cachexia and sarcopenia. In these diseases the muscle cells have a reduced biological functionality relative to muscle cells of healthy individuals, or have been degenerated.

As an example, Duchenne muscular dystrophy is a severe type of muscular dystrophy caused by a mutation in the dystrophin gene, resulting in muscle cells with a reduced biological functionality, leading to progressive muscle weakness and degeneration. As another example, sarcopenia is the loss of skeletal muscle mass due to aging.

Although diseases and conditions associated with muscle cells often have different underlying causes and biological mechanisms, a reduced number of functional satellite cells, relative to a healthy individual, have been associated with several of these diseases and conditions. Satellite cells are small multipotent cells, present in muscle tissue, characterized by their location under the basal lamina and by the expression of paired box 7 (Pax7) protein, which are precursors of skeletal muscle cells. Although these cells are quiescent under normal physiological conditions, they are activated in response to trauma and therefore play an important role in muscle repair and regeneration.

For a lot of diseases and conditions associated with muscle cells, including Duchenne muscular dystrophy and sarcopenia, there are no approved medicaments, and treatment is mostly supportive. Hence, there is a continuing need in the art for compounds which may be used in treating, ameliorating, delaying, curing and/or preventing a disease or condition associated with muscle cells and/or satellite cells.

Patent Publications WO2002032872, WO2003072569, WO2006043090, WO2006071940, WO2006081034, WO2006105844, WO2007059202, WO2007064872, WO2008046003, WO2008079972, WO2008131276, US20080113967, WO2009077766, US20090012091, WO2010067130, WO2010067131, WO2010112936, WO2011158042, WO2011070369, WO2011092469, WO2011121366, WO2011124923, WO2011124930, WO2011158039, WO2011158042, WO2011158044, WO2012019015, US20120046290, US20120129893, US20120225057, WO2013036232, WO2013050757, WO2014015056, WO2015075483, US20160015697, WO2018137610, WO2018215668, WO2019084499, WO2019232275 describe polyaromatic urea derivatives primarily as antitumor agents, anti-inflammatory agents, antiviral agents, respiratory system agents and for diabetes mellitus.

None of the specifically disclosed compounds from the above Patent Publications are included in the present invention.

### SUMMARY

The present invention provides new compounds promoting muscle progenitor differentiation, in particular improved promoting capacities, but with the ability to not deplete the pool of satellite cells, e.g., to preserve or even increase it.

The present invention relates to these compounds, a pharmaceutical composition comprising such a compound, and their uses as a drug, in particular for treating, ameliorating, delaying, curing and/or preventing a disease or condition associated with muscle cells and/or satellite cells. The present invention further relates to the use of a compound according to the present invention for the manufacture of a medicament for treating, ameliorating, delaying, curing and/or preventing a disease or condition associated with muscle cells and/or satellite cells. In addition, it relates to a method of treating, ameliorating, delaying, curing and/or preventing a disease or condition associated with muscle cells and/or satellite cells in a subject in need thereof, comprising administering a therapeutic amount of a compound according to the present invention. More particularly, the disease or condition is selected from the group consisting of Duchenne muscular dystrophy, Becker muscular dystrophy, sarcopenia and cachexia, preferably selected from the group consisting of Duchenne muscular dystrophy, Becker muscular dystrophy, and sarcopenia. In a particular aspect, the disease or condition is a muscular dystrophy such as Duchenne muscular dystrophy or Becker muscular dystrophy.

The present invention relates to a compound and its use in treating, ameliorating, delaying, curing and/or preventing a disease or condition associated with muscle cells and/or satellite cells, or a pharmaceutically acceptable salt thereof, wherein said compound is represented by structure (I),

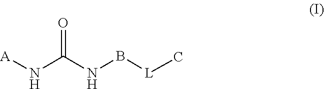

wherein

L is —O—

and

1) A is a ring system selected from the group consisting of

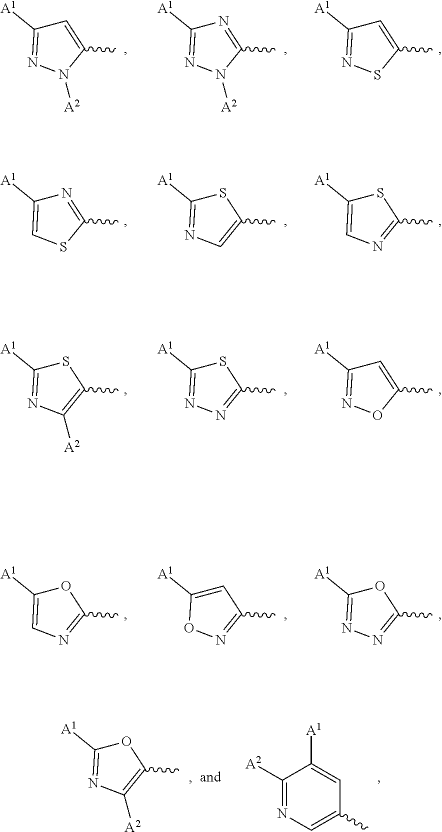

wherein A¹ is selected from the group consisting of C₁-C₄ alkyl, C₁-C₄ cycloalkyl,

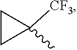

C₃-C₇ heterocycloalkyl,

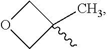

phenyl, benzyl and

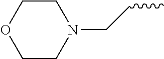

wherein A¹ is optionally fluorinated, and A² is selected from the group consisting of —H, C₁-C₄ alkyl, C₃-C₆ cycloalkyl, C₃-C₇ heterocycloalkyl, halogen, benzyl, phenyl, tolyl,

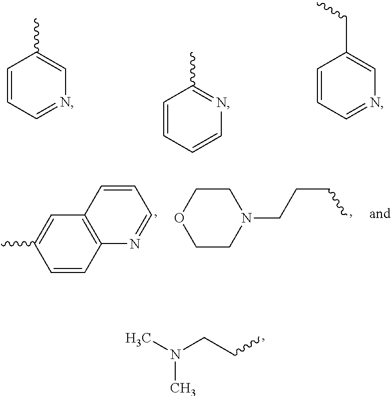

wherein A¹ and A² are optionally fluorinated, and A² is optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,

B is a ring system selected from the group consisting of

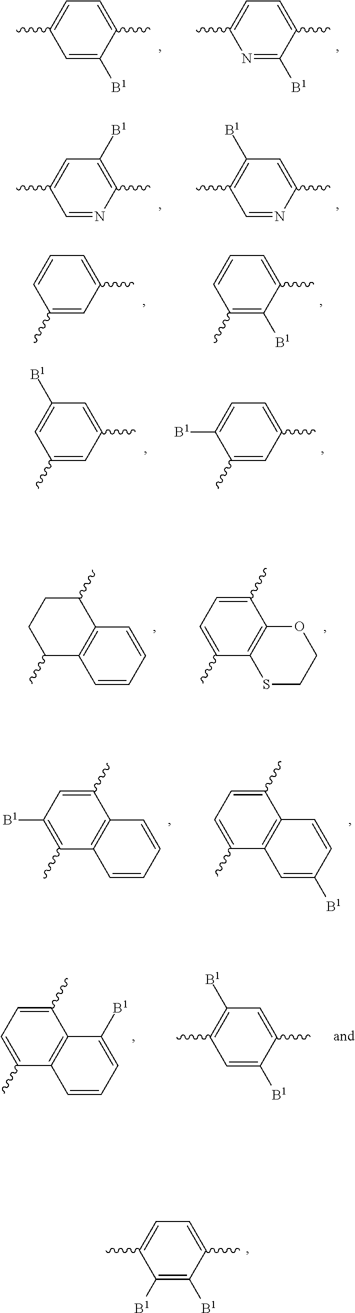

wherein B¹ is selected from the group consisting of —H, —F, —Cl, —SCH₃, —SCH₂CH₃, —CH₃, isopropyl, —CF₃, —OCH₃, —OCF₃ and wherein two B¹ can be linked to form a fused bicyclic ring system containing 0 to 3 heteroatoms; and wherein B¹ is not H when B is a phenyl;

C is selected from the group consisting of

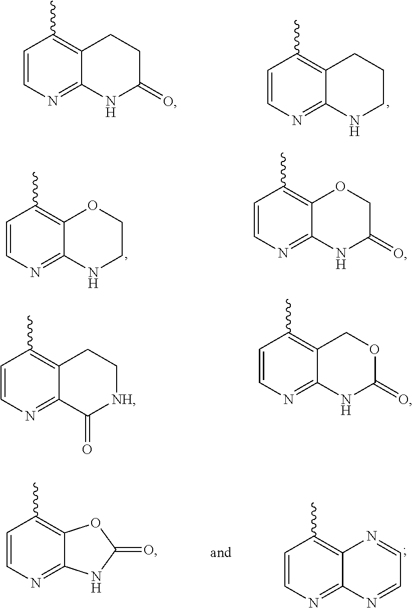

or

2) A is a ring system selected from the group consisting of

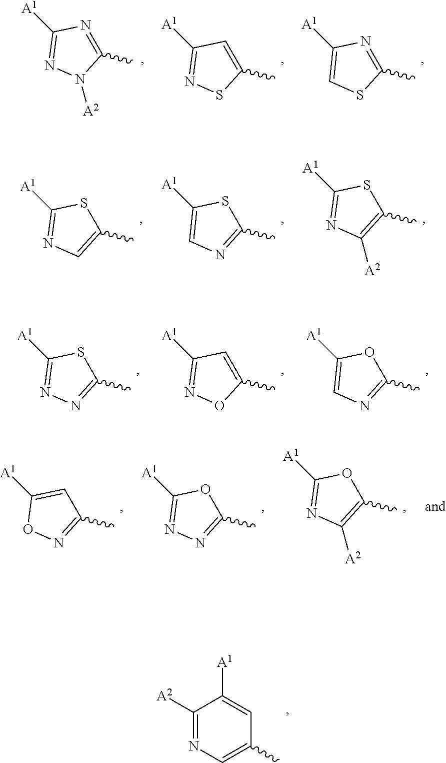

wherein A¹ is selected from the group consisting of C₁-C₄ alkyl, C₁-C₄ cycloalkyl,

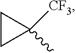

C₃-C₇ heterocycloalkyl,

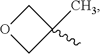

phenyl, benzyl and

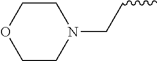

wherein A¹ is optionally fluorinated, and A² is selected from the group consisting of —H, C₁-C₄ alkyl, C₃-C₆ cycloalkyl, C₃-C₇ heterocycloalkyl, halogen, benzyl, phenyl, tolyl,

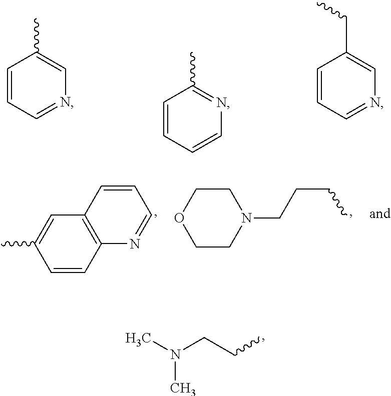

wherein A¹ and A² are optionally fluorinated, and A² is optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,

B is a ring system selected from the group consisting of

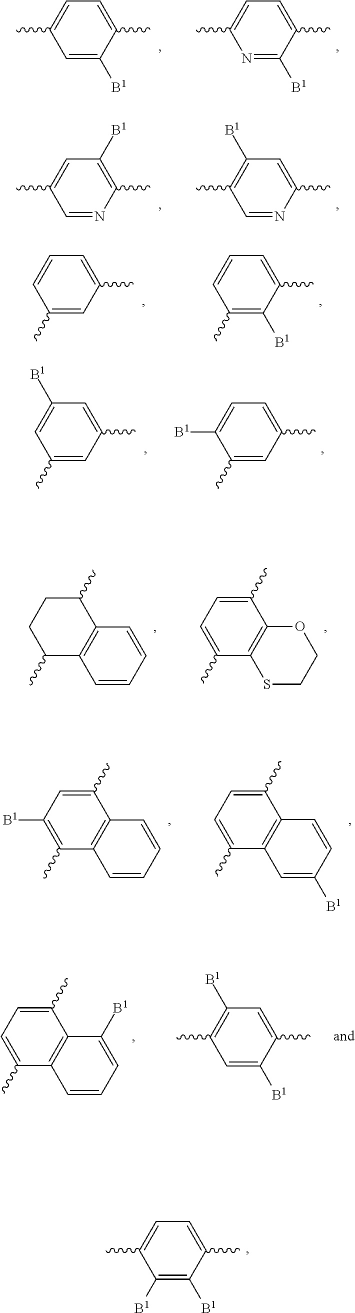

wherein B¹ is selected from the group consisting of —H, —F, —Cl, —SCH₃, —SCH₂CH₃, —CH₃, isopropyl, —CF₃, —OCH₃, —OCF₃ and wherein two B¹ can be linked to form a fused bicyclic ring system containing 0 to 3 heteroatoms; and wherein B¹ is not H when B is a phenyl;

C is selected from the group consisting of

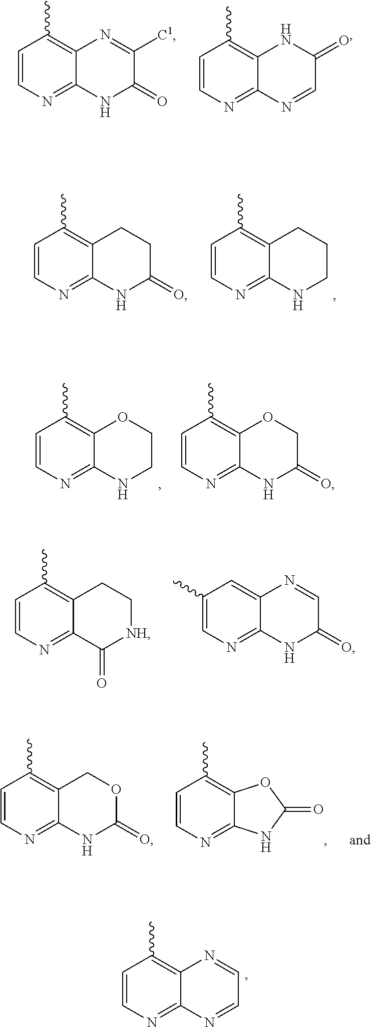

wherein C¹ is selected from the group consisting of —H, —CH₃, —OCH₃, —CN or

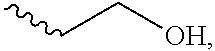

or C is selected from the group consisting of

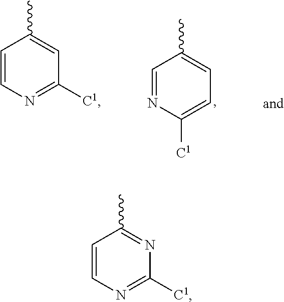

wherein C¹ is selected from the group consisting of wherein C¹ is selected from the group consisting of —H, —NH₂, —NR¹, —C(O)—NH—R¹, —NH—C(O)—R¹, —NH—S(O)₂—R¹, —NH—C(O)—OR¹, —C(O)—R¹, —R¹, —OR¹, —SO₂R¹, with R¹ being selected from the group consisting of C₁-C₁₀ alkyl, C₂-C₁₀ alkenyl, C₃-C₁₀ cycloalkyl, C₄-C₂₀ alkcycloalkyl, C₆-C₁₄ aryl, C₃-C₁₃ heteroaryl, C₇-C₂₄ alkaryl, C₃-C₇ heterocycloalkyl and C₄-C₁₇ alkheterocycloalkyl, wherein each hydrogen in each of these groups may be independently replaced by a group selected from halogen, hydroxy, —OR′, —COR′, —COOR′, and —NR′R″, each hydrogen in each of R′ and R″ may be independently replaced by —COR′″ or COOR′″, wherein R′, R″ and R′″ are independently a C₁-C₆ alkyl;

or

3) A is a ring system selected from the group consisting of

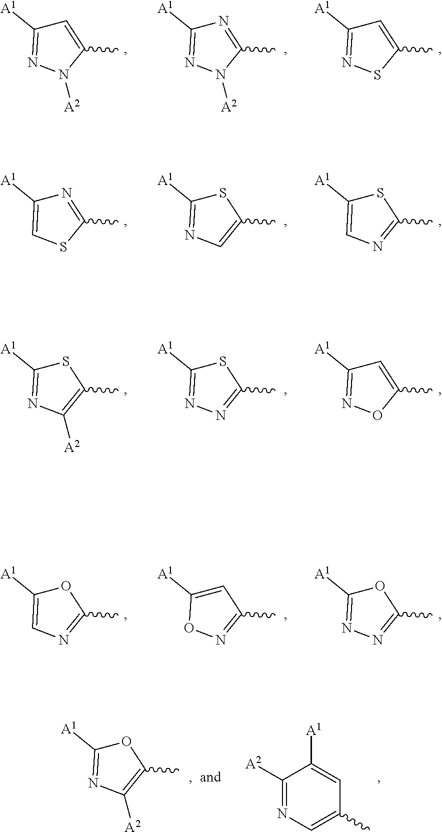

wherein A¹ is selected from the group consisting of C₁-C₄ alkyl, C₁-C₄ cycloalkyl,

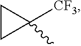

C₃-C₇ heterocycloalkyl,

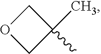

phenyl, benzyl and

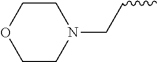

wherein A¹ is optionally fluorinated, and A² is selected from the group consisting of —H, C₁-C₄ alkyl, C₃-C₆ cycloalkyl, C₃-C₇ heterocycloalkyl, halogen, benzyl, phenyl, tolyl,

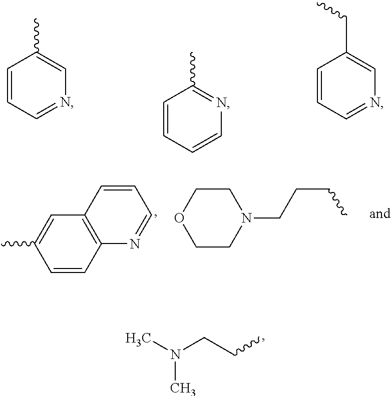

wherein A¹ and A² are optionally fluorinated, and A² is optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,

B is a ring system selected from the group consisting of

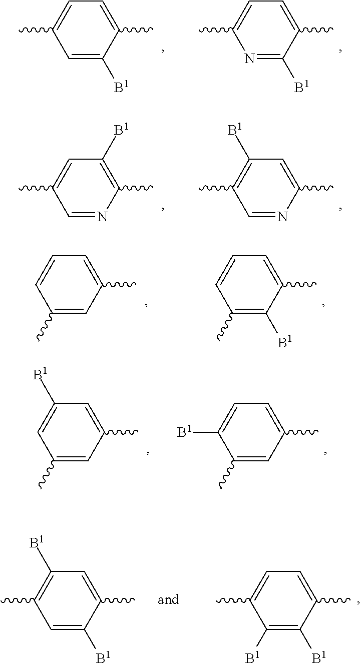

wherein B¹ is selected from the group consisting of —CF₃, —OCF₃, —OCH₃, and SCH₂CH₃, or B has two B¹ groups selected independently from the group consisting of —F, —Cl, —SCH₃, —SCH₂CH₃, —CH₃, isopropyl, —CF₃, —OCH₃, —OCF₃,

C is selected from the group consisting of

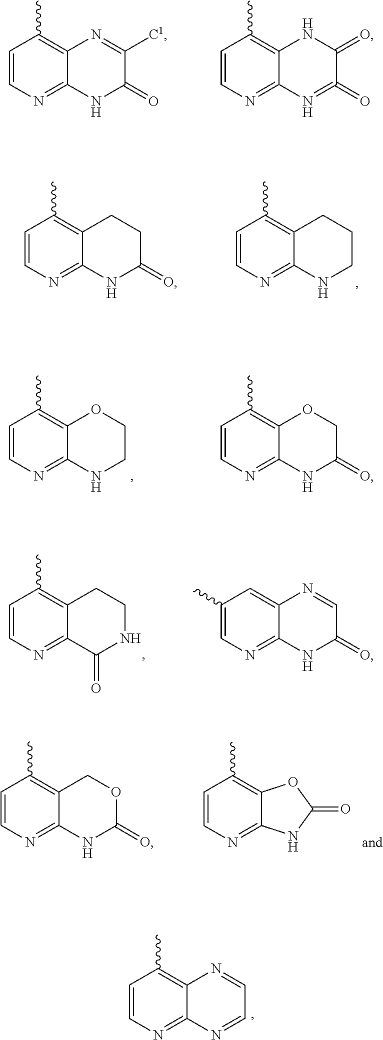

wherein C¹ is selected from the group consisting of —H, —CH₃, —OCH₃, —CN or

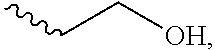

or C is selected from the group consisting of

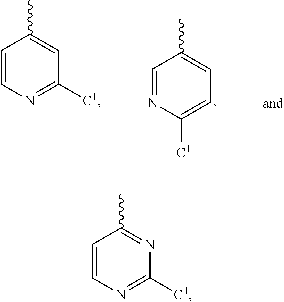

wherein C¹ is selected from the group consisting of wherein C¹ is selected from the group consisting of —H, —NH₂, —NR¹, —C(O)—NH—R¹, —NH—C(O)—R¹, —NH—S(O)₂—R¹, —NH—C(O)—OR¹, —C(O)—R¹, —R¹, —OR¹, —SO₂R¹, with R¹ being selected from the group consisting of C₁-C₁₀ alkyl, C₂-C₁₀ alkenyl, C₃-C₁₀ cycloalkyl, C₄-C₂₀ alkcycloalkyl, C₆-C₁₄ aryl, C₃-C₁₃ heteroaryl, C₇-C₂₄ alkaryl, C₃-C₇ heterocycloalkyl and C₄-C₁₇ alkheterocycloalkyl, wherein each hydrogen in each of these groups may be independently replaced by a group selected from halogen, hydroxy, —OR′, —COR′, —COOR′, and —NR′R″, each hydrogen in each of R′ and R″ may be independently replaced by —COR′″ or COOR′″, wherein R′, R″ and R′″ are independently a C₁-C₆ alkyl;

or

4) A is

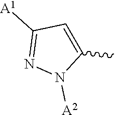

wherein A¹ is selected from the group consisting of C₁-C₄ alkyl, C₁-C₄ cycloalkyl,

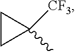

C₃-C₇ heterocycloalkyl,

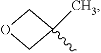

phenyl, benzyl and

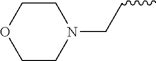

wherein A¹ is optionally fluorinated, and A² is selected from the group consisting of a phenyl substituted with 1, 2 or 3 substituents selected from the group consisting of —Cl, —F, —CN, C₁-C₃ alkyloxy optionally substituted by C₃-C₇ heterocycloalkyl or NRR, with R being a C₁-C₃ alkyl, and C₁-C₄ alkyl substituted by C₃-C₇ heterocycloalkyl or NRR, with R being a C₁-C₃ alkyl; a phenyl or pyridinyl substituted by 2 or 3 C₁-C₃ alkyl; and a pyridinyl substituted with 1, 2 or 3 substituents selected from the group consisting of —Cl, —F, —CN, C₁-C₁₀ alkyloxy substituted by C₃-C₇ heterocycloalkyl or NRR, with R being a C₁-C₃ alkyl, and C₁-C₄ alkyl substituted by C₃-C₇ heterocycloalkyl or NRR, with R being a C₁-C₃ alkyl;

B is a ring system selected from the group consisting of

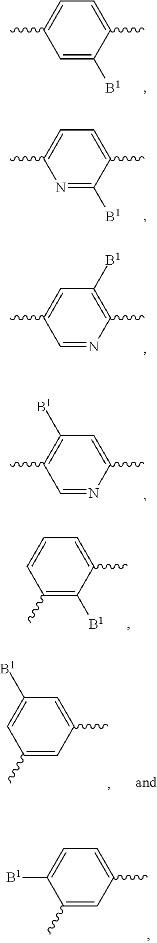

wherein B¹ is —SCH₃,

C is selected from the group consisting of

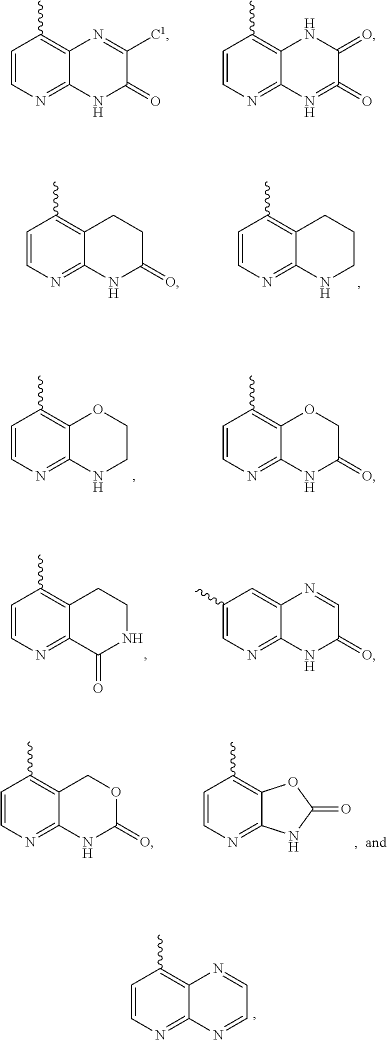

wherein C¹ is selected from the group consisting of —H, —CH₃, —OCH₃, —CN or

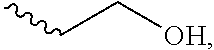

or

5) A is

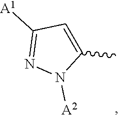

and

A¹ is selected from the group consisting of C₁-C₄ alkyl, C₃-C₆ cycloalkyl,

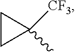

C₃-C₇ heterocycloalkyl,

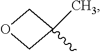

phenyl, benzyl and

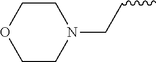

wherein A¹ is optionally fluorinated,

A² is

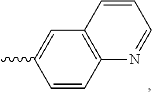

B is a ring system selected from the group consisting of

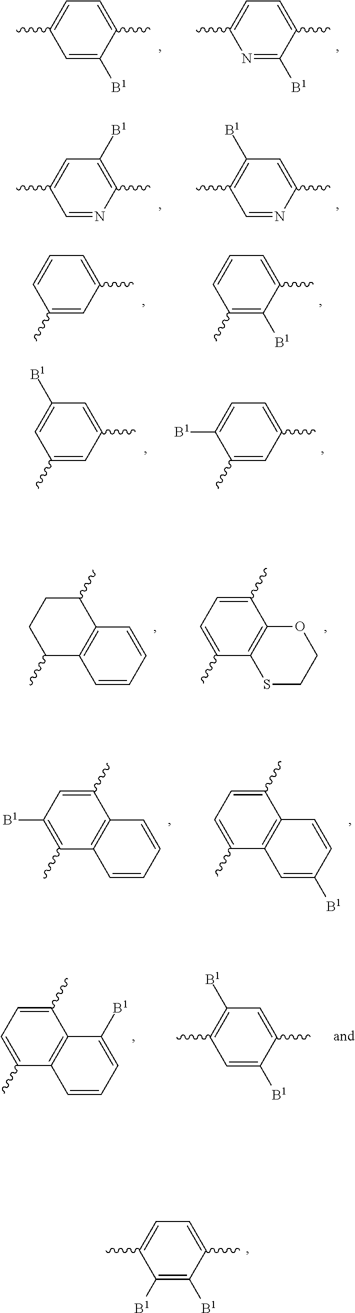

wherein B¹ is selected from the group consisting of —H, —F, —Cl, —SCH₃, —SCH₂CH₃, —CH₃, isopropyl, —CF₃, —OCH₃, —OCF₃ and wherein two B¹ can be linked to form a fused bicyclic ring system containing 0 to 3 heteroatoms; and wherein B¹ is not H when B is a phenyl;

C is selected from the group consisting of

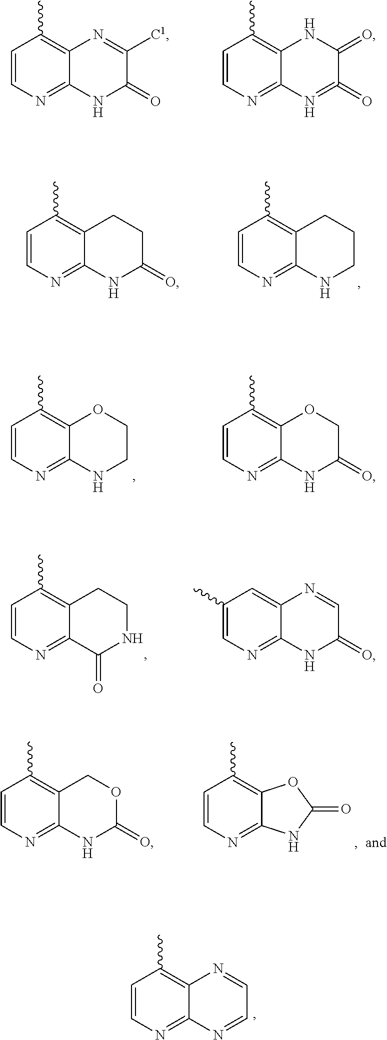

wherein C¹ is selected from the group consisting of —H, —CH₃, —OCH₃, —CN or

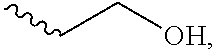

or

6) A is a ring system selected from the group consisting of

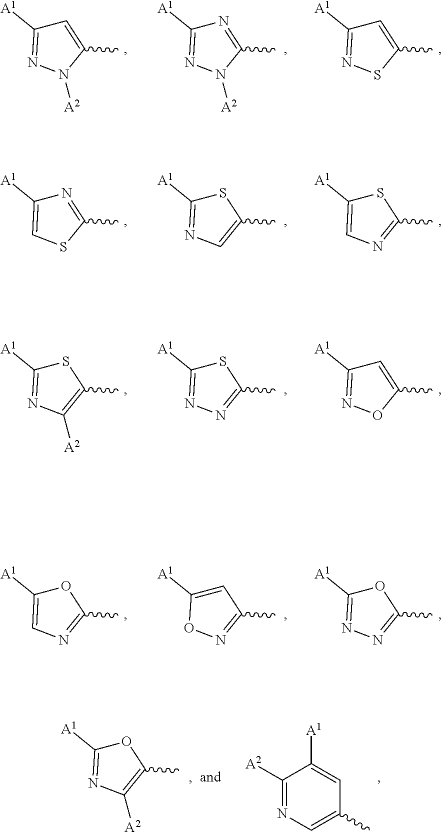

wherein A¹ is selected from the group consisting of C₁-C₄ alkyl, C₁-C₄ cycloalkyl,

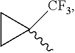

C₃-C₇ heterocycloalkyl,

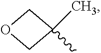

phenyl, benzyl and

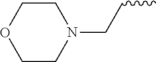

wherein A¹ is optionally fluorinated, and A² is selected from the group consisting of —H, C₁-C₄ alkyl, C₃-C₆ cycloalkyl, C₃-C₇ heterocycloalkyl, halogen, benzyl, phenyl, tolyl,

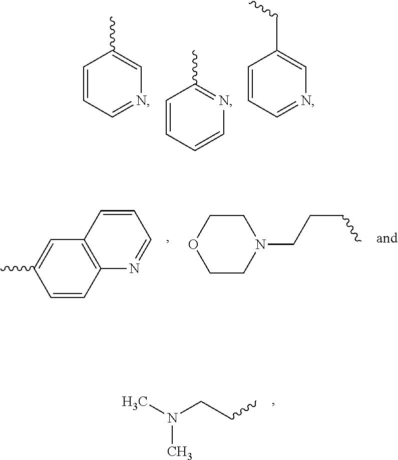

wherein A¹ and A² are optionally fluorinated, and A² is optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,

B is a ring system selected from the group consisting of

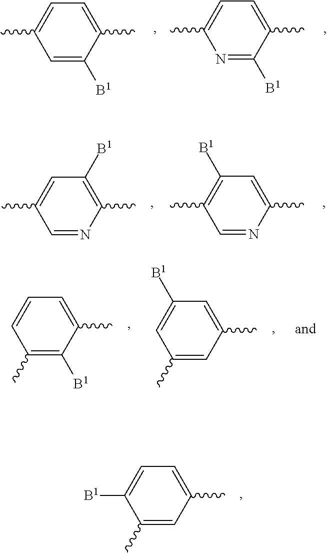

wherein B¹ is —SCH₃,

C is selected from the group consisting of

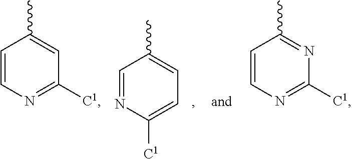

wherein C¹ is selected from the group consisting of wherein C¹ is selected from the group consisting of —H, —NH₂, —NR¹, —C(O)—NH—R¹, —NH—C(O)—R¹, —NH—S(O)₂—R¹, —NH—C(O)—OR¹, —C(O)—R¹, —R¹, —OR¹, —SO₂R¹, with R¹ being selected from the group consisting of C₁-C₁₀ alkyl, C₂-C₁₀ alkenyl, C₃-C₁₀ cycloalkyl, C₄-C₂₀ alkcycloalkyl, C₆-C₁₄ aryl, C₃-C₁₃ heteroaryl, C₇-C₂₄ alkaryl, C₃-C₇ heterocycloalkyl and C₄-C₁₇ alkheterocycloalkyl, wherein each hydrogen in each of these groups may be independently replaced by a group selected from halogen, hydroxy, —OR′, —COR′, —COOR′, and —NR′R″, each hydrogen in each of R′ and R″ may be independently replaced by —COR′″ or COOR′″, wherein R′, R″ and R′″ are independently a C₁-C₆ alkyl;

or

7) A is a ring system selected from the group consisting of

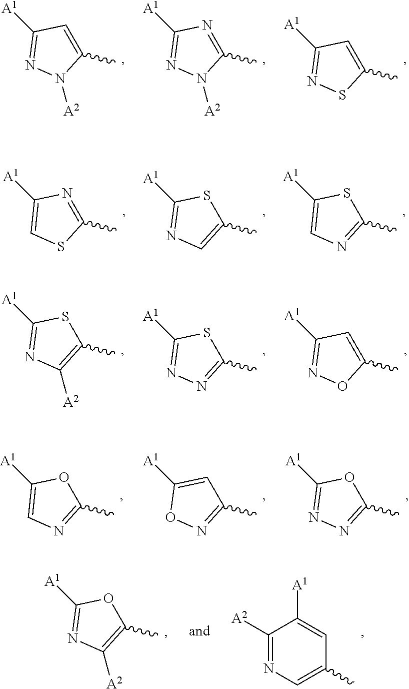

wherein A¹ is selected from the group consisting of C₁-C₄ alkyl, C₁-C₄ cycloalkyl,

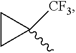

C₃-C₇ heterocycloalkyl,

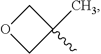

phenyl, benzyl and

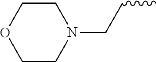

wherein A¹ is optionally fluorinated, and A² is selected from the group consisting of —H, C₁-C₄ alkyl, C₃-C₆ cycloalkyl, C₃-C₇ heterocycloalkyl, halogen, benzyl, phenyl, tolyl,

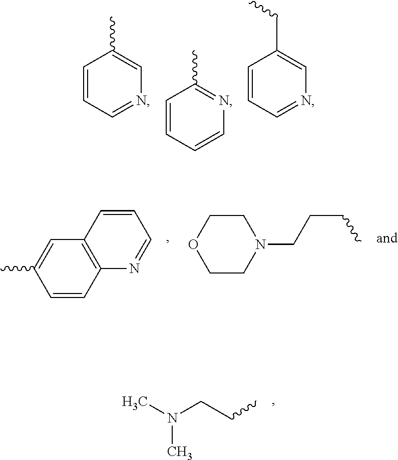

wherein A¹ and A² are optionally fluorinated, and A² is optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,

B is a ring system selected from the group consisting of

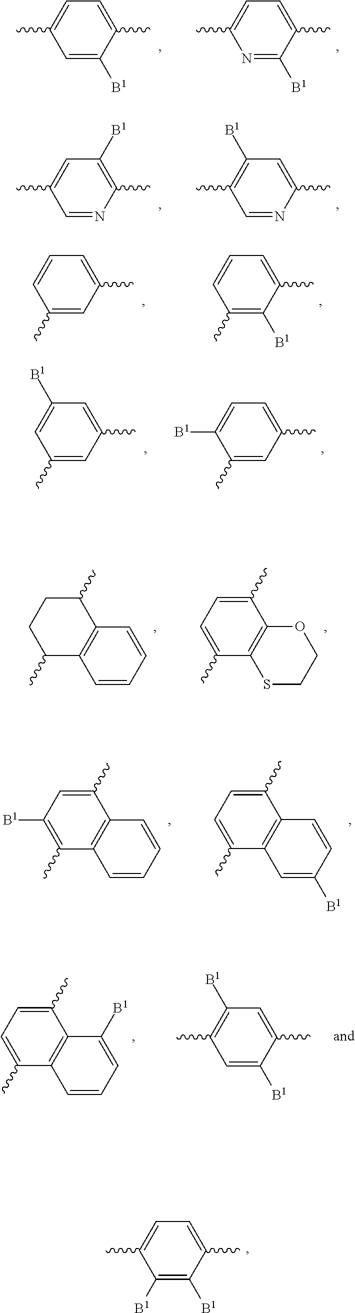

wherein B¹ is selected from the group consisting of —H, —F, —Cl, —SCH₃, —SCH₂CH₃, —CH₃, isopropyl, —CF₃, —OCH₃, —OCF₃ and wherein two B¹ can be linked to form a fused bicyclic ring system containing 0 to 3 heteroatoms; and wherein B¹ is not H when B is a phenyl;

C is selected from the group consisting of

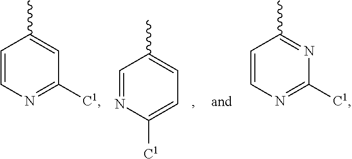

wherein C¹ is selected from the group consisting of —NH—C(O)R¹ or —NH—C(O)—OR¹ with R¹ being selected from the group consisting of C₁-C₁₀ alkyl, C₆-C₁₄ aryl, C₃-C₁₃ heteroaryl, and C₇-C₂₄ alkaryl, wherein each hydrogen in each of these groups may be independently replaced by a group selected from halogen, hydroxy, —OR′, and —NR′R″, each hydrogen in each of R′ and R″ may be independently replaced by —COR′″ or COOR′″, wherein R′, R″ and R′″ are independently a C₁-C₆ alkyl.

In a first aspect, the compound has a structure of formula (I) in which

L is —O—

A is a ring system selected from the group consisting of

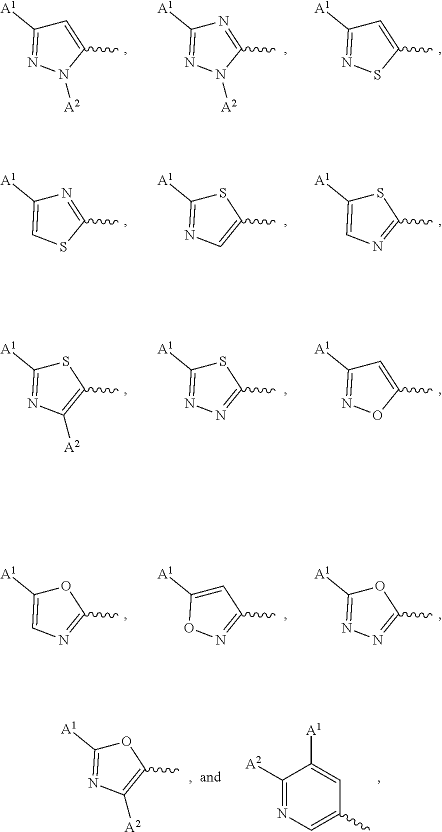

A¹ is selected from the group consisting of methyl, —CF₃, isopropyl, cyclopropyl, isobutyl, sec-butyl, tert-butyl,

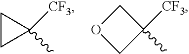

and A² is selected from the group consisting of H, methyl, iso-propyl, benzyl, phenyl, chlorine,

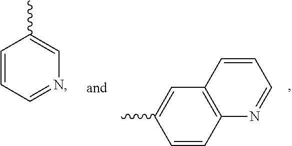

wherein A¹ and A² are optionally fluorinated, and A₂ is optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,

B is a ring system selected from the group consisting of

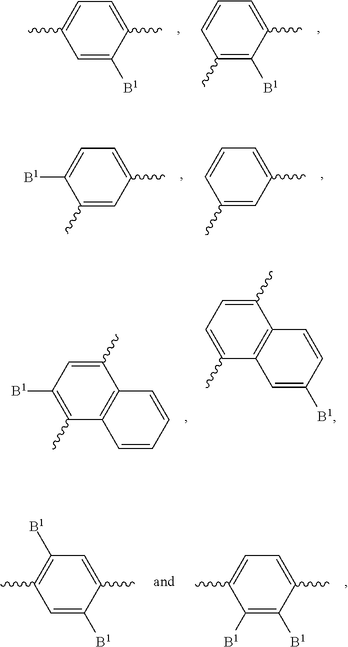

wherein, when B is a phenyl, B¹ is selected from the group consisting of —F, —Cl, —SCH₃, —SCH₂CH₃, —CH₃, —CF₃, —OCH₃, and —OCF₃ and, when B is a naphtyl, B¹ is —H, and

C is selected from the group consisting of

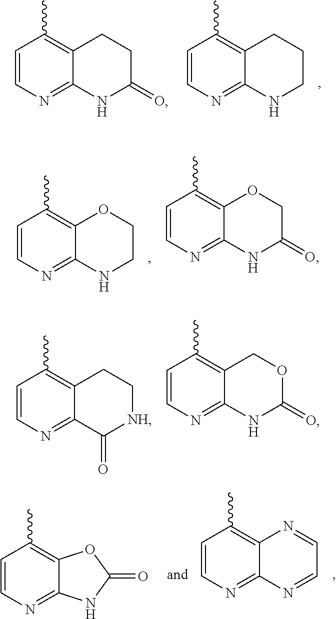

In a particular first aspect, the compound has a structure of formula (I) in which L is —O—

A is a ring selected from the group consisting of

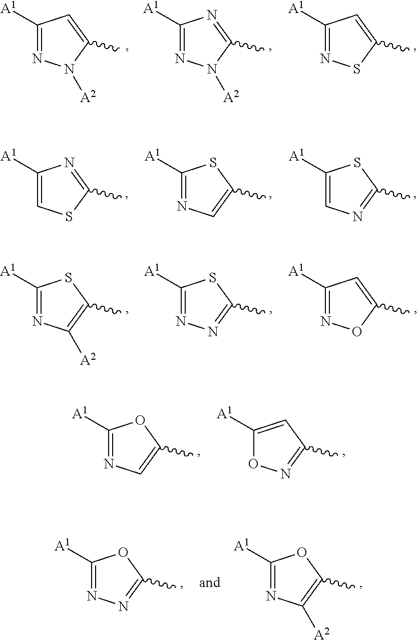

A¹ is tert-butyl,

A² is a phenyl or

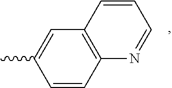

optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,

B is a ring system selected from the group consisting of

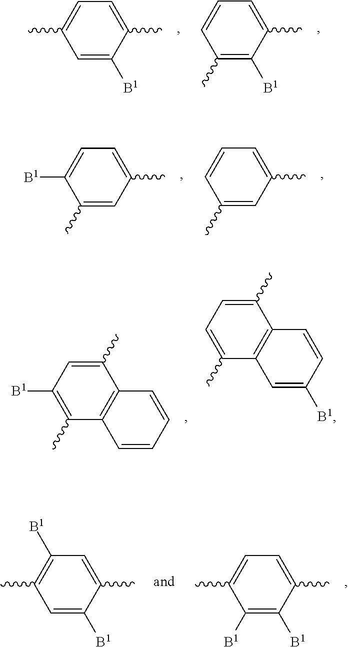

wherein, when B is a phenyl, B¹ is selected from the group consisting of —F, —SCH₃, —SCH₂CH₃, —CF₃, —OCH₃, and —OCF₃ and, when B is a naphtyl, B¹ is —H, and

C is selected from the group consisting of

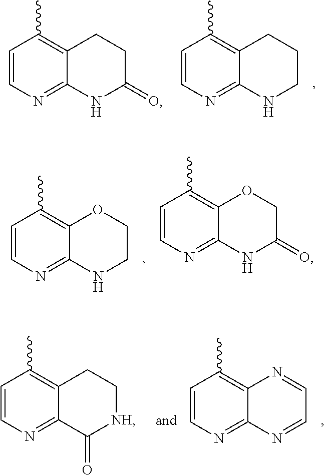

In a particular first aspect, the compound has a structure of formula (I) in which

L is —O—

A is a ring selected from the group consisting of

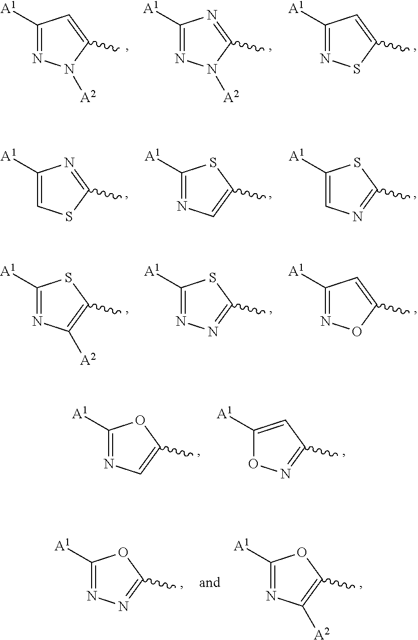

A¹ is tert-butyl,

A² is a phenyl or

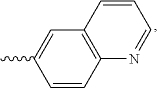

optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,

B is a ring system selected from the group consisting of

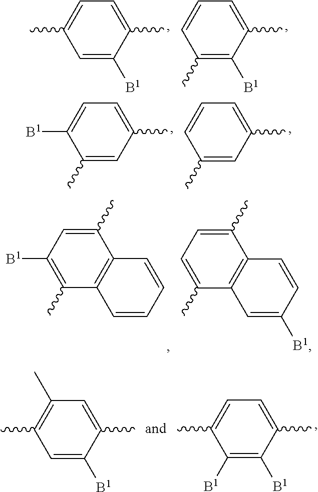

wherein, when B is a phenyl, B¹ is selected from the group consisting of —F, —SCH₃, —SCH₂CH₃, —CF₃, —OCH₃, and —OCF₃ and, when B is a naphtyl, B¹ is —H, and

C is selected from the group consisting of

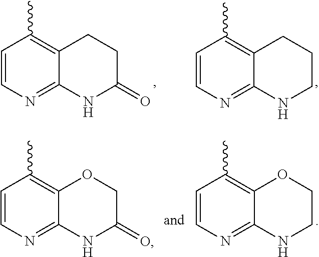

In a particular first aspect, the compound has a structure of formula (I) in which

L is —O—

A is a ring selected from the group consisting of

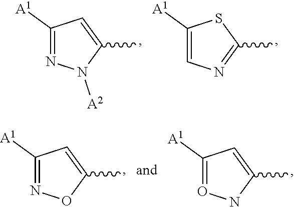

A¹ is tert-butyl,

A² is a phenyl optionally substituted with 1, 2 or 3 substituents selected from the group consisting of —CH₃, —Cl, —F, —CN, —OCH₃, —O—(CH₂)₂-piperidinyl, —O—(CH₂)₂-morpholinyl, —O—(CH₂)₃—N(CH₃)₂, and —CH₂-morpholinyl, or A² is

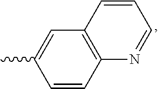

B is a ring system selected in the group consisting of

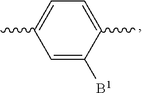

with B¹ being selected from the group consisting of —F, —SCH₃, —SCH₂CH₃, —CF₃, —OCH₃, and —OCF₃, preferably —SCH₃,

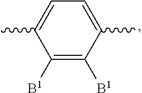

with B¹ being F,

and

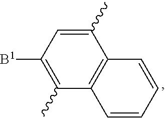

with B¹ being —H, and

C is selected from the group consisting of

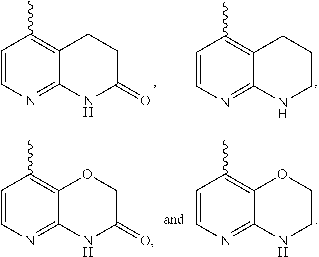

In the first aspect, the compound can be selected in the group consisting of
1-(3-(tert-butyl)-1-phenyl-1H-pyrazol-5-yl)-3-(2-(methylthio)-4-((7-oxo-5,6,7,8-tetrahydro-1,8-naphthyridin-4-yl)oxy)phenyl)urea;
1-(3-(tert-butyl)-1-phenyl-1H-pyrazol-5-yl)-3-(2-(methylthio)-4-((5,6,7,8-tetrahydro-1,8-naphthyridin-4-yl)oxy)phenyl)urea;
1-(3-(tert-butyl)-1-(p-tolyl)-1H-pyrazol-5-yl)-3-(2-(methylthio)-4-((7-oxo-5,6,7,8-tetrahydro-1,8-naphthyridin-4-yl)oxy)phenyl)urea;
1-(3-(tert-butyl)-1-(3-chlorophenyl)-1H-pyrazol-5-yl)-3-(2-(methylthio)-4-((7-oxo-5,6,7,8-tetrahydro-1,8-naphthyridin-4-yl)oxy)phenyl)urea
1-(5-(tert-butyl)thiazol-2-yl)-3-(2-(methylthio)-4-((7-oxo-5,6,7,8-tetrahydro-1,8-naphthyridin-4-yl)oxy)phenyl)urea
1-(3-(tert-butyl)-1-phenyl-1H-pyrazol-5-yl)-3-(2-(methylthio)-4-((3-oxo-3,4-dihydro-2H-pyrido[3,2-b][1,4]oxazin-8-yl)oxy)phenyl)urea;
1-(5-(tert-butyl)thiazol-2-yl)-3-(4-((7-oxo-5,6,7,8-tetrahydro-1,8-naphthyridin-4-yl)oxy)naphthalen-1-yl)urea;
1-(3-(tert-butyl)-1-phenyl-1H-pyrazol-5-yl)-3-(4-((3,4-dihydro-2H-pyrido[3,2-b][1,4]oxazin-8-yl)oxy)-2-(methylthio)phenyl)urea;
1-(3-(tert-butyl)isoxazol-5-yl)-3-(2-(methylthio)-4-((7-oxo-5,6,7,8-tetrahydro-1,8-naphthyridin-4-yl)oxy)phenyl)urea;
1-(3-(tert-butyl)isoxazol-5-yl)-3-(4-((7-oxo-5,6,7,8-tetrahydro-1,8-naphthyridin-4-yl)oxy)naphthalen-1-yl)urea; and
1-(3-(tert-butyl)-1-phenyl-1H-pyrazol-5-yl)-3-(2-(methylthio)-4-((7-oxo-7,8-dihydro-1,8-naphthyridin-4-yl)oxy)phenyl)urea

or a pharmaceutically acceptable salt thereof.

In a second aspect, the compound has a structure of formula (I) in which

L is —O—

A is a ring selected from the group consisting of

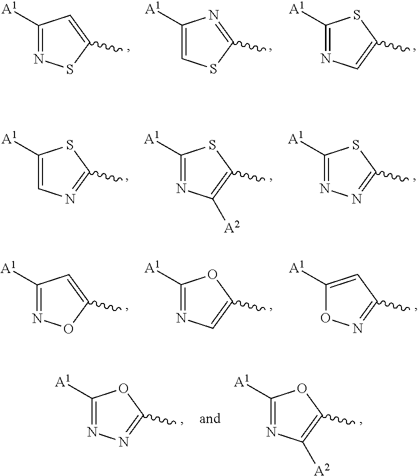

A¹ is selected from the group consisting of methyl, —CF₃, isopropyl, cyclopropyl, isobutyl, sec-butyl, tert-butyl,

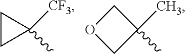

A² is —H,

B is a ring system selected from the group consisting of

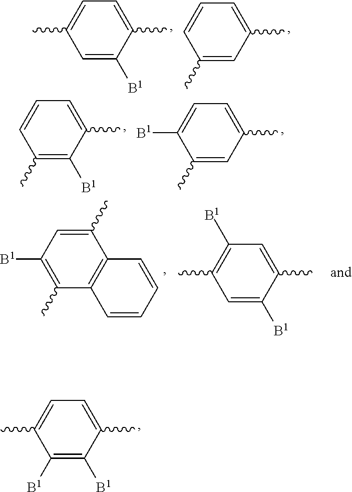

wherein, when B is a phenyl, B¹ is selected from the group consisting of —F, —Cl, —SCH₃, —SCH₂CH₃, —CH₃, —CF₃, —OCH₃, and —OCF₃ and, when B is a naphtyl, B¹ is —H, and

C is selected from the group consisting of

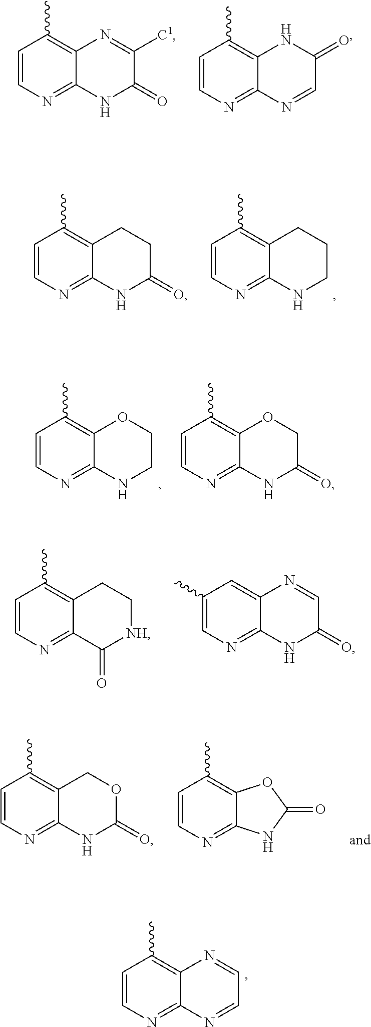

wherein C¹ is selected from the group consisting of —H, —CH₃, —OCH₃, —CN or

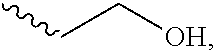

or

C is selected from the group consisting of

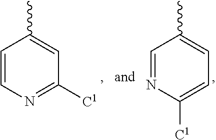

with C¹ being selected C¹ is selected from the group consisting of wherein C¹ is selected from the group consisting of —H, —NH₂, —NR¹, —C(O)—NH—R¹, —NH—C(O)—R¹, —NH—S(O)₂—R¹, —NH—C(O)—OR¹, —C(O)—R¹, —R¹, —OR¹, —SO₂R¹, with R¹ being selected from the group consisting of C₁-C₁₀ alkyl, C₆-C₁₄ aryl, C₃-C₁₃ heteroaryl, and C₇-C₂₄ alkaryl, wherein each hydrogen in each of these groups may be independently replaced by a group selected from halogen, hydroxy, —OR′, and —NR′R″, each hydrogen in each of R′ and R″ may be independently replaced by —COR′″ or COOR′″, wherein R′, R″ and R′″ are independently a C₁-C₆ alkyl.

In a particular second aspect, the compound has a structure of formula (I) in which

L is —O—

A is a ring selected from the group consisting of

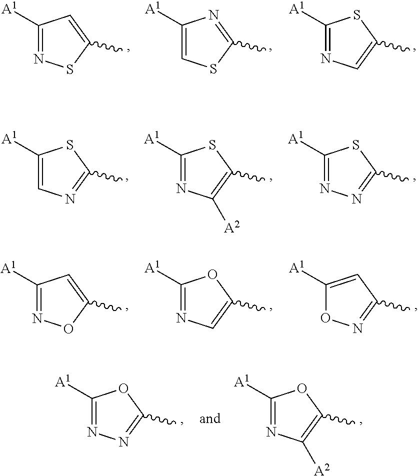

A¹ is tert-butyl,

A² is —H,

B is a ring system selected from the group consisting of

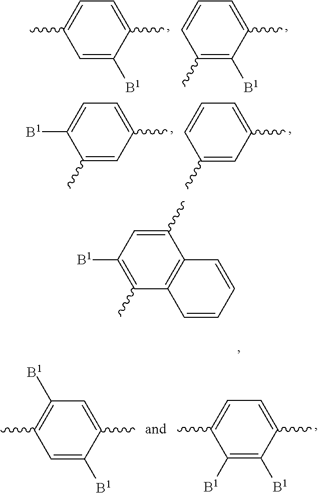

wherein, when B is a phenyl, B¹ is selected from the group consisting of —F, —SCH₃, —SCH₂CH₃, —CF₃, —OCH₃, and —OCF₃ and, when B is a naphtyl, B¹ is —H, and

C is selected from the group consisting of

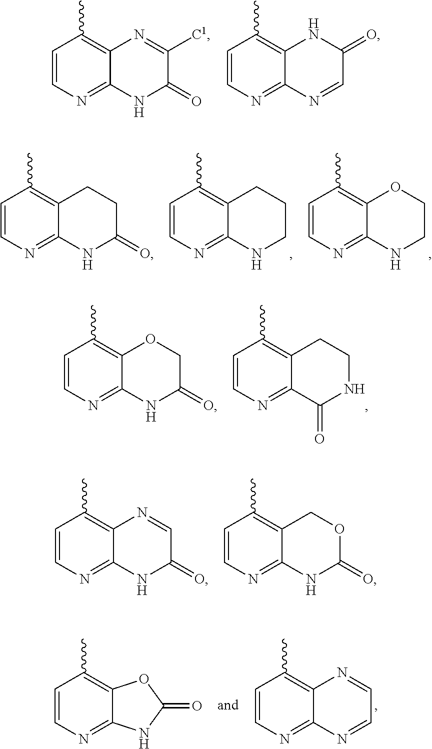

wherein C^{C} is selected from the group consisting of —H, —CH₃, —OCH₃, —CN or

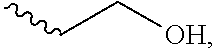

or

C is selected from the group consisting of

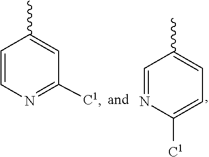

with C¹ being selected from the group consisting of —H, —NH₂,

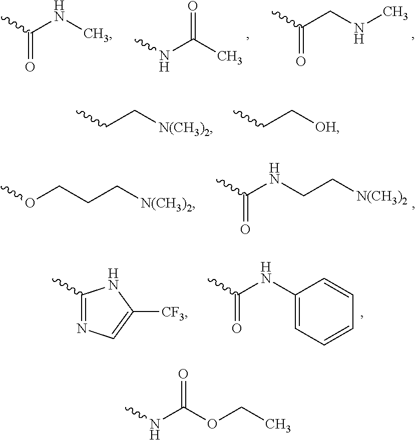

—NH—C(O)—OtBu, —NH—C(O)—N(CH₃)—C(O)—OtBu, —NH—C(O)—CH₂—N(CH₃)—C(O)—OtBu, —NH—C(O)—CH₂—NHCH₃, —NH—C(O)—Obenzyl, —NH—C(O)—CH₂—OH, —NH—(CH₂)₂—N(CH₃)₂, and —NH—S(O)₂—CH₃; preferably from the group consisting of —H,

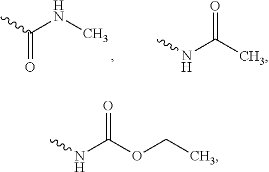

—NH—C(O)—OtBu, —NH—C(O)—N(CH₃)—C(O)—OtBu, —NH—C(O)—CH₂—N(CH₃)—C(O)—OtBu, —NH—C(O)—CH₂—NHCH₃, —NH—C(O)—Obenzyl, and —NH—C(O)—CH₂—OH.

In a particular second aspect, the compound has a structure of formula (I) in which

L is —O—

A is selected in the group consisting of

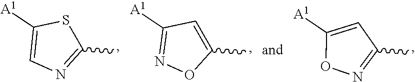

A¹ is tert-butyl,

B is a ring system selected in the group consisting of

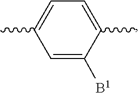

with B¹ being selected from the group consisting of —F, —SCH₃, —SCH₂CH₃, —CF₃, —OCH₃, and —OCF₃, preferably —SCH₃,

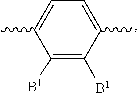

with B¹ being F,

and

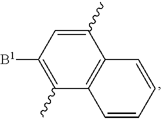

with B¹ being —H, and

C is selected in the group consisting of

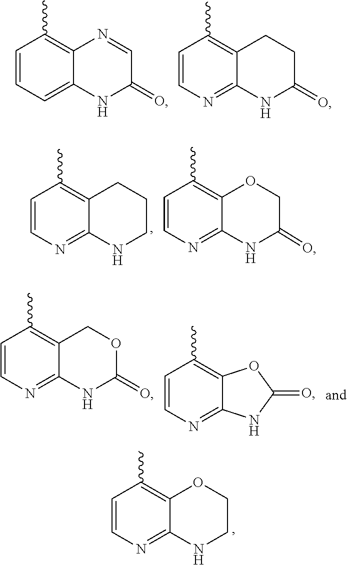

or C is selected from the group consisting of

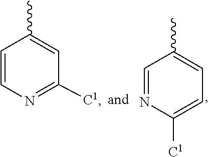

with C¹ being selected from the group consisting of

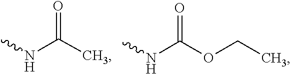

—NH—C(O)—OtBu, —NH—C(O)—N(CH₃)—COOtBu and —NH—C(O)—CH₂—N(CH₃)—C(O)—OtBu.

In a particular second aspect, the compound can be selected in the group consisting of
1-(5-(tert-butyl)thiazol-2-yl)-3-(4-((3-oxo-3,4-dihydropyrido[2,3-b]pyrazin-8-yl)oxy)naphthalen-1-yl)urea;
1-(3-(tert-butyl)isoxazol-5-yl)-3-(2-(methylthio)-4-((3-oxo-3,4-dihydropyrido[2,3-b]pyrazin-8-yl)oxy)phenyl)urea;
1-(5-(tert-butyl)thiazol-2-yl)-3-(2-(methylthio)-4-((7-oxo-5,6,7,8-tetrahydro-1,8-naphthyridin-4-yl)oxy)phenyl)urea;
1-(5-(tert-butyl)thiazol-2-yl)-3-(4-((7-oxo-5,6,7,8-tetrahydro-1,8-naphthyridin-4-yl)oxy)naphthalen-1-yl)urea;
1-(3-(tert-butyl)isoxazol-5-yl)-3-(2-(methylthio)-4-((7-oxo-5,6,7,8-tetrahydro-1,8-naphthyridin-4-yl)oxy)phenyl)urea;
ethyl (4-(4-(3-(3-(tert-butyl)isoxazol-5-yl)ureido)-3-(methylthio)phenoxy)pyridin-2-yl)carbamate;
ethyl (4-(4-(3-(5-(tert-butyl)thiazol-2-yl)ureido)-3-(methylthio)phenoxy)pyridin-2-yl)carbamate; 1-(3-(tert-butyl)isoxazol-5-yl)-3-(4-((7-oxo-5,6,7,8-tetrahydro-1,8-naphthyridin-4-yl)oxy)naphthalen-1-yl)urea; and
ethyl (4-(4-(3-(5-(tert-butyl)isoxazol-3-yl)ureido)-3-(methylthio)phenoxy)pyridin-2-yl)carbamate;

or a pharmaceutically acceptable salt thereof.

In a third aspect, the compound has a structure of formula (I) in which

L is —O—

A is a ring system selected from the group consisting of

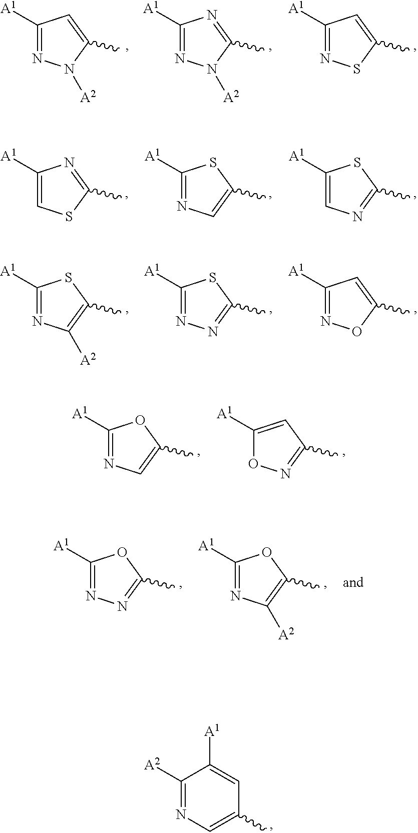

A¹ is selected from the group consisting of methyl, —CF₃, isopropyl, cyclopropyl, isobutyl, sec-butyl, tert-butyl,

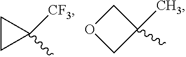

and A² is selected from the group consisting of H, methyl, iso-propyl, benzyl, phenyl, chlorine,

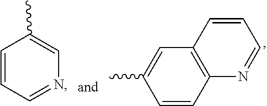

wherein A¹ and A² are optionally fluorinated, and A² is optionally substituted with 1, 2 or 3 substituents selected from the group consisting of C₁-C₄ alkyl, —Cl, —F, —CN, —OR^{A}, and —SO₂R^{A}, wherein R^{A} is C₁-C₁₀ alkyl, and the substituent being optionally substituted by C₃-C₇ heterocycloalkyl or NRR, with R being a C₁-C₃ alkyl,

B is a ring system selected from the group consisting of

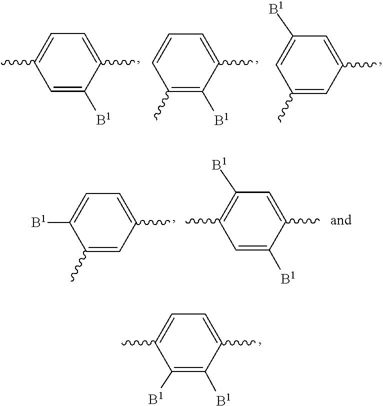

wherein B¹ is selected from the group consisting of —CF₃, —OCF₃, —OCH₃, and SCH₂CH₃, or B has two B¹ groups selected independently from the group consisting of —F, —Cl, —SCH₃, —SCH₂CH₃, —CH₃, isopropyl, —CF₃, —OCH₃, —OCF₃,

C is selected from the group consisting of

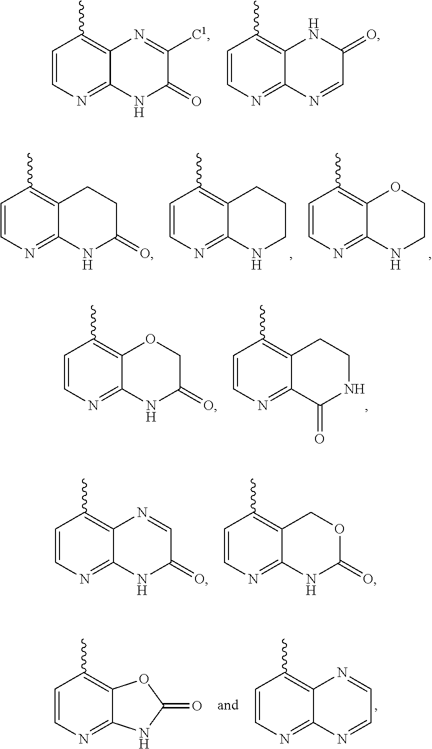

wherein C¹ is selected from the group consisting of —H, —CH₃, —OCH₃, —CN or

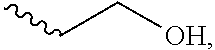

or

C is selected from the group consisting of

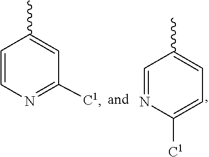

with C¹ being selected C¹ is selected from the group consisting of wherein C¹ is selected from the group consisting of —H, —NH₂, —NR¹, —C(O)—NH—R¹, —NH—C(O)—R¹, —NH—S(O)₂—R¹, —NH—C(O)—OR¹, —C(O)—R¹, —R¹, —OR¹, —SO₂R¹, with R¹ being selected from the group consisting of C₁-C₁₀ alkyl, C₆-C₁₄ aryl, C₃-C₁₃ heteroaryl, and C₇-C₂₄ alkaryl, wherein each hydrogen in each of these groups may be independently replaced by a group selected from a halogen, a hydroxy, —OR′, and —NR′R″, each hydrogen in each of R′ and R″ may be independently replaced by —COR′″ or COOR′″, wherein R′, R″ and R′″ are independently a C₁-C₆ alkyl.

In a particular third aspect, the compound has a structure of formula (I) in which

L is —O—

A is a ring selected from the group consisting of

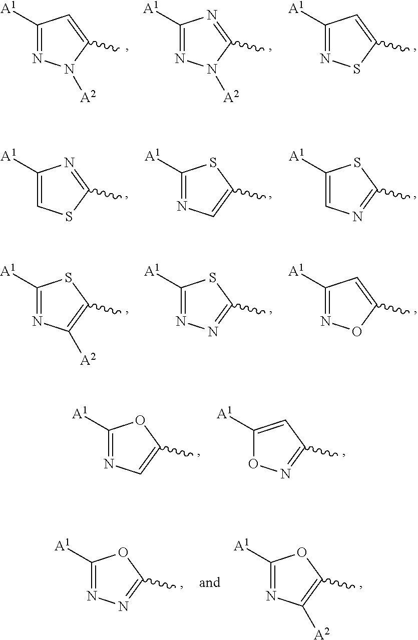

A¹ is tert-butyl,

A² is a phenyl or

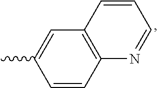

optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,

B is a ring system selected from the group consisting of

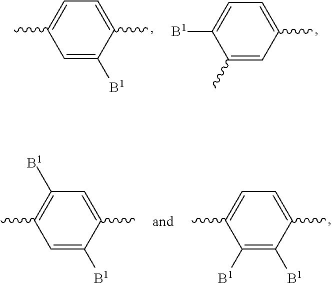

wherein B¹ is selected from the group consisting of —CF₃, —OCF₃, —OCH₃, and SCH₂CH₃, or B has two B¹ groups selected independently from the group consisting of —F, —Cl, —SCH₃, —SCH₂CH₃, —CH₃, isopropyl, —CF₃, —OCH₃, —OCF₃,

C is selected from the group consisting of

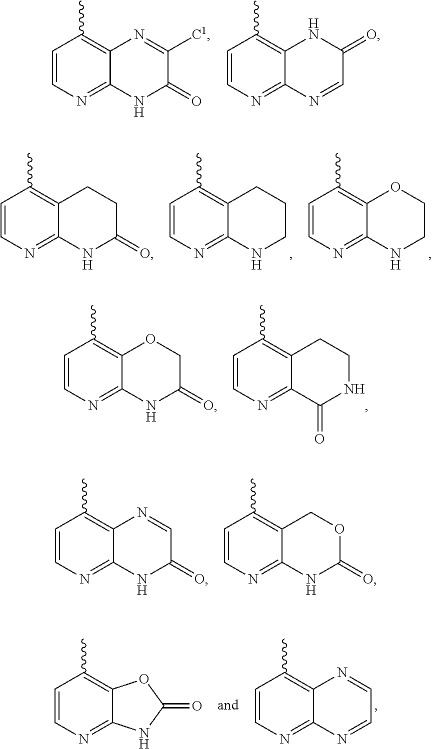

wherein C¹ is selected from the group consisting of —H, —CH₃, —OCH₃, —CN or

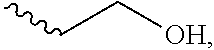

or

C is selected from the group consisting of

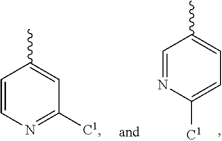

with C¹ being selected from the group consisting of —H, —NH₂,

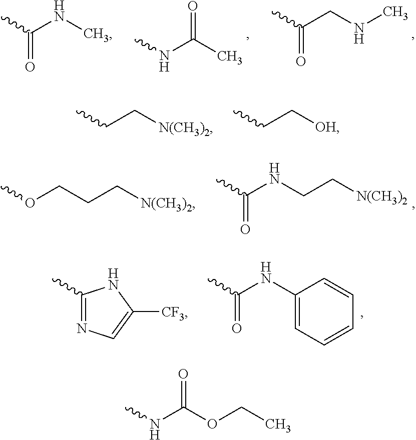

—NH—C(O)—OtBu, —NH—C(O)—N(CH₃)—O(O)—OtBu, —NH—C(O)—CH₂—N(CH₃)—C(O)—OtBu, —NH—C(O)—CH₂—NHCH₃, —NH—C(O)—Obenzyl, —NH—C(O)—CH₂—OH, —NH—(CH₂)₂—N(CH₃)₂, and —NH—S(O)₂—CH₃; preferably from the group consisting of —H,

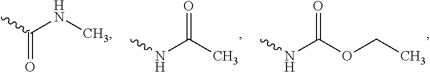

—NH—C(O)—OtBu, —NH—C(O)—N(CH₃)—O(O)—OtBu, —NH—C(O)—CH₂—N(CH₃)—C(O)—OtBu, —NH—C(O)—CH₂—NHCH₃, —NH—C(O)—Obenzyl, and —NH—C(O)—CH₂—OH.

In a particular third aspect, the compound has a structure of formula (I) in which

L is —O—

A is a ring selected from the group consisting of

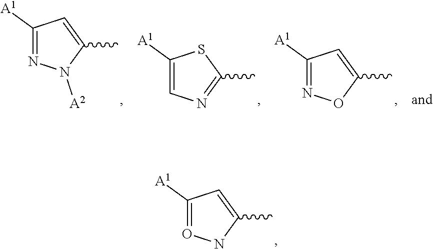

A¹ is tert-butyl,

A² is a phenyl optionally substituted with 1, 2 or 3 substituents selected from the group consisting of —CH₃, —Cl, —F, —CN, —OCH₃, —O—(CH₂)₂-piperidinyl, —O—(CH₂)₂-morpholinyl, —O—(CH₂)₃—N(CH₃)₂, and —CH₂-morpholinyl, or A² is

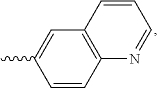

B is a ring system selected in the group consisting of

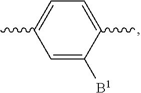

with B¹ being selected from the group consisting of —CF₃, —SCH₂CH₃, —OCH₃, and —OCF₃, preferably —CF₃ or —SCH₂CH₃,

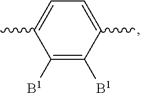

with B¹ being F,

C is selected in the group consisting of

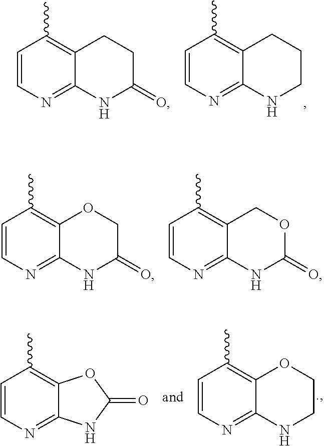

or C is selected from the group consisting of

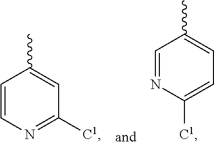

with C¹ being selected from the group consisting of

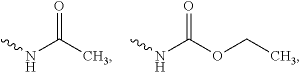

—NH—C(O)—OtBu, —NH—C(O)—CH₂—N(CH₃)—C(O)—OtBu and —NH—C(O)—N(CH₃)—CO—OtBu.

In a particular third aspect, the compound can be selected in the group consisting of
1-(3-(tert-butyl)-1-phenyl-1H-pyrazol-5-yl)-3-(4-((3-oxo-3,4-dihydropyrido[2,3-b]pyrazin-8-yl)oxy)-2-(trifluoromethyl)phenyl)urea;
1-(3-(tert-butyl)-1-(p-tolyl)-1H-pyrazol-5-yl)-3-(4-((3-oxo-3,4-dihydropyrido[2,3-b]pyrazin-8-yl)oxy)-2-(trifluoromethyl)phenyl)urea;
1-(3-(tert-butyl)-1-phenyl-1H-pyrazol-5-yl)-3-(2-(ethylthio)-4-((3-oxo-3,4-dihydropyrido[2,3-b]pyrazin-8-yl)oxy)phenyl)urea;
1-(3-(tert-butyl)-1-phenyl-1H-pyrazol-5-yl)-3-(2,3-difluoro-4-((3-oxo-3,4-dihydropyrido[2,3-b]pyrazin-8-yl)oxy)phenyl)urea; and
1-(3-(tert-butyl)-1-phenyl-1H-pyrazol-5-yl)-3-(2,5-difluoro-4-((3-oxo-3,4-dihydropyrido[2,3-b]pyrazin-8-yl)oxy)phenyl)urea;

or a pharmaceutically acceptable salt thereof.

In a fourth aspect, the compound has a structure of formula (I) in which

A is

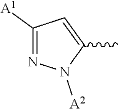

A¹ is selected from the group consisting of methyl, —CF₃, isopropyl, cyclopropyl, isobutyl, sec-butyl, tert-butyl,

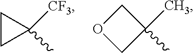

and A² is selected from the group consisting of a phenyl substituted with 1, 2 or 3 substituents selected from the group consisting of methoxy, —Cl, —F, —CN, —O—(CH₂)₂-piperidinyl, —O—(CH₂)₂-morpholinyl, —O—(CH₂)₃—N(CH₃)₂, and —CH₂-morpholinyl; a phenyl or pyridinyl substituted with 2 or 3 methyl, and a pyridinyl substituted with 1, 2 or 3 substituents selected from the group consisting of methyl, —Cl, —F, —CN, —O—(CH₂)₂-piperidinyl, —O—(CH₂)₂-morpholinyl, —O—(CH₂)₃—N(CH₃)₂, and —CH₂-morpholinyl;

B is a ring system selected from the group consisting of

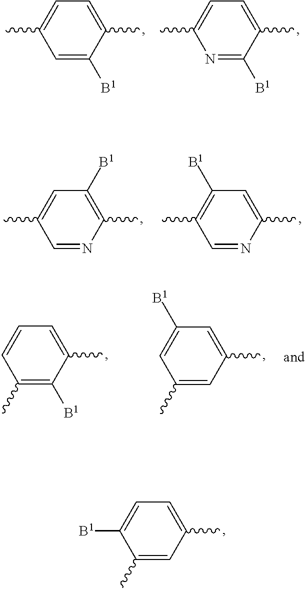

wherein B¹ is —SCH₃,

C is selected from the group consisting of

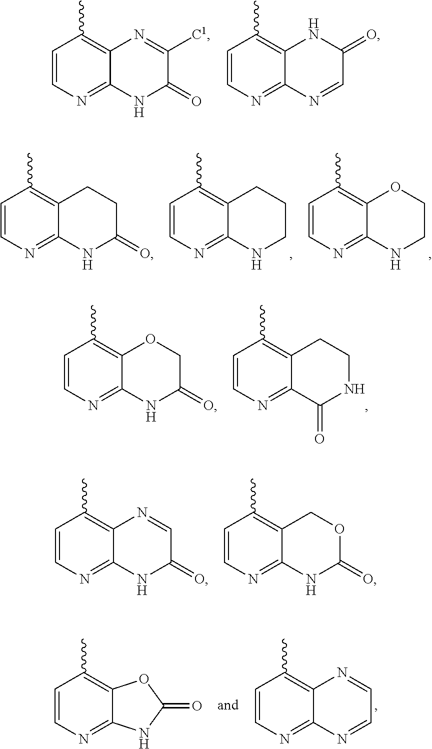

wherein C¹ is selected from the group consisting of —H, —CH₃, —OCH₃, —CN or

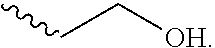

In a particular fourth aspect, the compound has a structure of formula (I) in which

A is

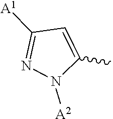

A¹ is tert-butyl,

and A² is selected from the group consisting of a phenyl substituted with 1, 2 or 3 substituents selected from the group consisting of methoxy, —Cl, —F, —CN, —O—(CH₂)₂-piperidinyl, —O—(CH₂)₂-morpholinyl, —O—(CH₂)₃—N(CH₃)₂, and —CH₂-morpholinyl; a phenyl or pyridinyl substituted with 2 or 3 methyl, and a pyridinyl substituted with 1, 2 or 3 substituents selected from the group consisting of methyl, —Cl, —F, —CN, —O—(CH₂)₂-piperidinyl, —O—(CH₂)₂-morpholinyl, —O—(CH₂)₃—N(CH₃)₂, and —CH₂-morpholinyl;

B is a ring system selected from the group consisting of

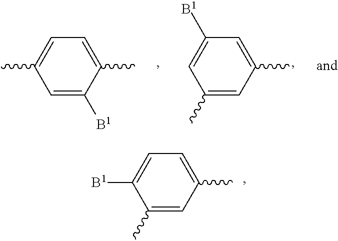

wherein B¹ is —SCH₃,

C is selected from the group consisting of

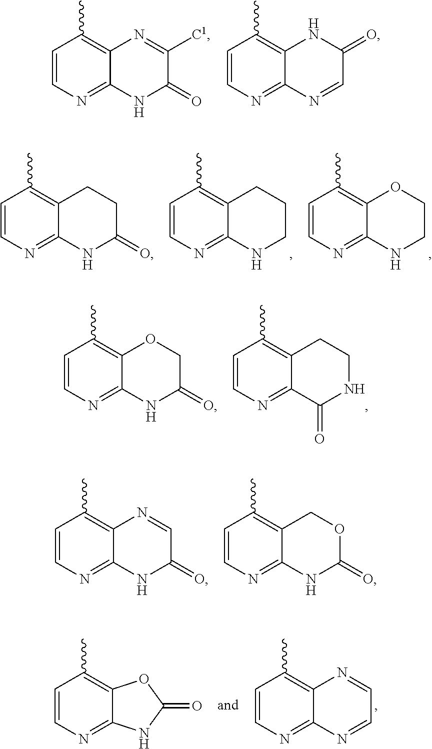

wherein C¹ is selected from the group consisting of —H, —CH₃, —OCH₃, —CN or

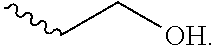

In a particular fourth aspect, the compound has a structure of formula (I) in which

A is

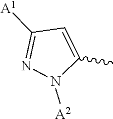

A¹ is tert-butyl,

and A² is selected from the group consisting of a phenyl substituted with 1, 2 or 3 substituents selected from the group consisting of —Cl, —F, —CN, —O—(CH₂)₂-piperidinyl, —O—(CH₂)₃—N(CH₃)₂, and —CH₂-morpholinyl; a pyridinyl substituted with a methyl; and a phenyl substituted by two methyl;

B is

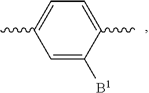

with B¹ being —SCH₃,

C is selected in the group consisting of

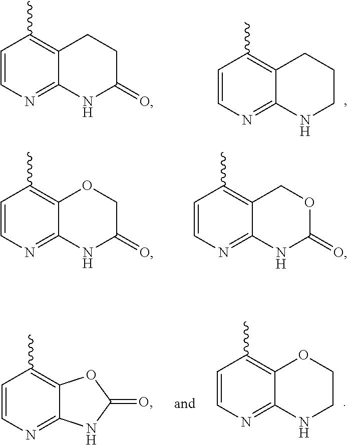

In a particular fourth aspect, the compound can be selected in the group consisting of
1-(3-(tert-butyl)-1-(4-methoxyphenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(3-chlorophenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(3-methoxyphenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(3-fluorophenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(quinolin-6-yl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(4-cyanophenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(3,4-dimethylphenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(4-(2-(piperidin-1-yl)ethoxy)phenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(4-(2-morpholinoethoxy)phenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(3-chloro-4-methylphenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(3-chloro-4-fluorophenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(4-(3-(dimethylamino)propoxy)phenyl)-1H-pyrazol-5-yl)-3-(2-(methylthio)-4-((3-oxo-3,4-dihydropyrido[2,3-b]pyrazin-8-yl)oxy)phenyl)urea; and
1-(3-(tert-butyl)-1-(4-(morpholinomethyl)phenyl)-1H-pyrazol-5-yl)-3-(2-(methylthio)-4-((3-oxo-3,4-dihydropyrido[2,3-b]pyrazin-8-yl)oxy)phenyl)urea;

or a pharmaceutically acceptable salt thereof.

In a fifth aspect, the compound has a structure of formula (I) in which

L is —O—

A is

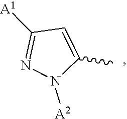

A¹ is selected from the group consisting of methyl, —CF₃, isopropyl, cyclopropyl, isobutyl, sec-butyl, tert-butyl,

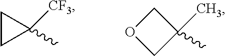

A² is

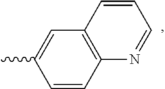

B is a ring system selected from the group consisting of

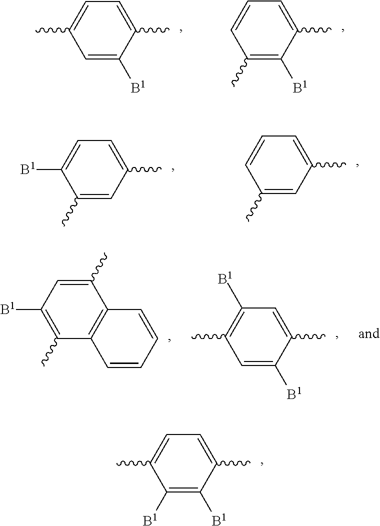

wherein, when B is a phenyl, B¹ is selected from the group consisting of —F, —Cl, —SCH₃, —SCH₂CH₃, —CH₃, —CF₃, —OCH₃, and —OCF₃ and, when B is a naphtyl, B¹ is —H, and

C is selected from the group consisting of

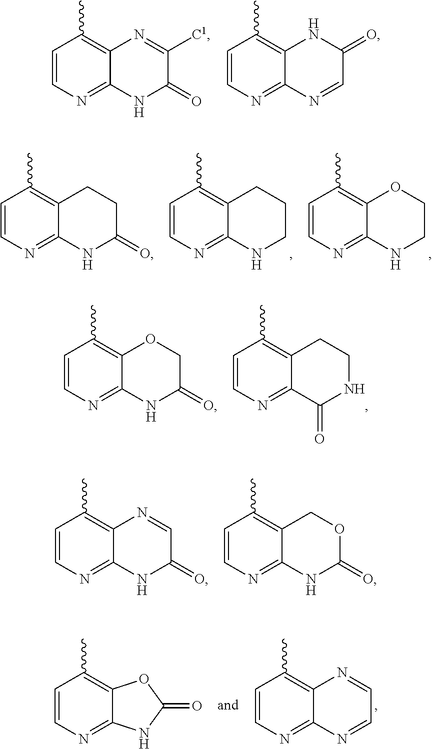

wherein C¹ is selected from the group consisting of —H, —CH₃, —OCH₃, —CN or

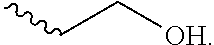

In a particular fifth aspect, the compound has a structure of formula (I) in which

L is —O—

A is

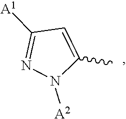

A¹ is tert-butyl,

A² is

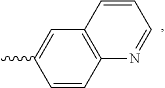

B is a ring system selected from the group consisting of

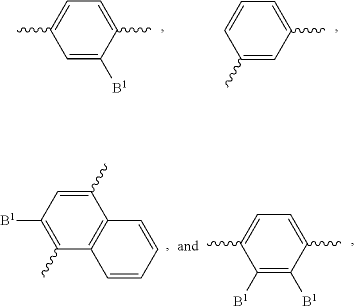

wherein, when B is a phenyl, B¹ is selected from the group consisting of —F, —Cl, —SCH₃, —SCH₂CH₃, —CH₃, —CF₃, —OCH₃, and —OCF₃ and, when B is a naphtyl, B¹ is —H, and

C is selected from the group consisting of

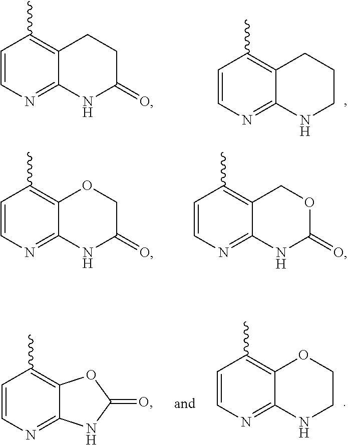

In a particular fifth aspect, the compound can be selected in the group consisting of
1-(3-(tert-butyl)-1-(quinolin-6-yl)-1H-pyrazol-5-yl)-3-(2-fluoro-4-((3-oxo-3,4-dihydropyrido[2,3-b]pyrazin-8-yl)oxy)phenyl)urea;
1-(3-(tert-butyl)-1-(quinolin-6-yl)-1H-pyrazol-5-yl)-3-(4-((3-oxo-3,4-dihydropyrido[2,3-b]pyrazin-8-yl)oxy)naphthalen-1-yl)urea; and
1-(3-(tert-butyl)-1-(quinolin-6-yl)-1H-pyrazol-5-yl)-3-(2-(methylthio)-4-((3-oxo-3,4-dihydropyrido[2,3-b]pyrazin-8-yl)oxy)phenyl)urea;

or a pharmaceutically acceptable salt thereof.

In a sixth aspect, the compound has a structure of formula (I) in which

L is —O—

A is a ring selected from the group consisting of

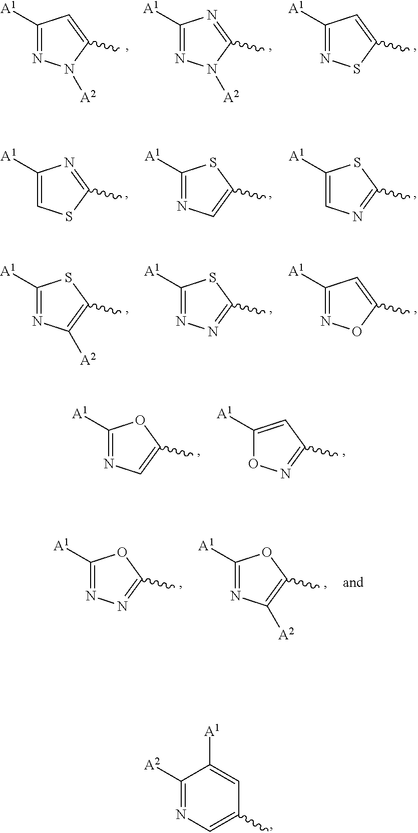

A¹ is selected from the group consisting of methyl, —CF₃, isopropyl, cyclopropyl, isobutyl, sec-butyl, tert-butyl,

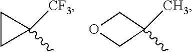

and A² is selected from the group consisting of H, methyl, iso-propyl, benzyl, phenyl, chlorine,

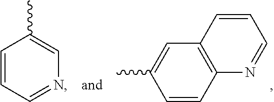

wherein A¹ and A² are optionally fluorinated, and A² is optionally substituted with 1, 2 or 3 substituents selected from the group consisting of C₁-C₄ alkyl, —Cl, —F, —CN, —OR^{A}, and —SO₂R^{A}, wherein R^{A} is C₁-C₁₀ alkyl, and the substituent being optionally substituted by C₃-C₇ heterocycloalkyl or NRR, with R being a C₁-C₃ alkyl,

B is a ring system selected from the group consisting of

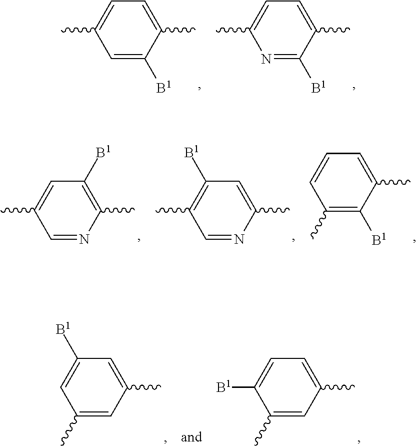

wherein B¹ is —SCH₃,

C is selected from the group consisting of

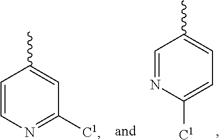

with C¹ being selected C¹ is selected from the group consisting of wherein C¹ is selected from the group consisting of —H, —NH₂, —NR¹, —C(O)—NH—R¹, —NH—C(O)—R¹, —NH—S(O)₂—R¹, —NH—C(O)—OR¹, —C(O)—R¹, —R¹, —OR¹, —SO₂R¹, with R¹ being selected from the group consisting of C₁-C₁₀ alkyl, C₆-C₁₄ aryl, C₃-C₁₃ heteroaryl, and C₇-C₂₄ alkaryl, wherein each hydrogen in each of these groups may be independently replaced by a group selected from a halogen, a hydroxy, —OR′, and —NR′R″, each hydrogen in each of R′ and R″ may be independently replaced by —COR′″ or COOR′″, wherein R′, R″ and R′″ are independently a C₁-C₆ alkyl.

In a particular sixth aspect, the compound has a structure of formula (I) in which

L is —O—

A is a ring selected from the group consisting of

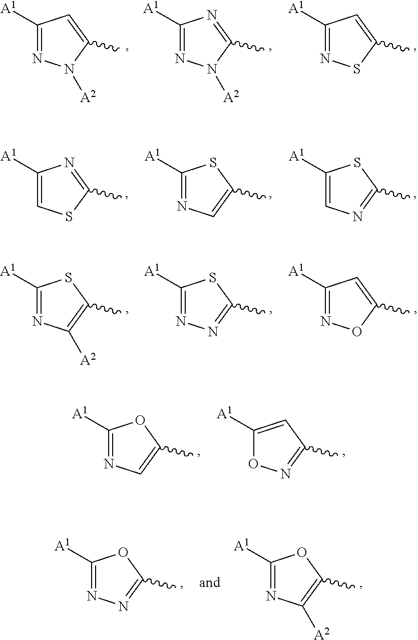

A¹ is tert-butyl,

A² is a phenyl or

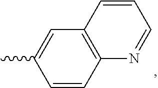

optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,

B is a ring system selected from the group consisting of

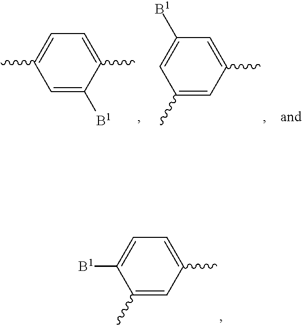

wherein B¹ is —SCH₃,

C is selected from the group consisting of

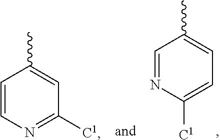

with C¹ being selected from the group consisting of —H, —NH₂,

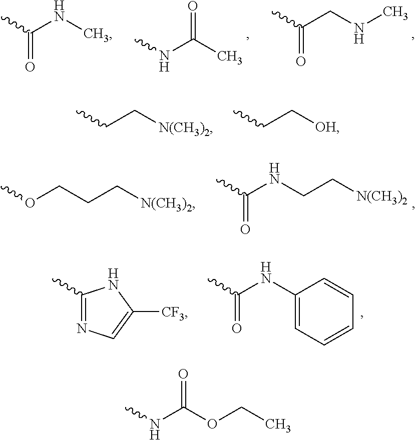

—NH—C(O)—OtBu, —NH—C(O)—N(CH₃)—C(O)—OtBu, —NH—C(O)—CH₂—N(CH₃)—C(O)—OtBu, —NH—C(O)—CH₂—NHCH₃, —NH—C(O)—Obenzyl, —NH—C(O)—CH₂—OH, —NH—(CH₂)₂—N(CH₃)₂, and —NH—S(O)₂—CH₃; preferably from the group consisting of —H,

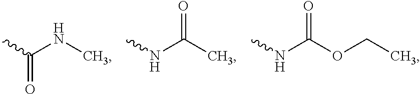

—NH—C(O)—OtBu, —NH—C(O)N(CH₃)—C(O)—OtBu, —NH—C(O)—CH₂—N(CH₃)—C(O)—OtBu, —NH—C(O)—CH₂—NHCH₃, —NH—C(O)—Obenzyl, and —NH—C(O)—CH₂—OH.

In a particular sixth aspect, the compound has a structure of formula (I) in which

L is —O—

A is a ring selected from the group consisting of

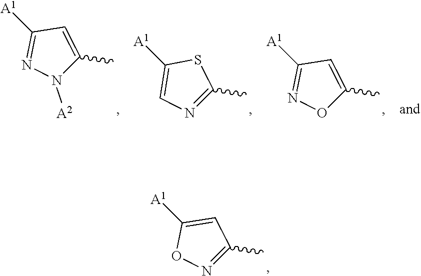

A¹ is tert-butyl,

A² is a phenyl optionally substituted with 1, 2 or 3 substituents selected from the group consisting of methyl, —Cl, —F, —CN, methoxy, —O—(CH₂)₂-piperidinyl, —O—(CH₂)₂-morpholinyl, —O—(CH₂)₃—N(CH₃)₂, and —CH₂-morpholinyl, or A² is

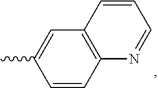

B is

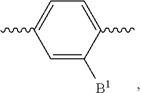

with B¹ being —SCH₃,

C is selected from the group consisting of

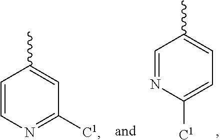

with C¹ being selected from the group consisting of

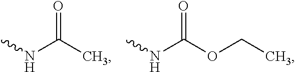

—NH—C(O)—OtBu, —NH—C(O)—CH₂—N(CH₃)—C(O)—OtBu and —NH—C(O)—N(CH₃)—CO—OtBu.

In a particular sixth aspect, the compound can be selected in the group consisting of
5-(4-(3-(3-(tert-butyl)-1-phenyl-1H-pyrazol-5-yl)ureido)-3-(methylthio)phenoxy)-N-methylnicotinamide;
N-(4-(4-(3-(3-(tert-butyl)-1-phenyl-1H-pyrazol-5-yl)ureido)-3-(methylthio)phenoxy)pyridin-2-yl)acetamide;
tert-butyl (4-(4-(3-(3-(tert-butyl)-1-phenyl-1H-pyrazol-5-yl)ureido)-3-(methylthio)phenoxy)pyridin-2-yl)carbamate;
tert-butyl (2-((4-(4-(3-(3-(tert-butyl)-1-phenyl-1H-pyrazol-5-yl)ureido)-3-(methylthio)phenoxy)pyridin-2-yl)amino)-2-oxoethyl)(methyl)carbamate;
N-(4-(4-(3-(3-(tert-butyl)-1-phenyl-1H-pyrazol-5-yl)ureido)-3-(methylthio)phenoxy)pyridin-2-yl)-2-(methylamino)acetamide;
1-(3-(tert-butyl)-1-phenyl-1H-pyrazol-5-yl)-3-(4-((2-((2-(dimethylamino)ethyl)amino)pyridin-4-yl)oxy)-2-(methylthio)phenyl)urea hydrochloride;
ethyl (4-(4-(3-(3-(tert-butyl)-1-phenyl-1H-pyrazol-5-yl)ureido)-3-(methylthio)phenoxy)pyridin-2-yl)carbamate;
benzyl (4-(4-(3-(3-(tert-butyl)-1-phenyl-1H-pyrazol-5-yl)ureido)-3-(methylthio)phenoxy)pyridin-2-yl)carbamate;
ethyl (4-(4-(3-(3-(tert-butyl)isoxazol-5-yl)ureido)-3-(methylthio)phenoxy)pyridin-2-yl)carbamate;
ethyl (4-(4-(3-(5-(tert-butyl)thiazol-2-yl)ureido)-3-(methylthio)phenoxy)pyridin-2-yl)carbamate; and
ethyl (4-(4-(3-(5-(tert-butyl)isoxazol-3-yl)ureido)-3-(methylthio)phenoxy)pyridin-2-yl)carbamate;

or a pharmaceutically acceptable salt thereof.

In a particular seventh aspect, the compound has a structure of formula (I) in which

A is a ring system selected from the group consisting of

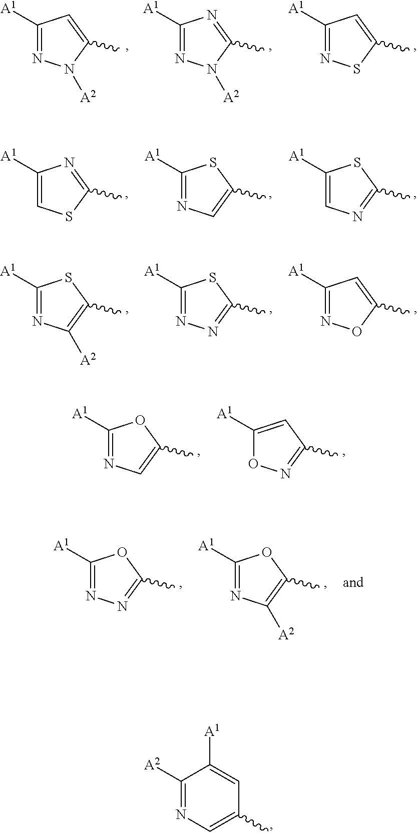

A¹ is selected from the group consisting of methyl, —CF₃, isopropyl, cyclopropyl, isobutyl, sec-butyl, tert-butyl,

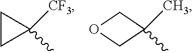

and A² is selected from the group consisting of H, methyl, iso-propyl, benzyl, phenyl, chlorine,

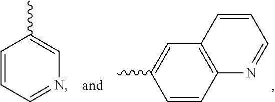

wherein A¹ and A² are optionally fluorinated, and A² is optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,

B is a ring system selected from the group consisting of

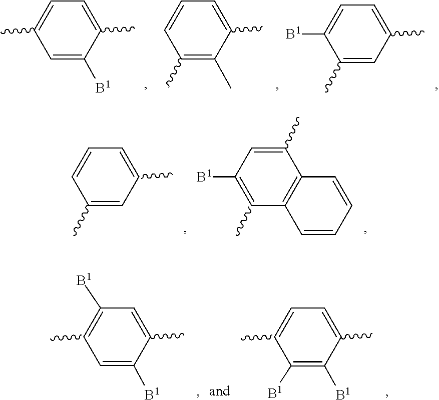

wherein, when B is a phenyl, B¹ is selected from the group consisting of —F, —Cl, —SCH₃, —SCH₂CH₃, —CH₃, —CF₃, —OCH₃, and —OCF₃ and, when B is a naphtyl, B¹ is —H, and

C is selected from the group consisting of

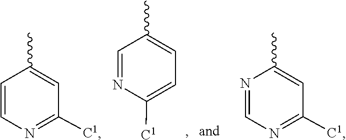

wherein C¹ is selected from the group consisting of —NH—C(O)R¹ or —NH—C(O)—OR¹ with R¹ being selected from the group consisting of C₁-C₁₀ alkyl, C₆-C₁₄ aryl, C₃-C₁₃ heteroaryl, and C₇-C₂₄ alkaryl, wherein each hydrogen in each of these groups may be independently replaced by a group selected from halogen, hydroxy, —OR′, and —NR′R each hydrogen in each of R′ and R″ may be independently replaced by —COR′″ or COOR′″, wherein R′, R″ and R′″ are independently a C₁-C₆ alkyl.

In a particular seventh aspect, the compound has a structure of formula (I) in which

L is —O—

A is a ring selected from the group consisting of

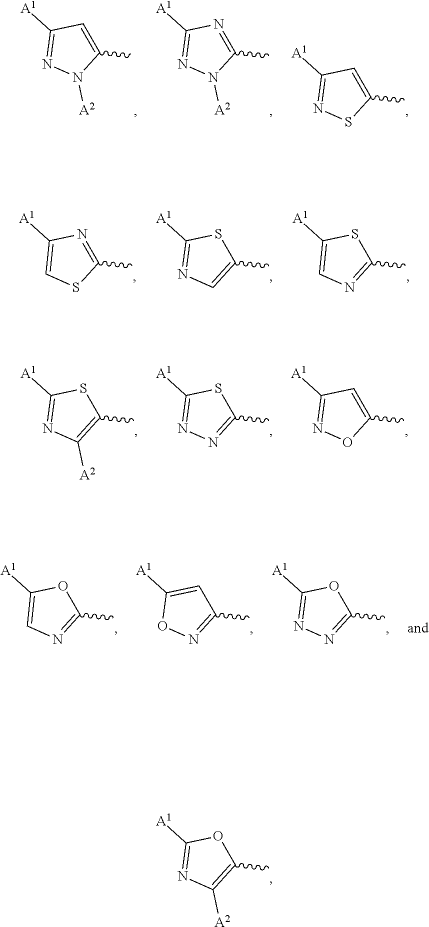

A¹ is tert-butyl,

A² is a phenyl or

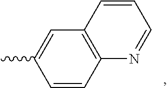

optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,

B is a ring system selected from the group consisting of

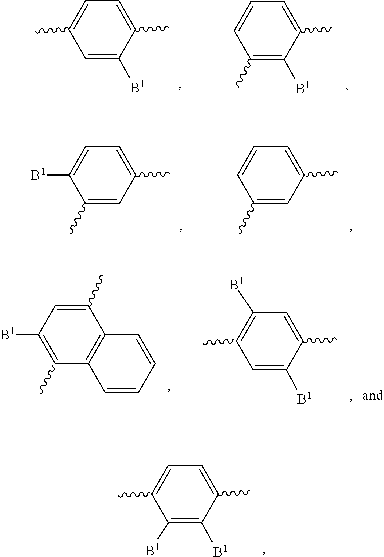

wherein, when B is a phenyl, B¹ is selected from the group consisting of —F, —SCH₃, —SCH₂CH₃, —CF₃, —OCH₃, and —OCF₃ and, when B is a naphtyl, B¹ is —H, and

C is selected from the group consisting of

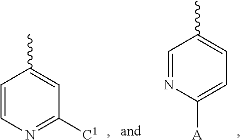

with C¹ being selected from the group consisting of

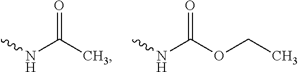

—NH—C(O)—OtBu, —NH—C(O)—N(CH₃)—C(O)—OtBu, —NH—C(O)—CH₂—N(CH₃)—C(O)—OtBu, —NH—C(O)—CH₂—NHCH₃, —NH—C(O)—Obenzyl, —NH—C(O)—CH₂—OH, preferably

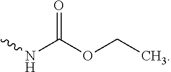

In a particular seventh aspect, the compound has a structure of formula (I) in which

L is —O—

A is a ring selected from the group consisting of

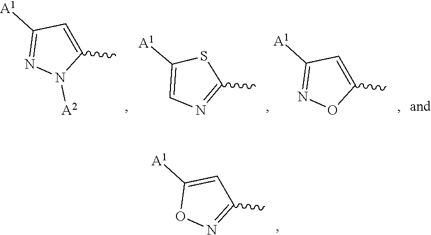

A¹ is tert-butyl,

A² is a phenyl optionally substituted with 1, 2 or 3 substituents selected from the group consisting of methyl, —Cl, —F, —CN, methoxy, —O—(CH₂)₂-piperidinyl, —O—(CH₂)₂-morpholinyl, —O—(CH₂)₃—N(CH₃)₂, and —CH₂-morpholinyl, or A² is

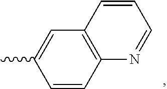

B is a ring system selected in the group consisting of

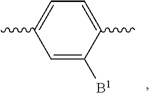

with B¹ being selected from the group consisting of —F, —SCH₃, —SCH₂CH₃, —CF₃, —OCH₃, and —OCF₃, preferably —SCH₃,

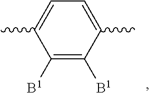

with B¹ being F,

and

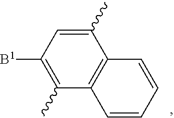

with B¹ being —H, and

C is selected in the group consisting of

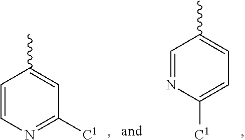

with C¹ being selected from the group consisting of

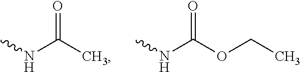

—NH—C(O)—OtBu, —NH—C(O)—N(CH₃)—C(O)—OtBu, —NH—C(O)—CH₂—N(CH₃)—C(O)—OtBu, —NH—C(O)—CH₂—NHCH₃, —NH—C(O)—Obenzyl, —NH—C(O)—CH₂—OH, preferably

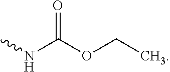

In a particular seventh aspect, the compound can be selected in the group consisting of
N-(4-(4-(3-(3-(tert-butyl)-1-phenyl-1H-pyrazol-5-yl)ureido)-3-fluorophenoxy)pyridin-2-yl)acetamide;
N-(4-(4-(3-(3-(tert-butyl)-1-phenyl-1H-pyrazol-5-yl)ureido)-3-(methylthio)phenoxy)pyridin-2-yl)acetamide;
tert-butyl (4-(4-(3-(3-(tert-butyl)-1-phenyl-1H-pyrazol-5-yl)ureido)-3-(methylthio)phenoxy)pyridin-2-yl)carbamate;
tert-butyl (2-((4-(4-(3-(3-(tert-butyl)-1-phenyl-1H-pyrazol-5-yl)ureido)-3-(methylthio)phenoxy)pyridin-2-yl)amino)-2-oxoethyl)(methyl)carbamate;
ethyl (4-(4-(3-(3-(tert-butyl)-1-phenyl-1H-pyrazol-5-yl)ureido)-3-(methylthio)phenoxy)pyridin-2-yl)carbamate
ethyl (4-((4-(3-(3-(tert-butyl)-1-phenyl-1H-pyrazol-5-yl)ureido)naphthalen-1-yl)oxy)pyridin-2-yl)carbamate;
ethyl (4-((4-(3-(3-(tert-butyl)isoxazol-5-yl)ureido)naphthalen-1-yl)oxy)pyridin-2-yl)carbamate;
ethyl (4-((4-(3-(5-(tert-butyl)thiazol-2-yl)ureido)naphthalen-1-yl)oxy)pyridin-2-yl)carbamate;
ethyl (4-(4-(3-(3-(tert-butyl)isoxazol-5-yl)ureido)-3-(methylthio)phenoxy)pyridin-2-yl)carbamate;
ethyl (4-(4-(3-(5-(tert-butyl)thiazol-2-yl)ureido)-3-(methylthio)phenoxy)pyridin-2-yl)carbamate;
benzyl (4-(4-(3-(3-(tert-butyl)-1-phenyl-1H-pyrazol-5-yl)ureido)-3-(methylthio)phenoxy)pyridin-2-yl)carbamate;
N-(4-(4-(3-(3-(tert-butyl)-1-phenyl-1H-pyrazol-5-yl)ureido)-3-(methylthio)phenoxy)pyridin-2-yl)-2-hydroxyacetamide; and
tert-butyl (4-(4-(3-(3-(tert-butyl)-1-phenyl-1H-pyrazol-5-yl)ureido)-3-(methylthio)phenoxy)pyridin-2-yl)(2-(dimethylamino)ethyl)carbamate;

or a pharmaceutically acceptable salt thereof.

### BRIEF DESCRIPTION OF THE FIGURES

FIG. 1—the workflow of the myotube assay, aiming at monitoring myogenic activity of compounds and effect on satellite-like cells.

FIG. 2—the myogenic activity of compound (i) in a dose-response assay, as shown by an increase of the total myotube surface/well upon increase of compound concentration.

FIG. 3—the positive effect on satellite-like cells of compound (i) in a dose-response assay, as shown by the increase of Pax7-positive cell percentage upon increase of compound concentration.

FIG. 4—representative images for the dose-response of the total myotube area readout for compound (i), corresponding to FIG. 2.

FIG. 5—representative images for the dose-response of the percentage of Pax7-positive cell readout for compound (i), corresponding to FIG. 3.

### DETAILED DESCRIPTION

### Compounds

In a first aspect, the invention provides a compound, especially for use in treating, ameliorating, delaying, curing and/or preventing a disease or condition associated with muscle cells and/or satellite cells, or a pharmaceutically acceptable salt thereof, wherein said compound is represented by structure (I),

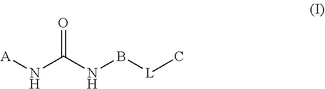

wherein A, B and C are ring systems comprising a five-membered or a six-membered aromatic or heteroaromatic ring, wherein said ring systems do not comprise more than 30 carbon atoms;

wherein L is selected from the group consisting of —O—, —S—, —NR—, —(CH₂)ₘ—, —C(O)—, —CH(OH)—, —(CH₂)ₘO—, —(CH₂)ₘS—, —(CH₂)ₘNR—, —O(CH₂)ₘ—, —CHX—, —CX₂—, —S(CH₂)ₘ— and —NR(CH₂)ₘ—, wherein m is 1, 2 or 3, X is a halogen, R is selected from the group consisting of H, C₁-C₁₀ alkyl, C₂-C₁₀ alkenyl, C₃-C₁₀ cycloalkyl, C₆-C₁₄ aryl, C₃-C₁₃ heteroaryl and C₇-C₂₄ alkaryl, wherein R is optionally substituted with one or more halogen substituents, and L is optionally substituted. Preferably, L is —O—.

A compound as defined in this first aspect is referred to in the current application as “a compound according to the invention” or as “a compound of the invention”. Said terms are used interchangeably in the context of this application.

In the context of this application, a single compound according to the invention is referred to by a letter between parentheses; whereas a Markush formula encompassing several compounds according to the invention is referred to by a roman numeral between parentheses. Intermediates in the synthesis of compounds according to the invention are referred to by Arabic numbers, or dot-separated combinations thereof.

The five-membered or six-membered aromatic or heteroaromatic ring in a ring system A, B or C of a compound according to the invention may be comprised in a larger aromatic or heteroaromatic system. For example, A may be naphtyl, which comprises a six-membered aromatic ring, which is part of an aromatic bicyclic ring system comprising 10 carbon atoms. It is also understood that said five-membered or six-membered aromatic or heteroaromatic rings may be substituted.

A ring system A, B or C comprised in a compound according to the invention may comprise additional cyclic structures, besides said five-membered or a six-membered aromatic or heteroaromatic ring. Such additional cyclic structures may be for example cycloalkyl, heterocycloalkyl, aryl or heteroaryl structures.

L may be optionally substituted. By this is meant that every hydrogen atom in L may independently be substituted by a substituent selected from the group consisting of halogens, —CN, —CO₂R^{L}, —C(O)R^{L}, C(O)NR^{L}R^{L}, —NO₂, —OR^{L}, —SR^{L}, —NR^{L}R^{L}, —NR^{L}C(O)R^{L}, —NR^{L}C(O)OR^{L}, C₁-C₁₀ alkyl, C₂-C₁₀ alkenyl, C₂-C₁₀ alkynyl, alkoxy, C₃-C₁₀ cycloalkyl, C₃-C₇ heterocycloalkyl, C₆-C₁₄ aryl, C₇-C₂₄ alkaryl, C₃-C₁₃ heteroaryl and C₄-C₂₃ alkheteroaryl, preferably by a halogen. Herein, R^{L} is independently selected from the group consisting of C₁-C₁₀ alkyl, C₂-C₁₀ alkenyl, C₃-C₁₀ cycloalkyl, C₆-C₁₄ aryl, C₃-C₁₃ heteroaryl and C₇-C₂₄ alkaryl, wherein each hydrogen in each of these groups may be independently substituted by a halogen.

The synthesis of a compound according to invention is described in detail in example 1.

In a preferred embodiment is provided a compound according to the invention, wherein ring systems A, B and C are independently selected from the group consisting of phenyl, pyridinyl, naphthyl, pyrimidinyl, benzothiazoyl, quinoline, isoquinoline, phthalimidinyl, diphenyl ether(phenyloxyphenyl), diphenylthioether(phenylthiophenyl), diphenylamine(phenylaminophenyl), phenylpyridinyl, ether(pyridinyloxyphenyl), pyridinylmethylphenyl, phenylpyridinyl thioether(pyridinylthiophenyl), pyridinylmethylphenyl, phenylpyridinylthioether(pyridinylthiophenyl), phenylbenzothiazolyl ether(benzothiazolyloxyphenyl), phenylbenzothiazolyl thioether(benzothiazolylthiophenyl), phenylpyridinyl ether, phenylquinoline thioether, phenylnaphthyl ether, pyridinylnaphthyl ether, pyridinylnaphthyl thioether, and phthalimidylmethylphenyl, acridinyl, azepinyl, benzimidazolyl, benzindolyl, benzisothiazolyl, 1,3-benzodioxolyl, benzothiadizolyl, benzo[b][1,4]dioxepinyl, benzo[b][1,4]oxazinyl, 1,4-benzodioxazinyl, benzodioxolyl, benzodioxinyl, benzofuryl, benzofuranonyl, benzonaphthofuranyl, benzopyranyl, benzopyranonyl, benzopyrazolyl, benzothienyl, benzoxazolyl, benzisoxazolyl, benzothiazolyl, benzothieno[3,2-d]pyrimidinyl, benz-1,3-oxadiazolyl, benzotriazolyl, benzo[4,6]imidazo[1,2-a]pyridinyl, carbazolyl, cinnonyl, cyclopenta[d]pyrimidinyl, 6,7-dihydro-5H-cyclopenta[4,5]thieno[2,3-d]pyrimidinyl, 2,3-dihydro-1H-indenyl, 2,3-dihydrobenzo[b][1,4]oxathiine, 3,4-dihydro-pyrido[3,2-b][1,4]oxazine, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, imidazolyl, 1H-imidazo[4,5-b]pyridinonyl, imidazo[4,5-b]pyridinyl, indazolyl, indolinyl, indolizinyl, indolyl, isoindolinyl, isoindolyl, isoquinolyl, isothiazolo[3,4-b]pyridinyl, isothiazolo[5,4-b]pyridinyl, isothiazolyl, isoxazolo[5,4-b]pyridinyl, isoxazolyl, 5,8-methano-5,6,7,8-tetrahydroquinazolinyl, naphthyridinyl, 1,6-naphthyridinonyl, 1,8-naphthyridinonyl, 5,6,6a,7,8,9,10,10a-octahydrobenzo[h]quinazolinyl, oxadiazolyl, oxazolo[4,5-b]pyridinyl, oxazolyl, oxiranyl, 2-oxoazepinyl, 1-phenyl-1H-pyrrolyl, phenazinyl, phenothiazinyl, phenoxazinyl, phthalazinyl, pteridinonyl, pteridinyl, purinyl, pyrazinyl, pyrazolo[3,4-b]pyridinyl, pyrazolo[3,4-d]pyrimidinyl, pyrazolyl, pyridazinyl, pyrido[3,2-d]pyrimidinonyl, pyrido[2,3-d]pyrimidinyl, pyrido[3,2-d]pyrimidinyl, pyrido[3,4-d]pyrimidinyl, pyrido[2,3-b]pyrazinonyl, pyrido[2,3-b]pyrazinyl, pyrido[2,3-e][1,2,4]triazinonyl, pyridyl, pyrimidinyl, pyrrolo[2,3-b]pyridinonyl, pyrrolo[2,3-b]pyridinyl, pyrrolyl, quinazolinyl, quinolyl, quinoxalinonyl, quinoxalinyl, 6,7,8,9-tetrahydro-5H-cyclohepta[4,5]thieno[2,3-d]pyrimidinyl, 1,2,3,4-tetrahydronaphthalenyl, 1,2,3,4-tetrahydro-1,8-naphthyridinyl, 5,6,7,8-tetrahydro-1,8-naphthyridinonyl, 5,6,7,8-tetrahydrobenzo[4,5]thieno[2,3-d]pyrimidinyl, 5,6,7,8-tetrahydropyrido[4,5-c]pyridazinyl, 5,6,7,8-tetrahydroquinazolinyl, tetrahydroquinolinyl, tetrazolyl, [1,2,5]thiadiazolo[3,4-b]pyridinyl, thiadiazolyl, thiazolo[4,5-b]pyridinyl, thiazolo[5,4-b]pyridinyl, thiazolyl, thieno[2,3-d]pyrimidinyl, thieno[3,2-d]pyrimidinyl, thieno[2,3-c]pyridinyl, thienyl, thiopyranyl, triazinyl [1,2,3]triazolo[4,5-b]pyridinyl and triazolyl, wherein said ring systems are optionally substituted, as defined below.

In an alternative preferred embodiment is provided a compound according to the invention, wherein ring systems A, B and C are independently selected from the group consisting of phenyl, pyridinyl, naphthalenyl, pyrimidinyl, quinolinyl, isoquinolinyl, phthalimidinyl, diphenyl ether(phenyloxyphenyl), diphenyl thioether(phenylthiophenyl), diphenyl amine(phenylaminophenyl), phenylpyridinyl, ether(pyridinyloxyphenyl), pyridinylmethylphenyl, phenylpyridinyl thioether(pyridinylthiophenyl), pyridinylmethylphenyl, phenylpyridinylthioether(pyridinylthiophenyl), phenylbenzothiazolyl ether(benzothiazolyloxyphenyl), phenylbenzothiazolyl thioether(benzothiazolylthiophenyl), phenylpyridinyl ether, phenylquinolinyl thioether, phenylnaphthyl ether, pyridinylnaphthyl ether, pyridinylnaphthyl thioether, and phthalimidylmethylphenyl, acridinyl, azepinyl, benzimidazolyl, benzindolyl, benzisothiazolyl, 1,3-benzodioxolyl, benzothiadizolyl, benzo[b][1,4]dioxepinyl, benzo[b][1,4]oxazinyl, 1,4-benzodioxazinyl, benzodioxolyl, benzodioxinyl, benzofuryl, benzofuranonyl, benzonaphthofuranyl, benzopyranyl, benzopyranonyl, benzopyrazolyl, benzothienyl, benzoxazolyl, benzisoxazolyl, benzothiazolyl, benzothieno[3,2-d]pyrimidinyl, benz-1,3-oxadiazolyl, benzotriazolyl, benzo[4,6]imidazo[1,2-a]pyridinyl, carbazolyl, cinnonyl, cyclopenta[d]pyrimidinyl, dibenzofuranyl, dibenzothiophenyl, 6,7-dihydro-5H-cyclopenta[4,5]thieno[2,3-d]pyrimidinyl, 2,3-dihydro-1H-indenyl, 2,3-dihydrobenzo[b][1,4]oxathiinyl, 3,4-dihydro-1,8-naphthyridinonyl, 3,4-dihydro-pyrido[3,2-b][1,4]oxazinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, 2,2-dioxido-3,4-dihydro-1H-pyrido[2,3-c][1,2]thiazinyl, 2,2-dioxido-1H-pyrido[2,3-c][1,2]thiazinyl, 2,2-dioxido-1H-pyrido[2,3-e][1,3,4]oxathiazinyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, imidazolyl, 1H-imidazo[4,5-b]pyridinonyl, imidazo[4,5-b]pyridinyl, indazolyl, indolinyl, indolizinyl, indolyl, isoindolinyl, isoindolyl, isoquinolyl, isothiazolo[3,4-b]pyridinyl, isothiazolo[5,4-b]pyridinyl, isothiazolyl, isoxazolo[5,4-b]pyridinyl, isoxazolyl, 5,8-methano-5,6,7,8-tetrahydroquinazolinyl, naphthyridinyl, 1,6-naphthyridinonyl, 1,8-naphthyridinonyl, 5,6,6a,7,8,9,10,10a-octahydrobenzo[h]quinazolinyl, oxadiazolyl, oxazolo[4,5-b]pyridinyl, oxazolyl, oxiranyl, 2-oxoazepinyl, 1-phenyl-1H-pyrrolyl, phenazinyl, phenothiazinyl, phenoxazinyl, phthalazinyl, pteridinonyl, pteridinyl, purinyl, pyrazinyl, pyrazolo[3,4-b]pyridinyl, pyrazolo[3,4-d]pyrimidinyl, pyrazolyl, pyridazinyl, pyrido[3,2-b][1,4]oxazinonyl, pyrido[3,2-d]pyrimidinonyl, pyrido[2,3-d]pyrimidinyl, pyrido[3,2-d]pyrimidinyl, pyrido[3,4-d]pyrimidinyl, pyrido[2,3-b]pyrazinonyl, pyrido[2,3-b]pyrazinyl, pyrido[2,3-e][1,2,4]triazinonyl, 7H-pyrrolo[2,3-b]pyridinyl, pyrrolyl, quinazolinyl, quinoxalinonyl, quinoxalinyl, 6,7,8,9-tetrahydro-5H-cyclohepta[4,5]thieno[2,3-d]pyrimidinyl, 1,2,3,4-tetrahydronaphthalenyl, 1,2,3,4-tetrahydro-1,8-naphthyridinyl, 5,6,7,8-tetrahydro-1,8-naphthyridinyl, 8-oxo-6,7,8,9-tetrahydro-5H-pyrido[2,3-b]azepinyl, 3-oxo-3,4-dihydro-2H-pyrido[3,2-b][1,4]oxazinyl, 6-oxo-6,7-dihydro-5H-pyrrolo[2,3-b]pyridinyl, 2-oxo-1,2,4,5-tetrahydropyrido[2,3-d][1,3]oxazepinyl, 2-oxo-1,2,3,5-tetrahydropyrido[2,3-e][1,4]oxazepinyl, 2-oxo-2,4-dihydro-1H-pyrido[2,3-d][1,3]oxazinyl, 4-oxo-2,3,4,5-tetrahydropyrido[3,2-b][1,4]oxazepinyl, 7-oxo-5,6,7,8-tetrahydro-1,8-naphthyridinyl, 2-oxo-1,2,3,4-tetrahydropyrido[2,3-d]pyrimidinyl, 5,6,7,8-tetrahydrobenzo[4,5]thieno[2,3-d]pyrimidinyl, 5,6,7,8-tetrahydropyrido[4,5-c]pyridazinyl, 5,6,7,8-tetrahydroquinazolinyl, tetrahydroquinolinyl, tetrazolyl, [1,2,5]thiadiazolo[3,4-b]pyridinyl, thiadiazolyl, thiazolo[4,5-b]pyridinyl, thiazolo[5,4-b]pyridinyl, thiazolyl, thieno[2,3-d]pyrimidinyl, thieno[3,2-d]pyrimidinyl, thieno[2,3-c]pyridinyl, thienyl, thiopyranyl, triazinyl [1,2,3]triazolo[4,5-b]pyridinyl and triazolyl, wherein said ring systems are optionally substituted, as defined below.

In the context of this preferred embodiment, each hydrogen if ring systems A, B and C may be independently replaced by a substituent selected from the group consisting of halogens, —CN, —CO₂R^{ABC}, —C(O)R^{ABC}, C(O)NR^{ABC}R^{ABC}, —NO₂, C₀-C₁₀ alkOR^{ABC}, in particular —OR^{ABC}, C₀-C₁₀ alkSR^{ABC}, in particular —SR^{ABC}, C₀-C₁₀ alkNR^{ABC}R^{ABC}, in particular —NR^{ABC}R^{ABC}, —NR^{ABC}C(O)R^{ABC}, —NR^{ABC}C(O)OR^{ABC}, —NR^{ABC}C(O)NR^{ABC}R^{ABC}, NR^{ABC}C(NH)NR^{ABC}R^{ABC}, NR^{ABC}S(O)₂R^{ABC}, —NR^{ABC}S(O)₂OR^{ABC}, C₁-C₁₀ alkyl, C₂-C₁₀ alkenyl, C₂-C₁₀ alkynyl, alkoxy, C₃-C₁₀ cycloalkyl, C₄-C₂₀ alkcycloalkyl, C₃-C₇ heterocycloalkyl, C₄-C₁₇ alkheterocycloalkyl, C₆-C₁₄ aryl, C₇-C₂₄ alkaryl, C₃-C₁₃ heteroaryl and C₄-C₂₃ alkheteroaryl; wherein seach hydrogen atoms in each of these groups may be independently replaced by a substituent selected from the group consisting of halogens, —CN, —CO₂R^{ABC}, —C(O)R^{ABC}, C(O)NR^{ABC}R^{ABC}, —NO₂, —OR^{ABC}, —SR^{ABC}, —NR^{ABC}R^{ABC}, —NR^{ABC}C(O)R^{ABC}, —NR^{ABC}C(O)OR^{ABC}, C₁-C₁₀ alkyl, C₂-C₁₀ alkenyl, C₂-C₁₀ alkynyl, alkoxy, C₃-C₁₀ cycloalkyl, C₄-C₂₀ alkcycloalkyl, C₃-C₇ heterocycloalkyl, C₄-C₁₇ alkheterocycloalkyl, C₆-C₁₄ aryl, C₇-C₂₄ alkaryl, C₃-C₁₃ heteroaryl, C₄-C₂₃ alkheteroaryl, C₃-C₇ heterocycloalkyl and C₄-C₁₇ alkheterocycloalkyl; wherein R^{ABC} is selected independently from the group consisting of hydrogen, C₁-C₁₀ alkyl, C₂-C₁₀ alkenyl, C₃-C₁₀ cycloalkyl, C₆-C₁₄ aryl, C₃-C₁₃ heteroaryl, C₇-C₂₄ alkaryl, wherein each hydrogen in each of these groups may be independently replaced by a halogen, C₀-C₁₀ alkCO₂R², C₀-C₁₀ alkC(O)R², C₀-C₁₀ alkC(O)NR²R², C₀-C₁₀ alkOR², C₀-C₁₀ alkSR², alkNR²R², C₀-C₁₀ alkNR²C(O)R², C₀-C₁₀ alkNR²C(O)OR², C₀-C₁₀ alkyl, C₂-C₁₀ alkenyl, C₂-C₁₀ alkynyl, C₃-C₁₀ cycloalkyl, C₄-C₂₀ alkcycloalkyl, C₃-C₇ heterocycloalkyl, C₆-C₁₄ aryl, C₇-C₂₄ alkaryl, C₃-C₁₃ heteroaryl, C₄-C₂₃ alkheteroaryl, C₃-C₇ heterocycloalkyl and C₄-C₁₇ alkheterocycloalkyl; wherein R² is selected independently from the group consisting of hydrogen, C₁-C₁₀ alkyl, C₂-C₁₀ alkenyl, C₃-C₁₀ cycloalkyl, C₄-C₂₀ alkcycloalkyl, C₆-C₁₄ aryl, C₃-C₁₃ heteroaryl, C₇-C₂₄ alkaryl, C₃-C₇ heterocycloalkyl and C₄-C₁₇ alkheterocycloalkyl, wherein each hydrogen in each of these groups may be independently replaced a halogen.

It should be noted that the ring systems in the list above are all monovalent. Although A and C are monovalent ring systems, B is a divalent ring system, as can be clearly seen from structure (I). In this light, each monovalent ring system in the list above may be interpreted as any one of the corresponding divalent ring systems. For example, “phenyl” may be interpreted as phenyl (monovalent), or as 1,2-phenylene, 1,3-phenylene or 1,4-phenylene (divalent). A corresponding divalent ring system is defined as the monovalent ring system wherein any one hydrogen is substituted by a second valency. This comment applies mutatis mutandis to any list of ring systems from which A and/or B and/or C are to be selected in this application, unless explicitly stated otherwise.

It is clear that there are two orientations in which a divalent ring system B may be incorporated into a compound according to the invention. For example, if B is 2-fluoro-1,4-phenylene, two compounds (II) and (II′) may be referred to. Unless explicitly stated otherwise, whenever the general notation of B, as shown in the table below, or a general name such as “2-fluoro-1,4-phenylene” is used in this application, reference is made to both (II) and (II′). Whenever a specific notation of B is used, reference is made to one of the compounds (II) and (II′), as shown in the table below.

| General notation of B | Specific notation of B | Compound |
|---|---|---|
| 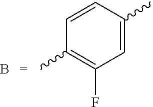 | 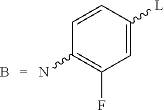 | 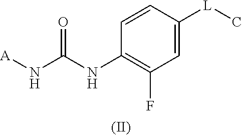 |
| | 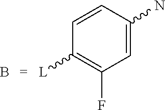 | 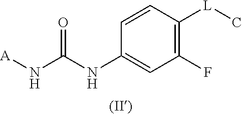 |

In the specific structures for B, it is clear that L and N are not comprised in B, but are merely present to indicate the orientation of B.

For all structures in this application representing a compound according to the invention, letters A and C refer to ring systems A and C if said letters correspond with monovalent groups, unless explicitly stated otherwise. For all structures in this application representing a compound according to the invention, letter B refers to ring system B if said letter corresponds with a divalent group, unless explicitly stated otherwise. Evidently, a letter C which corresponds with a tetravalent group should be interpreted as a carbon atom, whereas a letter B which corresponds with a trivalent group should be interpreted as a boron atom.

In a preferred embodiment is provided a compound especially for use in treating, ameliorating, delaying, curing and/or preventing a disease or condition associated with muscle cells and/or satellite cells, or a pharmaceutically acceptable salt thereof, wherein said compound is represented by structure (I),

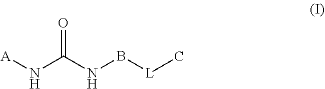

wherein B and C are ring systems comprising a five-membered or a six-membered aromatic or heteroaromatic ring, wherein said ring systems do not comprise more than 30 carbon atoms;
wherein A is a ring system selected from the group consisting of

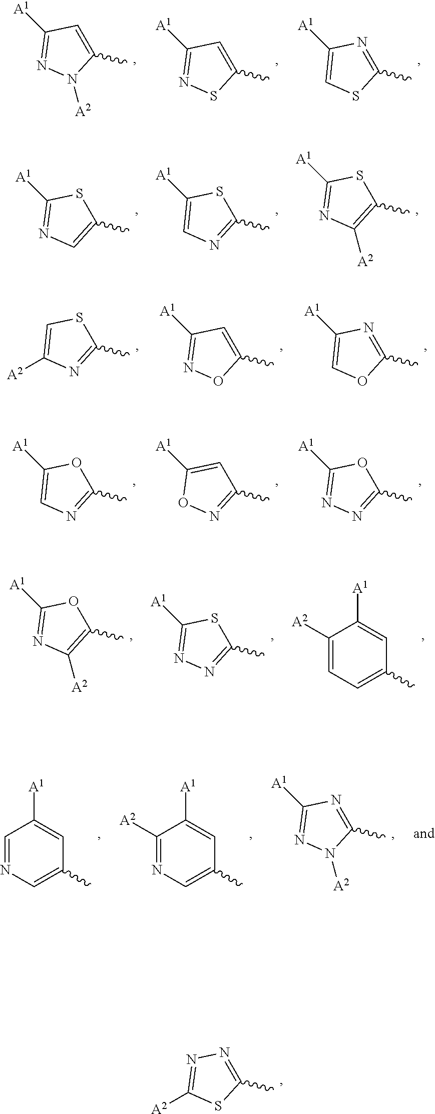

optionally from the group consisting of

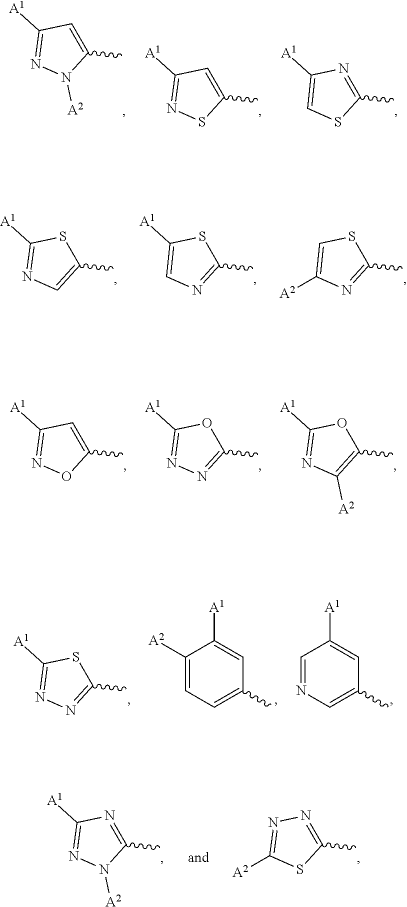

optionally from the group consisting of

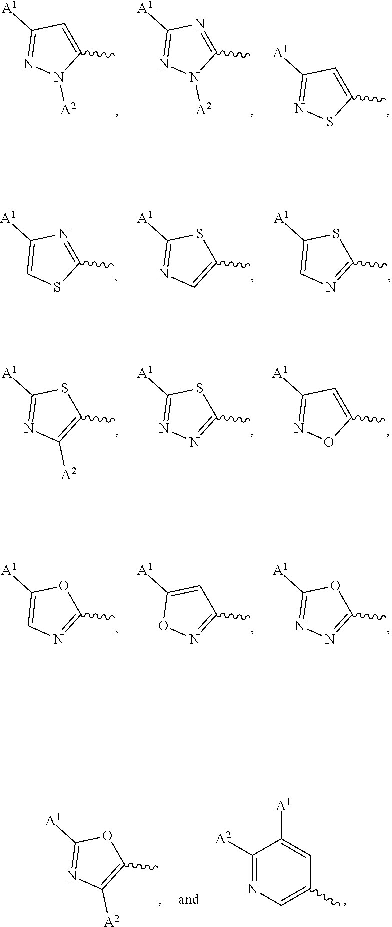

optionally wherein A is

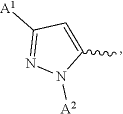

wherein A¹ is selected from the group consisting of C₁-C₄ alkyl, C₁-C₄ cycloalkyl,

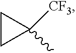

C₃-C₇ heterocycloalkyl,

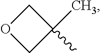

phenyl, benzyl and

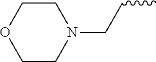

wherein A¹ is optionally fluorinated, preferably wherein A₁ is selected from the group consisting of methyl, —CF₃, isopropyl, cyclopropyl, isobutyl, sec-butyl, tert-butyl,

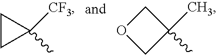

more preferably wherein A¹ is tert-butyl,
wherein A² is selected from the group consisting of H, C₁-C₄ alkyl, C₁-C₄ cycloalkyl, C₃-C₇ heterocycloalkyl, halogens, benzyl, phenyl, tolyl,

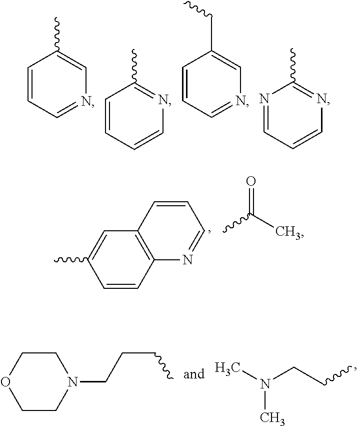

wherein A² is optionally substituted with 1, 2 or 3 substituents selected from the group consisting of C₁-C₄ alkyl, —Cl, —F, —CN, —C(O)—NR^{ABC}R^{ABC}, —OR^{ABC}, —SR^{ABC}, —NR^{ABC}R^{ABC}, —NR^{ABC}C(O)—R^{ABC}, —SO₂R^{ABC}, C₁-C₁₀ alkyl, C₃-C₁₀ cycloalkyl, C₃-C₇ heterocycloalkyl, C₃-C₁₃ heteroaryl and C₄-C₂₃ alkheteroaryl; wherein R^{ABC} is selected independently from the group consisting of C₁-C₁₀ alkyl, C₂-C₁₀ alkenyl, C₃-C₁₀ cycloalkyl, C₃-C₇ heterocycloalkyl, C₆-C₁₄ aryl, C₃-C₁₃ heteroaryl, C₇-C₂₄ alkaryl, wherein each hydrogen in each of these groups may be independently substituted by a halogen, C₃-C₇ heterocycloalkyl or NRR, with R being a C₁-C₃ alky, with A² being optionally fluorinated;
optionally A² is selected from the group consisting of H, C₁-C₄ alkyl, C₁-C₄ cycloalkyl, C₃-C₇ heterocycloalkyl, halogens, benzyl, phenyl, tolyl,

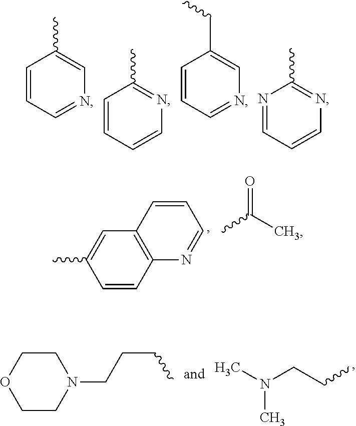

wherein A² is optionally substituted with a substituent selected from the group consisting of C₁-C₄ alkyl, —Cl, —F, —CN, —C(O)—NR^{ABC}R^{ABC}, —OR^{ABC}, —SR^{ABC}, —NR^{ABC}R^{ABC}, —NR^{ABC}C(O)—R^{ABC}, C₁-C₁₀ alkyl, C₃-C₁₀ cycloalkyl, C₃-C₇ heterocycloalkyl, C₃-C₁₃ heteroaryl and C₄-C₂₃ alkheteroaryl; wherein R^{ABC} is selected independently from the group consisting of C₁-C₁₀ alkyl, C₂-C₁₀ alkenyl, C₃-C₁₀ cycloalkyl, C₃-C₇ heterocycloalkyl, C₆-C₁₄ aryl, C₃-C₁₃ heteroaryl, C₇-C₂₄ alkaryl, wherein each hydrogen in each of these groups may be independently substituted by a halogen,
optionally A² is selected from the group consisting of —H, C₁-C₄ alkyl, C₃-C₆ cycloalkyl, C₃-C₇ heterocycloalkyl, halogen, benzyl, phenyl, tolyl,

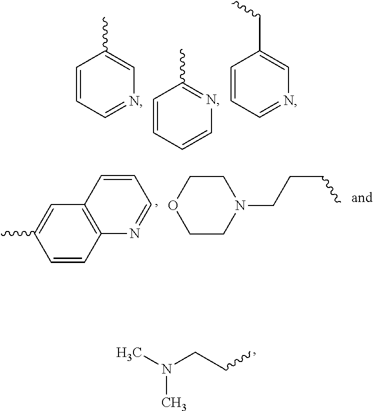

wherein A² is optionally fluorinated, and is optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,
optionally A² is selected from the group consisting of H, methyl, iso-propyl, benzyl, phenyl, chlorine,

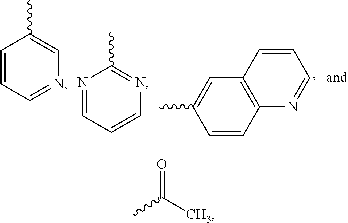

more specifically A² is phenyl;
wherein L is selected from the group consisting of —O—, —S—, —NR—, —(CH₂)ₘ—, —C(O)—, —CH(OH)—, —(CH₂)ₘO—, —(CH₂)ₘS—, —(CH₂)ₘNR—, —O(CH₂)ₘ—, —CHX—, —CX₂—, —S(CH₂)ₘ— and —NR(CH₂)ₘ—, m is 1, 2 or 3, X is a halogen,
R is selected from the group consisting of H, C₁-C₁₀ alkyl, C₂-C₁₀ alkenyl, C₃-C₁₀ cycloalkyl, C₆-C₁₄ aryl, C₃-C₁₃ heteroaryl and C₇-C₂₄ alkaryl, wherein R is optionally substituted with one or more halogen substituents, with L being optionally substituted,
preferably wherein L is O.
In a particular aspect, L is —O— and A is a ring system selected from the group consisting of

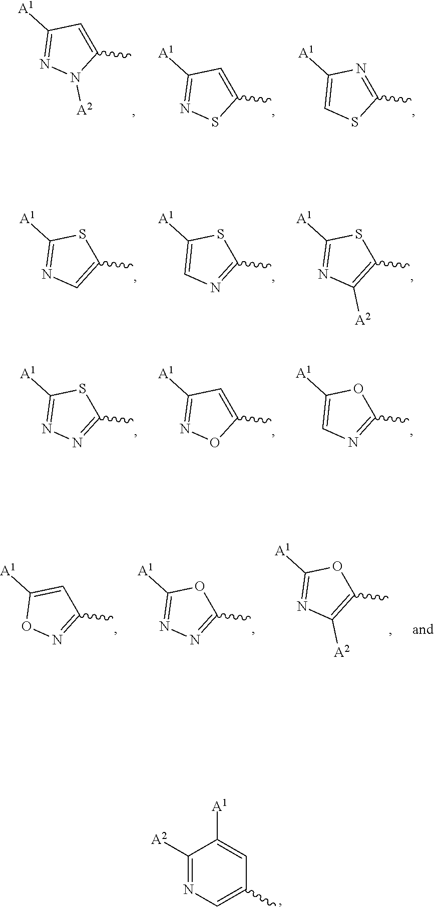

wherein A¹ is selected from the group consisting of C₁-C₄ alkyl, C₁-C₄ cycloalkyl,

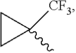

C₃-C₇ heterocycloalkyl,

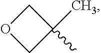

phenyl, benzyl and

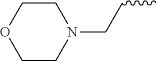

wherein A¹ is optionally fluorinated, and A² is selected from the group consisting of —H, C₁-C₄ alkyl, C₃-C₆ cycloalkyl, C₃-C₇ heterocycloalkyl, halogen, benzyl, phenyl, tolyl,

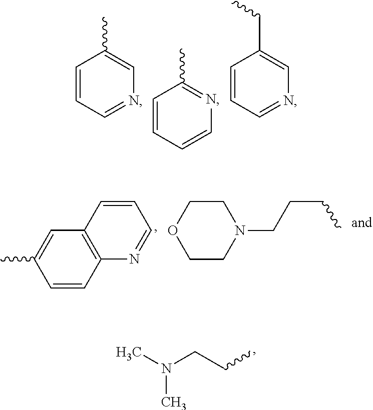

wherein A¹ and A² are optionally fluorinated, and A² is optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl.
More particularly, L is —O— and A is a ring selected from the group consisting of

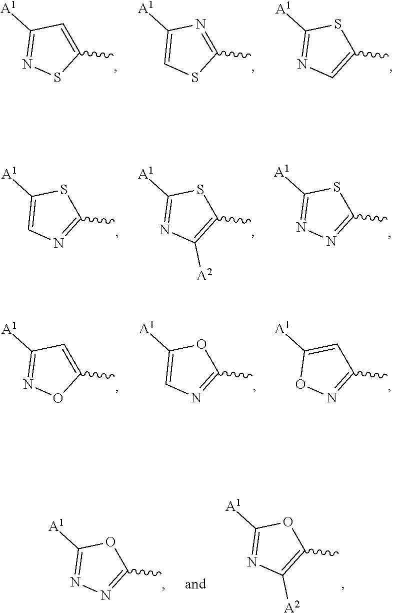

A¹ is selected from the group consisting of methyl, —CF₃, isopropyl, cyclopropyl, isobutyl, sec-butyl, tert-butyl,

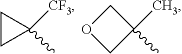

### and A² is —H.

More particularly, A¹ is tert-butyl.
More particularly, L is —O— and A is a ring selected from the group consisting of

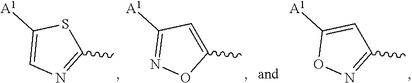

with A¹ being tert-butyl.

Hence, in a preferred embodiment is provided a compound according to the invention, wherein said compound is represented by structure (III),

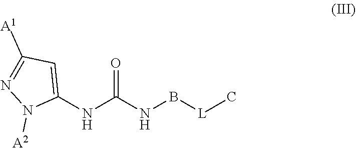

wherein A¹ is selected from the group consisting of C₁-C₄ alkyl, C₁-C₄ cycloalkyl,

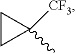

C₃-C₇ heterocycloalkyl,

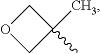

phenyl, benzyl and

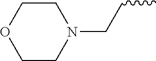

wherein A¹ is optionally fluorinated, and
wherein A² is selected from the group consisting of H, C₁-C₄ alkyl, C₁-C₄ cycloalkyl, benzyl, phenyl, tolyl,

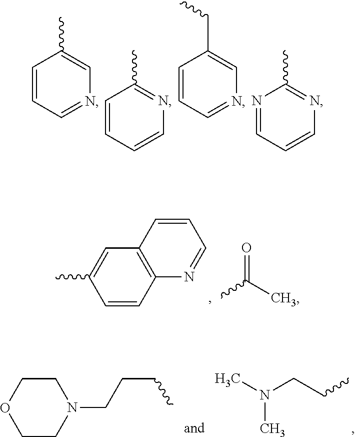

wherein A² is optionally substituted with a substituent selected from the group consisting of C₁-C₄ alkyl, —Cl, —F, —CN, C(O)NR^{ABC}R^{ABC}, —OR^{ABC}, —SR^{ABC}, —NR^{ABC}R^{ABC}, —NR^{ABC}C(O)—R^{ABC}, SO₂R^{ABC}, C₁-C₁₀ alkyl, C₃-C₁₀ cycloalkyl, C₃-C₇ heterocycloalkyl, C₃-C₁₃ heteroaryl and C₄-C₂₃ alkheteroaryl; wherein RAK is selected independently from the group consisting of C₁-C₁₀ alkyl, C₂-C₁₀ alkenyl, C₃-C₁₀ cycloalkyl, C₃-C₇ heterocycloalkyl, C₆-C₁₄ aryl, C₃-C₁₃ heteroaryl, C₇-C₂₄ alkaryl, wherein each hydrogen in each of these groups may be independently substituted by a halogen, C₃-C₇ heterocycloalkyl or NRR, with R being a C₁-C₃ alkyl, with A² being optionally fluorinated,
preferably wherein L is O.
In a particular aspect, A¹ is selected from the group consisting of methyl, —CF₃, isopropyl, cyclopropyl, isobutyl, sec-butyl, tert-butyl,

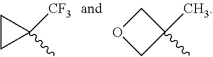

Preferably, A¹ is tert-butyl.
In a particular aspect, A² is selected from the group consisting of —H, C₁-C₄ alkyl, C₃-C₆ cycloalkyl, C₃-C₇ heterocycloalkyl, halogen, benzyl, phenyl, tolyl,

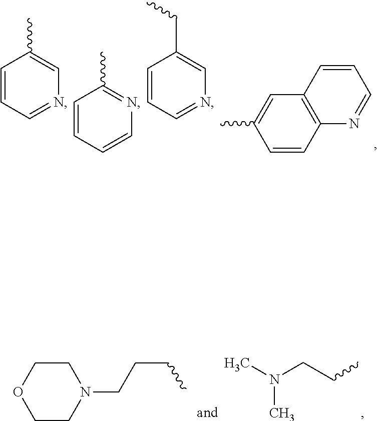

wherein A² is optionally fluorinated, and is optionally substituted with 1, 2 or 3 substituents selected from the group consisting of C₁-C₄ alkyl, —Cl, —F, —CN, —OR^{ABC}, and —SO₂R^{ABC}, wherein RAK is C₁-C₁₀ alkyl, and the substituent being optionally substituted by C₃-C₇ heterocycloalkyl or NRR, with R being a C₁-C₃ alkyl.
In a particular aspect, A² is selected from the group consisting of H, methyl, iso-propyl, benzyl, phenyl, chlorine,

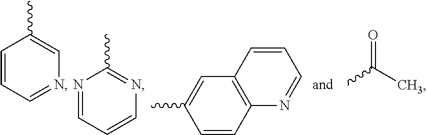

preferably a phenyl.
In a particular aspect, A¹ is selected from the group consisting of methyl, —CF₃, isopropyl, cyclopropyl, isobutyl, sec-butyl, tert-butyl,

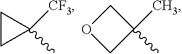

preferably tert-butyl;
and A² is selected from the group consisting of H, methyl, iso-propyl, benzyl, phenyl, chlorine,

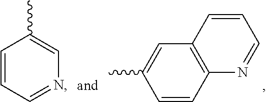

wherein A¹ and A² are optionally fluorinated, and A² is optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl.
In a more specific aspect, A¹ is tert-butyl, and A² is a phenyl or

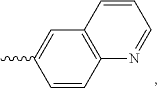

optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl.
In another more specific aspect, A¹ is tert-butyl, and A² is a phenyl optionally substituted with 1, 2 or 3 substituents selected from the group consisting of methyl, —Cl, —F, —CN, methoxy, —O—(CH₂)₂-piperidinyl, —O—(CH₂)₂-morpholinyl, —O—(CH₂)₃—N(CH₃)₂, and —CH₂-morpholinyl, or A² is

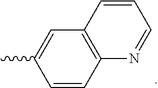

In another particular aspect, A¹ is selected from the group consisting of C₁-C₄ alkyl, C₁-C₄ cycloalkyl,

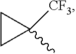

C₃-C₇ heterocycloalkyl,

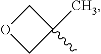

phenyl, benzyl and

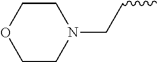

wherein A¹ is optionally fluorinated,
and A² is selected from the group consisting of a phenyl substituted with 1, 2 or 3 substituents selected from the group consisting of —Cl, —F, —CN, C₁-C₃ alkyloxy optionally substituted by C₃-C₇ heterocycloalkyl or NRR, with R being a C₁-C₃ alkyl, and C₁-C₄ alkyl substituted by C₃-C₇ heterocycloalkyl or NRR, with R being a C₁-C₃ alkyl; a phenyl or pyridinyl substituted by 2 or 3 C₁-C₃ alkyl; and a pyridinyl substituted with 1, 2 or 3 substituents selected from the group consisting of —Cl, —F, —CN, C₁-C₁₀ alkyloxy substituted by C₃-C₇ heterocycloalkyl or NRR, with R being a C₁-C₃ alkyl, and C₁-C₄ alkyl substituted by C₃-C₇ heterocycloalkyl or NRR, with R being a C₁-C₃ alkyl.
Preferably, A¹ is selected from the group consisting of methyl, —CF₃, isopropyl, cyclopropyl, isobutyl, sec-butyl, tert-butyl,

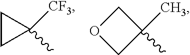

preferably tert-butyl,
and A² is selected from the group consisting of a phenyl substituted with 1, 2 or 3 substituents selected from the group consisting of methoxy, —Cl, —F, —CN, —O—(CH₂)₂-piperidinyl, —O—(CH₂)₂-morpholinyl, —O—(CH₂)₃—N(CH₃)₂, and —CH₂-morpholinyl; a phenyl or pyridinyl substituted with 2 or 3 methyl, and a pyridinyl substituted with 1, 2 or 3 substituents selected from the group consisting of methyl, —Cl, —F, —CN, —O—(CH₂)₂-piperidinyl, —O—(CH₂)₂-morpholinyl, —O—(CH₂)₃—N(CH₃)₂, and —CH₂-morpholinyl.
In a very specific aspect, A¹ is tert-butyl,
and A² is selected from the group consisting of a phenyl substituted with 1, 2 or 3 substituents selected from the group consisting of —Cl, —F, —CN, —O—(CH₂)₂-piperidinyl, —O—(CH₂)₃—N(CH₃)₂, and —CH₂-morpholinyl;
a pyridinyl substituted with a methyl; and a phenyl substituted by two methyl.

In a more preferred embodiment is provided a compound according to the invention, wherein said compound is represented by structure (IV),

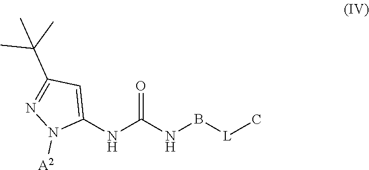

wherein A² is selected from the group consisting of H, C₁-C₄ alkyl, C₁-C₄ cycloalkyl, benzyl, phenyl, tolyl,

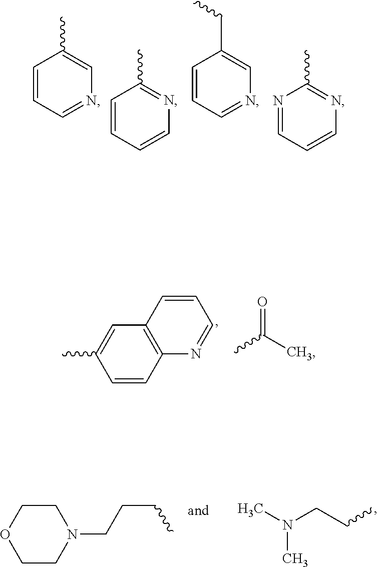

wherein A² is optionally substituted with a substituent selected from the group consisting of C₁-C₄ alkyl, —Cl, —F, —CN, C(O)NR^{ABC}R^{ABC}, —OR^{ABC}, —SR^{ABC}, —NR^{ABC}R^{ABC}, —NR^{ABC}C(O)—R^{ABC}, SO₂R^{ABC}, C₁-C₁₀ alkyl, C₃-C₁₀ cycloalkyl, C₃-C₇ heterocycloalkyl, C₃-C₁₃ heteroaryl and C₄-C₂₃ alkheteroaryl; wherein R^{ABC} is selected independently from the group consisting of C₁-C₁₀ alkyl, C₂-C₁₀ alkenyl, C₃-C₁₀ cycloalkyl, C₃-C₇ heterocycloalkyl, C₆-C₁₄ aryl, C₃-C₁₃ heteroaryl, C₇-C₂₄ alkaryl, wherein each hydrogen in each of these groups may be independently substituted by a halogen, C₃-C₇ heterocycloalkyl or NRR, with R being a C₁-C₃ alkyl, with A² being optionally fluorinated,
preferably wherein L is O.
Preferably, A² is selected from the group consisting of H, methyl, iso-propyl, benzyl, phenyl, chlorine,

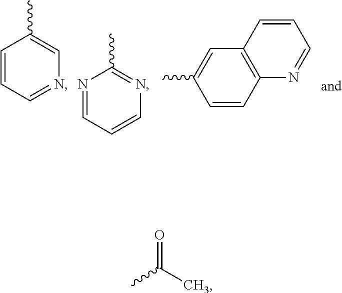

more preferably A² is phenyl,
In a particular aspect, A² is selected from the group consisting of H, methyl, iso-propyl, benzyl, phenyl, chlorine,

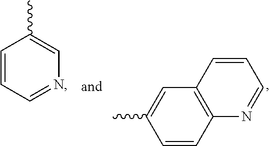

wherein A¹ and A² are optionally fluorinated, and A² is optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl.
In more specific aspect, A² is a phenyl or

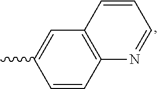

optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl.
In another more specific aspect, A² is a phenyl optionally substituted with 1, 2 or 3 substituents selected from the group consisting of methyl, —Cl, —F, —CN, methoxy, —O—(CH₂)₂-piperidinyl, —O—(CH₂)₂-morpholinyl, —O—(CH₂)₃—N(CH₃)₂, and —CH₂-morpholinyl, or A² is

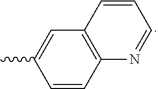

In another particular aspect, A² is selected from the group consisting of a phenyl substituted with 1, 2 or 3 substituents selected from the group consisting of —Cl, —F, —CN, C₁-C₃ alkyloxy optionally substituted by C₃-C₇ heterocycloalkyl or NRR, with R being a C₁-C₃ alkyl, and C₁-C₄ alkyl substituted by C₃-C₇ heterocycloalkyl or NRR, with R being a C₁-C₃ alkyl; a phenyl or pyridinyl substituted by 2 or 3 C₁-C₃ alkyl; and a pyridinyl substituted with 1, 2 or 3 substituents selected from the group consisting of —Cl, —F, —CN, C₁-C₁₀ alkyloxy substituted by C₃-C₇ heterocycloalkyl or NRR, with R being a C₁-C₃ alkyl, and C₁-C₄ alkyl substituted by C₃-C₇ heterocycloalkyl or NRR, with R being a C₁-C₃ alkyl.
In a more specific aspect, A² is selected from the group consisting of a phenyl substituted with 1, 2 or 3 substituents selected from the group consisting of methoxy, —Cl, —F, —CN, —O—(CH₂)₂-piperidinyl, —O—(CH₂)₂-morpholinyl, —O—(CH₂)₃—N(CH₃)₂, and —CH₂-morpholinyl; a phenyl or pyridinyl substituted with 2 or 3 methyl, and a pyridinyl substituted with 1, 2 or 3 substituents selected from the group consisting of methyl, —Cl, —F, —CN, —O—(CH₂)₂-piperidinyl, —O—(CH₂)₂-morpholinyl, —O—(CH₂)₃—N(CH₃)₂, and —CH₂-morpholinyl.
In a very specific aspect, A² is selected from the group consisting of a phenyl substituted with 1, 2 or 3 substituents selected from the group consisting of —Cl, —F, —CN, —O—(CH₂)₂-piperidinyl, —O—(CH₂)₃—N(CH₃)₂, and —CH₂-morpholinyl; a pyridinyl substituted with a methyl; and a phenyl substituted by two methyl.

In another more preferred embodiment is provided a compound according to the invention, wherein said compound is represented by structure (V),

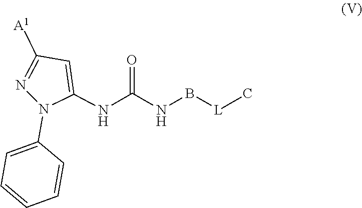

wherein A¹ is selected from the group consisting of C₁-C₄ alkyl, C₁-C₄ cycloalkyl,

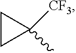

C₃-C₇ heterocycloalkyl,

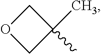

phenyl, benzyl and

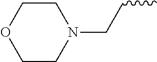

wherein A¹ is optionally fluorinated, preferably wherein A¹ is selected from the group consisting of methyl, —CF₃, isopropyl, cyclopropyl, isobutyl, sec-butyl, tert-butyl,

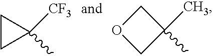

more preferably wherein A¹ is tert-butyl, preferably wherein L is O.

In a most preferred embodiment is provided a compound according to the invention, wherein said compound is represented by structure (VI), preferably wherein L is O,

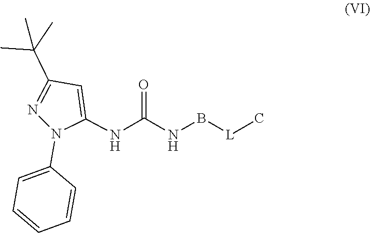

In a preferred embodiment is provided a compound especially for use in treating, ameliorating, delaying, curing and/or preventing a disease or condition associated with muscle cells and/or satellite cells, or a pharmaceutically acceptable salt thereof, wherein said compound is represented by structure (I),

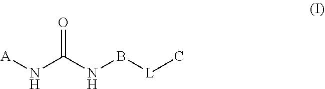

wherein A and C are ring systems comprising a five-membered or a six-membered aromatic or heteroaromatic ring, wherein said ring systems do not comprise more than 30 carbon atoms;
wherein B is a ring system selected from the group consisting of

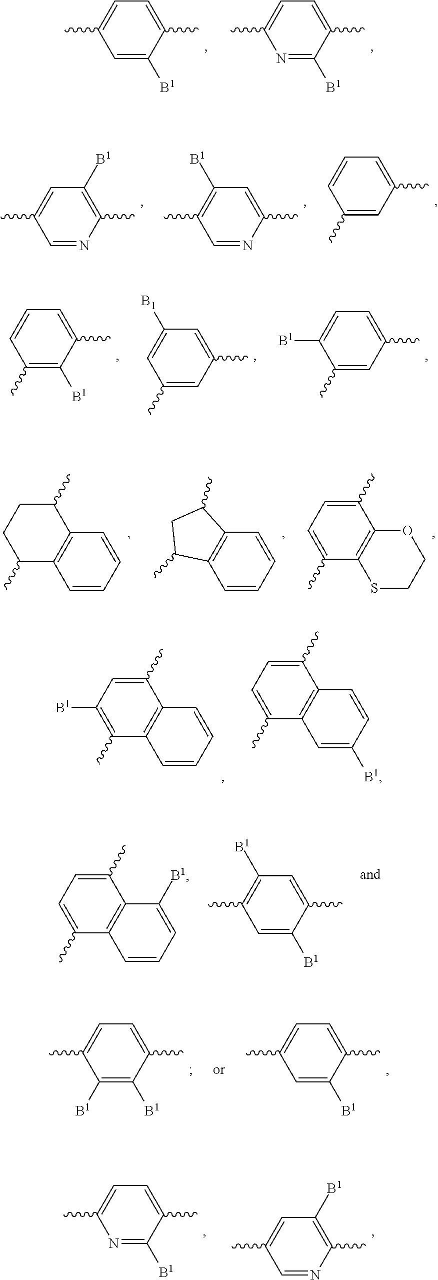

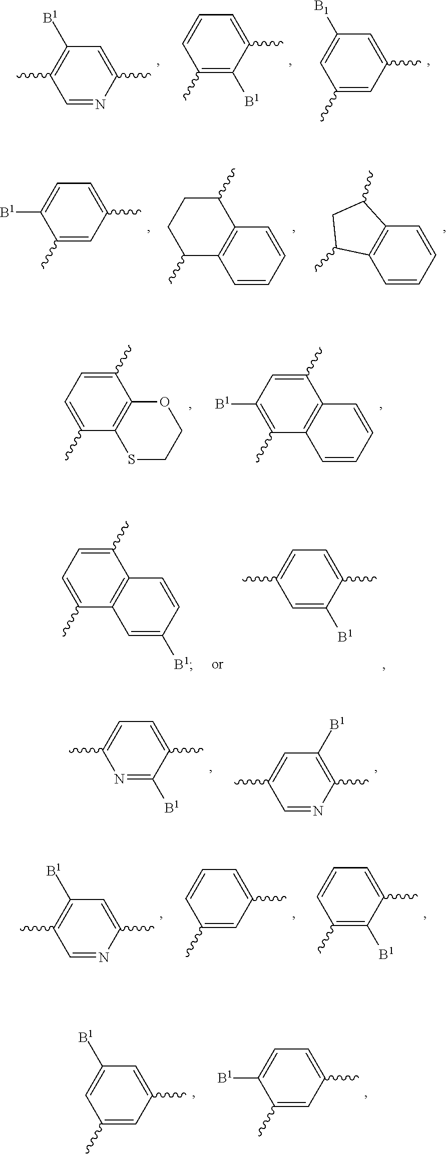

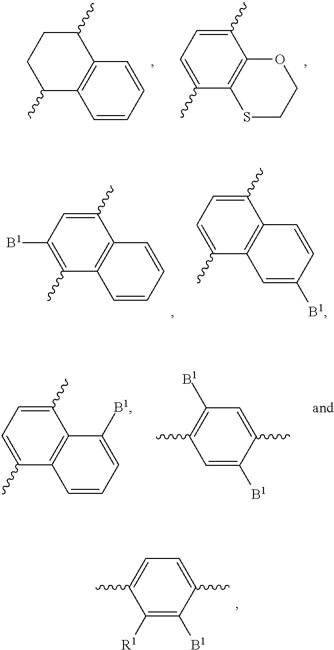

wherein B¹ is selected from the group consisting of —H, —F, —Cl, —SCH₃, —SCH₂CH₃, isopropyl, —CF₃, —OCH₃, and —OCF₃ and wherein two B¹ can be linked to form a fused bicyclic ring system containing 0 to 3 heteroatoms; optionally from the group consisting of —H, —F, —SCH₃, —SCH₂CH₃, isopropyl, —CF₃; optionally B¹ is not H when B is a phenyl;
wherein L is selected from the group consisting of —O—, —S—, —NR—, —(CH₂)ₘ, —C(O)—, —CH(OH)—, —(CH₂)ₘO—, —(CH₂)ₘS—, —(CH₂)ₘNR—, —O(CH₂)ₘ—, —CHX—, —CX₂—, —S(CH₂)ₘ— and —NR(CH₂)ₘ—, m is 1, 2 or 3, X is a halogen,
R is selected from the group consisting of H, C₁-C₁₀ alkyl, C₂-C₁₀ alkenyl, C₃-C₁₀ cycloalkyl, C₆-C₁₄ aryl, C₃-C₁₃ heteroaryl and C₇-C₂₄ alkaryl, wherein R is optionally substituted with one or more halogen substituents, L being optionally substituted, preferably L is —O—.
In an aspect, B is a ring system selected from the group consisting of

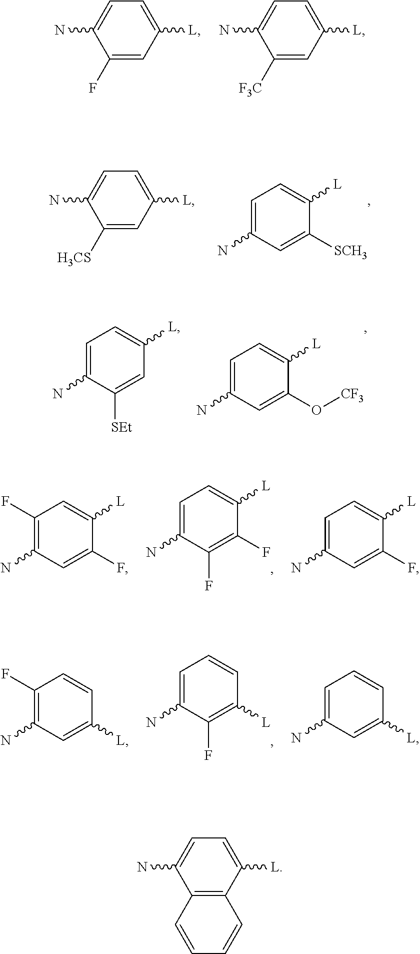

In a specific aspect, B is a ring system selected from the group consisting of

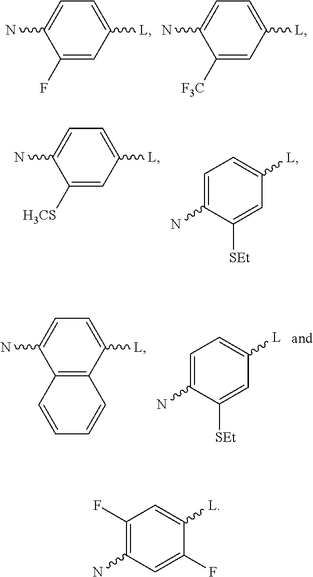

In a very specific aspect, B is a ring system selected from the group consisting of

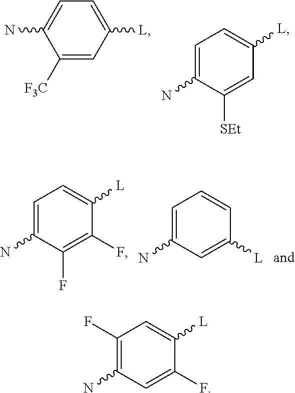

In a specific aspect, B is a ring system selected from the group consisting of

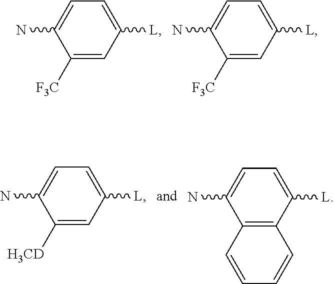

Hence, in a preferred embodiment is provided a compound according to the invention, wherein said compound is represented by structures (VII-a)-(VII-m), preferably wherein L is O,

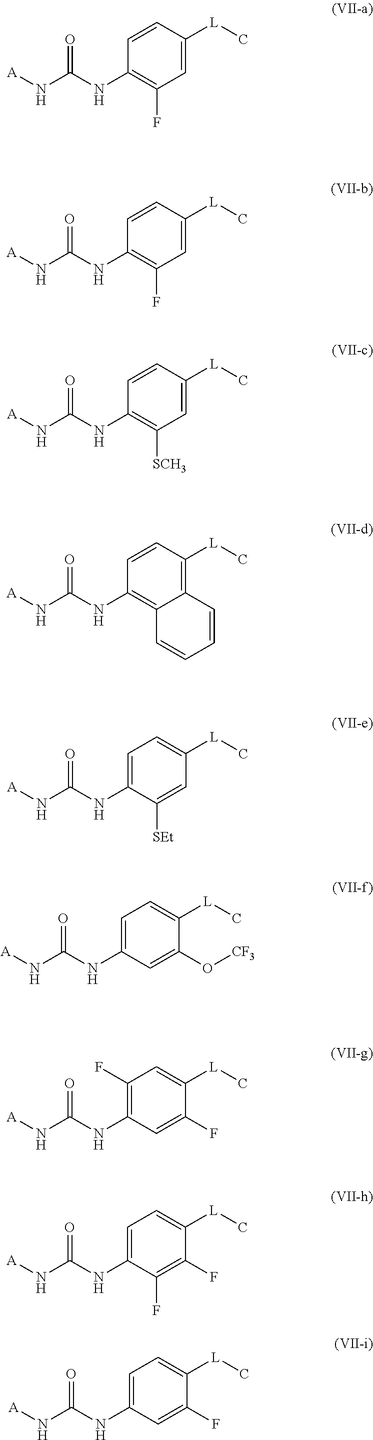

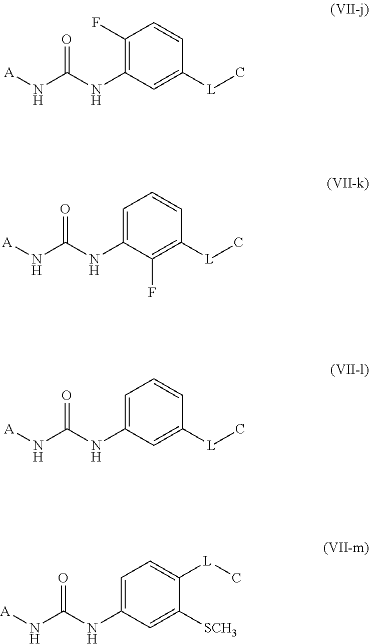

In a specific aspect, the compound may be represented by the structures (VII-a), (VII-b), (VII-c), (VII-d), (VII-e), (VII-g), and (VII-h).
In another specific aspect, the compound may be represented by the structures (VII-b), (VII-e), (VII-g), (VII-h) and (VII-I).
In another specific aspect, the compound may be represented by the structures (VII-a)-(VII-d).

In a preferred embodiment is provided a compound especially for use in treating, ameliorating, delaying, curing and/or preventing a disease or condition associated with muscle cells and/or satellite cells, or a pharmaceutically acceptable salt thereof, wherein said compound is represented by structure (I),

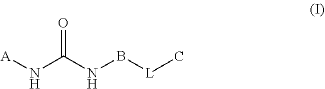

wherein A, B and C are ring systems comprising a five-membered or a six-membered aromatic or heteroaromatic ring, wherein said ring systems do not comprise more than 30 carbon atoms;
wherein L is selected from the group consisting of —O—, —S—, —CH₂— and —C(O)—, preferably wherein L is selected from the group consisting of —O— and —S—, more preferably wherein L is —O—.

Hence, in a more preferred embodiment is provided a compound according to the invention, wherein said compound is represented by structure (VIII),

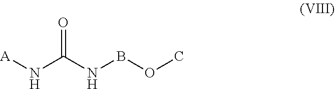

In the context of the compounds of formula (VIII), A, and B can be selected in any particular aspect described above and any combination of A and B.

In a preferred embodiment is provided a compound especially for use in treating, ameliorating, delaying, curing and/or preventing a disease or condition associated with muscle cells and/or satellite cells, or a pharmaceutically acceptable salt thereof, wherein said compound is represented by structure (I),

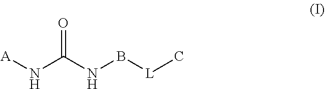

wherein A, B and C are ring systems comprising a five-membered or a six-membered aromatic or heteroaromatic ring, wherein said ring systems do not comprise more than 30 carbon atoms;
wherein ring system C is a monocyclic or a bicyclic ring system,
wherein said monocyclic ring system comprises a pyridine ring or a pyrimidine ring, wherein said pyridine or pyrimidine ring is optionally substituted,
preferably wherein said monocyclic system is selected from the group consisting of

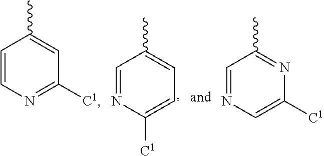

wherein C¹ is selected from the group consisting of wherein C¹ is selected from the group consisting of —H, —NH₂, —NR′, —C(O)—NH—R¹, —NH—C(O)—R¹, —NH—S(O)₂—R¹, —NH—C(O)—OR¹, —C(O)—R¹, —R¹, —OR′, —SO₂R¹, with R¹ being selected from the group consisting of C₁-C₁₀ alkyl, C₂-C₁₀ alkenyl, C₃-C₁₀ cycloalkyl, C₄-C₂₀ alkcycloalkyl, C₆-C₁₄ aryl, C₃-C₁₃ heteroaryl, C₇-C₂₄ alkaryl, C₃-C₇ heterocycloalkyl and C₄-C₁₇ alkheterocycloalkyl, wherein each hydrogen in each of these groups may be independently replaced by a group selected from halogen, hydroxy, —OR′, —COR′, —COOR′, and —NR′R″, each hydrogen in each of R′ and R″ may be independently replaced by —COR′″ or COOR′″, wherein R′, R″ and R′″ are independently a C₁-C₆ alkyl;
wherein said bicyclic ring system comprises two nitrogen atoms, and two six-membered rings or a six-membered ring and a five-membered ring,
preferably wherein said bicyclic system is selected from the group consisting of

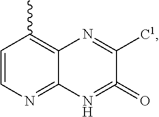

in particular

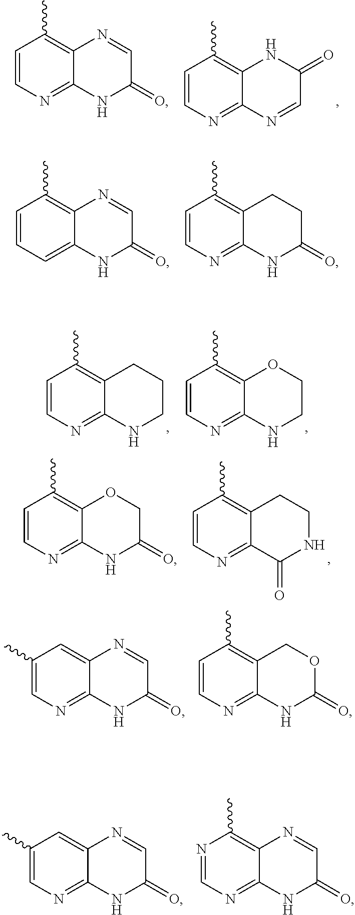

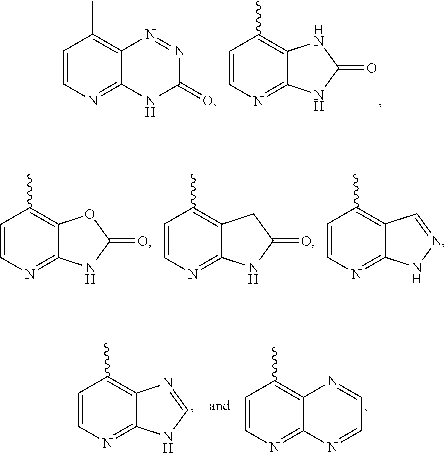

wherein C¹ is selected from the group consisting of —H, —CH₃, —OCH₃, —CN or

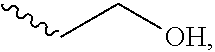

more preferably wherein C¹ is —H,
wherein L is selected from the group consisting of —O—, —S—, —NR—, —(CH₂)ₘ—, —C(O)—, —CH(OH)—, —(CH₂)ₘO—, —(CH₂)ₘS—, —(CH₂)ₘNR—, —O(CH₂)ₘ—, —CHX—, —CX₂—, —S(CH₂)ₘ— and —NR(CH₂)ₘ—, wherein m is 1, 2 or 3, wherein X is a halogen, wherein R is selected from the group consisting of H, C₁-C₁₀ alkyl, C₂-C₁₀ alkenyl, C₃-C₁₀ cycloalkyl, C₆-C₁₄ aryl, C₃-C₁₃ heteroaryl and C₇-C₂₄ alkaryl, wherein R is optionally substituted with one or more halogen substituents, wherein L is optionally substituted, preferably L is O.

Optionally, the compound is represented by structure (I),

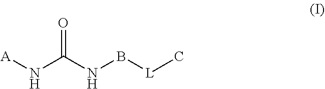

wherein said monocyclic ring system comprises a pyridine ring, wherein said pyridine ring is optionally substituted,
preferably wherein said monocyclic system is selected from the group consisting of

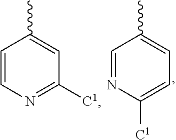

more preferably wherein said monocyclic system is

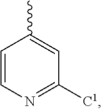

wherein C¹ is selected from the group consisting of —H, —NH₂,

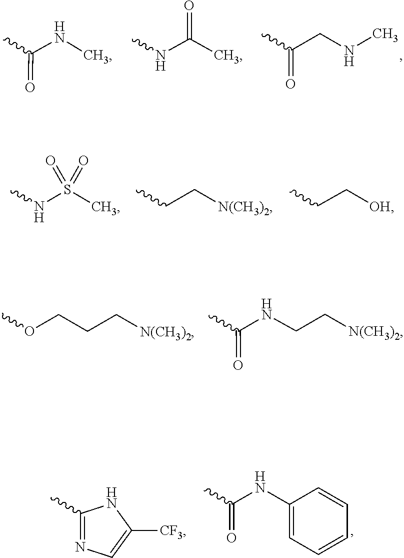

more preferably wherein C¹ is selected from the group consisting of

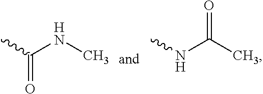

most preferably wherein C¹ is

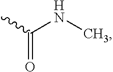

wherein said bicyclic ring system comprises two nitrogen atoms, and two six-membered rings or a six-membered ring and a five-membered ring,
preferably wherein said bicyclic system is selected from the group consisting of

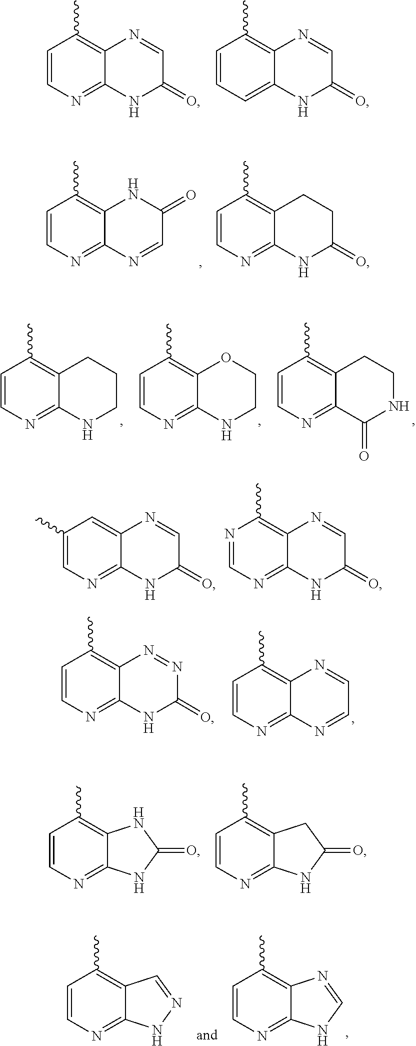

wherein said bicyclic system is optionally substituted with —OCH₃ or —CN,
optionally wherein said bicyclic system is

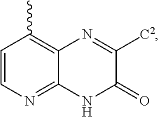

wherein C² is selected from the group consisting of —H, —OCH₃ and —CN, most preferably wherein C² is —H,
wherein L is selected from the group consisting of —O—, —S—, —NR—, —(CH₂)ₘ—, —C(O)—, —CH(OH)—, —(CH₂)ₘO—, —(CH₂)ₘS—, —(CH₂)ₘNR—, —O(CH₂)ₘ—, —CHX—, —CX₂—, —S(CH₂)ₘ— and —NR(CH₂)ₘ—, wherein m is 1, 2 or 3, wherein X is a halogen, wherein R is selected from the group consisting of H, C₁-C₁₀ alkyl, C₂-C₁₀ alkenyl, C₃-C₁₀ cycloalkyl, C₆-C₁₄ aryl, C₃-C₁₃ heteroaryl and C₇-C₂₄ alkaryl, wherein R is optionally substituted with one or more halogen substituents, wherein L is optionally substituted, preferably L is O.
A, and B can be selected in any particular aspect described above and any combination of A and B.

In a particular aspect, is provided a compound represented by structure (I),

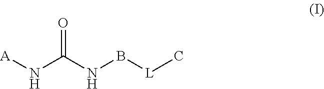

wherein C is selected from the group consisting of

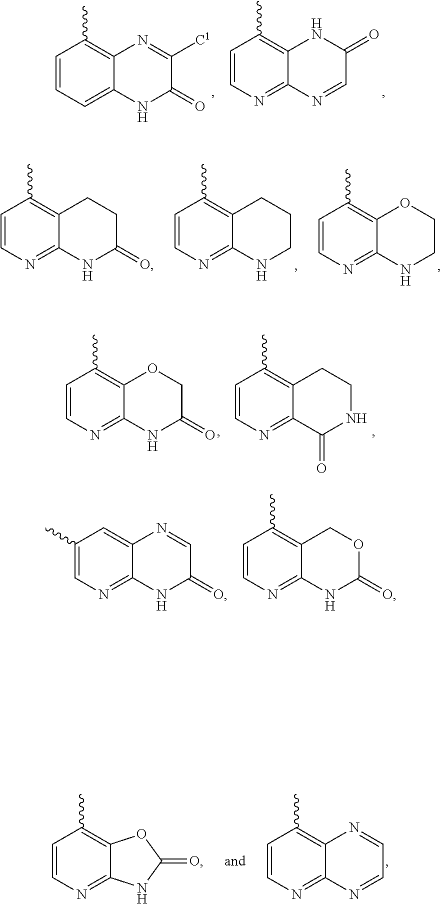

wherein C¹ is selected from the group consisting of —H, —CH₃, —OCH₃, —CN or

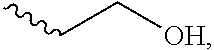

or C is selected from the group consisting of

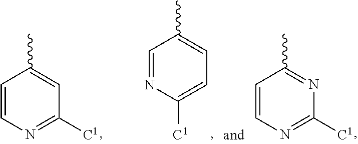

wherein C¹ is selected from the group consisting of wherein C¹ is selected from the group consisting of —H, —NH₂, —NR¹, —C(O)—NH—R¹, —NH—C(O)—R¹, —NH—S(O)₂—R¹, —NH—C(O)—OR¹, —C(O)—R¹, —R¹, —OR¹, —SO₂R¹, with R¹ being selected from the group consisting of C₁-C₁₀ alkyl, C₂-C₁₀ alkenyl, C₃-C₁₀ cycloalkyl, C₄-C₂₀ alkcycloalkyl, C₆-C₁₄ aryl, C₃-C₁₃ heteroaryl, C₇-C₂₄ alkaryl, C₃-C₇ heterocycloalkyl and C₄-C₁₇ alkheterocycloalkyl, wherein each hydrogen in each of these groups may be independently replaced by a group selected from halogen, hydroxy, —OR′, —COR′, —COOR′, and —NR′R″, each hydrogen in each of R′ and R″ may be independently replaced by —COR′″ or COOR′″, wherein R′, R″ and R′″ are independently a C₁-C₆ alkyl.
A and B can be selected in any particular aspect described above and any combination of A and B.
In a specific aspect, C is selected from the group consisting of

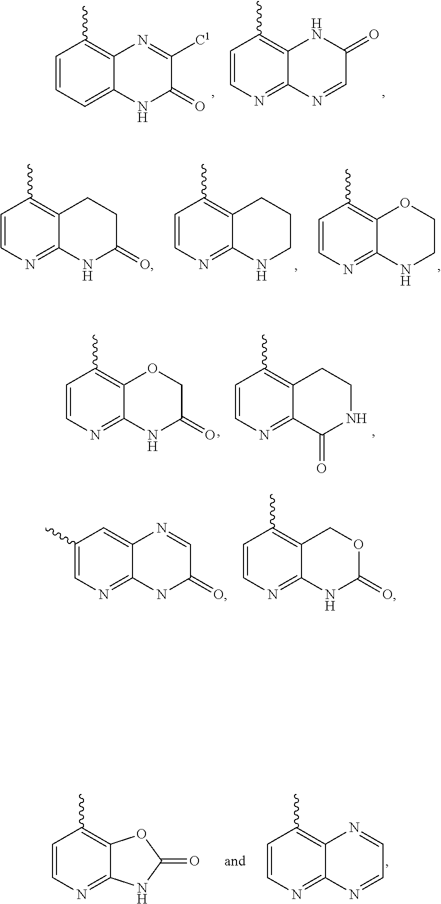

wherein C¹ is selected from the group consisting of —H, —CH₃, —OCH₃, —CN or

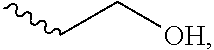

or
C is selected from the group consisting of

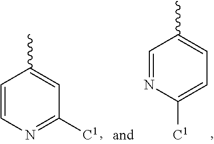

with C¹ being selected from the group consisting of —H, —NH₂,

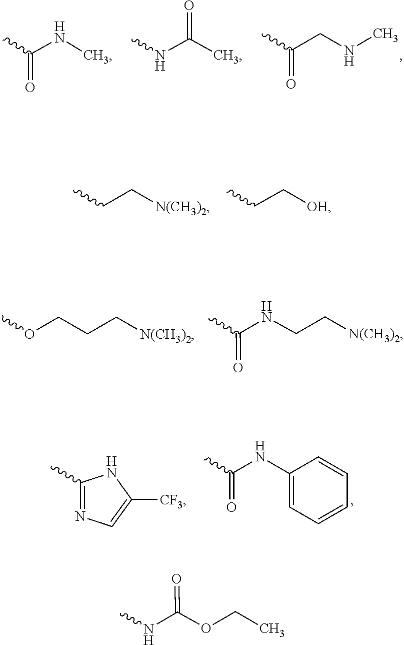

—NH—C(O)—OtBu, —NH—C(O)—N(CH₃)—C(O)—OtBu, —NH—C(O)—CH₂—N(CH₃)—C(O)—OtBu, —NH—C(O)—CH₂—NHCH₃, —NH—C(O)—Obenzyl, —NH—C(O)—CH₂—OH, —NH—(CH₂)₂—N(CH₃)₂, and —NH—S(O)₂—CH₃; preferably from the group consisting of —H,

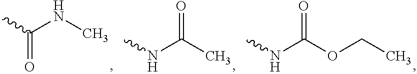

—NH—C(O)—OtBu, —NH—C(O)—N(CH₃)—C(O)—OtBu, —NH—C(O)—CH₂—N(CH₃)—C(O)—OtBu, —NH—C(O)—CH₂—NHCH₃, —NH—C(O)—Obenzyl, and —NH—C(O)—CH₂—OH.
A, and B can be selected in any particular aspect described above and any combination of A and B.
In another specific aspect, C is selected in the group consisting of

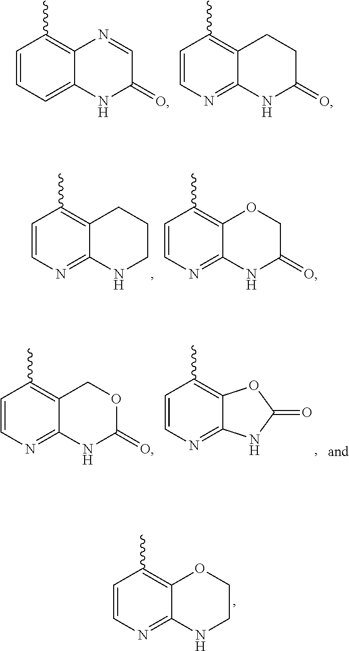

or C is selected from the group consisting of

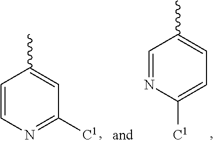

with C¹ being selected from the group consisting of

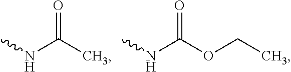

—NH—C(O)—OtBu, —NH—C(O)—N(CH₃)—CO—OtBu and —NH—C(O)—CH₂—N(CH₃)—C(O)—OtBu.
A, and B can be selected in any particular aspect described above and any combination of A and B.

In another aspect, s provided a compound represented by structure (I),

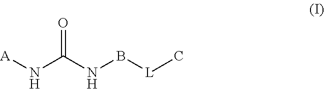

wherein C is selected from the group consisting of

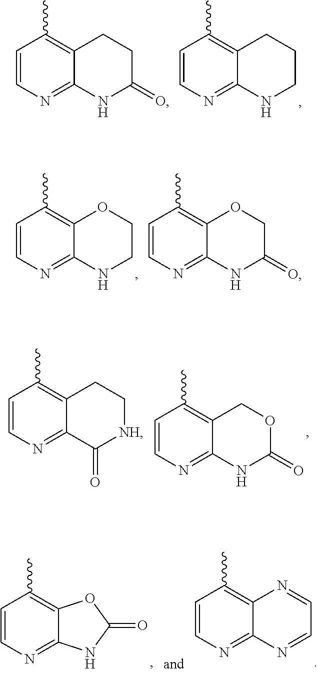

A, and B can be selected in any particular aspect described above and any combination of A and B.
In a specific aspect, C is selected from the group consisting of

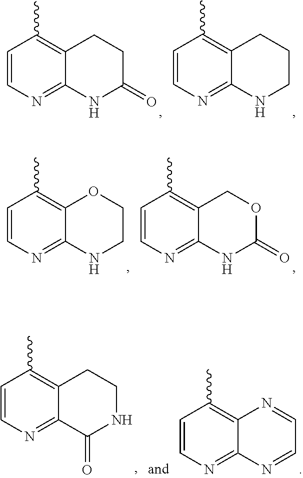

A, and B can be selected in any particular aspect described above and any combination of A and B.
In another specific aspect, C is selected from the group consisting of

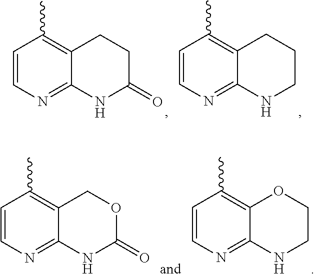

A, and B can be selected in any particular aspect described above and any combination of A and B.
In a specific aspect; C is selected from the group consisting of

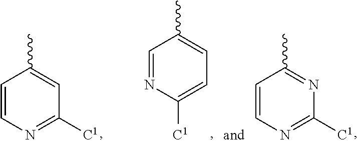

wherein C¹ is selected from the group consisting of —NH—C(O)R¹ or —NH—C(O)—OR¹ with R¹ being selected from the group consisting of C₁-C₁₀ alkyl, C₆-C₁₄ aryl, C₃-C₁₃ heteroaryl, and C₇-C₂₄ alkaryl, wherein each hydrogen in each of these groups may be independently replaced by a group selected from halogen, hydroxy, —OR′, and —NR′R″, each hydrogen in each of R′ and R″ may be independently replaced by —COR′″ or COOR′″, wherein R′, R″ and R′″ are independently a C₁-C₆ alkyl. Optionally, C¹ is selected from the group consisting of —NH—C(O)R′ or —NH—C(O)—OR¹ with R¹ being selected from the group consisting of C₁-C₁₀ alkyl, C₆-C₁₄ aryl, C₃-C₁₃ heteroaryl, and C₇-C₂₄ alkaryl, wherein each hydrogen in each of these groups may be independently replaced by a group selected from halogen, hydroxy, and —NR′R″, each hydrogen in each of R′ and R″ may be independently replaced by —COR′″ or COOR′″, wherein R′, R″ and R″ are independently a C₁-C₆ alkyl.
A, and B can be selected in any particular aspect described above and any combination of A and B.
In a specific aspect, C is selected from the group consisting of

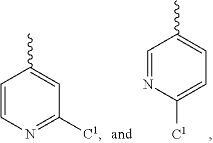

with C¹ being selected from the group consisting of

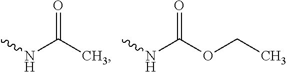

—NH—C(O)—OtBu, —NH—C(O)—N(CH₃)—C(O)—OtBu, —NH—C(O)—CH₂—N(CH₃)—C(O)—OtBu, —NH—C(O)—CH₂—NHCH₃, —NH—C(O)—Obenzyl, —NH—C(O)—CH₂—OH, preferably

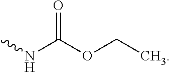

A, and B can be selected in any particular aspect described above and any combination of A and B.
In a particular aspect, the ring C is

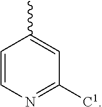

Hence, in a more preferred embodiment is provided a compound according to the invention, wherein said compound is represented by structure (IX) or structure (X), preferably wherein L is O,

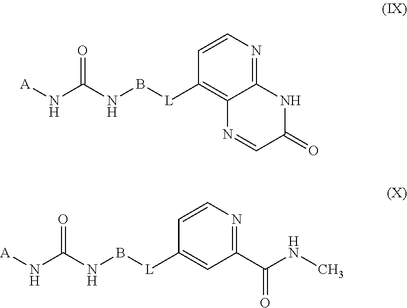

In a particular aspect, the compound is represented by structure (IX) or structure (X), and A is selected from the group consisting of

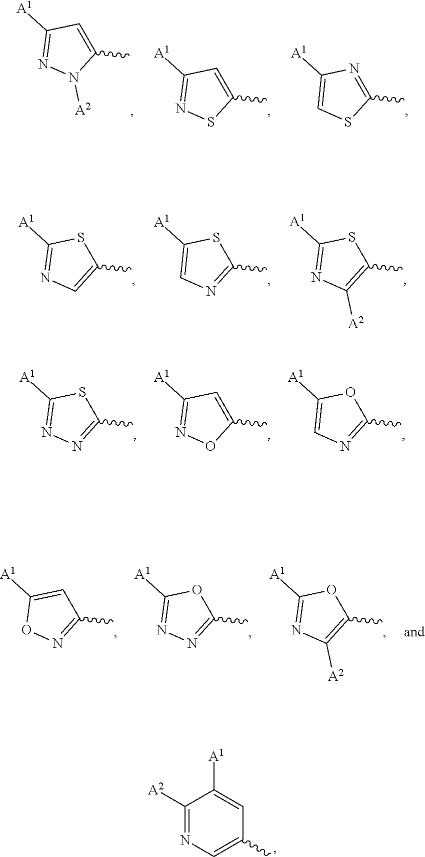

wherein A¹ is selected from the group consisting of C₁-C₄ alkyl, C₁-C₄ cycloalkyl,

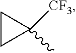

C₃-C₇ heterocycloalkyl,

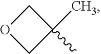

phenyl, benzyl and

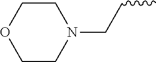

wherein A¹ is optionally fluorinated,
and A² is selected from the group consisting of —H, C₁-C₄ alkyl, C₃-C₆ cycloalkyl, C₃-C₇ heterocycloalkyl, halogen, benzyl, phenyl, tolyl,

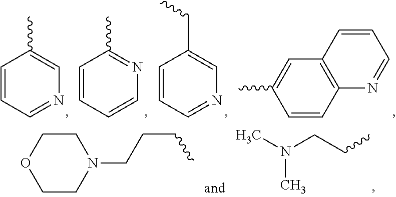

wherein A¹ and A² are optionally fluorinated, and A² is optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl.
B can be any ring as disclosed above.
In another particular aspect, the compound is represented by structure (IX) or structure (X), and B is selected in the group consisting of

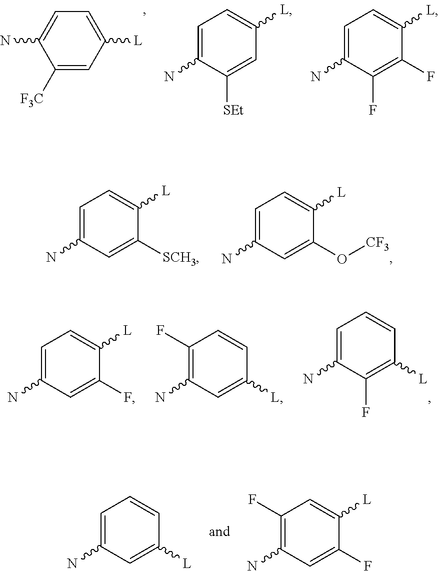

preferably from the group consisting of

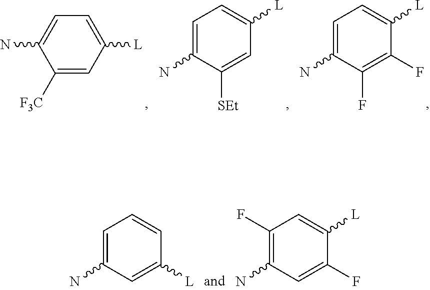

A can be any ring as disclosed above.
In another particular aspect, the compound is represented by structure (IX) or structure (X), A is selected from the group consisting of

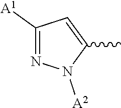

wherein A¹ is selected from the group consisting of C₁-C₄ alkyl, C₁-C₄ cycloalkyl,

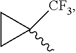

C₃-C₇ heterocycloalkyl,

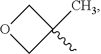

phenyl, benzyl and

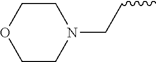

wherein A¹ is optionally fluorinated,
and A² is selected from the group consisting of a phenyl substituted with 1, 2 or 3 substituents selected from the group consisting of —Cl, —F, —CN, C₁-C₃ alkyloxy optionally substituted by C₃-C₇ heterocycloalkyl or NRR, with R being a C₁-C₃ alkyl, and C₁-C₄ alkyl substituted by C₃-C₇ heterocycloalkyl or NRR, with R being a C₁-C₃ alkyl; a phenyl or pyridinyl substituted by 2 or 3 C₁-C₃ alkyls; and a pyridinyl substituted with 1, 2 or 3 substituents selected from the group consisting of —Cl, —F, —CN, alkyloxy substituted by C₃-C₇ heterocycloalkyl or NRR, with R being a C₁-C₃ alkyl, and C₁-C₄ alkyl substituted by C₃-C₇ heterocycloalkyl or NRR, with R being a C₁-C₃ alkyl.
More particularly, A can be

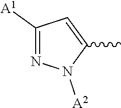

A¹ is tert-butyl,
and A² is selected from the group consisting of a phenyl substituted with 1, 2 or 3 substituents selected from the group consisting of methoxy, —Cl, —F, —CN, —O—(CH₂)₂-piperidinyl, —O—(CH₂)₂-morpholinyl, —O—(CH₂)₃—N(CH₃)₂, and —CH₂-morpholinyl; a phenyl or pyridinyl substituted with 2 or 3 methyl, and a pyridinyl substituted with 1, 2 or 3 substituents selected from the group consisting of methyl, —Cl, —F, —CN, —O—(CH₂)₂-piperidinyl, —O—(CH₂)₂-morpholinyl, —O—(CH₂)₃—N(CH₃)₂, and —CH₂-morpholinyl.
More particularly, A can be

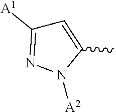

A¹ is tert-butyl,
and A² is selected from the group consisting of a phenyl substituted with 1, 2 or 3 substituents selected from the group consisting of —Cl, —F, —CN, —O—(CH₂)₂-piperidinyl, —O—(CH₂)₃—N(CH₃)₂, and —CH₂-morpholinyl;
a pyridinyl substituted with a methyl; and a phenyl substituted by two methyls.
B can be any ring as disclosed above.
In another particular aspect, the compound is represented by structure (IX) or structure (X), A is

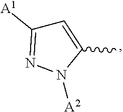

and
A¹ is selected from the group consisting of C₁-C₄ alkyl, C₃-C₆ cycloalkyl,

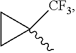

C₃-C₇ heterocycloalkyl,

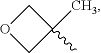

phenyl, benzyl and

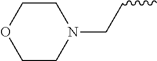

wherein A¹ is optionally fluorinated,

### A² is

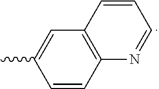

More particularly, A¹ can be selected from the group consisting of methyl, —CF₃, isopropyl, cyclopropyl, isobutyl, sec-butyl, tert-butyl,

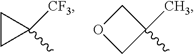

preferably A¹ is tert-butyl.
B can be any ring as disclosed above.

In an alternative aspect is provided a compound, especially for use in treating, ameliorating, delaying, curing and/or preventing a disease or condition associated with muscle cells and/or satellite cells, or a pharmaceutically acceptable salt thereof, wherein said compound is represented by structure (XVI),

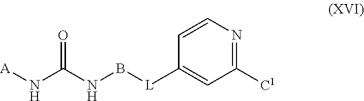

wherein C¹ is selected from the group consisting of wherein C¹ is selected from the group consisting of —H, —NH₂, —NR¹, —C(O)—NH—R¹, —NH—C(O)—R¹, —NH—S(O)₂—R¹, —NH—C(O)—OR¹, —C(O)—R¹, —R¹, —OR¹, —SO₂R¹, with R¹ being selected from the group consisting of C₁-C₁₀ alkyl, C₂-C₁₀ alkenyl, C₃-C₁₀ cycloalkyl, C₄-C₂₀ alkcycloalkyl, C₆-C₁₄ aryl, C₃-C₁₃ heteroaryl, C₇-C₂₄ alkaryl, C₃-C₇ heterocycloalkyl and C₄-C₁₇ alkheterocycloalkyl, wherein each hydrogen in each of these groups may be independently replaced by a group selected from halogen, hydroxy, —OR′, —COR′, —COOR′, and —NR′R″, each hydrogen in each of R′ and R″ may be independently replaced by —COR′″ or COOR′″, wherein R′, R″ and R′″ are independently a C₁-C₆ alkyl;
preferably C¹ is selected from the group consisting of —NH—C(O)R¹ or —NH—C(O)—OR¹ with R¹ being selected from the group consisting of C₁-C₁₀ alkyl, C₆-C₁₄ aryl, C₃-C₁₃ heteroaryl, and C₇-C₂₄ alkaryl, wherein each hydrogen in each of these groups may be independently replaced by a group selected from halogen, hydroxy, —OR′, and —NR′R″, each hydrogen in each of R′ and R″ may be independently replaced by —COR′″ or COOR′″, wherein R′, R″ and R′″ are independently a C₁-C₆ alkyl;
more preferably C¹ is selected from the group consisting of —NH—C(O)R¹ or —NH—C(O)—OR¹ with R¹ being selected from the group consisting of C₁-C₁₀ alkyl, C₆-C₁₄ aryl, C₃-C₁₃ heteroaryl, and C₇-C₂₄ alkaryl, wherein each hydrogen in each of these groups may be independently replaced by a group selected from halogen, hydroxy, and —NR′R each hydrogen in each of R′ and R″ may be independently replaced by —COR′″ or COOR′″, wherein R′, R″ and R″ are independently a C₁-C₆ alkyl.
In a particular aspect, C¹ is selected from the group consisting of

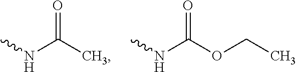

—NH—C(O)—OtBu, —NH—C(O)—N(CH₃)—C(O)—OtBu, —NH—C(O)—CH₂—N(CH₃)—C(O)—OtBu, —NH—C(O)—CH₂—NHCH₃, —NH—C(O)—Obenzyl, —NH—C(O)—CH₂—OH, preferably

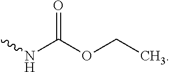

A and B can be selected according any aspect as disclosed above and any combination of A and B.
In a particular aspect, A is a ring selected from the group consisting of

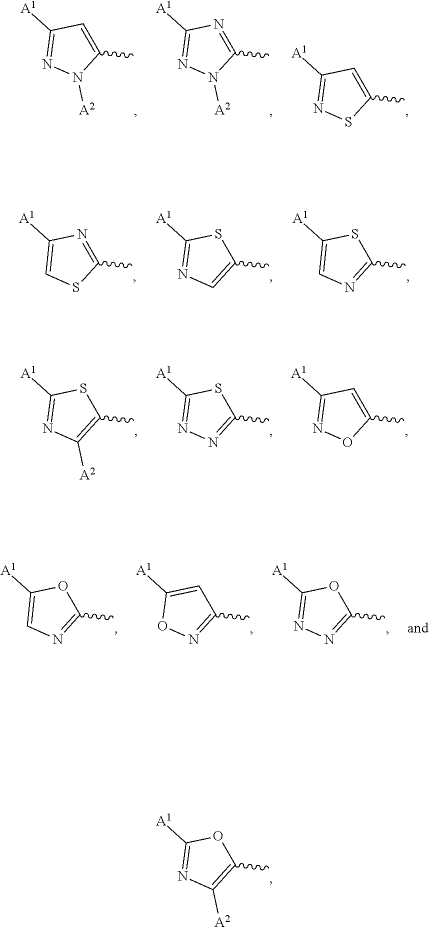

A¹ is tert-butyl,
A² is a phenyl or

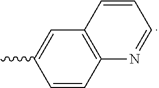

optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl.
In another particular aspect, A is a ring selected from the group consisting of

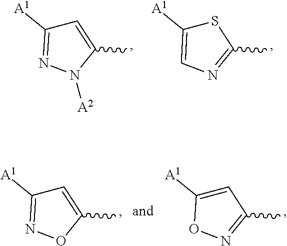

A¹ is tert-butyl,
A² is a phenyl optionally substituted with 1, 2 or 3 substituents selected from the group consisting of methyl, —Cl, —F, —CN, methoxy, —O—(CH₂)₂-piperidinyl, —O—(CH₂)₂-morpholinyl, —O—(CH₂)₃—N(CH₃)₂, and —CH₂-morpholinyl, or A² is

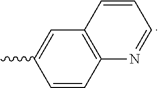

In a particular aspect, B is a ring system selected from the group consisting of

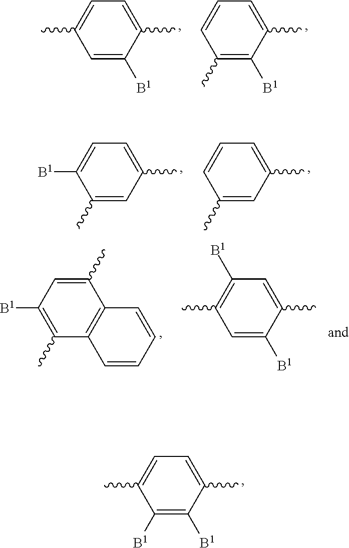

wherein, when B is a phenyl, B¹ is selected from the group consisting of —F, —Cl, —SCH₃, —SCH₂CH₃, —CH₃, —CF₃, —OCH₃, and —OCF₃ and, when B is a naphtyl, B¹ is —H.
More particularly, B is a ring system selected from the group consisting of

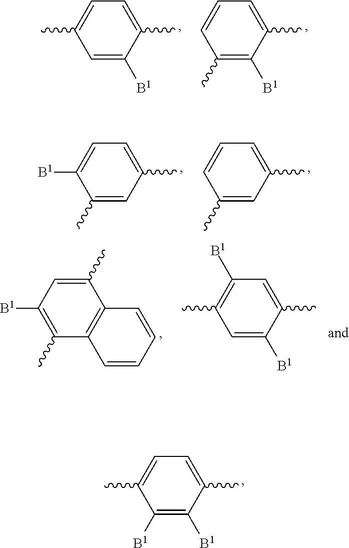

wherein, when B is a phenyl, B¹ is selected from the group consisting of —F, —SCH₃, —SCH₂CH₃, —CF₃, —OCH₃, and —OCF₃ and, when B is a naphtyl, B¹ is —H.
In an additional particular aspect, B is a ring system selected in the group consisting of

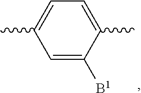

with B¹ being selected from the group consisting of —F, —SCH₃, —SCH₂CH₃, —CF₃, —OCH₃, and —OCF₃, preferably —SCH₃,

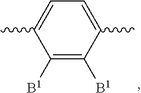

with B¹ being F,
and

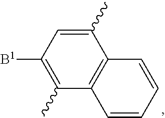

with B¹ being —H.

In a preferred embodiment is provided a compound for use in treating, ameliorating, delaying, curing and/or preventing a disease or condition associated with muscle cells and/or satellite cells, or a pharmaceutically acceptable salt thereof, wherein said compound is represented by structure (XI),

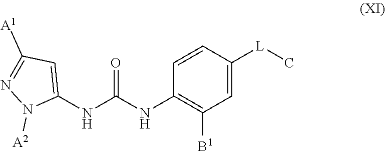

wherein A¹ is selected from the group consisting of C₁-C₄ alkyl, C₁-C₄ cycloalkyl,

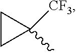

C₃-C₇ heterocycloalkyl,

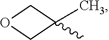

phenyl, benzyl and

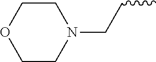

wherein A¹ is optionally fluorinated, preferably wherein A¹ is selected from the group consisting of methyl, —CF₃, isopropyl, cyclopropyl, isobutyl, sec-butyl, tert-butyl,

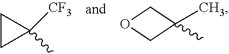

more preferably wherein A¹ is tert-butyl,
wherein A² is selected from the group consisting of H, C₁-C₄ alkyl, C₁-C₄ cycloalkyl, C₃-C₇ heterocycloalkyl, halogen, benzyl, phenyl, tolyl,

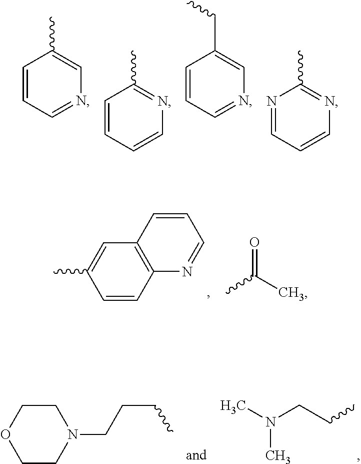

wherein A² is optionally fluorinated and optionally substituted with 1, 2 or 3 substituents selected from the group consisting of C₁-C₄ alkyl, —Cl, —F, —CN, C(O)—NR^{ABC}R^{ABC}, —OR^{ABC}, —SR^{ABC}, —NR^{ABC}R^{ABC}, —NR^{ABC}C(O)—R^{ABC}, —SO₂R^{ABC}, C₁-C₁₀ alkyl, C₃-C₁₀ cycloalkyl, C₃-C₇ heterocycloalkyl, C₃-C₁₃ heteroaryl and C₄-C₂₃ alkheteroaryl; wherein R^{ABC} is selected independently from the group consisting of C₁-C₁₀ alkyl, C₂-C₁₀ alkenyl, C₃-C₁₀ cycloalkyl, C₃-C₇ heterocycloalkyl, C₆-C₁₄ aryl, C₃-C₁₃ heteroaryl, C₇-C₂₄ alkaryl, wherein each hydrogen in each of these groups may be independently substituted by a halogen, C₃-C₇ heterocycloalkyl or NRR, with R being a C₁-C₃ alkyl, wherein B¹ is selected from the group consisting of —H, —F, —Cl, —SCH₃, —SCH₂CH₃, isopropyl, —CF₃, —OCH₃, and —OCF₃,

wherein ring system C is a monocyclic or a bicyclic ring system,
wherein said monocyclic ring system comprises a pyridine ring or a pyrimidine ring, wherein said pyridine or pyrimidine ring is optionally substituted,
preferably wherein said monocyclic system is selected from the group consisting of

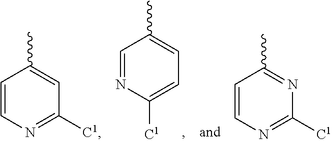

more preferably wherein said monocyclic system is

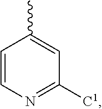

wherein C¹ is selected from the group consisting of wherein C¹ is selected from the group consisting of —H, —NH₂, —NR¹, —C(O)—NH—R¹, —NH—C(O)—R¹, —NH—S(O)₂—R¹, —NH—C(O)—OR¹, —C(O)—R¹, —R¹, —OR¹, —SO₂R¹, with R¹ being selected from the group consisting of C₁-C₁₀ alkyl, C₂-C₁₀ alkenyl, C₃-C₁₀ cycloalkyl, C₄-C₂₀ alkcycloalkyl, C₆-C₁₄ aryl, C₃-C₁₃ heteroaryl, C₇-C₂₄ alkaryl, C₃-C₇ heterocycloalkyl and C₄-C₁₇ alkheterocycloalkyl, wherein each hydrogen in each of these groups may be independently replaced by a group selected from halogen, hydroxy, —OR′, —COR′, —COOR′, and —NR′R″, each hydrogen in each of R′ and R″ may be independently replaced by —COR′″ or COOR′″, wherein R′, R″ and R′″ are independently a C₁-C₆ alkyl; wherein said bicyclic ring system comprises two nitrogen atoms, and two six-membered rings or a six-membered ring and a five-membered ring,
preferably wherein said bicyclic system is selected from the group consisting of

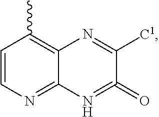

in particular

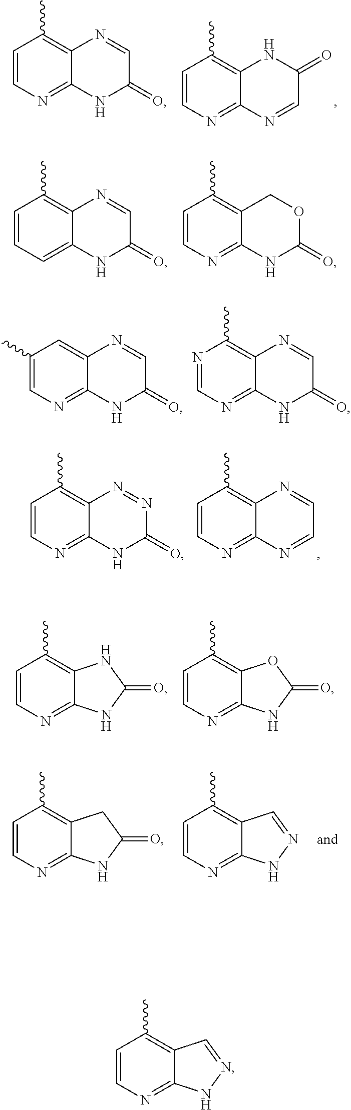

wherein C¹ is selected from the group consisting of —H, —CH₃, —OCH₃, —CN or

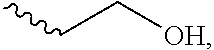

wherein L is selected from the group consisting of —O— and —S—, preferably wherein L is —O—.
In a more specific aspect, B¹ is selected from the group consisting of —CF₃, —OCF₃, —OCH₃, and SCH₂CH₃, or B has two B¹ groups selected independently from the group consisting of —F, —Cl, —SCH₃, —SCH₂CH₃, —CH₃, isopropyl, —CF₃, —OCH₃, —OCF₃ and
C is selected from the group consisting of

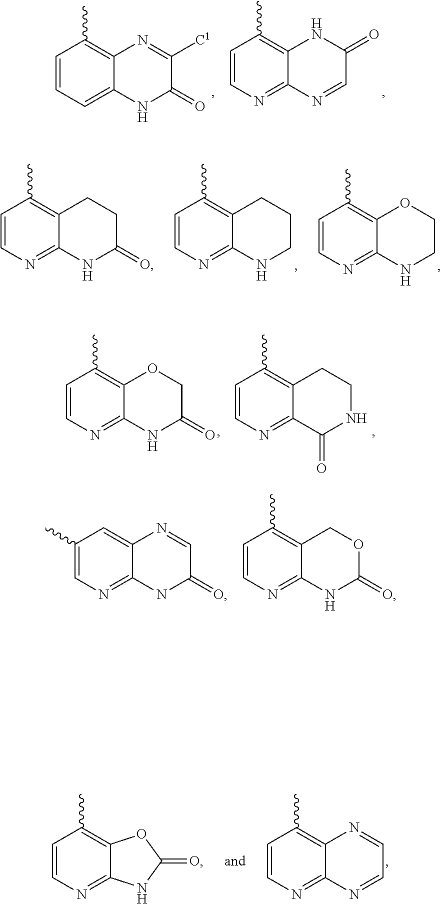

wherein C¹ is selected from the group consisting of —H, —CH₃, —OCH₃, —CN or

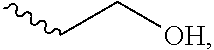

or C is selected from the group consisting of

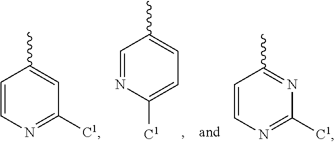

wherein C¹ is selected from the group consisting of wherein C¹ is selected from the group consisting of —H, —NH₂, —NR¹, —C(O)—NH—R¹, —NH—C(O)—R¹, —NH—S(O)₂—R¹, —NH—C(O)—OR¹, —C(O)—R¹, —R¹, —OR¹, —SO₂R¹, with R¹ being selected from the group consisting of C₁-C₁₀ alkyl, C₂-C₁₀ alkenyl, C₃-C₁₀ cycloalkyl, C₄-C₂₀ alkcycloalkyl, C₆-C₁₄ aryl, C₃-C₁₃ heteroaryl, C₇-C₂₄ alkaryl, C₃-C₇ heterocycloalkyl and C₄-C₁₇ alkheterocycloalkyl, wherein each hydrogen in each of these groups may be independently replaced by a group selected from halogen, hydroxy, —OR′, —COR′, —COOR′, and —NR′R each hydrogen in each of R′ and R″ may be independently replaced by —COR′″ or COOR′″, wherein R′, R″ and R′″ are independently a C₁-C₆ alkyl.
In another more specific aspect, B¹ is selected from the group consisting of —CF₃, —OCF₃, —OCH₃, and SCH₂CH₃, or B has two B¹ groups selected independently from the group consisting of —F, —Cl, —SCH₃, —SCH₂CH₃, —CH₃, isopropyl, —CF₃, —OCH₃, —OCF₃ and
C is selected from the group consisting of

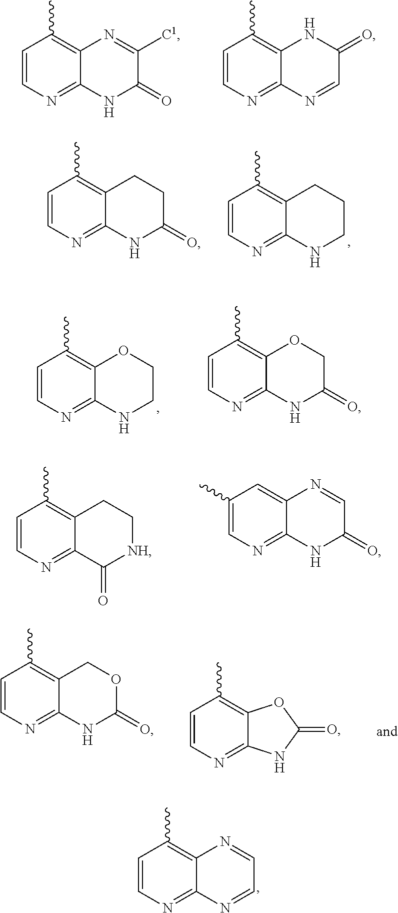

wherein C¹ is selected from the group consisting of —H, —CH₃, —OCH₃, —CN or

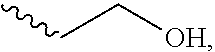

or
C is selected from the group consisting of

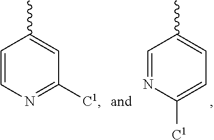

with C¹ being selected from the group consisting of —H, —NH₂,

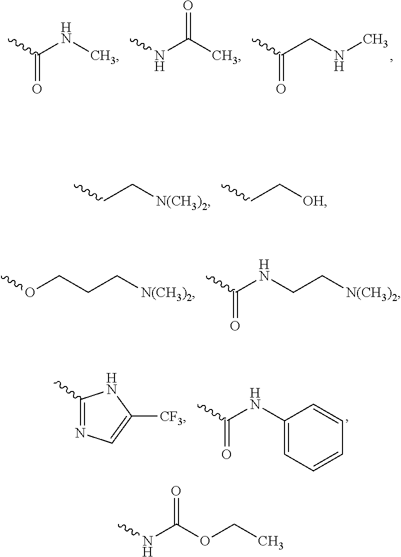

—NH—C(O)—OtBu, —NH—C(O)—N(CH₃)—C(O)—OtBu, —NH—C(O)—CH₂—N(CH₃)—C(O)—OtBu, —NH—C(O)—CH₂—NHCH₃, —NH—C(O)—Obenzyl, —NH—C(O)—CH₂—OH, —NH—(CH₂)₂—N(CH₃)₂, and —NH—S(O)₂—CH₃; preferably from the group consisting of —H,

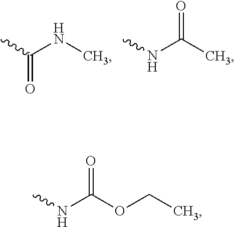

—NH—C(O)—OtBu, —NH—C(O)—N(CH₃)—C(O)—OtBu, —NH—C(O)—CH₂—N(CH₃)—C(O)—OtBu, —NH—C(O)—CH₂—NHCH₃, —NH—C(O)—Obenzyl, and —NH—C(O)—CH₂—OH.
In another more specific aspect, B¹ is selected from the group consisting of —CF₃, —OCF₃, —OCH₃, and SCH₂CH₃, or B has two B¹ groups selected independently from the group consisting of —F, —Cl, —SCH₃, —SCH₂CH₃, —CH₃, isopropyl, —CF₃, —OCH₃, —OCF₃ and
C is selected in the group consisting of

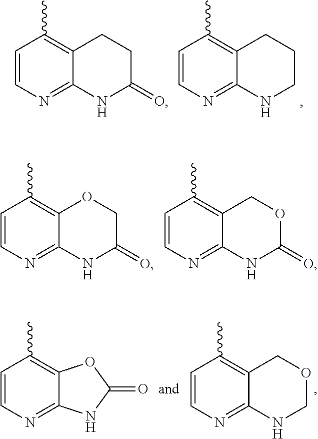

or C is selected from the group consisting of

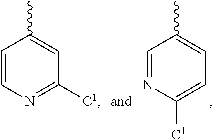

with C¹ being selected from the group consisting of

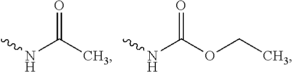

—NH—C(O)—OtBu, —NH—C(O)—CH₂—N(CH₃)—C(O)—OtBu and —NH—C(O)—N(CH₃)—CO—OtBu.
In a more specific aspect, B¹ is selected from the group consisting of —SCH₃, —CF₃, —OCF₃, —OCH₃, and SCH₂CH₃, or B has two B¹ groups selected independently from the group consisting of —F, —Cl, —SCH₃, —SCH₂CH₃, —CH₃, isopropyl, —CF₃, —OCH₃, —OCF₃ and
A¹ is selected from the group consisting of C₁-C₄ alkyl, C₁-C₄ cycloalkyl,

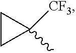

C₃-C₇ heterocycloalkyl,

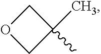

phenyl, benzyl and

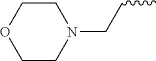

wherein A¹ is optionally fluorinated, and A² is selected from the group consisting of a phenyl substituted with 1, 2 or 3 substituents selected from the group consisting of —Cl, —F, —CN, C₁-C₃ alkyloxy optionally substituted by C₃-C₇ heterocycloalkyl or NRR, with R being a C₁-C₃ alkyl, and C₁-C₄ alkyl substituted by C₃-C₇ heterocycloalkyl or NRR, with R being a C₁-C₃ alkyl; a phenyl or pyridinyl substituted by 2 or 3 C₁-C₃ alkyl; and a pyridinyl substituted with 1, 2 or 3 substituents selected from the group consisting of —Cl, —F, —CN, C₁-C₁₀ alkyloxy substituted by C₃-C₇ heterocycloalkyl or NRR, with R being a C₁-C₃ alkyl, and C₁-C₄ alkyl substituted by C₃-C₇ heterocycloalkyl or NRR, with R being a C₁-C₃ alkyl; and
C is selected from the group consisting of

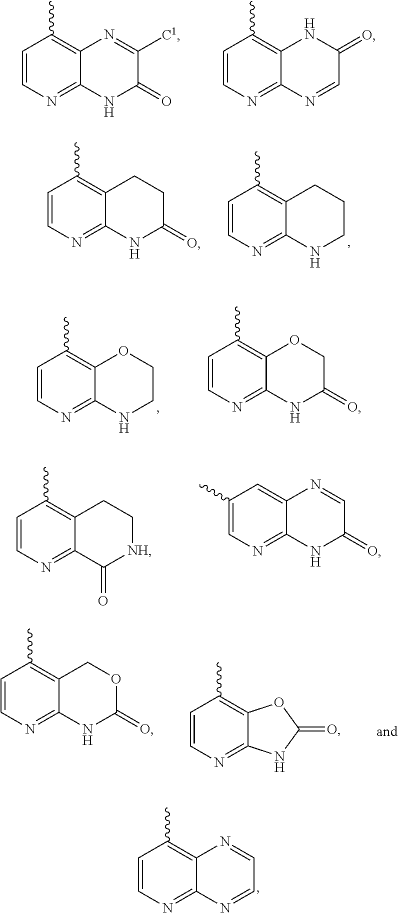

wherein C¹ is selected from the group consisting of —H, —CH₃, —OCH₃, —CN or

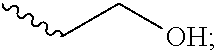

preferably from the group consisting of

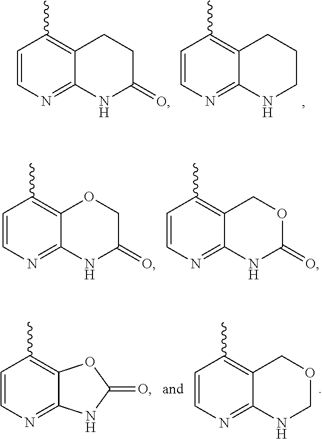

Preferably, A¹ is selected from the group consisting of methyl, —CF₃, isopropyl, cyclopropyl, isobutyl, sec-butyl, tert-butyl,

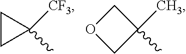

preferably tert-butyl, and A² is selected from the group consisting of a phenyl substituted with 1, 2 or 3 substituents selected from the group consisting of methoxy, —F, —CN, —O—(CH₂)₂-piperidinyl, —O—(CH₂)₂-morpholinyl, —O—(CH₂)₃—N(CH₃)₂, and —CH₂-morpholinyl; a phenyl or pyridinyl substituted with 2 or 3 methyl, and a pyridinyl substituted with 1, 2 or 3 substituents selected from the group consisting of methyl, —Cl, —F, —CN, —O—(CH₂)₂-piperidinyl, —O—(CH₂)₂-morpholinyl, —O—(CH₂)₃—N(CH₃)₂, and —CH₂-morpholinyl.
In a very specific aspect, A¹ is tert-butyl,
and A² is selected from the group consisting of a phenyl substituted with 1, 2 or 3 substituents selected from the group consisting of —Cl, —F, —CN, —O—(CH₂)₂-piperidinyl, —O—(CH₂)₃—N(CH₃)₂, and —CH₂-morpholinyl;
a pyridinyl substituted with a methyl; and a phenyl substituted by two methyl.
In a more specific aspect, B¹ is selected from the group consisting of —SCH₃, —CF₃, —OCF₃, —OCH₃, and SCH₂CH₃, or B has two B¹ groups selected independently from the group consisting of —F, —SCH₃, —SCH₂CH₃, —CH₃, isopropyl, —CF₃, —OCH₃, —OCF₃
A¹ is selected from the group consisting of methyl, —CF₃, isopropyl, cyclopropyl, isobutyl, sec-butyl, tert-butyl,

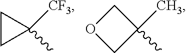

preferably A¹ is tert-butyl,

### A² is

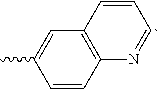

and
C is selected from the group consisting of

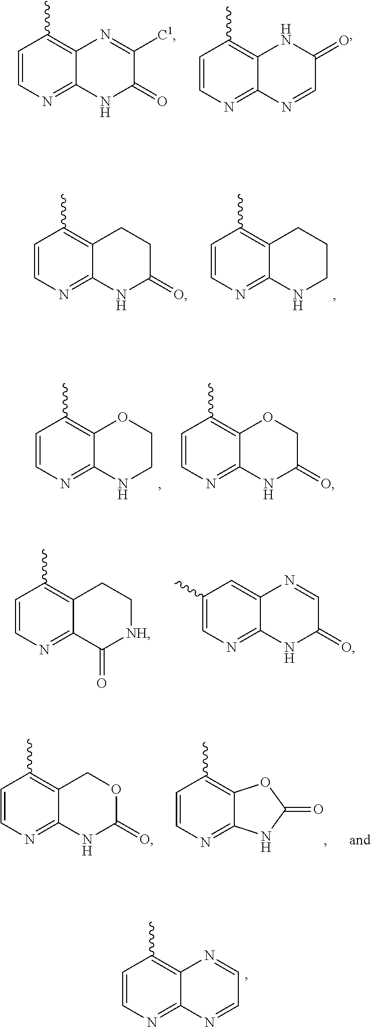

wherein C¹ is selected from the group consisting of —H, —CH₃, —OCH₃, —CN or

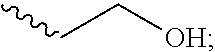

preferably C is selected from the group consisting of

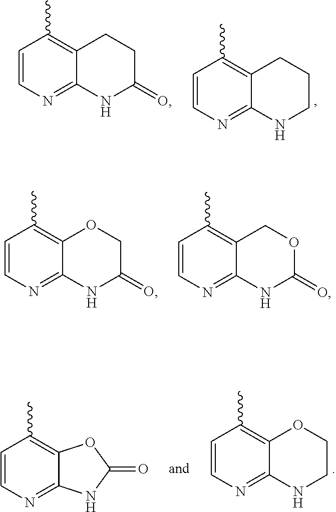

In a more specific aspect, B¹ is selected from the group consisting of —SCH₃, —CF₃, —OCF₃, —OCH₃, and SCH₂CH₃, or B has two B¹ groups selected independently from the group consisting of —F, —Cl, —SCH₃, —SCH₂CH₃, —CH₃, isopropyl, —CF₃, —OCH₃, —OCF₃,
A¹ is selected from the group consisting of methyl, —CF₃, isopropyl, cyclopropyl, isobutyl, sec-butyl, tert-butyl,

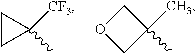

A² is selected from the group consisting of H, methyl, iso-propyl, benzyl, phenyl, chlorine,

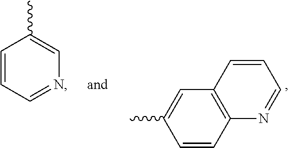

wherein A¹ and A² are optionally fluorinated, and A² is optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl, and
C is selected from the group consisting of

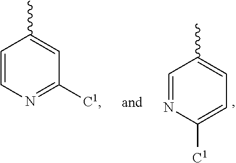

with C¹ being selected from the group consisting of wherein C¹ is selected from the group consisting of —H, —NH₂, —NR¹, —C(O)—NH—R¹, —NH—C(O)—R¹, —NH—S(O)₂—R¹, —NH—C(O)—OR¹, —C(O)—R¹, —R¹, —OR¹, —SO₂R¹, with R¹ being selected from the group consisting of C₁-C₁₀ alkyl, C₆-C₁₄ aryl, C₃-C₁₃ heteroaryl, and C₇-C₂₄ alkaryl, wherein each hydrogen in each of these groups may be independently replaced by a group selected from a halogen, a hydroxy, —OR′, and —NR′R″, each hydrogen in each of R′ and R″ may be independently replaced by —COR′″ or COOR′″, wherein R′, R″ and R′″ are independently a C₁-C₆ alkyl.
Optionally, C¹ can be selected from the group consisting of —NH—C(O)R¹ or —NH—C(O)—OR¹ with R¹ being selected from the group consisting of C₁-C₁₀ alkyl, C₆-C₁₄ aryl, C₃-C₁₃ heteroaryl, and C₇-C₂₄ alkaryl, wherein each hydrogen in each of these groups may be independently replaced by a group selected from halogen, hydroxy, —OR′, and —NR′R″, each hydrogen in each of R′ and R″ may be independently replaced by —COR′″ or COOR′″, wherein R′, R″ and R′″ are independently a C₁-C₆ alkyl.
More specifically, A¹ is tert-butyl,
A² is a phenyl or

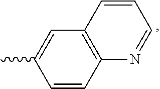

optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl, and
C is selected from the group consisting of

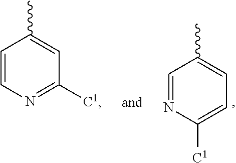

with C¹ being selected from the group consisting of —H, —NH₂,

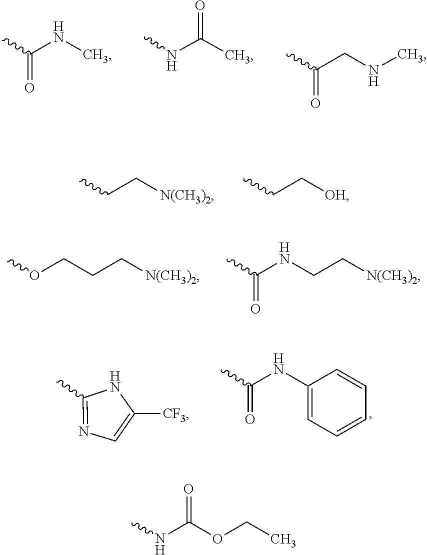

—NH—C(O)—OtBu, —NH—C(O)—N(CH₃)—C(O)—OtBu, —NH—C(O)—CH₂—N(CH₃)—C(O)—OtBu, —NH—C(O)—CH₂—NHCH₃, —NH—C(O)—Obenzyl, —NH—C(O)—CH₂—OH, —NH—(CH₂)₂—N(CH₃)₂, and —NH—S(O)₂—CH₃; preferably from the group consisting of —H,

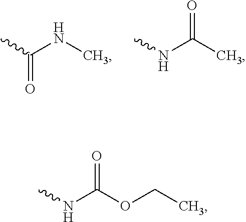

—NH—C(O—)OtBu, —NH—C(O)—N(CH₃)—C(O)—OtBu, —NH—C(O)—CH₂—N(CH₃)—C(O)—OtBu, —NH—C(O)—CH₂—NHCH₃, —NH—C(O)—Obenzyl, and —NH—C(O)—CH₂—OH.
More specifically, A¹ is tert-butyl,
A² is a phenyl optionally substituted with 1, 2 or 3 substituents selected from the group consisting of methyl, —Cl, —F, —CN, methoxy, —O—(CH₂)₂-piperidinyl, —O—(CH₂)₂-morpholinyl, —O—(CH₂)₃—N(CH₃)₂, and —CH₂-morpholinyl, or A² is

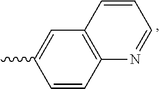

and
C is selected from the group consisting of

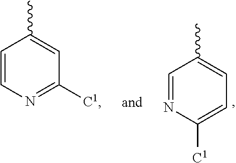

with C¹ being selected from the group consisting of

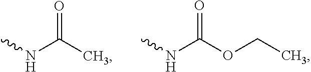

—NH—C(O—)OtBu, —NH—C(O)—CH₂—N(CH₃)—C(O)—OtBu and —NH—C(O)—N(CH₃)—CO—OtBu.
More specifically, B¹ is —SCH₃ in any of these specific aspects.
In a particular aspect, is provided a compound represented in formula (XI), wherein
wherein A¹ is selected from the group consisting of C₁-C₄ alkyl, C₁-C₄ cycloalkyl,

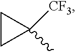

C₃-C₇ heterocycloalkyl,

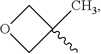

phenyl, benzyl and

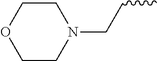

wherein A¹ is optionally fluorinated, preferably wherein A¹ is selected from the group consisting of methyl, —CF₃, isopropyl, cyclopropyl, isobutyl, sec-butyl, tert-butyl,

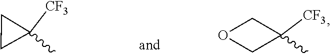

more preferably wherein A¹ is tert-butyl,
wherein A² is selected from the group consisting of H, C₁-C₄ alkyl, C₁-C₄ cycloalkyl, benzyl, phenyl, tolyl,

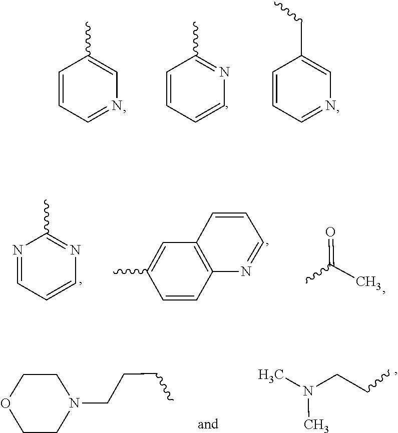

wherein A² is optionally substituted with a substituent selected from the group consisting of C₁-C₄ alkyl, —Cl, —F, —CN, C(O)NR^{ABC}R^{ABC}, —OR^{ABC}, —SR^{ABC}, —NR^{ABC}R^{ABC}, —NR^{ABC}C(O)—R^{ABC}, C₁-C₁₀ alkyl, C₃-C₁₀ cycloalkyl, C₃-C₇ heterocycloalkyl, C₃-C₁₃ heteroaryl and C₄-C₂₃ alkheteroaryl; wherein R^{ABC} is selected independently from the group consisting of C₁-C₁₀ alkyl, C₂-C₁₀ alkenyl, C₃-C₁₀ cycloalkyl, C₃-C₇ heterocycloalkyl, C₆-C₁₄ aryl, C₃-C₁₃ heteroaryl, C₇-C₂₄ alkaryl, wherein each hydrogen in each of these groups may be independently substituted by a halogen, preferably wherein A₂ is selected from the group consisting of H, methyl, iso-propyl, benzyl, phenyl, chlorine,

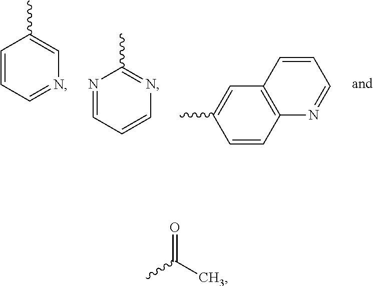

more preferably wherein A² is phenyl;
wherein B¹ is selected from the group consisting of —H, —F, —SCH₃, —SCH₂CH₃, isopropyl, —CF₃; preferably wherein B¹ is selected from the group consisting of —F, —CF₃ and —SCH₃, most preferably wherein B¹ is selected from the group consisting of —F and —SCH₃;
wherein ring system C is a monocyclic or a bicyclic ring system,
wherein said monocyclic ring system comprises a pyridine ring, wherein said pyridine ring is optionally substituted,
preferably wherein said monocyclic system is selected from the group consisting of

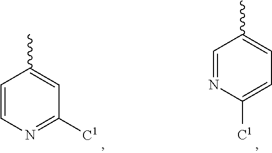

more preferably wherein said monocyclic system is

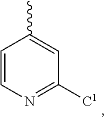

wherein C¹ is selected from the group consisting of —H, —NH₂,

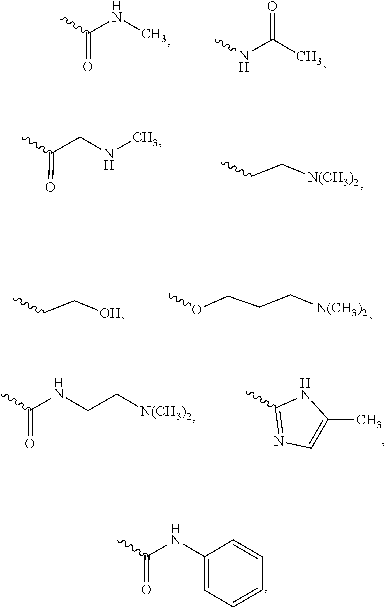

more preferably wherein C¹ is selected from the group consisting of

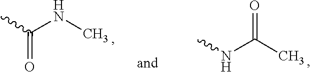

most preferably wherein C¹ is

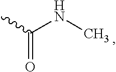

wherein said bicyclic ring system comprises two nitrogen atoms, and two six-membered rings or a six-membered ring and a five-membered ring,
preferably wherein said bicyclic system is selected from the group consisting of

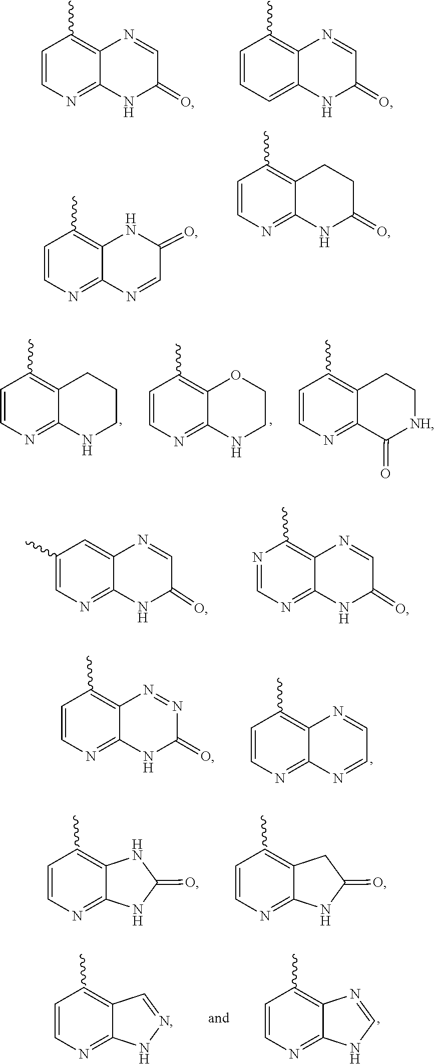

wherein said bicyclic system is optionally substituted with —OCH₃ or —CN,

optionally wherein said bicyclic system is

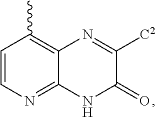

wherein C² is selected from the group consisting of —H, —OCH₃ and —CN, most preferably wherein C² is —H,
wherein L is selected from the group consisting of —O— and —S—, preferably wherein L is —O—.
In a related embodiment is provided a compound according to the invention wherein C is

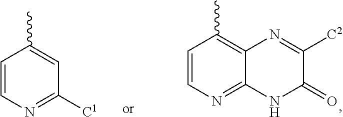

wherein C¹ and C² are defined as above. It is further preferred that L is O.

In an alternative aspect is provided a compound especially for use in treating, ameliorating, delaying, curing and/or preventing a disease or condition associated with muscle cells and/or satellite cells, or a pharmaceutically acceptable salt thereof, wherein said compound is represented by structure (XI),

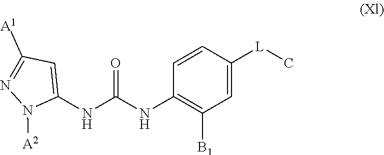

wherein A¹ is selected from the group consisting of C₁-C₄ alkyl, C₁-C₄ cycloalkyl,

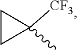

C₃-C₇ heterocycloalkyl,

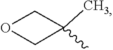

phenyl, benzyl and

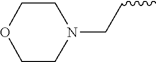

wherein A¹ is optionally fluorinated,
preferably wherein A¹ is selected from the group consisting of methyl, —CF₃, isopropyl, cyclopropyl, isobutyl, sec-butyl, tert-butyl,

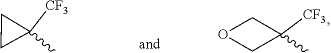

more preferably wherein A¹ is tert-butyl,
wherein A² is selected from the group consisting of H, C₁-C₄ alkyl, C₁-C₄ cycloalkyl, benzyl, phenyl, tolyl,

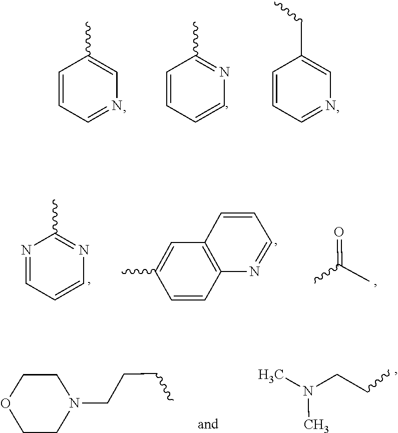

wherein A² is optionally substituted with a substituent selected from the group consisting of C₁-C₄ alkyl, —Cl, —F, —CN, C(O)NR^{ABC}R^{ABC}, —OR^{ABC}, —SR^{ABC}, —NR^{ABC}R^{ABC}, —NR^{ABC}C(O)—R^{ABC}, C₁-C₁₀ alkyl, C₃-C₁₀ cycloalkyl, C₃-C₇ heterocycloalkyl, C₃-C₁₃ heteroaryl and C₄-C₂₃ alkheteroaryl; wherein RAK is selected independently from the group consisting of C₁-C₁₀ alkyl, C₂-C₁₀ alkenyl, C₃-C₁₀ cycloalkyl, C₃-C₇ heterocycloalkyl, C₆-C₁₄ aryl, C₃-C₁₃ heteroaryl, C₇-C₂₄ alkaryl, wherein each hydrogen in each of these groups may be independently substituted by a halogen, preferably wherein A² is selected from the group consisting of H, methyl, iso-propyl, benzyl, phenyl, chlorine,

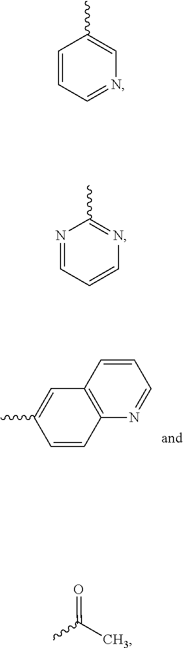

more preferably wherein A² is phenyl;
wherein B¹ is selected from the group consisting of —H, —F, —SCH₃, —SCH₂CH₃, isopropyl, —CF₃; preferably wherein B¹ is selected from the group consisting of —F, —CF₃ and —SCH₃, most preferably wherein B¹ is selected from the group consisting of —F and —SCH₃;
wherein C is a monocyclic ring system comprising a pyridine ring, wherein said pyridine ring is optionally substituted,
preferably wherein said monocyclic system is selected from the group consisting of

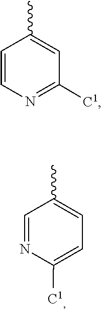

more preferably wherein said monocyclic system is

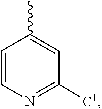

wherein C¹ is selected from the group consisting of —H, —NH₂,

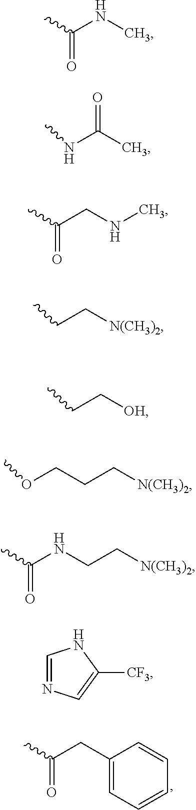

more preferably wherein C¹ is selected from the group consisting of

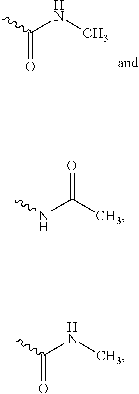

most preferably wherein C¹ is
wherein L is selected from the group consisting of —O— and —S—, preferably wherein L is —O—.

In a preferred embodiment is provided a compound especially for use in treating, ameliorating, delaying, curing and/or preventing a disease or condition associated with muscle cells and/or satellite cells, or a pharmaceutically acceptable salt thereof, wherein said compound is represented by structure (XII),

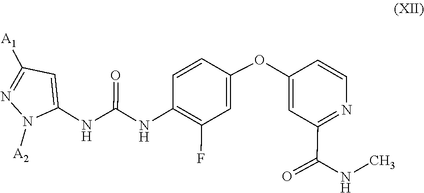

wherein A¹ is selected from the group consisting of C₁-C₄ alkyl, C₁-C₄ cycloalkyl,

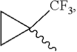

C₃-C₇ heterocycloalkyl,

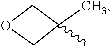

phenyl, benzyl and

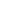

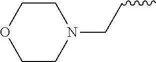

wherein A¹ is optionally fluorinated,
preferably wherein A¹ is selected from the group consisting of methyl, —CF₃, isopropyl, cyclopropyl, isobutyl, sec-butyl, tert-butyl,

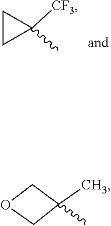

more preferably wherein A¹ is tert-butyl,
wherein A² is selected from the group consisting of H, C₁-C₄ alkyl, C₁-C₄ cycloalkyl, benzyl, phenyl, tolyl,

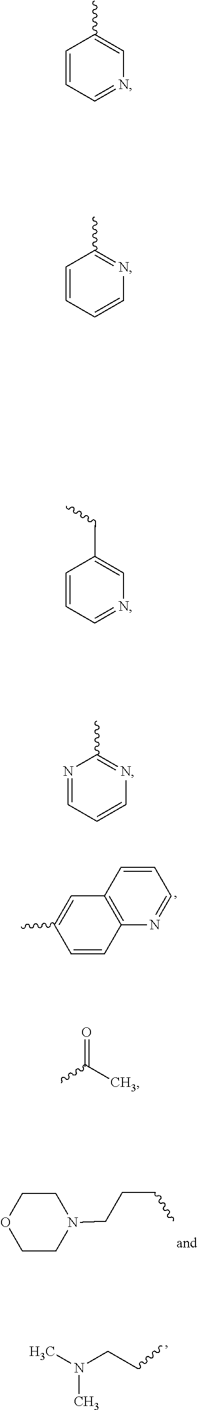

wherein A² is optionally substituted with a substituent selected from the group consisting of C₁-C₄ alkyl, —Cl, —F, —CN, C(O)—NR^{ABC}R^{ABC}, —OR^{ABC}, —SR^{ABC}, —NR^{ABC}R^{ABC}, —NR^{ABC}C(O)—R^{ABC}, C₁-C₁₀ alkyl, C₃-C₁₀ cycloalkyl, C₃-C₇ heterocycloalkyl, C₃-C₁₃ heteroaryl and C₄-C₂₃ alkheteroaryl; wherein R^{ABC} is selected independently from the group consisting of C₁-C₁₀ alkyl, C₂-C₁₀ alkenyl, C₃-C₁₀ cycloalkyl, C₃-C₇ heterocycloalkyl, C₆-C₁₄ aryl, C₃-C₁₃ heteroaryl, C₇-C₂₄ alkaryl, wherein each hydrogen in each of these groups may be independently substituted by a halogen, preferably wherein A² is selected from the group consisting of H, methyl, iso-propyl, benzyl, phenyl, chlorine,

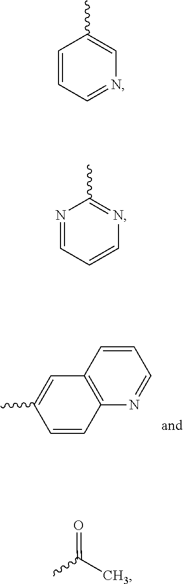

more preferably wherein A² is phenyl.

In a preferred embodiment is provided a compound especially for use in treating, ameliorating, delaying, curing and/or preventing a disease or condition associated with muscle cells and/or satellite cells, or a pharmaceutically acceptable salt thereof, wherein said compound is represented by structure (XIII),

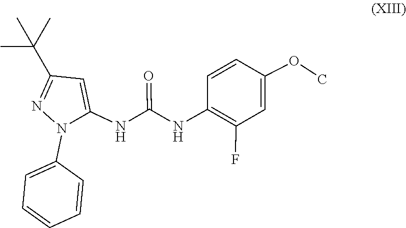

wherein C is a monocyclic ring system comprising a pyridine ring, wherein said pyridine ring is optionally substituted,
preferably wherein said monocyclic system is selected from the group consisting of

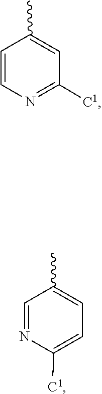

more preferably wherein said monocyclic system is

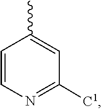

wherein C¹ is selected from the group consisting of

### 1) —H, —NH₂,

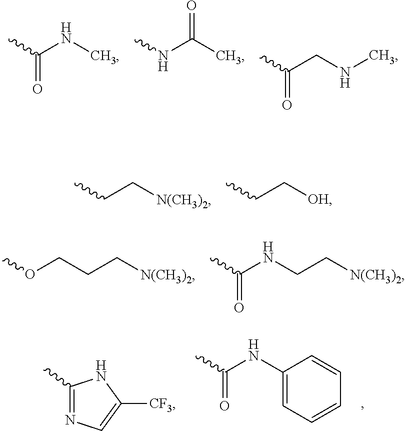

more preferably wherein C¹ is selected from the group consisting of

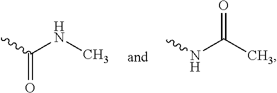

most preferably wherein C¹ is

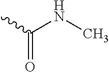

or

2) C¹ is selected from the group consisting of —NH—C(O)R¹ or —NH—C(O)—OR¹ with R¹ being selected from the group consisting of C₁-C₁₀ alkyl, C₆-C₁₄ aryl, C₃-C₁₃ heteroaryl, and C₇-C₂₄ alkaryl, wherein each hydrogen in each of these groups may be independently replaced by a group selected from halogen, hydroxy, —OR′, and —NR′R″, each hydrogen in each of R′ and R″ may be independently replaced by —COR′″ or COOR′″, wherein R′, R″ and R′″ are independently a C₁-C₆ alkyl, preferably from the group consisting of

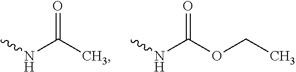

—NH—C(O)—OtBu, —NH—C(O)—N(CH₃)—C(O)—OtBu, —NH—C(O)—CH₂—N(CH₃)—C(O)—OtBu, —NH—C(O)—CH₂—NHCH₃, —NH—C(O)—Obenzyl, —NH—C(O)—CH₂—OH, preferably

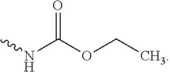

In an aspect of the disclosure is provided a compound for use in treating, ameliorating, delaying, curing and/or preventing a disease or condition associated with muscle cells and/or satellite cells, or a pharmaceutically acceptable salt thereof, wherein said compound is represented by structure (XI),

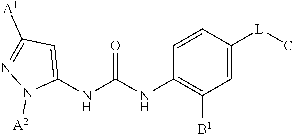

wherein A¹ is selected from the group consisting of C₁-C₄ alkyl, C₁-C₄ cycloalkyl,

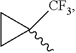

C₃-C₇ heterocycloalkyl,

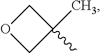

phenyl, benzyl and

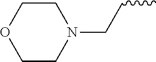

wherein A¹ is optionally fluorinated,
preferably wherein A¹ is selected from the group consisting of methyl, —CF₃, isopropyl, cyclopropyl, isobutyl, sec-butyl, tert-butyl,

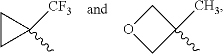

more preferably wherein A¹ is tert-butyl,
wherein A² is selected from the group consisting of H, C₁-C₄ alkyl, C₁-C₄ cycloalkyl, benzyl, phenyl, tolyl,

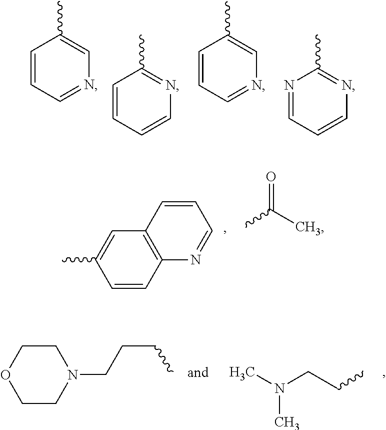

wherein A² is optionally substituted with a substituent selected from the group consisting of C₁-C₄ alkyl, —Cl, —F, —CN, C(O)—NR^{ABC}R^{ABC}, —OR^{ABC}, —SR^{ABC}, —NR^{ABC}R^{ABC}, —NR^{ABC}C(O)—R^{ABC}, C₁-C₁₀alkyl, C₃-C₁₀ cycloalkyl, C₃-C₇ heterocycloalkyl, C₃-C₁₃ heteroaryl and C₄-C₂₃ alkheteroaryl; wherein R^{ABC} is selected independently from the group consisting of C₁-C₁₀ alkyl, C₂-C₁₀ alkenyl, C₃-C₁₀ cycloalkyl, C₃-C₇ heterocycloalkyl, C₆-C₁₄ aryl, C₃-C₁₃ heteroaryl, C₇-C₂₄ alkaryl, wherein each hydrogen in each of these groups may be independently substituted by a halogen, preferably wherein A² is selected from the group consisting of H, methyl, iso-propyl, benzyl, phenyl, chlorine,

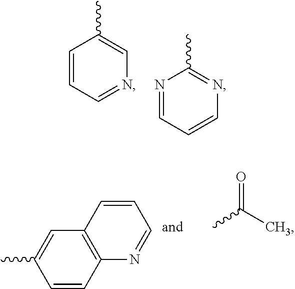

more preferably wherein A² is phenyl;
wherein B¹ is selected from the group consisting of —H, —F, —SCH₃, —SCH₂CH₃, isopropyl, —CF₃; preferably wherein B¹ is selected from the group consisting of —F, —CF₃ and —SCH₃, most preferably wherein B₁ is selected from the group consisting of —F and —SCH₃;
wherein C is a bicyclic ring system comprising two nitrogen atoms, and two six-membered rings or a six-membered ring and a five-membered ring,
preferably wherein said bicyclic system is selected from the group consisting of

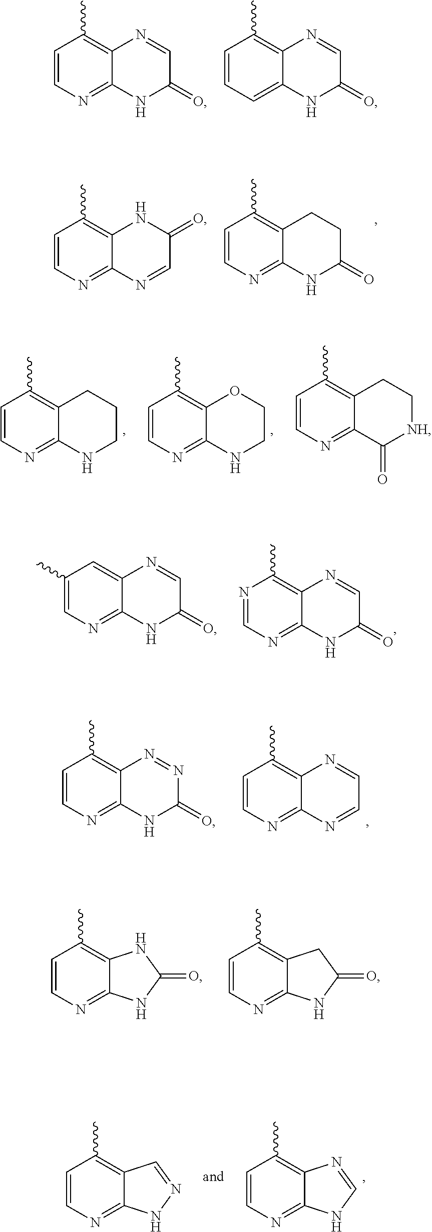

wherein said bicyclic system is optionally substituted with —OCH₃ or —CN,
optionally said bicyclic system is

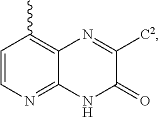

wherein C² is selected from the group consisting of —H, —OCH₃ and —CN, most preferably wherein C² is —H,
wherein L is selected from the group consisting of —O— and —S—, preferably wherein L is —O—.

In a preferred embodiment is provided a compound for use in treating, ameliorating, delaying, curing and/or preventing a disease or condition associated with muscle cells and/or satellite cells, or a pharmaceutically acceptable salt thereof, wherein said compound is represented by structure (XV),

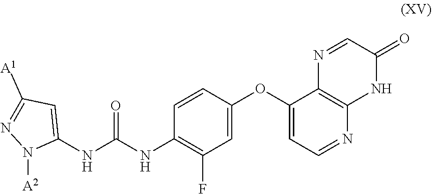

wherein A¹ is selected from the group consisting of C₁-C₄ alkyl, C₁-C₄ cycloalkyl,

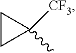

C₃-C₇ heterocycloalkyl,

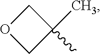

phenyl, benzyl and

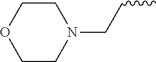

wherein A¹ is optionally fluorinated,
preferably A¹ is selected from the group consisting of methyl, —CF₃, isopropyl, cyclopropyl, isobutyl, sec-butyl, tert-butyl,

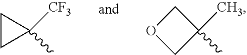

preferably A¹ is isobutyl or tert-butyl, still more preferably A¹ is tert-butyl,
wherein A² is selected from the group consisting of H, C₁-C₄ alkyl, C₁-C₄ cycloalkyl, benzyl, phenyl, tolyl,

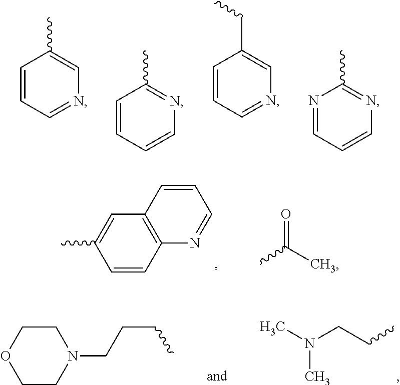

wherein A² is optionally substituted with a substituent selected from the group consisting of C₁-C₄ alkyl, —Cl, —F, —CN, C(O)—NR^{ABC}R^{ABC}, —OR^{ABC}, —SR^{ABC}, —NR^{ABC}R^{ABC}, —NR^{ABC}C(O)—R^{ABC}, C₁-C₁₀alkyl, C₃-C₁₀ cycloalkyl, C₃-C₇ heterocycloalkyl, C₃-C₁₃ heteroaryl and C₄-C₂₃ alkheteroaryl; wherein R^{ABC} is selected independently from the group consisting of C₁-C₁₀ alkyl, C₂-C₁₀ alkenyl, C₃-C₁₀ cycloalkyl, C₃-C₇ heterocycloalkyl, C₆-C₁₄ aryl, C₃-C₁₃ heteroaryl, C₇-C₂₄ alkaryl, wherein each hydrogen in each of these groups may be independently substituted by a halogen, preferably A₂ is selected from the group consisting of H, methyl, iso-propyl, benzyl, phenyl, chlorine,

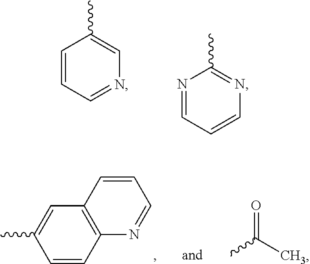

more preferably A² is phenyl,

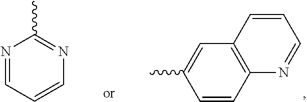

still more preferably a phenyl.
In a particular aspect of the compound of formula (XV), A¹ is isobutyl or tert-butyl, preferably A¹ is tert-butyl, and
A² is phenyl,

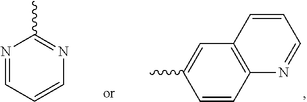

preferably

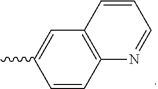

In a preferred embodiment is provided a compound especially for use in treating, ameliorating, delaying, curing and/or preventing a disease or condition associated with muscle cells and/or satellite cells, or a pharmaceutically acceptable salt thereof, wherein said compound is represented by structure (XI),

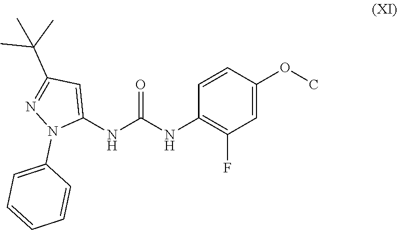

wherein C is a bicyclic ring system comprising two nitrogen atoms, and two six-membered rings or a six-membered ring and a five-membered ring,
1) C is selected from the group consisting of

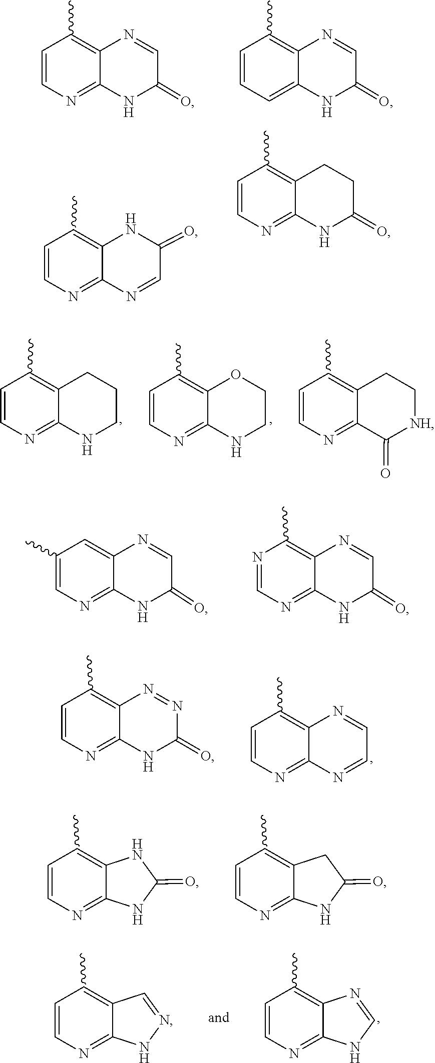

wherein said bicyclic system is optionally substituted with —OCH₃ or —CN,
optionally said bicyclic system is

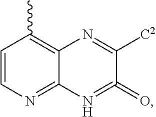

wherein C² is selected from the group consisting of —H, —OCH₃ and —CN, most preferably wherein C² is —H;

or

2) C is selected from the group consisting of

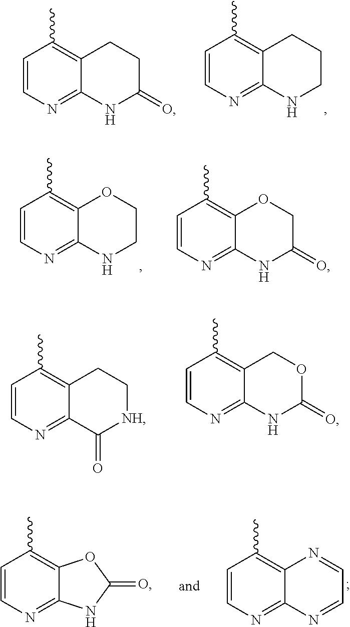

preferably

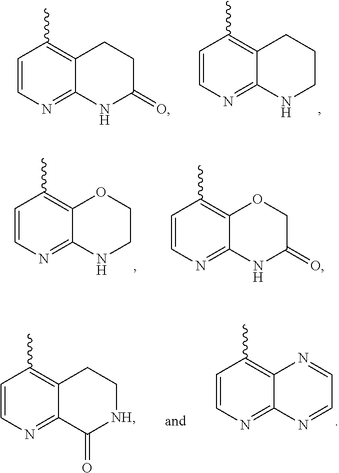

In a preferred embodiment is provided a compound especially for use in treating, ameliorating, delaying, curing and/or preventing a disease or condition associated with muscle cells and/or satellite cells, or a pharmaceutically acceptable salt thereof, wherein said compound is represented by structure (XV),

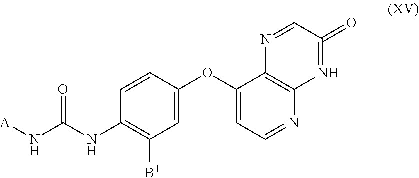

wherein A is a ring system comprising a five-membered or a six-membered heteroaromatic ring, wherein said ring system does not comprise more than 30 carbon atoms;
wherein B¹ is selected from the group consisting of —H, —F, —SCH₃, —SCH₂CH₃, isopropyl, —CF₃, preferably B¹ is selected from the group consisting of —F and —SCH₃, most preferably B¹ is —F.
In a particular aspect of the compound of formula (XV),
1)

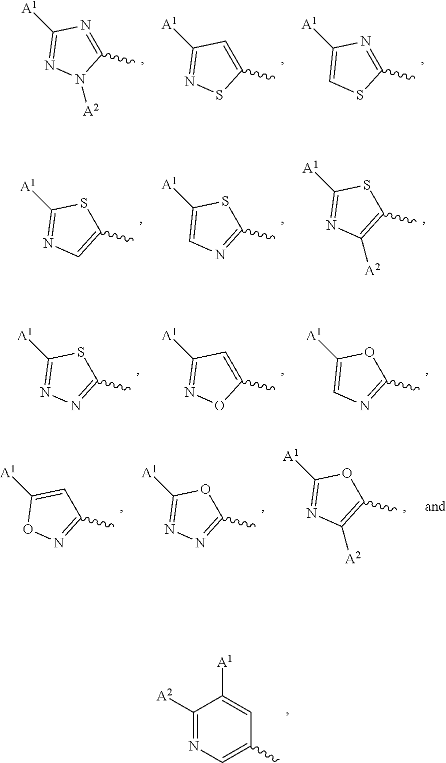

wherein A¹ is selected from the group consisting of C₁-C₄ alkyl, C₁-C₄ cycloalkyl,

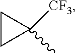

C₃-C₇ heterocycloalkyl,

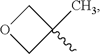

phenyl, benzyl and

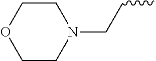

wherein A¹ is optionally fluorinated, and A² is selected from the group consisting of —H, C₁-C₄ alkyl, C₃-C₆ cycloalkyl, C₃-C₇ heterocycloalkyl, halogen, benzyl, phenyl, tolyl,

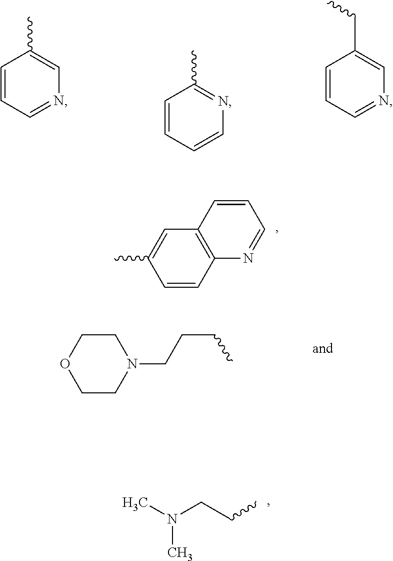

wherein A¹ and A² are optionally fluorinated, and A₂ is optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,
or
2)
B¹ is selected from the group consisting of —CF₃, —OCF₃, —OCH₃, and SCH₂CH₃,
or
3)

### A is

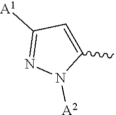

wherein A¹ is selected from the group consisting of C₁-C₄ alkyl, C₁-C₄ cycloalkyl,

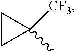

C₃-C₇ heterocycloalkyl,

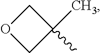

phenyl, benzyl and

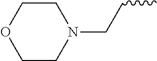

wherein A¹ is optionally fluorinated,
and A² is selected from the group consisting of a phenyl substituted with 1, 2 or 3 substituents selected from the group consisting of —Cl, —F, —CN, C₁-C₃ alkyloxy optionally substituted by C₃-C₇ heterocycloalkyl or NRR, with R being a C₁-C₃ alkyl, and C₁-C₄ alkyl substituted by C₃-C₇ heterocycloalkyl or NRR, with R being a C₁-C₃ alkyl; a phenyl or pyridinyl substituted by 2 or 3 C₁-C₃ alkyl; and a pyridinyl substituted with 1, 2 or 3 substituents selected from the group consisting of —Cl, —F, —CN, C₁-C₁₀ alkyloxy substituted by C₃-C₇ heterocycloalkyl or NRR, with R being a C₁-C₃ alkyl, and C₁-C₄ alkyl substituted by C₃-C₇ heterocycloalkyl or NRR, with R being a C₁-C₃ alkyl;
or
4)

### A is

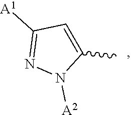

and
A¹ is selected from the group consisting of C₁-C₄ alkyl, C₃-C₆ cycloalkyl,

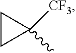

C₃-C₇ heterocycloalkyl,

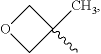

phenyl, benzyl and

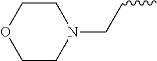

wherein A¹ is optionally fluorinated,

### A² is

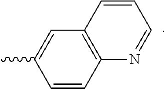

In a more specific particular aspect of the compound of formula (XV),
1)
A is a ring selected from the group consisting of

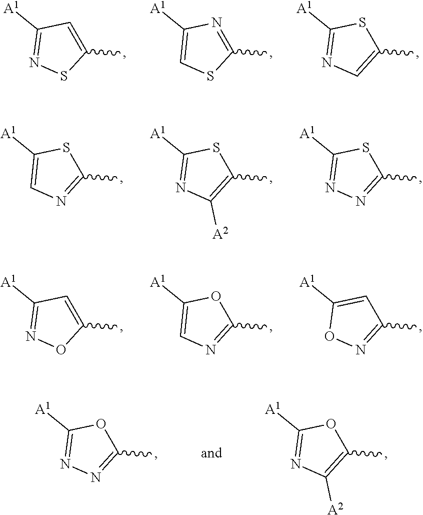

A¹ is selected from the group consisting of methyl, —CF₃, isopropyl, cyclopropyl, isobutyl, sec-butyl, tert-butyl,

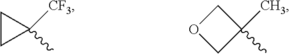

### A² is —H,

or
2)
B¹ is selected from the group consisting of —CF₃, and —OCF₃,
or
3)

### A is

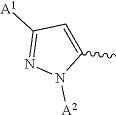

wherein A¹ is selected from the group consisting of C₁-C₄ alkyl, C₁-C₄ cycloalkyl,

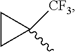

C₃-C₇ heterocycloalkyl,

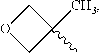

phenyl, benzyl and

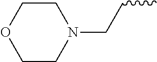

wherein A¹ is optionally fluorinated, preferably tert-butyl;
and A² is selected from the group consisting of a phenyl substituted with 1, 2 or 3 substituents selected from the group consisting of methoxy, —Cl, —F, —CN, —O—(CH₂)₂-piperidinyl, —O—(CH₂)₂-morpholinyl, —O—(CH₂)₃—N(CH₃)₂, and —CH₂-morpholinyl; a phenyl or pyridinyl substituted with 2 or 3 methyl, and a pyridinyl substituted with 1, 2 or 3 substituents selected from the group consisting of methyl, —Cl, —F, —CN, —O—(CH₂)₂-piperidinyl, —O—(CH₂)₂-morpholinyl, —O—(CH₂)₃—N(CH₃)₂, and —CH₂-morpholinyl; preferably from the group consisting of A² is selected from the group consisting of a phenyl substituted with 1, 2 or 3 substituents selected from the group consisting of methoxy, —Cl, —F, —CN, —O—(CH₂)₂-piperidinyl, —O—(CH₂)₂-morpholinyl, —O—(CH₂)₃—N(CH₃)₂, and —CH₂-morpholinyl; a phenyl or pyridinyl substituted with 2 or 3 methyl, and a pyridinyl substituted with 1, 2 or 3 substituents selected from the group consisting of methyl, —Cl, —F, —CN, —O—(CH₂)₂-piperidinyl, —O—(CH₂)₂-morpholinyl, —O—(CH₂)₃—N(CH₃)₂, and —CH₂-morpholinyl; still more preferably from the group consisting of a phenyl substituted with 1, 2 or 3 substituents selected from the group consisting of —Cl, —F, —CN, —O—(CH₂)₂-piperidinyl, —O—(CH₂)₃—N(CH₃)₂, and —CH₂-morpholinyl; a pyridinyl substituted with a methyl; and a phenyl substituted by two methyl;
or
4)

### A is

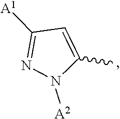

and
A¹ is selected from the group consisting of C₁-C₄ alkyl, C₃-C₆ cycloalkyl,

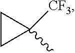

C₃-C₇ heterocycloalkyl,

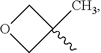

phenyl, benzyl and

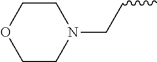

wherein A¹ is optionally fluorinated, preferably tert-butyl;

### A² is

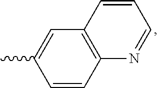

The invention also relates to a compound represented by a structure as defined below in table 1 or a pharmaceutically acceptable salt thereof. The invention also relates to a compound represented by a structure as defined below in table 1, or a pharmaceutically acceptable salt, or a pharmaceutical or veterinary composition comprising such a compound for use as a medicament, preferably for use in treating, ameliorating, delaying, curing and/or preventing a disease or condition associated with muscle cells and/or satellite cells.

The present invention further relates to the use of a compound represented by a structure as defined below in table 1 or a pharmaceutically acceptable salt thereof for the manufacture of a medicament, in particular for treating, ameliorating, delaying, curing and/or preventing a disease or condition associated with muscle cells and/or satellite cells.

The present invention also relates to a method for treating, ameliorating, delaying, curing and/or preventing a disease or condition associated with muscle cells and/or satellite cells in a subject in need thereof comprising administering a therapeutically effective amount of a compound represented by a structure as defined below in table 1 or a pharmaceutically acceptable salt thereof, thereby treating, ameliorating, delaying, curing and/or preventing a disease or condition associated with muscle cells and/or satellite cells.

TABLE 1

| Compound | Structure | Name |
|---|---|---|
| (a) | 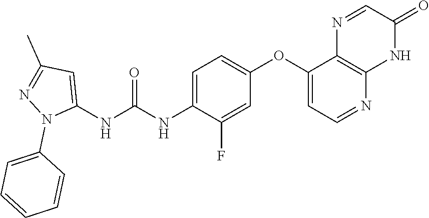 [SMILES: Cc1cc(NC(=O)Nc2ccc(Oc3ccnc4[nH]c(=O)cnc34)cc2F)n(-c2ccccc2)n1] | 1-(2-fluoro-4-((3-oxo-3,4- dihydropyrido[2,3-b]pyrazin-8- yl)oxy)phenyl)-3-(3-methyl-1-phenyl-1H- pyrazol-5-yl)urea |
| (b) | 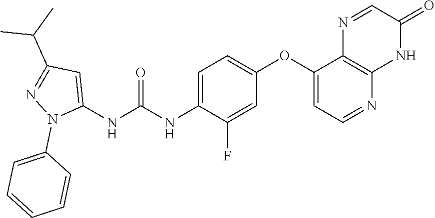 [SMILES: CC(C)c1cc(NC(=O)Nc2ccc(Oc3ccnc4[nH]c(=O)cnc34)cc2F)n(-c2ccccc2)n1] | 1-(2-fluoro-4-((3-oxo-3,4- dihydropyrido[2,3-b]pyrazin-8- yl)oxy)phenyl)-3-(3-isopropyl-1-phenyl- 1H-pyrazol-5-yl)urea |
| (c) | 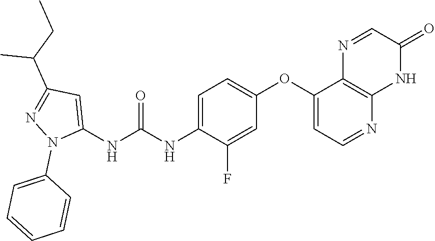 [SMILES: CCC(C)c1cc(NC(=O)Nc2ccc(Oc3ccnc4[nH]c(=O)cnc34)cc2F)n(-c2ccccc2)n1] | 1-(3-(sec-butyl)-1-phenyl-1H-pyrazol-5- yl)-3-(2-fluoro-4-((3-oxo-3,4- dihydropyrido[2,3-b]pyrazin-8- yl)oxy)phenyl)urea |
| (d) | 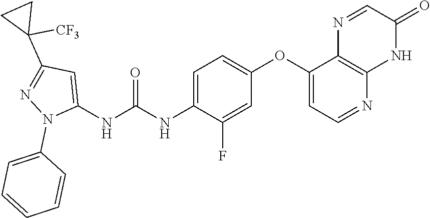 [SMILES: O=C(Nc1ccc(Oc2ccnc3[nH]c(=O)cnc23)cc1F)Nc1cc(C2(C(F)(F)F)CC2)nn1-c1ccccc1] | 1-(2-fluoro-4-((3-oxo-3,4- dihydropyrido[2,3-b]pyrazin-8- yl)oxy)phenyl)-3-(1-phenyl-3-(1- (trifluoromethyl)cyclopropyl)-1H-pyrazol- 5-yl)urea |
| (e) | 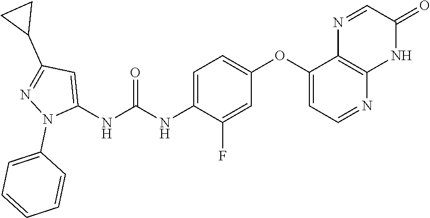 [SMILES: O=C(Nc1ccc(Oc2ccnc3[nH]c(=O)cnc23)cc1F)Nc1cc(C2CC2)nn1-c1ccccc1] | 1-(3-cyclopropyl-1-phenyl-1H-pyrazol-5- yl)-3-(2-fluoro-4-((3-oxo-3,4- dihydropyrido[2,3-b]pyrazin-8- yl)oxy)phenyl)urea |
| (f) | 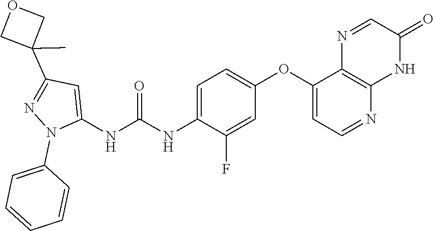 [SMILES: CC1(c2cc(NC(=O)Nc3ccc(Oc4ccnc5[nH]c(=O)cnc45)cc3F)n(-c3ccccc3)n2)COC1] | 1-(2-fluoro-4-((3-oxo-3,4- dihydropyrido[2,3-b]pyrazin-8- yl)oxy)phenyl)-3-(3-(3-methyloxetan-3- yl)-1-phenyl-1H-pyrazol-5-yl)urea |
| (g) | 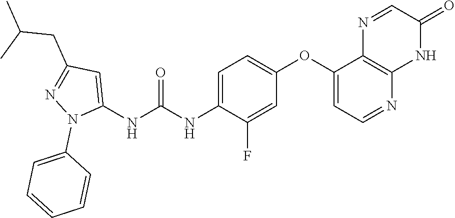 [SMILES: CC(C)Cc1cc(NC(=O)Nc2ccc(Oc3ccnc4[nH]c(=O)cnc34)cc2F)n(-c2ccccc2)n1] | 1-(2-fluoro-4-((3-oxo-3,4- dihydropyrido[2,3-b]pyrazin-8- yl)oxy)phenyl)-3-(3-isobutyl-1-phenyl- 1H-pyrazol-5-yl)urea |
| (h) | 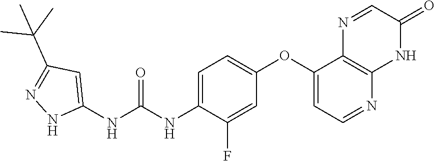 [SMILES: CC(C)(C)c1cc(NC(=O)Nc2ccc(Oc3ccnc4[nH]c(=O)cnc34)cc2F)[nH]n1] | 1-(3-(tert-butyl)-1H-pyrazol-5-yl)-3-(2- fluoro-4-((3-oxo-3,4-dihydropyrido[2,3- b]pyrazin-8-yl)oxy)phenyl)urea |
| (i) | 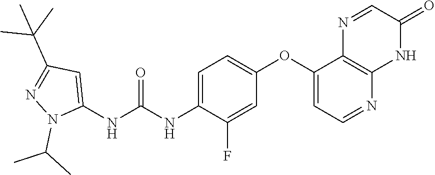 [SMILES: CC(C)n1nc(C(C)(C)C)cc1NC(=O)Nc1ccc(Oc2ccnc3[nH]c(=O)cnc23)cc1F] | 1-(3-(tert-butyl)-1-isopropyl-1H-pyrazol- 5-yl)-3-(2-fluoro-4-((3-oxo-3,4- dihydropyrido[2,3-b]pyrazin-8- yl)oxy)phenyl)urea |
| (j) | 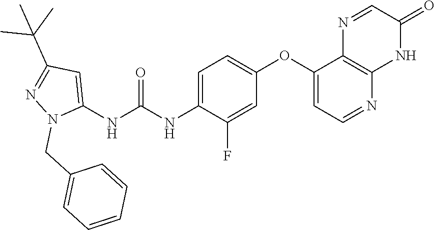 [SMILES: CC(C)(C)c1cc(NC(=O)Nc2ccc(Oc3ccnc4[nH]c(=O)cnc34)cc2F)n(Cc2ccccc2)n1] | 1-(1-benzyl-3-(tert-butyl)-1H-pyrazol-5- yl)-3-(2-fluoro-4-((3-oxo-3,4- dihydropyrido[2,3-b]pyrazin-8- yl)oxy)phenyl)urea |
| (k) | 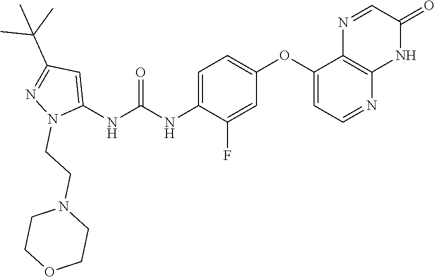 [SMILES: CC(C)(C)c1cc(NC(=O)Nc2ccc(Oc3ccnc4[nH]c(=O)cnc34)cc2F)n(CCN2CCOCC2)n1] | 1-(3-(tert-butyl)-1-(2-morpholinoethyl)- 1H-pyrazol-5-yl)-3-(2-fluoro-4-((3-oxo- 3,4-dihydropyrido[2,3-b]pyrazin-8- yl)oxy)phenyl)urea |
| (l) | 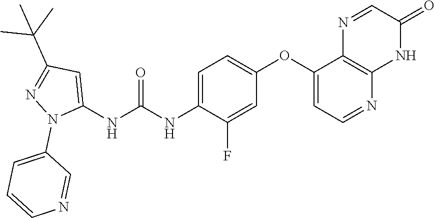 [SMILES: CC(C)(C)c1cc(NC(=O)Nc2ccc(Oc3ccnc4[nH]c(=O)cnc34)cc2F)n(-c2cccnc2)n1] | 1-(3-(tert-butyl)-1-(pyridin-3-yl)-1H- pyrazol-5-yl)-3-(2-fluoro-4-((3-oxo-3,4- dihydropyrido[2,3-b]pyrazin-8- yl)oxy)phenyl)urea |
| (m) | 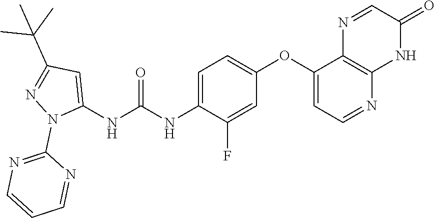 [SMILES: CC(C)(C)c1cc(NC(=O)Nc2ccc(Oc3ccnc4[nH]c(=O)cnc34)cc2F)n(-c2ncccn2)n1] | 1-(3-(tert-butyl)-1-(pyrimidin-2-yl)-1H- pyrazol-5-yl)-3-(2-fluoro-4-((3-oxo-3,4- dihydropyrido[2,3-b]pyrazin-8- yl)oxy)phenyl)urea |
| (n) | 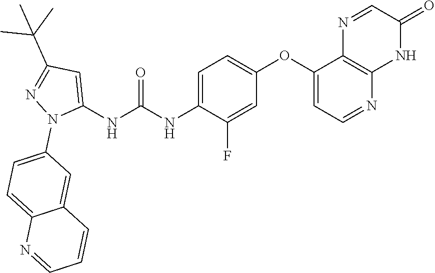 [SMILES: CC(C)(C)c1cc(NC(=O)Nc2ccc(Oc3ccnc4[nH]c(=O)cnc34)cc2F)n(-c2ccc3ncccc3c2)n1] | 1-(3-(tert-butyl)-1-(quinolin-6-yl)-1H- pyrazol-5-yl)-3-(2-fluoro-4-((3-oxo-3,4- dihydropyrido[2,3-b]pyrazin-8- yl)oxy)phenyl)urea |
| (o) | 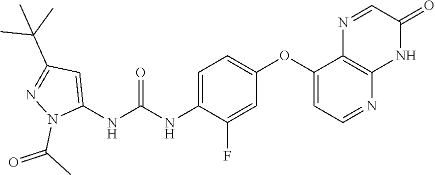 [SMILES: CC(=O)n1nc(C(C)(C)C)cc1NC(=O)Nc1ccc(Oc2ccnc3[nH]c(=O)cnc23)cc1F] | 1-(1-acetyl-3-(tert-butyl)-1H-pyrazol-5- yl)-3-(2-fluoro-4-((3-oxo-3,4- dihydropyrido[2,3-b]pyrazin-8- yl)oxy)phenyl)urea |
| (p) | 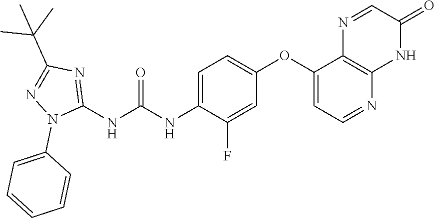 [SMILES: CC(C)(C)c1nc(NC(=O)Nc2ccc(Oc3ccnc4[nH]c(=O)cnc34)cc2F)n(-c2ccccc2)n1] | 1-(3-(tert-butyl)-1-phenyl-1H-1,2,4- triazol-5-yl)-3-(2-fluoro-4-((3-oxo-3,4- dihydropyrido[2,3-b]pyrazin-8- yl)oxy)phenyl)urea |
| (q) | 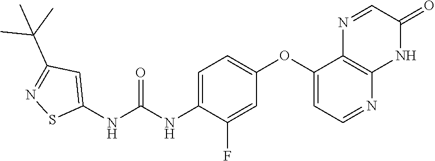 [SMILES: CC(C)(C)c1cc(NC(=O)Nc2ccc(Oc3ccnc4[nH]c(=O)cnc34)cc2F)sn1] | 1-(3-(tert-butyl)isothiazol-5-yl)-3-(2- fluoro-4-((3-oxo-3,4-dihydropyrido[2,3- b]pyrazin-8-yl)oxy)phenyl)urea |
| (r) | 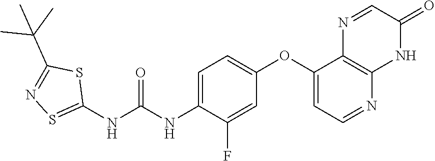 | 1-(5-(tert-butyl)-1,3,4-thiadiazol-2-yl)-3- (2-fluoro-4-((3-oxo-3,4- dihydropyrido[2,3-b]pyrazin-8- yl)oxy)phenyl)urea |
| (s) | 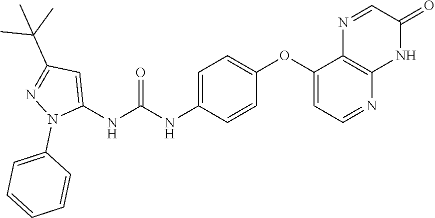 [SMILES: CC(C)(C)c1cc(NC(=O)Nc2ccc(Oc3ccnc4[nH]c(=O)cnc34)cc2)n(-c2ccccc2)n1] | 1-(3-(tert-butyl)-1-phenyl-1H-pyrazol-5- yl)-3-(4-((3-oxo-3,4-dihydropyrido[2,3- b]pyrazin-8-yl)oxy)phenyl)urea |
| (t) | 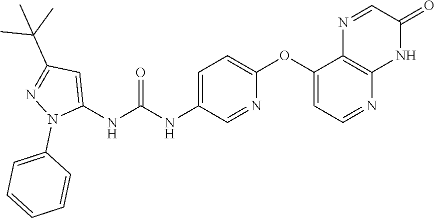 [SMILES: CC(C)(C)c1cc(NC(=O)Nc2ccc(Oc3ccnc4[nH]c(=O)cnc34)nc2)n(-c2ccccc2)n1] | 1-(3-(tert-butyl)-1-phenyl-1H-pyrazol-5- yl)-3-(6-((3-oxo-3,4-dihydropyrido[2,3- b]pyrazin-8-yl)oxy)pyridin-3-yl)urea |
| (u) | 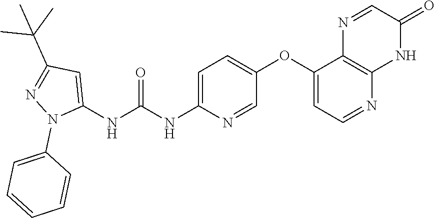 [SMILES: CC(C)(C)c1cc(NC(=O)Nc2ccc(Oc3ccnc4[nH]c(=O)cnc34)cn2)n(-c2ccccc2)n1] | 1-(3-(tert-butyl)-1-phenyl-1H-pyrazol-5- yl)-3-(5-((3-oxo-3,4-dihydropyrido[2,3- b]pyrazin-8-yl)oxy)pyridin-2-yl)urea |
| (v) | 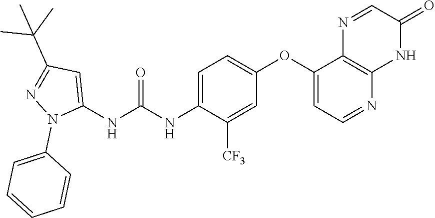 [SMILES: CC(C)(C)c1cc(NC(=O)Nc2ccc(Oc3ccnc4[nH]c(=O)cnc34)cc2C(F)(F)F)n(-c2ccccc2)n1] | 1-(3-(tert-butyl)-1-phenyl-1H-pyrazol-5- yl)-3-(4-((3-oxo-3,4-dihydropyrido[2,3- b]pyrazin-8-yl)oxy)-2- (trifluoromethyl)phenyl)urea |
| (w) | 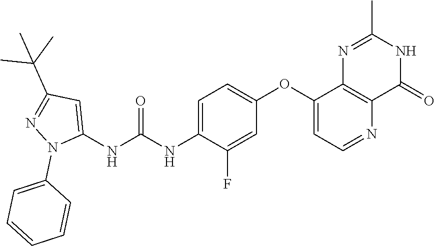 [SMILES: Cc1nc2c(Oc3ccc(NC(=O)Nc4cc(C(C)(C)C)nn4-c4ccccc4)c(F)c3)ccnc2c(=O)[nH]1] | 1-(3-(tert-butyl)-1-phenyl-1H-pyrazol-5- yl)-3-(2-fluoro-4-((2-methyl-4-oxo-3,4- dihydropyrido[3,2-d]pyrimidin-8- yl)oxy)phenyl)urea |
| (x) | 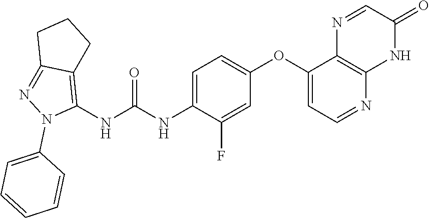 [SMILES: O=C(Nc1ccc(Oc2ccnc3[nH]c(=O)cnc23)cc1F)Nc1c2c(nn1-c1ccccc1)CCC2] | 1-(2-fluoro-4-((3-oxo-3,4- dihydropyrido[2,3-b]pyrazin-8- yl)oxy)phenyl)-3-(2-phenyl-2,4,5,6- tetrahydrocyclopenta[c]pyrazol-3- yl)urea |
| (y) | 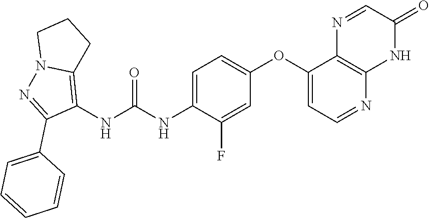 [SMILES: O=C(Nc1ccc(Oc2ccnc3[nH]c(=O)cnc23)cc1F)Nc1c(-c2ccccc2)nn2c1CCC2] | 1-(2-fluoro-4-((3-oxo-3,4- dihydropyrido[2,3-b]pyrazin-8- yl)oxy)phenyl)-3-(2-phenyl-5,6-dihydro- 4H-pyrrolo[1,2-b]pyrazol-3-yl)urea |
| (z) | 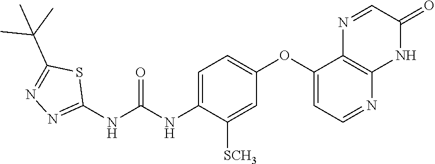 [SMILES: CSc1cc(Oc2ccnc3[nH]c(=O)cnc23)ccc1NC(=O)Nc1nnc(C(C)(C)C)s1] | 1-(5-(tert-butyl)-1,3,4-thiadiazol-2-yl)-3- (2-(methylthio)-4-((3-oxo-3,4- dihydropyrido[2,3-b]pyrazin-8- yl)oxy)phenyl)urea |
| (aa) | 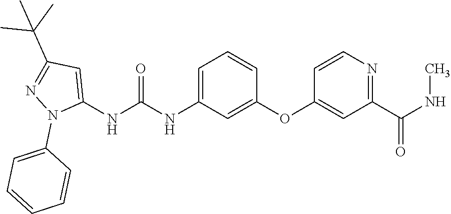 [SMILES: CNC(=O)c1cc(Oc2cccc(NC(=O)Nc3cc(C(C)(C)C)nn3-c3ccccc3)c2)ccn1] | 4-(3-(3-(3-(tert-butyl)-1-phenyl-1H- pyrazol-5-yl)ureido)phenoxy)-N- methylpicolinamide |
| (ab) | 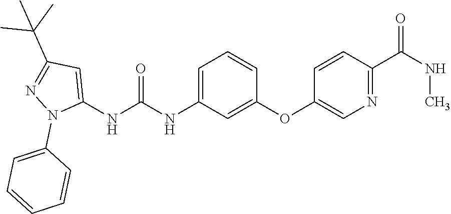 [SMILES: CNC(=O)c1ccc(Oc2cccc(NC(=O)Nc3cc(C(C)(C)C)nn3-c3ccccc3)c2)cn1] | 5-(3-(3-(3-(tert-butyl)-1-phenyl-1H- pyrazol-5-yl)ureido)phenoxy)-N- methylpicolinamide |
| (ac) | 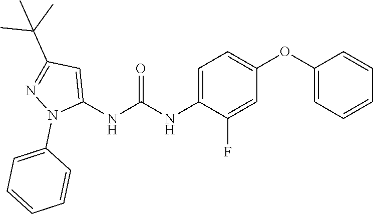 [SMILES: CC(C)(C)c1cc(NC(=O)Nc2ccc(Oc3ccccc3)cc2F)n(-c2ccccc2)n1] | 1-(3-(tert-butyl)-1-phenyl-1H-pyrazol-5- yl)-3-(2-fluoro-4-phenoxyphenyl)urea |
| (ad) | 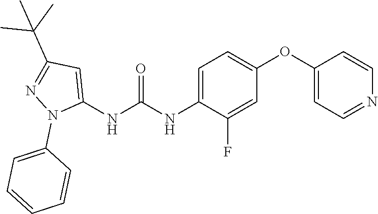 [SMILES: CC(C)(C)c1cc(NC(=O)Nc2ccc(Oc3ccncc3)cc2F)n(-c2ccccc2)n1] | 1-(3-(tert-butyl)-1-phenyl-1H-pyrazol-5- yl)-3-(2-fluoro-4-(pyridin-4- yloxy)phenyl)urea |
| (ae) | 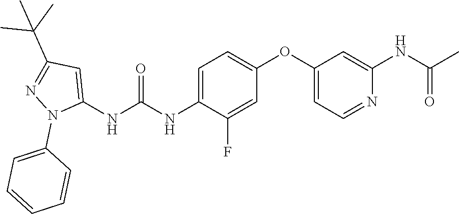 [SMILES: CC(=O)Nc1cc(Oc2ccc(NC(=O)Nc3cc(C(C)(C)C)nn3-c3ccccc3)c(F)c2)ccn1] | N-(4-(4-(3-(3-(tert-butyl)-1-phenyl-1H- pyrazol-5-yl)ureido)-3- fluorophenoxy)pyridin-2-yl)acetamide |
| (af) | 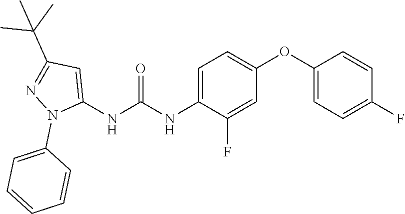 [SMILES: CC(C)(C)c1cc(NC(=O)Nc2ccc(Oc3ccc(F)cc3)cc2F)n(-c2ccccc2)n1] | 1-(3-(tert-butyl)-1-phenyl-1H-pyrazol-5- yl)-3-(2-fluoro-4-(4- fluorophenoxy)phenyl)urea |
| (ag) | 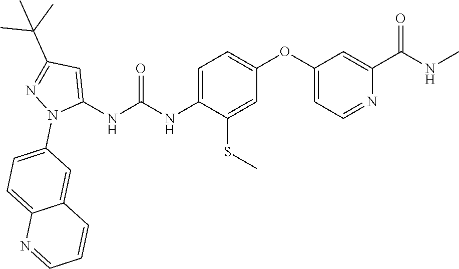 [SMILES: CNC(=O)c1cc(Oc2ccc(NC(=O)Nc3cc(C(C)(C)C)nn3-c3ccc4ncccc4c3)c(SC)c2)ccn1] | 4-(4-(3-(3-(tert-butyl)-1-(quinolin-6-yl)- 1H-pyrazol-5-yl)ureido)-3- (methylthio)phenoxy)-N- methylpicolinamide |
| (ah) | 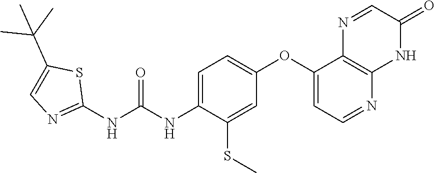 [SMILES: CSc1cc(Oc2ccnc3[nH]c(=O)cnc23)ccc1NC(=O)Nc1ncc(C(C)(C)C)s1] | 1-(5-(tert-butyl)thiazol-2-yl)-3-(2- (methylthio)-4-((3-oxo-3,4- dihydropyrido[2,3-b]pyrazin-8- yl)oxy)phenyl)urea |
| (ai) | 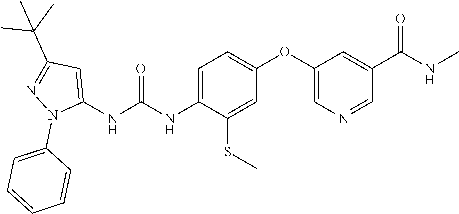 [SMILES: CNC(=O)c1cncc(Oc2ccc(NC(=O)Nc3cc(C(C)(C)C)nn3-c3ccccc3)c(SC)c2)c1] | 5-(4-(3-(3-(tert-butyl)-1-phenyl-1H- pyrazol-5-yl)ureido)-3- (methylthio)phenoxy)-N- methylnicotinamide |
| (aj) | 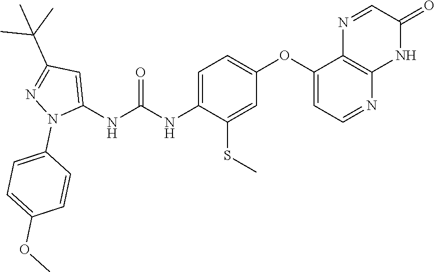 [SMILES: COc1ccc(-n2nc(C(C)(C)C)cc2NC(=O)Nc2ccc(Oc3ccnc4[nH]c(=O)cnc34)cc2SC)cc1] | 1-(3-(tert-butyl)-1-(4-methoxyphenyl)- 1H-pyrazol-5-yl)-3-(2-(methylthio)-4-((3- oxo-3,4-dihydropyrido[2,3-b]pyrazin-8- yl)oxy)phenyl)urea |
| (ak) | 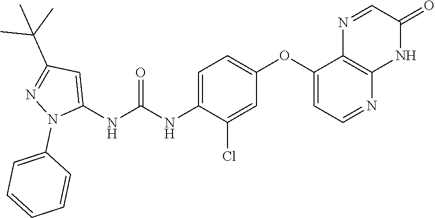 [SMILES: CC(C)(C)c1cc(NC(=O)Nc2ccc(Oc3ccnc4[nH]c(=O)cnc34)cc2Cl)n(-c2ccccc2)n1] | 1-(3-(tert-butyl)-1-phenyl-1H-pyrazol-5- yl)-3-(2-chloro-4-((3-oxo-3,4- dihydropyrido[2,3-b]pyrazin-8- yl)oxy)phenyl)urea |
| (al) | 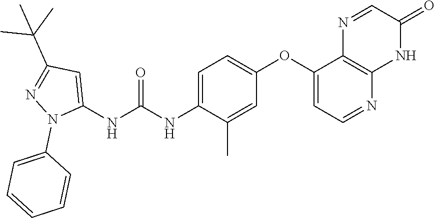 [SMILES: Cc1cc(Oc2ccnc3[nH]c(=O)cnc23)ccc1NC(=O)Nc1cc(C(C)(C)C)nn1-c1ccccc1] | 1-(3-(tert-butyl)-1-phenyl-1H-pyrazol-5- yl)-3-(2-methyl-4-((3-oxo-3,4- dihydropyrido[2,3-b]pyrazin-8- yl)oxy)phenyl)urea |
| (am) | 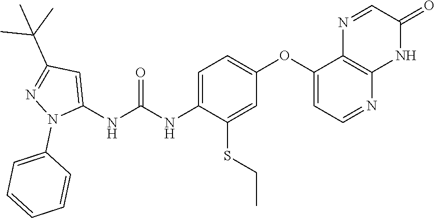 [SMILES: CCSc1cc(Oc2ccnc3[nH]c(=O)cnc23)ccc1NC(=O)Nc1cc(C(C)(C)C)nn1-c1ccccc1] | 1-(3-(tert-butyl)-1-phenyl-1H-pyrazol-5- yl)-3-(2-(ethylthio)-4-((3-oxo-3,4- dihydropyrido[2,3-b]pyrazin-8- yl)oxy)phenyl)urea |
| (an) | 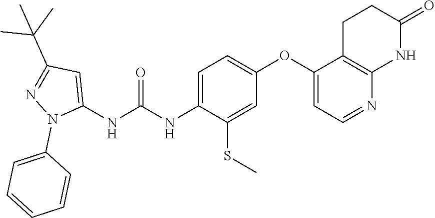 [SMILES: CSc1cc(Oc2ccnc3c2CCC(=O)N3)ccc1NC(=O)Nc1cc(C(C)(C)C)nn1-c1ccccc1] | 1-(3-(tert-butyl)-1-phenyl-1H-pyrazol-5- yl)-3-(2-(methylthio)-4-((7-oxo-5,6,7,8- tetrahydro-1,8-naphthyridin-4- yl)oxy)phenyl)urea |
| (ao) | 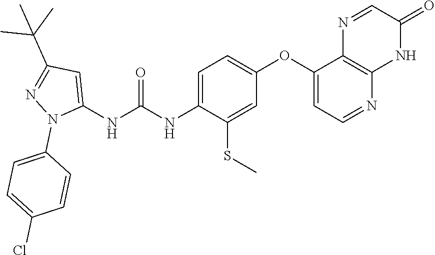 [SMILES: CSc1cc(Oc2ccnc3[nH]c(=O)cnc23)ccc1NC(=O)Nc1cc(C(C)(C)C)nn1-c1ccc(Cl)cc1] | 1-(3-(tert-butyl)-1-(4-chlorophenyl)-1H- pyrazol-5-yl)-3-(2-(methylthio)-4-((3- oxo-3,4-dihydropyrido[2,3-b]pyrazin-8- yl)oxy)phenyl)urea |
| (ap) | 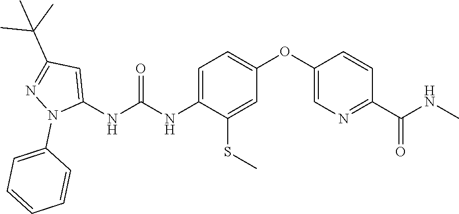 [SMILES: CNC(=O)c1ccc(Oc2ccc(NC(=O)Nc3cc(C(C)(C)C)nn3-c3ccccc3)c(SC)c2)cn1] | 5-(4-(3-(3-(tert-butyl)-1-phenyl-1H- pyrazol-5-yl)ureido)-3- (methylthio)phenoxy)-N- methylpicolinamide |
| (aq) | 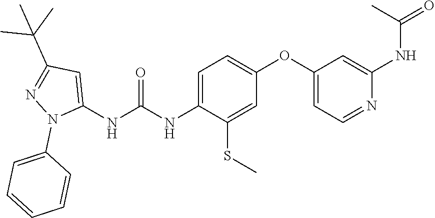 [SMILES: CSc1cc(Oc2ccnc(NC(C)=O)c2)ccc1NC(=O)Nc1cc(C(C)(C)C)nn1-c1ccccc1] | N-(4-(4-(3-(3-(tert-butyl)-1-phenyl-1H- pyrazol-5-yl)ureido)-3- (methylthio)phenoxy)pyridin-2- yl)acetamide |
| (ar) | 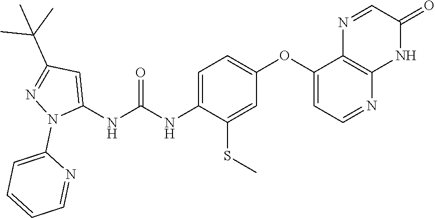 [SMILES: CSc1cc(Oc2ccnc3[nH]c(=O)cnc23)ccc1NC(=O)Nc1cc(C(C)(C)C)nn1-c1ccccn1] | 1-(3-(tert-butyl)-1-(pyridin-2-yl)-1H- pyrazol-5-yl)-3-(2-(methylthio)-4-((3- oxo-3,4-dihydropyrido[2,3-b]pyrazin-8- yl)oxy)phenyl)urea |
| (as) | 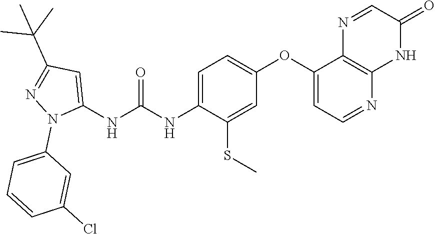 [SMILES: CSc1cc(Oc2ccnc3[nH]c(=O)cnc23)ccc1NC(=O)Nc1cc(C(C)(C)C)nn1-c1cccc(Cl)c1] | 1-(3-(tert-butyl)-1-(3-chlorophenyl)-1H- pyrazol-5-yl)-3-(2-(methylthio)-4-((3- oxo-3,4-dihydropyrido[2,3-b]pyrazin-8- yl)oxy)phenyl)urea |
| (at) | 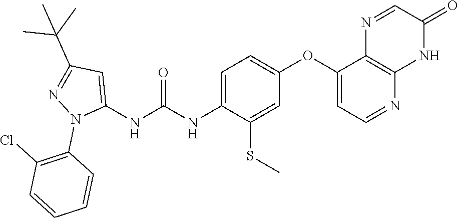 [SMILES: CSc1cc(Oc2ccnc3[nH]c(=O)cnc23)ccc1NC(=O)Nc1cc(C(C)(C)C)nn1-c1ccccc1Cl] | 1-(3-(tert-butyl)-1-(2-chlorophenyl)-1H- pyrazol-5-yl)-3-(2-(methylthio)-4-((3- oxo-3,4-dihydropyrido[2,3-b]pyrazin-8- yl)oxy)phenyl)urea |
| (au) | 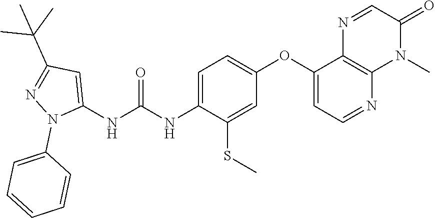 [SMILES: CSc1cc(Oc2ccnc3c2ncc(=O)n3C)ccc1NC(=O)Nc1cc(C(C)(C)C)nn1-c1ccccc1] | 1-(3-(tert-butyl)-1-phenyl-1H-pyrazol-5- yl)-3-(4-((4-methyl-3-oxo-3,4- dihydropyrido[2,3-b]pyrazin-8-yl)oxy)-2- (methylthio)phenyl)urea |
| (av) | 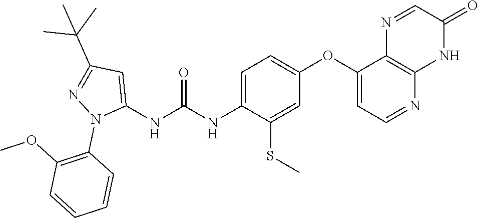 [SMILES: COc1ccccc1-n1nc(C(C)(C)C)cc1NC(=O)Nc1ccc(Oc2ccnc3[nH]c(=O)cnc23)cc1SC] | 1-(3-(tert-butyl)-1-(2-methoxyphenyl)- 1H-pyrazol-5-yl)-3-(2-(methylthio)-4-((3- oxo-3,4-dihydropyrido[2,3-b]pyrazin-8- yl)oxy)phenyl)urea |
| (aw) | 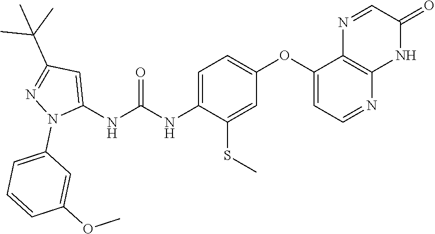 [SMILES: COc1cccc(-n2nc(C(C)(C)C)cc2NC(=O)Nc2ccc(Oc3ccnc4[nH]c(=O)cnc34)cc2SC)c1] | 1-(3-(tert-butyl)-1-(3-methoxyphenyl)- 1H-pyrazol-5-yl)-3-(2-(methylthio)-4-((3- oxo-3,4-dihydropyrido[2,3-b]pyrazin-8- yl)oxy)phenyl)urea |
| (ax) | 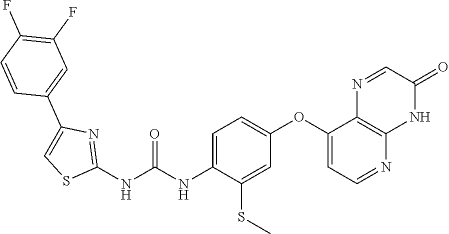 [SMILES: CSc1cc(Oc2ccnc3[nH]c(=O)cnc23)ccc1NC(=O)Nc1nc(-c2ccc(F)c(F)c2)cs1] | 1-(4-(3,4-difluorophenyl)thiazol-2-yl)-3- (2-(methylthio)-4-((3-oxo-3,4- dihydropyrido[2,3-b]pyrazin-8- yl)oxy)phenyl)urea |
| (ay) | 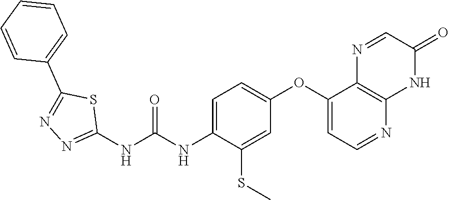 [SMILES: CSc1cc(Oc2ccnc3[nH]c(=O)cnc23)ccc1NC(=O)Nc1nnc(-c2ccccc2)s1] | 1-(2-(methylthio)-4-((3-oxo-3,4- dihydropyrido[2,3-b]pyrazin-8- yl)oxy)phenyl)-3-(5-phenyl-1,3,4- thiadiazol-2-yl)urea |
| (az) | 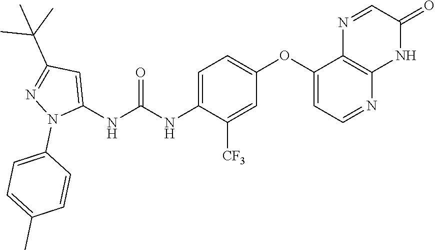 [SMILES: Cc1ccc(-n2nc(C(C)(C)C)cc2NC(=O)Nc2ccc(Oc3ccnc4[nH]c(=O)cnc34)cc2C(F)(F)F)cc1] | 1-(3-(tert-butyl)-1-(p-tolyl)-1H-pyrazol-5- yl)-3-(4-((3-oxo-3,4-dihydropyrido[2,3- b]pyrazin-8-yl)oxy)-2- (trifluoromethyl)phenyl)urea |
| (ba) | 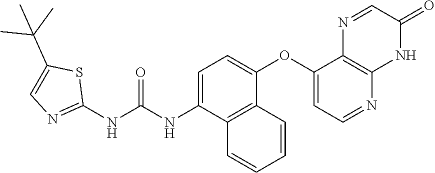 [SMILES: CC(C)(C)c1cnc(NC(=O)Nc2ccc(Oc3ccnc4[nH]c(=O)cnc34)c3ccccc23)s1] | 1-(5-(tert-butyl)thiazol-2-yl)-3-(4-((3-oxo- 3,4-dihydropyrido[2,3-b]pyrazin-8- yl)oxy)naphthalen-1-yl)urea |
| (bb) | 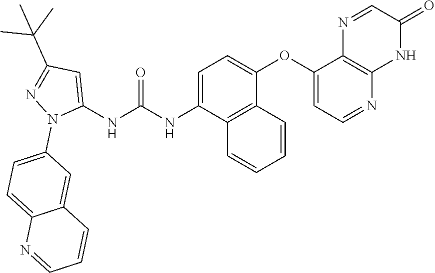 [SMILES: CC(C)(C)c1cc(NC(=O)Nc2ccc(Oc3ccnc4[nH]c(=O)cnc34)c3ccccc23)n(-c2ccc3ncccc3c2)n1] | 1-(3-(tert-butyl)-1-(quinolin-6-yl)-1H- pyrazol-5-yl)-3-(4-((3-oxo-3,4- dihydropyrido[2,3-b]pyrazin-8- yl)oxy)naphthalen-1-yl)urea |
| (bc) | 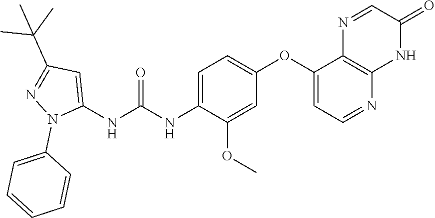 [SMILES: COc1cc(Oc2ccnc3[nH]c(=O)cnc23)ccc1NC(=O)Nc1cc(C(C)(C)C)nn1-c1ccccc1] | 1-(3-(tert-butyl)-1-phenyl-1H-pyrazol-5- yl)-3-(2-methoxy-4-((3-oxo-3,4- dihydropyrido[2,3-b]pyrazin-8- yl)oxy)phenyl)urea |
| (bd) | 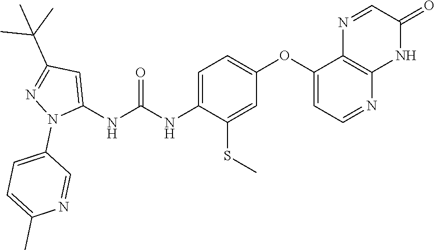 [SMILES: CSc1cc(Oc2ccnc3[nH]c(=O)cnc23)ccc1NC(=O)Nc1cc(C(C)(C)C)nn1-c1ccc(C)nc1] | 1-(3-(tert-butyl)-1-(6-methylpyridin-3-yl)- 1H-pyrazol-5-yl)-3-(2-(methylthio)-4-((3- oxo-3,4-dihydropyrido[2,3-b]pyrazin-8- yl)oxy)phenyl)urea |
| (be) | 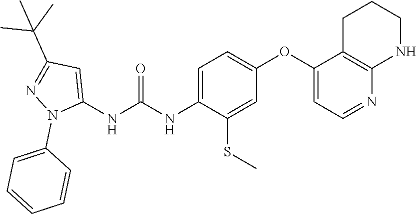 [SMILES: CSc1cc(Oc2ccnc3c2CCCN3)ccc1NC(=O)Nc1cc(C(C)(C)C)nn1-c1ccccc1] | 1-(3-(tert-butyl)-1-phenyl-1H-pyrazol-5- yl)-3-(2-(methylthio)-4-((5,6,7,8- tetrahydro-1,8-naphthyridin-4- yl)oxy)phenyl)urea |
| (bf) | 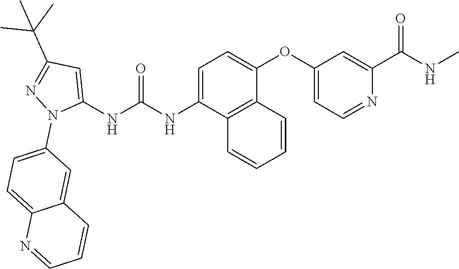 [SMILES: CNC(=O)c1cc(Oc2ccc(NC(=O)Nc3cc(C(C)(C)C)nn3-c3ccc4ncccc4c3)c3ccccc23)ccn1] | 4-((4-(3-(3-(tert-butyl)-1-(quinolin-6-yl)- 1H-pyrazol-5-yl)ureido)naphthalen-1- yl)oxy)-N-methylpicolinamide |
| (bg) | 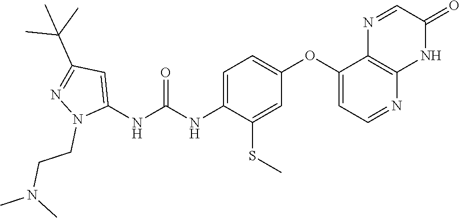 [SMILES: CSc1cc(Oc2ccnc3[nH]c(=O)cnc23)ccc1NC(=O)Nc1cc(C(C)(C)C)nn1CCN(C)C] | 1-(3-(tert-butyl)-1-(2- (dimethylamino)ethyl)-1H-pyrazol-5-yl)- 3-(2-(methylthio)-4-((3-oxo-3,4- dihydropyrido[2,3-b]pyrazin-8- yl)oxy)phenyl)urea |
| (bh) | 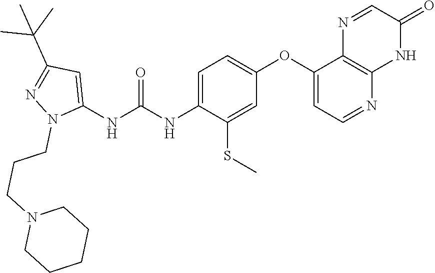 [SMILES: CSc1cc(Oc2ccnc3[nH]c(=O)cnc23)ccc1NC(=O)Nc1cc(C(C)(C)C)nn1CCCN1CCCCC1] | 1-(3-(tert-butyl)-1-(3-morpholinopropyl)- 1H-pyrazol-5-yl)-3-(2-(methylthio)-4-((3- oxo-3,4-dihydropyrido[2,3-b]pyrazin-8- yl)oxy)phenyl)urea |
| (bi) | 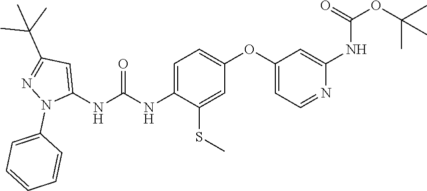 [SMILES: CSc1cc(Oc2ccnc(NC(=O)OC(C)(C)C)c2)ccc1NC(=O)Nc1cc(C(C)(C)C)nn1-c1ccccc1] | tert-butyl (4-(4-(3-(3-(tert-butyl)-1- phenyl-1H-pyrazol-5-yl)ureido)-3- (methylthio)phenoxy)pyridin-2- yl)carbamate |
| (bj) | 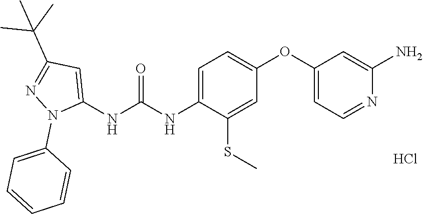 [SMILES: CSc1cc(Oc2ccnc(N)c2)ccc1NC(=O)Nc1cc(C(C)(C)C)nn1-c1ccccc1 ; Cl] | 1-(4-((2-aminopyridin-4-yl)oxy)-2- (methylthio)phenyl)-3-(3-(tert-butyl)-1- phenyl-1H-pyrazol-5-yl)urea hydrochloride |
| (bk) | 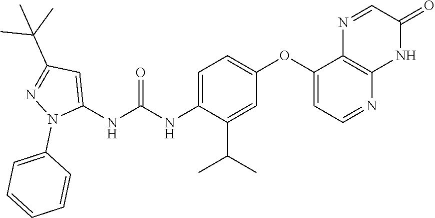 [SMILES: CC(C)c1cc(Oc2ccnc3[nH]c(=O)cnc23)ccc1NC(=O)Nc1cc(C(C)(C)C)nn1-c1ccccc1] | 1-(3-(tert-butyl)-1-phenyl-1H-pyrazol-5- yl)-3-(2-isopropyl-4-((3-oxo-3,4- dihydropyrido[2,3-b]pyrazin-8- yl)oxy)phenyl)urea |
| (bl) | 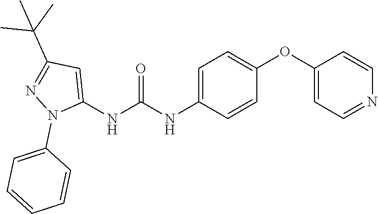 [SMILES: CC(C)(C)c1cc(NC(=O)Nc2ccc(Oc3ccncc3)cc2)n(-c2ccccc2)n1] | 1-(3-(tert-butyl)-1-phenyl-1H-pyrazol-5- yl)-3-(4-(pyridin-4-yloxy)phenyl)urea |
| (bm) | 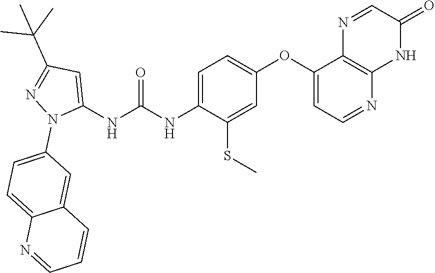 [SMILES: CSc1cc(Oc2ccnc3[nH]c(=O)cnc23)ccc1NC(=O)Nc1cc(C(C)(C)C)nn1-c1ccc2ncccc2c1] | 1-(3-(tert-butyl)-1-(quinolin-6-yl)-1H- pyrazol-5-yl)-3-(2-(methylthio)-4-((3- oxo-3,4-dihydropyrido[2,3-b]pyrazin-8- yl)oxy)phenyl)urea |
| (bn) | 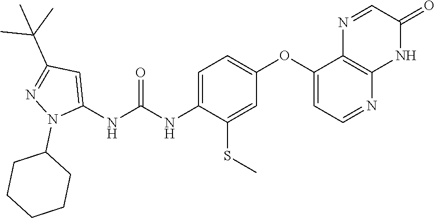 [SMILES: CSc1cc(Oc2ccnc3[nH]c(=O)cnc23)ccc1NC(=O)Nc1cc(C(C)(C)C)nn1C1CCCCC1] | 1-(3-(tert-butyl)-1-cyclohexyl-1H- pyrazol-5-yl)-3-(2-(methylthio)-4-((3- oxo-3,4-dihydropyrido[2,3-b]pyrazin-8- yl)oxy)phenyl)urea |
| (bo) | 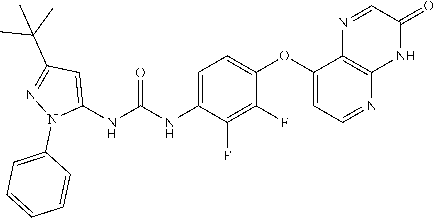 [SMILES: CC(C)(C)c1cc(NC(=O)Nc2ccc(Oc3ccnc4[nH]c(=O)cnc34)c(F)c2F)n(-c2ccccc2)n1] | 1-(3-(tert-butyl)-1-phenyl-1H-pyrazol-5- yl)-3-(2,3-difluoro-4-((3-oxo-3,4- dihydropyrido[2,3-b]pyrazin-8- yl)oxy)phenyl)urea |
| (bp) | 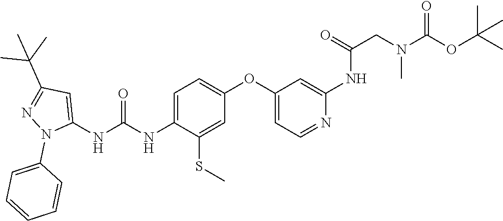 [SMILES: CSc1cc(Oc2ccnc(NC(=O)CN(C)C(=O)OC(C)(C)C)c2)ccc1NC(=O)Nc1cc(C(C)(C)C)nn1-c1ccccc1] | tert-butyl (2-((4-(4-(3-(3-(tert-butyl)-1- phenyl-1H-pyrazol-5-yl)ureido)-3- (methylthio)phenoxy)pyridin-2- yl)amino)-2-oxoethyl)(methyl)carbamate |
| (bq) | 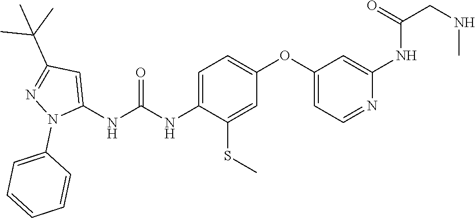 [SMILES: CNCC(=O)Nc1cc(Oc2ccc(NC(=O)Nc3cc(C(C)(C)C)nn3-c3ccccc3)c(SC)c2)ccn1] | N-(4-(4-(3-(3-(tert-butyl)-1-phenyl-1H- pyrazol-5-yl)ureido)-3- (methylthio)phenoxy)pyridin-2-yl)-2- (methylamino)acetamide |
| (br) | 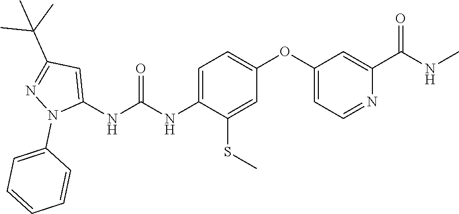 [SMILES: CNC(=O)c1cc(Oc2ccc(NC(=O)Nc3cc(C(C)(C)C)nn3-c3ccccc3)c(SC)c2)ccn1] | 4-(4-(3-(3-(tert-butyl)-1-phenyl-1H- pyrazol-5-yl)ureido)-3- (methylthio)phenoxy)-N- methylpicolinamide |
| (bs) | 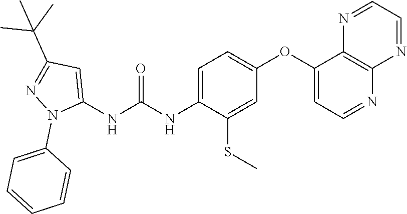 [SMILES: CSc1cc(Oc2ccnc3nccnc23)ccc1NC(=O)Nc1cc(C(C)(C)C)nn1-c1ccccc1] | 1-(3-(tert-butyl)-1-phenyl-1H-pyrazol-5- yl)-3-(2-(methylthio)-4-(pyrido[2,3- b]pyrazin-8-yloxy)phenyl)urea |
| (bt) | 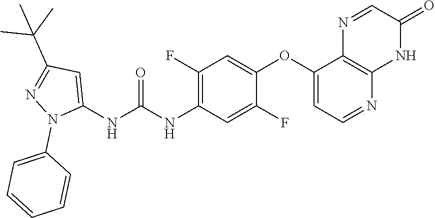 [SMILES: CC(C)(C)c1cc(NC(=O)Nc2cc(F)c(Oc3ccnc4[nH]c(=O)cnc34)cc2F)n(-c2ccccc2)n1] | 1-(3-(tert-butyl)-1-phenyl-1H-pyrazol-5- yl)-3-(2,5-difluoro-4-((3-oxo-3,4- dihydropyrido[2,3-b]pyrazin-8- yl)oxy)phenyl)urea |
| (bu) | 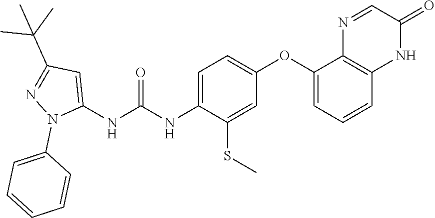 [SMILES: CSc1cc(Oc2cccc3[nH]c(=O)cnc23)ccc1NC(=O)Nc1cc(C(C)(C)C)nn1-c1ccccc1] | 1-(3-(tert-butyl)-1-phenyl-1H-pyrazol-5- yl)-3-(2-(methylthio)-4-((2-oxo-1,2- dihydroquinoxalin-5-yl)oxy)phenyl)urea |
| (bv) | 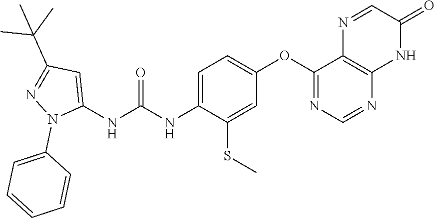 [SMILES: CSc1cc(Oc2ncnc3[nH]c(=O)cnc23)ccc1NC(=O)Nc1cc(C(C)(C)C)nn1-c1ccccc1] | 1-(3-(tert-butyl)-1-phenyl-1H-pyrazol-5- yl)-3-(2-(methylthio)-4-((7-oxo-7,8- dihydropteridin-4-yl)oxy)phenyl)urea |
| (bw) | 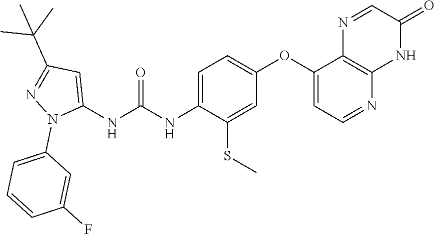 [SMILES: CSc1cc(Oc2ccnc3[nH]c(=O)cnc23)ccc1NC(=O)Nc1cc(C(C)(C)C)nn1-c1cccc(F)c1] | 1-(3-(tert-butyl)-1-(3-fluorophenyl)-1H- pyrazol-5-yl)-3-(2-(methylthio)-4-((3- oxo-3,4-dihydropyrido[2,3-b]pyrazin-8- yl)oxy)phenyl)urea |
| (bx) | 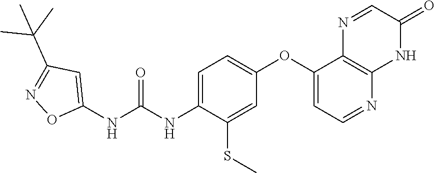 [SMILES: CSc1cc(Oc2ccnc3[nH]c(=O)cnc23)ccc1NC(=O)Nc1cc(C(C)(C)C)no1] | 1-(3-(tert-butyl)isoxazol-5-yl)-3-(2- (methylthio)-4-((3-oxo-3,4- dihydropyrido[2,3-b]pyrazin-8- yl)oxy)phenyl)urea |
| (by) | 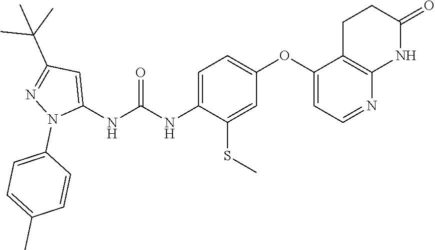 [SMILES: CSc1cc(Oc2ccnc3c2CCC(=O)N3)ccc1NC(=O)Nc1cc(C(C)(C)C)nn1-c1ccc(C)cc1] | 1-(3-(tert-butyl)-1-(p-tolyl)-1H-pyrazol-5- yl)-3-(2-(methylthio)-4-((7-oxo-5,6,7,8- tetrahydro-1,8-naphthyridin-4- yl)oxy)phenyl)urea |
| (bz) | 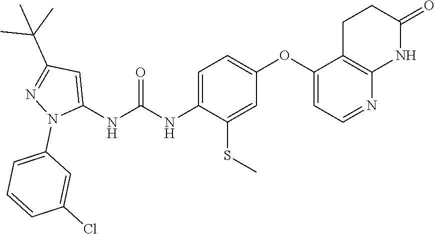 [SMILES: CSc1cc(Oc2ccnc3c2CCC(=O)N3)ccc1NC(=O)Nc1cc(C(C)(C)C)nn1-c1cccc(Cl)c1] | 1-(3-(tert-butyl)-1-(3-chlorophenyl)-1H- pyrazol-5-yl)-3-(2-(methylthio)-4-((7- oxo-5,6,7,8-tetrahydro-1,8-naphthyridin- 4-yl)oxy)phenyl)urea |
| (ca) | 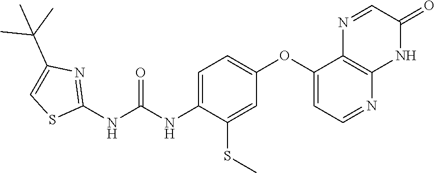 [SMILES: CSc1cc(Oc2ccnc3[nH]c(=O)cnc23)ccc1NC(=O)Nc1nc(C(C)(C)C)cs1] | 1-(4-(tert-butyl)thiazol-2-yl)-3-(2- (methylthio)-4-((3-oxo-3,4- dihydropyrido[2,3-b]pyrazin-8- yl)oxy)phenyl)urea |
| (cb) | 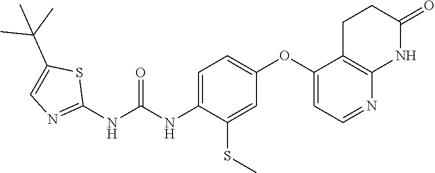 [SMILES: CSc1cc(Oc2ccnc3c2CCC(=O)N3)ccc1NC(=O)Nc1ncc(C(C)(C)C)s1] | 1-(5-(tert-butyl)thiazol-2-yl)-3-(2- (methylthio)-4-((7-oxo-5,6,7,8- tetrahydro-1,8-naphthyridin-4- yl)oxy)phenyl)urea |
| (cc) | 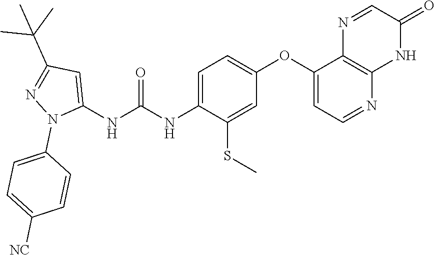 [SMILES: CSc1cc(Oc2ccnc3[nH]c(=O)cnc23)ccc1NC(=O)Nc1cc(C(C)(C)C)nn1-c1ccc(C#N)cc1] | 1-(3-(tert-butyl)-1-(4-cyanophenyl)-1H- pyrazol-5-yl)-3-(2-(methylthio)-4-((3- oxo-3,4-dihydropyrido[2,3-b]pyrazin-8- yl)oxy)phenyl)urea |
| (cd) | 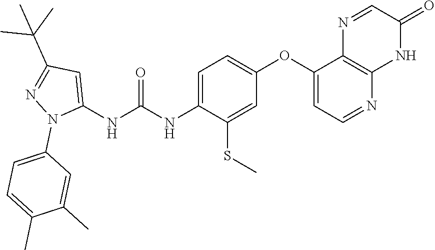 [SMILES: CSc1cc(Oc2ccnc3[nH]c(=O)cnc23)ccc1NC(=O)Nc1cc(C(C)(C)C)nn1-c1ccc(C)c(C)c1] | 1-(3-(tert-butyl)-1-(3,4-dimethylphenyl)- 1H-pyrazol-5-yl)-3-(2-(methylthio)-4-((3- oxo-3,4-dihydropyrido[2,3-b]pyrazin-8- yl)oxy)phenyl)urea |
| (ce) | 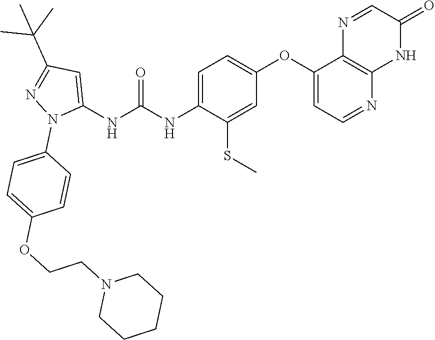 [SMILES: CSc1cc(Oc2ccnc3[nH]c(=O)cnc23)ccc1NC(=O)Nc1cc(C(C)(C)C)nn1-c1ccc(OCCN2CCCCC2)cc1] | 1-(3-(tert-butyl)-1-(4-(2-(piperidin-1- yl)ethoxy)phenyl)-1H-pyrazol-5-yl)-3-(2- (methylthio)-4-((3-oxo-3,4- dihydropyrido[2,3-b]pyrazin-8- yl)oxy)phenyl)urea |
| (cf) | 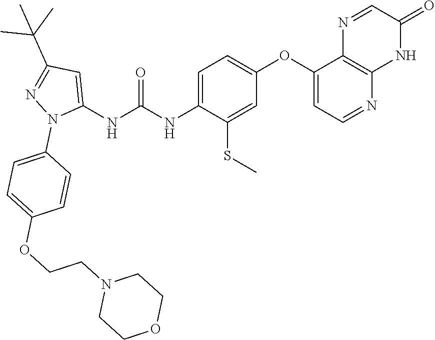 [SMILES: CSc1cc(Oc2ccnc3[nH]c(=O)cnc23)ccc1NC(=O)Nc1cc(C(C)(C)C)nn1-c1ccc(OCCN2CCOCC2)cc1] | 1-(3-(tert-butyl)-1-(4-(2- morpholinoethoxy)phenyl)-1H-pyrazol-5- yl)-3-(2-(methylthio)-4-((3-oxo-3,4- dihydropyrido[2,3-b]pyrazin-8- yl)oxy)phenyl)urea |
| (cg) | 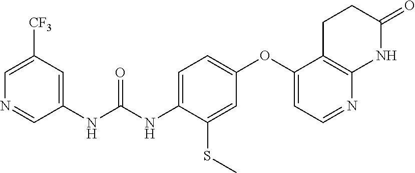 [SMILES: CSc1cc(Oc2ccnc3c2CCC(=O)N3)ccc1NC(=O)Nc1cncc(C(F)(F)F)c1] | 1-(2-(methylthio)-4-((7-oxo-5,6,7,8- tetrahydro-1,8-naphthyridin-4- yl)oxy)phenyl)-3-(5- (trifluoromethyl)pyridin-3-yl)urea |
| (ch) | 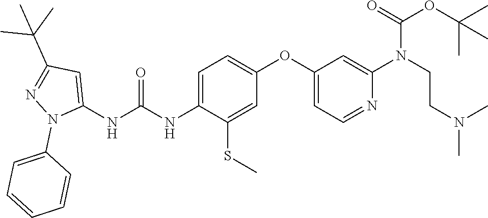 [SMILES: CSc1cc(Oc2ccnc(N(CCN(C)C)C(=O)OC(C)(C)C)c2)ccc1NC(=O)Nc1cc(C(C)(C)C)nn1-c1ccccc1] | tert-butyl (4-(4-(3-(3-(tert-butyl)-1- phenyl-1H-pyrazol-5-yl)ureido)-3- (methylthio)phenoxy)pyridin-2-yl)(2- (dimethylamino)ethyl)carbamate |
| (ci) | 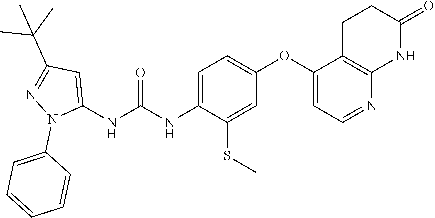 [SMILES: CSc1cc(Oc2ccnc3c2CCC(=O)N3)ccc1NC(=O)Nc1cc(C(C)(C)C)nn1-c1ccccc1] | 1-(3-(tert-butyl)-1-phenyl-1H-pyrazol-5- yl)-3-(2-(methylthio)-4-((2-oxo-1,2,3,4- tetrahydroquinolin-5-yl)oxy)phenyl)urea |
| (cj) | 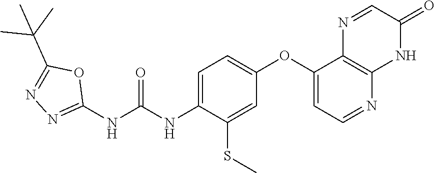 [SMILES: CSc1cc(Oc2ccnc3[nH]c(=O)cnc23)ccc1NC(=O)Nc1nnc(C(C)(C)C)o1] | 1-(5-(tert-butyl)-1,3,4-oxadiazol-2-yl)-3- (2-(methylthio)-4-((3-oxo-3,4- dihydropyrido[2,3-b]pyrazin-8- yl)oxy)phenyl)urea |
| (ck) | 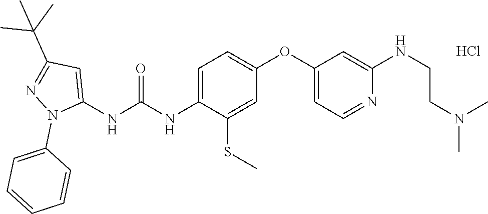 [SMILES: CSc1cc(Oc2ccnc(NCCN(C)C)c2)ccc1NC(=O)Nc1cc(C(C)(C)C)nn1-c1ccccc1 ; Cl] | 1-(3-(tert-butyl)-1-phenyl-1H-pyrazol-5- yl)-3-(4-((2-((2- (dimethylamino)ethyl)amino)pyridin-4- yl)oxy)-2-(methylthio)phenyl)urea hydrochloride |
| (cl) | 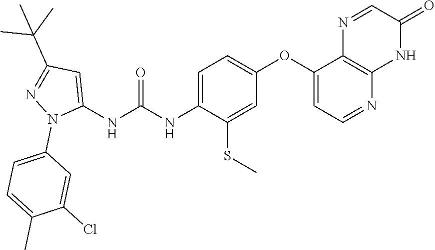 [SMILES: CSc1cc(Oc2ccnc3[nH]c(=O)cnc23)ccc1NC(=O)Nc1cc(C(C)(C)C)nn1-c1ccc(C)c(Cl)c1] | 1-(3-(tert-butyl)-1-(3-chloro-4- methylphenyl)-1H-pyrazol-5-yl)-3-(2- (methylthio)-4-((3-oxo-3,4- dihydropyrido[2,3-b]pyrazin-8- yl)oxy)phenyl)urea |
| (cm) | 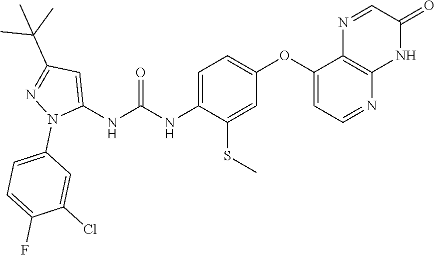 [SMILES: CSc1cc(Oc2ccnc3[nH]c(=O)cnc23)ccc1NC(=O)Nc1cc(C(C)(C)C)nn1-c1ccc(F)c(Cl)c1] | 1-(3-(tert-butyl)-1-(3-chloro-4- fluorophenyl)-1H-pyrazol-5-yl)-3-(2- (methylthio)-4-((3-oxo-3,4- dihydropyrido[2,3-b]pyrazin-8- yl)oxy)phenyl)urea |
| (cn) | 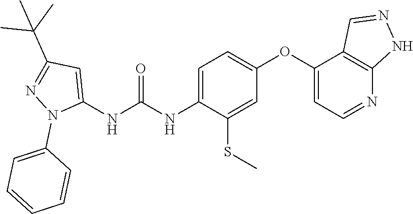 [SMILES: CSc1cc(Oc2ccnc3[nH]ncc23)ccc1NC(=O)Nc1cc(C(C)(C)C)nn1-c1ccccc1] | 1-(4-((1H-indazol-4-yl)oxy)-2- (methylthio)phenyl)-3-(3-(tert-butyl)-1- phenyl-1H-pyrazol-5-yl)urea |
| (co) | 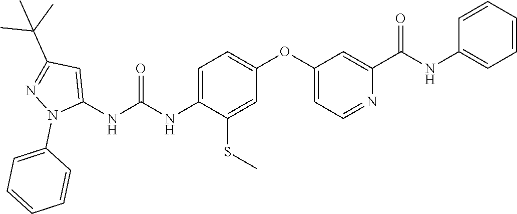 [SMILES: CSc1cc(Oc2ccnc(C(=O)Nc3ccccc3)c2)ccc1NC(=O)Nc1cc(C(C)(C)C)nn1-c1ccccc1] | 4-(4-(3-(3-(tert-butyl)-1-phenyl-1H- pyrazol-5-yl)ureido)-3- (methylthio)phenoxy)-N- phenylpicolinamide |
| (cp) | 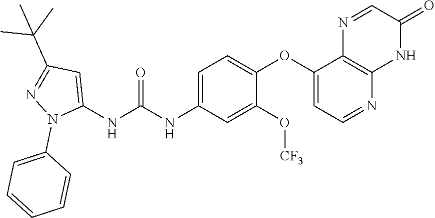 [SMILES: CC(C)(C)c1cc(NC(=O)Nc2ccc(Oc3ccnc4[nH]c(=O)cnc34)c(OC(F)(F)F)c2)n(-c2ccccc2)n1] | 1-(3-(tert-butyl)-1-phenyl-1H-pyrazol-5- yl)-3-(4-((3-oxo-3,4-dihydropyrido[2,3- b]pyrazin-8-yl)oxy)-3- (trifluoromethoxy)phenyl)urea |
| (cq) | 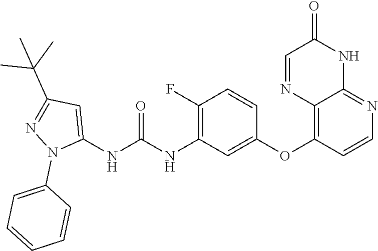 [SMILES: CC(C)(C)c1cc(NC(=O)Nc2cc(Oc3ccnc4[nH]c(=O)cnc34)ccc2F)n(-c2ccccc2)n1] | 1-(3-(tert-butyl)-1-phenyl-1H-pyrazol-5- yl)-3-(2-fluoro-5-((3-oxo- 3,4-dihydropyrido[2,3-b]pyrazin-8- yl)oxy)phenyl)urea |
| (cr) | 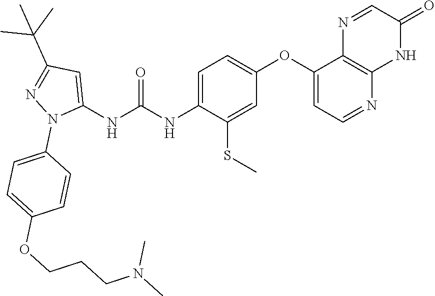 [SMILES: CSc1cc(Oc2ccnc3[nH]c(=O)cnc23)ccc1NC(=O)Nc1cc(C(C)(C)C)nn1-c1ccc(OCCCN(C)C)cc1] | 1-(3-(tert-butyl)-1-(4-(3- (dimethylamino)propoxy)phenyl)-1H- pyrazol-5-yl)-3-(2-(methylthio)-4-((3- oxo-3,4-dihydropyrido[2,3-b]pyrazin-8- yl)oxy)phenyl)urea |
| (cs) | 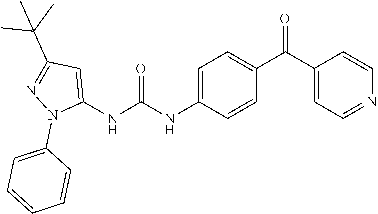 [SMILES: CC(C)(C)c1cc(NC(=O)Nc2ccc(C(=O)c3ccncc3)cc2)n(-c2ccccc2)n1] | 1-(3-(tert-butyl)-1-phenyl-1H-pyrazol-5- yl)-3-(4-isonicotinoylphenyl)urea |
| (ct) | 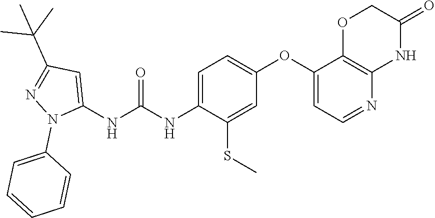 [SMILES: CSc1cc(Oc2ccnc3c2OCC(=O)N3)ccc1NC(=O)Nc1cc(C(C)(C)C)nn1-c1ccccc1] | 1-(3-(tert-butyl)-1-phenyl-1H-pyrazol-5- yl)-3-(2-(methylthio)-4-((3-oxo-3,4- dihydro-2H-pyrido[3,2-b][1,4]oxazin-8- yl)oxy)phenyl)urea |
| (cu) | 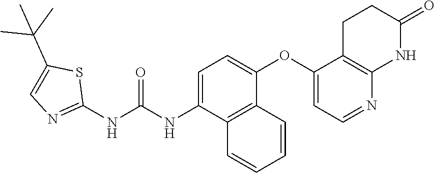 [SMILES: CC(C)(C)c1cnc(NC(=O)Nc2ccc(Oc3ccnc4c3CCC(=O)N4)c3ccccc23)s1] | 1-(5-(tert-butyl)thiazol-2-yl)-3-(4-((7-oxo- 5,6,7,8-tetrahydro-1,8-naphthyridin-4- yl)oxy)naphthalen-1-yl)urea |
| (cv) | 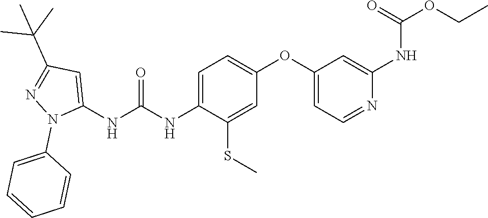 [SMILES: CCOC(=O)Nc1cc(Oc2ccc(NC(=O)Nc3cc(C(C)(C)C)nn3-c3ccccc3)c(SC)c2)ccn1] | ethyl (4-(4-(3-(3-(tert-butyl)-phenyl- 1H-pyrazol-5-yl)ureido)-3- (methylthio)phenoxy)pyridin-2- yl)carbamate |
| (cw) | 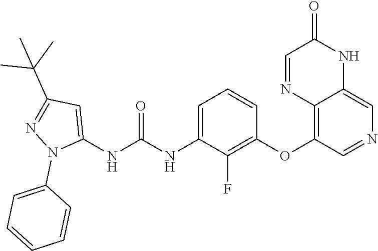 [SMILES: CC(C)(C)c1cc(NC(=O)Nc2cccc(Oc3cncc4[nH]c(=O)cnc34)c2F)n(-c2ccccc2)n1] | 1-(3-(tert-butyl)-1-phenyl-1H-pyrazol-5- yl)-3-(2-fluoro-3-((3-oxo-3,4- dihydropyrido[2,3-b]pyrazin-8- yl)oxy)phenyl)urea |
| (cx) | 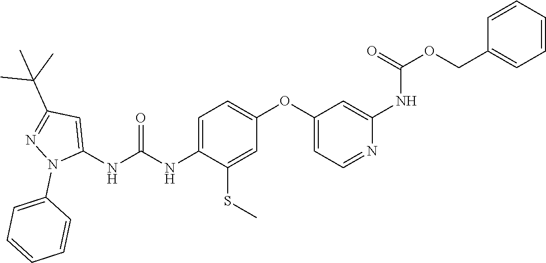 [SMILES: CSc1cc(Oc2ccnc(NC(=O)OCc3ccccc3)c2)ccc1NC(=O)Nc1cc(C(C)(C)C)nn1-c1ccccc1] | benzyl (4-(4-(3-(3-(tert-butyl)-phenyl- 1H-pyrazol-5-yl)ureido)-3- (methylthio)phenoxy)pyridin-2- yl)carbamate |
| (cy) | 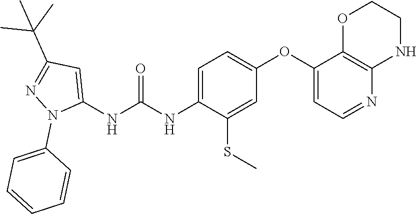 [SMILES: CSc1cc(Oc2ccnc3c2OCCN3)ccc1NC(=O)Nc1cc(C(C)(C)C)nn1-c1ccccc1] | 1-(3-(tert-butyl)-1-phenyl-1H-pyrazol-5- yl)-3-(4-((3,4-dihydro-2H-pyrido[3,2- b][1,4]oxazin-8-yl)oxy)-2- (methylthio)phenyl)urea |
| (cz) | 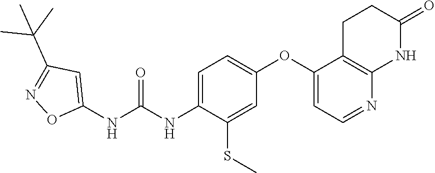 [SMILES: CSc1cc(Oc2ccnc3c2CCC(=O)N3)ccc1NC(=O)Nc1cc(C(C)(C)C)no1] | 1-(3-(tert-butyl)isoxazol-5-yl)-3-(2- (methylthio)-4-((7-oxo-5,6,7,8- tetrahydro-1,8-naphthyridin-4- yl)oxy)phenyl)urea |
| (da) | 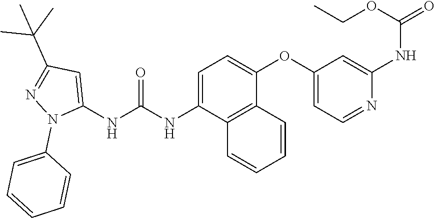 [SMILES: CCOC(=O)Nc1cc(Oc2ccc(NC(=O)Nc3cc(C(C)(C)C)nn3-c3ccccc3)c3ccccc23)ccn1] | ethyl (4-((4-(3-(3-(tert-butyl)-phenyl- 1H-pyrazol-5-yl)ureido)naphthalen-1- yl)oxy)pyridin-2-yl)carbamate |
| (db) | 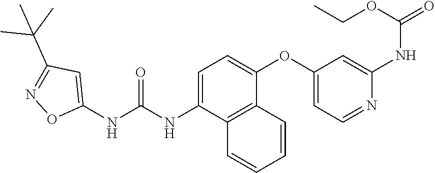 [SMILES: CCOC(=O)Nc1cc(Oc2ccc(NC(=O)Nc3cc(C(C)(C)C)no3)c3ccccc23)ccn1] | ethyl (4-((4-(3-(3-(tert-butyl)isoxazol-5- yl)ureido)naphthalen-1-yl)oxy)pyridin-2- yl)carbamate |
| (dc) | 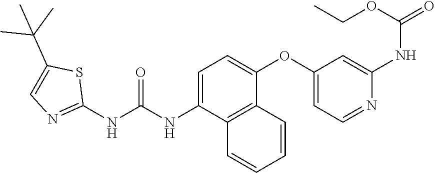 [SMILES: CCOC(=O)Nc1cc(Oc2ccc(NC(=O)Nc3ncc(C(C)(C)C)s3)c3ccccc23)ccn1] | ethyl (4-((4-(3-(5-(tert-butyl)thiazol-2- yl)ureido)naphthalen-1-yl)oxy)pyridin-2- yl)carbamate |
| (dd) | 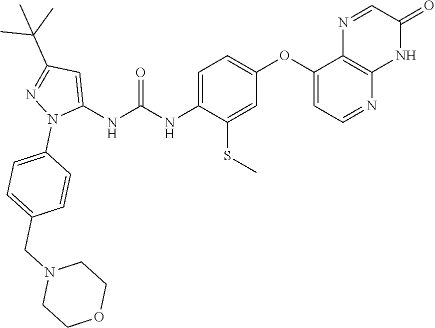 [SMILES: CSc1cc(Oc2ccnc3[nH]c(=O)cnc23)ccc1NC(=O)Nc1cc(C(C)(C)C)nn1-c1ccc(CN2CCOCC2)cc1] | 1-(3-(tert-butyl)-1-(4- (morpholinomethyl)phenyl)-1H-pyrazol- 5-yl)-3-(2-(methylthio)-4-((3-oxo-3,4- dihydropyrido[2,3-b]pyrazin-8- yl)oxy)phenyl)urea |
| (de) | 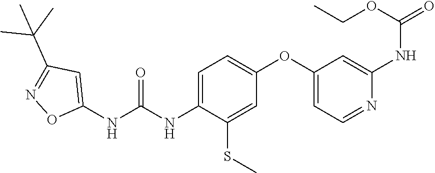 [SMILES: CCOC(=O)Nc1cc(Oc2ccc(NC(=O)Nc3cc(C(C)(C)C)no3)c(SC)c2)ccn1] | ethyl (4-(4-(3-(3-(tert-butyl)isoxazol-5- yl)ureido)-3- (methylthio)phenoxy)pyridin-2- yl)carbamate |
| (df) | 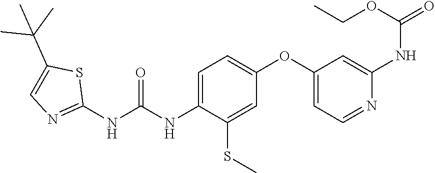 [SMILES: CCOC(=O)Nc1cc(Oc2ccc(NC(=O)Nc3ncc(C(C)(C)C)s3)c(SC)c2)ccn1] | ethyl (4-(4-(3-(5-(tert-butyl)thiazol-2- yl)ureido)-3- (methylthio)phenoxy)pyridin-2- yl)carbamate |
| (dg) | 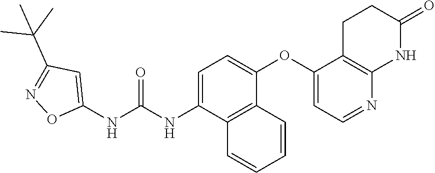 [SMILES: CC(C)(C)c1cc(NC(=O)Nc2ccc(Oc3ccnc4c3CCC(=O)N4)c3ccccc23)on1] | 1-(3-(tert-butyl)isoxazol-5-yl)-3-(4-((7- oxo-5,6,7,8-tetrahydro-1,8-naphthyridin- 4-yl)oxy)naphthalen-1-yl)urea |
| (dh) | 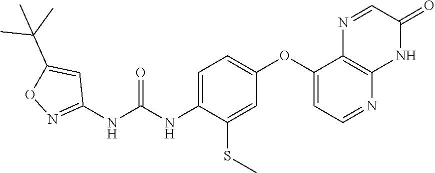 [SMILES: CSc1cc(Oc2ccnc3[nH]c(=O)cnc23)ccc1NC(=O)Nc1cc(C(C)(C)C)on1] | 1-(5-(tert-butyl)isoxazol-3-yl)-3-(2- (methylthio)-4-((3-oxo-3,4- dihydropyrido[2,3-b]pyrazin-8- yl)oxy)phenyl)urea |
| (di) | 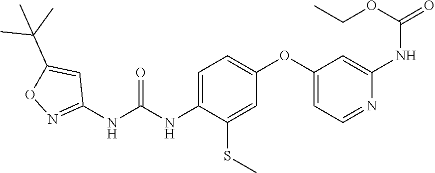 [SMILES: CCOC(=O)Nc1cc(Oc2ccc(NC(=O)Nc3cc(C(C)(C)C)on3)c(SC)c2)ccn1] | ethyl (4-(4-(3-(5-(tert-butyl)isoxazol-3- yl)ureido)-3- (methylthio)phenoxy)pyridin-2- yl)carbamate |
| (dj) | 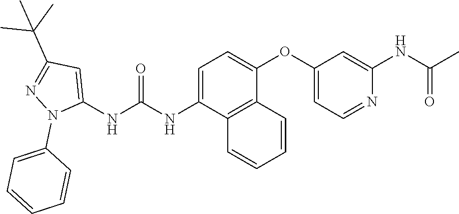 [SMILES: CC(=O)Nc1cc(Oc2ccc(NC(=O)Nc3cc(C(C)(C)C)nn3-c3ccccc3)c3ccccc23)ccn1] | N-(4-((4-(3-(3-(tert-butyl)-1-phenyl-1H- pyrazol-5-yl)ureido)naphthalen-1- yl)oxy)pyridin-2-yl)acetamide |
| (dk) | 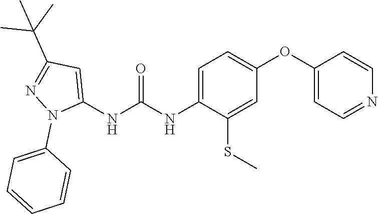 [SMILES: CSc1cc(Oc2ccncc2)ccc1NC(=O)Nc1cc(C(C)(C)C)nn1-c1ccccc1] | 1-(3-(tert-butyl)-1-phenyl-1H-pyrazol-5- yl)-3-(2-(methylthio)-4-(pyridin-4- yloxy)phenyl)urea |
| (dl) | 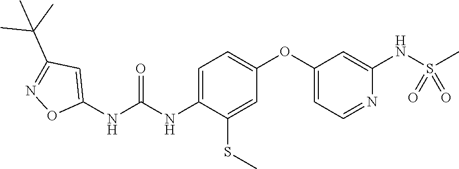 [SMILES: CSc1cc(Oc2ccnc(NS(C)(=O)=O)c2)ccc1NC(=O)Nc1cc(C(C)(C)C)no1] | N-(4-(4-(3-(3-(tert-butyl)isoxazol-5- yl)ureido)-3- (methylthio)phenoxy)pyridin-2- yl)methanesulfonamide |
| (dm) | 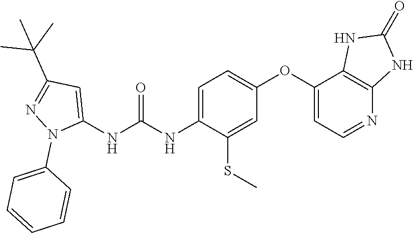 [SMILES: CSc1cc(Oc2ccnc3[nH]c(=O)[nH]c23)ccc1NC(=O)Nc1cc(C(C)(C)C)nn1-c1ccccc1] | 1-(3-(tert-butyl)-1-phenyl-1H-pyrazol-5- yl)-3-(2-(methylthio)-4-((2-oxo-2,3- dihydro-1H-imidazo[4,5-b]pyridin-7- yl)oxy)phenyl)urea |
| (dn) | 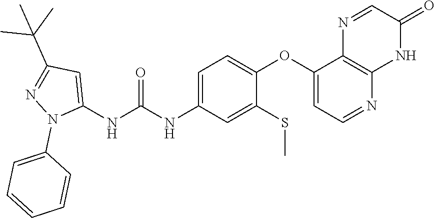 [SMILES: CSc1cc(NC(=O)Nc2cc(C(C)(C)C)nn2-c2ccccc2)ccc1Oc1ccnc2[nH]c(=O)cnc12] | 1-(3-(tert-butyl)-1-phenyl-1H-pyrazol-5- yl)-3-(3-(methylthio)-4-((3-oxo-3,4- dihydropyrido[2,3-b]pyrazin-8- yl)oxy)phenyl)urea |
| (do) | 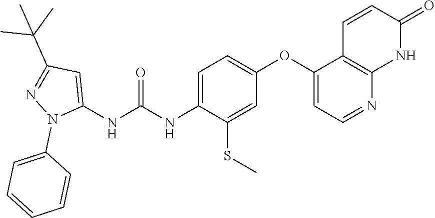 [SMILES: CSc1cc(Oc2ccnc3[nH]c(=O)ccc23)ccc1NC(=O)Nc1cc(C(C)(C)C)nn1-c1ccccc1] | 1-(3-(tert-butyl)-1-phenyl-1H-pyrazol-5- yl)-3-(2-(methylthio)-4-((7-oxo-7,8- dihydro-1,8-naphthyridin-4- yl)oxy)phenyl)urea |
| (dp) | 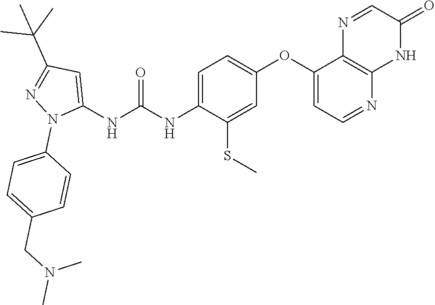 [SMILES: CSc1cc(Oc2ccnc3[nH]c(=O)cnc23)ccc1NC(=O)Nc1cc(C(C)(C)C)nn1-c1ccc(CN(C)C)cc1] | 1-(3-(tert-butyl)-1-(4- ((dimethylamino)methyl)phenyl)-1H- pyrazol-5-yl)-3-(2-(methylthio)-4-((3- oxo-3,4-dihydropyrido[2,3-b]pyrazin-8- yl)oxy)phenyl)urea |
| (dq) | 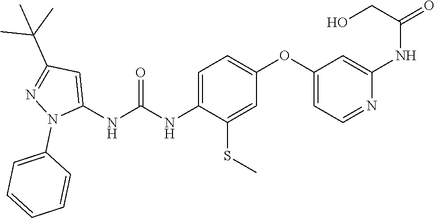 [SMILES: CSc1cc(Oc2ccnc(NC(=O)CO)c2)ccc1NC(=O)Nc1cc(C(C)(C)C)nn1-c1ccccc1] | N-(4-(4-(3-(3-(tert-butyl)-1-phenyl-1H- pyrazol-5-yl)ureido)-3- (methylthio)phenoxy)pyridin-2-yl)-2- hydroxyacetamide. |

In a particular aspect, the invention also relates to a compound represented by a structure selected in the group consisting of the following list or a pharmaceutically acceptable salt thereof. The invention also relates to a compound represented by a structure a structure selected in the group consisting of the following list or a pharmaceutically acceptable salt thereof or a pharmaceutical or veterinary composition comprising such a compound for use as a medicament, preferably for use in treating, ameliorating, delaying, curing and/or preventing a disease or condition associated with muscle cells and/or satellite cells:

(g), (l), (n), (s), (v), (ab), (ai), (aj), (am), (an), (aq), (as), (aw), (az), (ba), (bb), (bd), (be), (bi), (bl), (bm), (bo), (bp), (bq), (bt), (bw), (bx), (by), (bz), (cb), (cc), (cd), (ce), (cf), (ck), (cl), (cm), (cp), (cq), (cr), (ct), (cu), (cv), (cx), (cy), (cz), (da), (db), (dc), (dd), (de), (df), (dg), (dh), (di), (dj), (dn) and (do)

preferably

(n), (v), (am), (an), (as), (az), (ba), (bb), (bd), (be), (bi), (bm), (bo), (bp), (bq), (bw), (by), (bz), (cb), (cc), (cd), (ce), (cl), (cr), (ct), (cu), (cv), (da), (db), (dc), (dd), (de), (df), (dg), (dh), (di), (dj), and (do).

The present invention further relates to the use of a compound represented by a structure selected in the group consisting of the following list or a pharmaceutically acceptable salt thereof for the manufacture of a medicament, in particular for treating, ameliorating, delaying, curing and/or preventing a disease or condition associated with muscle cells and/or satellite cells:

(g), (l), (n), (s), (v), (ab), (ai), (aj), (am), (an), (aq), (as), (aw), (az), (ba), (bb), (bd), (be), (bi), (bl), (bm), (bo), (bp), (bq), (bt), (bw), (bx), (by), (bz), (cb), (cc), (cd), (ce), (cf), (ck), (cl), (cm), (cp), (cq), (cr), (ct), (cu), (cv), (cx), (cy), (cz), (da), (db), (dc), (dd), (de), (df), (dg), (dh), (di), (dj), (dn) and (do)

preferably

(n), (v), (am), (an), (as), (az), (ba), (bb), (bd), (be), (bi), (bm), (bo), (bp), (bq), (bw), (by), (bz), (cb), (cc), (cd), (ce), (cl), (cr), (ct), (cu), (cv), (da), (db), (dc), (dd), (de), (df), (dg), (dh), (di), (dj), and (do).

The present invention also relates to a method for treating, ameliorating, delaying, curing and/or preventing a disease or condition associated with muscle cells and/or satellite cells in a subject in need thereof comprising administering a therapeutically effective amount of a compound represented by a structure selected in the group consisting of the following list or a pharmaceutically acceptable salt thereof, thereby treating, ameliorating, delaying, curing and/or preventing a disease or condition associated with muscle cells and/or satellite cells;

(g), (l), (n), (s), (v), (ab), (ai), (aj), (am), (an), (aq), (as), (aw), (az), (ba), (bb), (bd), (be), (bi), (bl), (bm), (bo), (bp), (bq), (bt), (bw), (bx), (by), (bz), (cb), (cc), (cd), (ce), (cf), (ck), (cl), (cm), (cp), (cq), (cr), (ct), (cu), (cv), (cx), (cy), (cz), (da), (db), (dc), (dd), (de), (df), (dg), (dh), (di), (dj), (dn) and (do)

preferably

(n), (v), (am), (an), (as), (az), (ba), (bb), (bd), (be), (bi), (bm), (bo), (bp), (bq), (bw), (by), (bz), (cb), (cc), (cd), (ce), (cl), (cr), (ct), (cu), (cv), (da), (db), (dc), (dd), (de), (df), (dg), (dh), (di), (dj), and (do).

In a particular aspect, the invention also relates to
a compound or a pharmaceutically acceptable salt thereof or a pharmaceutical or veterinary composition comprising such a compound and their use as a medicament, preferably for use in treating, ameliorating, delaying, curing and/or preventing a disease or condition associated with muscle cells and/or satellite cell,
the use of a compound represented by a structure selected in the group consisting of one of the following lists or a pharmaceutically acceptable salt thereof for the manufacture of a medicament, in particular for treating, ameliorating, delaying, curing and/or preventing a disease or condition associated with muscle cells and/or satellite cells; or
a method for treating, ameliorating, delaying, curing and/or preventing a disease or condition associated with muscle cells and/or satellite cells in a subject in need thereof comprising administering a effective active amount of a compound represented by a structure selected in the group consisting of one of the following lists or a pharmaceutically acceptable salt thereof, thereby treating, ameliorating, delaying, curing and/or preventing a disease or condition associated with muscle cells and/or satellite cells

wherein the compound is selected in one of the following lists:

1)
1-(3-(tert-butyl)-1-phenyl-1H-pyrazol-5-yl)-3-(2-(methylthio)-4-((7-oxo-5,6,7,8-tetrahydro-1,8-naphthyridin-4-yl)oxy)phenyl)urea;
1-(3-(tert-butyl)-1-phenyl-1H-pyrazol-5-yl)-3-(2-(methylthio)-4-((5,6,7,8-tetrahydro-1,8-naphthyridin-4-yl)oxy)phenyl)urea;
1-(3-(tert-butyl)-1-(p-tolyl)-1H-pyrazol-5-yl)-3-(2-(methylthio)-4-((7-oxo-5,6,7,8-tetrahydro-1,8-naphthyridin-4-yl)oxy)phenyl)urea;
1-(5-(tert-butyl)thiazol-2-yl)-3-(2-(methylthio)-4-((7-oxo-5,6,7,8-tetrahydro-1,8-naphthyridin-4-yl)oxy)phenyl)urea;
1-(3-(tert-butyl)-1-phenyl-1H-pyrazol-5-yl)-3-(2-(methylthio)-4-((3-oxo-3,4-dihydro-2H-pyrido[3,2-b][1,4]oxazin-8-yl)oxy)phenyl)urea;
1-(5-(tert-butyl)thiazol-2-yl)-3-(4-((7-oxo-5,6,7,8-tetrahydro-1,8-naphthyridin-4-yl)oxy)naphthalen-1-yl)urea;
1-(3-(tert-butyl)-1-phenyl-1H-pyrazol-5-yl)-3-(4-((3,4-dihydro-2H-pyrido[3,2-b][1,4]oxazin-8-yl)oxy)-2-(methylthio)phenyl)urea;
1-(3-(tert-butyl)isoxazol-5-yl)-3-(2-(methylthio)-4-((7-oxo-5,6,7,8-tetrahydro-1,8-naphthyridin-4-yl)oxy)phenyl)urea;
1-(3-(tert-butyl)isoxazol-5-yl)-3-(4-((7-oxo-5,6,7,8-tetrahydro-1,8-naphthyridin-4-yl)oxy)naphthalen-1-yl)urea;
1-(3-(tert-butyl)-1-phenyl-1H-pyrazol-5-yl)-3-(2-(methylthio)-4-((7-oxo-7,8-dihydro-1,8-naphthyridin-4-yl)oxy)phenyl)urea;
1-(5-(tert-butyl)thiazol-2-yl)-3-(4-((3-oxo-3,4-dihydropyrido[2,3-b]pyrazin-8-yl)oxy)naphthalen-1-yl)urea;
1-(3-(tert-butyl)isoxazol-5-yl)-3-(2-(methylthio)-4-((3-oxo-3,4-dihydropyrido[2,3-b]pyrazin-8-yl)oxy)phenyl)urea;
1-(5-(tert-butyl)thiazol-2-yl)-3-(2-(methylthio)-4-((7-oxo-5,6,7,8-tetrahydro-1,8-naphthyridin-4-yl)oxy)phenyl)urea;
1-(5-(tert-butyl)thiazol-2-yl)-3-(4-((7-oxo-5,6,7,8-tetrahydro-1,8-naphthyridin-4-yl)oxy)naphthalen-1-yl)urea;
1-(3-(tert-butyl)isoxazol-5-yl)-3-(2-(methylthio)-4-((7-oxo-5,6,7,8-tetrahydro-1,8-naphthyridin-4-yl)oxy)phenyl)urea;
ethyl (4-(4-(3-(3-(tert-butyl)isoxazol-5-yl)ureido)-3-(methylthio)phenoxy)pyridin-2-yl)carbamate;
ethyl (4-(4-(3-(5-(tert-butyl)thiazol-2-yl)ureido)-3-(methylthio)phenoxy)pyridin-2-yl)carbamate;
1-(3-(tert-butyl)isoxazol-5-yl)-3-(4-((7-oxo-5,6,7,8-tetrahydro-1,8-naphthyridin-4-yl)oxy)naphthalen-1-yl)urea;
ethyl (4-(4-(3-(5-(tert-butyl)isoxazol-3-yl)ureido)-3-(methylthio)phenoxy)pyridin-2-yl)carbamate;
1-(3-(tert-butyl)-1-phenyl-1H-pyrazol-5-yl)-3-(4-((3-oxo-3,4-dihydropyrido[2,3-b]pyrazin-8-yl)oxy)-2-(trifluoromethyl)phenyl)urea;
1-(3-(tert-butyl)-1-(p-tolyl)-1H-pyrazol-5-yl)-3-(4-((3-oxo-3,4-dihydropyrido[2,3-b]pyrazin-8-yl)oxy)-2-(trifluoromethyl)phenyl)urea;
1-(3-(tert-butyl)-1-phenyl-1H-pyrazol-5-yl)-3-(2-(ethylthio)-4-((3-oxo-3,4-dihydropyrido[2,3-b]pyrazin-8-yl)oxy)phenyl)urea;
1-(3-(tert-butyl)-1-phenyl-1H-pyrazol-5-yl)-3-(2,3-difluoro-4-((3-oxo-3,4-dihydropyrido[2,3-b]pyrazin-8-yl)oxy)phenyl)urea;
1-(3-(tert-butyl)-1-phenyl-1H-pyrazol-5-yl)-3-(2,5-difluoro-4-((3-oxo-3,4-dihydropyrido[2,3-b]pyrazin-8-yl)oxy)phenyl)urea;
1-(3-(tert-butyl)-1-(4-methoxyphenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(3-chlorophenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(3-methoxyphenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(3-fluorophenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(quinolin-6-yl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(4-cyanophenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(3,4-dimethylphenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(4-(2-(piperidin-1-yl)ethoxy)phenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(4-(2-morpholinoethoxy)phenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(3-chloro-4-methylphenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(3-chloro-4-fluorophenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(4-(3-(dimethylamino)propoxy)phenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(4-(morpholinomethyl)phenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(quinolin-6-yl)-1H-pyrazol-5-yl)-3-(2-fluoro-4-((3-oxo-3,4-dihydropyrido[2,3-b]pyrazin-8-yl)oxy)phenyl)urea;
1-(3-(tert-butyl)-1-(quinolin-6-yl)-1H-pyrazol-5-yl)-3-(4-((3-oxo-3,4-dihydropyrido[2,3-b]pyrazin-8-yl)oxy)naphthalen-1-yl)urea;
1-(3-(tert-butyl)-1-(quinolin-6-yl)-1H-pyrazol-5-yl)-3-(2-(methylthio)-4-((3-oxo-3,4-dihydropyrido[2,3-b]pyrazin-8-yl)oxy)phenyl)urea;
5-(4-(3-(3-(tert-butyl)-1-phenyl-1H-pyrazol-5-yl)ureido)-3-(methylthio)phenoxy)-N-methylnicotinamide;
N-(4-(4-(3-(3-(tert-butyl)-1-phenyl-1H-pyrazol-5-yl)ureido)-3-(methylthio)phenoxy)pyridin-2-yl)acetamide;
tert-butyl (4-(4-(3-(3-(tert-butyl)-1-phenyl-1H-pyrazol-5-yl)ureido)-3-(methylthio)phenoxy)pyridin-2-yl)carbamate;
tert-butyl (2-((4-(4-(3-(3-(tert-butyl)-1-phenyl-1H-pyrazol-5-yl)ureido)-3-(methylthio)phenoxy)pyridin-2-yl)amino)-2-oxoethyl)(methyl)carbamate;
N-(4-(4-(3-(3-(tert-butyl)-1-phenyl-1H-pyrazol-5-yl)ureido)-3-(methylthio)phenoxy)pyridin-2-yl)-2-(methylamino)acetamide;
1-(3-(tert-butyl)-1-phenyl-1H-pyrazol-5-yl)-3-(4-((2-((2-(dimethylamino)ethyl)amino)pyridin-4-yl)oxy)-2-(methylthio)phenyl)urea hydrochloride;
ethyl (4-(4-(3-(3-(tert-butyl)-1-phenyl-1H-pyrazol-5-yl)ureido)-3-(methylthio)phenoxy)pyridin-2-yl)carbamate;
benzyl (4-(4-(3-(3-(tert-butyl)-1-phenyl-1H-pyrazol-5-yl)ureido)-3-(methylthio)phenoxy)pyridin-2-yl)carbamate;
ethyl (4-(4-(3-(3-(tert-butyl)isoxazol-5-yl)ureido)-3-(methylthio)phenoxy)pyridin-2-yl)carbamate;
ethyl (4-(4-(3-(5-(tert-butyl)thiazol-2-yl)ureido)-3-(methylthio)phenoxy)pyridin-2-yl)carbamate;
ethyl (4-(4-(3-(5-(tert-butyl)isoxazol-3-yl)ureido)-3-(methylthio)phenoxy)pyridin-2-yl)carbamate;
N-(4-(4-(3-(3-(tert-butyl)-1-phenyl-1H-pyrazol-5-yl)ureido)-3-fluorophenoxy)pyridin-2-yl)acetamide;
N-(4-(4-(3-(3-(tert-butyl)-1-phenyl-1H-pyrazol-5-yl)ureido)-3-(methylthio)phenoxy)pyridin-2-yl)acetamide;
tert-butyl (4-(4-(3-(3-(tert-butyl)-1-phenyl-1H-pyrazol-5-yl)ureido)-3-(methylthio)phenoxy)pyridin-2-yl)carbamate;
tert-butyl (2-((4-(4-(3-(3-(tert-butyl)-1-phenyl-1H-pyrazol-5-yl)ureido)-3-(methylthio)phenoxy)pyridin-2-yl)amino)-2-oxoethyl)(methyl)carbamate;
ethyl (4-(4-(3-(3-(tert-butyl)-1-phenyl-1H-pyrazol-5-yl)ureido)-3-(methylthio)phenoxy)pyridin-2-yl)carbamate
ethyl (4-((4-(3-(3-(tert-butyl)-1-phenyl-1H-pyrazol-5-yl)ureido)naphthalen-1-yl)oxy)pyridin-2-yl)carbamate;
ethyl (4-((4-(3-(3-(tert-butyl)isoxazol-5-yl)ureido)naphthalen-1-yl)oxy)pyridin-2-yl)carbamate;
ethyl (4-((4-(3-(5-(tert-butyl)thiazol-2-yl)ureido)naphthalen-1-yl)oxy)pyridin-2-yl)carbamate;
ethyl (4-(4-(3-(3-(tert-butyl)isoxazol-5-yl)ureido)-3-(methylthio)phenoxy)pyridin-2-yl)carbamate;
ethyl (4-(4-(3-(5-(tert-butyl)thiazol-2-yl)ureido)-3-(methylthio)phenoxy)pyridin-2-yl)carbamate;
benzyl (4-(4-(3-(3-(tert-butyl)-1-phenyl-1H-pyrazol-5-yl)ureido)-3-(methylthio)phenoxy)pyridin-2-yl)carbamate;
N-(4-(4-(3-(3-(tert-butyl)-1-phenyl-1H-pyrazol-5-yl)ureido)-3-(methylthio)phenoxy)pyridin-2-yl)-2-hydroxyacetamide; and
tert-butyl (4-(4-(3-(3-(tert-butyl)-1-phenyl-1H-pyrazol-5-yl)ureido)-3-(methylthio)phenoxy)pyridin-2-yl)(2-(dimethylamino)ethyl)carbamate;

2)
1-(3-(tert-butyl)-1-phenyl-1H-pyrazol-5-yl)-3-(2-(methylthio)-4-((7-oxo-5,6,7,8-tetrahydro-1,8-naphthyridin-4-yl)oxy)phenyl)urea;
1-(3-(tert-butyl)-1-phenyl-1H-pyrazol-5-yl)-3-(2-(methylthio)-4-((5,6,7,8-tetrahydro-1,8-naphthyridin-4-yl)oxy)phenyl)urea;
1-(3-(tert-butyl)-1-(p-tolyl)-1H-pyrazol-5-yl)-3-(2-(methylthio)-4-((7-oxo-5,6,7,8-tetrahydro-1,8-naphthyridin-4-yl)oxy)phenyl)urea;
1-(5-(tert-butyl)thiazol-2-yl)-3-(2-(methylthio)-4-((7-oxo-5,6,7,8-tetrahydro-1,8-naphthyridin-4-yl)oxy)phenyl)urea;
1-(3-(tert-butyl)-1-phenyl-1H-pyrazol-5-yl)-3-(2-(methylthio)-4-((3-oxo-3,4-dihydro-2H-pyrido[3,2-b][1,4]oxazin-8-yl)oxy)phenyl)urea;
1-(5-(tert-butyl)thiazol-2-yl)-3-(4-((7-oxo-5,6,7,8-tetrahydro-1,8-naphthyridin-4-yl)oxy)naphthalen-1-yl)urea;
1-(3-(tert-butyl)-1-phenyl-1H-pyrazol-5-yl)-3-(4-((3,4-dihydro-2H-pyrido[3,2-b][1,4]oxazin-8-yl)oxy)-2-(methylthio)phenyl)urea;
1-(3-(tert-butyl)isoxazol-5-yl)-3-(2-(methylthio)-4-((7-oxo-5,6,7,8-tetrahydro-1,8-naphthyridin-4-yl)oxy)phenyl)urea;
1-(3-(tert-butyl)isoxazol-5-yl)-3-(4-((7-oxo-5,6,7,8-tetrahydro-1,8-naphthyridin-4-yl)oxy)naphthalen-1-yl)urea; and
1-(3-(tert-butyl)-1-phenyl-1H-pyrazol-5-yl)-3-(2-(methylthio)-4-((7-oxo-7,8-dihydro-1,8-naphthyridin-4-yl)oxy)phenyl)urea

3)
1-(5-(tert-butyl)thiazol-2-yl)-3-(4-((3-oxo-3,4-dihydropyrido[2,3-b]pyrazin-8-yl)oxy)naphthalen-1-yl)urea;
1-(3-(tert-butyl)isoxazol-5-yl)-3-(2-(methylthio)-4-((3-oxo-3,4-dihydropyrido[2,3-b]pyrazin-8-yl)oxy)phenyl)urea;
1-(5-(tert-butyl)thiazol-2-yl)-3-(2-(methylthio)-4-((7-oxo-5,6,7,8-tetrahydro-1,8-naphthyridin-4-yl)oxy)phenyl)urea;
1-(5-(tert-butyl)thiazol-2-yl)-3-(4-((7-oxo-5,6,7,8-tetrahydro-1,8-naphthyridin-4-yl)oxy)naphthalen-1-yl)urea;
1-(3-(tert-butyl)isoxazol-5-yl)-3-(2-(methylthio)-4-((7-oxo-5,6,7,8-tetrahydro-1,8-naphthyridin-4-yl)oxy)phenyl)urea;
ethyl (4-(4-(3-(3-(tert-butyl)isoxazol-5-yl)ureido)-3-(methylthio)phenoxy)pyridin-2-yl)carbamate;
ethyl (4-(4-(3-(5-(tert-butyl)thiazol-2-yl)ureido)-3-(methylthio)phenoxy)pyridin-2-yl)carbamate;
1-(3-(tert-butyl)isoxazol-5-yl)-3-(4-((7-oxo-5,6,7,8-tetrahydro-1,8-naphthyridin-4-yl)oxy)naphthalen-1-yl)urea; and
ethyl (4-(4-(3-(5-(tert-butyl)isoxazol-3-yl)ureido)-3-(methylthio)phenoxy)pyridin-2-yl)carbamate;

4)
1-(3-(tert-butyl)-1-phenyl-1H-pyrazol-5-yl)-3-(4-((3-oxo-3,4-dihydropyrido[2,3-b]pyrazin-8-yl)oxy)-2-(trifluoromethyl)phenyl)urea;
1-(3-(tert-butyl)-1-(p-tolyl)-1H-pyrazol-5-yl)-3-(4-((3-oxo-3,4-dihydropyrido[2,3-b]pyrazin-8-yl)oxy)-2-(trifluoromethyl)phenyl)urea;
1-(3-(tert-butyl)-1-phenyl-1H-pyrazol-5-yl)-3-(2-(ethylthio)-4-((3-oxo-3,4-dihydropyrido[2,3-b]pyrazin-8-yl)oxy)phenyl)urea;
1-(3-(tert-butyl)-1-phenyl-1H-pyrazol-5-yl)-3-(2,3-difluoro-4-((3-oxo-3,4-dihydropyrido[2,3-b]pyrazin-8-yl)oxy)phenyl)urea; and
1-(3-(tert-butyl)-1-phenyl-1H-pyrazol-5-yl)-3-(2,5-difluoro-4-((3-oxo-3,4-dihydropyrido[2,3-b]pyrazin-8-yl)oxy)phenyl)urea;

5)
1-(3-(tert-butyl)-1-(4-methoxyphenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(3-chlorophenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(3-methoxyphenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(3-fluorophenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(quinolin-6-yl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(4-cyanophenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(3,4-dimethylphenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(4-(2-(piperidin-1-yl)ethoxy)phenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(4-(2-morpholinoethoxy)phenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(3-chloro-4-methylphenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(3-chloro-4-fluorophenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(4-(3-(dimethylamino)propoxy)phenyl)-1H-pyrazol-5-yl)-3-(2-(methylthio)-4-((3-oxo-3,4-dihydropyrido[2,3-b]pyrazin-8-yl)oxy)phenyl)urea; and
1-(3-(tert-butyl)-1-(4-(morpholinomethyl)phenyl)-1H-pyrazol-5-yl)-3-(2-(methylthio)-4-((3-oxo-3,4-dihydropyrido[2,3-b]pyrazin-8-yl)oxy)phenyl)urea;

6)
1-(3-(tert-butyl)-1-(quinolin-6-yl)-1H-pyrazol-5-yl)-3-(2-fluoro-4-((3-oxo-3,4-dihydropyrido[2,3-b]pyrazin-8-yl)oxy)phenyl)urea;
1-(3-(tert-butyl)-1-(quinolin-6-yl)-1H-pyrazol-5-yl)-3-(4-((3-oxo-3,4-dihydropyrido[2,3-b]pyrazin-8-yl)oxy)naphthalen-1-yl)urea; and
1-(3-(tert-butyl)-1-(quinolin-6-yl)-1H-pyrazol-5-yl)-3-(2-(methylthio)-4-((3-oxo-3,4-dihydropyrido[2,3-b]pyrazin-8-yl)oxy)phenyl)urea;

7)
5-(4-(3-(3-(tert-butyl)-1-phenyl-1H-pyrazol-5-yl)ureido)-3-(methylthio)phenoxy)-N-methylnicotinamide;
N-(4-(4-(3-(3-(tert-butyl)-1-phenyl-1H-pyrazol-5-yl)ureido)-3-(methylthio)phenoxy)pyridin-2-yl)acetamide;
tert-butyl (4-(4-(3-(3-(tert-butyl)-1-phenyl-1H-pyrazol-5-yl)ureido)-3-(methylthio)phenoxy)pyridin-2-yl)carbamate;
tert-butyl (2-((4-(4-(3-(3-(tert-butyl)-1-phenyl-1H-pyrazol-5-yl)ureido)-3-(methylthio)phenoxy)pyridin-2-yl)amino)-2-oxoethyl)(methyl)carbamate;
N-(4-(4-(3-(3-(tert-butyl)-1-phenyl-1H-pyrazol-5-yl)ureido)-3-(methylthio)phenoxy)pyridin-2-yl)-2-(methylamino)acetamide;
1-(3-(tert-butyl)-1-phenyl-1H-pyrazol-5-yl)-3-(4-((2-((2-(dimethylamino)ethyl)amino)pyridin-4-yl)oxy)-2-(methylthio)phenyl)urea hydrochloride;
ethyl (4-(4-(3-(3-(tert-butyl)-1-phenyl-1H-pyrazol-5-yl)ureido)-3-(methylthio)phenoxy)pyridin-2-yl)carbamate;
benzyl (4-(4-(3-(3-(tert-butyl)-1-phenyl-1H-pyrazol-5-yl)ureido)-3-(methylthio)phenoxy)pyridin-2-yl)carbamate;
ethyl (4-(4-(3-(3-(tert-butyl)isoxazol-5-yl)ureido)-3-(methylthio)phenoxy)pyridin-2-yl)carbamate;
ethyl (4-(4-(3-(5-(tert-butyl)thiazol-2-yl)ureido)-3-(methylthio)phenoxy)pyridin-2-yl)carbamate; and
ethyl (4-(4-(3-(5-(tert-butyl)isoxazol-3-yl)ureido)-3-(methylthio)phenoxy)pyridin-2-yl)carbamate;

8)
N-(4-(4-(3-(3-(tert-butyl)-1-phenyl-1H-pyrazol-5-yl)ureido)-3-fluorophenoxy)pyridin-2-yl)acetamide;
N-(4-(4-(3-(3-(tert-butyl)-1-phenyl-1H-pyrazol-5-yl)ureido)-3-(methylthio)phenoxy)pyridin-2-yl)acetamide;
tert-butyl (4-(4-(3-(3-(tert-butyl)-1-phenyl-1H-pyrazol-5-yl)ureido)-3-(methylthio)phenoxy)pyridin-2-yl)carbamate;
tert-butyl (2-((4-(4-(3-(3-(tert-butyl)-1-phenyl-1H-pyrazol-5-yl)ureido)-3-(methylthio)phenoxy)pyridin-2-yl)amino)-2-oxoethyl)(methyl)carbamate;
ethyl (4-(4-(3-(3-(tert-butyl)-1-phenyl-1H-pyrazol-5-yl)ureido)-3-(methylthio)phenoxy)pyridin-2-yl)carbamate;
ethyl (4-((4-(3-(3-(tert-butyl)-1-phenyl-1H-pyrazol-5-yl)ureido)naphthalen-1-yl)oxy)pyridin-2-yl)carbamate;
ethyl (4-((4-(3-(3-(tert-butyl)isoxazol-5-yl)ureido)naphthalen-1-yl)oxy)pyridin-2-yl)carbamate;
ethyl (4-((4-(3-(5-(tert-butyl)thiazol-2-yl)ureido)naphthalen-1-yl)oxy)pyridin-2-yl)carbamate;
ethyl (4-(4-(3-(3-(tert-butyl)isoxazol-5-yl)ureido)-3-(methylthio)phenoxy)pyridin-2-yl)carbamate;
ethyl (4-(4-(3-(5-(tert-butyl)thiazol-2-yl)ureido)-3-(methylthio)phenoxy)pyridin-2-yl)carbamate;
benzyl (4-(4-(3-(3-(tert-butyl)-1-phenyl-1H-pyrazol-5-yl)ureido)-3-(methylthio)phenoxy)pyridin-2-yl)carbamate;
N-(4-(4-(3-(3-(tert-butyl)-1-phenyl-1H-pyrazol-5-yl)ureido)-3-(methylthio)phenoxy)pyridin-2-yl)-2-hydroxyacetamide; and
tert-butyl (4-(4-(3-(3-(tert-butyl)-1-phenyl-1H-pyrazol-5-yl)ureido)-3-(methylthio)phenoxy)pyridin-2-yl)(2-(dimethylamino)ethyl)carbamate.

In a specific aspect is provided a compound, preferably for use as a medicament, more preferably for use in treating, ameliorating, delaying, curing and/or preventing a disease or condition associated with muscle cells and/or satellite cells, or a pharmaceutically acceptable salt thereof, wherein said compound is represented by a structure selected from the group consisting of: (aa), (ab), (ac), (ad), (ae), (ai), preferably (ab), (ae) and (ai), more preferably (ab) or (ai).

The “pharmaceutically salts” include inorganic as well as organic acids salts. Representative examples of suitable inorganic acids include hydrochloric, hydrobromic, hydroiodic, phosphoric, and the like. Representative examples of suitable organic acids include formic, acetic, trichloroacetic, trifluoroacetic, propionic, benzoic, cinnamic, citric, fumaric, maleic, methanesulfonic and the like. Further examples of pharmaceutically inorganic or organic acid addition salts include the pharmaceutically salts listed in J. Pharm. Sci. 1977, 66, 2, and in Handbook of Pharmaceutical Salts: Properties, Selection, and Use edited by P. Heinrich Stahl and Camille G. Wermuth 2002. In a preferred embodiment, the salt is selected from the group consisting of maleate, chlorhydrate, bromohydrate, and methanesulfonate. The “pharmaceutically salts” also include inorganic as well as organic base salts. Representative examples of suitable inorganic bases include sodium or potassium salt, an alkaline earth metal salt, such as a calcium or magnesium salt, or an ammonium salt. Representative examples of suitable salts with an organic base includes for instance a salt with methylamine, dimethylamine, trimethylamine, piperidine, morpholine or tris-(2-hydroxyethyl)amine. In a preferred embodiment, the salt is selected from the group consisting of sodium and potassium salt. As used herein, the terms “treatment”, “treat” or “treating” refer to any act intended to ameliorate the health status of patients such as treating, ameliorating, delaying, curing and/or preventing a disease or condition associated with muscle cells and/or satellite cells. In certain embodiments, such terms refer to the amelioration or eradication of the disease, or symptoms associated with it. In other embodiments, this term refers to minimizing the spread or worsening of the disease, resulting from the administration of one or more therapeutic agents to a subject with such a disease.

As used herein, the terms “subject”, “individual” or “patient” are interchangeable and refer to an animal, preferably to a mammal, even more preferably to a human, including adult and child. However, the term “subject” can also refer to non-human animals, in particular mammals such as dogs, cats, horses, cows, pigs, sheep and non-human primates, among others.

The terms “quantity,” “amount,” and “dose” are used interchangeably herein and may refer to an absolute quantification of a molecule.

As used herein, the terms “active principle”, “active ingredient” and “active pharmaceutical ingredient” are equivalent and refers to a component of a pharmaceutical composition having a therapeutic effect.

As used herein, the term “therapeutic effect” refers to an effect induced by an active ingredient, or a pharmaceutical composition according to the invention, capable to prevent or to delay the appearance or development of a disease or disorder, or to cure or to attenuate the effects of a disease or disorder.

As used herein, the term “effective amount” refers to a quantity of an active ingredient or of a pharmaceutical composition which prevents, removes or reduces the deleterious effects of the disease. It is obvious that the quantity to be administered can be adapted by the man skilled in the art according to the subject to be treated, to the nature of the disease, etc. In particular, doses and regimen of administration may be function of the nature, of the stage and of the severity of the disease to be treated, as well as of the weight, the age and the global health of the subject to be treated, as well as of the judgment of the doctor.

As used herein, the term “pharmaceutically acceptable excipient or carrier” refers to any ingredient except active ingredients that is present in a pharmaceutical composition. Its addition may be aimed to confer a particular consistency or other physical or gustative properties to the final product. An excipient or pharmaceutically acceptable carrier must be devoid of any interaction, in particular chemical, with the active ingredients.

The pharmaceutical or veterinary composition comprises a compound of the present invention and optionally a pharmaceutically acceptable excipient or carrier.

The compound according to the invention or the pharmaceutical composition according to the invention may be administered by any conventional route of administration. In particular, the compound or the pharmaceutical composition of the invention can be administered by a topical, enteral, oral, parenteral, intranasal, intravenous, intra-arterial, intramuscular, or subcutaneous administration and the like.

Preferably, the compound according to the invention or the pharmaceutical composition according to the invention is administered by enteral or parenteral route of administration. When administered parenterally, the compound according to the invention or the pharmaceutical composition according to the invention is preferably administered by intravenous route of administration. When administered enterally, the compound according to the invention or the pharmaceutical composition according to the invention is preferably administered by oral route of administration.

The pharmaceutical composition comprising the molecule is formulated in accordance with standard pharmaceutical practice (Lippincott Williams & Wilkins, 2000 and Encyclopedia of Pharmaceutical Technology, eds. J. Swarbrick and J. C. Boylan, 1988-1999, Marcel Dekker, New York) known by a person skilled in the art.

For oral administration, the composition can be formulated into conventional oral dosage forms such as tablets, capsules, powders, granules and liquid preparations such as syrups, elixirs, and concentrated drops. Nontoxic solid carriers or diluents may be used which include, for example, pharmaceutical grades of mannitol, lactose, starch, magnesium stearate, sodium saccharine, talcum, cellulose, glucose, sucrose, magnesium, carbonate, polysorbate (e.g., 20, 40, 65, 80, 100, 120), and the like. For compressed tablets, binders, which are agents which impart cohesive qualities to powdered materials, are also necessary. For example, starch, gelatin, sugars such as lactose or dextrose, and natural or synthetic gums can be used as binders. Disintegrants are also necessary in the tablets to facilitate break-up of the tablet. Disintegrants include starches, clays, celluloses, algins, gums and crosslinked polymers. Moreover, lubricants and glidants are also included in the tablets to prevent adhesion to the tablet material to surfaces in the manufacturing process and to improve the flow characteristics of the powder material during manufacture. Colloidal silicon dioxide is most commonly used as a glidant and compounds such as talc or stearic acids are most commonly used as lubricants. Detergents such as dimethyl sulfoxide, dimethyl acetamide and dimethylformamide could be added.

For transdermal administration, the composition can be formulated into ointment, cream or gel form and appropriate penetrants or detergents could be used to facilitate permeation, such as dimethyl sulfoxide, dimethyl acetamide and dimethylformamide.

For transmucosal administration, nasal sprays, rectal or vaginal suppositories can be used. The active compound can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate.

Pharmaceutical compositions according to the invention may be formulated to release the active drug substantially immediately upon administration or at any predetermined time or time period after administration.

The amount of compound according to the invention or of pharmaceutical composition according to the invention to be administered has to be determined by standard procedure well known by those of ordinary skills in the art. Physiological data of the patient (e.g. age, size, and weight) and the routes of administration have to be taken into account to determine the appropriate dosage, so as a therapeutically effective amount will be administered to the patient.

In a preferred embodiment, the total compound dose for each administration of the compound according to the invention or of the pharmaceutical composition according to the invention is comprised between 0.00001 and 1 g.

A compound according to the invention is a compound for use in treating, ameliorating, delaying, curing and/or preventing a disease or condition associated with muscle cells and/or satellite cells.

A muscle cell is a type of cell found in muscle tissue. Optionally, a muscle cell can be a striated muscle cell. Preferably, a muscle cell is a skeletal muscle cell. During myogenesis, muscle cells are formed from muscle progenitors, including Pax7 positive cells. Diseases or conditions associated with muscle cells include myopathies including congenital myopathies and acquired myopathies, sarcopenia and cachexia. Examples of congenital myopathies are (muscular) dystrophies such as Duchenne muscular dystrophy (DMD), Becker muscular dystrophy (BMD), congenital muscular dystrophy, myotonic muscular dystrophy, limb-girdle muscular dystrophy, facioscapulohumeral muscular dystrophy (FSHD) and LAMA2 and SEPN1 myopathies; and non-dystrophic congenital myopathies such as nemaline myopathy, spinal muscular atrophy, SMARD1, multi/minicore myopathy and centronuclear myopathy. Examples of acquired myopathies include myopathies induces by drugs, alcohol and toxic agents, dermatomyositis and polymyositis.

Preferably, a disease or a condition associated with a muscle cell and/or a satellite cell is not facioscapulohumeral muscular dystrophy (FSHD).

Satellite cells, also known as muscle stem cells, are small multipotent cells, present in muscle tissue, which are precursors of skeletal muscle cells. Although these cells are quiescent under normal physiological conditions, they are activated in response to trauma and therefore play an important role in muscle repair and regeneration. Hence, diseases or conditions associated with satellite cells are often diseases or conditions wherein muscle cells are unable to repair and/or regenerate. Optionally, diseases or conditions associated with satellite cells include aging. Important markers for satellite cells which are able to play role in muscle repair and regeneration are Pax3 and Pax7, preferably Pax7. Preferably, the presence of Pax7 is detected via immunostaining using the anti-Pax7 antibody from the Developmental Studies Hybridoma Bank (DSHB).

In diseases or conditions associated with muscle cells, said muscle cells may have a decreased functionality, integrity and/or survival (i.e. said muscle cells have an increased degeneration) compared to a corresponding muscle cell from a healthy individual. Functionality in this context refers to, amongst other parameters, the ability of the muscle cell to contract. Preferably, the satellite cells in the muscle tissue wherein said muscle cells are comprised have a decreased capability in muscle repair and regeneration compared to corresponding satellite cells in a healthy individual.

In diseases or conditions associated with satellite cells, said satellite cells have a decreased capability in muscle repair and regeneration compared to corresponding satellite cells in a healthy individual. Preferably, the muscle cells comprised in the muscle tissue comprising said satellite cells have a decreased functionality, integrity and/or survival (i.e. said muscle cells have an increased degeneration) compared to a corresponding muscle cell from a healthy individual. Functionality in this context refers to, amongst other parameters, the ability of the muscle cell to contract.

A compound according to the invention is a compound for use in treating, ameliorating, delaying, curing and/or preventing a disease or condition associated with muscle cells and/or satellite cells. Hence, the administration to a patient of a compound according to the invention is associated with the alleviation of one or more characteristics of said muscle cell, or of a muscle cell comprised in the muscle tissue comprising said satellite cell, from said patient, preferably with the improvement of muscle fiber functionality, integrity and/or survival. Moreover, the administration of said compound may increase the number of satellite cells, or the number of satellite cells comprised in the muscle tissue comprising said muscle cell, relative to the number before administration; and preferably also increases the capability of said satellite cell, or the satellite cells comprised in the muscle tissue comprising said muscle cell, in muscle repair and regeneration. The number of satellite cells is preferably determined by measuring the number of Pax7 positive cells. Said increase of the number of satellite cells is preferably at least 5%, more preferably 10%, even more preferably 20%, most preferably 30, 32, 34; 36, 38, 40, 42, 44, 46, 48 or 50%, relative to the number of satellite cells in the assay before administration of the compound. In a preferred embodiment, the increase of the number of satellite cells is assessed in vitro, more preferably in vitro in an assay as carried out in the experimental part in the myotube assay of examples 78 and 79. Said increase of the number of satellite cells in vitro is preferably at least 5%, more preferably 10%, even more preferably 20%, most preferably 30, 32, 34; 36, 38, 40, 42, 44, 46, 48 or 50%, relative to the number of satellite cells in the assay before administration of the compound

Hence, in a preferred embodiment is provided a compound according to the invention, wherein said compound is able to induce the generation of myotubes and/or increase the number of Pax7 positive cells in muscle tissue comprising said muscle cell and/or said satellite cell, relative to the number of Pax7 positive cells in said muscle tissue before administration of said compound. Preferably said increase in the number of Pax7 positive cells is at least 5%, more preferably said increase in the number of Pax7 positive cells is 10%, even more preferably said increase in the number of Pax7 positive cells is at least 20%, most preferably said increase in the number of Pax7 positive cells is at least 30, 32, 34; 36, 38, 40, 42, 44, 46, 48 or 50%.

An alleviation of one or more characteristics of a muscle cell from a patient may be assessed by a variety of assays on a myogenic cell or muscle cell from a patient. The following test results, obtained from an assay on a myogenic cell or muscle cell from a patient, correlate with improved characteristics of a muscle cell from said patient: reduced cytosolic calcium concentration in muscle cells, decreased collagen synthesis, modified morphology, altered lipid biosynthesis, decreased oxidative stress, and/or improved muscle fiber function, integrity, and/or survival. These parameters are usually assessed using immunofluorescence and/or histochemical analyses of cross sections of muscle biopsies or primary cultures of patient muscle cells.

The improvement of muscle fiber function, integrity and/or survival may be assessed using at least one of the following assays: a detectable decrease of creatine kinase in blood, a detectable decrease of necrosis of muscle fibers in a biopsy cross-section of a muscle suspected to be dystrophic, a detectable decrease of fibrosis in a muscle suspected to be dystrophic, a detectable decrease in fat accumulation in a muscle suspected to by dystrophic, and/or a detectable increase of the homogeneity of the diameter of muscle fibers in a biopsy cross-section of a muscle suspected to be dystrophic. Each of these assays is known to the skilled person.

Creatine kinase may be detected in blood as described in Hodgetts et al. (2006). A detectable decrease in creatine kinase may mean a decrease of 5%, 10%, 20%, 30%, 40%, 50%, 60%, 70%, 80%, 90% or more compared to the concentration of creatine kinase in blood before administration of a compound according to the invention.

A detectable decrease of necrosis of muscle fibers is preferably assessed in a muscle biopsy, more preferably as described in Hodgetts et al. (2006), using biopsy cross-sections. A detectable decrease of necrosis may be a decrease of 5%, 10%, 20%, 30%, 40%, 50%, 60%, 70%, 80%, 90% or more of the area wherein necrosis has been identified using biopsy cross-sections. The decrease is measured by comparison to the necrosis as assessed before administration of a compound according to the invention.

A detectable increase of the homogeneity of the diameter of a muscle fiber is preferably assessed in a muscle biopsy cross-section, more preferably as described in Hodgetts et al. (2006). The increase is measured by comparison to the homogeneity of the diameter of a muscle fiber before administration of a compound according to the invention.

An in vitro assay for assessing the efficacy of compounds for use in treating, ameliorating, delaying, curing and/or preventing a disease or condition associated with muscle cells and/or satellite cells is described in example 78. Herein, the generation of myotubes and satellite cells after adding a compound to a myotube assay workflow is assessed. Said myotube assay workflow is derived from wild-type or DMD human induced pluripotent stem cells (WT hiPSC or DMD hiPSC). Before the addition of said compound, the assay comprises satellite-like cells, but no significant amount of myotubes.

The efficacy of said compound for use in treating, ameliorating, delaying, curing and/or preventing a disease or condition associated with muscle cells and/or satellite cells is positively correlated with the generation of myotubes (i.e. the increase of the area occupied by myotubes on the assay) and/or satellite cells (i.e. the increase of the number of satellite cells) in the assay after adding said compound. The generation of myotubes by the addition of said compound is expressed as the EC₅₀ value, the concentration of said compound that corresponds with the half-maximal generation of myotubes, and the MA (normalized myotube area) value, the normalized area occupied by generated myotubes on the assay. The EC₅₀ value is determined using the GraphPad Prism software after fitting a 10 points dose-response 4 parameter logistic regression curve (ranging from 1.5 nM to 30 μM). Since muscle cells are formed from myotubes during myogenesis, the generation of myotubes in the assay is a good indicator of the ability of said compound to induce the formation of functional muscle cells in vivo. The number of satellite cells is measured as the percentage of Pax7 positive cells, relative to the total number of cells in the assay, as shown in FIG. 3. The percentage of Pax7 is a good indicator of the ability of said compound to induce the formation of satellite cells which are able to play a role in muscle repair and regeneration in vivo.

In example 2, it is shown that all tested compounds according to the invention are suitable for use in treating, ameliorating, delaying, curing and/or preventing a disease or condition associated with muscle cells and/or satellite cells.

The efficacy of a compound according to the invention for use in treating, ameliorating, delaying, curing and/or preventing a disease or condition associated with muscle cells and/or satellite cells is preferably assessed by the myotube assay workflow described above and in Example 2.

In a preferred embodiment is provided a compound according to the invention for use in treating, ameliorating, delaying, curing and/or preventing a disease or condition associated with muscle cells and/or satellite cells, wherein said compound is able to induce the generation of myotubes and/or Pax7 positive cells in muscle tissue comprising said muscle cells and/or said satellite cells, preferably assessed by the myotube assay workflow described above and in Example 2. In an even more preferred embodiment, said disease or condition is not facioscapulohumeral muscular dystrophy (FSHD).

In a preferred embodiment is provided a compound according to the invention for use in treating, ameliorating, delaying, curing and/or preventing a disease or condition selected from the group consisting of sarcopenia, cachexia, Duchenne muscular dystrophy and Becker muscular dystrophy, preferably wherein said compound is able to induce the generation of myotubes and Pax7 positive cells in muscle tissue, preferably assessed by the myotube assay workflow described above and in Example 2.

In a preferred embodiment is provided a compound according to the invention for use in treating, ameliorating, delaying, curing and/or preventing a disease or condition selected from the group consisting of Duchenne muscular dystrophy and Becker muscular dystrophy, preferably wherein said compound is able to induce the generation of myotubes and Pax7 positive cells in muscle tissue, preferably assessed by the myotube assay workflow described above and in Example 2.

In a preferred embodiment is provided a compound according to the invention for use in treating, ameliorating, delaying, curing and/or preventing a disease or condition selected from the group consisting of sarcopenia and cachexia, preferably wherein said compound is able to induce the generation of myotubes and Pax7 positive cells in muscle tissue, preferably assessed by the myotube assay workflow described above and in Example 2.

Duchenne muscular dystrophy is a severe type of muscular dystrophy caused by a mutation in the dystrophin gene, resulting in muscle cells with a reduced biological functionality, leading to progressive muscle weakness. Becker muscular dystrophy is a similar disorder, albeit with less severe symptoms. Sarcopenia is the loss of skeletal muscle mass due to aging. Cachexia, in the context of this application, is the loss of skeletal muscle mass associated with a chronic illness such as cancer, acquired immune deficiency syndrome (AIDS) or chronic obstructive pulmonary disease (COPD).

Duchenne muscular dystrophy and Becker muscular dystrophy are diseases associated with muscle cells and satellite cells. Without being bound to this theory, satellite cells comprised in muscle tissue of a patient suffering from DMD or BMD are not able to undergo asymmetric division, and are therefore not able to play a role in the regeneration and/or repair of said muscle, due to lack of a functional dystrophin in said satellite cells (Chang et al., 2016, Trends Mol Med., 22(6): 479-496). In individuals not suffering from DMD or BMD, said satellite cells are able to undergo asymmetric division, eventually leading to a new satellite cell and a myotube (Chang et al., 2016, Trends Mol Med., 22(6): 479-496). Additionally or alternatively, without being bound to this theory, the number of satellite cells in muscle tissue of a patient suffering from DMD or BMD is significantly decreased relative to muscle tissue of individuals not suffering from DMD or BMD.

Alleviating one or more symptom(s) of Duchenne muscular dystrophy and/or Becker muscular dystrophy in an individual using a compound according to the invention may be assessed by any of the following assays: prolongation of time to loss of walking, improvement of muscle strength, improvement of the ability to lift weight, improvement of the time taken to rise from the floor, improvement in the 6-minute walk test (6MWT), improvement in the time taken for four-stairs climbing, improvement of the leg function grade, improvement of the pulmonary function, improvement of cardiac function, improvement of the quality of life. Each of these assays is known to the skilled person. As an example, the publication of Manzur et al. (2008), gives an extensive explanation of each of these assays. For each of these assays, as soon as a detectable improvement or prolongation of a parameter measured in an assay has been found, it will preferably mean that one or more symptoms of Duchenne and/or Becker muscular dystrophy has been alleviated in an individual using a compound according to the invention. Detectable improvement or prolongation is preferably a statistically significant improvement or prolongation as described in Hodgetts et al. (2006). Alternatively, the alleviation of one or more symptom(s) of Duchenne and/or Becker muscular dystrophy may be assessed by measuring an improvement of a muscle fiber function, integrity and/or survival. In a preferred method, one or more symptom(s) of a DMD and/or BMD is/are alleviated and/or one or more characteristic(s) of one or more muscle cells from a DMD and/or BMD is/are improved. Such symptoms or characteristics may be assessed at the cellular, tissue level or on the patient self.

Alleviating one or more symptom(s) of sarcopenia and/or cachexia in an individual using a compound according to the invention may be assessed by any of the following assays: prolongation of time to loss of walking, improvement of muscle strength, improvement of the ability to lift weight, improvement of the time taken to rise from the floor, improvement in the ten-meter walking time, improvement in the time taken for four-stairs climbing, improvement of the leg function grade, improvement of the quality of life. For each of these assays, as soon as a detectable improvement or prolongation of a parameter measured in an assay has been found, it will preferably mean that one or more symptoms of sarcopenia and/or cachexia has been alleviated in an individual using a compound according to the invention. Alternatively, the alleviation of one or more symptom(s) of sarcopenia and/or cachexia may be assessed by measuring an improvement of a muscle fiber function, integrity and/or survival. In a preferred method, one or more symptom(s) of sarcopenia and/or cachexia is/are alleviated and/or one or more characteristic(s) of one or more muscle cells from sarcopenia is/are improved. Such symptoms or characteristics may be assessed at the cellular, tissue level or on the patient self.

### EXAMPLES

The following examples are offered for illustrative purposes only, and are not intended to limit the scope of the present invention in any way.

### Example 1: Synthesis of the Compounds

### General Synthetic Protocol

The reagents and starting materials are commercially available and/or, using well-known techniques, can be readily synthesized by one of ordinary skill in the art. Unless otherwise noted, all commercially starting materials were used without further purification.

The following abbreviation may be used in the examples and throughout the specification: g (grams), mg (milligrams), L (liter), mL (milliliters), μL (microliters), M (molar), % (percentage), MHz (megahertz), mmol (millimoles), min (minutes), AcOEt or EtOAc or EA (ethyl acetate), Et₂O (diethyl ether), % (percent), DCM (dichloromethane), DMSO (dimethyl sulfoxide), NEt₃ or TEA (triethylamine), TBDMS-Cl (tert-butyldimethylsilyl chloride), DMF (dimethylformamide), EtOH (ethyl alcohol), Pd/C (palladium on carbon), H₂ (hydrogen gas), NaN₃ (sodium azide), DIPEA (diisopropyl ethyl amine), NaH (sodium hydride), Na₂CO₃ (sodium carbonate), NaHCO₃ (sodium hydrogencarbonate), MeONa (sodium methoxide), CBr₄ (carbon tetrabromide), PPh₃ (triphenylphosphine), CO₂ (carbon dioxide), DBU (1,8-Diazabicyclo[5.4.0]undec-7-ene), TFA (trifluoroacetic acid), MeCN or CH₃CN (acetonitrile), K₂CO₃ (potassium carbonate), NMP (1-methyl-2-pyrrolidinone), CHCl₃ (chloroform), CuI (copper iodide), t-BuOK (potassium tert-butoxide), Cs₂CO₃ (cesium carbonate), CuCN (copper cyanide), i-AmONO (isoamyl nitrite), SnCl₂.2H₂O (stannous chloride dihydrate), Ac₂O (acetic anhydride), N₂H₄.H₂O (hydrazine monohydrate), PE (petroleum ether), NEt₃.3HF (triethylamine trifluoride), p-TSA (p-toluenesulfonic acid), NH₃ (ammoniac), Me₂CO (acetone), SOCl₂ (thionyl chloride), DCE (dichloroethane), DMAP (N,N-dimethylaminopyridine), (Boc)₂O (Di-tert-butyl dicarbonate), ° C. (Celsius degrees), MeOH (methanol), Hz (Hertz), LCMS (Liquid Chromatography Mass Spectrum), MS (Mass Spectrum), ES (Electrospray), HPLC (High Pressure Liquid Chromatography), NMR (Nuclear Magnetic Reasonance), 1H (proton), MgSO₄ (magnesium sulphate), R.T. (Room Temperature), KOH (potassium hydroxide), NaOH (sodium hydroxide), h (hour), HCl (hydrochloric acid), THF (tetrahydrofuran), N₂ (nitrogen), eq (equivalent), mm (millimeters), s (singulet), d (doublet), t (triplet), m (multiplet), q (quintuplet), bs (broad singulet), dd (doublet of doublet), dt (doublet of triplet), td (triplet of doublet), dq (doublet of quintuplet).

NMR spectra were registered on a 400 MHz Bruker DPX and processed using Bruker XWinNMR software. All commercially obtained reagents were used without further purification.

All references to brine refer to a saturated aqueous solution of NaCl. Unless otherwise indicated, all temperatures are expressed in ° C. All reactions are not conducted under an inert atmosphere at room temperature unless otherwise noted.

### Synthesis of Compounds According to the Invention

Compounds represented by structure (I) may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley et Sons). These groups are removed at a convenient stage of the compound synthesis using methods that are readily apparent to those skilled in the art. The selection of process as well as the reaction conditions and order of their execution shall be consistent with the preparation of compounds of structure (I).

The compound of structure (I) may be represented as a mixture of enantiomers, which may be resolved into the individual pure R- or S-enantiomers. If for instance, a particular enantiomer of the compound of structure (I) is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group such as amino, or an acidic functional group such as carboxyl, this resolution may be conveniently performed by fractional crystallization from various solvents, of the salts of the compounds of structure (I) with optical active acid or by other methods known in the literature, e.g. chiral column chromatography.

Resolution of the final product, an intermediate or a starting material may be performed by any suitable method known in the art as described by E. L. Eliel, S. H. Wilen and L. N. Mander (1984) Stereochemistry of Organic Compounds, Wiley-Interscience.

All the products generated from the following reactions can be isolated and purified employing standard techniques, such as extraction, chromatography, crystallization, distillation, and the like.

General routes to prepare the Examples of the present invention are shown in the Schemes and examples that follow.

All substituents in the synthetic Schemes unless otherwise indicated, are as previously defined.

The compounds of structure (I) may be prepared by general route of synthesis as disclosed in the following methods.

The compounds of structure (I) wherein A, B, L and —C are as described above may be prepared following the Scheme 1. The amine 1.1 react with diphosgene or triphosgene to obtain the corresponding isocyanate 1.2 (Liu, Dazhi & al, Bioorganic & Medicinal Chemistry, 2013, 21(11), 2960-2967; Snorri Th. Sigurdsson & al, J. Org. Chem., 1996, 61, 3883-3884). Then 1.2 can react with multiple aryl or heteroaryl amine 1.3 to generate the final compound 1.4 by reaction in DMF or dioxane (WO2009034308).

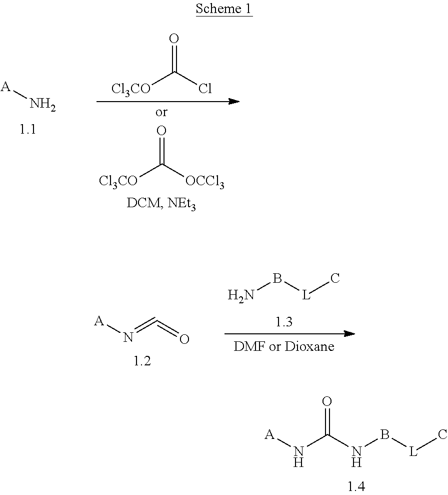

A similar procedure as those described in Scheme 1 can be used starting from commercially available amine 1.1. Following the procedure described in Pavia, Michael & al, Journal of Medicinal Chemistry, 1990, 33(2), 854-861, 1.6 is obtained after deprotection of 1.5 in presence of triethylamine trihydrofluoride (WO2001029025). Then the phenol 1.6 can give compound 1.7 in presence of the chloropyridine to be subsequently reduced to the compound 1.8 (Menard, Delphine & al, Journal of Medicinal Chemistry, 2009, 52(13), 3881-3891). In a presence of ethyl oxoacetate compound (Zambon, Alfonso & al, Journal of Medicinal Chemistry, 2010, 53(15), 5639-5655) give final derivative 1.9 and in presence of triphosgene the final derivative 1.15 (Menard, Delphine & al, Journal of Medicinal Chemistry, 2009, 52(13), 3881-3891).

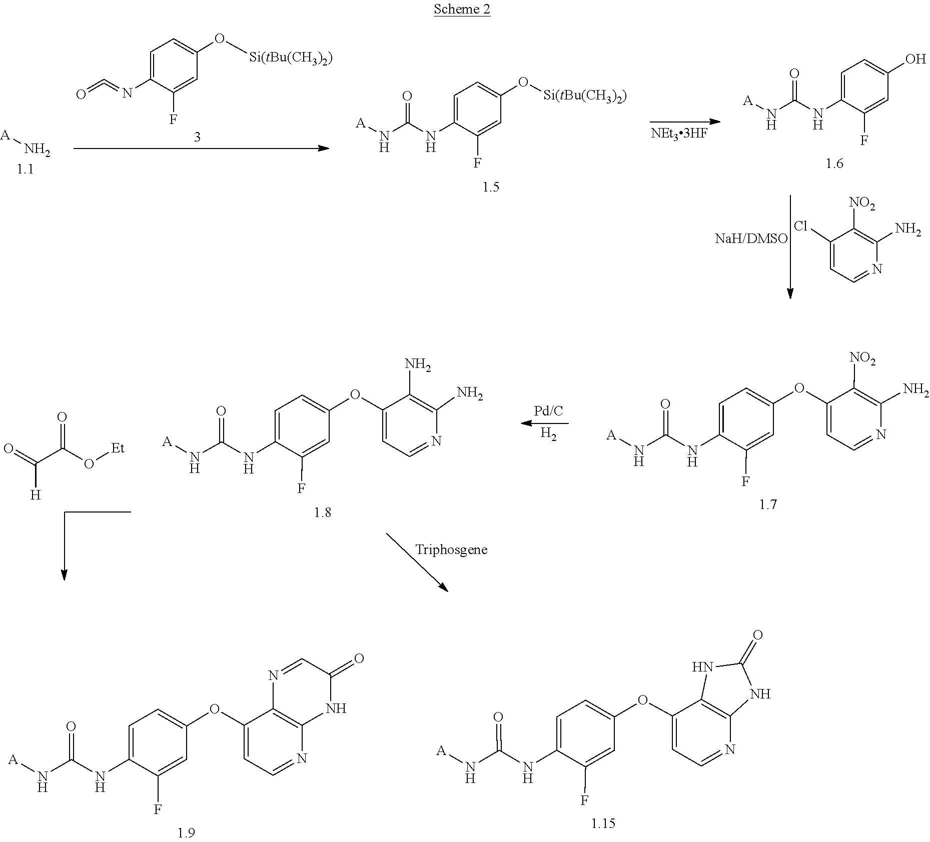

Alternatively, the aryl or heteroarylamine 1.3 can be first transformed in isocyanate 1.10 by reaction with diphosgene or triphosgene (Kurita, Keissuke & al, Journal of Organic Chemistry, 1976, 41(11), 2070-2071, Baraldi, Pier Giovanni & al, Journal of Medicinal Chemistry, 2001, 44(17), 2735-2742 or Liu, Dazhi & al, Bioorganic & Medicinal Chemistry, 2013, 21(11), 2960-2967). The corresponding isocyanate 1.10 react then with the amine 1.1 (Pavia, Michael & al, Journal of Medicinal Chemistry, 1990, 33(2), 854-861) to afford 1.4 compounds of structure (I) (Scheme 3).

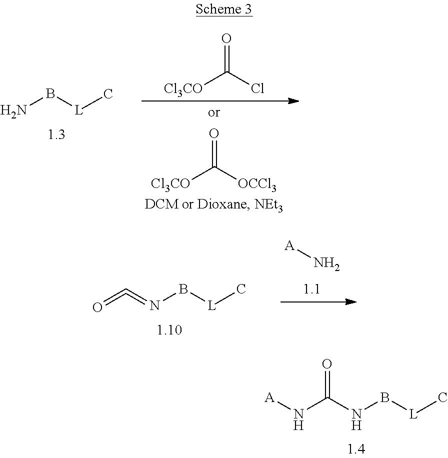

Yet another approach to the compounds of structure (I) can be represented Scheme 4 with the formation of the carbamate 1.11 from the amine 1.1 following the procedure described in Hron, Rebecca J. & al, Bioorganic & Medicinal Chemistry, 2016, 24(23), 6183-6193 or the procedure described in Kono, Mitsunori & al, Bioorganic & Medicinal Chemistry, 2013, 21(1), 28-41. To achieve this procedure the intermediate 1.11 react with the aryl or heteroarylamine 1.3 to get the final compound 1.4 (WO2018132372).

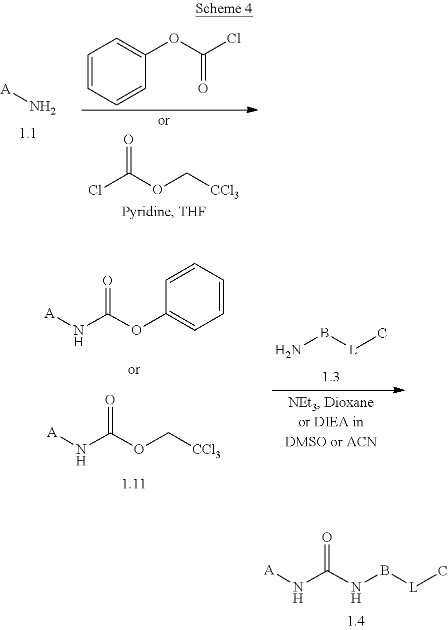

Yet another approach to the compound of structure (I) is described in Scheme 4 in particular for those compounds of structure (I) for which the formation of the acyl azide was particularly indicated. For this particular synthesis the reaction starts with a carboxylic acid 1.12 which is transformed in carbamoyl chloride 1.13 in a presence of oxalyl chloride (Zhou, Yuan & al, Bioorganic & Medicinal Chemistry, 2018, 26(1), 84-95). In the presence of sodium azide the intermediate 1.13 gives the acyl azide 1.14 (Sagandira, Cloudius R. and Watts, Paul, European Journal of Organic Chemistry, 2017, 2017(44), 6554-6565) which react in DMSO with the aryl or heteroarylamine 1.3 to obtain the final compound 1.4 (Feng, Peng & al, Organic Letters, 2014, 16(12), 3388-3391)

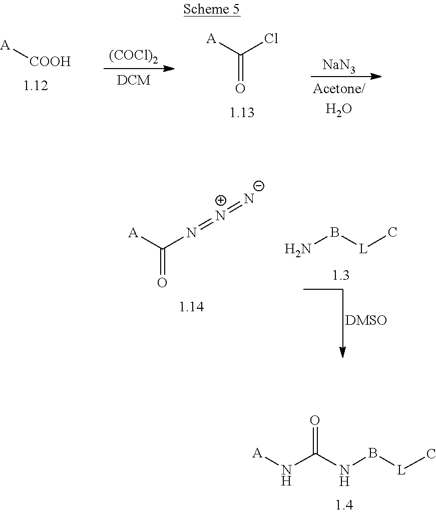

The reagents and starting materials are commercially available and/or, using well-known techniques, can be readily synthesized by one of ordinary skill in the art. Unless otherwise noted, all commercially starting materials were used without further purification.

### Synthesis of Key Intermediates

### Synthesis of 3: tert-butyl(3-fluoro-4-isocyanatophenoxy)dimethylsilane

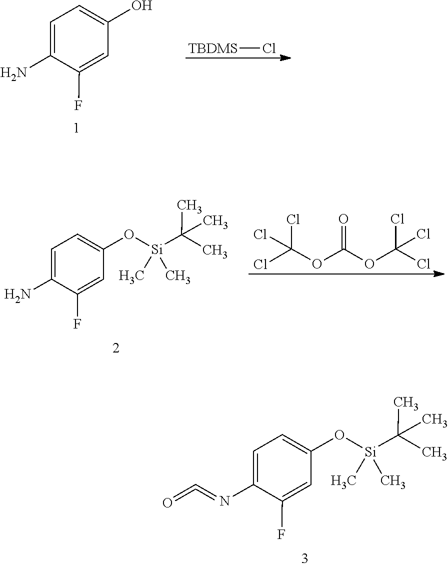

### Step 1. Synthesis of Compound (2)

TBDMS-Cl (14.23 g, 93.6 mmol, 1.2 eq) was added to a solution of 1 (10.0 g, 78.6 mmol, 1 eq) and imidazole (6.42 g, 93.6 mmol, 1.2 eq) in THF (100 mL) and the reaction mixture was stirred at ambient temperature for 12 h. The formed precipitate was filtered, the solvent was evaporated under reduced pressure, the residue was purified by column chromatography on silica gel (DCM). Yield of compound 2: 95%, 18 g.

### Step 2. Synthesis of Compound (3): tert-butyl(3-fluoro-4-isocyanatophenoxy)dimethylsilane

Triphosgene (5.77 g, 19.5 mmol, 1 eq) was added portions to a solution of compound 2 (4.7 g, 19.5 mmol, 1 eq) and Et₃N (8.4 mL, 58.5 mmol, 3 eq) in DCM (50 mL) at −20° C., the reaction mixture was stirred at ambient temperature overnight. Solvent was removed under reduced pressure, THF (50 mL) was added to the residue, the formed precipitate was filtered off, solvent was evaporated under reduced pressure to give compound 3. The residue was used directly to the next step without additional purification. Yield of compound 3: 99%, 5.2 g.

### Synthesis of 8: 8-(4-amino-3-fluorophenoxy)pyrido[2,3-b]pyrazin-3(4H)-one

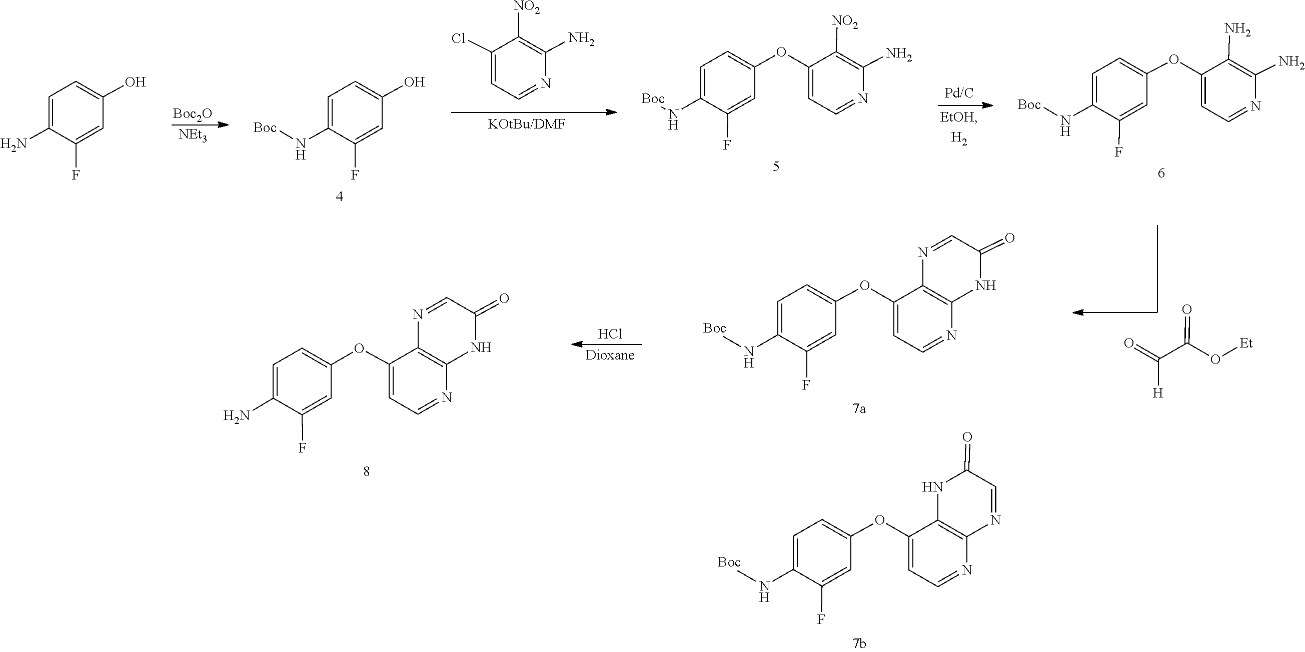

### Step 1. Synthesis of Compounds (4)

(Boc)₂O (34.0 g, 0.15 mol) was added by portions to a solution of compound 1 (20.0 g, 0.15 mol) and Et₃N (22.0 mL, 0.15 mol) in DCM (200 mL). The reaction mixture was stirred at ambient temperature for 2 h and evaporated to dryness. The residue was purified by flash chromatography on silica gel with DCM to afford compound 4 (Yield: 56%, 20 g).

### Step 2. Synthesis of Compounds (5)

Potassium tert-butoxide (8.4 g, 0.075 mol) was added portion wise to a solution of compound 4 (13.0 g, 0.057 mol) in dry DMF (100 mL) and stirred at ambient temperature 30 min. 4-Chloro-3-nitropyridin-2-amine (10.0 g, 0.057 mmol) was added as a solid in one portion and the reaction mixture was subsequently heated at 85° C. for 1 hours. The reaction mixture was cooled, diluted with water (200 mL) and formed precipitate was filtered off, washed with water, hexane and dried to afford compound 5 (Yield: 83%, 17.5 g).

### Step 3. Synthesis of Compounds (6)

10% Pd/C (2.0 g, ˜10% weight) were added to a solution of compound 5 (17.5 g, 0.05 mol) in mixture EtOH/EtOAc (200 mL, 2:1). Hydrogen was passed through the reaction mixture under stirring at room temperature for 16 h. Then the catalyst was filtered off through Celite. The filtrate was evaporated under reduced pressure to afford of compound 6 (Yield: 95%, 15 g).

### Step 4. Synthesis of Compounds (7a and 7b)

To a solution of compound 6 (15.0 g, 0.044 mol) in EtOH (200 mL) was added ethyl glyoxalate 50% solution in toluene (14 mL, 0.067 mol). Then reaction mixture stirred at ambient temperature for 16 h. The solvent was evaporated. The residue after evaporation was subjected to column chromatography on silica gel eluting with dichloromethane/ethyl acetate (0→100%) to afford the compound 7a (Yield: 33%, 5.5 g) and compound 7b (Yield: 60%, 10.0 g).

### Synthesis of Compounds (8): 8-(4-amino-3-fluorophenoxy)pyrido[2,3-b]pyrazin-3(4H)-one

HCl/dioxane (15 mL, 0.04 mol, 3M) was added to compound 7a (5.5 g, 0.014 mol). The reaction mixture stirred at ambient temperature 2 h, then evaporated under reduced pressure, then dichloromethane (100 mL) and saturated aqueous solution of potassium dicarbonate (100 mL) was added. The organic layer was separated, washed with brine, dried over sodium sulfate, filtered and concentrated to afford the compound 8 (Yield: 75%, 3 g) as a brown solid.

### Synthesis of 11: tert-butyl(4-isocyanatophenoxy)dimethylsilane

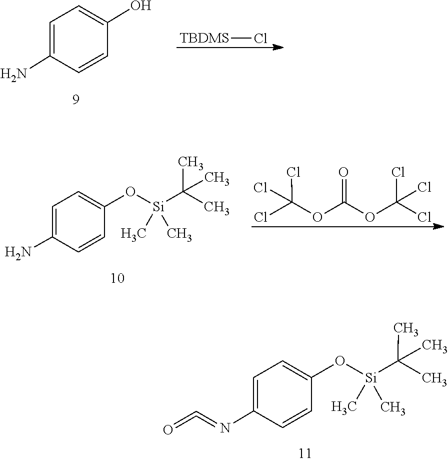

### Step 1. Synthesis of Compound (10)

TBDMS-Cl (14.23 g, 93.6 mmol, 1.2 eq) was added to a solution of 9 (8.57 g, 78.6 mmol, 1 eq) and imidazole (6.42 g, 93.6 mmol, 1.2 eq) in THF (100 mL) and the reaction mixture was stirred at ambient temperature for 12 h. The formed precipitate was filtered, the solvent was evaporated under reduced pressure, the residue was purified by column chromatography on silica gel (DCM) to obtain 10 (Yield: 97%, 17 g).

### Step 2. Synthesis of Compound (11): tert-butyl(4-isocyanatophenoxy)dimethylsilane

Triphosgene (5.77 g, 19.5 mmol, 1 eq) was added portions to a solution of compound 10 (4.35 g, 19.5 mmol, 1 eq) and Et₃N (8.4 mL, 58.5 mmol, 3 eq) in DCM (50 mL) at −20° C., the reaction mixture was stirred at ambient temperature overnight. Solvent was removed under reduced pressure, THF (50 mL) was added to the residue, the formed precipitate was filtered off, solvent was evaporated under reduced pressure to give compound 11. The residue was used directly to the next step without additional purification. Yield of compound 11: 97%, 4.73 g.

### Synthesis of 14: 1-phenyl-3-(1-(trifluoromethyl)cyclopropyl)-1H-pyrazol-5-amine

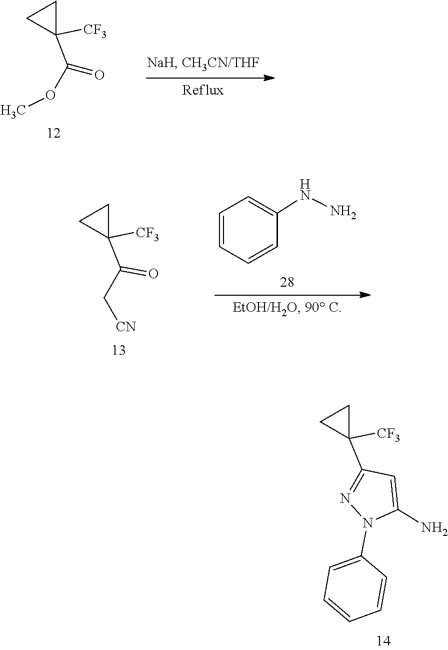 [SMILES: COC(=O)C1(C(F)(F)F)CC1 ; N#CCC(=O)C1(C(F)(F)F)CC1 ; NNc1ccccc1 ; Nc1cc(C2(C(F)(F)F)CC2)nn1-c1ccccc1]

### Step 1. Synthesis of Compounds (13)

To a slurry of NaH (1.0 g, 26.0 mmol) in THF (20 mL) heated at reflux, then was added dropwise over 10 minutes a solution compound 12 (2.0 g, 17.5 mmol) in acetonitrile (0 g, 19.0 mmol) and the reaction mixture heated at reflux overnight. After cooling to room temperature, the reaction was partitioned between ether and water, the aqueous layer acidified with 1N HCl(aq), the aqueous layer was extracted with Et₂O (2×50 mL), the combined organic layers were dried under Na₂SO₄, and evaporated to dryness. The residue was purified by flash chromatography on silica gel with hexane/ethyl acetate (0→50%) as an eluent to afford compound 13 (Yield: 46%, 1 g).

### Step 2. Synthesis of Compounds (14): 1-phenyl-3-(1-(trifluoromethyl)cyclopropyl)-1H-pyrazol-5-amine

To solution of compound 13 (1 g, 8.1 mmol) in EtOH (5 mL) was added water (5 mL), 1N NaOH(aq) (8 mL), and phenylhydrazine hydrochloride 28 (1.2 g, 8.1 mmol) and the mixture heated at 90° C. overnight. After cooling to room temperature, the mixture was diluted with water, extracted with EtOAc, the combined organic layers were dried under Na₂SO₄, and evaporated to dryness. The residue was purified by flash chromatography on silica gel with DCM/ethyl acetate (0→10%) as an eluent to afford compound 14 (Yield: 18%, 0.4 g).

### Synthesis of 18: 3-cyclopropyl-1-phenyl-1H-pyrazole-5-carboxylic Acid

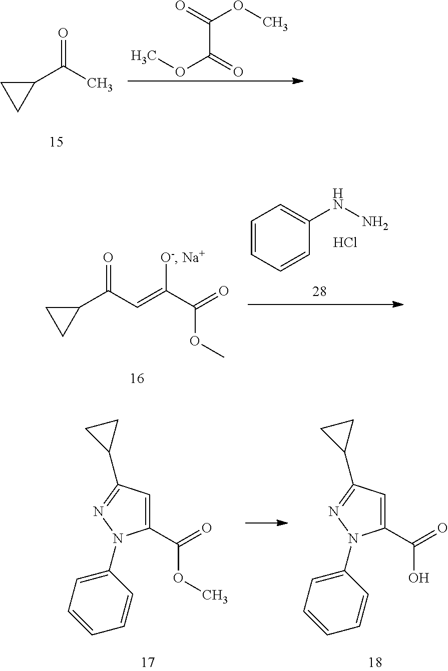

### Step 1. Synthesis of Compounds (17)

Compound 15 (5 g, 59 mmol), dimethyl oxalate (7 g, 59 mmol) and MeONa (3.3 g, 60 mmol) was mixed in MeOH (100 mL). After stirring for 2 h at 60° C., reaction mixture cooling to room temperature, the phenylhydrazine hydrochloride 28 (8.6 g, 59 mmol) was added to the reaction mixture. After stirring for 12 h at 60° C., reaction mixture cooling to room temperature. The solvent was then removed under reduced pressure. Water (200 mL) was added to the residue, and the resulting mixture was extracted with EtOAc (3×50 mL). The combined organic extracts were washed with brine and dried over Na₂SO₄. The solvent was then removed under reduced pressure. The crude product was purified with column using PE/EA as eluent to give compound 17 (Yield: 32%, 4.6 g).

### Step 2. Synthesis of Compounds (18): 3-cyclopropyl-1-phenyl-1H-pyrazole-5-carboxylic Acid

Compound 17 (4.6 g, 3.8 mmol) was dissolved in MeOH (50 mL) and H₂O (15 mL), and NaOH (4 g) were then added. After stirring for 12 h at 20° C., solvent was then removed under reduced pressure. Water (100 mL) was added to the residue, and conc. HCl were then added until reaching pH=3. The precipitate was filtered and dried under reduced pressure to get compound 18 (Yield: 78%, 3.4 g).

### Synthesis of 21: 3-(3-methyloxetan-3-yl)-1-phenyl-1H-pyrazol-5-amine

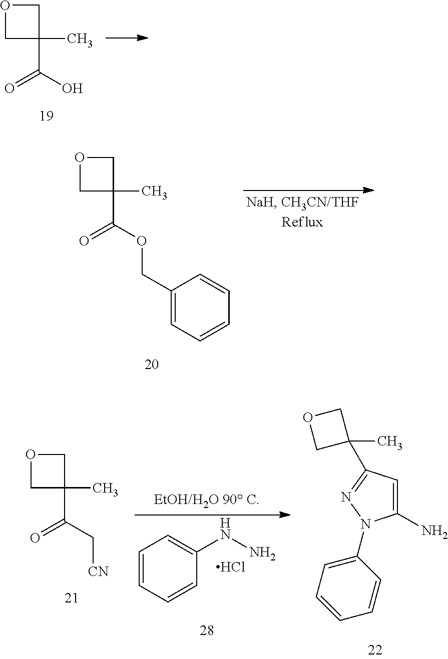

### Step 1. Synthesis of Compounds (20)

To a solution of compound 19 (2.44 g, 0.021 mol), DIPEA (3.8 mL, 0.021 mol) in DCE (50 mL) was added benzyl bromide (3.7 g, 0.021 mol). The reaction mixture was refluxed for 3 h. The solid was removed by filtration and the solution was evaporated to dryness to afford of compound 20 (Yield: 91%, 4 g).

### Step 2. Synthesis of Compounds (21)

To a slurry of NaH (0.6 g, 14.0 mmol) in THF (20 mL) heated at reflux, then was added dropwise over 10 minutes a solution compound 20 (2.0 g, 9.7 mmol) in acetonitrile (0.5 mL, 10.0 mmol) and the reaction mixture heated at reflux overnight. After cooling to room temperature, the reaction was partitioned between ether and water, the aqueous layer acidified with 1N HCl(aq), the aqueous layer was extracted with Et₂O (2×50 mL), the combined organic layers were dried under Na₂SO₄, and evaporated to dryness. The residue was purified by flash chromatography on silica gel with DCM/ethyl acetate (0→10%) as an eluent to afford compound 21 (Yield: 58%, 0.8 g).

### Step 3. Synthesis of Compounds (22): 3-(3-methyloxetan-3-yl)-1-phenyl-1H-pyrazol-5-amine

To solution of compound 21 (0.8 g, 5.6 mmol) in EtOH (5 mL) was added water (5 mL), 1N NaOH(aq) (5.7 mL), and phenylhydrazine hydrochloride 28 (0.8 g, 5.6 mmol) and the mixture heated at 90° C. overnight. After cooling to room temperature, the mixture was diluted with water, extracted with EtOAc, the combined organic layers were dried under Na₂SO₄, and evaporated to dryness. The residue was purified by flash chromatography on silica gel with DCM/ethyl acetate (0→10%) as an eluent to afford compound 22 (Yield: 52%, 0.67 g).

### Synthesis of 24: 1-(5-amino-3-(tert-butyl)-1H-pyrazol-1-yl)ethenone

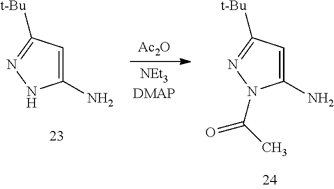

### Synthesis of Compounds (24): 1-(5-amino-3-(tert-butyl)-1H-pyrazol-1-yl)ethenone

The appropriate 3-tert-butyl-5-aminopyrazole 23 (2.0 mmol) was as dissolved in dichloromethane (2.5 mL). Triethylamine (2.2 mmol) and Ac₂O (2.0 mmol) were added followed by a catalytic amount of 4-dimethylaminopyridine (DMAP), and the mixture was stirred for 1 h at room temperature. Purification by flash chromatography furnished the corresponding 1-(5-amino-3-(tert-butyl)-1H-pyrazol-1-yl)ethenone 24 as a brown oil (Yield: 41%, 0.15 g).

### Synthesis of 26: 3-(tert-butyl)-1-(2-morpholinoethyl)-1H-pyrazol-5-amine

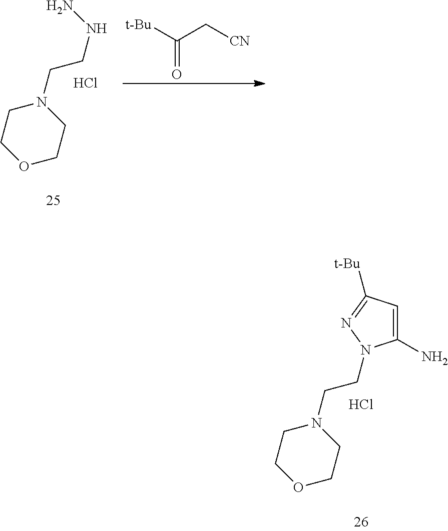 [SMILES: NNCCN1CCOCC1 ; Cl ; CC(C)(C)C(=O)CC#N ; CC(C)(C)c1cc(N)n(CCN2CCOCC2)n1 ; Cl]

### Synthesis of (26): 3-(tert-butyl)-1-(2-morpholinoethyl)-1H-pyrazol-5-amine

Suspension of hydrazine hydrochloride 25 (1 g, 3.9 mmol) and 4,4-dimethyl-3-oxopentanenitrile (0.49 g, 3.9 mmol) in 100 mL EtOH was refluxed for 16 h. After cooling the mixture was evaporated off then residue was purified by column chromatography on silica gel (DCM-MeOH) (Yield: 30%, 0.3 g).

### Synthesis of 29: phenyl (3-(tert-butyl)-1-(quinolin-6-yl)-1H-pyrazol-5-yl)carbamate

H.-G. Lee, M.-J. Kim, S.-E. Park, J.-J. Kim, S.-G. Lee, Y.-J. Yoon, Synlett, 2009, 2809-2814

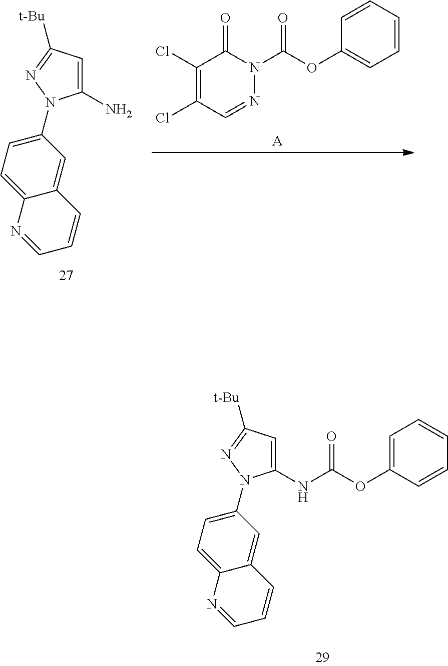

### Synthesis of (29): Phenyl (3-(tert-butyl)-1-(quinolin-6-yl)-1H-pyrazol-5-yl)carbamate

Mixture of compound 27 (0.21 g, 1.05 mmol) and compound A (0.31 g, 1.1 mmol) in 20 mL of anhydrous THF was refluxed overnight. Volatiles was evaporated and product 29 isolated by column chromatography on silica gel eluting DCM:EtOAc (9:1) (Yield: 49%, 0.2 g)

### Synthesis of 34: 3-(tert-butyl)-5-isocyanato-1-phenyl-1H-1,2,4-triazole

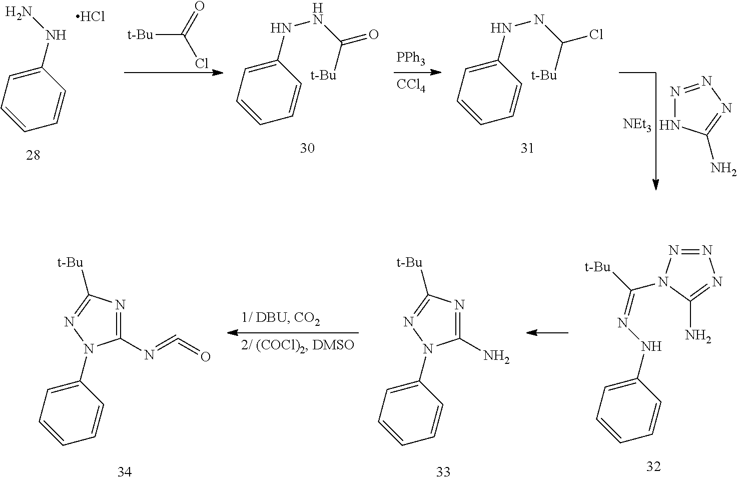

### Step 1. Synthesis of Compound (30)

To a solution of phenylhydrazine hydrochloride 28 (4.0 g, 27.68 mmol) in 100 mL of DCM and Et₃N (8.1 mL, 2.1 eqv) at 5 C was added solution of pivaloyl chloride (3.5 g, 1.05 eqv) in 20 mL DCM dropwise. Mixture stirred at r.t. for 5 min. and evaporated. Residue partitioned between EtOAc and water. Organic layer separated, dried over sodium sulfate and evaporated. Product was used in the next step without purification (Yield: 99.6%, 5.3 g).

### Step 2. Synthesis of Compound (31)

To a solution of compound 30 (5.3 g, 27 mmol) in 100 mL of anhydrous CH₃CN was added CBr4 (17.9 g, 2 eqv) and PPh3 (14.5 g, 2 eqv). Mixture stirred at ambient temperature overnight. Volatiles evaporated, residue taken up in 30 mL Et₂O and additionally 50 mL of hexane were added. After 30 min. solution decantated from oily residue and evaporated to afford crude product 31, which was used in the next step without purification (Yield: 48%, 2.8 g)

### Step 3. Synthesis of Compound (32)

To a suspension of 5-aminotetrazole monohydrate (1.13 g, 11 mmol) in EtOH was added Et₃N (1.1 g, 11 mmol). After dissolution, compound 31 (2.8 g, 11 mmol) was added in one portion. Precipitate start to form after several minutes. Mixture stirred at r.t. for 2 h and filtered, washed with EtOH and precipitate dried on air. Yield: 52%, 1.5 g of white solid.

### Step 4. Synthesis of Compound (33)

Compound 32 (1.5 g, 5.7 mmol) suspended in 20 mL of o-xylene and subjected to reflux for 20 min. Starting material dissolves upon heating and product crystallize from reaction mixture after cooling. Precipitate filtered off, washed with toluene and dried on air. Yield of compound 32: 89%, 1.1 g of white crystals

### Step 5. Synthesis of Compound (34): 3-cyclopropyl-5-isocyanato-1-phenyl-1H-1,2,4-triazole

A solution of oxalyl chloride (200 mL, 2.31 mmol) in 5 mL CH₂Cl₂ under an atmosphere of CO₂ was cooled to −60° C. and DMSO (0.18 g, 2.31 mmol) was added dropwise to the cold solution as a solution in 1.0 mL CH₂Cl₂. The resulting mixture was stirred at low temperature for 5 min. In a second flask, CO₂ was bubbled through a solution of compound 33 (0.5 g, 2.31 mmol) and DBU (0.36 g, 2.31 mmol) in 5 mL CH₂Cl₂ at room temperature. This mixture was cannula transferred to the first (cold) solution. The resulting cold mixture was stirred at low temperature for 10 min. Triethylamine (0.32 mL, 2.31 mmol) was added, and the mixture was stirred for 10 min. The solution was allowed to slowly warm to room temperature and was left to stir for an additional 10 min. The mixture was concentrated under reduced pressure to afford the crude material, which was purified by flash chromatography on dried silica gel eluting EtOAc:Hex=1:2 to afford 130 mg (Yield: 23%) of the isocyanate 34 (Probably in dimeric or tetrameric form, ESI/MS M+1=243.6, 485.6, 727.8, 969.7). ¹H-NMR in CDCl₃ ppm: 1.49 (d, 9H), 7.36-7.56 (m, 4H), 8.05 (d, 1H)

### Synthesis of 38: tert-butyl((4-isocyanatonaphthalen-1-yl)oxy)dimethylsilane

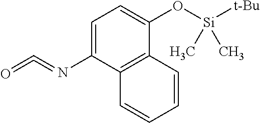 [SMILES: CC(C)(C)[Si](C)(C)Oc1ccc(N=C=O)c2ccccc12]

### Step1. Synthesis of Compound (36)

TBDMS-Cl (2.2 g, 14.6 mmol, 1.2 eq) was added to a solution of 4-nitronaphthalen-1-ol 35 (2.3 g, 12.2 mmol, 1 eq) and imidazole (0.99 g, 14.6 mmol, 1.2 eq) in THF (100 mL) and the reaction mixture was stirred at ambient temperature for 12 h. The formed precipitate was filtered, the solvent was evaporated under reduced pressure, the residue was purified by column chromatography on silica gel (DCM) (Yield: 96%, 3.6 g).

### Step2. Synthesis of Compound (37)

A mixture of compound 36 (3.6 g, 12.1 mmol, 1 eq) and 10% Pd/C (10% by weight, 0.4 g) in MeOH (20 mL) was stirred at room temperature under flow of H₂ overnight. Then Pd/C was filtered, the solvent was evaporated under reduced pressure. The residue was used directly to the next step without additional purification (Yield: 96%, 3.2 g).

### Step3. Synthesis of Compound (38): tert-butyl((4-isocyanatonaphthalen-1-yl)oxy)dimethylsilane

Triphosgene (3.47 g, 11.7 mmol, 1 eq) was added portions to a solution of compound 37 (3.2 g, 11.7 mmol, 1 eq) and Et₃N (4.89 mL, 35.1 mmol, 3 eq) in DCM (50 mL) at −20° C., the reaction mixture was stirred at ambient temperature overnight. Solvent was removed under reduced pressure, THF (50 mL) was added to the residue, the formed precipitate was filtered off, solvent was evaporated under reduced pressure to give compound 36. The residue was used directly to the next step without additional purification. Yield of compound 38 (Yield: 100%, 3.5 g).

### Synthesis of 40: 4-isocyanato-2-(trifluoromethyl)benzonitrile

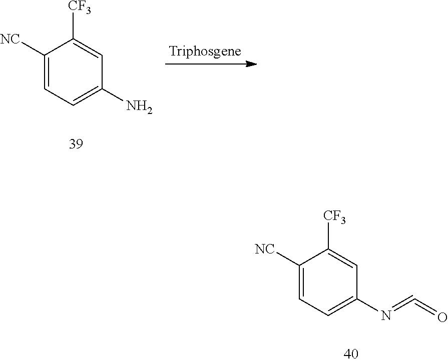 [SMILES: N#Cc1ccc(N)cc1C(F)(F)F ; N#Cc1ccc(N=C=O)cc1C(F)(F)F]

### Synthesis of Compound (40): 4-isocyanato-2-(trifluoromethyl)benzonitrile

To a solution of triphosgene (73 mg, 0.25 mmol) in 1 mL of anhydrous 1,4-dioxane was added solution of 4-amino-2-(trifluoromethyl)benzonitrile 39 (115 mg, 0.62 mmol) in 2 mL of anhydrous 1,4-dioxane at room temperature. After 10 min mixture subjected to reflux for 1 h with gas bubbler. After cooling and evaporation 120 mg of crude isocyanate 40 was used in the next step without purification.

### Synthesis of Compound 42: 5-chloro-2-isocyanato-4-(trifluoromethyl)pyridine

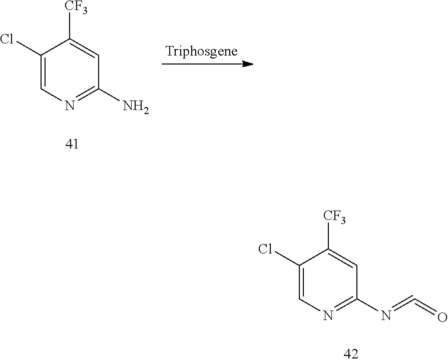 [SMILES: Nc1cc(C(F)(F)F)c(Cl)cn1 ; O=C=Nc1cc(C(F)(F)F)c(Cl)cn1]

### Synthesis of Compound (42): 5-chloro-2-isocyanato-4-(trifluoromethyl)pyridine

To a solution of triphosgene (73 mg, 0.25 mmol) in 1 mL DCM at OC solution of 5-chloro-4-(trifluoromethyl)pyridin-2-amine 41 (120 mg, 0.62 mmol) and DIPEA (168 mg, 1.3 mmol) in 2 mL DCM was added dropwise under stirring. After 10 min mixture evaporated, residue taken up in anhydrous THF and filtered. The filtrate evaporated to afford 120 mg of crude isocyanate 42, which was used in next step without purification.

### Synthesis of 44: 1-chloro-4-isocyanato-2-(trifluoromethyl)benzene

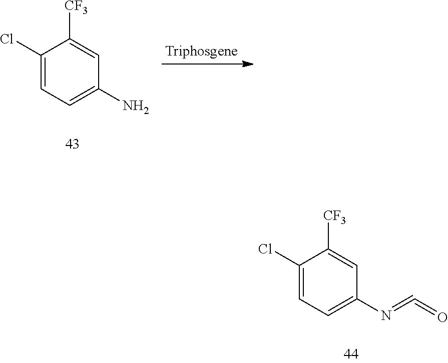 [SMILES: Nc1ccc(Cl)c(C(F)(F)F)c1 ; O=C=Nc1ccc(Cl)c(C(F)(F)F)c1]

### Synthesis of Compound (44): 1-chloro-4-isocyanato-2-(trifluoromethyl)benzene

To a solution of triphosgene (73 mg, 0.25 mmol) in 1 mL of anhydrous 1,4-dioxane was added solution of 4-chloro-3-(trifluoromethyl)aniline 43 (121 mg, 0.62 mmol) in 2 mL of anhydrous 1,4-dioxane at room temperature. After 10 min mixture subjected to reflux for 1 h with gas bubbler. After cooling and evaporation 132 mg of crude isocyanate 44 was used in the next step without purification.

### Synthesis of 50: 8-((5-aminopyridin-2-yl)oxy)pyrido[2,3-b]pyrazin-3(4H)-one

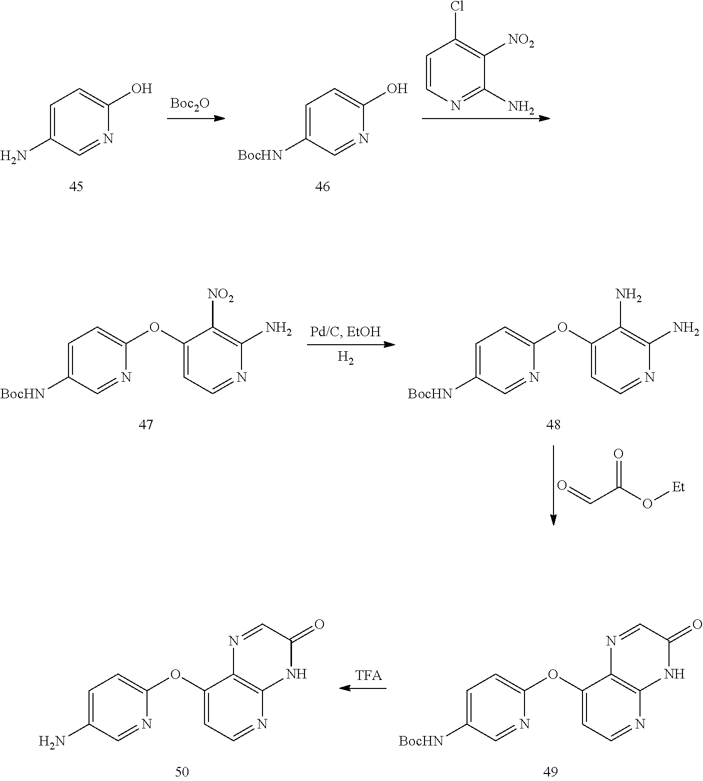

### Step 1. Synthesis of Compound (46)

To solution of compound 45 (2 g, 18.2 mmol) in t-BuOH (40 mL) was added (Boc)₂O (4 g, 18.2 mmol), solution stirred and refluxed 18 h, then cooled, solvent concentrated under reduced pressure, residue crystallization from toluene, dried yield compound 46 (Yield: 37%, 1.5 g).

### Step 2. Synthesis of Compounds (47)

NaH (0.23 g, 5.8 mmol) was added portion wise to a solution of compound 46 (0.98 g, 5.8 mmol) in dry DMSO (30 mL) and stirred at ambient temperature 30 min. 4-Chloro-3-nitropyridin-2-amine (1.0 g, 5.8 mmol) was added as a solid in one portion and the reaction mixture was subsequently heated at 70° C. for 1 h. The reaction mixture was cooled, diluted with water (200 mL) and extracted with EtOAc (2×50 mL) The organic layer was separated, washed with brine, dried over sodium sulfate, filtered and concentrated. The residue after evaporation was subjected to column chromatography on silica gel eluting with dichloromethane/ethyl acetate (0→30%) to afford compound 47 (Yield: 50%, 1.0 g).

### Step 3. Synthesis of Compounds (48)

10% Pd/C (0.5 g, ˜10% weight) were added to a solution of compound 47 (1.0 g, 2.88 mmol) in mixture EtOH/EtOAc (50 mL, 2:1). Hydrogen was passed through the reaction mixture under stirring at room temperature for 16 h. Then the catalyst was filtered off through Celite. The filtrate was evaporated under reduced pressure to afford of compound 48 (Yield: 99%, 0.91 g).

### Step 4. Synthesis of Compounds (49)

To a solution of compound 48 (910 mg, 2.87 mmol) in EtOH (50 mL) was added ethyl glyoxalate 50% solution in toluene (1.5 mL, 7. mmol). Then reaction mixture reflux for 2 h. The solvent was evaporated.

The residue after evaporation was subjected to column chromatography on silica gel eluting with dichloromethane/ethyl acetate (0→100%) to afford the compound 49 (Yield: 11%, 0.11 g).

### Step 5. Synthesis of Compounds (50): 8-((5-aminopyridin-2-yl)oxy)pyrido[2,3-b]pyrazin-3(4H)-one

To solution of compound 49 (110 mg, 0.31 mmol) in TFA (2 mL) was stirred 1 h at ambient temperature, when evaporated under reduced pressure, residue dissolved in water (7 mL), pH of solution up to 6 with NaHCO₃, water evaporated under reduced pressure to dryness. Residue 50 dissolved in EtOH (30 mL), filtered, evaporated under reduced pressure to dryness and used on the next stage without separation.

### Synthesis of 52: 6-aminopyridin-3-yl tert-butyl carbonate

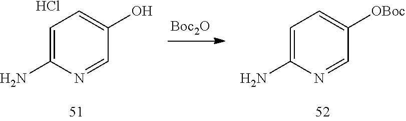

### Synthesis of Compound (52): 6-aminopyridin-3-yl tert-butyl Carbonate

Compound 51 (0.600 g, 4.1 mmol), (Boc)₂O (0.893 g, 4.1 mmol), DMAP (30 mg, 0.02 mmol) and Et₃N (0.414 g, 4.1 mmol) in 30 mL MeCN were stirred at ambient temperature for 12 h and evaporated to dryness. The residue was dissolved in DCM, washed with water and brine, dried under Na₂SO₄ and filtrated. The filtrate was evaporated to dryness afford pure compound 52 (Yield: 79%, 0.679 g).

### Synthesis of 54: Phenyl (3-(tert-butyl)-1-phenyl-1H-pyrazol-5-yl)carbamate

H.-G. Lee, M.-J. Kim, S.-E. Park, J.-J. Kim, S.-G. Lee, Y.-J. Yoon, Synlett, 2009, 2809-2814

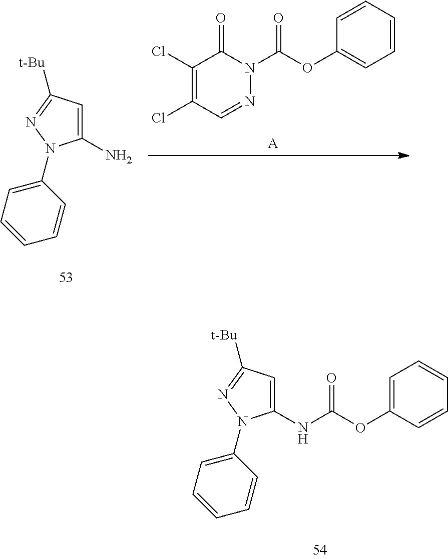

### Synthesis of Compound (54): phenyl (3-(tert-butyl)-1-phenyl-1H-pyrazol-5-yl)carbamate

Mixture of compound 53 (0.226 g, 1.05 mmol) and compound A (0.31 g, 1.1 mmol) in 20 mL of anhydrous THF was refluxed overnight. Volatiles evaporated and product 54 isolated by column chromatography on silica gel eluting DCM:EtOAc (9:1) (Yield: 57%, 0.2 g).

### Synthesis of 56: 3-(tert-butyl)-1-phenyl-1H-pyrazole-5-carbonyl Azide

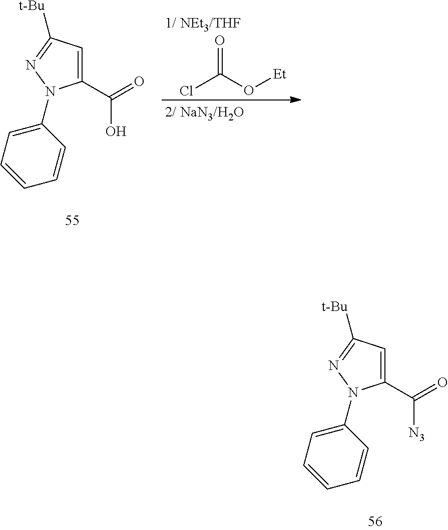 [SMILES: CC(C)(C)c1cc(C(=O)O)n(-c2ccccc2)n1 ; CCOC(=O)Cl ; CC(C)(C)c1cc(C(=O)N=[N+]=[N-])n(-c2ccccc2)n1]

### Synthesis of Compound (56): 3-(tert-butyl)-1-phenyl-1H-pyrazole-5-carbonyl Azide

Under inert atmosphere (Argon), a stirred solution of 3-(tert-butyl)-1-phenyl-1H-pyrazole-5-carboxylic acid 55 (1.98 mmol, 484 mg) and Et₃N (2.57 mmol, 0.35 mL) in dry THF (5 mL) was cooled to 10° C. Ethyl chloroformate (2.97 mmol, 0.280 mL) was added drop wise and the resulting mixture was stirred for 0.5 h. Afterwards, a solution of sodium azide (3.96 mmol, 266 mg) in water (2 mL) was added in one portion. After 1 h at 10° C., the reaction was found to be complete (TLC) and was quenched into iced water (5 mL). The mixture was extracted with EtOAc (3×10 mL) and the combined organic layers were successively dried over MgSO₄, filtered and evaporated to obtain a crude azide 56.

### Synthesis of 59: tert-butyl(4-isocyanato-3-(trifluoromethyl)phenoxy)dimethylsilane

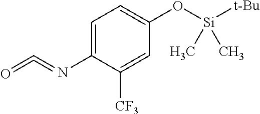 [SMILES: CC(C)(C)[Si](C)(C)Oc1ccc(N=C=O)c(C(F)(F)F)c1]

### Step 1. Synthesis of Compound (58)

TBDMS-Cl (1.022 g, 6.78 mmol) was added to a solution of 4-amino-3-(trifluoromethyl)phenol 57 (1.0 g, 5.65 mmol) and imidazole (0.461 g, 6.78 mmol) in THF (25 mL), the reaction mass was stirred at room temperature for 12 h. The formed precipitate was filtered, the solvent from the mother liquor was removed under reduced pressure, the residue was purified by column chromatography, using hexane and hexane/Et₂O 2:1, 1:1 as, to provide the product 58 (Yield: 91.5%, 1.5 g).

### Step 2. Synthesis of Compound (59): tert-butyl(4-isocyanato-3-(trifluoromethyl)phenoxy)dimethylsilane

Under Ar triphosgene (1.531 g, 5.15 mmol) was added portionwise to a solution of 58 (1.5 g, 5.15 mmol) and Et₃N (1.4 mL, 10.3 mmol) in CH₂Cl₂ (25 mL) at −20° C., the reaction mixture was stirred at room temperature for 1 h. Then the solvent was removed under reduced pressure and THF (25 mL) was added to the residue. The formed precipitate was filtered, the solvent from the mother liquor was removed under reduced pressure to get 59.

### Synthesis of 61: Tert-Butyl (3-hydroxyphenyl)carbamate

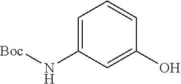 [SMILES: CC(C)(C)OC(=O)Nc1cccc(O)c1]

### Synthesis of Compounds (61)

(Boc)₂O (10.0 g, 45.9 mmol) was added by portions to a solution of 3-aminophenol 60 (5.0 g, 45.9 mmol) and Et₃N (6.5 mL, 45.9 mmol) in DCM (200 mL). The reaction mixture was stirred at ambient temperature for 2 h and evaporated to dryness. The residue was purified by flash chromatography on silica gel with DCM to afford compound 61 (Yield: 64%, 6.0 g).

### Synthesis of 64: 4-(3-aminophenoxy)-N-methylpicolinamide

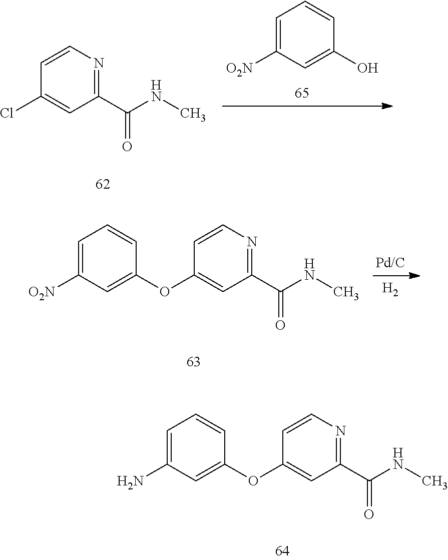 [SMILES: CNC(=O)c1cc(Cl)ccn1 ; O=[N+]([O-])c1cccc(O)c1 ; CNC(=O)c1cc(Oc2cccc([N+](=O)[O-])c2)ccn1 ; "H2" ; CNC(=O)c1cc(Oc2cccc(N)c2)ccn1]

### Step 1. Synthesis of Compound (63)

4-Chloro-2-methylcarboxamidepyridine 62 (0.5 g, 2.93 mmol, 1.0 eq) was added to 3-nitrophenol 65 (0.45 g, 3.2 mmol, 1.1 eq) and the mixture was heating overnight at 150° C., after cooling brown precipitate was formed (Yield: 98%, 0.8 g).

### Step 2. Synthesis of Compound (64): 4-(3-aminophenoxy)-N-methylpicolinamide

0.8 g of compound 63 in 100 mL of MeOH was hydrogenated on 80 mg 10% palladium on carbone at 10 bars for 4 h at room temperature. The catalyst was filtered off, solvent was removed under reduced pressure to give compound 64. The residue was used directly to the next step without additional purification (Yield: 91%, 0.7 g).

### Synthesis of 66: Phenyl (3-(tert-butyl)-1-phenyl-1H-1,2,4-triazol-5-yl)carbamate

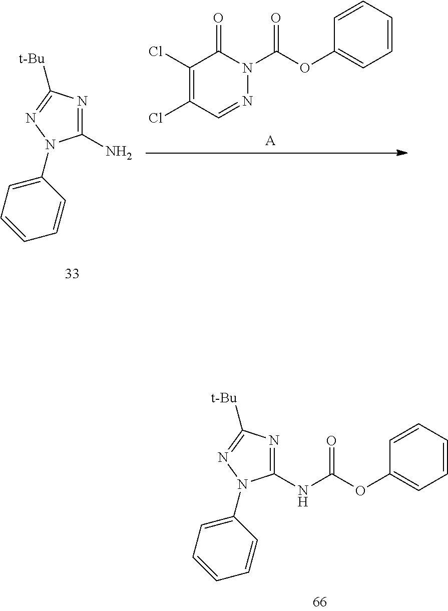

Synthesis of Compound (66): phenyl (3-(tert-butyl)-1-phenyl-1H-1,2,4-triazol-5-yl)carbamate Mixture of 3-(tert-butyl)-1-phenyl-1H-1,2,4-triazol-5-amine 33 (0.5 g, 2.32 mmol) and compound A (0.71 g, 2.5 mmol) in 20 mL of anhydrous THF was refluxed overnight. Volatiles was evaporated and product 66 was isolated by column chromatography on silica gel eluting DCM:EtOAc (9:1) (Yield: 51%, 400 mg).

### Synthesis of 69: 5-(3-aminophenoxy)-N-methylpicolinamide

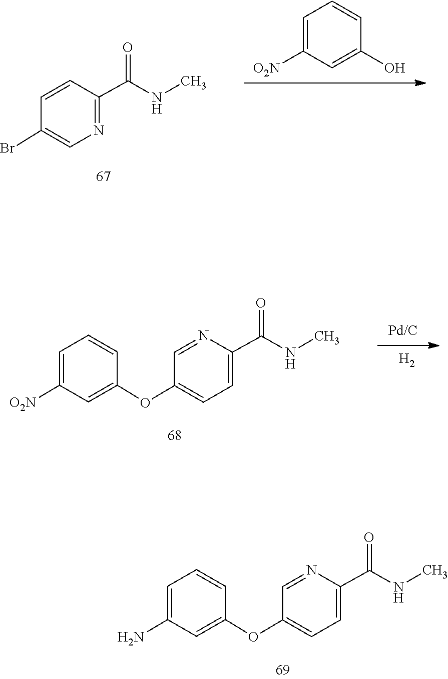 [SMILES: CNC(=O)c1ccc(Br)cn1 ; O=[N+]([O-])c1cccc(O)c1 ; CNC(=O)c1ccc(Oc2cccc([N+](=O)[O-])c2)cn1 ; "H2" ; CNC(=O)c1ccc(Oc2cccc(N)c2)cn1]

### Step 1. Synthesis of Compound (68)

Mixture of 5-Bromo-2-methylcarboxamidepyridine 67 (1.0 g, 4.65 mmol, 1.0 eq), 3-nitrophenol (1.29 g, 9.3 mmol, 2.0 eq) and K₂CO₃ (0.64 g, 4.65 mmol) in 30 mL of NMP was heating overnight at 150° C., after cooling water was added and product was extracted with CHCl₃. Organic extract was dried over sodium sulfate and evaporated off. Residue was triturated with acetone, brown precipitate was filtered and dried (Yield: 16%, 0.2 g).

### Step 2. Synthesis of Compound (69)

0.2 g of compound 68 in 100 mL of MeOH was hydrogenated on 20 mg 10% palladium on carbone at 10 bars for 4 h at room temperature. The catalyst was filtered off, solvent was removed under reduced pressure to give compound 69. The residue was used directly to the next step without additional purification (Yield: 91%, 0.19 g).

### Synthesis of 71: 2-fluoro-4-phenoxyaniline

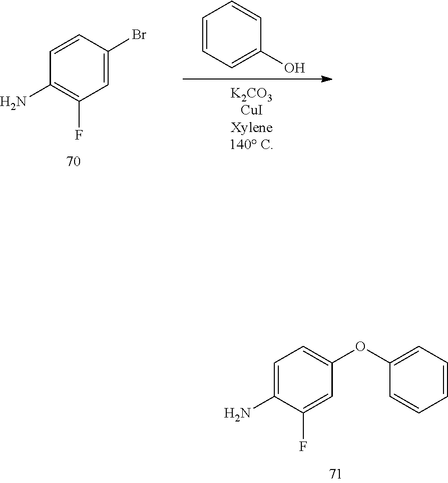 [SMILES: Nc1ccc(Br)cc1F ; Oc1ccccc1 ; Nc1ccc(Oc2ccccc2)cc1F]

### Synthesis of Compound (71): 2-fluoro-4-phenoxyaniline

A mixture of aniline 70 (1.0 g, 5.26 mmol), phenol (1.0 g, 10.52 mmol), N-methyl-imidazole (0.216 g, 2.63 mmol), K₂CO₃ (1.452 g, 10.52 mmol) and CuI (0.05 g, 0262 mmol) in xylene (20 mL) was stirred in glass “bomb” at 140° C. for 30 h. After cooling to room temperature the reaction mass was filtered, the mother liquor was concentrated under reduced pressure. The residue was purified by column chromatography, using hexane and hexane/Et₂O 5:1 as eluent, to provide the product 71 (Yield: 9.2%, 0.098 g).

### Synthesis of 72: 2-fluoro-4-(pyridin-4-yloxy)aniline

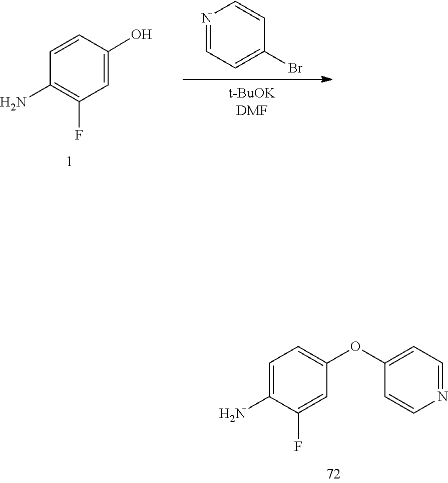 [SMILES: Nc1ccc(O)cc1F ; Brc1ccncc1 ; Nc1ccc(Oc2ccncc2)cc1F]

### Synthesis of Compound (72): 2-fluoro-4-phenoxyaniline

A mixture of 1 (3.41 g, 26.8 mmol), 4-bromo-pyridine hydrochloride (5.212 g, 26.8 mmol) and t-BuOK (6.6 g, 59.0 mmol) in DMF (200 mL) was stirred at 50° C. for 12 h. After cooling to room temperature the reaction mass was filtered, the mother liquor was concentrated under reduced pressure. The residue was purified by column chromatography, using CH₂Cl₂ and 2% MeOH in CH₂Cl₂ as eluent, to provide the product 72 (Yield: 3.1%, 0.17 g).

### Synthesis of 75: N-(4-(4-amino-3-fluorophenoxy)pyridin-2-yl)acetamide

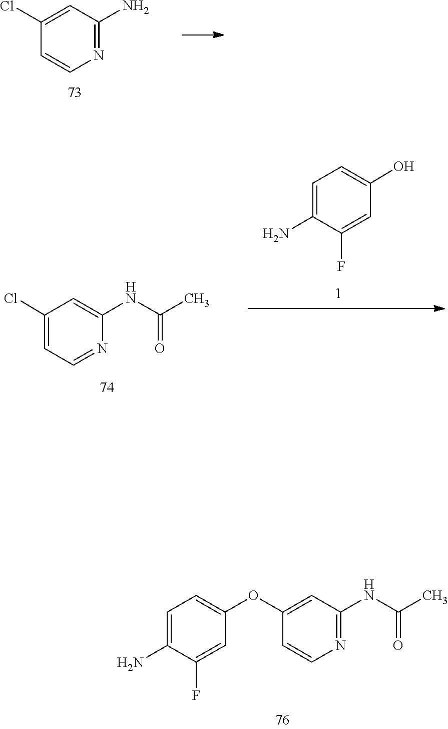 [SMILES: Nc1cc(Cl)ccn1 ; Nc1ccc(O)cc1F ; CC(=O)Nc1cc(Cl)ccn1 ; CC(=O)Nc1cc(Oc2ccc(N)c(F)c2)ccn1]

### Step 1. Synthesis of Compound (74)

To a compound 73 (1.74 g, 13.5 mmol) was added acetic anhydride (1.66 g, 16 mmol) and triethylamine (2.74 g, 27 mmol). The mixture was stirred overnight at 60° C. The reaction mixture was concentrated under reduced pressure. The crude residue was purified by silica gel chromatography (Yield: 67%, 1.55 g).

### Step 2. Synthesis of Compound (75)

Compound 74 (0.62 g, 3.65 mmol), compound 2 (0.58 g, 4.6 mmol) and Cs₂CO₃ (1.54 g, 4.72 mmol) was mixed in NMP (15 mL). After stirring for 12 h at 100° C., reaction mixture cooling to room temperature, water (100 mL) was added to the reaction mixture, and the resulting mixture was extracted with EtOAc (2×50 mL). The combined organic extracts were washed with brine and dried over Na₂SO₄. The solvent was then removed under reduced pressure. The crude product was purified with column using PE/EA as eluent to give compound 75 (Yield: 9%, 0.11 g).

### Synthesis of 76: 4-(4-amino-3-fluorophenoxy)-N-methylpicolinamide

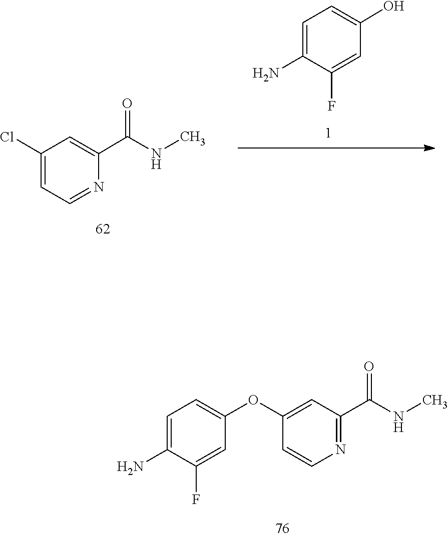 [SMILES: CNC(=O)c1cc(Cl)ccn1 ; Nc1ccc(O)cc1F ; CNC(=O)c1cc(Oc2ccc(N)c(F)c2)ccn1]

### Synthesis of Compound (76): 4-(4-amino-3-fluorophenoxy)-N-methylpicolinamide

Compound 62 (1.34 g, 7.87 mmol), compound 1 (1 g, 7.8 mmol) and Cs₂CO₃ (1.54 g, 4.72 mmol) was mixed in NMP (15 mL). After stirring for 12 h at 100° C., reaction mixture cooling to room temperature, water (100 mL) was added to the reaction mixture, and the resulting mixture was extracted with EtOAc (2×50 mL). The combined organic extracts were washed with brine and dried over Na₂SO₄. The solvent was then removed under reduced pressure. The crude product was purified with column using PE/EA as eluent to give compound 76 (Yield: 29%, 0.6 g).

### Synthesis of 80: 8-(4-amino-3-fluorophenoxy)-2-methylpyrido[2,3-b]pyrazin-3(4H)-one

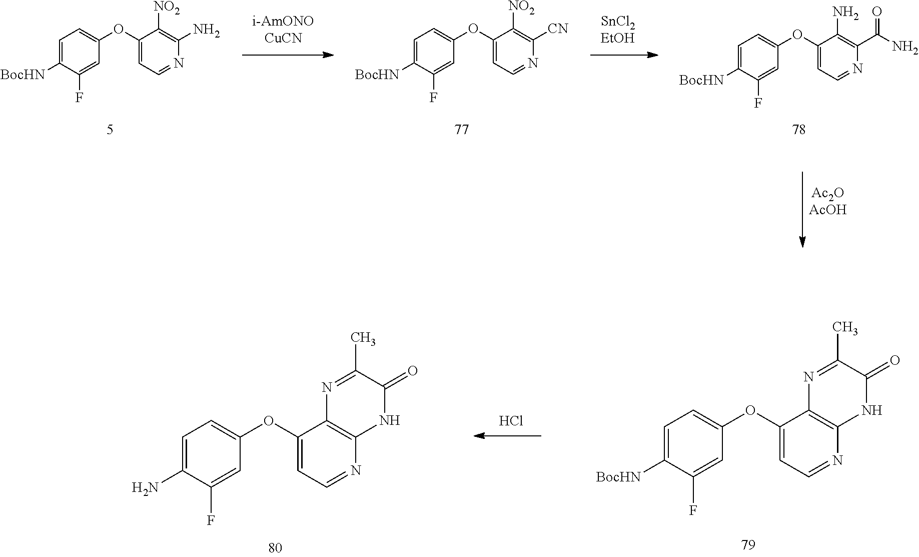 [SMILES: CC(C)(C)OC(=O)Nc1ccc(Oc2ccnc(N)c2[N+](=O)[O-])cc1F ; CC(C)(C)OC(=O)Nc1ccc(Oc2ccnc(C#N)c2[N+](=O)[O-])cc1F ; CC(C)(C)OC(=O)Nc1ccc(Oc2ccnc(C(N)=O)c2N)cc1F ; Cc1nc2c(Oc3ccc(N)c(F)c3)ccnc2[nH]c1=O ; Cl ; Cc1nc2c(Oc3ccc(NC(=O)OC(C)(C)C)c(F)c3)ccnc2[nH]c1=O]

### Step 1. Synthesis of Compound (77)

Mixture of compound 5 (5.6 g, 15.38 mmol) and CuCN (2.07 g, 23.07 mmol) in 100 mL of anhydrous CH₃CN was subjected to reflux and i-AmONO (2.34 g, 20.0 mmol) was added dropwise. Reflux continued for 2 h and mixture evaporated to dryness. Residue taken up in EtOAc-THF (9:1) and filtered through celite. Filtrate evaporated and product purified by column chromatography on silica gel using EtOAc as eluent (Yield: 26%, 1.5 g).

### Step 2. Synthesis of Compound (78)

Compound 77 (1.5 g, 4.0 mmol) dissolved in 30 mL of EtOH and SnCl₂*2H₂O (4.51 g, 5 equivalents) was added in one portion. Mixture stirred at ambient temperature for 2 h. TLC indicated complete conversion. Volatiles evaporated, residue taken up EtOAc and quenched with 100 mL of a saturated solution of NaHCO₃. Mixture filtered through celite and organic layer was separated, washed with brine, dried over sodium sulfate, filtered and concentrated to afford compound 78 (1.45 g), which was pure enough to be used in the next step without purification.

### Step 3. Synthesis of Compound (79)

Ac₂O (2.0 g, 19.6 mmol) were added to a solution of compound 78 (1.45 g, 4.0 mmol) in glacial AcOH (20 mL) at room temperature. Mixture stirred at room temperature for 2 h and heated at 50° C. for 30 min. Volatiles evaporated residue taken up EtOAc and quenched with 30 mL of a saturated solution of NaHCO₃. Precipitated product filtered, washed with water, EtOAc and dried in air to afford 0.3 g of compound 79. Additional amount of product was left in mother liquor.

### Step 4. Synthesis of Compound (80): 8-(4-amino-3-fluorophenoxy)-2-methylpyrido[2,3-b]pyrazin-3(4H)-one

HCl/dioxane (2.1 mL, 6.3 mmol, 3M) was added to compound 79 (300 mg, 0.77 mmol). The reaction mixture stirred at ambient temperature overnight with gas-stop bubbler. Mixture evaporated under reduced pressure, then Et₃N (1 mL) in 5 mol MeOH were added. Mixture stirred for 5 min and evaporated. Then 1,4-dioxane (5 mL) was added and precipitate was filtered off. Filtrate evaporated to dryness to afford the compound 80 (Yield: 91%, 200 mg) as a white solid.

### Synthesis of 81: 2-fluoro-4-(4-fluorophenoxy)aniline

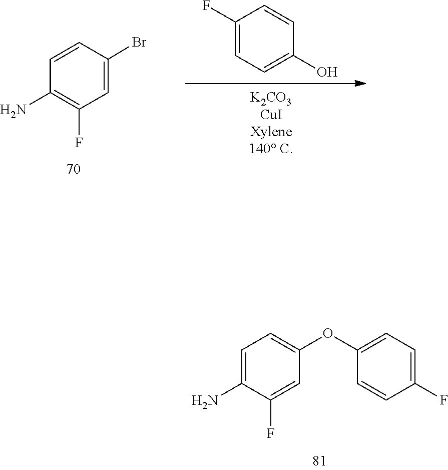 [SMILES: Nc1ccc(Br)cc1F ; Oc1ccc(F)cc1 ; Nc1ccc(Oc2ccc(F)cc2)cc1F]

### Synthesis of Compound (81): 2-fluoro-4-(4-fluorophenoxy)aniline

A mixture of aniline 70 (1.0 g, 5.26 mmol), 4-fluoro-phenol (0.707 g, 6.31 mmol), N-methyl-imidazole (0.216 g, 2.63 mmol), K₂CO₃ (1.452 g, 10.52 mmol) and CuI (0.05 g, 0.262 mmol) in xylene (20 mL) was stirred in glass “bomb” at 140° C. for 30 h. After cooling to room temperature, the reaction mass was filtered, the mother liquor was concentrated under reduced pressure. The residue was purified by column chromatography, using hexane and hexane/Et₂O 5:1 as eluent, to provide the product 81 (Yield: 9.5%, 0.11 g).

### Synthesis of 89: 2-phenyl-5,6-dihydro-4H-pyrrolo[1,2-b]pyrazole-3-carboxylic Acid

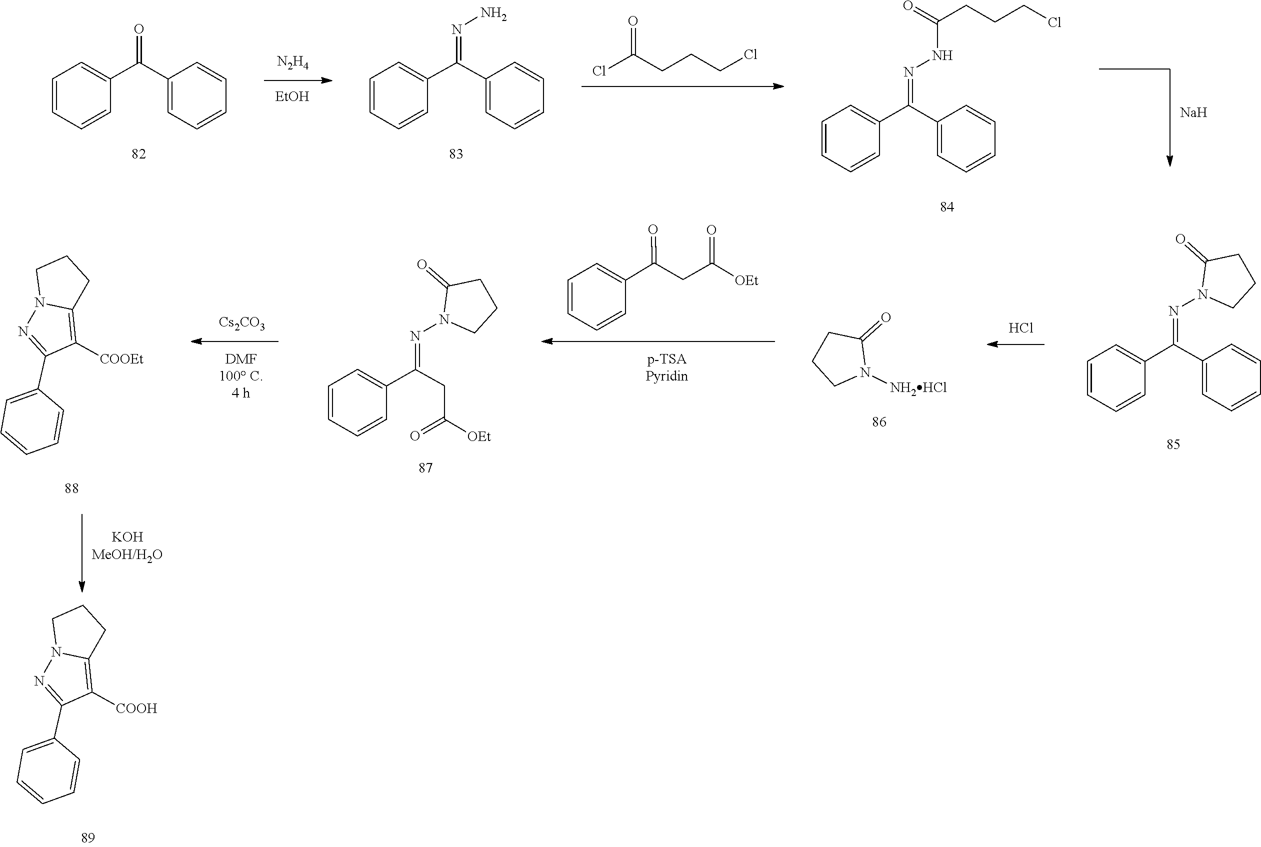

### Step 1. Synthesis of Compound (83)

A mixture of 82 (31.0 g, 170.3 mmol) and N₂H₄*H₂O (11.29 g, 238.4 mmol) in EtOH (100 mL) was stirred at boiling temperature for 36 h. The solvent was removed under reduced pressure, the residue was dissolved in CH₂Cl₂ (200 mL), washed with water (50 mL), from aqua layer the product was extracted with CH₂Cl₂ (2×30 mL), the combined organic layers were dried under Na₂SO₄, the solvent was removed under reduced pressure to provide the product 83 (Yield: 91%, 30.4 g).

### Step 2. Synthesis of Compound (84)

4-Chlorobutyryl chloride (21.9 g, 155 mmol) was added dropwise at 0° C. to a solution of 83 (30.4 g, 155 mmol) and pyridine (12 g, 155 mmol) in CH₂Cl₂ (250 mL), the reaction mass was stirred at room temperature for 12 h. Then the mixture was washed with saturated solution of K₂CO₃ (2×100 mL), the solvent was removed under reduced pressure, the residue was purified by crystallization from hexane to provide the product 84 (Yield: 79%, 36.9 g).

### Step 3. Synthesis of Compound (85)

NaH (4.91 g, 122.7 mmol) was added in portions to a solution of 84 (36.9 g, 122.7 mmol) in THF (300 mL), the reaction mass was stirred at room temperature for 12 h. Then saturated solution of NH₄Cl (50 mL) was added, the mixture was stirred for 30 min., water was added, the product was extracted with EtOAc (2×200 mL), the organic layers were dried under Na₂SO₄, the solvents were removed under reduced pressure. The residue was purified by crystallization from hexane/Et₂O 3:1 to provide the product 85 (Yield: 82.4%, 26.7 g).

### Step 4. Synthesis of Compound (86)

A mixture of 85 (24.3 g, 92 mmol) and HCl (aqua 37%, 75 mL) in THF (150 mL) was stirred at room temperature for 12 h. The mixture was washed with EtOAc (3×50 mL), the residue was reevaporated under reduced pressure with EtOH/toluene 1:1 (3×150 mL) and purified by crystallization from CH₃CN to provide the product 86 (Yield: 55.7%, 7.0 g).

### Step 5. Synthesis of Compound (87)

A mixture of 86 (6.5 g, 47.6 mmol), Ethyl benzoylacetate (9.143 g, 47.8 mmol) and p-TSA (0.05 g) in pyridine (100 mL) was stirred at boiling temperature for 12 h. The solvent was removed under reduced pressure, the residue was reevaporated under reduced pressure with toluene (3×50 mL) and then purified by column chromatography, using CH₂Cl₂ and 2% MeOH in CH₂Cl₂ as eluent, to provide the product 87 (Yield: 31.4%, 4.1 g).

### Step 6. Synthesis of Compound (88)

A mixture of 87 (4.1 g, 14.9 mmol) and 052003 (9.7 g, 29.9 mmol) in DMF (100 mL) was stirred at room temperature for 30 min, at 100° C. for 4 h. Then hexane (50 mL), Et₂O (50 mL) and EtOAc (50 mL) were added, the precipitate was filtered, the solvents from the mother liquor were removed under reduced pressure, the residue was purified by column chromatography, using hexane/Et₂O 1:1 as eluent, to provide the product 88 (Yield: 22.1%, 0.847 g).

### Step 7. Synthesis of Compound (89): 2-phenyl-5,6-dihydro-4H-pyrrolo[1,2-b]pyrazole-3-carboxylic Acid

A solution of KOH (1 g) in water (5 mL) was added to a solution of 88 (0.847 g, 3.31 mmol) in MeOH (20 mL), the reaction mass was stirred at room temperature for 12 h. The solvent was removed under reduced pressure, water (20 mL) was added, the solution was acidified with 10% aqua HCl to pH 6, the product was extracted with CH₂Cl₂ (2×30 mL), the combined organic layers were dried under Na₂SO₄, the solvent was removed under reduced pressure to provide the product 89 (Yield: 66.7%, 0.503 g).

### Synthesis of 94: 8-(4-aminophenoxy)pyrido[2,3-b]pyrazin-3(4H)-one

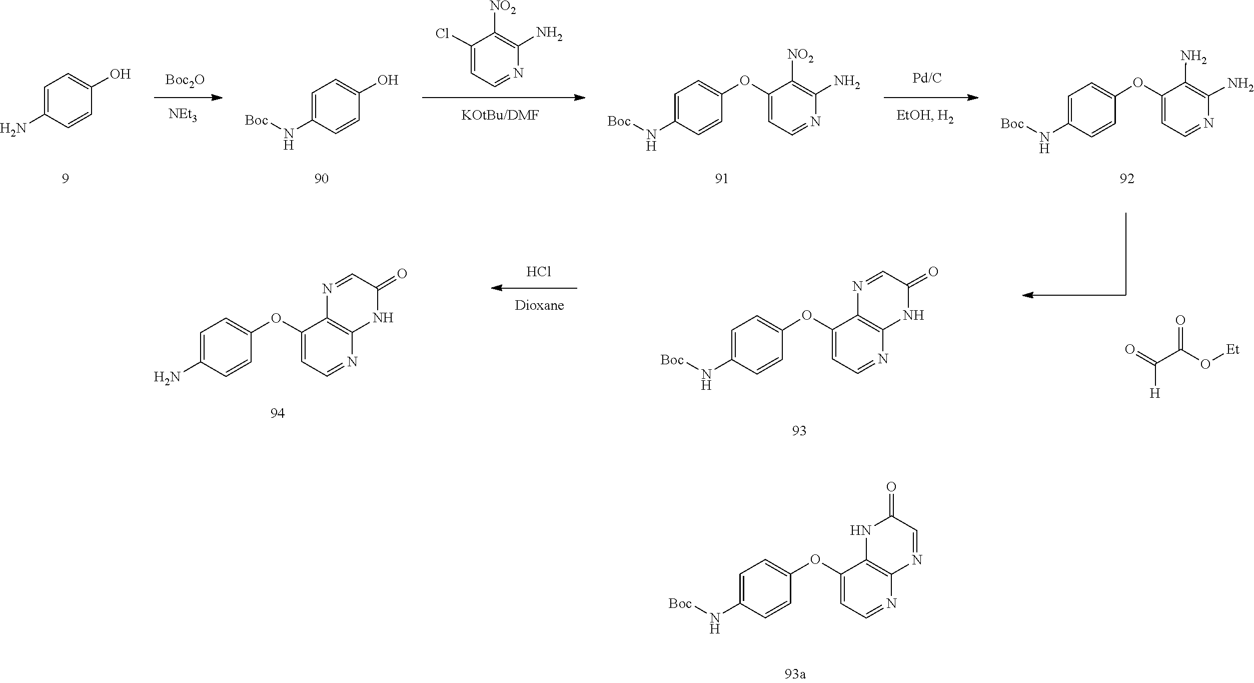

### Step 1. Synthesis of Compound (90)

(Boc)₂O (29.9 g, 0.14 mol) was added by portions to a solution of compound 9 (15.0 g, 0.14 mol) and Et₃N (22.0 mL, 0.15 mol) in THF (500 mL). The reaction mixture was stirred at ambient temperature for 2 h and evaporated to dryness. The residue was purified by flash chromatography on silica gel with DCM to afford compound 90 (Yield: 59%, 17 g).

### Step 2. Synthesis of Compound (91)

Potassium tert-butoxide (2.4 g, 0.021 mol) was added portion wise to a solution of compound 90 (3.7 g, 0.017 mol) in dry DMF (150 mL) and stirred at ambient temperature 30 min. 4-Chloro-3-nitropyridin-2-amine (3.04 g, 0.017 mmol) was added as a solid in one portion and the reaction mixture was subsequently heated at 85° C. for 4 hours. The reaction mixture was cooled, diluted with water (100 mL) and formed precipitate was filtered off, washed with water, hexane and dried to afford compound 91 (Yield: 81%, 5.0 g).

### Step 3. Synthesis of Compound (92)

10% Pd/C (0.5 g, ˜10% weight) were added to a solution of compound 91 (5.0 g, 0.014 mol) in mixture EtOH/EtOAc (200 mL/2:1). Hydrogen was passed through the reaction mixture under stirring at room temperature for 16 h. Then the catalyst was filtered off through Celite. The filtrate was evaporated under reduced pressure to afford of compound 92 (Yield: 73%, 3.3 g).

### Step 4. Synthesis of Compound (93 and 93a)

To a solution of compound 92 (3.3 g, 0.011 mol) in EtOH (150 mL) was added ethyl glyoxalate 50% solution in toluene (3.2 mL, 0.016 mol). Then reaction mixture stirred at ambient temperature for 16 h. The solvent was evaporated. The residue after evaporation was subjected to column chromatography on silica gel eluting with dichloromethane/ethyl acetate (0→100%) to afford the compound 93 (Yield: 21%, 0.77 g) and compound 93a (Yield: 56%, 2.1 g).

### Step 5. Synthesis of Compound (94): 8-(4-aminophenoxy)pyrido[2,3-b]pyrazin-3(4H)-one

HCl/dioxane (3.7 mL, 0.01 mol, 3M sol) was added to compound 93 (0.77 g, 0.002 mol). The reaction mixture stirred at ambient temperature 2 h, then evaporated under reduced pressure, then dichloromethane (100 mL) and saturated aqueous solution of potassium dicarbonate (10 mL) was added. The organic layer was separated, washed with brine, dried over sodium sulfate, filtered and concentrated to afford the compound 94 (Yield: 86%, 0.48 g) as a brown solid.

### Synthesis of 98: 2,2,2-trichloroethyl (3-(tert-butyl)-1-(quinolin-6-yl)-1H-pyrazol-5-yl)carbamate

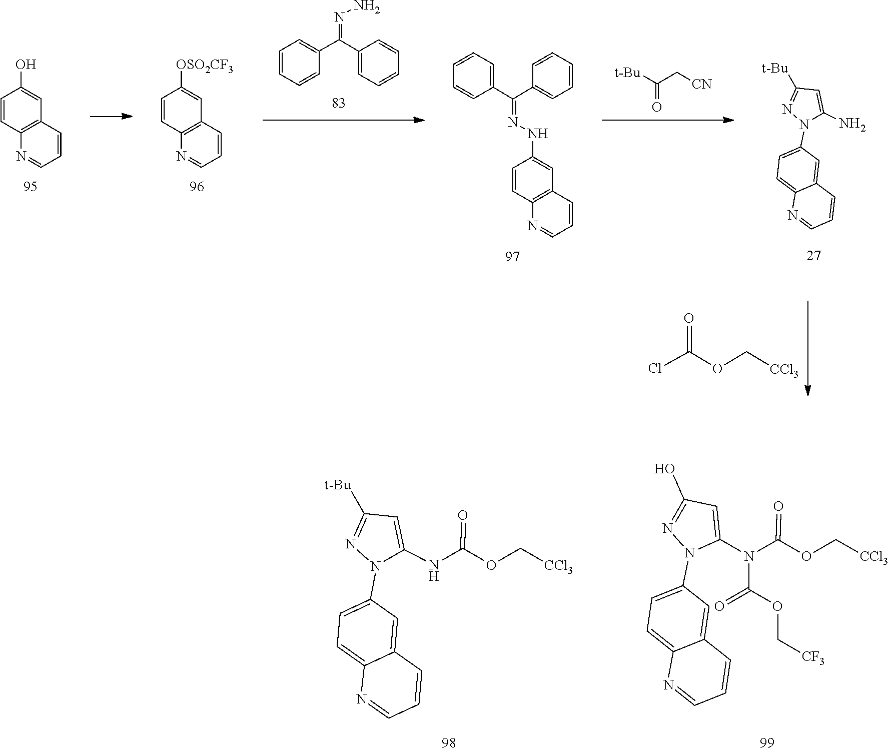

### Step 1. Synthesis of Compound (96)

To a solution of 6-hydroxy-quinoline 95 (1.0 g, 6.89 mmol, 1.0 eq) and pyridine (1.11 mL, 13.78 mmol, 2.0 eq) in 25 mL of dichloromethane was added dropwise at 0° C. a solution of triflic anhydride (1.26 mL, 7.51 mmol, 1.09 eq) in 5 mL of dichloromethane. The mixture was stirred at room temperature for 3 h, poured into water and extracted with dichloromethane. The organic layers were dried over magnesium sulfate, filtered and evaporated under vacuo to afford 6-quinolyl trifluoromethanesulfonate 96 as brown oil (2.0 g, quantitative yield).

### Step 2. Synthesis of Compound (97)

In a sealed tube, crude 6-quinolyl trifluoromethanesulfonate 96 (2.0 g, 6.9 mmol), benzophenone (1.76 g, 8.97 mmol, 1.3 eq), cesium carbonate (3.6 g, 11.04 mmol, 1.6 eq) and 1,1′-ferrocenediyl-bis(diphenylphosphine) (115 mg, 0.21 mmol, 0.03 eq) were put in suspension in 50 mL of toluene. The mixture was degassed with argon for 20 min before addition of palladium acetate (0.6 mg, 0.003 mmol, 0.0004 eq). The mixture was heated at 90° C. for 20 h. After cooling down, the mixture was evaporated, adsorbed on silica gel and purified by flash chromatography using cyclohexane and ethyl acetate to afford N-(benzhydrylideneamino)quinolin-6-amine 97 (Yield: 73%, 1.6 g)

### Step 3. Synthesis of Compound (27)

To a solution of N-(benzhydrylideneamino)quinolin-6-amine 97 (1.6 g, 5.07 mmol, 1.0 eq) and pivaloyl acetonitrile (952 mg, 7.60 mmol, 1.5 eq) in 25 mL of ethanol wad added hydrochloric acid 12N (4.2 mL, 51 mmol, 10 eq). The mixture was stirred at reflux for 20 h. Ethanol was evaporated and the residue was triturated in diethyl ether, diluted in water and washed with ethyl acetate. The aqueous layer was basified with a saturated solution of sodium carbonate (pH 8) and extracted with ethyl acetate. The organic layers were dried over magnesium sulfate, filtered and evaporated under vacuo. The residue was purified by flash chromatography on silica gel using cyclohexane and ethyl acetate to afford 5-tert-butyl-2-(6-quinolyl)pyrazol-3-amine 27 as yellow foam (Yield: 74%, 1.0 g).

### Step 4. Synthesis of Compound (98): 2,2,2-trichloroethyl (3-(tert-butyl)-1-(quinolin-6-yl)-1H-pyrazol-5-yl)carbamate

At −10° C., to a solution of 5-tert-butyl-2-(6-quinolyl)pyrazol-3-amine 27 (1.0 g, 3.75 mmol, 1.0 eq) in 15 mL of dichloromethane were added pyridine (1.0 mL, 12.38 mmol, 3.3 eq), dimethyl aminopyridine (catalytic) and trichloroethyl chloroformate dropwise (0.72 mL, 5.25 mmol, 1.1 eq). The mixture was stirred at −10° C. for 1 h. Water was added. After stirring 10 min at room temperature, the mixture was extracted with dichloromethane. The organic layers were dried over magnesium sulfate, filtered and evaporated under vacuo. The residue was purified by flash chromatography on silica gel using cyclohexane and ethyl acetate to afford 2,2,2-trichloroethyl N-[5-tert-butyl-2-(6-quinolyl)pyrazol-3-yl]carbamate 98 as pale yellow oil (Yield: 43%, 720 mg) and 2,2,2-trichloroethyl N-[5-tert-butyl-2-(6-quinolyl)pyrazol-3-yl]-N-(2,2,2-trichloroethoxycarbonyl)carbamate 99 (1.0 g) as by-product.

### Synthesis of 103: 4-(4-amino-3-(methylthio)phenoxy)-N-methylpicolinamide

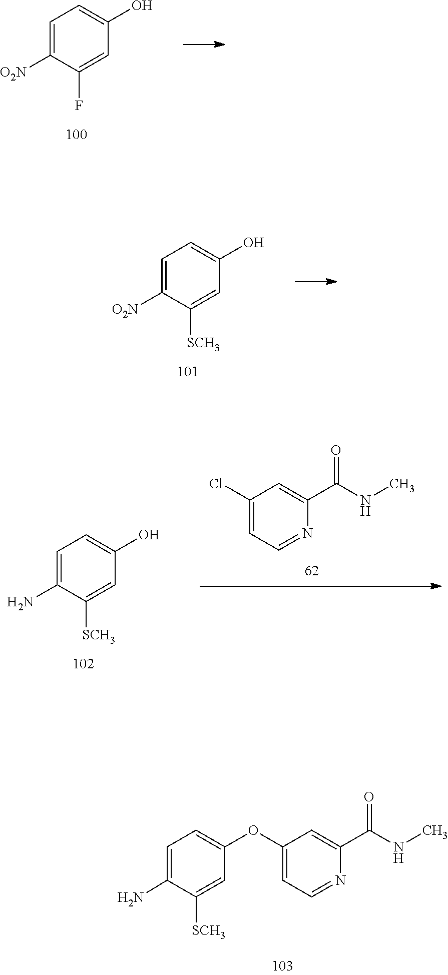 [SMILES: O=[N+]([O-])c1ccc(O)cc1F ; CSc1cc(O)ccc1[N+](=O)[O-] ; CSc1cc(O)ccc1N ; CNC(=O)c1cc(Cl)ccn1 ; CNC(=O)c1cc(Oc2ccc(N)c(SC)c2)ccn1]

### Step 1. Synthesis of Compound (101)

To a solution of 3-fluoro-4-nitrophenol 100 (5.6 g, 35.7 mmol, 1 eq) in 200 mL of dry DMF were added portionwise sodium thiomethoxide (5 g, 71.3 mmol, 2 eq) and potassium carbonate (14.8 g, 107 mmol, 3 eq). The mixture was stirred at room temperature for 48 hours. The suspension was filtrated on a short pad of celite and rinsed with ethyl acetate. The filtrate was concentrated. Water was added, the aqueous phase was acidified to pH 4 with a 1 N solution of hydrochloric acid and extracted with ethyl acetate. The organic layers were dried over magnesium sulfate, filtered and evaporated under vacuo to afford 3-methylsulfanyl-4-nitro-phenol 101 (Yield: 85%, 5.6 g, 85%) as yellow solid.

### Step 2. Synthesis of Compound (102)

Iron powder (11.9 g, 213.8 mmol, 3 eq) was slowly added to a solution of 3-methylsulfanyl-4-nitro-phenol 101 (13.2 g, 71.3 mmol, 1 eq) in 300 mL of acetic acid and 30 mL of ethanol. The reaction mixture was heated up to 45° C. for 18 h. After cooling down, the suspension was filtrated on a short pad of celite and rinsed with methanol. The filtrate was concentrated and adsorbed on silica gel to be purified by flash chromatography using cyclohexane and ethyl acetate. 4-amino-3-methylsulfanyl-phenol 102 (Yield: 55%, 6.1 g) was obtained as brown solid.

### Step 3. Synthesis of Compound (103): 4-(4-amino-3-(methylthio)phenoxy)-N-methylpicolinamide

4-amino-3-methylsulfanyl-phenol 102 (500 mg, 3.22 mmol, 1 eq) was solubilized in 7 mL of DMA. The mixture was degassed with argon before addition of potassium tert-butylate (434 mg, 3.86 mmol, 1.2 eq). After stirring 15 min at room temperature, N-methyl-4-chloro-picolinamide 62 (550 mg, 3.22 mmol, 1 eq) was added and the mixture was heated at 100° C. for 20 h. After cooling down, ethyl acetate and water were added. The aqueous layer was extracted with ethyl acetate and the organic phases were washed with brine. The organic layers were dried over magnesium sulfate, filtered and evaporated under vacuo. The residue was purified by flash chromatography on silica gel using cyclohexane and ethyl acetate to afford 4-(4-amino-3-methylsulfanyl-phenoxy)-N-methyl-pyridine-2-carboxamide 103 (Yield: 41%, 380 mg) as pale orange powder.

### Synthesis of 106: 2,2,2-trichloroethyl (5-(tert-butyl)-1,3,4-thiadiazol-2-yl)carbamate

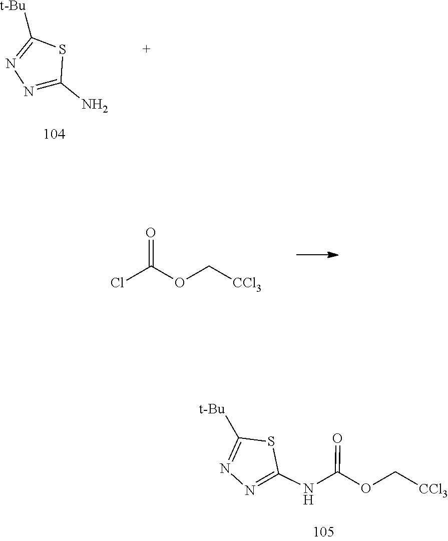

### Synthesis of Compound (105): 2,2,2-trichloroethyl (5-(tert-butyl)-1,3,4-thiadiazol-2-yl)carbamate

At 0° C., pyridine (0.56 mL, 7 mmol, 1.1 eq) and 2,2,2-trichloroethylchloroformate (0.96 mL, 7 mmol, 1.1 eq) were added dropwise to a solution of 5-tert-butyl-1,3,4-thiadiazol-2-amine 104 (1.0 g, 6.36 mmol, 1.0 eq) in 20 mL of THF. The reaction mixture was stirred at 0° C. for 1 h and then at room temperature for 5 h. Water was added and the mixture was extracted with ethyl acetate. The organic layers were washed with water and brine, dried over magnesium sulfate, filtered and evaporated under vacuo to afford 2,2,2-trichloroethyl N-(5-tert-butyl-1,3,4-thiadiazol-2-yl)carbamate 105 (2.26 g, quantitative yield) as white solid.

### Synthesis of 111: 8-(4-amino-3-(methylthio)phenoxy)pyrido[2,3-b]pyrazin-3(4H)-one, Hydrochloride

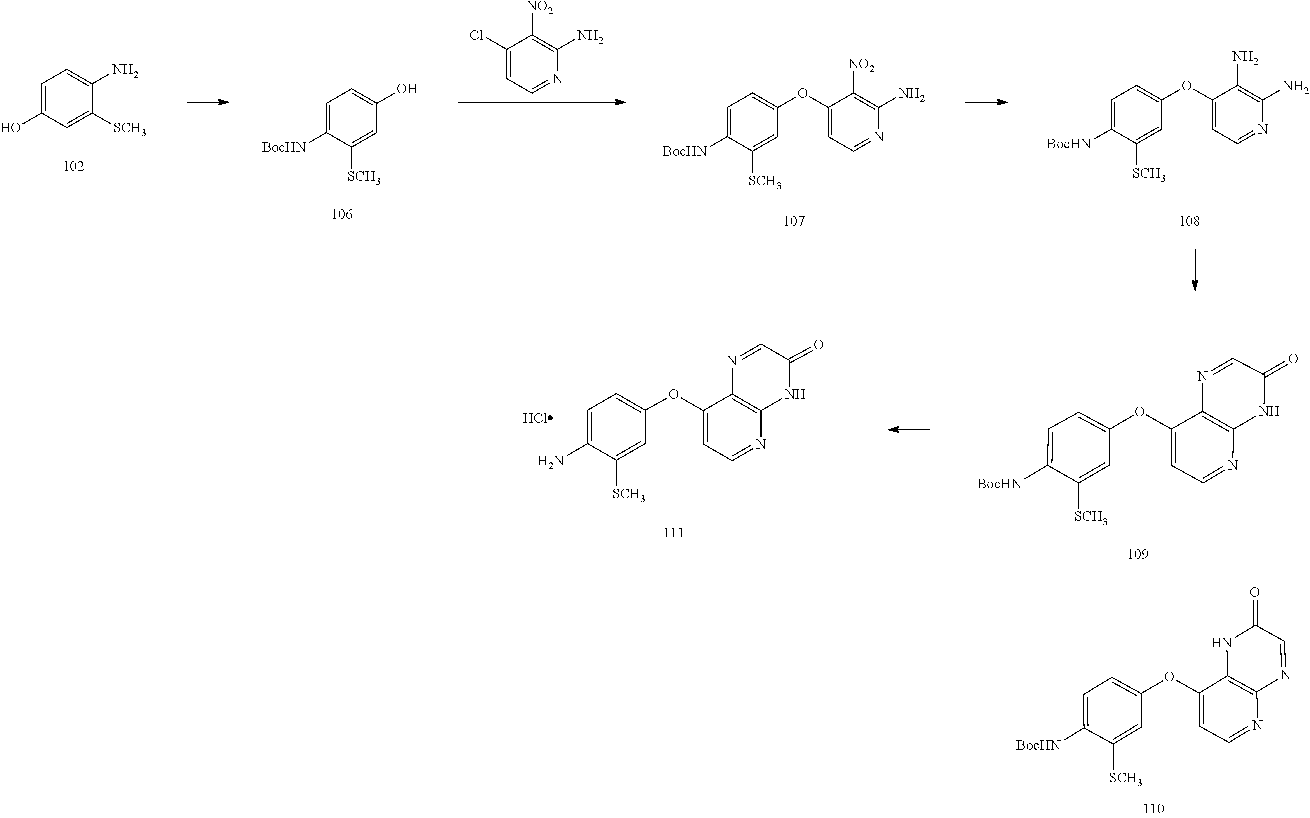

### Step 1. Synthesis of Compound (106)

4-amino-3-methylsulfanyl-phenol 102 (12.7 g, 81.8 mmol, 1.0 eq) was added to a molten mixture of di-tert-butyl dicarbonate (17.9 g, 81.8 mmol, 1.0 eq) and indium (III) chloride (181 mg, 0.82 mmol, 0.01 eq) at 35° C. The reaction mixture was stirred at 35° C. for 4 hours. After cooling down, the reaction mixture was directly deposed as liquid on silica gel to be purified by flash chromatography. After cooling down, the reaction mixture was directly deposed as liquid on silica gel to be purified by flash chromatography. Flash chromatography was performed using cyclohexane and ethyl acetate to afford expected compound 106 (Yield: 83%, 17.3 g) as off-white solid.

### Step 2. Synthesis of Compound (107)

To a solution of tert-butyl N-(4-hydroxy-2-methylsulfanyl-phenyl)carbamate 106 (15.0 g, 58.7 mmol, 1.0 eq) in 600 mL of DMF was added potassium tert-butylate (8.6 g, 76.9 mmol, 1.3 eq). After stirring 30 min at room temperature, 4-chloro-2-amino-3-nitropyridine (10.1 g, 58.2 mmol, 0.99 eq) was added and the mixture was heated at 80° C. for 3 h. After cooling down, the mixture was evaporated to dryness and ethyl acetate was added. The organic layer was washed with water, dried over magnesium sulfate, filtered and evaporated under vacuo. The residue was purified by flash chromatography on silica gel using cyclohexane and ethyl acetate to afford tert-butyl N-[4-[(2-amino-3-nitro-4-pyridyl)oxy]-2-methylsulfanyl-phenyl]carbamate 107 (Yield: 76%, 17.6 g) as yellow powder.

### Step 3. Synthesis of Compound (108)

Palladium on activated charcoal (around 4.0 g) was added to a solution of tert-butyl N-[4-[(2-amino-3-nitro-4-pyridyl)oxy]-2-methylsulfanyl-phenyl]carbamate 107 (17.6 g, 44.9 mmol, 1.0 eq) in a mixture of ethanol (650 mL) and dichloromethane (150 mL). The reaction mixture was hydrogenated at room temperature over 1 atmosphere of hydrogen for 20 h. The reaction mixture was filtrated through a short pad of celite and rinsed with ethyl acetate. The filtrate was evaporated to afford tert-butyl N-[4-[(2,3-diamino-4-pyridyl)oxy]-2-methylsulfanyl-phenyl]carbamate 108 as brown foam (17.3 g, quantitative yield).

### Step 4. Synthesis of Compound (109)

To a solution of tert-butyl N-[4-[(2,3-diamino-4-pyridyl)oxy]-2-methylsulfanyl-phenyl]carbamate 108 (16.3 g, 45.0 mmol, 1.0 eq) in 240 mL of methanol was added glyoxylic acid monohydrate (41.4 g, 449.7 mmol, 10.0 eq). The mixture was stirred at room temperature for 20 h. The off-white precipitate formed was filtered and washed with a small amount of methanol to afford tert-butyl N-[2-methylsulfanyl-4-[(3-oxo-4H-pyrido[2,3-b]pyrazin-8-yl)oxy]phenyl]carbamate 109 (Yield: 64%, 11.6 g). The undesired regioisomer 110 was contained in the filtrate.

### Step 5. Synthesis of Compound (111): 8-(4-amino-3-(methylthio)phenoxy)pyrido[2,3-b]pyrazin-3(4H)-one, Hydrochloride

Tert-butyl N-[2-methylsulfanyl-4-[(3-oxo-4H-pyrido[2,3-b]pyrazin-8-yl)oxy]phenyl]carbamate 109 (11.6 g, 29.0 mmol, 1.0 eq) was solubilized in 4N solution of hydrochloric acid in 1,4-dioxane (250 ml) and the reaction mixture was stirred at room temperature for 3 hours. The precipitate obtained was filtered and rinsed with dioxane and ethyl acetate to afford 8-(4-amino-3-methylsulfanyl-phenoxy)-4H-pyrido[2,3-b]pyrazin-3-one, hydrochloride 111 (12 g, quantitative yield) as beige solid.

### Synthesis of 117: 8-(4-amino-3-chlorophenoxy)pyrido[2,3-b]pyrazin-3(4H)-one Hydrochloride

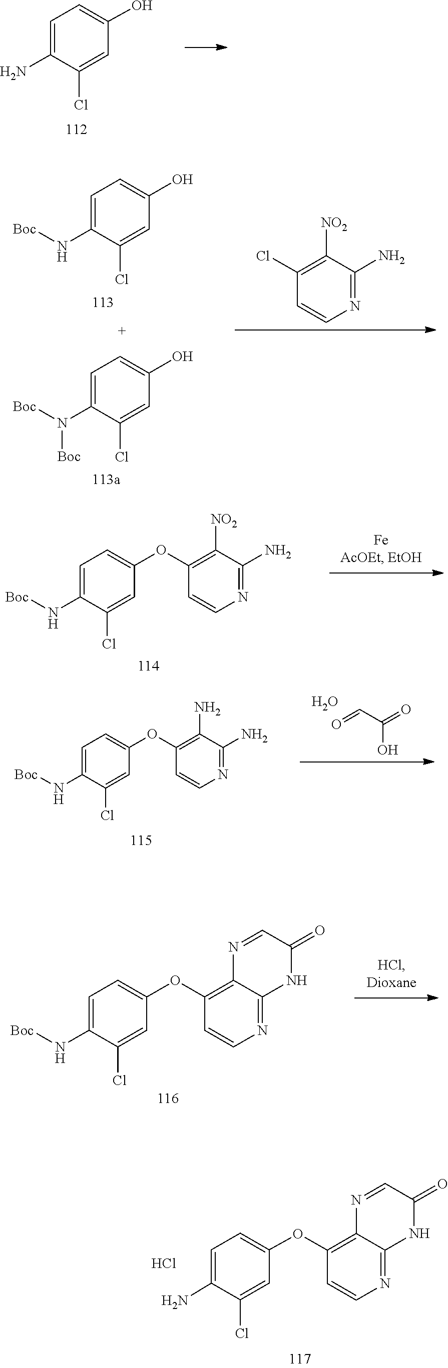

### Step 1. Synthesis of Compound (113)

4-Amino-3-chlorophenol 112 (3.0 g, 20.9 mmol, 1.0 eq) was added to a molten mixture of di-tert-butyl dicarbonate (4.6 g, 20.9 mmol, 1.0 eq) and indium (III) chloride (46 mg, 0.21 mmol, 0.01 eq). The reaction mixture was stirred at 35° C. for 4 hours. After cooling down, flash chromatography on silica gel was performed using dichloromethane and ethyl acetate to afford 113 (Yield: 9%, 480 mg) as yellow oil. Diprotected compound 113a was also isolated (790 mg) and used also in the next step.

### Step 2. Synthesis of Compound (114)

To a solution of the mixture containing tert-butyl N-(2-chloro-4-hydroxy-phenyl)carbamate 113 and by-product 113a (1.27 g, 4.27 mmol, 1.0 eq) in DMF (0.1M) was added potassium tert-butylate (623 mg, 5.5 mmol, 1.3 eq). After stirring 30 minutes 4-chloro-2-amino-3-nitropyridine (741 mg, 4.27 mmol, 1.0 eq) was added and the mixture was heated at 80° C., 3 hours. Flash chromatography on silica gel was performed using dichloromethane and ethyl acetate to afford expected compound 114 (Yield: 52%, 850 mg) as yellow powder.

### Step 3. Synthesis of Compound (115)

Tert-butyl N-[4-[(2-amino-3-nitro-4-pyridyl)oxy]-2-chloro-phenyl]carbamate 114 (850 mg, 2.23 mmol, 1.0 eq) was solubilized in acetic acid (20 mL) and ethanol (2 mL). Iron powder (374 mg, 6.7 mmol, 3.0 eq) was added and the reaction mixture was stirred at 45° C. for 24 h. After cooling down, the mixture was filtrated through a short pad of celite and rinsed with methanol. The filtrate was absorbed on silica gel to be purified by flash chromatography using ethyl acetate and methanol to afford the expected compound as colorless oil (Yield: 95%, 740 mg).

### Step 4. Synthesis of Compound (116)

Tert-butyl N-[4-[(2,3-diamino-4-pyridyl)oxy]-2-chloro-phenyl]carbamate 115 (740 mg, 2.11 mmol, 1.0 eq) in methanol was added dropwise to a solution of glyoxylic acid monohydrate (1.9 g, 21.1 mmol, 10.0 eq) in methanol. The mixture was stirred at room temperature for 2 days. The off-white precipitate formed was filtered and washed with a small amount of methanol to afford expected compound 116 (Yield: 54%, 446 mg).

### Step 5. Synthesis of Compound (117)

Tert-butyl N-[2-chloro-4-[(3-oxo-4H-pyrido[2,3-b]pyrazin-8-yl)oxy]phenyl]carbamate 116 (446 mg, 1.15 mmol, 1.0 eq) was put in 4N solution of hydrochloric acid in 1,4-dioxane (10 mL) and the reaction mixture was stirred at room temperature for 24 h and then evaporated to dryness to afford expected compound in mixture with 13% of starting material. The compound 117 was used without further purification in the next step.

### Synthesis of 123: 8-(4-amino-3-methylphenoxy)pyrido[2,3-b]pyrazin-3(4H)-one Hydrochloride

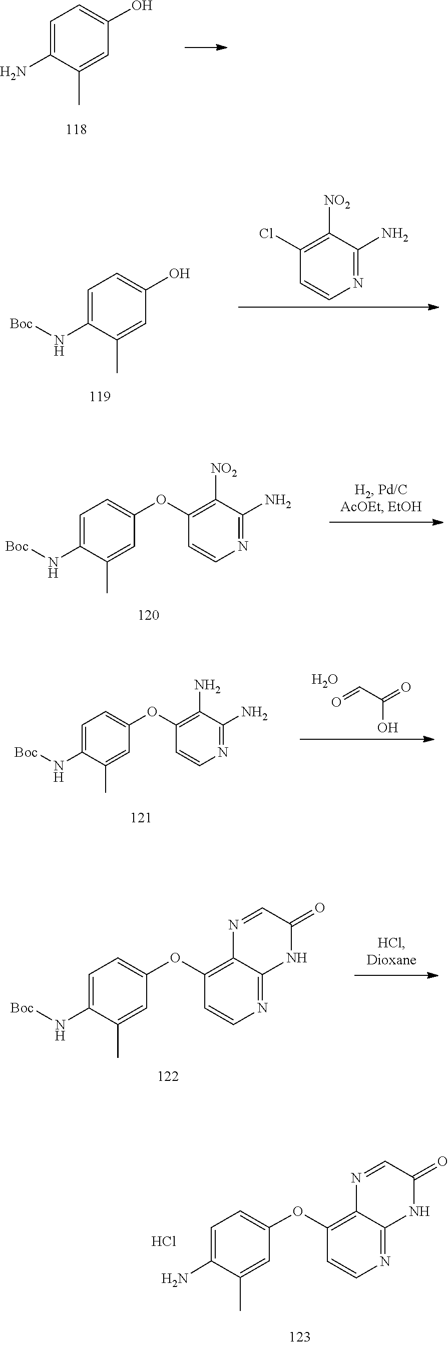

### Step 1. Synthesis of Compound (119)

4-Amino-3-methylphenol 118 (3.0 g, 24.3 mmol, 1.0 eq) was added to a molten mixture of di-tert-butyl dicarbonate (5.3 g, 24.3 mmol, 1.0 eq) and indium (111) chloride (54 mg, 0.24 mmol, 0.01 eq). The reaction mixture was stirred at 35° C. for 4 h. After cooling down, flash chromatography on silica gel was performed using dichloromethane and ethyl acetate to afford expected compound 119 (Yield: 98%, 5.3 g) as pale rose foam.

### Step 2. Synthesis of Compound (120)

To a solution of tert-butyl N-(2-methyl-4-hydroxy-phenyl)carbamate 119 (5.31 g, 23.78 mmol, 1.0 eq) in DMF (0.1M) was added potassium tert-butylate (3.47 g, 30.91 mmol, 1.3 eq). After stirring 30 minutes 4-chloro-2-amino-3-nitropyridine (4.13 g, 23.78 mmol, 1.0 eq) was added and the mixture was heated at 80° C., 3 hours. Flash chromatography on silica gel was performed using dichloromethane and ethyl acetate to afford expected compound 120 (Yield: 47%, 4.0 g) as yellow powder.

### Step 3. Synthesis of Compound (121)

Palladium on activated charcoal (around 500 mg) was added to a solution of tert-butyl N-[4-[(2-amino-3-nitro-4-pyridyl)oxy]-2-methyl-phenyl]carbamate 120 (4.0 g, 11.1 mmol, 1.0 eq) in a mixture of ethanol (200 mL) and ethyl acetate (100 mL). The reaction mixture was hydrogenated at room temperature over 1 atmosphere of hydrogen for 20 h. The reaction mixture was filtrated through a short pad of celite and rinsed with ethyl acetate and methanol. The filtrate was evaporated to dryness to afford the expected compound 121 as brown foam (3.7 g, quantitative yield). The compound was used directly in the next step without further purification.

### Step 4. Synthesis of Compound (122)

Tert-butyl N-[4-[(2,3-diamino-4-pyridyl)oxy]-2-methyl-phenyl]carbamate 121 (1.0 g, 3.03 mmol, 1.0 eq)) in methanol was added dropwise to a solution of glyoxylic acid monohydrate (1.4 g, 15.15 mmol, 5.0 eq) in methanol. The mixture was stirred at room temperature for 2 days. The off-white precipitate formed was filtered and washed with a small amount of methanol to afford expected compound 122 (Yield: 26%, 288 mg).

### Step 5. Synthesis of Compound (123)

Tert-butyl N-[2-methyl-4-[(3-oxo-4H-pyrido[2,3-b]pyrazin-8-yl)oxy]phenyl]carbamate 122 (288 mg, 0.78 mmol, 1.0 eq) was put in 4N solution of hydrochloric acid in 1,4-dioxane (6.5 mL) and the reaction mixture was stirred at room temperature for 24 h and then evaporated to dryness to afford expected compound as hydrochloride salt as beige powder. The compound 123 was used without further purification in the next step.

### Synthesis of 130: 8-(4-amino-3-(ethylthio)phenoxy)pyrido[2,3-b]pyrazin-3(4H)-one Hydrochloride

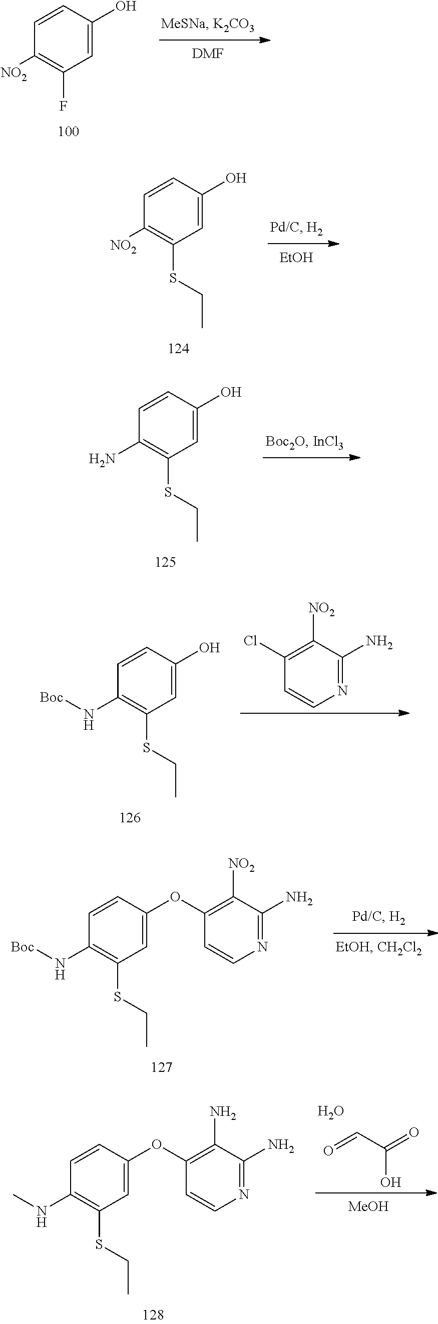

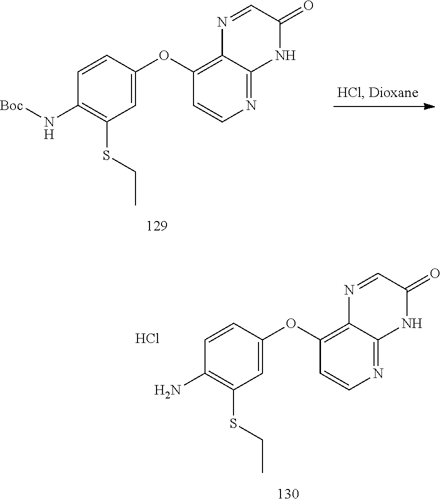 [SMILES: CCSc1cc(Oc2ccnc3[nH]c(=O)cnc23)ccc1NC(=O)OC(C)(C)C ; Cl ; CCSc1cc(Oc2ccnc3[nH]c(=O)cnc23)ccc1N]

### Step 1. Synthesis of Compound (124)

To a solution of 3-fluoro-4-nitrophenol 100 (9.3 g, 59.4 mmol, 1.0 eq) in dry DMF (350 mL) were added portionwise sodium ethanethiolate (10.0 g, 118.9 mmol, 2.0 eq) and potassium carbonate (24.6 g, 178.3 mmol, 3.0 eq). The mixture was stirred at room temperature for 20 h. The suspension was filtrated on a short pad of celite and rinsed with ethyl acetate. The filtrate was concentrated. Water was added, the aqueous phase was acidified to pH 4 with a 1 N solution of hydrochloric acid and extracted with ethyl acetate. The organic layers were dried over magnesium sulfate, filtered and evaporated under vacuo to afford expected compound 124 (Yield: 88%, 9.6 g) as yellow solid.

### Step 2. Synthesis of Compound (125)

Iron powder (7.1 g, 128.0 mmol, 3.0 eq) was added to a solution of 3-ethylsulfanyl-4-nitro-phenol 124 (8.5 g, 42.7 mmol, 1.0 eq) in acetic acid (200 mL) and ethanol (20 mL). The reaction mixture was stirred at room temperature for 48 h. The reaction mixture was filtrated through a short pad of celite, rinsed with ethanol and concentrated. The residue was purified by flash chromatography over silica gel using cyclohexane and ethyl acetate to afford the expected compound 125 as grey solid (Yield: 53%, 3.8 g).

### Step 3. Synthesis of Compound (126)

4-amino-3-ethylsulfanyl-phenol 125 (3.8 g, 22.5 mmol, 1.0 eq) was added to a molten mixture of di-tert-butyl dicarbonate (4.9 g, 22.5 mmol, 1.0 eq) and indium (III) chloride (50 mg, 0.02 mmol, 0.01 eq). The reaction mixture was stirred at 35° C. for 4 hours. After cooling down, flash chromatography on silica gel was performed using cyclohexane and ethyl acetate to afford expected compound 126 (Yield: 58%, 3.5 g) as orange oil.

### Step 4. Synthesis of Compound (127)

To a solution of tert-butyl N-(4-hydroxy-2-ethylsulfanyl-phenyl)carbamate 126 (3.5 g, 13.0 mmol, 1.0 eq) in DMF (0.1M) was added potassium tert-butylate (1.9 g, 16.9 mmol, 1.3 eq). After stirring 30 minutes 4-chloro-2-amino-3-nitropyridine (2.2 g, 12.9 mmol, 0.99 eq) was added and the mixture was heated at 80° C., 3 hours. Flash chromatography on silica gel was performed using cyclohexane and ethyl acetate to afford expected compound 127 (Yield: 74%, 3.9 g) as orange foam.

### Step 5. Synthesis of Compound (128)

Palladium on activated charcoal (around 1 g) was added to a solution of tert-butyl N-[4-[(2-amino-3-nitro-4-pyridyl)oxy]-2-ethylsulfanyl-phenyl]carbamate 127 (3 g, 9.6 mmol, 1.0 eq) in ethanol (60 ml). The reaction mixture was hydrogenated at room temperature over 1 atmosphere of hydrogen for 2 days. The reaction mixture was filtrated through a short pad of celite and rinsed with ethanol. The filtrate was evaporated to afford the expected compound 128 as brown oil (Yield: 86%, 3.47 g).

### Step 6. Synthesis of Compound (129)

Tert-butyl N-[4-[(2,3-diamino-4-pyridyl)oxy]-2-ethylsulfanyl-phenyl]carbamate 128 (1.0 g, 2.66 mmol, 1.0 eq) in methanol was added dropwise to a solution of glyoxylic acid monohydrate (1.2 g, 13.3 mmol, 5.0 eq) in methanol. The mixture was stirred at room temperature for 2 days. The off-white precipitate formed was filtered and washed with a small amount of methanol to afford expected compound 129 (Yield: 34%, 378 mg).

### Step 7. Synthesis of Compound (130)

Tert-butyl N-[2-ethylsulfanyl-4-[(3-oxo-4H-pyrido[2,3-b]pyrazin-8-yl)oxy]phenyl]carbamate 129 (370 mg, 0.89 mmol, 1.0 eq) was put in 4N solution of hydrochloric acid in 1,4-dioxane (7.5 mL) and the reaction mixture was stirred at room temperature for 3 h and then evaporated to dryness to afford expected compound 130 as hydrochloride salt as beige solid (368 mg, quantitative yield). The compound was used without further purification in the next step.

### Synthesis of 132: 5-(4-amino-3-(methylthio)phenoxy)-3,4-dihydro-1,8-naphthyridin-2(1H)-one

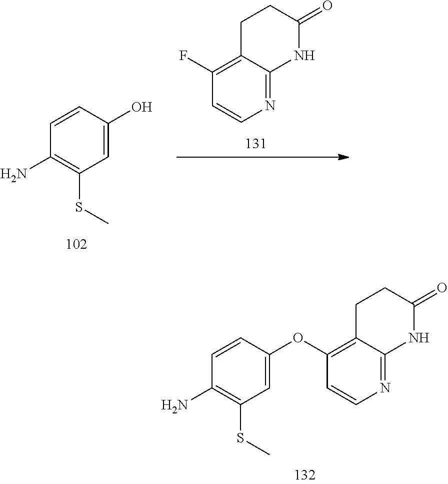

In a sealed tube, tBuOK (159 mg, 1.41 mmol) was added at room temperature to a solution of 4-amino-3-methylsulfanyl-phenol 102 (200 mg, 1.28 mmol) and 5-fluoro-3,4-dihydro-1H-1,8-naphthyridin-2-one 131 (214 mg, 1.28 mmol) in dry DMF (4 mL). The reaction mixture was stirred at 120° C. for 1 h. After cooling to room temperature, water (50 mL), aq. sat. NH₄Cl (50 mL) and AcOEt (100 mL) were added. The aqueous layer was extracted with AcOEt (2×30 mL). The combined organic fractions were dried over MgSO₄, filtered and concentrated in vacuo. The crude obtained was purified by flash chromatography (40 g, SiO₂, cyclohexane/AcOEt 100:0→0:100, 10 CV and AcOEt/CH₃OH 100:0→95:5, 5 CV) leading to the expected product 132 (Yield: 39%, 150 mg) as a brown powder.

### Synthesis of 136: 4-(3-(methylthio)-4-nitrophenoxy)pyridin-2-amine

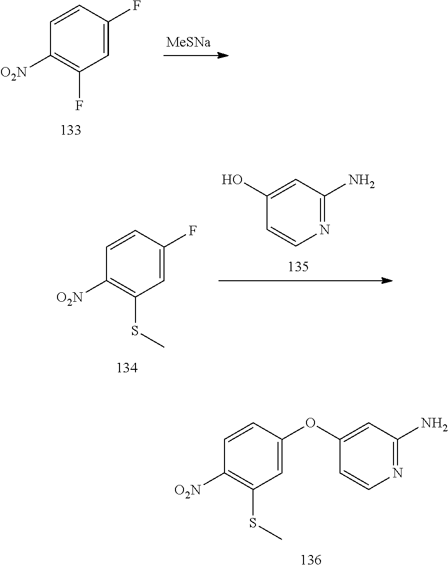 [SMILES: "MeSNa" ; O=[N+]([O-])c1ccc(F)cc1F ; CSc1cc(F)ccc1[N+](=O)[O-] ; Nc1cc(O)ccn1 ; CSc1cc(Oc2ccnc(N)c2)ccc1[N+](=O)[O-]]

### Step 1. Synthesis of Compound (134)

At 0° C., sodium thiomethoxide (4.4 g, 62.9 mmol) was added to a solution of 2,4-difluoro-1-nitro-benzene 133 (6.9 ml, 62.9 mmol) in dry pyridine (60 mL). The reaction mixture was stirred at 0° C. for 3 h, quenched with cold water (250 mL), and extracted with dichloromethane (2×60 mL). The combined organic fractions were dried over MgSO₄, filtered and concentrated in vacuo (azeotrope with toluene to remove residual pyridine). The crude obtained was purified by flash chromatography (3×340 g, SiO₂, cyclohexane/AcOEt 100:0→90:10, 10 CV) to furnish the expected product 134 (Yield: 39%, 4.6 g) as a yellow solid.

### Step 2. Synthesis of Compound (136)

In a sealed tube, DBU (2.9 ml, 19.2 mmol) was added at room temperature to a solution 2-aminopyridin-4-ol 135 (1.5 g, 13.9 mmol) in dry acetonitrile (0.1M). The mixture was stirred at room temperature for 30 min before addition of 4-fluoro-2-methylsulfanyl-1-nitro-benzene 134 (2.0 g, 10.7 mmol). The reaction mixture was stirred at 80° C. for 2 h30. After cooling to room temperature the crude obtained was triturated in AcOEt, filtrated off, and dried in vacuo to furnish the expected product (660 mg, 22%) as a yellow powder. The filtrate was purified by flash chromatography (120 g, SiO₂, cyclohexane/AcOEt 100:0→0:100, 10 CV and AcOEt/CH₃OH 100:0→90:10, 5 CV) leading to a new batch of expected product (Yield: 51%, 1.5 g) as a yellow solid.

### Synthesis of 140: 5-(4-amino-3-(methylthio)phenoxy)-N-methylpicolinamide

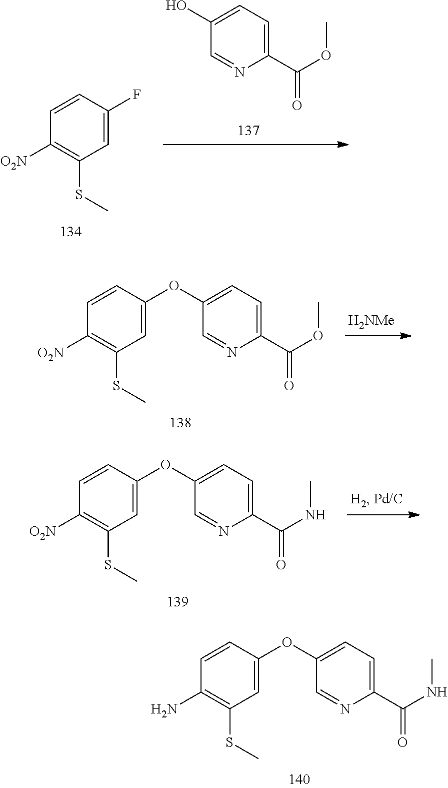

### Step 1. Synthesis of Compound (138)

In a sealed tube, DBU (2.9 ml, 19.2 mmol), methyl-5-hydroxypyridine-2-carboxylate 137 (319 mg, 2.08 mmol) in dry acetonitrile (0.1M). The mixture was stirred at room temperature for 30 min before addition of 4-fluoro-2-methylsulfanyl-1-nitro-benzene 134 (300 mg, 1.60 mmol). The reaction mixture was stirred at 80° C. for 5 h. After cooling to room temperature the suspension was filtered off and the powder was purified by flash chromatography (40 g, SiO₂, cyclohexane/CH₂Cl₂ 100:0→0:100, 10 CV and CH₂Cl₂/AcOEt 100:0→80:20, 5 CV) leading to the expected product 138 (Yield: 71%, 362 mg) as a yellow solid.

### Step 2. Synthesis of Compound (139)

In a sealed tube, a solution of methyl-5-(3-methylsulfanyl-4-nitro-phenoxy)pyridin-2-carboxylate 138 (362 mg, 1.13 mmol) and methylamine 2M in methanol (11.0 ml, 22.0 mmol) was heated at 100° C. for 4 h. After cooling to room temperature and concentration under vacuum, the crude obtained was purified by flash chromatography (40 g, SiO₂, dichloromethane/AcOEt 100:0→80:20, 10 CV) leading to the expected product 139 (Yield: 97%, 352 mg) as a yellow powder.

### Step 3. Synthesis of Compound (140)

A suspension of methyl-5-(3-methylsulfanyl-4-nitro-phenoxy)pyridin-2-carbamide 139 (352 mg, 1.10 mmol) and Pd/C (20 mg) in ethanol (20 ml) was stirred under hydrogen (1 atm) at room temperature. After 18 h at room temperature and work-up, the expected product 140 (Yield: 35%, 110 mg) was recovered.

### Synthesis of 142: N-(4-(4-amino-3-(methylthio)phenoxy)pyridin-2-yl)acetamide

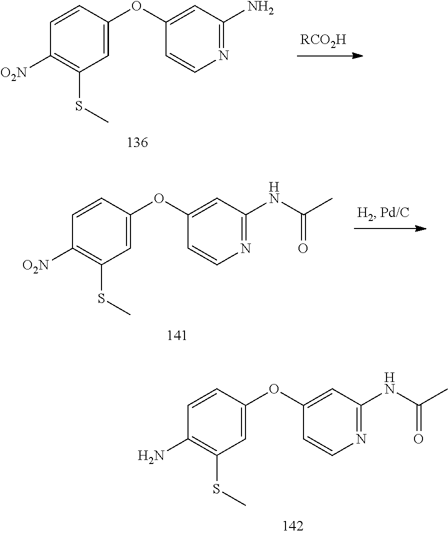

### Step 1. Synthesis of Compound (141)

To a stirred solution of 4-(3-methylsulfanyl-4-nitro-phenoxy)pyridin-2-amine 136 (320 mg, 1.15 mmol) in anhydrous dichloromethane (5 mL) and DIEA (0.40 ml, 2.30 mmol), cooled at 0° C., was added acetyl chloride (0.13 ml, 1.74 mmol). The mixture was allowed to warm to room temperature and stirred at this temperature for 1 h. NH₄Cl sat (30 mL) was added. The aqueous layer was extracted with dichloromethane (2×30 mL). The combined organic fractions were dried over MgSO₄, filtered and concentrated in vacuo. The crude obtained was purified by flash chromatography (40 g, SiO₂, cyclohexane/AcOEt 100:0→20:80, 10 CV) leading to the expected product 141 (Yield: 81%, 300 mg) as a yellow solid.

### Step 2. Synthesis of Compound (142)

A suspension of N-[4-(3-methylsulfanyl-4-nitro-phenoxy)-2-pyridyl]acetamide 141 (300 mg, 0.94 mmol) and Pd/C (50 mg) in ethanol (20 mL) was stirred under hydrogen (1 atm) at room temperature. After 18 h at room temperature and work-up, the expected product 142 (Yield: 50%, 136 mg) was recovered as a yellow solid.

### Synthesis of 144: 8-(4-amino-3-(methylthio)phenoxy)-4-methylpyrido[2,3-b]pyrazin-3(4H)-one Hydrochloride

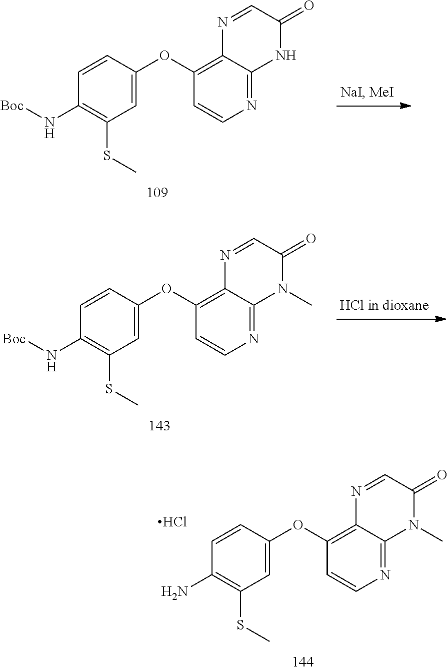

### Step 1. Synthesis of Compound (143)

At 0° C., sodium hydride (60% wt., 44 mg, 1.10 mmol) was added to a suspension of tert-butyl N-{2-methylsulfanyl-4-[(3-oxo-4H-pyrido[2,3-b]pyrazin-8-yl)oxy]phenyl}carbamate 109 (400 mg, 1.00 mmol) in dry DMF (10 mL). After 30 min at 0° C., methyl iodide (0.62 mL, 10 mmol) was added. The reaction mixture was stirred at room temperature for 3 h, quenched with water (60 mL), and extracted with AcOEt (3×30 mL). The combined organic fractions were washed with brine (40 mL), dried over MgSO₄, filtered and concentrated in vacuo. The crude obtained was purified by flash chromatography (40 g, SiO₂, cyclohexane/AcOEt 100:0→50:50, 10 CV) leading to the expected product 143 (Yield: 64%, 265 mg).

### Step 1. Synthesis of Compound (144)

Tert-butyl N-[4-(4-methyl-3-oxo-1,3-pyrido[2,3-b]pyrazin-8-yl)oxy-2-methylsulfanyl-phenyl] carbamate (265 mg, 0.63 mmol) was treated with 4N HCl in dioxane (5 ml) and the reaction mixture was stirred at room temperature for 18 h. The suspension obtained was filtered off, washed with dioxane, and dried under vacuum. The pale pink solid recovered 144 (222 mg, quantitative yield) was directly used for next step without any purification.

### Synthesis of 149: 8-(4-amino-3-(trifluoromethyl)phenoxy)pyrido[2,3-b]pyrazin-3(4H)-one Hydrochloride

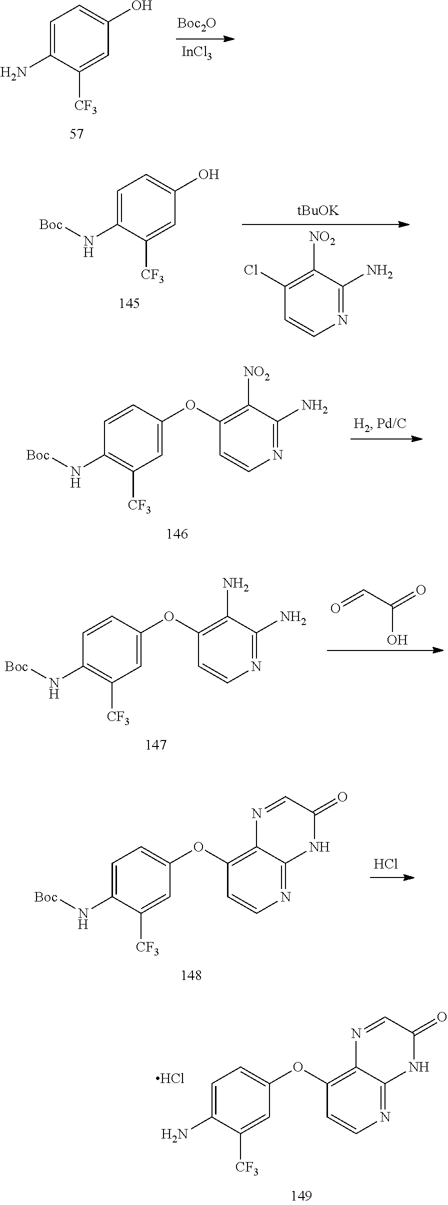

### Step 1. Synthesis of Compound (145)

4-amino-3-(trifluoromethyl)phenol 57 (14.5 g, 81.8 mmol, 1.0 eq) was added to a molten mixture of di-tert-butyl dicarbonate (17.9 g, 81.8 mmol, 1.0 eq) and indium (III) chloride (181 mg, 0.82 mmol, 0.01 eq). The reaction mixture was stirred at 35° C. for 4 hours. After cooling down, flash chromatography on silica gel was performed using cyclohexane and ethyl acetate to afford expected compound 145 (Yield: 83%, 18.8 g) as off-white solid.

### Step 2. Synthesis of Compound (146)

Potassium tert-butoxide (2.2 g, 19.2 mmol) was added at room temperature to a solution of tert-butyl N-[4-hydroxy-2-(trifluoromethyl)phenyl]carbamate 145 (4.1 g, 14.8 mmol) in DMF (195 mL). After 15 min, 4-chloro-3-nitro-pyridin-2-amine (2.5 g, 14.6 mmol) was added. The reaction mixture was heated at 80° C. for 3 h. After cooling at room temperature and evaporation of the solvent under vacuum, the crude obtained was diluted in AcOEt and washed with water. The organic layer was dried over MgSO₄, filtered and concentrated in vacuo. The residue obtained was purified by flash chromatography (80 g, SiO₂, CH₂Cl₂/AcOEt 100:0→85:15, 10 CV) leading to the expected product 146 (Yield: 9%, 534 mg) as a yellow powder.

### Step 3. Synthesis of Compound (147)

Palladium on activated charcoal (around 50 mg) was added to a solution of tert-butyl N-{4-[(2-amino-3-nitro-4-pyridyl)oxy]-2-(trifluoromethyl) phenyl}carbamate 146 (530 mg, 1.29 mmol) in ethanol (60 mL) and dichloromethane (10 mL). The reaction mixture was hydrogenated at room temperature over 1 atmosphere of hydrogen (1 atm). After 18 h at room temperature and work-up, the expected product 147 (Yield: 96%, 480 mg) was recovered as a brown foam.

### Step 4. Synthesis of Compound (148)

Tert-butyl N-{4-[(2,3-diamino-4-pyridyl)oxy]-2-(trifluoromethyl)phenyl}carbamate 147 (480 mg, 1.24 mmol) in methanol was added dropwise to a solution of glyoxylic acid (3.9 g, 42 mmol) in methanol (23 mL). The mixture was stirred at room temperature for 18 h. After work-up, the expected product 148 (Yield: 40%, 208 mg) was recovered as a white powder.

### Step 5. Synthesis of Compound (149)

Tert-butyl N-{4-[(3-oxo-4H-pyrido[2,3-b]pyrazin-8-yl)oxy]-2-(trifluoromethyl)phenyl} carbamate 148 (208 mg, 0.49 mmol) was treated with HCl 4N in dioxane (4 mL) and the reaction mixture was stirred at room temperature for 3 h. After concentration under vacuum, the gum recovered (180 mg) was directly used for next step without any purification.

### Synthesis of 155: 8-((4-aminonaphthalen-1-yl)oxy)pyrido[2,3-b]pyrazin-3(4H)-one hydrochloride

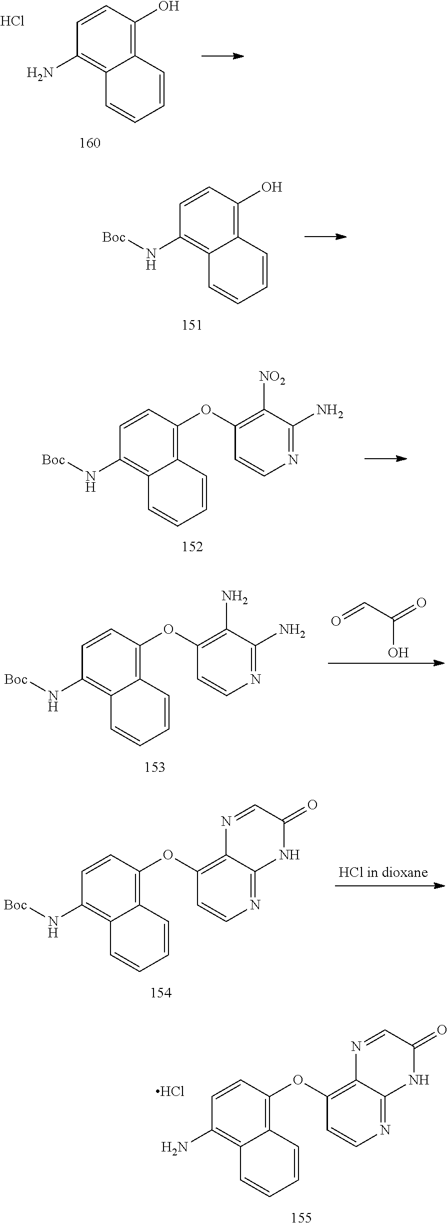

### Step 1. Synthesis of Compound (151)

To a solution of 4-aminonaphthol hydrochloride 150 (10.0 g, 51.1 mmol, 1.0 eq) in 75 mL of THF were added di-tert-butyl dicarbonate (12.3 g, 56.2 mmol, 1.1 eq) and lithium hydroxide monohydrate (2.1 g, 51.1 mmol, 1.0 eq). The reaction mixture was stirred at room temperature for 18 h. THF was concentrated and water was added. The solution was extracted with ethyl acetate. The organic phases were combined, dried over magnesium sulfate, filtered and evaporated. The residue was purified by flash chromatography over silica gel using cyclohexane and ethyl acetate to afford expected compound as purple powder (Yield: 79%, 10.5 g).

### Step 2. Synthesis of Compound (152)

To a solution of tert-butyl N-(4-hydroxy-1-naphthyl)carbamate 151 (6.0 g, 23.13 mmol, 1.0 eq) in 280 mL of DMF was added potassium tert-butylate (3.4 g, 30.03 mmol, 1.3 eq). After stirring 30 min at room temperature, 4-chloro-2-amino-3-nitropyridine (0.99 eq) was added and the mixture was heated at 80° C. for 3 h. After cooling down, the mixture was evaporated to dryness and ethyl acetate and water were added. The precipitate obtained was filtered, washed with ethyl acetate and water and dried to afford expected compound 152 as yellow powder (Yield: 73%, 6.75 g).

### Step 3. Synthesis of Compound (153)

Palladium on activated charcoal was added to a solution of tert-butyl N-[4-[(2-amino-3-nitro-4-pyridyl)oxy]-1-naphthyl]carbamate 152 (6.6 g, 16.6 mmol, 1.0 eq) in ethanol and dichloromethane. The reaction mixture was hydrogenated at room temperature over 1 bar of hydrogen for 20 h. The reaction mixture was filtrated through a short pad of celite and rinsed with methanol, dichloromethane and ethyl acetate. The filtrate was evaporated to afford the expected compound 153 as brown powder (Yield: 99%, 6.1 g).

### Step 4. Synthesis of Compound (154)

Trans tert-butyl N-{4-[(2,3-diamino-4-pyridyl)oxy]cyclohexyl}carbamate 153 (6.1 g, 16.53 mmol) in methanol was added dropwise to a solution of glyoxylic acid (15.2 g, 0.16 mmol) in methanol (100 mL). The mixture was stirred at room temperature for 20 h. After work-up, the expected product 154 (Yield: 60%, 4.0 g) was recovered as a beige powder.

### Step 5. Synthesis of Compound (155)

Tert-butyl N-{4-[(3-oxo-4H-pyrido[2,3-b]pyrazin-8-yl)oxy]-1-naphthyl} carbamate 154 (2.0 g, 4.94 mmol) was treated with HCl 4N in dioxane (12 mL, 49 mmol, 10.0 eq) and the reaction mixture was stirred at room temperature for 1 h. The suspension obtained was filtered off, washed with dioxane, and dried under vacuum. The brown powder recovered (1.6 g, quantitative) was directly used for next step without any purification.

### Synthesis of 157: 2,2,2-trichloroethyl (5-(tert-butyl)thiazol-2-yl)carbamate

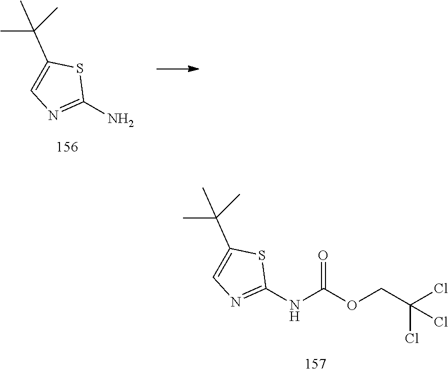

At 0° C., to a solution of 5-tert-butylthiazol-2-yl)amine 156 (500 mg, 3.2 mmol, 1.0 eq) in dry THF (0.2M) was added dropwise dry pyridine (1.1 eq) followed by 2,2,2-trichloroethylchloroformate (0.48 mL, 3.52 mmol, 1.1 eq). The reaction mixture was stirred at room temperature for 18 h. Water was added, and the reaction mixture was extracted with ethyl acetate. The organic layers were combined, dried over magnesium sulfate, filtered and evaporated. The compound 157 was obtained as white powder (Yield: 94%, 1.0 g) and was used without purification in the next step.

### Synthesis of 158: 2,2,2-trichloroethyl (3-(tert-butyl)-1-phenyl-1H-pyrazol-5-yl)carbamate

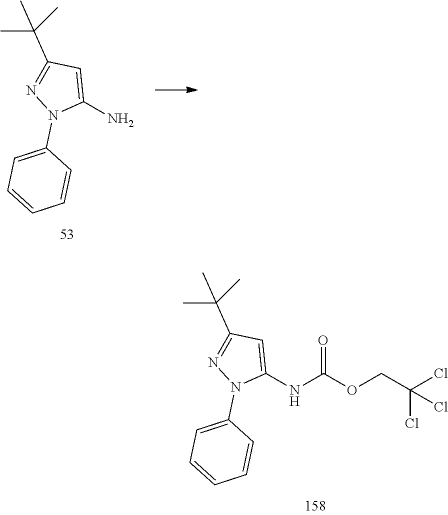 [SMILES: CC(C)(C)c1cc(N)n(-c2ccccc2)n1 ; CC(C)(C)c1cc(NC(=O)OCC(Cl)(Cl)Cl)n(-c2ccccc2)n1]

At 0° C., to a solution of 3-(tert-butyl)-1-phenyl-1H-pyrazol-5-amine 53 (690 mg, 3.2 mmol, 1.0 eq) in dry THF (0.2M) was added dropwise dry pyridine (1.1 eq) followed by 2,2,2-trichloroethylchloroformate (0.48 mL, 3.52 mmol, 1.1 eq). The reaction mixture was stirred at room temperature for 18 h. Water was added, and the reaction mixture was extracted with ethyl acetate. The organic layers were combined, dried over magnesium sulfate, filtered and evaporated. The compound 158 was obtained as white powder (Yield: 95%, 1.19 g) and was used without purification in the next step.

### Synthesis of 164: 8-(4-amino-3-methoxyphenoxy)pyrido[2,3-b]pyrazin-3(4H)-one Hydrochloride

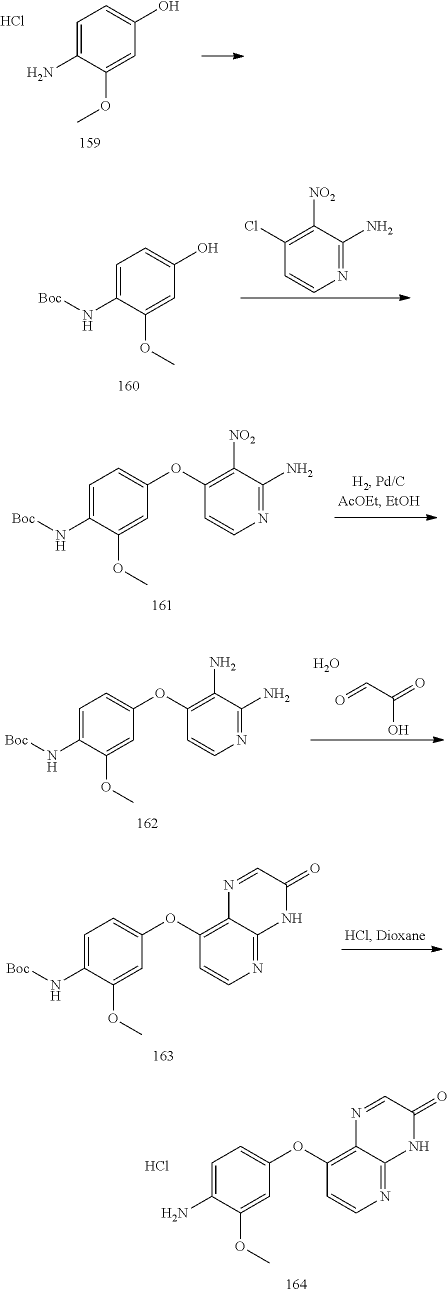

### Step 1. Synthesis of Compound (160)

4-Amino-3-methoxyphenol hydrochloride 159 (1.0 g, 5.69 mmol, 1.0 q) was put in suspension in THF (20 mL). Triethylamine (1.6 mL, 11.38 mmol, 2.0 eq) and di-tert-butyl dicarbonate (1.24 g, 5.69 mmol, 1.0 eq) were added and the mixture was stirred for 2 days at reflux. After cooling down, the mixture was adsorbed on silica gel and purified by flash chromatography using cyclohexane and ethyl acetate to afford expected compound 160 (Yield: 90%, 1.23 g) as brown oil.

### Step 2. Synthesis of Compound (161)

Tert-butyl N-(2-methoxy-4-hydroxy-phenyl)carbamate 160 (1.23 g, 5.14 mmol, 1.0 eq) in 70 mL of DMF was added potassium tert-butylate (750 mg, 6.68 mmol, 1.3 eq). After stirring 30 min at room temperature, 4-chloro-2-amino-3-nitropyridine (892 mg, 5.14 mmol, 1.0 eq) was added and the mixture was heated at 80° C. for 3 h. The crude was purified by flash chromatography on silica gel using dichloromethane and ethyl acetate to afford expected compound (Yield: 38%, 730 mg) as orange powder.

### Step 3. Synthesis of Compound (162)

Palladium on activated charcoal (around 100 mg) was added to a solution of tert-butyl N-[4-[(2-amino-3-nitro-4-pyridyl)oxy]-2-methoxy-phenyl]carbamate 161 (730 mg, 1.94 mmol, 1.0 eq) in a mixture of ethanol (40 mL) and ethyl acetate (20 mL). The reaction mixture was hydrogenated at room temperature over 1 atmosphere of hydrogen for 2 days. The reaction mixture was filtrated through a short pad of celite and rinsed with ethyl acetate. The filtrate was evaporated to dryness to afford the expected compound 162 as dark brown solid (672 mg, quantitative yield). The compound was used directly in the next step without further purification.

### Step 4. Synthesis of Compound (163)

Tert-butyl N-[4-[(2,3-diamino-4-pyridyl)oxy]-2-methoxy-phenyl]carbamate 162 (672 mg, 1.94 mmol, 1.0 eq)) in methanol was added dropwise to a solution of glyoxylic acid (1.8 g, 19.4 mmol, 10.0 eq) in methanol (15 mL). The mixture was stirred at room temperature for 18 h. After work-up, the expected product 163 (Yield: 54%, 400 mg) was obtained as a beige powder after filtration.

### Step 5. Synthesis of Compound (164)

Tert-butyl N-[2-methoxy-4-[(3-oxo-4H-pyrido[2,3-b]pyrazin-8-yl)oxy]phenyl]carbamate 163 (400 mg, 1.04 mmol, 1.0 eq) was treated with HCl 4N in dioxane (8 mL) and the reaction mixture was stirred at room temperature for 2 h. The precipitate was filtered, washed with diethyl ether and dried under vacuo to afford expected compound 164 as hydrochloride salt as brown solid (300 mg, quantitative yield).

### Synthesis of 166: 3-(tert-butyl)-1-(6-methylpyridin-3-yl)-1H-pyrazol-5-amine

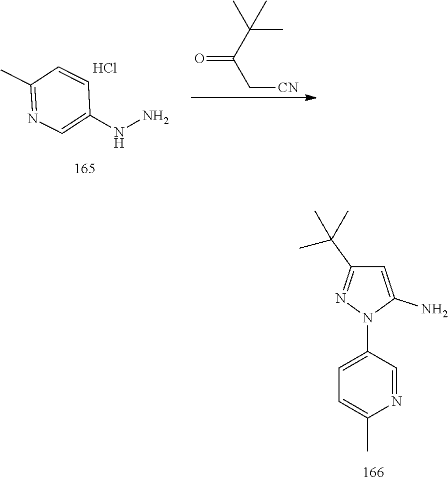

[6-methyl-3-pyridyl]hydrazine hydrochloride 165 (500 mg, 3.13 mmol, 1.0 eq) and pivaloyl acetonitrile (437 mg, 3.5 mmol, 1.6 eq)) in ethanol wad added hydrochloric acid 12 N (10.0 eq). The mixture was stirred at reflux for 20 hours. After cooling down, sodium hydrogencarbonate solid was added until pH was around 7. The residual salts were filtrated and washed with methanol. The filtrate was purified by flash chromatography on silica gel using 100% of ethyl acetate to afford expected compound 166 as yellow oil (Yield: 62%, 447 mg).

### Synthesis of 167: 4-((4-aminonaphthalen-1-yl)oxy)-N-methylpicolinamide

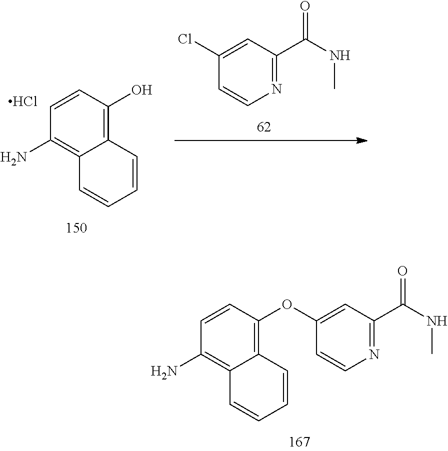 [SMILES: Cl ; Nc1ccc(O)c2ccccc12 ; CNC(=O)c1cc(Cl)ccn1 ; CNC(=O)c1cc(Oc2ccc(N)c3ccccc23)ccn1]

In a sealed tube, tBuOK (660 mg, 5.87 mmol) followed by 4-chloro-N-methyl-pyridine-2-carboxamide 62 (436 mg, 2.55 mmol) were added at room temperature to a solution of 4-amino-1-naphthol hydrochloride 150 (500 mg, 2.55 mmol) in dry DMA (7 mL). The reaction mixture was stirred at 100° C. for 20 h. After cooling to room temperature, water (50 mL), aq. sat. NH₄Cl (20 mL) and AcOEt (50 mL) were added. The aqueous layer was extracted with AcOEt (2×30 mL). The combined organic fractions were dried over MgSO₄, filtered and concentrated in vacuo. The crude obtained was purified by flash chromatography (80 g, SiO₂, cyclohexane/AcOEt 100:0→0:100, 10 CV) leading to the expected product 167 (Yield: 29%, 216 mg) as a dark oil.

### Synthesis of 169: 3-(tert-butyl)-1-(2-(dimethylamino)ethyl)-1H-pyrazol-5-amine

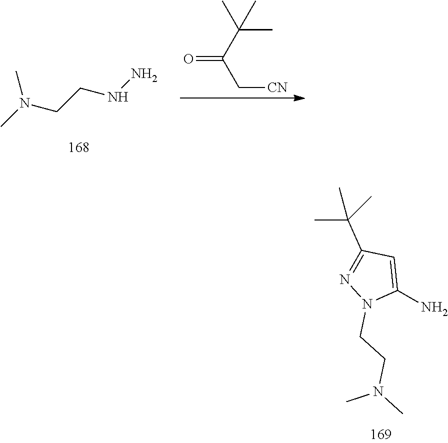

[2-(dimethylamino)ethyl]hydrazine 168 (500 mg, 4.84 mmol, 1.0 eq) and pivaloyl acetonitrile (970 mg, 7.75 mmol, 1.6 eq) in ethanol wad added hydrochloric acid 12 N (10.0 eq). The mixture was stirred at reflux for 18 h. After cooling down, sodium hydrogencarbonate solid was added until pH was around 7. The residual salts were filtrated and washed with methanol. The filtrate was purified by flash chromatography on silica gel using dichloromethane and methanol to afford expected compound 169 as orange solidified oil (Yield: 96%, 980 mg).

### Synthesis of 171

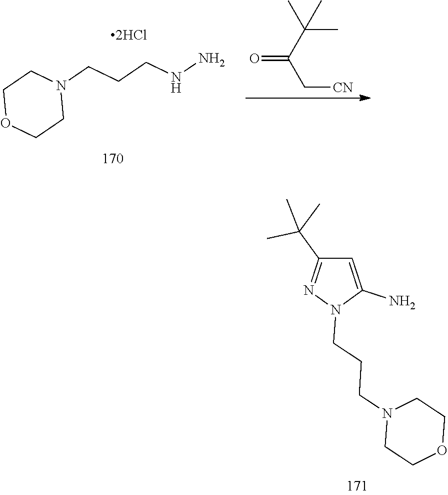

[3-morpholinopropyl]hydrazine dihydrochloride 170 (500 mg, 2.15 mmol, 1.0 eq) and pivaloyl acetonitrile (431 mg, 3.44 mmol, 1.6 eq) in ethanol wad added hydrochloric acid 12 N (10.0 eq). The mixture was stirred at reflux for 18 h. After cooling down, sodium hydrogencarbonate solid was added until pH was around 7. The residual salts were filtrated and washed with methanol. The filtrate was purified by flash chromatography on silica gel using dichloromethane and methanol to afford expected compound 171 as light yellow oil (Yield: 66%, 381 mg, 66%).

### Synthesis of 173: Tert-Butyl (4-(4-amino-3-(methylthio)phenoxy)pyridin-2-yl)carbamate

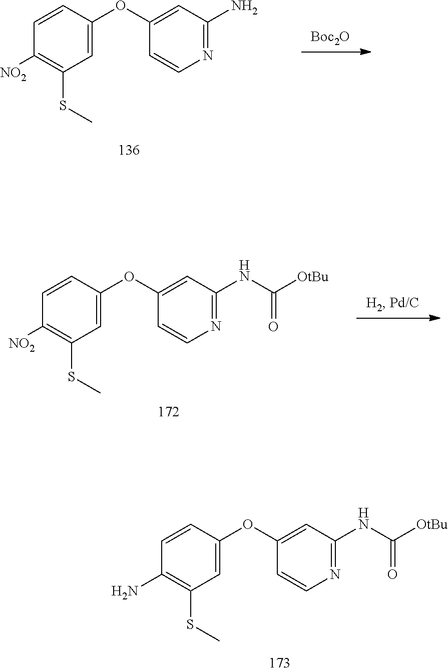 [SMILES: "Boc2O" ; CSc1cc(Oc2ccnc(N)c2)ccc1[N+](=O)[O-] ; CSc1cc(Oc2ccnc(NC(=O)OC(C)(C)C)c2)ccc1[N+](=O)[O-] ; CSc1cc(Oc2ccnc(NC(=O)OC(C)(C)C)c2)ccc1N]

### Step 1. Synthesis of Compound (172)

To a stirred suspension of 4-(3-methylsulfanyl-4-nitro-phenoxy)pyridin-2-amine (1.10 g, 3.97 mmol) in tBuOH (9 mL) was added at 0° C. Boc₂O (1.26 g, 5.78 mmol). The mixture was heated at 50° C. for 6 h. After cooling at room temperature, ethanol (9 mL) was added. The suspension was filtered off and washed with methanol (4 mL). The solid obtained was dried under vacuum leading to the expected product 172 (Yield: 78%, 1.17 g) as a yellow solid.

### Step 2. Synthesis of Compound (173)

A suspension of tert-butyl N-[4-(3-methylsulfanyl-4-nitro-phenoxy)-2-pyridyl] carbamate 172 (1.17 g, 3.12 mmol) and Pd/C (150 mg) in a mixture ethanol/dichloromethane (3:1; 200 mL) was stirred under hydrogen (4 bars). After 4 h at room temperature and work-up, the expected product 173 (Yield: 95%, 1.03 g) was recovered as a green powder.

### Synthesis of 178: 8-(4-amino-3-isopropylphenoxy)pyrido[2,3-b]pyrazin-3(4H)-one Hydrochloride

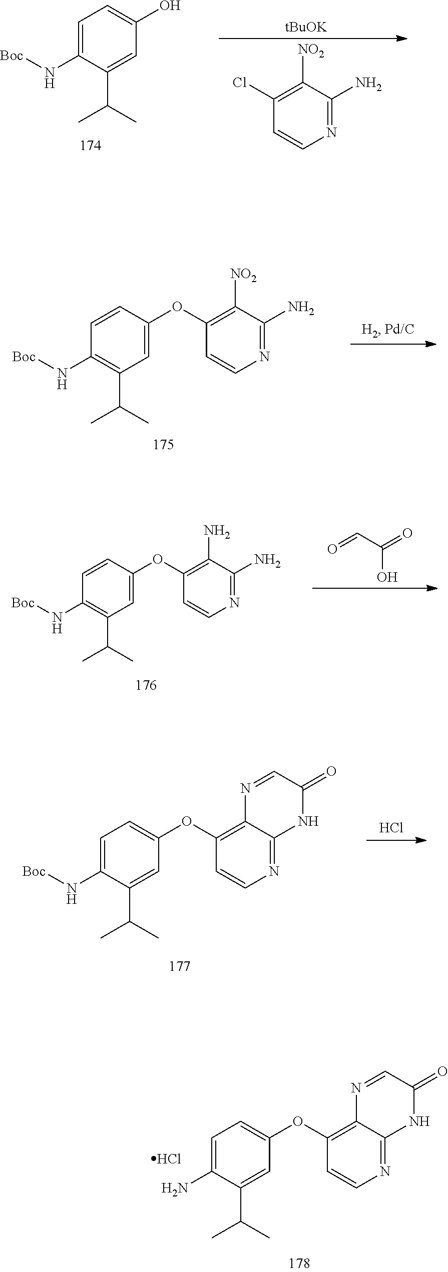

### Step 1. Synthesis of Compound (175)

Potassium tert-butoxide (1.1 g, 9.8 mmol) was added at room temperature to a solution of tert-butyl N-[4-hydroxy-2-isopropyl-phenyl]carbamate 174 (1.9 g, 7.6 mmol) in DMF (50 mL). After 15 min, 4-chloro-3-nitro-pyridin-2-amine (1.3 g, 7.6 mmol) was added. The reaction mixture was heated at 80° C. for 2 h30. After cooling at room temperature and evaporation of the solvent under vacuum, the crude obtained was diluted in AcOEt and washed with water. The suspension was filtered off (washing with ether), leading after drying to the expected product 175 (Yield: 74%, 2.1 g) as a yellow powder.

### Step 2. Synthesis of Compound (176)

Palladium on activated charcoal (around 200 mg) was added to a solution of tert-butyl N-{4-[(2-amino-3-nitro-4-pyridyl)oxy]-2-isopropyl-phenyl}carbamate 175 (2.1 g, 5.56 mmol) in ethanol (100 ml) and dichloromethane (10 ml). The reaction mixture was hydrogenated at room temperature over 1 atm of hydrogen. After 18 h at RT and work-up, the expected product 176 (Yield: 79%, 1.58 g) was recovered as a brown foam.

### Step 3. Synthesis of Compound (177)

Tert-butyl N-{4-[(2,3-diamino-4-pyridyl)oxy]-2-isopropylphenyl}carbamate 176 (1.58 g, 4.40 mmol) in methanol was added dropwise to a solution of glyoxylic acid (4.0 g, 44 mmol) in methanol (30 mL). The mixture was stirred at room temperature for 18 h. After work-up, the expected product 177 (Yield: 50%, 870 mg) was recovered as a white powder.

### Step 4. Synthesis of Compound (178)

Tert-butyl N-{4-[(3-oxo-4H-pyrido[2,3-b]pyrazin-8-yl)oxy]-2-isopropyl-phenyl}carbamate 177 (870 mg, 2.2 mmol) was treated with HCl 4N in dioxane (10 mL) and the reaction mixture was stirred at room temperature for 4 h. After concentration under vacuum, the beige powder 178 recovered (821 mg) was directly used for next step without any purification.

### Synthesis of 180: 3-(tert-butyl)-1-cyclohexyl-1H-pyrazol-5-amine

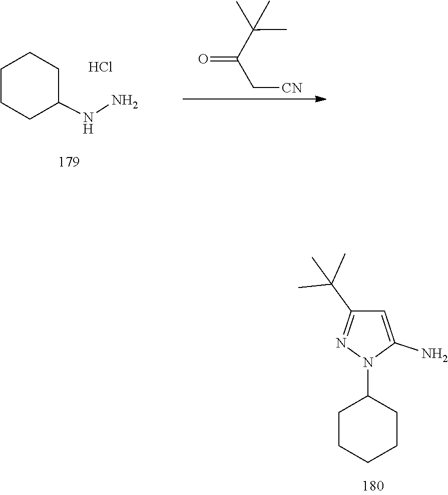

Cyclohexylhydrazine hydrochloride 179 (1.0 g, 6.64 mmol, 1.0 eq) and pivaloyl acetonitrile (831 mg, 6.64 mmol, 1.0 eq) in ethanol wad added hydrochloric acid 12 N (10.0 eq). The mixture was stirred at reflux for 18 h. After cooling down, sodium hydrogencarbonate solid was added until pH was around 7. After the extraction, the organic phases were dried over magnesium sulfate, filtered and evaporated to afford the expected compound 180 as pale brown solid (Yield: 95%, 1.39 g).

### Synthesis of 186: 8-(4-amino-2,3-difluorophenoxy)pyrido[2,3-b]pyrazin-3(4H)-one Hydrochloride

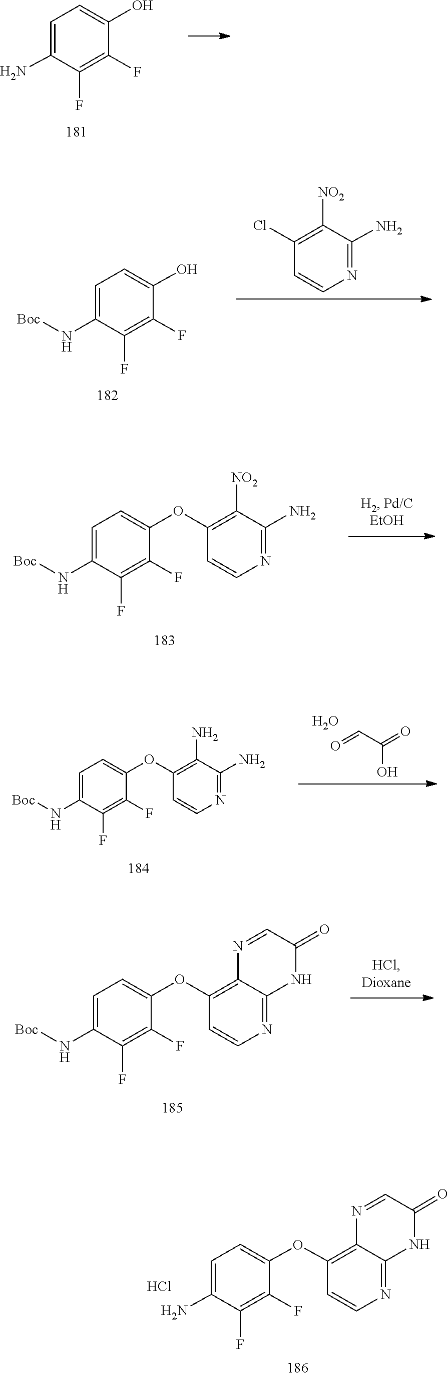

### Step 1. Synthesis of Compound (182)

4-Amino-2,3-difluoro-phenol 181 (4.9 g, 33.9 mmol, 1.0 eq) was added to a molten mixture of di-tert-butyl dicarbonate (7.4 g, 33.9 mmol, 1.0 eq) and indium (Ill) chloride (75 mg, 0.34 mmol, 0.01 eq). The reaction mixture was stirred at 35° C. for 4 hours. After cooling down, flash chromatography on silica gel was performed using cyclohexane and ethyl acetate to afford expected compound in mixture with 30% of 0 protected BOC compound (Yield: 14%, 1.68 g) as brown gum.

### Step 2. Synthesis of Compound (183)

Tert-butyl N-(2,3-difluoro-4-hydroxy-phenyl)carbamate in mixture with 30% of 0 protected BOC compound (1.68 g, 6.85 mmol, 1.0 eq) in 60 mL of DMF was added potassium tert-butylate (999 mg, 8.9 mmol, 1.3 eq). After stirring 30 min at room temperature, 4-chloro-2-amino-3-nitropyridine (1.19 g, 6.85 mmol, 1.0 eq) was added and the mixture was heated at 80° C. for 3 h. The crude was purified by flash chromatography on silica gel was performed using cyclohexane and ethyl acetate to afford expected compound 183 (Yield: 68%, 2.2 g) as yellow solid.

### Step 3. Synthesis of Compound (184)

Palladium on activated charcoal (around 350 mg) was added to a solution of tert-butyl N-[4-[(2-amino-3-nitro-4-pyridyl)oxy]-2,3-difluoro-phenyl]carbamate 183 (2.2 g, 5.8 mmol, 1.0 eq) in ethanol (280 mL). The reaction mixture was hydrogenated at room temperature over 1 atmosphere of hydrogen for 20 h. The reaction mixture was filtrated through a short pad of celite and rinsed with methanol. The filtrate was evaporated to dryness to afford the expected compound 184 as brown foam (Yield: 98%, 2.0 g).

### Step 4. Synthesis of Compound (185)

Tert-butyl N-[4-[(2,3-diamino-4-pyridyl)oxy]-2,3-difluoro-phenyl]carbamate 184 (2.0 g, 5.67 mmol, 1.0 eq) in methanol was added dropwise to a solution of glyoxylic acid monohydrate (5.2 g, 56.7 mmol, 10.0 eq) in methanol (40 mL). The mixture was stirred at room temperature for 18 h. After work-up and filtration, the expected product 185 was obtained as pale grey solid (Yield: 47%, 1.04 g).

### Step 5. Synthesis of Compound (186)

Tert-butyl-N-[2,3-difluoro-4-[(3-oxo-4H-pyrido[2,3-b]pyrazin-8-yl)oxy]phenyl]carbamate 185 (1.04 g, 2.6 mmol, 1.0 eq) was treated with HCl 4N in dioxane (21 mL) and the reaction mixture was stirred at room temperature for 4 h. The precipitate was filtered, washed with diethyl ether and dried under vacuo to afford expected compound as hydrochloride salt 186 as pale grey powder (1.0 g).

### Synthesis of 188: 2-(methylthio)-4-(pyrido[2,3-b]pyrazin-8-yloxy)aniline Hydrochloride

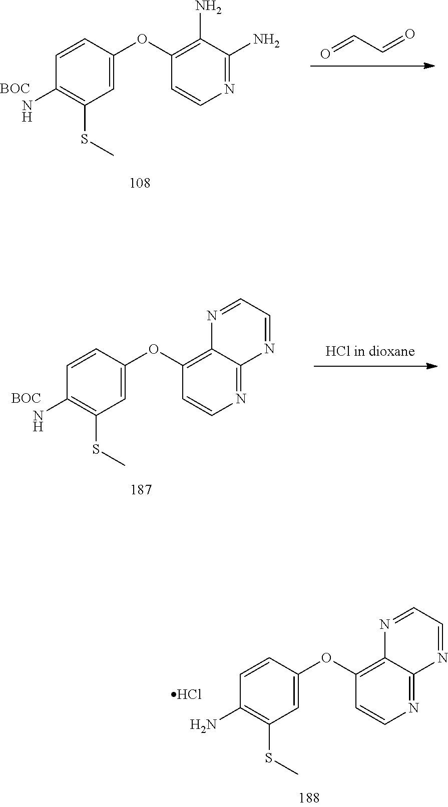

### Step 1. Synthesis of Compound (187)

A solution of tert-butyl N-{4-[(2,3-diamino-4-pyridyl)oxy]-2-methylsulfanyl-phenyl} carbamate 108 (500 mg, 1.38 mmol) and glyoxal (40% in water, 0.26 mL, 1.79 mmol) in THF (10 mL) was stirred at room temperature for 18 h. After evaporation of the solvent, the crude obtained was purified by flash chromatography (40 g, SiO₂, cyclohexane/AcOEt 100:0→0:100, 10 CV and AcOEt/CH₃OH 100:0→90:10, 5 CV) leading to the expected product 187 (Yield: 87%, 530 mg) as a beige solid.

### Step 2. Synthesis of Compound (188)

Tert-butyl N-(2-methylsulfanyl-4-pyrido[2,3-b]pyrazin-8-yloxy-phenyl)carbamate 187 (530 mg, 1.38 mmol) was treated with 4N HCl in dioxane (15 mL) and the reaction mixture was stirred at room temperature for 3 h. The suspension obtained was filtered off, washed with ether, and dried under vacuum. The dark blue solid 188 recovered (529 mg, quantitative yield) was directly used for next step without any purification.

### Synthesis of 195: 8-(4-amino-2,5-difluorophenoxy)pyrido[2,3-b]pyrazin-3(4H)-one Hydrochloride

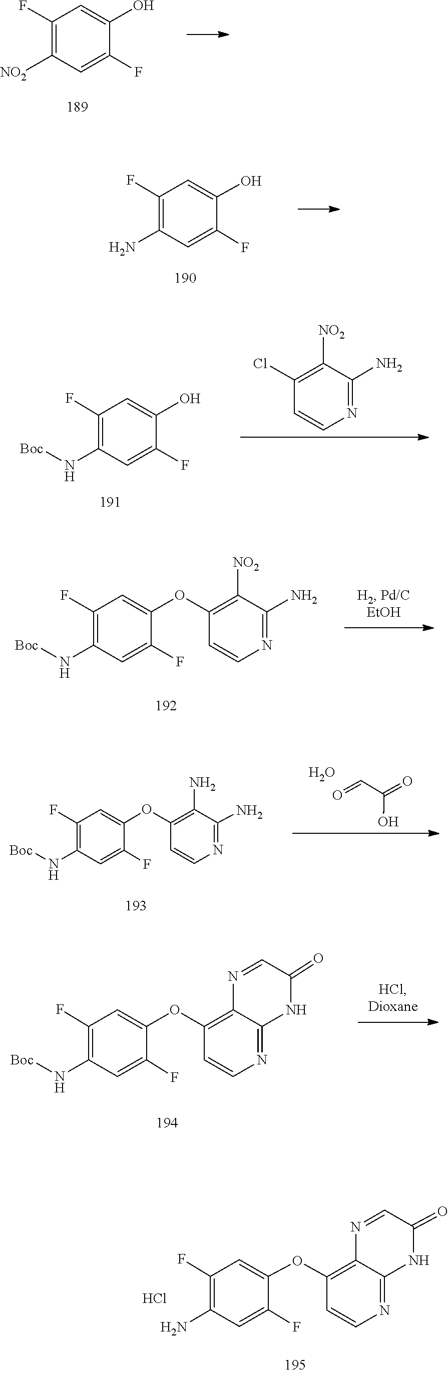

### Step 1. Synthesis of Compound (190)

Platinum oxide (around 30 mg) was added to a solution of 2,5-difluoro-4-nitrophenol 189 (3.73 g, 21.30 mmol, 1.0 eq) in methanol (150 mL). The reaction mixture was hydrogenated at room temperature over 4 atmospheres of hydrogen for 4 h. The reaction mixture was filtrated through a short pad of celite and rinsed with methanol. The filtrate was evaporated to dryness to afford the expected compound 190 as black solid (Yield: 96%, 2.96 g). The compound, light and air sensitive, was used directly in the next step without further purification.

### Step 2. Synthesis of Compound (191)

4-Amino-2,5-difluoro-phenol 190 (1.7 g, 11.58 mmol, 1.0 eq) was added to a molten mixture of di-tert-butyl dicarbonate (2.5 g, 11.58 mmol, 1.0 eq) and indium (III) chloride (26 mg, 0.11 mmol, 0.01 eq). The reaction mixture was stirred at 35° C. for 4 hours. After cooling down, flash chromatography on silica gel was performed using cyclohexane and ethyl acetate to afford expected compound 191 (Yield: 53%, 1.5 g, 53%) as yellow oil.

### Step 3. Synthesis of Compound (192)

Tert-butyl N-(2,5-difluoro-4-hydroxy-phenyl)carbamate 191 (1.5 g, 6.1 mmol, 1.0 eq) in 60 mL of DMF was added potassium tert-butylate (892 mg, 7.9 mmol, 1.3 eq). After stirring 30 min at room temperature, 4-chloro-2-amino-3-nitropyridine (1.06 g, 6.1 mmol, 1.0 eq) was added and the mixture was heated at 80° C. for 3 h. The crude was purified by flash chromatography on silica gel using cyclohexane and ethyl acetate to afford expected compound 192 (Yield: 78%, 2.3 g) as yellow solid.

### Step 4. Synthesis of Compound (193)

Palladium on activated charcoal (around 350 mg) was added to a solution of tert-butyl N-[4-[(2-amino-3-nitro-4-pyridyl)oxy]-2,5-difluoro-phenyl]carbamate 192 (2.3 g, 6.01 mmol, 1.0 eq) ethanol (150 ml). The reaction mixture was hydrogenated at room temperature over 1 atmosphere of hydrogen for 20 h. The reaction mixture was filtrated through a short pad of celite and rinsed with methanol. The filtrate was evaporated to dryness to afford the expected compound 193 as dark brown foam (Yield: 99%, 2.1 g).

### Step 5. Synthesis of Compound (194)

Tert-butyl N-[4-[(2,3-diamino-4-pyridyl)oxy]-2,5-difluoro-phenyl]carbamate 193 (2.1 g, 5.96 mmol, 1.0 eq)) in methanol was added dropwise to a solution of glyoxylic acid monohydrate (5.5 g, 59.6 mmol, 10.0 eq) in methanol (42 mL). The mixture was stirred at room temperature for 18 h. After work-up and filtration, the expected product 194 was obtained as beige solid after filtration (Yield: 56%, 1.3 g).

### Step 6. Synthesis of Compound (195)

Tert-butyl-N-[2,5-difluoro-4-[(3-oxo-4H-pyrido[2,3-b]pyrazin-8-yl)oxy]phenyl]carbamate 194 (1.3 g, 3.33 mmol, 1.0 eq) was treated with 4N HCl in dioxane (27 mL) and the reaction mixture was stirred at room temperature for 3 h. The suspension obtained was filtered off, washed with ether, and dried under vacuum to afford expected compound as hydrochloride salt 195 as beige powder (1.2 g, quantitative yield).

### Synthesis of 199

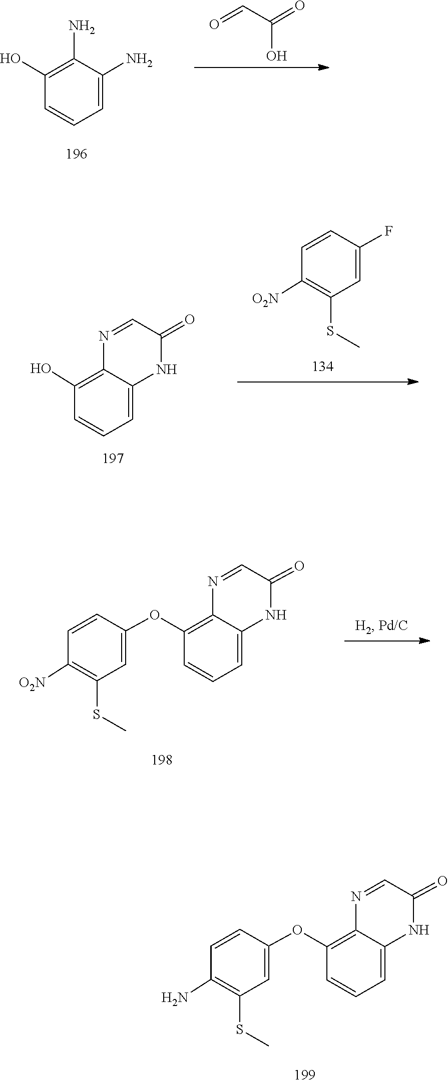 [SMILES: Nc1cccc(O)c1N ; O=CC(=O)O ; O=c1cnc2c(O)cccc2[nH]1 ; CSc1cc(F)ccc1[N+](=O)[O-] ; CSc1cc(Oc2cccc3[nH]c(=O)cnc23)ccc1[N+](=O)[O-] ; CSc1cc(Oc2cccc3[nH]c(=O)cnc23)ccc1N]

### Step 1. Synthesis of Compound (197)

2,3-diaminophenol 196 (3.0 g, 24.2 mmol) in methanol was added dropwise to a solution of glyoxylic acid (22 g, 242 mmol) in methanol (20 mL). The mixture was stirred at room temperature for 18 h. After work-up and filtration, the expected product (Yield: 61%, 2.4 g) was recovered as a mixture of regioisomers (brown powder).

### Step 2. Synthesis of Compound (198)

In a sealed tube, DBU (3.7 mL, 24.8 mmol), 5-hydroxy-1H-quinoxalin-2-one 197 (2.9 g, 17.9 mmol) in dry acetonitrile (0.1M). The mixture was stirred at room temperature for 30 min before addition of 4-fluoro-2-methylsulfanyl-1-nitro-benzene 134 (2.6 g, 13.8 mmol). The reaction mixture was stirred at 80° C. for 5 h. After cooling to room temperature the suspension was filtered off and the powder was purified by flash chromatography (120 g, SiO₂, cyclohexane/AcOEt 100:0→0:100, 10 CV and AcOEt/CH₃OH 100:0→90:10, 5 CV) leading to the expected product (Yield: 7%, 322 mg) as a yellow solid.

### Step 3. Synthesis of Compound (199)

A suspension of 5-(3-methylsulfanyl-4-nitro-phenoxy)-1H-quinoxalin-2-one 198 (320 mg, 0.99 mmol) and Pd/C (50 mg) in a mixture ethanol/dichloromethane (5:2; 70 mL) was stirred under hydrogen (4 bars). After 5 h at room temperature and work-up, the expected product 199 (308 mg, quantitative yield) was recovered as an orange foam.

### Synthesis of 203: 4-(4-amino-3-(methylthio)phenoxy)pteridin-7(8H)-one Hydrochloride

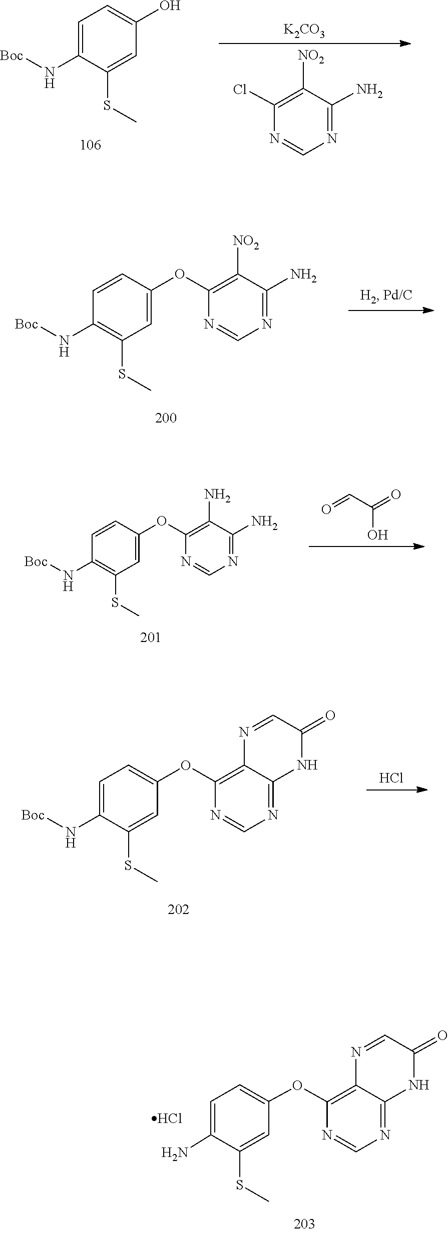

### Step 1. Synthesis of Compound (200)

Potassium carbonate (890 mg, 6.5 mmol) was added at room temperature to a solution of tert-butyl N-(4-hydroxy-2-methylsulfanyl-phenyl)carbamate 106 (1.1 g, 4.3 mmol) in DMF (25 mL). After 15 min, 6-chloro-5-nitro-pyrimidin-4-amine (1.9 g, 10.8 mmol) was added. The reaction mixture was heated at 90° C. for 18 h. After cooling at room temperature and evaporation of the solvent under vacuum, the crude obtained was diluted in AcOEt and washed with water. The organic layer was dried over MgSO₄, filtered, and concentrated in vacuo. The crude obtained was purified by flash chromatography (40 g, SiO₂, cyclohexane/AcOEt 100:0→60:40, 10 CV), leading to the expected product 200 (Yield: 30%, 498 mg) as a yellow powder.

### Step 2. Synthesis of Compound (201)

Palladium on activated charcoal (around 100 mg) was added to a solution of tert-butyl N-[4-(6-amino-5-nitro-pyrimidin-4-yl)oxy-2-methylsulfanyl-phenyl]carbamate 200 (770 mg, 1.97 mmol) in ethanol (20 mL). The reaction mixture was hydrogenated at room temperature with 5 bars of hydrogen for 20 h. After work-up, the expected product 201 (Yield: 89%, 638 mg) was recovered.

### Step 3. Synthesis of Compound (202)

Tert-butyl N-[4-(5,6-diaminopyrimidin-4-yl)oxy-2-methylsulfanyl-phenyl]carbamate 201 (638 mg, 1.75 mmol) in methanol was added dropwise to a solution of glyoxylic acid (1.6 g, 17.5 mmol) in methanol (23 mL). The mixture was stirred at room temperature for 18 h. After work-up, the crude was purified by flash chromatography (40 g, SiO₂, CH₂Cl₂/AcOEt 100:0→80:20, 10 CV) leading to the expected product 202 (Yield: 16%, 112 mg).

### Step 4. Synthesis of Compound (203)

Tert-butyl N-{2-methylsulfanyl-4-[(7-oxo-8H-pteridin-4-yl)oxy]phenyl}carbamate 202 (112 mg, 0.28 mmol) was treated with HCl 4N in dioxane (10 mL) and the reaction mixture was stirred at room temperature for 4 h. The suspension was filtered off, washed with ether, and dried under vacuum leading to a beige powder 203 recovered (Yield: 86%, 81 mg) which was directly used for next step without any purification.

### Synthesis of 206: 2,2,2-trichloroethyl (3-(tert-butyl)-1-(4-cyanophenyl)-1H-pyrazol-5-yl)carbamate

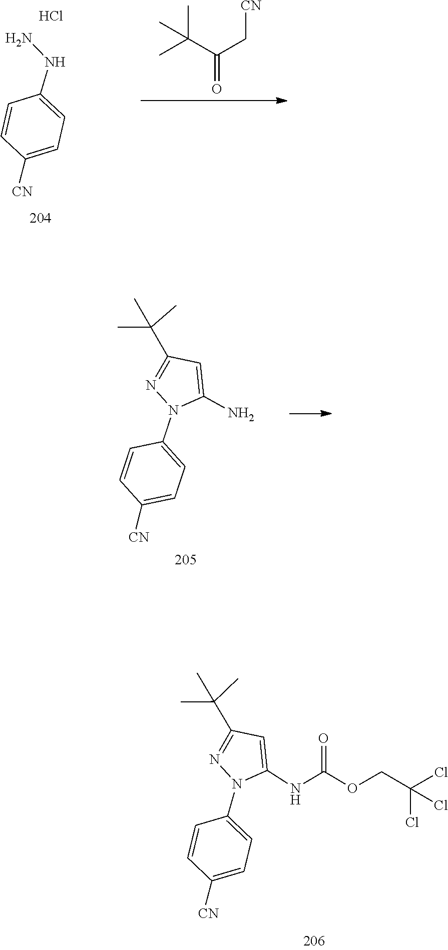

### Step 1. Synthesis of Compound (205)

4-hydrazinobenzonitrile hydrochloride 204 (1.0 g, 5.9 mmol, 1.0 eq) and pivaloyl acetonitrile (738 mg, 5.9 mmol, 1.0 eq) in ethanol wad added hydrochloric acid 12 N (10.0 eq). The mixture was stirred at reflux for 18 h. The solution was basified with a saturated solution of hydrogencarbonate and extracted with ethyl acetate. The organic phases were dried over magnesium sulfate, filtered and evaporated to afford expected compound 205 as orange solid (Yield: 99%, 1.4 mg).

### Step 2. Synthesis of Compound (206)

At 0° C., 4-(5-amino-3-tert-butyl-pyrazol-1-yl)benzonitrile 205 (400 mg, 1.66 mmol, 1.0 eq) was added dropwise dry pyridine (1.1 eq) followed by 2,2,2-trichloroethylchloroformate (0.25 mL, 1.83 mmol, 1.1 eq). The reaction mixture was stirred at room temperature for 18 h. Water was added, and the reaction mixture was extracted with ethyl acetate. The organic layers were combined, dried over magnesium sulfate, filtered and evaporated. Compound 206 was obtained as yellow gum (500 mg). The compound was used without purification in the next step.

### Synthesis of 209: 2,2,2-trichloroethyl (3-(tert-butyl)-1-(3,4-dimethylphenyl)-1H-pyrazol-5-yl)carbamate

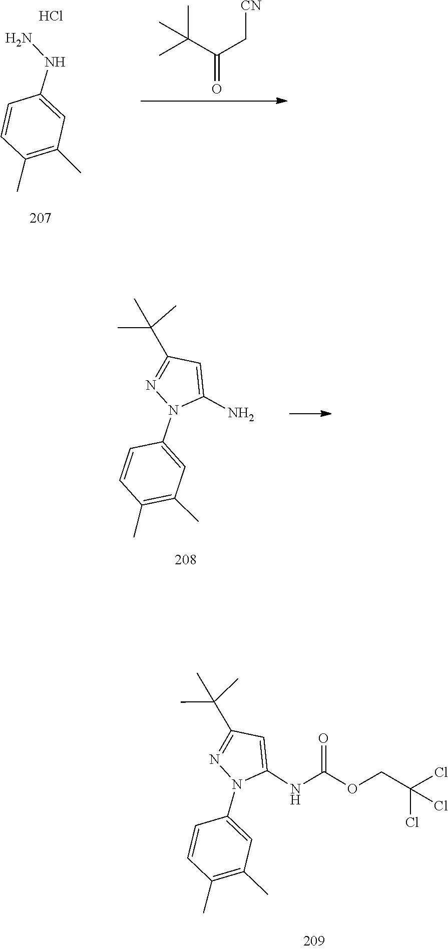

### Step 1. Synthesis of Compound (208)

(3,4-dimethylphenyl)hydrazine hydrochloride 207 (2.0 g, 11.6 mmol, 1.0 eq) and pivaloyl acetonitrile (2.3 g, 18.5 mmol, 1.0 eq) in ethanol wad added hydrochloric acid 12 N (10.0 eq). The mixture was stirred at reflux for 18 h. The solution was basified with a saturated solution of hydrogencarbonate and extracted with ethyl acetate. The organic phases were dried over magnesium sulfate, filtered and evaporated. The residue was purified by flash chromatography over silica gel using cyclohexane and ethyl acetate to afford expected compound 208 as orange solid (2.9 g, quantitative yield).

### Step 2. Synthesis of Compound (209)

At 0° C., 5-tert-butyl-2-(3,4-dimethylphenyl)pyrazol-3-amine 208 (1.0 g, 4.11 mmol, 1.0 eq) was added dropwise dry pyridine (1.1 eq) followed by 2,2,2-trichloroethylchloroformate (0.62 mL, 4.52 mmol, 1.1 eq). The reaction mixture was stirred at room temperature for 18 h. Water was added, and the reaction mixture was extracted with ethyl acetate. The organic layers were combined, dried over magnesium sulfate, filtered and evaporated. The compound 209 was obtained as beige solid (1.7 g, quantitative yield) and was used without purification in the next step.

### Synthesis of 142: 2,2,2-trichloroethyl (3-(tert-butyl)-1-(4-(2-(piperidin-1-yl)ethoxy)phenyl)-1H-pyrazol-5-yl)carbamate

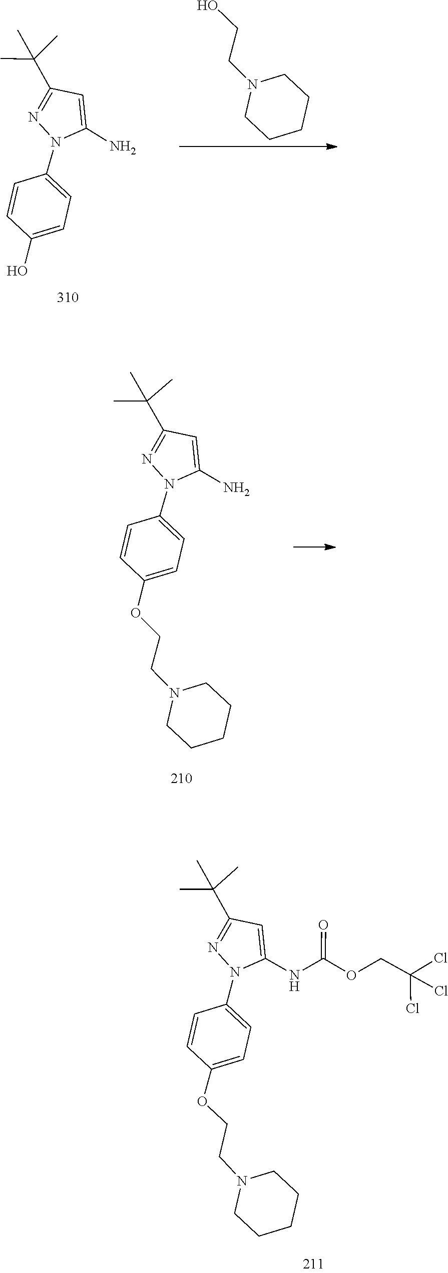 [SMILES: CC(C)(C)c1cc(N)n(-c2ccc(O)cc2)n1 ; OCCN1CCCCC1 ; CC(C)(C)c1cc(N)n(-c2ccc(OCCN3CCCCC3)cc2)n1 ; CC(C)(C)c1cc(NC(=O)OCC(Cl)(Cl)Cl)n(-c2ccc(OCCN3CCCCC3)cc2)n1]

### Step 1. Synthesis of Compound (210)

Diisopropyl azodicarboxylate (0.53 mL, 2.7 mmol, 1.9 eq) was added at room temperature to a solution of 4-(5-amino-3-(tert-butyl)-1H-pyrazol-1-yl)phenol 310 (330 mg, 1.42 mmol, 1.0 eq), 2-(1-piperidyl)ethanol (0.22 mL, 1.7 mmol, 1.2 eq) and triphenylphosphine (708 mg, 2.7 mmol, 1.9 eq) in 10 mL of THF. The reaction mixture was stirred at room temperature for 20 h. A 10% solution of citric acid was added, and the mixture was extracted with ether. The aqueous phase was carefully basified with potassium carbonate and extracted with ethyl acetate. The organic phases were dried over magnesium sulfate, filtered and evaporated. The residue was purified by flash chromatography over silica gel using ethyl acetate to afford the expected compound as yellow oil (Yield: 53%, 260 mg).

### Step 2. Synthesis of Compound (211)

At 0° C., 5-tert-butyl-2-[4-[2-(1-piperidyl)ethoxy]phenyl]pyrazol-3-amine 210 (260 mg, 0.76 mmol, 1.0 eq) was added dropwise dry pyridine (1.1 eq) followed by 2,2,2-trichloroethylchloroformate (0.11 mL, 0.83 mmol, 1.1 eq). The reaction mixture was stirred at room temperature for 18 h. Water was added, and the reaction mixture was extracted with ethyl acetate. The organic layers were combined, dried over magnesium sulfate, filtered and evaporated. The compound 211 was obtained as orange oil (614 mg, quantitative yield) and was used without purification in the next step.

### Synthesis of 213: 2,2,2-trichloroethyl (3-(tert-butyl)-1-(4-(2-morpholinoethoxy)phenyl)-1H-pyrazol-5-yl)carbamate

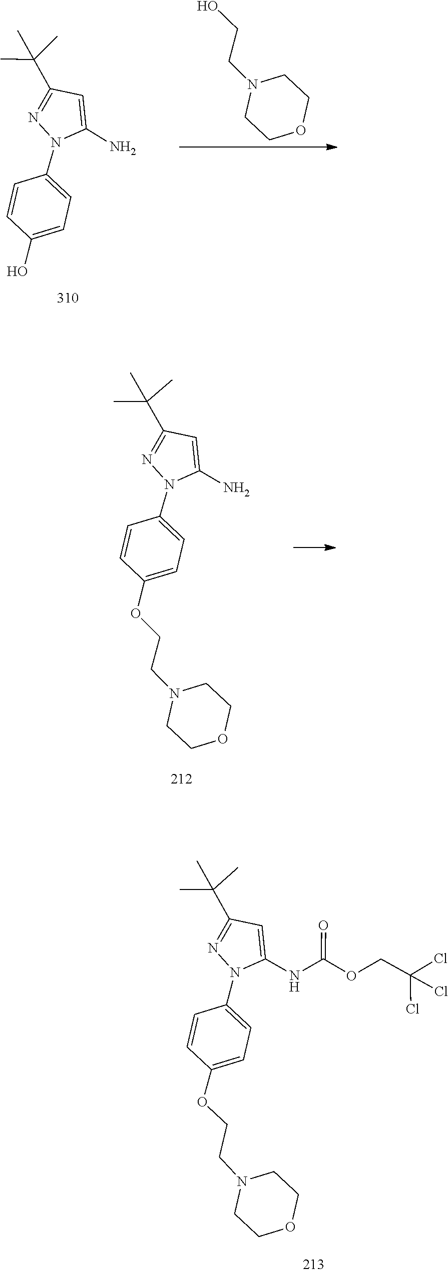 [SMILES: CC(C)(C)c1cc(N)n(-c2ccc(O)cc2)n1 ; OCCN1CCOCC1 ; CC(C)(C)c1cc(N)n(-c2ccc(OCCN3CCOCC3)cc2)n1 ; CC(C)(C)c1cc(NC(=O)OCC(Cl)(Cl)Cl)n(-c2ccc(OCCN3CCOCC3)cc2)n1]

### Step 1. Synthesis of Compound (212)

Diisopropyl azodicarboxylate (0.56 mL, 2.8 mmol, 2.0 eq) was added at room temperature to a solution of 4-(5-amino-3-(tert-butyl)-1H-pyrazol-1-yl)phenol 310 (330 mg, 1.42 mmol, 1.0 eq), 2-morpholino-ethanol (0.21 mL, 1.7 mmol, 1.2 eq) and triphenylphosphine (745 mg, 2.8 mmol, 2.0 eq) in 10 mL of THF. The reaction mixture was stirred at room temperature for 20 h. A 10% solution of citric acid was added, and the mixture was extracted with ether. The aqueous phase was carefully basified with potassium carbonate and extracted with ethyl acetate. The organic phases were dried over magnesium sulfate, filtered and evaporated. The residue was purified by flash chromatography over silica gel using ethyl acetate first and then dichloromethane and methanol to afford the expected compound 212 as orange oil (Yield: 61%, 300 mg).

### Step 2. Synthesis of Compound (213)

At 0° C., 5-tert-butyl-2-[4-[2-morpholino-ethoxy]phenyl]pyrazol-3-amine 212 (300 mg, 0.87 mmol, 1.0 eq) was added dropwise dry pyridine (1.1 eq) followed by 2,2,2-trichloroethylchloroformate (0.13 mL, 0.96 mmol, 1.1 eq). The reaction mixture was stirred at room temperature for 18 h. Water was added, and the reaction mixture was extracted with ethyl acetate. The organic layers were combined, dried over magnesium sulfate, filtered and evaporated. The compound 213 was obtained as orange oil (606 mg, quantitative yield) and was used without purification in the next step.

### Synthesis of 216: 2,2,2-trichloroethyl (3-(tert-butyl)-1-(3-chloro-4-methylphenyl)-1H-pyrazol-5-yl)carbamate

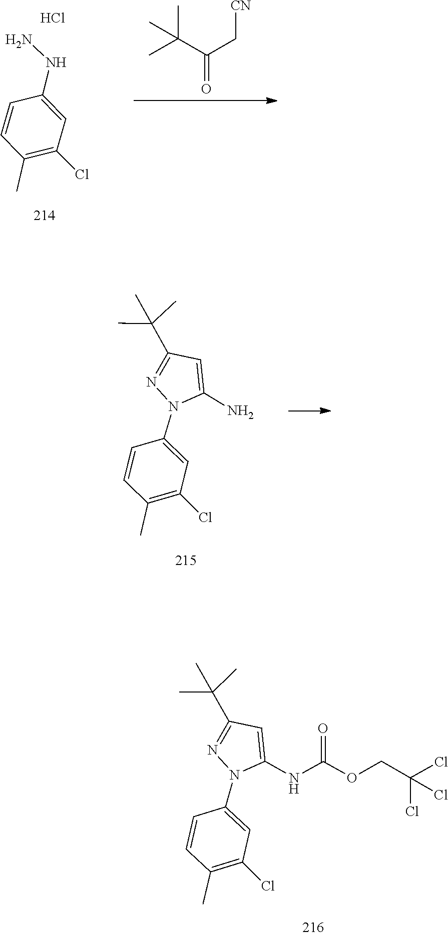

### Step 1. Synthesis of Compound (215)

(3-chloro-4-methyl-phenyl)hydrazine hydrochloride 214 (500 mg, 2.6 mmol, 1.0 eq) and pivaloyl acetonitrile (519 mg, 4.1 mmol, 1.6 eq)) in ethanol wad added hydrochloric acid 12 N (10.0 eq). The mixture was stirred at reflux for 18 h. The solution was basified with a saturated solution of hydrogencarbonate and extracted with ethyl acetate. The organic phases were dried over magnesium sulfate, filtered and evaporated. The residue was purified by flash chromatography over silica gel using cyclohexane and ethyl acetate to afford expected compound 215 as yellow oil (740 mg, quantitative yield).

### Step 2. Synthesis of Compound (216)

At 0° C., 5-tert-butyl-2-(3-chloro-4-methyl-phenyl)pyrazol-3-amine 215 (740 mg, 2.81 mmol, 1.0 eq) was added dropwise dry pyridine (1.1 eq) followed by 2,2,2-trichloroethylchloroformate (0.43 mL, 3.09 mmol, 1.1 eq). The reaction mixture was stirred at room temperature for 18 h. Water was added, and the reaction mixture was extracted with ethyl acetate. The organic layers were combined, dried over magnesium sulfate, filtered and evaporated. The compound 216 was obtained as pale yellow solid (1.73 g, quant. yield) and was used without purification in the next step.

### Synthesis of 219: 2,2,2-trichloroethyl (3-(tert-butyl)-1-(3-chloro-4-fluorophenyl)-1H-pyrazol-5-yl)carbamate

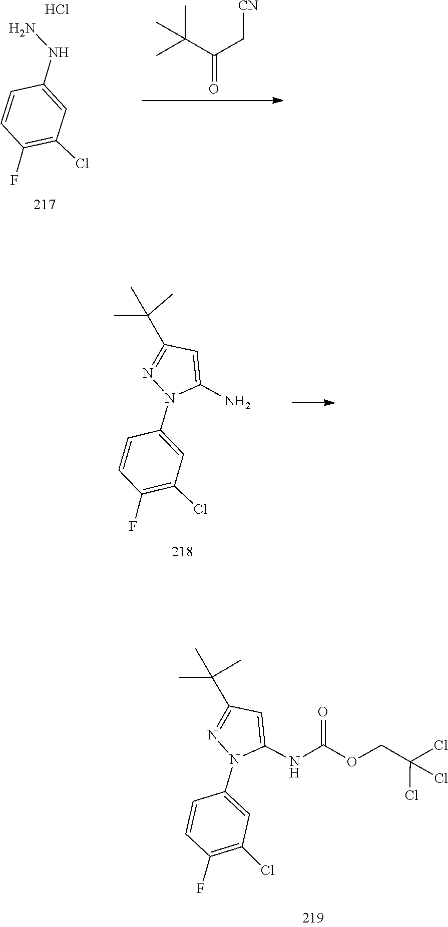

### Step 1. Synthesis of Compound (218)

(3-chloro-4-fluoro-phenyl)hydrazine hydrochloride 217 (2.0 g, 10.15 mmol, 1.0 eq) and pivaloyl acetonitrile (2.0 g, 16.29 mmol, 1.6 eq) in ethanol wad added hydrochloric acid 12N (10.0 eq). The mixture was stirred at reflux for 18 h. The solution was basified with a saturated solution of hydrogencarbonate and extracted with ethyl acetate. The organic phases were dried over magnesium sulfate, filtered and evaporated. The residue was purified by flash chromatography over silica gel using cyclohexane and ethyl acetate to afford expected compound 218 as crystallized orange oil (2.7 g, quantitative yield).

### Step 2. Synthesis of Compound (219)

At 0° C., 5-tert-butyl-2-(3-chloro-4-fluoro-phenyl)pyrazol-3-amine 218 (1.0 g, 3.74 mmol, 1.0 eq) was added dropwise dry pyridine (1.1 eq) followed by 2,2,2-trichloroethylchloroformate (0.57 mL, 4.11 mmol, 1.1 eq). The reaction mixture was stirred at room temperature for 18 h. Water was added, and the reaction mixture was extracted with ethyl acetate. The organic layers were combined, dried over magnesium sulfate, filtered and evaporated. Compound 219 was obtained as yellow oil (1.9 g, quantitative yield) and was used without purification in the next step.

### Synthesis of 221: 4-((1H-indazol-4-yl)oxy)-2-(methylthio)aniline

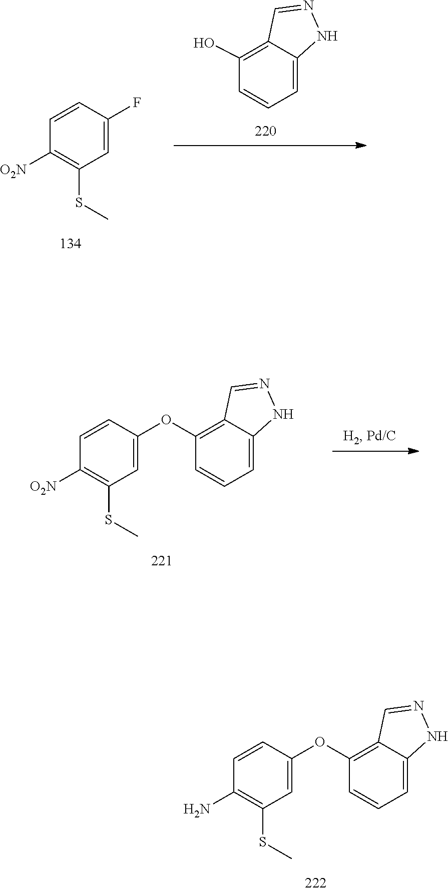 [SMILES: CSc1cc(F)ccc1[N+](=O)[O-] ; Oc1cccc2[nH]ncc12 ; CSc1cc(Oc2cccc3[nH]ncc23)ccc1[N+](=O)[O-] ; CSc1cc(Oc2cccc3[nH]ncc23)ccc1N]

### Step 1. Synthesis of Compound (221)

In a sealed tube, DBU (0.28 mL, 1.90 mmol), 1H-indazol-4-ol 220 (186 mg, 1.40 mmol) in dry acetonitrile (0.1M). The mixture was stirred at room temperature for 30 min before addition of 4-fluoro-2-methylsulfanyl-1-nitro-benzene 134 (200 mg, 1.06 mmol). The reaction mixture was stirred at 80° C. for 5 h. After cooling to room temperature the suspension was filtered off and washed with Et₂O leading to the expected product 221 (Yield: 28%, 90 mg) as a yellow oil.

### Step 2. Synthesis of Compound (222)

A suspension of 4-(3-methylsulfanyl-4-nitro-phenoxy)-1H-indazole 221 (90 mg, 0.30 mmol) and Pd/C (10 mg) in ethanol (15 mL) and dichloromethane (3 mL) was stirred under hydrogen (1 atm). After 18 h at room temperature and work-up, the expected product 222 (80 mg, quantitative yield) was recovered as a purple oil.

### Synthesis of 224: 4-(4-amino-3-(methylthio)phenoxy)-N-phenylpicolinamide

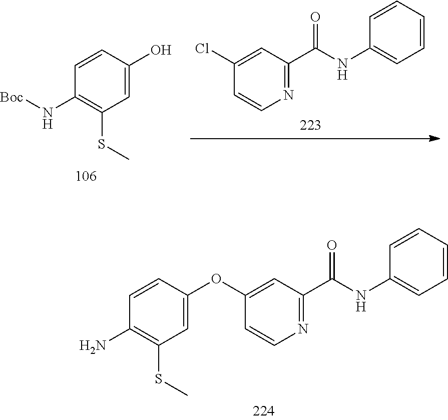 [SMILES: CSc1cc(O)ccc1NC(=O)OC(C)(C)C ; O=C(Nc1ccccc1)c1cc(Cl)ccn1 ; CSc1cc(Oc2ccnc(C(=O)Nc3ccccc3)c2)ccc1N]

In a sealed tube, tBuOK (262 mg, 2.34 mmol) followed by 4-chloro-N-phenyl-pyridine-2-carboxamide 223 (363 mg, 1.56 mmol) were added at room temperature to a solution of tert-butyl N-(4-hydroxy-2-methylsulfanyl-phenyl) carbamate 106 (242 mg, 1.56 mmol) in dry DMA (3 mL). The reaction mixture was stirred at 100° C. for 18 h. After cooling to room temperature, water (50 mL), aq. sat. NH₄Cl (20 mL) and AcOEt (50 mL) were added. The aqueous layer was extracted with AcOEt (2×30 mL). The combined organic fractions were dried over MgSO₄, filtered and concentrated in vacuo. The crude obtained was purified by flash chromatography (40 g, SiO₂, cyclohexane/AcOEt 100:0→50:50, 10 CV) leading to the expected product 224 (Yield: 69%, 380 mg) as an orange gum.

### Synthesis of 231: 8-(4-amino-2-(trifluoromethoxy)phenoxy)pyrido[2,3-b]pyrazin-3(4H)-one hydrochloride

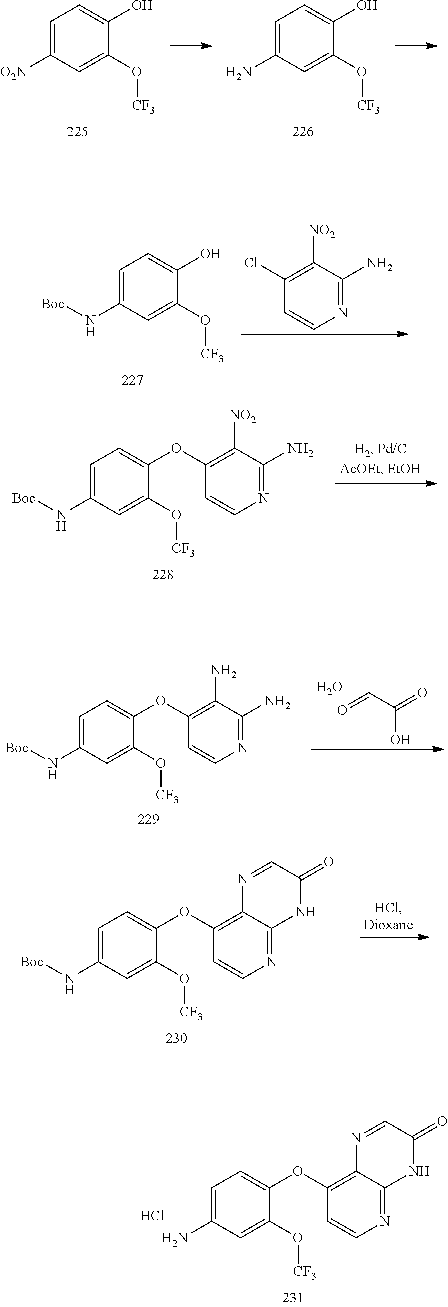

### Step 1. Synthesis of Compound (226)

Palladium on activated charcoal (around 100 mg) was added to a solution of 4-nitro-2-(trifluoromethoxy)phenol 225 (1.0 g, 4.48 mmol, 1.0 eq) in ethanol (50 mL). The reaction mixture was hydrogenated at room temperature over 1 atmosphere of hydrogen for 4 h30. The reaction mixture was filtrated through a short pad of celite and rinsed with methanol. The filtrate was evaporated to dryness to afford the expected compound 226 (970 mg, quantitative yield).

### Step 2. Synthesis of Compound (227)

4-amino-2-(trifluoromethoxy)phenol 226 (960 mg, 4.97 mmol, 1.0 eq) was added to a molten mixture of di-tert-butyl dicarbonate (1.1 g, 4.97 mmol, 1.0 eq) and indium (III) chloride (11 mg, 0.05 mmol, 0.01 eq). The reaction mixture was stirred at 35° C. for 4 hours. After cooling down, flash chromatography on silica gel was performed using cyclohexane and ethyl acetate to afford expected compound 227 (Yield: 94%, 1.37 g).

### Step 3. Synthesis of Compound (228)

Tert-butyl N-[4-hydroxy-3-(trifluoromethoxy)phenyl]carbamate 227 (1.4 g, 4.8 mmol, 1.0 eq) in 50 mL of DMF was added potassium tert-butylate (695 mg, 6.2 mmol, 1.3 eq). After stirring 30 min at room temperature, 4-chloro-2-amino-3-nitropyridine (833 mg, 4.8 mmol, 1.0 eq) was added and the mixture was heated at 80° C. for 3 h. The crude was purified by flash chromatography on silica gel was performed using cyclohexane and ethyl acetate to afford expected compound 228 (Yield: 77%, 1.6 g) as yellow solid.

### Step 4. Synthesis of Compound (229)

Palladium on activated charcoal (around 300 mg) was added to a solution of tert-butyl N-[4-[(2-amino-3-nitro-4-pyridyl)oxy]-3-(trifluoromethoxy)phenyl]carbamate 228 (1.6 g, 3.8 mmol, 1.0 eq) in ethanol (150 mL). The reaction mixture was hydrogenated at room temperature over 1 atmosphere of hydrogen for 20 h. The reaction mixture was filtrated through a short pad of celite and rinsed with methanol. The filtrate was evaporated to dryness to afford the expected compound 229 (1.5 g, quantitative yield).

### Step 5. Synthesis of Compound (230)

Tert-butyl N-[4-[(2,3-diamino-4-pyridyl)oxy]-3-(trifluoromethoxy)phenyl]carbamate 229 (1.5 g, 3.76 mmol, 1.0 eq) in methanol was added dropwise to a solution of glyoxylic acid monohydrate (3.5 g, 37.6 mmol, 10.0 eq) in methanol (54 mL). The mixture was stirred at room temperature for 18 h. After work-up and filtration, the expected compound 230 was obtained as beige solid after filtration (Yield: 53%, 874 mg).

### Step 6. Synthesis of Compound (231)

Tert-butyl-N-[4-[(3-oxo-4H-pyrido[2,3-b]pyrazin-8-yl)oxy]-3-(trifluoromethoxy)phenyl]carbamate 230 (874 mg, 1.99 mmol, 1.0 eq) was treated with 4N HCl in dioxane (16 mL) and the reaction mixture was stirred at room temperature for 4 h. After trituration in diethyl ether, the precipitate was filtered, washed with diethyl ether and dried under vacuo to afford expected compound 231 as grey powder (796 mg, quantitative yield).

### Synthesis of 238: 8-(3-amino-4-fluorophenoxy)pyrido[2,3-b]pyrazin-3(4H)-one Hydrochloride

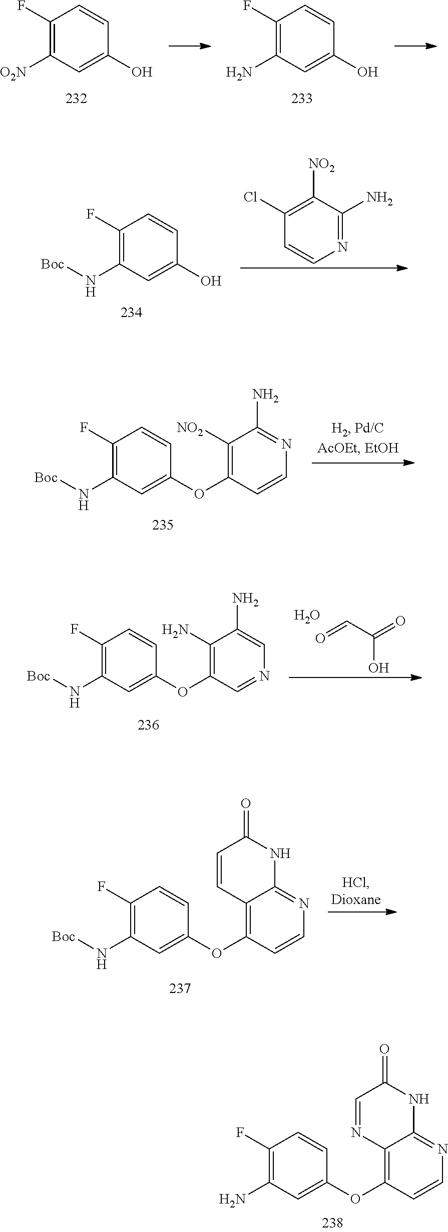

### Step 1. Synthesis of Compound (233)

Palladium on activated charcoal (around 300 mg) was added to a solution of 4-fluoro-3-nitro-phenol 232 (3.0 g, 19.0 mmol, 1.0 eq) in ethanol (100 mL). The reaction mixture was hydrogenated at room temperature over 1 atmosphere of hydrogen for 18 h. The reaction mixture was filtrated through a short pad of celite and rinsed with ethanol. The filtrate was evaporated to dryness to afford the expected compound 233 as brown sticky solid (Yield: 90%, 2.18 g). The compound was used directly in the next step without further purification.

### Step 2. Synthesis of Compound (234)

3-amino-4-fluoro-phenol 233 (2.18 g, 17.1 mmol, 1.0 eq) was added to a molten mixture of di-tert-butyl dicarbonate (3.74 g, 17.1 mmol, 1.0 eq) and indium (III) chloride (38 mg, 0.17 mmol, 0.01 eq). The reaction mixture was stirred at 40° C. for 4 hours. After cooling down, flash chromatography on silica gel was performed using cyclohexane and ethyl acetate to afford expected compound 234 (Yield: 61%, 2.38 g) as off-white solid.

### Step 3. Synthesis of Compound (235)

Tert-butyl N-(2-fluoro-5-hydroxy-phenyl)carbamate 234 (1.96 g, 8.6 mmol, 1.0 eq)) in 85 mL of DMF was added potassium tert-butylate (1.26 g, 11.22 mmol, 1.3 eq). After stirring 30 min at room temperature, 4-chloro-2-amino-3-nitropyridine (1.5 g, 8.6 mmol, 1.0 eq) was added and the mixture was heated at 70° C. for 4 h. The crude was purified by flash chromatography on silica gel was performed using cyclohexane and ethyl acetate to afford expected compound 235 (Yield: 71%, 2.24 g) as yellow sticky solid.

### Step 4. Synthesis of Compound (236)

Palladium on activated charcoal (around 300 mg) was added to a solution of tert-butyl N-[5-[(2-amino-3-nitro-4-pyridyl)oxy]-2-fluoro-phenyl]carbamate 235 (2.24 g, 6.15 mmol, 1.0 eq) in ethanol (50 mL). The reaction mixture was hydrogenated at room temperature over 1 atmosphere of hydrogen for 20 h. The reaction mixture was filtrated through a short pad of celite and rinsed with ethanol. The filtrate was evaporated to dryness to afford the expected compound 236 as red sticky solid (Yield: 90%, 1.86 g).

### Step 5. Synthesis of Compound (237)

Tert-butyl N-[5-[(2,3-diamino-4-pyridyl)oxy]-2-fluoro-phenyl]carbamate 236 (2.02 g, 6.04 mmol, 1.0 eq) in methanol was added dropwise to a solution of glyoxylic acid monohydrate (5.56 g, 60.4 mmol, 10.0 eq) in methanol (100 mL). The mixture was stirred at room temperature for 18 h. The expected compound 237 was obtained as white solid after filtration (Yield: 60%, 1.34 g).

### Step 6. Synthesis of Compound (238)

Tert-butyl-N-[2-fluoro-5-[(3-oxo-4H-pyrido[2,3-b]pyrazin-8-yl)oxy]phenyl]carbamate 237 (1.34 g, 3.6 mmol, 1.0 eq) was treated with 4N HCl in dioxane (29 mL) and the reaction mixture was stirred at room temperature for 4 h. The precipitate was filtered, washed with diethyl ether and dried under vacuo to afford expected compound 238 as brown powder (Yield: 99%, 1.11 g).

### Synthesis of 240: 2,2,2-trichloroethyl (3-(tert-butyl)-1-(4-(3-(dimethylamino)propoxy)phenyl)-1H-pyrazol-5-yl)carbamate

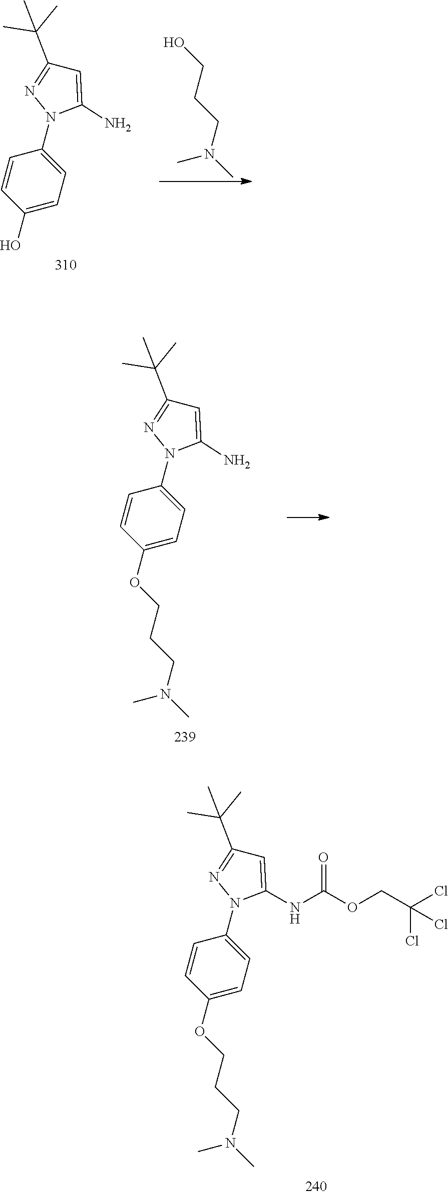

### Step 1. Synthesis of Compound (239)

Diisopropyl azodicarboxylate (0.85 mL, 4.3 mmol, 2.0 eq) was added at room temperature to a solution of 310 (500 mg, 2.16 mmol, 1.0 eq), 3-(dimethylamino)propan-1-ol (0.30 mL, 2.6 mmol, 1.2 eq) and triphenylphosphine (1.13 g, 4.3 mmol, 2.0 eq) in 12 mL of THF. The reaction mixture was stirred at room temperature for 20 h. A 10% solution of citric acid was added, and the mixture was extracted with ether. The aqueous phase was carefully basified with potassium carbonate and extracted with ethyl acetate. The organic phases were dried over magnesium sulfate, filtered and evaporated. The residue was purified by flash chromatography over silica gel using dichloromethane and ammoniac 7N in methanol to afford the expected compound 239 as yellow oil (Yield: 91%, 620 mg).

### Step 2. Synthesis of Compound (240)

At 0° C., 5-tert-butyl-2-[4-[3-(dimethylamino)propoxy]phenyl]pyrazol-3-amine 239 (620 mg, 1.96 mmol, 1.0 eq) was added dropwise dry pyridine (1.1 eq) followed by 2,2,2-trichloroethylchloroformate (0.30 mL, 2.15 mmol, 1.1 eq). The reaction mixture was stirred at room temperature for 18 h. Water was added, and the reaction mixture was extracted with ethyl acetate. The organic layers were combined, dried over magnesium sulfate, filtered and evaporated. The compound 240 was obtained as orange oil (1.07 g, quantitative yield) and was used without purification in the next step.

### Synthesis of 246: 8-(4-amino-3-(methylthio)phenoxy)-2H-pyrido[3,2-b][1,4]oxazin-3(4H)-one Hydrochloride

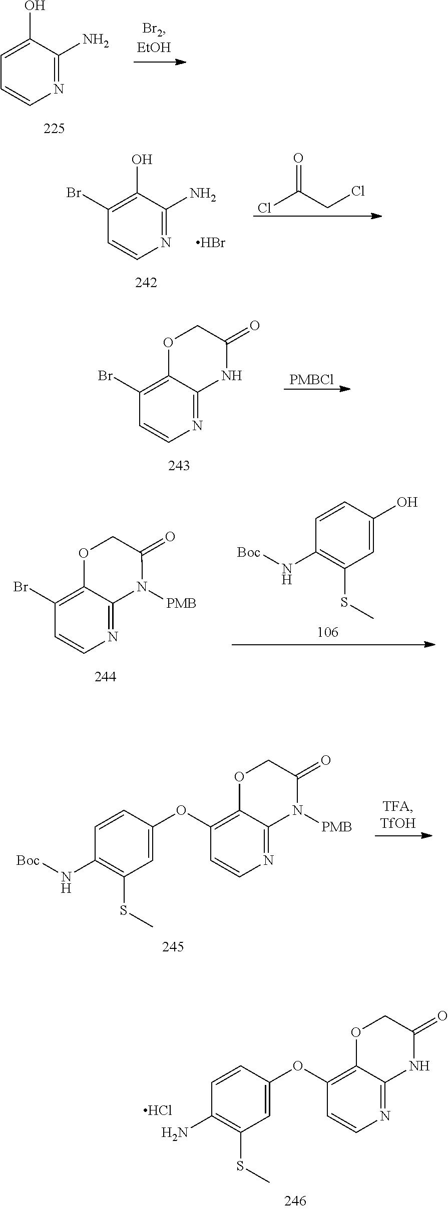

### Step 1. Synthesis of Compound (242)

To a solution of 2-amino-2-hydroxypyridine 241 (10.0 g, 91 mmol) in absolute ethanol (50 mL) at 0° C. was dropwise added bromine (5.2 ml, 102 mmol) and the mixture was stirred at room temperature for 3 days. The solvent was removed under reduced pressure and low temperature. The residue was cooled to 0° C. AcOEt (100 mL) was added and the mixture was stirred for 1 h. The suspension was filtered off (washing with AcOEt). The red-brown solid recovered was dried under reduced pressure leading to the expected product 242 (Yield: 74%, 18.2 g).

### Step 2. Synthesis of Compound (243)

In a sealed tube at 0° C., sodium bicarbonate (2.34 g, 27.9 mmol) was added to a solution of 2-amino-4-bromo-pyridin-3-ol 242 (2.50 g, 9.3 mmol) in a mixture of 2-butanone and water (1:1, 18.6 mL). After 10 min, chloroacetyl chloride (0.74 mL, 9.3 mmol) was added. The mixture was stirred for 3 h at 0° C. Then, the mixture was heated at 80° C. for 18 h. After cooling and filtration of the suspension, the dark-brown solid recovered was dried under vacuum leading to the expected product (Yield: 62%, 1.34 g).

### Step 3. Synthesis of Compound (244)

To a solution of 8-bromo-4H-pyrido[3,2-b][1,4]oxazin-3-one 243 (1.13 g, 4.9 mmol) in DMF (25 mL) were added cesium carbonate (3.22 g, 9.9 mmol) followed by 4-methoxybenzyl chloride (1.16 g, 7.4 mmol) and the mixture was stirred for 18 h at room temperature. After filtration, the solvent was removed in vacuo. The residue was solubilized in AcOEt (100 mL) and washed with water (50 mL) and NH₄Cl sat. (50 mL). The organic fraction was dried over MgSO₄, filtered and concentrated in vacuo. The crude obtained was purified by flash chromatography (40 g, SiO₂, cyclohexane/AcOEt 100:0→40:60, 10 CV) leading to the expected product 244 (Yield: 78%, 1.35 g) as a white solid.

### Step 4. Synthesis of Compound (245)

8-bromo-4-[(4-methoxyphenyl)methyl]pyrido[3,2-b][1,4]oxazin-3-one 244 (350 mg, 1.0 mmol), tert-butyl N-(4-hydroxy-2-methylsulfanyl-phenyl) carbamate 106 (306 mg, 1.2 mmol), K₃PO₄ (764 mg, 3.6 mmol), and tBuXPhos Pd G3 (79 mg, 0.1 mmol) in anhydrous toluene (10 mL) were reacted as described under General Procedure B (120° C., 2 h) to furnish after flash chromatography (40 g, SiO₂, cyclohexane/AcOEt 100:0→50:50, 10 CV) the expected product 245 (Yield: 77%, 403 mg) as a beige foam.

### Step 5. Synthesis of Compound (246)

To a solution of tert-butyl N-{4-[4-{(4-methoxyphenyl)methyl}-3-oxo-pyrido[3,2-b][1,4] oxazin-8-yl]oxy-2-methylsulfanyl-phenyl}carbamate 245 (200 mg, 0.38 mmol) in dichloromethane (38 mL) was added trifluoroacetic acid (2.90 mL, 38 mmol) and trifluoromethanesulfonic acid (1.35 mL, 15 mmol). The reaction mixture was stirred at room temperature for 90 min, diluted with methanol (3 mL) and the pH was adjusted to 8-9 by addition of sat. aq. NaHCO₃. The aqueous layer was extracted with dichloromethane (2×20 mL). The combined organic fractions were dried over MgSO₄, filtered and concentrated under reduced pressure to furnish the expected product 246 (170 mg, quantitative yield) as a beige powder.

### Synthesis of 247: 5-((4-aminonaphthalen-1-yl)oxy)-3,4-dihydro-1,8-naphthyridin-2(1H)-one

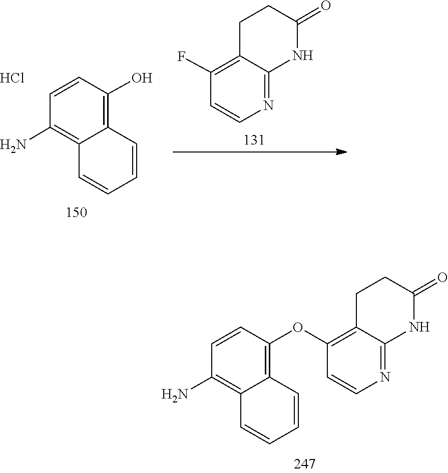 [SMILES: Cl ; Nc1ccc(O)c2ccccc12 ; O=C1CCc2c(F)ccnc2N1 ; Nc1ccc(Oc2ccnc3c2CCC(=O)N3)c2ccccc12]

In a sealed tube, tBuOK (445 mg, 3.97 mmol) followed by 5-fluoro-3,4-dihydro-1H-1,8-naphthyridin-2-one 131 (300 mg, 1.80 mmol) were added at RT to a solution of 4-aminonaphthol hydrochloride 150 (530 mg, 2.70 mmol) in dry DMA (15 mL). The reaction mixture was stirred at 120° C. for 2 h. After cooling to room temperature, water (50 mL), aq. sat. NH₄Cl (50 mL) and AcOEt (100 mL) were added. The aqueous layer was extracted with AcOEt (2×30 mL). The combined organic fractions were dried over MgSO₄, filtered and concentrated in vacuo. The crude obtained was purified by flash chromatography (40 g, SiO₂, cyclohexane/AcOEt 100:0→0:100, 10 CV and AcOEt/CH₃OH 100:0→95:5, 5 CV) leading to the expected product 247 (80 mg, 68% LC/MS purity) as a dark powder.

### Synthesis of 252: Ethyl (4-((4-aminonaphthalen-1-yl)oxy)pyridin-2-yl)carbamate

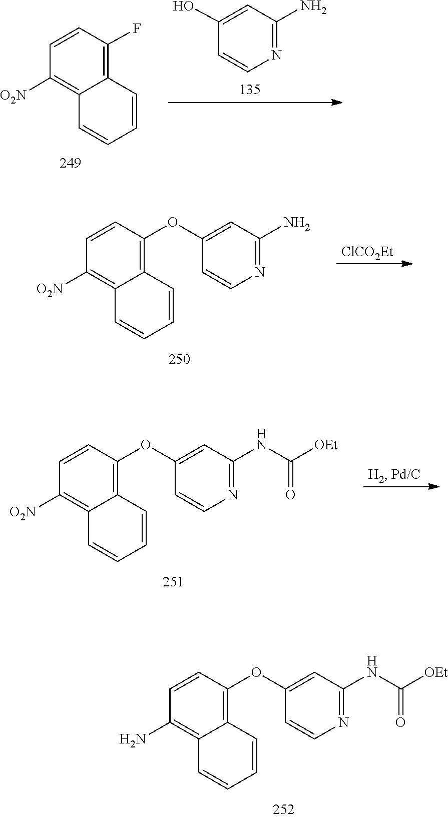

### Step 1. Synthesis of Compound (250)

In a sealed tube, DBU (1.63 mL, 10.9 mmol), 2-aminopyridin-4-ol 135 (0.87 g, 7.9 mmol) in dry acetonitrile (0.1M). The mixture was stirred at room temperature for 30 min before addition of 1-fluoro-4-nitronaphthalene 249 (1.16 g, 6.1 mmol). The reaction mixture was stirred at 80° C. for 2 h. After extraction and concentration the crude was purified by flash chromatography (80 g, SiO₂, cyclohexane/AcOEt 100:0→0:100, 10 CV) leading to the expected product 250 (Yield: 65%, 1.11 g) as an orange solid.

### Step 2. Synthesis of Compound (251)

A stirred solution of 4-[(4-nitro-1-naphthyl)oxy]pyridin-2-amine 250 (562 mg, 2.0 mmol) in anhydrous THF (10 mL) was treated at 0° C. with lithium bis(trimethylsilyl)amide 1.0M solution in THF (3.2 mL, 3.2 mmol). After 15 min at 0° C., a solution of ethyl chloroformate (0.23 mL, 2.4 mmol) in THF (10 mL) was added. The mixture was allowed to warm to room temperature and stirred for 4 h. The reaction was quenched with sat. aq. NH₄Cl and extracted with AcOEt. The organic fractions were dried over MgSO₄, filtered and concentrated in vacuo. The solid obtained was purified by flash chromatography (40 g, SiO₂, cyclohexane/AcOEt 100:0→40:60, 10 CV) leading to the expected product 251 (Yield: 14%, 100 mg) as a yellow solid.

### Step 3. Synthesis of Compound (252)

A suspension of ethyl N-{4-[(4-nitro-1-naphthyl)oxy]-2-pyridyl]carbamate 251 (100 mg, 0.28 mmol) and Pd/C (20 mg) in a mixture ethanol/dichloromethane (1:1; 20 mL) was stirred under hydrogen (1 atm). After 18 h at room temperature and work-up, the expected product 252 (Yield: 85%, 78 mg) was recovered as an orange foam.

### Synthesis of 256: Ethyl (4-(4-amino-3-(methylthio)phenoxy)pyridin-2-yl)carbamate Hydrochloride

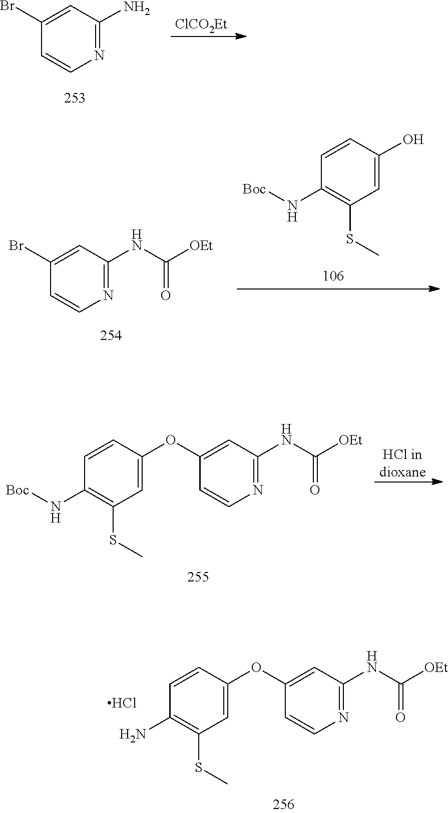

### Step 1. Synthesis of Compound (254)

A stirred solution of 4-bromopyridin-2-amine 253 (1.73 g, 10 mmol) in anhydrous THF (20 mL) was treated at 0° C. with lithium bis(trimethylsilyl)amide 1.0M solution in THF (21.0 mL, 21.0 mmol). After 15 min at 0° C., a solution of ethyl chloroformate (1.30 g, 12.0 mmol) in THF (20 mL) was added. The mixture was allowed to warm to room temperature and stirred overnight. The reaction was quenched with sat. aq. NH₄Cl and extracted with AcOEt. The organic fractions were dried over MgSO₄, filtered and concentrated in vacuo. The solid obtained was triturated in ether and filtered off leading to the expected product 254 (Yield: 33%, 819 mg) as a brown solid.

### Step 2. Synthesis of Compound (255)

In a sealed tube, a suspension of ethyl N-(4-bromo-2-pyridyl)carbamate 254 (245 mg, 1.0 mmol), tert-butyl N-(4-hydroxy-2-methylsulfanyl-phenyl) carbamate 106 (306 mg, 1.2 mmol) in anhydrous toluene (10 mL) was degassed under argon for 15 mn at RT. Then K₃PO₄ (764 mg, 3.6 mmol), and tBuXPhos Pd G3 (79 mg, 0.1 mmol) was added. The mixture was stirred and heated (100° C., 18 h). After cooling and concentration under vacuum, the crude obtained was diluted in AcOEt and washed with water. The organic layer was dried over MgSO₄, filtered and concentrated in vacuo. The residue obtained was purified by flash chromatography (40 g, SiO₂, cyclohexane/AcOEt 100:0→70:30, 10 CV) to obtain the expected product 255 in mixture with unreacted phenol. The batch was used without further purification for next step.

### Step 3. Synthesis of Compound (256)

Ethyl N-{4-[4-(tert-butoxycarbonylamino)-3-methylsulfanyl-phenoxy]-2-pyridyl}carbamate 255 (100 mg, 0.24 mmol) was treated with 4N HCl in dioxane (10 mL) and stirred at room temperature for 2 h. The solvent was removed under vacuum. The beige powder recovered was directly used for next step without any purification.

### Synthesis of 263: 8-(3-amino-2-fluorophenoxy)pyrido[2,3-b]pyrazin-3(4H)-one Hydrochloride

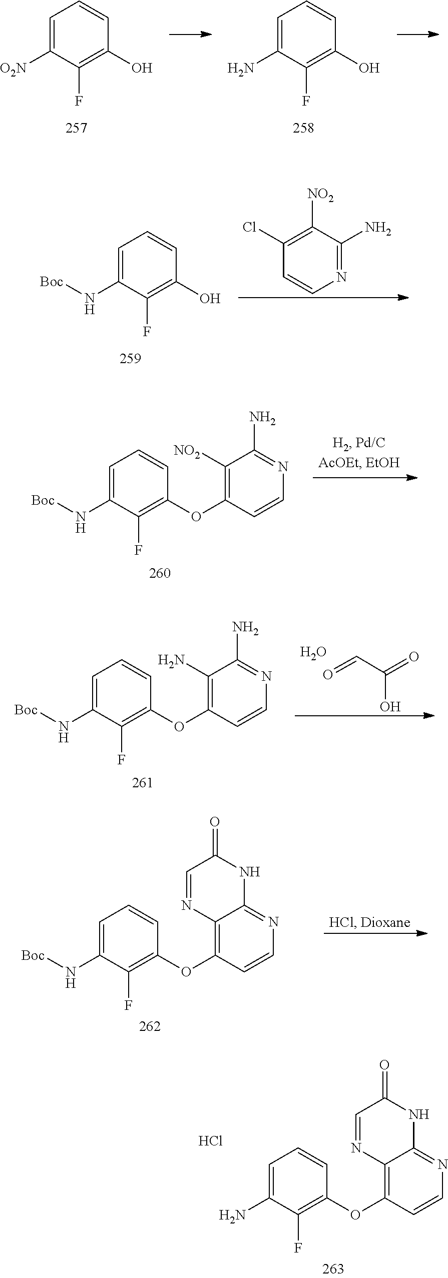

### Step 1. Synthesis of Compound (258)

Palladium on activated charcoal (around 300 mg) was added to a solution of 2-fluoro-3-nitro-phenol 257 (3.0 g, 19.0 mmol, 1.0 eq) in methanol (100 mL). The reaction mixture was hydrogenated at room temperature over 1 atmosphere of hydrogen for 18 h. The reaction mixture was filtrated through a short pad of celite and rinsed with methanol. The filtrate was evaporated to dryness to afford the expected compound 258 as brown solid (Yield: 95%, 1.89 g). The compound was used directly in the next step without further purification.

### Step 2. Synthesis of Compound (259)

3-amino-2-fluoro-phenol 258 (300 mg, 2.36 mmol, 1.0 eq) was added to a molten mixture of di-tert-butyl dicarbonate (515 mg, 2.36 mmol, 1.0 eq) and indium (III) chloride (6 mg, 0.02 mmol, 0.01 eq). The reaction mixture was stirred at 40° C. for 4 h. After cooling down, flash chromatography on silica gel was performed using cyclohexane and ethyl acetate to afford expected compound 259 (600 mg) with 80% of LC/MS purity.

### Step 3. Synthesis of Compound (260)

Tert-butyl N-(2-fluoro-3-hydroxy-phenyl)carbamate 259 (800 mg, 3.52 mmol, 1.0 eq) in 40 mL of DMF was added potassium tert-butylate (514 mg, 4.57 mmol, 1.3 eq). After stirring 30 min at room temperature, 4-chloro-2-amino-3-nitropyridine (611 mg, 3.52 mmol, 1.0 eq) was added and the mixture was heated (60° C., 4 h). The crude was purified by flash chromatography on silica gel was performed using cyclohexane and ethyl acetate to afford expected compound 260 (Yield: 41%, 530 mg) as yellow solid.

### Step 4. Synthesis of Compound (261)

Palladium on activated charcoal (around 100 mg) was added to a solution of tert-butyl N-[3-[(2-amino-3-nitro-4-pyridyl)oxy]-2-fluoro-phenyl]carbamate 260 (950 mg, 2.6 mmol, 1.0 eq) in ethanol (150 mL). The reaction mixture was hydrogenated at room temperature over 1 atmosphere of hydrogen for 20 h. The reaction mixture was filtrated through a short pad of celite and rinsed with methanol. The filtrate was evaporated to dryness to afford the expected compound 261 as black oil (900 mg, quantitative yield).

### Step 5. Synthesis of Compound (262)

Tert-butyl N-[3-[(2,3-diamino-4-pyridyl)oxy]-2-fluoro-phenyl]carbamate 261 (900 mg, 2.69 mmol, 1.0 eq) in methanol was added dropwise to a solution of glyoxylic acid monohydrate (2.5 g, 26.9 mmol, 10.0 eq) in methanol (50 mL). The mixture was stirred at room temperature for 18 h. The expected compound 262 was obtained as beige solid after filtration (Yield: 19%, 190 mg).

### Step 6. Synthesis of Compound (263)

Tert-butyl-N-[2-fluoro-3-[(3-oxo-4H-pyrido[2,3-b]pyrazin-8-yl)oxy]phenyl]carbamate 262 (190 mg, 0.51 mmol, 1.0 eq)) was treated with 4N HCl in dioxane (4.2 mL) and the reaction mixture was stirred at room temperature for 45 min. The precipitate was filtered, washed with diethyl ether and dried under vacuo to afford expected compound 263 as brown powder (170 mg, quantitative yield).

### Synthesis of 266: Benzyl (4-(4-amino-3-(methylthio)phenoxy)pyridin-2-yl)carbamate Hydrochloride

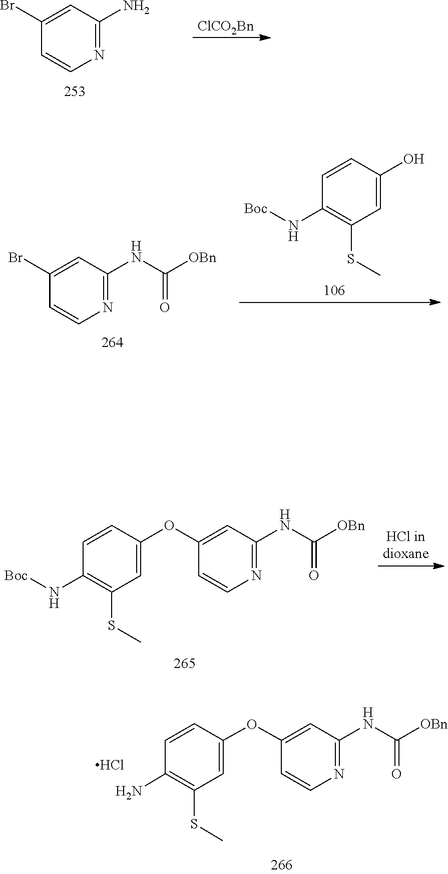

### Step 1. Synthesis of Compound (264)

A stirred solution of 4-bromopyridin-2-amine 253 (1.73 g, 10 mmol) in anhydrous THF (20 mL) was treated at 0° C. with lithium bis(trimethylsilyl)amide 1.0M solution in THF (21.0 mL, 21.0 mmol). After 15 min at 0° C., a solution of benzyl chloroformate (1.70 ml, 12.0 mmol) in THF (20 mL) was added. The mixture was allowed to warm to room temperature and stirred overnight. The reaction was quenched with sat. aq. NH₄Cl and extracted with AcOEt. The organic fractions were dried over MgSO₄, filtered and concentrated in vacuo. The solid obtained was triturated in ether and filtered off leading to the expected product 264 (Yield: 64%, 1.41 g) as a beige solid.

### Step 2. Synthesis of Compound (265)

In a sealed tube, a suspension of benzyl N-(4-bromo-2-pyridyl)carbamate 264 (307 mg, 1.0 mmol), tert-butyl N-(4-hydroxy-2-methylsulfanyl-phenyl) carbamate 106 (306 mg, 1.2 mmol) in anhydrous toluene (10 mL) was degassed under argon for 15 mn at RT. Then K₃PO₄ (764 mg, 3.6 mmol), and tBuXPhos Pd G3 (79 mg, 0.1 mmol) was added. The mixture was stirred and heated (100° C., 18 h). After cooling and concentration under vacuum, the crude obtained was diluted in AcOEt and washed with water. The organic layer was dried over MgSO₄, filtered and concentrated in vacuo. The residue obtained was purified by flash chromatography (40 g, SiO₂, cyclohexane/AcOEt 100:0→70:30, 10 CV) the expected product 265 in mixture with unreacted phenol. The batch was used without further purification for next step.

### Step 3. Synthesis of Compound (266)

Tert-butyl N-[4-{[2-(benzyloxycarbonylamino)-4-pyridyl]oxy}-2-methyl sulfanyl-phenyl] carbamate 265 (108 mg, 0.22 mmol) was treated with 4N HCl in dioxane (10 mL) and stirred at room temperature for 2 h. The solvent was removed under vacuum. The beige powder recovered was directly used for next step without any purification.

### Synthesis of 271: 4-((3,4-dihydro-2H-pyrido[3,2-b][1,4]oxazin-8-yl)oxy)-2-(methylthio)aniline Hydrochloride

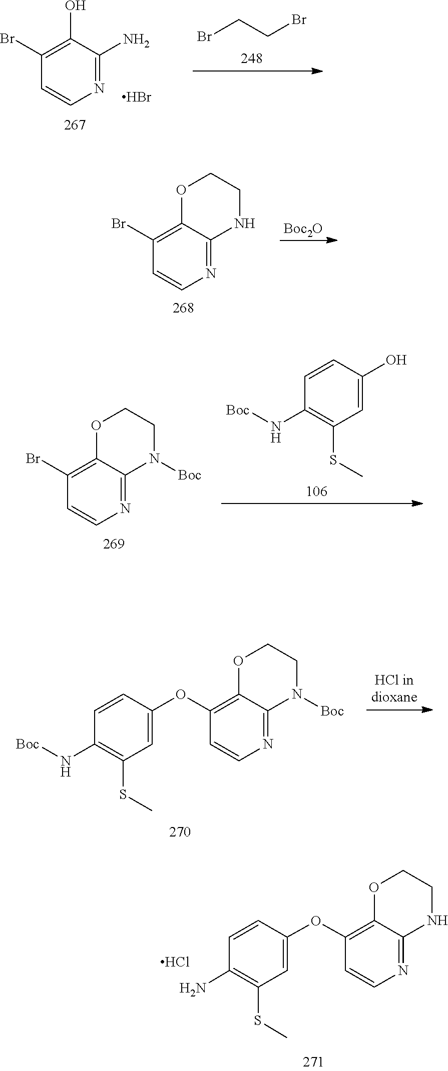

### Step 1. Synthesis of Compound (268)

In a sealed tube containing 2-amino-4-bromo-pyridin-3-ol 267 (2.50 g, 9.3 mmol) in acetonitrile (25 mL) were added cesium carbonate (9.1 g, 27.9 mmol) followed by 1,2-dibromoethane 248 (2.6 g, 13.9 mmol). The mixture was refluxed for 72 h. After cooling and filtration of the suspension, the filtrate was concentrated under reduced pressure. The residue was purified by flash chromatography (40 g, SiO₂, cyclohexane/AcOEt 100:0→0:100, 10 CV followed by AcOEt/CH₃OH 100:0→80:20, 5 CV) leading to the expected product 268 (Yield: 13%, 274 mg) as a beige solid.

### Step 2. Synthesis of Compound (269)

To a solution of 8-bromo-3,4-dihydro-2H-pyrido[3,2-b][1,4]oxazine 268 (330 mg, 1.53 mmol) in anhydrous THF (5 mL) were dropwise added lithium bis(trimethylsilyl)amide 1.0M solution in THF (1.84 mL, 9.9 mmol) followed by di-tert-butyldicarbonate (502 mg; 2.30 mmol). The mixture was stirred at 0° C. for 1 h. The reaction was quenched with sat. aq. NH₄Cl and extracted with AcOEt. The organic fractions were dried over MgSO₄, filtered and concentrated in vacuo. The residue obtained was purified by flash chromatography (40 g, SiO₂, cyclohexane/AcOEt 100:0→50:50, 12 CV) leading to the expected product 269 (Yield: 81%, 395 mg) as a yellow solid.

### Step 3. Synthesis of Compound (270)

In a sealed tube, a suspension of tert-butyl-2,3-dihydropyrido[3,2-b][1,4]oxazine-4-carboxylate 269 (315 mg, 1.0 mmol), tert-butyl N-(4-hydroxy-2-methylsulfanyl-phenyl) carbamate 106 (306 mg, 1.2 mmol) in anhydrous toluene (10 mL) was degassed under argon for 15 mn at RT. Then K₃PO₄ (764 mg, 3.6 mmol) and tBuXPhos Pd G3 (79 mg, 0.1 mmol was added. The mixture was stirred and heated (100° C., 8 h). After cooling and concentration under vacuum, the crude obtained was diluted in AcOEt and washed with water. The organic layer was dried over MgSO₄, filtered and concentrated in vacuo. The residue obtained was purified by flash chromatography (40 g, SiO₂, cyclohexane/AcOEt 100:0→60:40, 10 CV) the expected product 270 (Yield: 32%, 161 mg) as a red oil.

### Step 4. Synthesis of Compound (271)

Tert-butyl 8-[4-(tert-butoxycarbonylamino)-3-methylsulfanyl-phenoxy]-2,3-dihydropyrido [3,2-b][1,4]oxazine-4-carboxylate 270 (160 mg, 0.33 mmol) was treated with 4N HCl in dioxane (10 mL) at room temperature for 24 h. The suspension obtained was filtered off, washed with ether, and dried under vacuum. The beige powder recovered (100 mg) was directly used for next step without any purification

### Synthesis of 273: 2,2,2-trichloroethyl (3-(tert-butyl)isoxazol-5-yl)carbamate

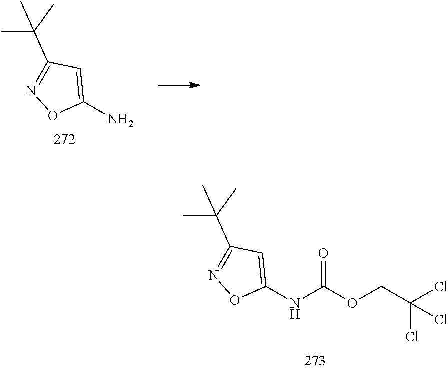

At 0° C., to a solution of 3-tert-butylisoxazol-5-amine 272 (5.0 g, 35.7 mmol, 1.0 eq) in dry THF (0.2M) was added dropwise dry pyridine (1.1 eq) followed by 2,2,2-trichloroethylchloroformate (5.4 mL, 39.2 mmol, 1.1 eq). The reaction mixture was stirred at room temperature for 5 h. Water was added, and the reaction mixture was extracted with ethyl acetate. The organic layers were combined, dried over magnesium sulfate, filtered and evaporated. The residue was purified by flash chromatography over silica gel using cyclohexane and ethyl acetate to afford the expected compound 273 as yellow pale powder (11.5 g, quantitative yield).

### Synthesis of 275: 2,2,2-trichloroethyl (5-(tert-butyl)isoxazol-3-yl)carbamate

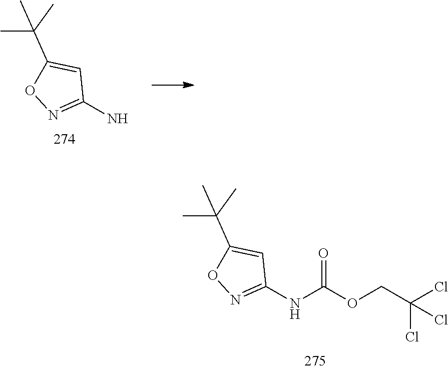

At 0° C., to a solution of 5-tert-butylisoxazol-3-amine (1.0 g, 7.13 mmol, 1.0 eq) in dry THF (0.2M) was added dropwise dry pyridine (1.1 eq) followed by 2,2,2-trichloroethylchloroformate (1.1 mL, 7.8 mmol, 1.1 eq). The reaction mixture was stirred at room temperature for 24 h. Water was added, and the reaction mixture was extracted with ethyl acetate. The organic layers were combined, dried over magnesium sulfate, filtered and evaporated. The residue was obtained as yellow pale powder (2.26 g, quantitative yield) and was used without purification in the next step.

### Synthesis of 279: 2,2,2-trichloroethyl (3-(tert-butyl)-1-(4-(morpholinomethyl)phenyl)-1H-pyrazol-5-yl)carbamate

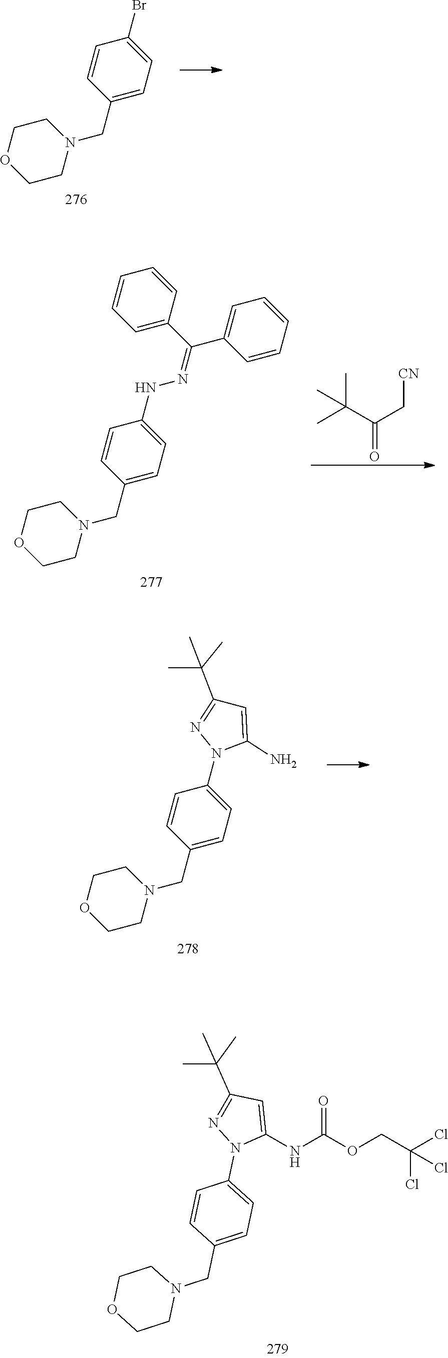

### Step 1. Synthesis of Compound (277)

In a sealed tube, 4-[(4-bromophenyl)methyl]morpholine 276 (2.0 g, 7.8 mmol), benzophenone hydrazone 83 (1.53 g, 7.8 mmol, 1.0 eq), sodium tertiobutylate (1.1 g, 11.71 mmol, 1.5 eq) were put in suspension in 100 mL of toluene. The mixture was degassed with argon for 30 min before addition of BINAP (97 mg, 0.156 mmol, 0.02 eq) and palladium acetate (26 mg, 0.117 mmol, 0.015 eq). The mixture was heated at 100° C. for 18 h. After cooling down, the precipitate was filtered and rinsed with ethyl acetate and methanol. The filtrate was adsorbed on silica gel and purified by flash chromatography using cyclohexane and ethyl acetate to afford the expected compound 277 as yellow foam (Yield: 53%, 1.53 g)

### Step 2. Synthesis of Compound (278)

To a solution of N-(benzhydrylideneamino)-4-(morpholinomethyl)aniline 277 (1.2 g, 3.28 mmol, 1.0 eq) and pivaloyl acetonitrile (616 mg, 4.92 mmol, 1.5 eq) in 20 mL of ethanol wad added hydrochloric acid 12N (2.7 mL, 32.8 mmol, 10.0 eq). The mixture was stirred at reflux for 20 h. Water was added and the aqueous layer was basified with a saturated solution of sodium carbonate (pH 8) and extracted with ethyl acetate. The organic layers were dried over magnesium sulfate, filtered and evaporated under vacuo. The residue was purified by flash chromatography on silica gel using cyclohexane and ethyl acetate to afford the expected compound 278 (Yield: 78%, 802 mg).

### Step 3. Synthesis of Compound (279)

At 0° C., to a solution of 5-tert-butyl-2-[4-(morpholinomethyl)phenyl]pyrazol-3-amine 278 (1.69 g, 5.37 mmol, 1.0 eq) was added dropwise dry pyridine (1.1 eq) followed by 2,2,2-trichloroethylchloroformate (0.81 mL, 5.91 mmol, 1.1 eq). The reaction mixture was stirred at room temperature for 24 h. Water was added, and the reaction mixture was extracted with ethyl acetate. The organic layers were combined, dried over magnesium sulfate, filtered and evaporated. The residue was purified by flash chromatography over silica gel using cyclohexane and ethyl acetate to afford expected compound 279 as white powder (Yield: 81%, 2.13 g).

### Synthesis of 281: N-(4-((4-aminonaphthalen-1-yl)oxy)pyridin-2-yl)acetamide

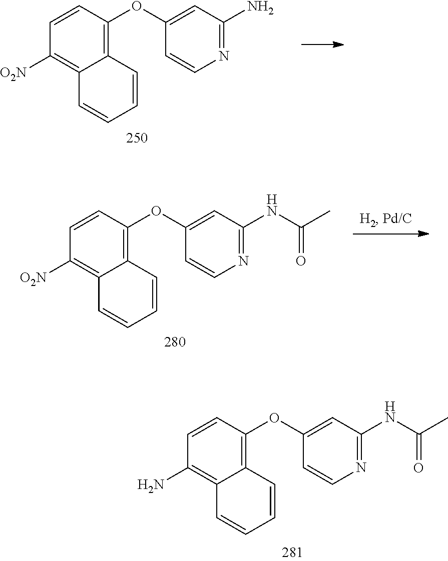 [SMILES: Nc1cc(Oc2ccc([N+](=O)[O-])c3ccccc23)ccn1 ; CC(=O)Nc1cc(Oc2ccc([N+](=O)[O-])c3ccccc23)ccn1 ; CC(=O)Nc1cc(Oc2ccc(N)c3ccccc23)ccn1]

### Step 1. Synthesis of Compound (280)

A stirred solution containing 4-[(4-nitro-1-naphthyl)oxy]pyridin-2-amine 250 (250 mg, 0.89 mmol), propylphosphonic anhydride (1.60 ml, 2.67 mmol), and DIEA (3.10 mL, 20 mmol) in DCE (10 mL) was heated at 80° C. in a sealed tube for 20 h. After cooling at room temperature, the reaction mixture was concentrated in vacuo. The crude obtained was purified by flash chromatography (40 g, SiO₂, cyclohexane/AcOEt 100:0→0:100, 10 CV) leading to the expected product 280 (345 mg) as a yellow solid.

### Step 1. Synthesis of Compound (281)

A suspension of N-{4-[(4-nitro-1-naphthyl)oxy]-2-pyridyl]acetamide 280 (345 mg, 1.07 mmol) and Pd/C (50 mg) in a mixture ethanol/dichloromethane (1:1; 100 mL) was stirred under hydrogen (1 atm). After 18 h at room temperature and work-up, the expected product 281 (Yield: 80%, 250 mg) was recovered as a dark purple solid.

### Synthesis of 283: 2-(methylthio)-4-(pyridin-4-yloxy)aniline

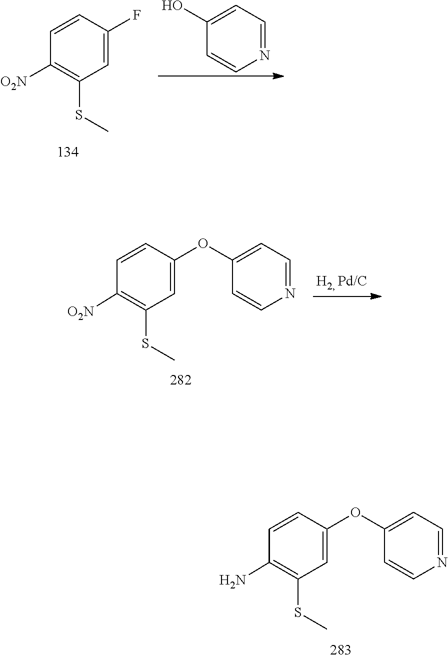

### Step 1. Synthesis of Compound (282)

In a sealed tube, DBU (0.72 mL, 4.81 mmol) was added at room temperature to a solution of 4-hydroxypyridine (330 mg, 3.47 mmol) in dry acetonitrile (0.1M). The mixture was stirred at RT for 30 min before addition of 4-fluoro-2-methylsulfanyl-1-nitro-benzene 134 (500 mg, 2.67 mmol). The reaction mixture was stirred at 80° C. for 2 h. After cooling to room temperature, the suspension was filtered off. The powder was washed with AcOEt and ether, leading after drying to the expected product 282 (Yield: 92%, 645 mg) as a yellow solid.

### Step 2. Synthesis of Compound (283)

A suspension of 4-(3-methylsulfanyl-4-nitro-phenoxy)pyridine 282 (645 mg, 2.46 mmol) and Pd/C (100 mg) in ethanol (50 mL) and dichloromethane (50 mL) was stirred under hydrogen (1 atm). After 18 h at room temperature and work-up, the expected product 283 (570 mg, quantitative yield) was recovered as a brown gum.

### Synthesis of 285: N-(4-(4-amino-3-(methylthio)phenoxy)pyridin-2-yl)methanesulfonamide

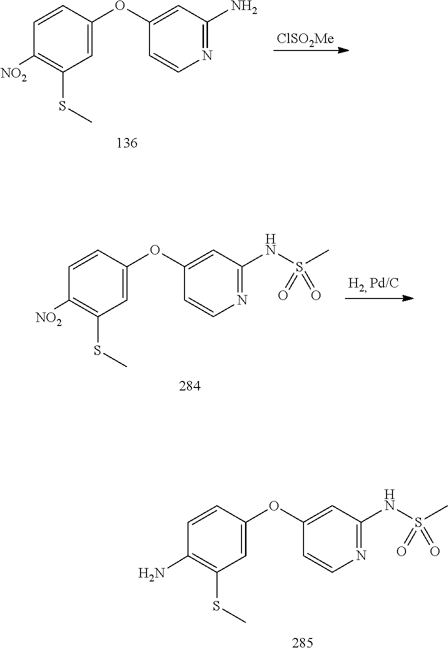 [SMILES: CSc1cc(Oc2ccnc(N)c2)ccc1[N+](=O)[O-] ; CSc1cc(Oc2ccnc(NS(C)(=O)=O)c2)ccc1[N+](=O)[O-] ; "ClSO2Me" ; CSc1cc(Oc2ccnc(NS(C)(=O)=O)c2)ccc1N]

### Step 1. Synthesis of Compound (284)

To a stirred solution of 4-(3-methylsulfanyl-4-nitro-phenoxy)pyridin-2-amine 136 (400 mg, 1.44 mmol) in dichloromethane (40 mL) were slowly added at 0° C. triethylamine (0.60 mL, 4.30 mmol), and methylchloride (0.13 mL, 1.73 mmol). The reaction mixture was stirred at room temperature for 18 h and concentrated in vacuo. The crude obtained was purified by flash chromatography (40 g, SiO₂, dichloromethane/AcOEt 100:0→50:50, 10 CV) leading to the expected product 284 (90 mg) as a yellow solid.

### Step 1. Synthesis of Compound (285)

A suspension of N-[4-(3-methylsulfanyl-4-nitro-phenoxy)-2-pyridyl]methanesulfonamide 284 (42 mg, 0.12 mmol) and Pd/C (20 mg) in a mixture ethanol/dichloromethane (2:1, 15 mL) was stirred under hydrogen (1 atm). After 4 h at room temperature and work-up, the expected product 285 (41 mg, quantitative) was recovered as a yellow powder.

### Synthesis of 287: 7-(4-amino-3-(methylthio)phenoxy)-1H-imidazo[4,5-b]pyridin-2(3H)-one Hydrochloride

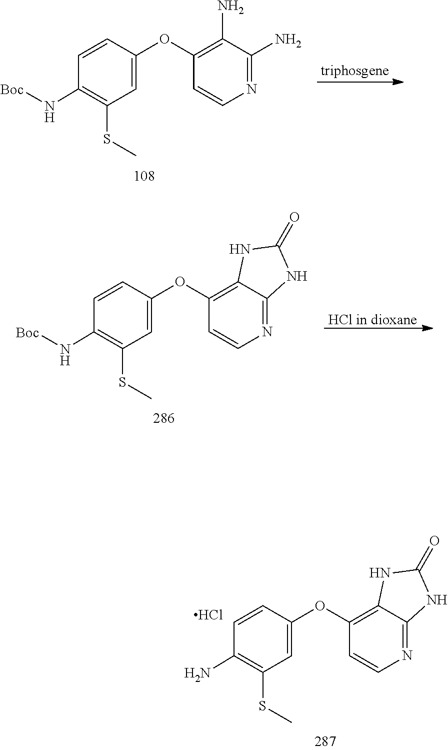 [SMILES: CSc1cc(Oc2ccnc(N)c2N)ccc1NC(=O)OC(C)(C)C ; "triphosgene" ; CSc1cc(Oc2ccnc3[nH]c(=O)[nH]c23)ccc1NC(=O)OC(C)(C)C ; "HCl in dioxane" ; Cl ; CSc1cc(Oc2ccnc3[nH]c(=O)[nH]c23)ccc1N]

### Step 1. Synthesis of Compound (286)

In a sealed tube, a solution of tert-butyl N-{4-[(2,3-diamino-4-pyridyl)oxy]-2-methylsulfanyl-phenyl}carbamate 108 (450 mg, 1.24 mmol) and pyridine (1.0 mL, 12.4 mmol) in dry THF (11 mL) was treated dropwise at 0° C. by a solution of triphosgene (368 mg, 1.24 mmol) in THF (4 mL). The reaction mixture was stirred at room temperature for 18 h. After evaporation of the solvent, the crude solid obtained was washed successively with water and diethylether. The brown solid recovered after drying was dissolved in dry THF (15 mL). Pyridine (1.0 mL, 12.4 mmol) was added and the mixture was heated at 70° C. for 48 h. After cooling at room temperature and evaporation in vacuo, water (30 mL) and AcOEt (50 mL) were added. The aqueous layer was extracted with AcOEt (2×30 mL). The combined organic fractions were dried over MgSO₄, filtered and concentrated in vacuo. The crude obtained was purified by flash chromatography (40 g, SiO₂, cyclohexane/AcOEt 100:0→0:100, 10 CV) leading to the expected product 286 (436 mg, 54% LC/MS purity) as a brown solid.

### Step 2. Synthesis of Compound (287)

Tert-butyl N-{2-methylsulfanyl-4-[(2-oxo-1,3-dihydroimidazo[4,5-b]pyridin-7-yl)oxy]phenyl} carbamate 286 (436 mg, 1.12 mmol) was treated with 4N HCl in dioxane (10 mL) at room temperature for 2 h. The suspension obtained was filtered off, washed with ether, and dried under vacuum. The brown powder recovered (Yield: 93%, 337 mg) was directly used for next step without any purification.

### Synthesis of 296: 8-(4-amino-2-(methylthio)phenoxy)pyrido[2,3-b]pyrazin-3(4H)-one Hydrochloride

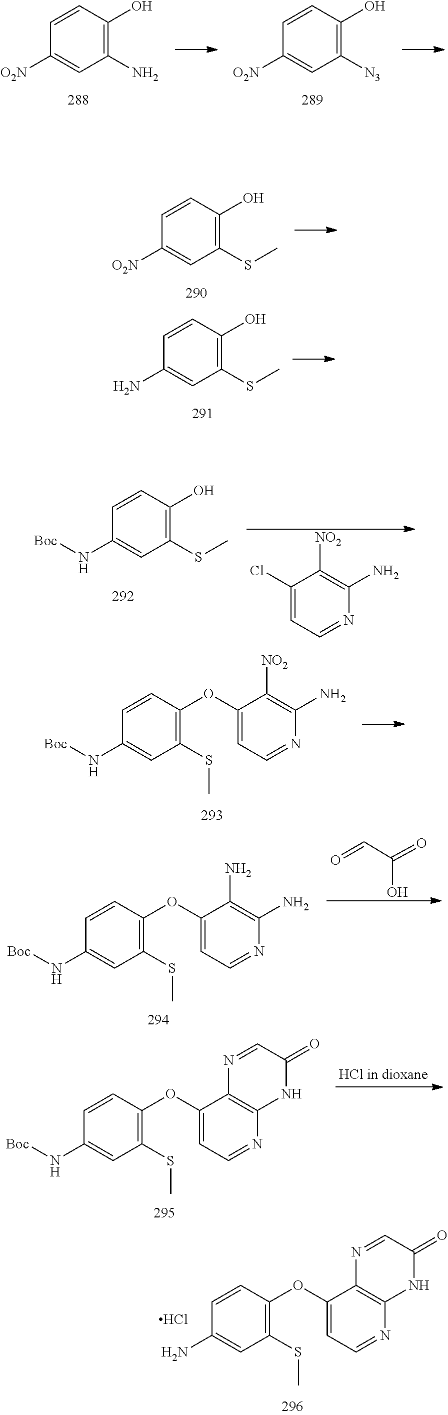

### Step 1. Synthesis of Compound (289)

A solution of sodium nitrite (2.24 g, 32.4 mmol) in water (12 mL) was added dropwise at 0° C. to a solution of 2-amino-4-nitro-phenol 288 (5.0 g, 32.4 mmol) and tetrafluoroboric acid in water (48% wt., 12 mL). The mixture was stirred 1 h at room temperature. The precipitate formed was filtered off, washed with water, and triturated in acetone. The white solid recovered was dried leading to the expected product 289 (Yield: 73%, 4.25 g).

### Step 2. Synthesis of Compound (290)

Copper (730 mg, 11.5 mmol) was added at 0° C. to a suspension of 2-azido-4-nitro-phenol 289 (4.25 g, 22.9 mmol) in water (100 mL), followed by sodium thiomethoxide (3.22 g, 45.9 mmol) in water (100 mL). After 18 h at room temperature, the reaction mixture is filtered over Celite. The filtrate was acidified by addition of HCl 1 N. The precipitate formed was filtered off, washed with water, and dried under vacuum leading to a black solid (Yield: 77%, 3.25 g) corresponding to the expected product 290.

### Step 3. Synthesis of Compound (291)

Sodium hydrosulfite (7.6 g, 43.7 mmol) was added at 50° C. to a solution of 2-methylsulfanyl-4-nitro-phenol 290 (3.25 g, 17.5 mmol) in NaOH 1N (52 mL, 52 mmol). The reaction mixture was stirred at 100° C. for 4 h. The precipitate formed was filtered off to recover a brown solid (Yield: 26%, 700 mg) corresponding to the expected product 291.

### Step 4. Synthesis of Compound (292)

Indium trichloride (10 mg, 0.05 mmol) and Boc₂O (986 mg, 4.mmol) in dry THF (3 mL) were stirred at 40° C. during 15 min. Then, 4-amino-2-methylsulfanyl-phenol 291 (700 mg, 4.5 mmol) was added and the mixture heated at 40° C. for 18 h. After cooling, the crude was purified by flash chromatography (40 g, SiO₂, cyclohexane/AcOEt 100:0→0:100, 10 CV) leading to the expected product 292 (Yield: 87%, 1.0 g) as a brown solid.

### Step 5. Synthesis of Compound (293)

Potassium tert-butoxide (569 mg, 5.1 mmol) was added at room temperature to a solution of tert-butyl N-(4-hydroxy-3-methylsulfanyl-phenyl)carbamate 292 (1.0 g, 3.9 mmol) in DMF (20 ml). After 15 min, 4-chloro-3-nitro-pyridin-2-amine (680 mg, 3.9 mmol) was added. The reaction mixture was heated at 80° C. for 3 h. After cooling at room temperature, the crude obtained was diluted in AcOEt and washed with water. The organic layer was dried over MgSO₄, filtered and concentrated in vacuo. The residue obtained was purified by flash chromatography (40 g, SiO₂, cyclohexane/AcOEt 100:0→80:20, 10 CV) leading to the expected product 293 (Yield: 85%, 1.3 g) as a yellow solid.

### Step 6. Synthesis of Compound (294)

A suspension of tert-butyl N-{4-[(2-amino-3-nitro-4-pyridyl)oxy]-3-methylsulfanyl-phenyl}carbamate 293 (1.3 g, 3.31 mmol) and Pd/C (120 mg) in ethanol (50 mL) was stirred under hydrogen (3 bars). After 20 h at room temperature, work-up, and purification by flash chromatography (40 g, SiO₂, cyclohexane/AcOEt 100:0→0:100, 10 CV) the expected product 294 (Yield: 58%, 700 mg) was recovered as a brown oil.

### Step 7. Synthesis of Compound (295)

Tert-butyl N-{4-[(2,3-diamino-4-pyridyl)oxy]-3-methylsulfanyl-phenyl} carbamate 294 (450 mg, 1.24 mmol)) in methanol was added dropwise to a solution of glyoxylic acid (1.15 g, 12.4 mmol) in methanol (10 mL). After stirring 18 h at room temperature, work-up, and purification by flash chromatography (40 g, SiO₂, cyclohexane/AcOEt 100:0→0:100, 10 CV) the expected product 295 (Yield: 14%, 70 mg) was recovered as a white solid.

### Step 8. Synthesis of Compound (296)

Tert-butyl N-{3-methylsulfanyl-4-[(3-oxo-4H-pyrido[2,3-b]pyrazin-8-yl)oxy]phenyl} carbamate 295 (220 mg, 0.73 mmol) was treated with HCl 4N in dioxane (4 mL) at room temperature for 1 h30. The suspension was filtered off, washed with dioxane, and dried under vacuum leading to a yellow solid (Yield: 73%, 160 mg) corresponding to the expected product 296.

### Synthesis of 298: 5-(4-amino-3-(methylthio)phenoxy)-1,8-naphthyridin-2(1H)-one

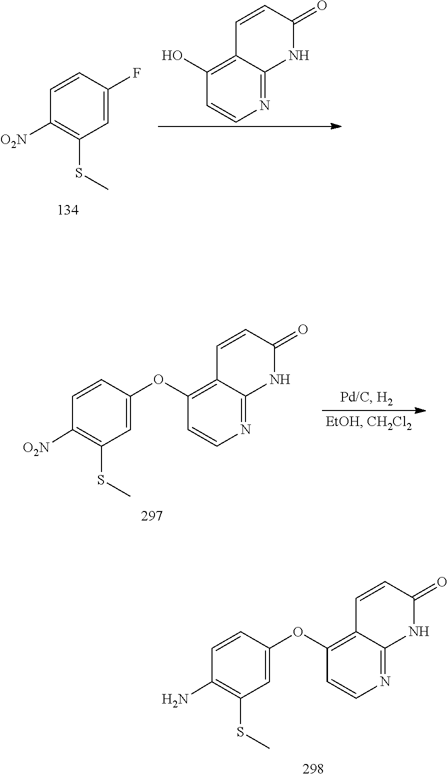

### Step 1. Synthesis of Compound (297)

Potassium tert-butoxide (389 mg, 3.45 mmol, 1.3 eq) was added to a solution of 5-hydroxy-1H-1,8-naphthyridin-2-one (430 mg, 2.65 mmol, 1.0 eq) in 10 mL of DMF. After stirring 30 minutes at room temperature, 4-fluoro-2-methylsulfanyl-1-nitro-benzene 134 (496 mg, 2.65 mmol, 1.0 eq) was added. The solution was stirred at 120° C. for 3 days. After cooling down, the solvent was evaporated and the residue was purified by flash chromatography over silica gel using cyclohexane and ethyl acetate to afford expected compound 297 as a mixture used in the next step (Yield: 13%, 113 mg) as a brown oil.

### Step 2. Synthesis of Compound (298)

Palladium on activated charcoal (around 60 mg) was added to a solution of 5-(3-(methylthio)-4-nitrophenoxy)-1,8-naphthyridin-2(1H)-one 297 (113 mg, 0.34 mmol) in ethanol and dichloromethane (15 mL/5 mL). The reaction mixture was hydrogenated at 30° C. over 1 atmosphere of hydrogen for 20 h. The reaction mixture was filtrated through a short pad of celite and rinsed with ethanol. The filtrate was evaporated to afford the expected compound 298 (101 mg, quantitative) as brown solid.

### Synthesis of 303: 2,2,2-trichloroethyl (3-(tert-butyl)-1-(4-((dimethylamino)methyl)phenyl)-1H-pyrazol-5-yl)carbamate

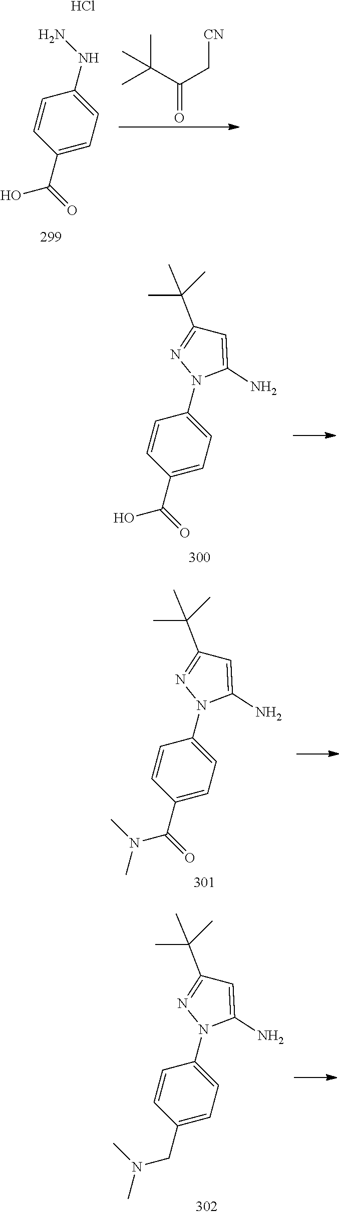

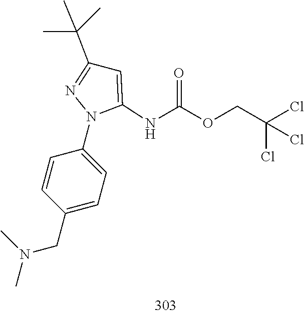 [SMILES: CN(C)Cc1ccc(-n2nc(C(C)(C)C)cc2NC(=O)OCC(Cl)(Cl)Cl)cc1]

### Step 1. Synthesis of Compound (300)

4-Hydrazinobenzoic acid hydrochloride 299 (3.0 g, 19.7 mmol, 1.0 eq) and pivaloyl acetonitrile (2.7 g, 21.7 mmol, 1.1 eq in ethanol wad added hydrochloric acid 12 N (10.0 eq). The mixture was stirred at reflux for 18 h. After cooling down, the reaction mixture was evaporated to dryness to afford expected compound 300 as orange foam (5.6 g, quantitative yield).

### Step 2. Synthesis of Compound (301)

EDCI hydrochloride (1.6 g, 8.27 mmol, 1.3 eq) was added to a solution of 4-(5-amino-3-tert-butyl-pyrazol-1-yl)benzoic acid 300 (1.65 g, 6.36 mmol, 1.0 eq) and dimethyl amine 2M in methanol (4.8 mL, 9.5 mmol, 1.5 eq) in 20 mL of THF. After 3 h at room temperature, the reaction mixture was concentrated, taken up in dichloromethane and washed twice with brine. The organic phase was dried over magnesium sulfate, filtered and evaporated to afford the expected compound 301 (Yield: 80%, 1.45 g).

### Step 3. Synthesis of Compound (302)

1M solution of boron trifluoride diethyl etherate in THF (40.5 mL, 40.5 mmol, 8.0 eq) was slowly added to a solution of 4-(5-amino-3-tert-butyl-pyrazol-1-yl)-N,N-dimethyl-benzamide 301 (1.45 g, 5.1 mmol, 1.0 eq) in 50 mL of dry THF. The reaction mixture was stirred at room temperature for 4 h and was carefully quenched with methanol. The residue was dissolved several times in methanol and evaporated to be purified by flash chromatography over silica gel using cyclohexane and ethyl acetate and methanol to afford expected compound 302 (Yield: 38%, 528 mg).

### Step 4. Synthesis of Compound (303)

To a mixture of 5-tert-butyl-2-[4-[(dimethylamino)methyl]phenyl]pyrazol-3-amine 302 (528 mg, 1.94 mmol, 1.0 eq) and DI EA (6.0 equiv.) in acetonitrile (0.1M) was added 2,2,2-trichloroethylchloroformate (0.29 mL, 2.13 mmol, 1.1 eq). The reaction mixture was stirred at room temperature for 18 h. Water was added, and the reaction mixture was extracted with ethyl acetate. The organic layers were combined, dried over magnesium sulfate, filtered and evaporated. The residue was used directly in the next step (1.03 g, 69% by LC/MS).

### Synthesis of 305: N-(4-(4-amino-3-(methylthio)phenoxy)pyridin-2-yl)-2-((tert-butyldimethylsilyl)oxy)acetamide

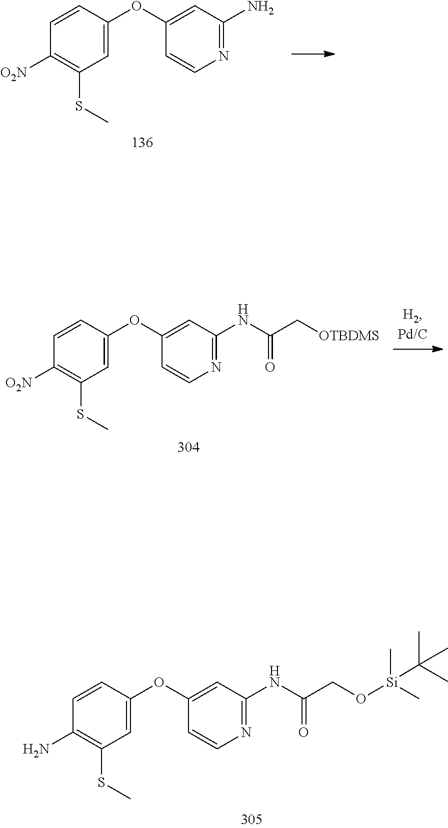 [SMILES: CSc1cc(Oc2ccnc(N)c2)ccc1[N+](=O)[O-] ; CSc1cc(Oc2ccnc(NC(=O)CO[Si](C)(C)C(C)(C)C)c2)ccc1[N+](=O)[O-] ; CSc1cc(Oc2ccnc(NC(=O)CO[Si](C)(C)C(C)(C)C)c2)ccc1N]

### Step 1. Synthesis of Compound (304)

A stirred solution containing 4-(3-methylsulfanyl-4-nitro-phenoxy)pyridin-2-amine 136 (890 mg, 3.21 mmol), 2-[tert-butyl(dimethyl]silyl]oxyacetamide (611 mg, 3.1 mmol), propylphosphonic anhydride (5.70 mL, 9.63 mmol), and DIEA (1.70 mL, 9.63 mmol) in DCE was heated at 80° C. in a sealed tube for 18 h. After cooling at RT, NaHCO₃ sat (30 mL) was added. The aqueous layer was extracted with dichloromethane (2×30 mL). The combined organic fractions were dried over MgSO₄, filtered and concentrated in vacuo. The crude obtained was purified by flash chromatography (80 g, SiO₂, cyclohexane/AcOEt 100:0→60:40, 10 CV) leading to the expected product 304 (Yield: 24%, 347 mg) as a yellow solid.

### Step 2. Synthesis of Compound (305)

A suspension of 2-[tert-butyl(dimethyl)silyl]oxy-N-[4-(3-methylsulfanyl-4-nitro-phenoxy)pyridyl]acetamide 304 (300 mg, 0.94 mmol) and Pd/C (70 mg) in a mixture ethanol/dichloromethane (4:1, 50 mL) was stirred under hydrogen (1 atm). After 18 h at room temperature and work-up, the expected product (Yield: 61%, 228 mg) was recovered as an orange oil.

### Synthesis of 307: 2,2,2-trichloroethyl (3-(tert-butyl)-1-(p-tolyl)-1H-pyrazol-5-yl)carbamate

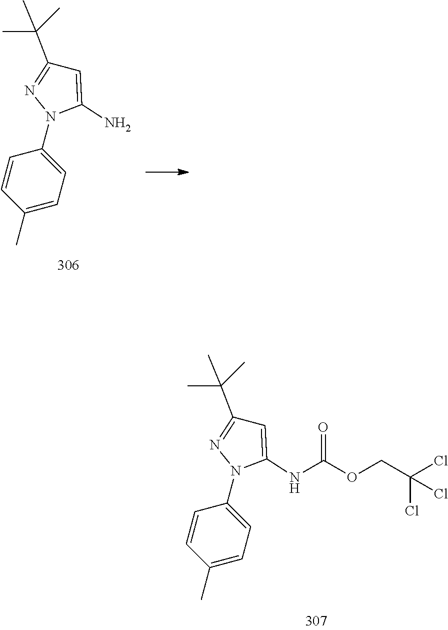 [SMILES: Cc1ccc(-n2nc(C(C)(C)C)cc2N)cc1 ; Cc1ccc(-n2nc(C(C)(C)C)cc2NC(=O)OCC(Cl)(Cl)Cl)cc1]

At 0° C., to a solution of N-[5-tert-butyl-2-(p-tolyl)pyrazol-3-yl]amine 306 (550 mg, 2.4 mmol, 1.0 eq) was added dropwise dry pyridine (1.1 eq) followed by 2,2,2-trichloroethylchloroformate (0.36 mL, 2.64 mmol, 1.1 eq). The reaction mixture was stirred at room temperature for 18 h. Water was added, and the reaction mixture was extracted with ethyl acetate. The organic layers were combined, dried over magnesium sulfate, filtered and evaporated. The residue was obtained as white powder (Yield: 98%, 950 mg) and was used without purification in the next step.

### Synthesis of 310: 4-(5-amino-3-tert-butyl-pyrazol-1-yl)phenol

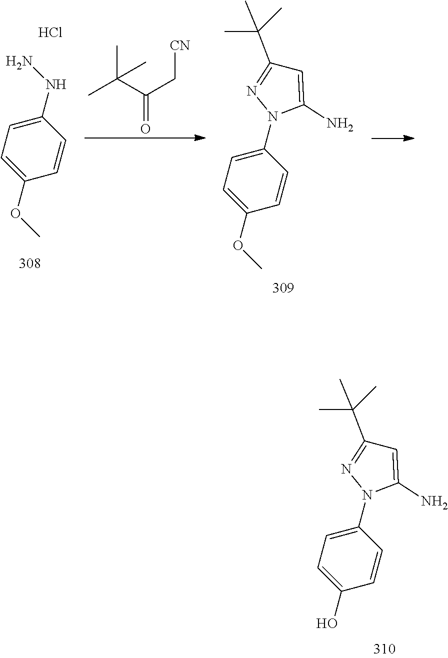

### Step 1. Synthesis of Compound (309)

(4-methoxyphenyl)hydrazine hydrochloride 308 (1.5 g, 8.59 mmol, 1.0 eq) and pivaloyl acetonitrile (1.1 g, 8.59 mmol, 1.0 eq) were reacted at reflux for 18 h. The solution was basified with hydrogencarbonate, filtrated and evaporated. The residue was purified by flash chromatography over silica gel using cyclohexane and ethyl acetate to afford expected compound 309 as orange oil (2.1 g, quantitative yield).

### Step 2. Synthesis of Compound (310)

5-tert-butyl-2-(4-methoxyphenyl)pyrazol-3-amine 309 (2.1 g, 8.56 mmol, 1.0 eq) was solubilized in 30 mL of dichloromethane. Aluminium chloride (5.7 g, 42.8 mmol, 5.0 eq) was added and the reaction mixture was heated at reflux for 3 days. After cooling down, the mixture was poured in water and dichloromethane and the organic layer was washed with water and brine, dried over magnesium sulfate and evaporated. The residue was purified by flash chromatography over silica gel using cyclohexane and ethyl acetate to afford 4-(5-amino-3-tert-butyl-pyrazol-1-yl)phenol 310 as yellow solid (Yield: 35%, 690 mg).

### Synthesis of Specific Compounds

### Synthesis of Compound (a): 1-(2-fluoro-4-((3-oxo-3,4-dihydropyrido[2,3-b]pyrazin-8-yl)oxy)phenyl)-3-(3-methyl-1-phenyl-1H-pyrazol-5-yl)urea

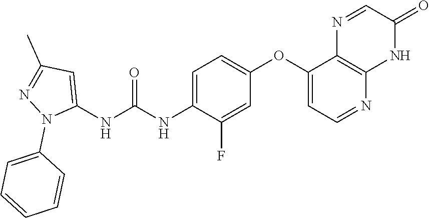 [SMILES: Cc1cc(NC(=O)Nc2ccc(Oc3ccnc4[nH]c(=O)cnc34)cc2F)n(-c2ccccc2)n1]

### Step 1. Synthesis of Compound 1.5

According to Scheme 2 Step 1: Compound 3 (1.62 g, 6.0 mmol, 1.05 eq) was dissolved in MeCN (20 mL) and aminopyrazole 1.1 (1.0 g, 5.77 mmol, 1 eq) was added to the solution. The reaction mixture was stirred at ambient temperature for 12 h, then the reaction mixture was evaporated under reduced pressure. The residue was purified by column chromatography silica gel (DCM) (Yield: 87%, 2.2 g).

### Step 2. Synthesis of Compound 1.6

According to Scheme 2 Step 2: Et₃N.3HF (8.1 mL, 50 mmol, 10 eq) was added to the solution of compound 1.5 (2.2 g, 5 mmol) in THF (20 mL) and the reaction mixture was stirred at ambient temperature for 12 h. The solvent was removed under reduced pressure, water was added to the residue, formed precipitate was filtered off, washed with water and dried to give product (Yield: 70%, 1.15 g).

### Step 3. Synthesis of Compound 1.7

According to Scheme 2 Step 3: Compound 1.6 (1.15 g, 3.52 mmol, 1 eq) was added to the suspension of NaH (60%, 150 mg, 3.7 mmol, 1.05 eq) in DMSO (10 mL) and the reaction mixture was stirred at ambient temperature for 0.5 h. Then 4-chloro-3-nitropyridin-2-amine (610 mg, 3.52 mmol, 1.0 eq) was added and the mixture was stirred at 60° C. overnight. Then the mixture was cooled down, water (50 mL) was added, the product was extracted with EtOAc (3×50 mL), the organic layers were dried under Na₂SO₄ and evaporated under reduced pressure. The residue was purified residue was purified by column chromatography on silica gel (DCM→EtOAc) (Yield: 68%, 1.12 g).

### Step 4. Synthesis of Compound 1.8

According to Scheme 2 Step 4: A mixture of compound 1.7 (1.12 g, 2.42 mmol, 1 eq) and 10% Pd/C (10% by weight, 0.12 g) in MeOH (20 mL) was stirred at room temperature under flow of H₂ for 12 h. Then Pd/C was filtered, the solvent was evaporated under reduced pressure. The residue was used directly to the next step without additional purification (Yield: 95%, 1.0 g).

### Step 5. Synthesis of Compound 1.9: 1-(2-fluoro-4-((3-oxo-3,4-dihydropyrido[2,3-b]pyrazin-8-yl)oxy)phenyl)-3-(3-methyl-1-phenyl-1H-pyrazol-5-yl)urea (Compound (a))

According to Scheme 2 Step 5: A mixture of compound 1.8 (1.0 g, 2.3 mmol, 1 eq) and ethyl oxoacetate (50% in toluene, 0.7 mL, 3.45 mmol, 1.5 eq) in EtOH (10 mL) was stirred at ambient temperature for 12 h. The solvents were evaporated under reduced pressure, the residue was purified twice by column chromatography (DCM→DCM/MeOH 98:2→DCM/MeOH/NH₃ 98:2:0.1) to afford compound (a) (Yield: 0.5%, 5 mg). MS m/z (ES) [M+H]⁺: 472.10

### Synthesis of Compound (b): 1-(2-fluoro-4-((3-oxo-3,4-dihydropyrido[2,3-b]pyrazin-8-yl)oxy)phenyl)-3-(3-isopropyl-1-phenyl-1H-pyrazol-5-yl)urea

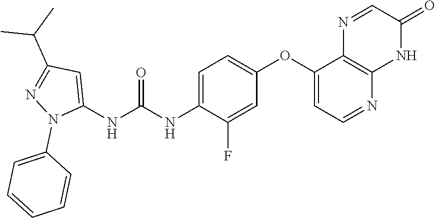 [SMILES: CC(C)c1cc(NC(=O)Nc2ccc(Oc3ccnc4[nH]c(=O)cnc34)cc2F)n(-c2ccccc2)n1]

### Step 1. Synthesis of Compound 1.5

According to Scheme 2 Step 1: Compound 3 (1.46 g, 5.47 mmol, 1.1 eq) was dissolved in MeCN (20 mL) and aminopyrazole 1.1 (1.0 g, 4.97 mmol, 1 eq) was added to the solution. The reaction mixture was stirred at ambient temperature for 12 h, then the reaction mixture was evaporated under reduced pressure. The residue was purified by column chromatography silica gel (DCM) (Yield: 82%, 1.9 g).

### Step 2. Synthesis of Compound 1.6

According to Scheme 2 Step 2: Et₃N.3HF (6.6 mL, 40.5 mmol, 10 eq) was added to the solution of compound 1.5 (1.9 g, 4.05 mmol) in THF (20 mL) and the reaction mixture was stirred at ambient temperature overnight. The solvent was removed under reduced pressure, water was added to the residue, formed precipitate was filtered off, washed with water and dried to give product (Yield: 70%, 1.0 g).

### Step 3. Synthesis of Compound 1.7

According to Scheme 2 Step 3: Compound 1.6 (1 g, 2.82 mmol, 1 eq) was added to the suspension of NaH (60%, 130 mg, 3.1 mmol, 1.1 eq) in DMSO (10 mL) and the reaction mixture was stirred at ambient temperature for 0.5 h. Then 4-chloro-3-nitropyridin-2-amine (510 mg, 2.96 mmol, 1.05 eq) was added and the mixture was stirred at 60° C. for 12 h. Then the mixture was cooled down, water (50 mL) was added, the product was extracted with EtOAc (3×50 mL), the organic layers were dried under Na₂SO₄ and evaporated under reduced pressure. The residue was purified residue was purified by column chromatography on silica gel (DCM→EtOAc) (Yield: 44%, 600 mg).

### Step 4. Synthesis of Compound 1.8

According to Scheme 2 Step 4: A mixture of compound 1.7 (600 mg, 1.22 mmol, 1 eq) and 10% Pd/C (10% by weight, 60 mg) in MeOH (20 mL) was stirred at room temperature under flow of H₂ for 12 h. Then Pd/C was filtered, the solvent was evaporated under reduced pressure. The residue was used directly to the next step without additional purification (560 mg, quantitative yield).

### Step 5. Synthesis of Compound 1.9: 1-(2-fluoro-4-((3-oxo-3,4-dihydropyrido[2,3-b]pyrazin-8-yl)oxy)phenyl)-3-(3-isopropyl-1-phenyl-1H-pyrazol-5-yl)urea (Compound (b))

According to Scheme 2 Step 5: A mixture of compound 1.8 (560 mg, 1.22 mmol, 1 eq) and ethyl oxoacetate (50% in toluene, 0.5 mL, 2.44 mmol, 2 eq) in EtOH (10 mL) was stirred at ambient temperature for 12 h. The solvents were evaporated under reduced pressure, the residue was purified by column chromatography (DCM→DCM/MeOH 98:2→DCM/MeOH/NH₃ 98:2:0.1) to afford compound (b) (Yield: 2.6%, 16 mg). MS m/z (ES) [M+H]⁺: 500.20

### Synthesis of Compound (c): 1-(3-(sec-butyl)-1-phenyl-1H-pyrazol-5-yl)-3-(2-fluoro-4-((3-oxo-3,4-dihydropyrido[2,3-b]pyrazin-8-yl)oxy)phenyl)urea

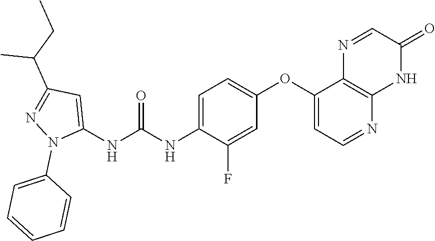 [SMILES: CCC(C)c1cc(NC(=O)Nc2ccc(Oc3ccnc4[nH]c(=O)cnc34)cc2F)n(-c2ccccc2)n1]

### Step 1. Synthesis of Compound 1.5

According to Scheme 2 Step 1: Compound 3 (1.37 g, 5.11 mmol, 1.1 eq) was dissolved in MeCN (20 mL) and aminopyrazole 1.1 (1.0 g, 4.64 mmol, 1 eq) was added to the solution. The reaction mixture was stirred at ambient temperature for 12 h, then the reaction mixture was evaporated under reduced pressure. The residue was purified by column chromatography silica gel (DCM) (Yield: 67%, 1.5 g).

### Step 2. Synthesis of Compound 1.6

According to Scheme 2 Step 2: Et₃N.3HF (5.1 mL, 31.1 mmol, 10 eq) was added to the solution of compound 1.5 (1.5 g, 3.11 mmol) in THF (20 mL) and the reaction mixture was stirred at ambient temperature for 12 h. The solvent was removed under reduced pressure, water was added to the residue, formed precipitate was filtered off, washed with water and dried to give product (Yield: 80%, 920 mg).

### Step 3. Synthesis of Compound 1.7

According to Scheme 2 Step 3: Compound 1.6 (920 mg, 2.49 mmol, 1 eq) was added to the suspension of NaH (60%, 110 mg, 2.74 mmol, 1.1 eq) in DMSO (10 mL) and the reaction mixture was stirred at ambient temperature for 30 min. Then 4-chloro-3-nitropyridin-2-amine (450 mg, 2.61 mmol, 1.05 eq) was added and the mixture was stirred at 60° C. for 12 h. Then the mixture was cooled down, water (50 mL) was added, the product was extracted with EtOAc (3×50 mL), the organic layers were dried under Na₂SO₄ and evaporated under reduced pressure. The residue was purified residue was purified by column chromatography on silica gel (DCM→EtOAc) (Yield: 50%, 630 mg).

### Step 4. Synthesis of Compound 1.8

According to Scheme 2 Step 4: A mixture of compound 1.7 (630 mg, 1.25 mmol, 1 eq) and 10% Pd/C (10% by weight, 60 mg) in MeOH (20 mL) was stirred at room temperature under flow of H₂ overnight. Then Pd/C was filtered, the solvent was evaporated under reduced pressure. The residue was used directly to the next step without additional purification (590 mg, quantitative yield).

### Step 5. Synthesis of Compound 1.9: 1-(3-(sec-butyl)-1-phenyl-1H-pyrazol-5-yl)-3-(2-fluoro-4-((3-oxo-3,4-dihydropyrido[2,3-b]pyrazin-8-yl)oxy)phenyl)urea (Compound (c))

According to Scheme 2 Step 5: A mixture of compound 1.8 (590 mg, 1.25 mmol, 1 eq) and ethyl oxoacetate (50% in toluene, 0.5 mL, 2.50 mmol, 2 eq) in EtOH (10 mL) was stirred at ambient temperature for 12 h. The solvents were evaporated under reduced pressure, the residue was purified by column chromatography (DCM→DCM/MeOH 98:2→DCM/MeOH/NH₃ 98:2:0.1) to afford compound (c) (Yield: 10%, 64 mg). MS m/z (ES) [M+H]⁺: 514.30

### Synthesis of Compound (d): 1-(2-fluoro-4-((3-oxo-3,4-dihydropyrido[2,3-b]pyrazin-8-yl)oxy)phenyl)-3-(1-phenyl-3-(1-(trifluoromethyl)cyclopropyl)-1H-pyrazol-5-yl)urea

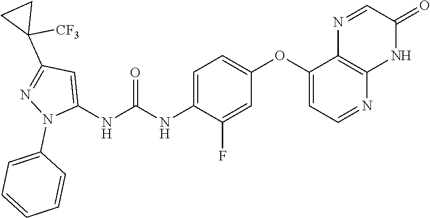 [SMILES: O=C(Nc1ccc(Oc2ccnc3[nH]c(=O)cnc23)cc1F)Nc1cc(C2(C(F)(F)F)CC2)nn1-c1ccccc1]

### Step 1. Synthesis of Compounds 1.2

According to Scheme 1 Step 1: To a mixture of aminopyrazole 14 (200 mg, 0.7 mmol) and DIPEA (0.4 mL, 2.2 mmol) in dichloromethane (5 mL), triphosgene (220 mg, 0.7 mmol) was added at 0° C. and the reaction mixture was stirred for 16 h. Then reaction mixture was evaporated to dryness, the residue was dissolved in THF and precipitate was removed by filtration and the solution was evaporated to dryness. The corresponding isocyanate 1.2 was used in next synthesis without purification.

### Step 2. Synthesis of Compounds 1.4: 1-(2-fluoro-4-((3-oxo-3,4-dihydropyrido[2,3-b]pyrazin-8-yl)oxy)phenyl)-3-(1-phenyl-3-(1-(trifluoromethyl)cyclopropyl)-1H-pyrazol-5-yl)urea (Compound (d))

According to Scheme 1 Step 2: To a solution of compound 1.2 (170 mg, 0.7 mmol) and compound 8 (100 mg, 0.36 mmol) in DMSO (2 mL) was stirred at 60° C. for 12 h. The reaction mixture was cooled and diluted with water. The precipitate was filtrated, washed with ether (100 mL) and dried. The solid after evaporation was subjected to column chromatography on silica gel eluting with dichloromethane/ethyl acetate (0→100%) to afford the compound (d) (Yield: 4%, 7 mg). MS m/z (ES) [M+H]⁺: 566.30

### Synthesis of Compound (e): 1-(3-cyclopropyl-1-phenyl-1H-pyrazol-5-yl)-3-(2-fluoro-4-((3-oxo-3,4-dihydropyrido[2,3-b]pyrazin-8-yl)oxy)phenyl)urea

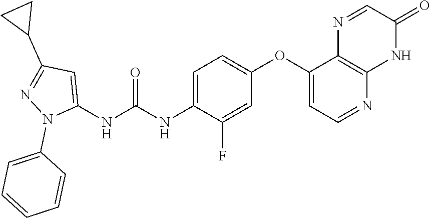 [SMILES: O=C(Nc1ccc(Oc2ccnc3[nH]c(=O)cnc23)cc1F)Nc1cc(C2CC2)nn1-c1ccccc1]

### Step 1. Synthesis of Compounds 1.13

According to Scheme 5 Step 1: Compound 18 (1 g, 4.4 mmol) was dissolved in DCM and (COCl)₂ (830 mg, 6.6 mmol) was added and reaction mixture was stirring for 12 h at 20° C. Then solvent was concentrated in vacuo (Yield: 92%, 1 g).

### Step 2. Synthesis of Compounds 1.14

According to Scheme 5 Step 2: Compound 1.13 (1 g, 4 mmol), NaN₃ (400 mg, 6 mmol) was mixed in Me₂CO/H₂O (40/10 mL). After stirring for 2 h at 20° C., water (200 mL) was added to the reaction mixture, and the resulting mixture was extracted with EtOAc (2×50 mL). The combined organic extracts were washed with brine and dried over Na₂SO₄. The solvent was then removed under reduced pressure (Yield: 97%, 1 g).

### Step 3. Synthesis of Compounds 1.4: 1-(3-cyclopropyl-1-phenyl-1H-pyrazol-5-yl)-3-(2-fluoro-4-((3-oxo-3,4-dihydropyrido[2,3-b]pyrazin-8-yl)oxy)phenyl)urea (Compound (e))

According to Scheme 5 Step 3: A solution of Compound 1.14 (180 mg, 0.7 mmol) in DMSO (2 mL) was stirring for 2 h at 120° C., reaction mixture cooling to room temperature. Compound 8 (100 mg, 0.36 mmol) was added to a reaction mixture. After stirring for 12 h at 100° C., reaction mixture cooling to room temperature, water (25 mL) was added to the reaction mixture, and the resulting mixture was extracted with diethyl ether (50 mL). The crude product was filtered and purified with column using THF as eluent to afford compound (e) (Yield: 40%, 140 mg). MS m/z (ES) [M+H]⁺: 498.30

### Synthesis of Compound (f): 1-(2-fluoro-4-((3-oxo-3,4-dihydropyrido[2,3-b]pyrazin-8-yl)oxy)phenyl)-3-(3-(3-methyloxetan-3-yl)-1-phenyl-1H-pyrazol-5-yl)urea

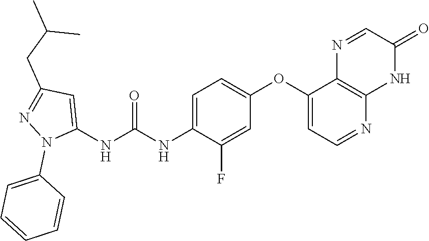 [SMILES: CC(C)Cc1cc(NC(=O)Nc2ccc(Oc3ccnc4[nH]c(=O)cnc34)cc2F)n(-c2ccccc2)n1]

### Step 1. Synthesis of Compounds 1.11

According to Scheme 4 Step 1: To a solution of compound 22 (100 mg, 0.4 mmol), pyridine (70 mg, 0.8 mmol) in THF (5 mL) phenyl chloroformate (72 mg, 0.45 mmol) was added and reaction mixture was stirred at ambient temperature for 2 h. The solvent was evaporated and the residue after evaporation was taken up with EtOAc and water. The organic layer was separated and filtered through anhydrous sodium sulfate, evaporated. The residue after evaporation was subjected to column chromatography on silica gel eluting with dichloromethane/EtOAc (0→10%) to afford the compound 1.11 (Yield: 92%, 0.14 g).

### Step 2. Synthesis of Compounds 1.4: 1-(2-fluoro-4-((3-oxo-3,4-dihydropyrido[2,3-b]pyrazin-8-yl)oxy)phenyl)-3-(3-(3-methyloxetan-3-yl)-1-phenyl-1H-pyrazol-5-yl)urea (Compound (f))

According to Scheme 4 Step 2: To a mixture of compound 1.11 (140 mg, 0.4 mmol) and triethylamine (0.06 mL, 0.4 mmol) in dioxane (2 mL) a compound 8 (100 mg, 0.36 mmol) was added, reaction mixture was stirred at 10° C. for 12 h. The reaction mixture was cooled and diluted with water. The precipitate was filtrated, washed with ether and dried. The solid was subjected to column chromatography on silica gel eluting with dichloromethane/ethyl acetate (0→100%) to afford the compound (f) (Yield: 9%, 17 mg). MS m/z (ES) [M+H]⁺: 528.30

### Synthesis of Compound (g): 1-(2-fluoro-4-((3-oxo-3,4-dihydropyrido[2,3-b]pyrazin-8-yl)oxy)phenyl)-3-(3-isobutyl-1-phenyl-1H-pyrazol-5-yl)urea

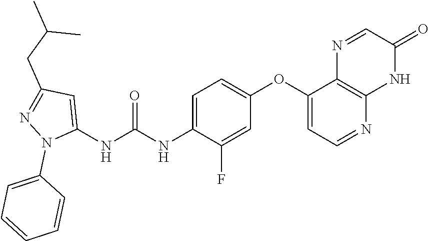 [SMILES: CC(C)Cc1cc(NC(=O)Nc2ccc(Oc3ccnc4[nH]c(=O)cnc34)cc2F)n(-c2ccccc2)n1]

### Step 1. Synthesis of Compound 1.5

According to Scheme 2 Step 1: Compound 3 (2.78 g, 10.4 mmol, 1.1 eq) was dissolved in MeCN (20 mL) and aminopyrazole 1.1 (2.0 g, 9.47 mmol, 1 eq) was added to the solution. The reaction mixture was stirred at ambient temperature for 12 h, then the reaction mixture was evaporated under reduced pressure. The residue was purified by column chromatography silica gel (DCM) (Yield: 77%, 3.5 g).

### Step 2. Synthesis of Compound 1.6

According to Scheme 2 Step 2: Et₃N.3HF (11.2 mL, 72.5 mmol, 10 eq) was added to the solution of compound 1.5 (3.5 g, 7.25 mmol, 1 eq) in THF (30 mL) and the reaction mixture was stirred at ambient temperature for 12 h. The solvent was removed under reduced pressure, water was added to the residue, formed precipitate was filtered off, washed with water and dried to give product 1.6 (Yield: 72%, 1.9 g).

### Step 3. Synthesis of Compound 1.7

According to Scheme 2 Step 3: Compound 1.6 (1.9 g, 5.21 mmol, 1 eq) was added to the suspension of NaH (60%, 230 mg, 5.73 mmol, 1.1 eq) in DMSO (15 mL) and the reaction mixture was stirred at ambient temperature for 30 min. Then 4-chloro-3-nitropyridin-2-amine (950 mg, 5.21 mmol, 1.0 eq) was added and the mixture was stirred at 60° C. for 12 h. Then the mixture was cooled down, water (50 mL) was added, the product was extracted with EtOAc (3×50 mL), the organic layers were dried under Na₂SO₄ and evaporated under reduced pressure. The residue was purified residue was purified by column chromatography on silica gel (DCM→EtOAc) (Yield: 23%, 600 mg).

### Step 4. Synthesis of Compound 1.8

According to Scheme 2 Step 4: A mixture of compound 1.7 (600 mg, 1.19 mmol, 1 eq) and 10% Pd/C (10% by weight, 60 mg) in MeOH (20 mL) was stirred at room temperature under flow of H₂ for 12 h. Then Pd/C was filtered, the solvent was evaporated under reduced pressure. The residue was used directly to the next step without additional purification (Yield: 95%, 540 mg).

### Step 5. Synthesis of Compound 1.9: 1-(2-fluoro-4-((3-oxo-3,4-dihydropyrido[2,3-b]pyrazin-8-yl)oxy)phenyl)-3-(3-isobutyl-1-phenyl-1H-pyrazol-5-yl)urea (Compound (g))

According to Scheme 2 Step 5: A mixture of compound 1.8 (540 mg, 1.13 mmol, 1 eq) and ethyl oxoacetate (50% in toluene, 0.46 mL, 2.26 mmol, 2 eq) in EtOH (10 mL) was stirred at ambient temperature for 12 h. The solvents were evaporated under reduced pressure, the residue was purified twice by column chromatography (DCM→DCM/MeOH 98:2→DCM/MeOH/NH₃ 98:2:0.1) to obtain compound (g) (Yield: 0.85%, 5 mg). MS m/z (ES) [M+H]⁺: 514.30

### Synthesis of Compounds (h) and (o): 1-(1-acetyl-3-(tert-butyl)-1H-pyrazol-5-yl)-3-(2-fluoro-4-((3-oxo-3,4-dihydropyrido[2,3-b]pyrazin-8-yl)oxy)phenyl)urea (Compound (o)); 1-(3-(tert-butyl)-1H-pyrazol-5-yl)-3-(2-fluoro-4-((3-oxo-3,4-dihydropyrido[2,3-b]pyrazin-8-yl)oxy)phenyl)urea (Compound (h))

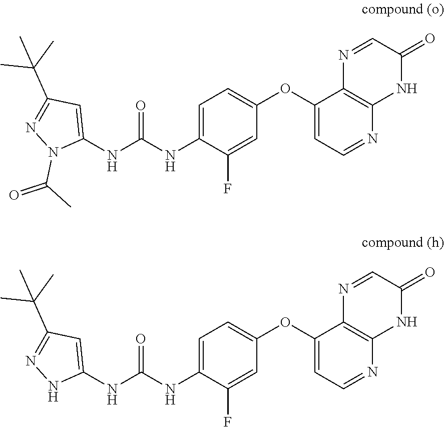 [SMILES: CC(=O)n1nc(C(C)(C)C)cc1NC(=O)Nc1ccc(Oc2ccnc3[nH]c(=O)cnc23)cc1F ; CC(C)(C)c1cc(NC(=O)Nc2ccc(Oc3ccnc4[nH]c(=O)cnc34)cc2F)[nH]n1]

### Step 1. Synthesis of Compound (o): 1-(1-acetyl-3-(tert-butyl)-1H-pyrazol-5-yl)-3-(2-fluoro-4-((3-oxo-3,4-dihydropyrido[2,3-b]pyrazin-8-yl)oxy)phenyl)urea

According to Scheme 1 Step 1 & 2: Triphosgene (0.246 g, 0.8 mmol, 1 eq) was added portions to a solution of the aminopyrazole 24 (0.15 g, 0.83 mmol, 1 eq) and Et₃N (0.32 mL, 2.5 mmol, 3 eq) in DCM (50 mL) at −20° C., the reaction mixture was stirred at ambient temperature for 12 h. Solvent was removed under reduced pressure, THF (50 mL) was added to the residue, the formed precipitate was filtered off, solvent was evaporated under reduced pressure and dissolved in 3 mL DMSO, then compound 8 (160 mg, 0.58 mmol) of was added and the mixture was stirred for 12 h at 50° C. After cooling water was added and precipitate was filtered off and purified by column chromatography silica gel (DCM) to get compound (o) (Yield: 40%, 150 mg). MS m/z (ES) [M+H]⁺: 480.40

### Step 2. Synthesis of Compound (h): 1-(3-(tert-butyl)-1H-pyrazol-5-yl)-3-(2-fluoro-4-((3-oxo-3,4-dihydropyrido[2,3-b]pyrazin-8-yl)oxy)phenyl)urea

100 mg (0.21 mmol) of compound (o) in MeOH (20 mL) were added 5 mL 25% ammonia aqueous solution and stirred 3 h at 20° C. Then mixture evaporated off and purified by column chromatography silica gel (DCM) to obtain compound (h) (Yield: 33%, 30 mg). MS m/z (ES) [M+H]⁺: 438.40

### Synthesis of Compound (i): 1-(3-(tert-butyl)-1-isopropyl-1H-pyrazol-5-yl)-3-(2-fluoro-4-((3-oxo-3,4-dihydropyrido[2,3-b]pyrazin-8-yl)oxy)phenyl)urea

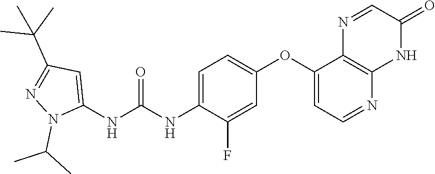 [SMILES: CC(C)n1nc(C(C)(C)C)cc1NC(=O)Nc1ccc(Oc2ccnc3[nH]c(=O)cnc23)cc1F]

### Step 1. Synthesis of Compound 1.2

According to Scheme 1 Step 1: Triphosgene (115 mg, 0.38 mmol, 1 eq) was added portions to a solution of 3-(tert-butyl)-1-isopropyl-1H-pyrazol-5-amine 1.1 (70 mg, 0.38 mmol, 1 eq) and Et₃N (0.13 mL, 1.15 mmol, 3 eq) in DCM (10 mL) at −20° C., the reaction mixture was stirred at ambient temperature for 12 h. Solvent was removed under reduced pressure, THF (50 mL) was added to the residue, the formed precipitate was filtered off, solvent was evaporated under reduced pressure.

### Step 2. Synthesis of Compound 1.4: 1-(3-(tert-butyl)-1-isopropyl-1H-pyrazol-5-yl)-3-(2-fluoro-4-((3-oxo-3,4-dihydropyrido[2,3-b]pyrazin-8-yl)oxy)phenyl)urea (Compound (i))

According to Scheme 1 Step 2: The precipitate 1.2 was dissolved in 3 mL of DMSO, then compound 8 (100 mg, 0.36 mmol) of was added and the mixture was stirred for 12 h at 50° C. After cooling water was added and precipitate was filtered off and purified by column chromatography silica gel (DCM) to afford compound (i) (Yield: 10%, 18 mg). MS m/z (ES) [M+H]⁺: 480.50

¹H-NMR (DMSO-d₆), δ (ppm, J (Hz): 1.20 (s 9H, t-Bu), 1.35 (1, 6H, i-Pr), 4.4 (m, H, i-Pr), 6.05 (s, 1H, H_{pyrazole}) 6.65 (d, J=5.6, 1H_{pyridine}), 7.05 (m, 1H, Hₐᵣₒₘ), 7.40 (m, 1H, Hₐᵣₒₘ), 8.39 (d, J=5.6, 1H, H_{pyridine}), 8.72 (s, 1H, NH), 8.80 (s, 1H, NH), 12.93 (s, 1H, NH).

### Synthesis of Compound (j)): 1-(1-benzyl-3-(tert-butyl)-1H-pyrazol-5-yl)-3-(2-fluoro-4-((3-oxo-3,4-dihydropyrido[2,3-b]pyrazin-8-yl)oxy)phenyl)urea

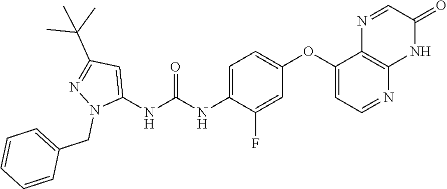 [SMILES: CC(C)(C)c1cc(NC(=O)Nc2ccc(Oc3ccnc4[nH]c(=O)cnc34)cc2F)n(Cc2ccccc2)n1]

### Step 1. Synthesis of Compound 1.5

According to Scheme 2 Step 1: Compound 3 (2.01 g, 7.52 mmol, 1.15 eq) was dissolved in MeCN (20 mL) and 1-benzyl-3-(tert-butyl)-1H-pyrazol-5-amine 1.1 (1.5 g, 6.54 mmol, 1 eq) was added to the solution. The reaction mixture was stirred at ambient temperature for 12 h, then the reaction mixture was evaporated under reduced pressure. The residue was purified by column chromatography silica gel (DCM) (Yield: 86%, 2.8 g).

### Step 2. Synthesis of Compound 1.6

According to Scheme 2 Step 2: Et₃N.3HF (9.18 mL, 56.3 mmol, 10 eq) was added to the solution of compound 1.5 (2.8 g, 5.63 mmol, 1 eq) in THF (30 mL) and the reaction mixture was stirred at ambient temperature for 12 h. The solvent was removed under reduced pressure, water was added to the residue, formed precipitate was filtered off, washed with water and dried to give product 1.6 (Yield: 93%, 2.0 g).

### Step 3. Synthesis of Compound 1.7

According to Scheme 2 Step 3: Compound 1.6 (2.0 g, 5.23 mmol, 1 eq) was added to the suspension of NaH (60%, 0.25 g, 6.27 mmol, 1.2 eq) in DMSO (10 mL) and the reaction mixture was stirred at ambient temperature for 30 min. Then 4-chloro-3-nitropyridin-2-amine (1.04 g, 6.01 mmol, 1.15 eq) was added and the mixture was stirred at 60° C. for 12 h. Then the mixture was cooled down, water (50 mL) was added, the product was extracted with EtOAc (3×50 mL), the organic layers were dried under Na₂SO₄ and evaporated under reduced pressure. The residue was purified residue was purified by column chromatography on silica gel (DCM→EtOAc) (Yield: 22%, 600 mg).

### Step 4. Synthesis of Compound 1.8

According to Scheme 2 Step 4: A mixture of compound 1.7 (500 mg, 0.96 mmol, 1 eq) and 10% Pd/C (10% by weight, 50 mg) in MeOH (10 mL) was stirred at room temperature under flow of H₂ for 12 h. Then Pd/C was filtered, the solvent was evaporated under reduced pressure. The residue was used directly to the next step without additional purification (Yield: 85%, 400 mg).

### Step 5. Synthesis of Compound 1.9: 1-(1-benzyl-3-(tert-butyl)-1H-pyrazol-5-yl)-3-(2-fluoro-4-((3-oxo-3,4-dihydropyrido[2,3-b]pyrazin-8-yl)oxy)phenyl)urea (Compound (j))

According to Scheme 2 Step 5: A mixture of compound 1.8 (400 mg, 0.82 mmol, 1 eq) and ethyl oxoacetate (50% in toluene, 0.33 mL, 1.64 mmol, 2 eq) in EtOH (10 mL) was stirred at ambient temperature for 12 h. The solvents were evaporated under reduced pressure, the residue was purified by column chromatography (DCM→DCM/MeOH 98:2→DCM/MeOH/NH₃ 98:2:0.1) to give compound (j) (Yield: 2.8%, 12 mg). MS m/z (ES) [M+H]⁺: 528.40

### Synthesis of Compound (k): 1-(3-(tert-butyl)-1-(2-morpholinoethyl)-1H-pyrazol-5-yl)-3-(2-fluoro-4-((3-oxo-3,4-dihydropyrido[2,3-b]pyrazin-8-yl)oxy)phenyl)urea

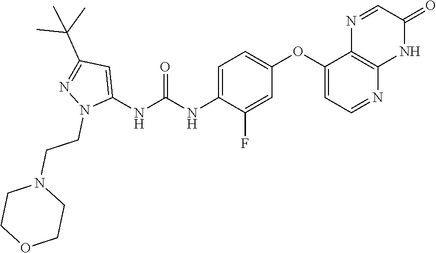 [SMILES: CC(C)(C)c1cc(NC(=O)Nc2ccc(Oc3ccnc4[nH]c(=O)cnc34)cc2F)n(CCN2CCOCC2)n1]

### Step 1. Synthesis of Compound 1.2

According to Scheme 1 Step 1: Triphosgene (115 mg, 0.38 mmol, 1 eq) was added portions to a solution of compound 26 (70 mg, 0.38 mmol, 1 eq) and Et₃N (0.13 mL, 1.15 mmol, 3 eq) in DCM (10 mL) at −20° C., the reaction mixture was stirred at ambient temperature or 12 h. Solvent was removed under reduced pressure, THF (50 mL) was added to the residue, the formed precipitate was filtered off, solvent was evaporated under reduced pressure

### Step 2. Synthesis of Compound 1.4: 1-(3-(tert-butyl)-1-(2-morpholinoethyl)-1H-pyrazol-5-yl)-3-(2-fluoro-4-((3-oxo-3,4-dihydropyrido[2,3-b]pyrazin-8-yl)oxy)phenyl)urea (Compound (k))

According to Scheme 1 Step 2: The previous was dissolved in 3 mL DMSO, then 0.1 g (0.36 mmol) of compound 8 was added and the mixture was stirred for 12 h at 50° C. After cooling water was added and precipitate was filtered off and purified by column chromatography silica gel (DCM) to afford compound (k) (Yield: 2%, 4 mg). MS m/z (ES) [M+H]⁺: 551.30

### Synthesis of Compound (I): 1-(3-(tert-butyl)-1-(pyridin-3-yl)-1H-pyrazol-5-yl)-3-(2-fluoro-4-((3-oxo-3,4-dihydropyrido[2,3-b]pyrazin-8-yl)oxy)phenyl)urea

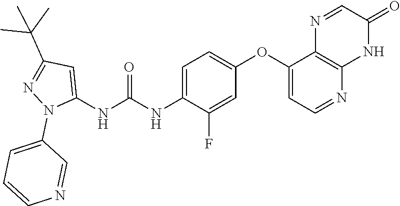 [SMILES: CC(C)(C)c1cc(NC(=O)Nc2ccc(Oc3ccnc4[nH]c(=O)cnc34)cc2F)n(-c2cccnc2)n1]

### Step 1. Synthesis of Compound 1.2

According to Scheme 1 Step 1: To a solution of 3-tert-butyl-1-pyridin-3-yl-1H-pyrazol-5-amine (200 mg, 0.93 mmol) in DCM (30 mL) was added a saturated aq. NaHCO₃ solution (20 mL). The heterogeneous mixture was stirred vigorously at 0° C., treated with diphosgene (0.50 mL, 3.7 mmol) in a single portion, and then warmed to room temperature for 1 h. The organic layer was separated, dried, and evaporated in vacuo to give a brown oil, which was triturated from hexane (2.0 mL) and filtered. The filtrate was concentrated in vacuo to give 3-(3-tert-butyl-5-isocyanato-1H-pyrazol-1-yl)pyridine 1.2 as a light brown oil (Yield: 40%, 90 mg).

### Step 2. Synthesis of Compound 1.4: 1-(3-(tert-butyl)-1-(pyridin-3-yl)-1H-pyrazol-5-yl)-3-(2-fluoro-4-((3-oxo-3,4-dihydropyrido[2,3-b]pyrazin-8-yl)oxy)phenyl)urea (Compound (I))

According to Scheme 1 Step 2: To a mixture of 8-(4-amino-3-fluorophenoxy)pyrido[2,3-b]pyrazin-3(4H)-one 8 (100 mg, 0.37 mmol) in DMF (3 mL) a 5-isocyanato-1-phenyl-1H-pyrazole 1.2 (58 mg, 0.37 mmol) was added, reaction mixture was stirred at 80° C. for 12 h. The reaction mixture was cooled and diluted with water. The precipitate was filtrated, washed with ether and dried. The solid was subjected to column chromatography on silica gel eluting with dichloromethane/ethyl acetate (0→100%) to afford the compound (I) (Yield: 21%, 40 mg). MS m/z (ES) [M+H]⁺: 515.30

### Synthesis of Compound (m): 1-(3-(tert-butyl)-1-(pyrimidin-2-yl)-1H-pyrazol-5-yl)-3-(2-fluoro-4-((3-oxo-3,4-dihydropyrido[2,3-b]pyrazin-8-yl)oxy)phenyl)urea

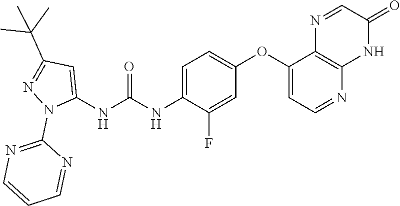 [SMILES: CC(C)(C)c1cc(NC(=O)Nc2ccc(Oc3ccnc4[nH]c(=O)cnc34)cc2F)n(-c2ncccn2)n1]

### Step 1. Synthesis of Compound 1.5

According to Scheme 2 Step 1: Compound 3 (2.97 g, 11.1 mmol, 1.15 eq) was dissolved in MeCN (20 mL) and 3-(tert-butyl)-1-(pyrimidin-2-yl)-1H-pyrazol-5-amine 1.1 (2.1 g, 9.66 mmol, 1 eq) was added to the solution. The reaction mixture was stirred at ambient temperature for 12 h, then the reaction mixture was evaporated under reduced pressure. The residue was purified by column chromatography silica gel (DCM) (Yield: 72%, 3.4 g).

### Step 2. Synthesis of Compound 1.6

According to Scheme 2 Step 2: Et₃N.3HF (11.4 mL, 70.1 mmol, 10 eq) was added to the solution of compound 1.5 (3.4 g, 7 mmol, 1 eq) in THF (30 mL) and the reaction mixture was stirred at ambient temperature for 12 h. The solvent was removed under reduced pressure, water was added to the residue, formed precipitate was filtered off, washed with water and dried to give product 1.6. (Yield: 85%, 2.2 g).

### Step 3. Synthesis of Compound 1.7

According to Scheme 2 Step 3: Compound 1.6 (2.2 g, 5.94 mmol, 1 eq) was added to the suspension of NaH (60%, 270 mg, 6.83 mmol, 1.15 eq) in DMSO (10 mL) and the reaction mixture was stirred at ambient temperature for 30 min. Then 4-chloro-3-nitropyridin-2-amine (1.08 g, 6.24 mmol, 1.05 eq) was added and the mixture was stirred at 60° C. for 12 h. Then the mixture was cooled down, water (50 mL) was added, the product was extracted with EtOAc (3×50 mL), the organic layers were dried under Na₂SO₄ and evaporated under reduced pressure. The residue was purified residue was purified by column chromatography on silica gel (DCM→EtOAc) (Yield: 20%, 600 mg).

### Step 4. Synthesis of Compound 1.8

According to Scheme 2 Step 4: A mixture of compound 1.7 (600 mg, 1.2 mmol, 1 eq) and 10% Pd/C (10% by weight, 60 mg) in MeOH (10 mL) was stirred at room temperature under flow of H₂ overnight. Then Pd/C was filtered, the solvent was evaporated under reduced pressure. The residue was used directly to the next step without additional purification. (Yield: 88%, 500 mg).

### Step 5. Synthesis of Compound 1.9: 1-(3-(tert-butyl)-1-(pyrimidin-2-yl)-1H-pyrazol-5-yl)-3-(2-fluoro-4-((3-oxo-3,4-dihydropyrido[2,3-b]pyrazin-8-yl)oxy)phenyl)urea (Compound (m))

According to Scheme 2 Step 5: A mixture of compound 1.8 (500 mg, 1.05 mmol, 1 eq) and ethyl oxoacetate (50% in toluene, 0.43 mL, 2.1 mmol, 2 eq) in EtOH (10 mL) was stirred at ambient temperature 12 h. The solvents were evaporated under reduced pressure, the residue was purified by column chromatography (DCM→DCM/MeOH 98:2→DCM/MeOH/NH₃ 98:2:0.1) to afford compound (m) (Yield: 1.1%, 6 mg). MS m/z (ES) [M+H]⁺: 516.40

### Synthesis of Compound (n): 1-(3-(tert-butyl)-1-(quinolin-6-yl)-1H-pyrazol-5-yl)-3-(2-fluoro-4-((3-oxo-3,4-dihydropyrido[2,3-b]pyrazin-8-yl)oxy)phenyl)urea

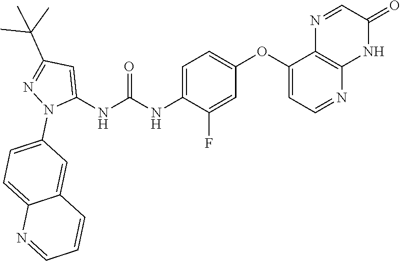 [SMILES: CC(C)(C)c1cc(NC(=O)Nc2ccc(Oc3ccnc4[nH]c(=O)cnc34)cc2F)n(-c2ccc3ncccc3c2)n1]

### Synthesis of Compound 1.4: 1-(3-(tert-butyl)-1-(quinolin-6-yl)-1H-pyrazol-5-yl)-3-(2-fluoro-4-((3-oxo-3,4-dihydropyrido[2,3-b]pyrazin-8-yl)oxy)phenyl)urea (Compound (n))

According to Scheme 4 Step 2: Mixture of compound 98 (200 mg, 0.45 mmol) and compound 8 (132 mg, 0.48 mmol) in anhydrous DMSO (2 mL) was stirred at 100° C. for 2 h. The reaction mixture was cooled and diluted with water and EtOAc. The precipitate was filtered off and collected. Organic layer separated, dried, combined with precipitate and evaporated. Residue dissolved in DCM/THF 4:1 and subjected to column chromatography (eluent DCM\THF 4:1) to afford 12 mg of the compound (n) (Yield: 4.4%). MS m/z (ES) [M+H]⁺: 565.40

### Synthesis of Compound (p): 1-(3-(tert-butyl)-1-phenyl-1H-1,2,4-triazol-5-yl)-3-(2-fluoro-4-((3-oxo-3,4-dihydropyrido[2,3-b]pyrazin-8-yl)oxy)phenyl)urea

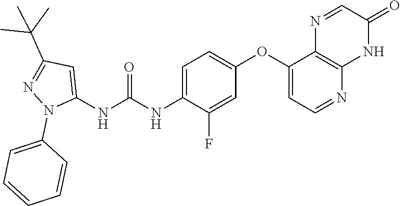 [SMILES: CC(C)(C)c1cc(NC(=O)Nc2ccc(Oc3ccnc4[nH]c(=O)cnc34)cc2F)n(-c2ccccc2)n1]

### Synthesis of Compound 1.4: 1-(3-(tert-butyl)-1-phenyl-1H-1,2,4-triazol-5-yl)-3-(2-fluoro-4-((3-oxo-3,4-dihydropyrido[2,3-b]pyrazin-8-yl)oxy)phenyl)urea (Compound (p))

According to Scheme 1 Step 2: Mixture of isocyanate 34 (130 mg, 0.53 mmol) and compound 8 (60 mg, 0.22 mmol) in anhydrous DMSO (2 mL) was stirred at 60° C. for 12 h. The reaction mixture was cooled and diluted with water and EtOAc. The precipitate was filtered off and discarded. Organic layer collected and product started to precipitate. This solid was filtered off washed with EtOAc and dried to afford the compound (p) (Yield: 10.6%, 12 mg). MS m/z (ES) [M+H]⁺: 515.30

### Synthesis of Compound (q): 1-(3-(tert-butyl)isothiazol-5-yl)-3-(2-fluoro-4-((3-oxo-3,4-dihydropyrido[2,3-b]pyrazin-8-yl)oxy)phenyl)urea

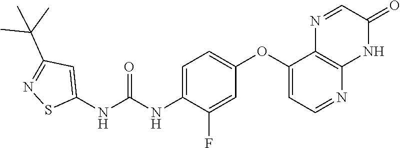 [SMILES: CC(C)(C)c1cc(NC(=O)Nc2ccc(Oc3ccnc4[nH]c(=O)cnc34)cc2F)sn1]

### Step 1. Synthesis of Compound 1.11

According to Scheme 4 Step 1: To a solution of 3-(tert-butyl)isothiazol-5-amine 1.1 (313 mg, 2 mmol) and 0.3 mL of pyridine in 10 mL of THF at 0° C. was added a solution of phenyl chloroformate (626 mg, 4 mmol) in THF. The mixture was stirred for 2 h at room temperature, treated with water, extracted with ethyl acetate, dried with sodium sulfate, and evaporated on a rotor. The residue was chromatographed, eluent ethyl acetate:hexane (1:4), to obtain the corresponding carbamate 1.11 (Yield: 65%, 450 mg).

### Step 2. Synthesis of Compound 1.4: 1-(3-(tert-butyl)isothiazol-5-yl)-3-(2-fluoro-4-((3-oxo-3,4-dihydropyrido[2,3-b]pyrazin-8-yl)oxy)phenyl)urea (Compound (q))

According to Scheme 4 Step 2: A mixture of compound 1.11 (176 mg, 0.64 mmol), compound 8 (174 mg, 0.64 mmol) and TEA (0.64 mmol) in 1 mL of dioxane was stirred for 12 h at 90° C. Cooled, poured into water, the precipitate was filtered off, washed with water, dried and further purified by HPLC to afford compound (q) (Yield: 5%, 14.5 mg). MS m/z (ES) [M+H]⁺: 455.50

### Synthesis of Compound (r): 1-(5-(tert-butyl)-1,3,4-thiadiazol-2-yl)-3-(2-fluoro-4-((3-oxo-3,4-dihydropyrido[2,3-b]pyrazin-8-yl)oxy)phenyl)urea

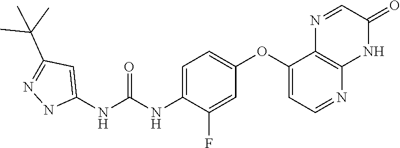 [SMILES: CC(C)(C)c1cc(NC(=O)Nc2ccc(Oc3ccnc4[nH]c(=O)cnc34)cc2F)[nH]n1]

### Synthesis of Compound 1.4: 1-(5-(tert-butyl)-1,3,4-thiadiazol-2-yl)-3-(2-fluoro-4-((3-oxo-3,4-dihydropyrido[2,3-b]pyrazin-8-yl)oxy)phenyl)urea (Compound (r))

One pot Scheme 1 following synthesis: To a mixture of 5-(tert-butyl)-1,3,4-thiadiazol-2-amine 104 (70 mg, 0.64 mmol), compound 8 (174 mg, 0.64 mmol) and TEA (0.64 mmol) in 1 mL of dioxane at 0° C., a solution of triphosgene (70 mg, 0.23 mmol) in 0.5 mL THF was added and stirred for 12 h at room temperature. The mixture was poured into water, the precipitate was filtered off, washed with water, dried and further purified by HPLC to afford compound (r) (Yield: 6%, 17.5 mg). MS m/z (ES) [M+H]⁺: 456.40

### Synthesis of Compound (s): 1-(3-(tert-butyl)-1-phenyl-1H-pyrazol-5-yl)-3-(4-((3-oxo-3,4-dihydropyrido[2,3-b]pyrazin-8-yl)oxy)phenyl)urea

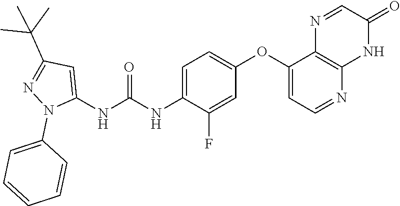 [SMILES: CC(C)(C)c1cc(NC(=O)Nc2ccc(Oc3ccnc4[nH]c(=O)cnc34)cc2F)n(-c2ccccc2)n1]

### Step 1. Synthesis of Compound 1.5

According to Scheme 2 Step 1: Compound 11 (4 g, 16 mmol, 1.15 eq) was dissolved in MeCN (20 mL) and 3-(tert-butyl)-1-phenyl-1H-pyrazol-5-amine 53 (3.0 g, 13.9 mmol, 1 eq) was added to the solution. The reaction mixture was stirred at ambient temperature for 12 h, then the reaction mixture was evaporated under reduced pressure. The residue was purified by column chromatography silica gel (DCM) to obtain compound 1.5 (Yield: 43%, 2.8 g).

### Step 2. Synthesis of Compound 1.6

According to Scheme 2 Step 2: Et₃N.3HF (9.8 mL, 60 mmol, 10 eq) was added to the solution of compound 1.5 (2.8 g, 6 mmol, 1 eq) in THF (20 mL) and the reaction mixture was stirred at ambient temperature for 12 h. The solvent was removed under reduced pressure, water was added to the residue, formed precipitate was filtered off, washed with water and dried to give product 1.6 (Yield: 57%, 1.2 g).

### Step 3. Synthesis of Compound 1.7

According to Scheme 2 Step 3: Compound 1.6 (1.2 g, 3.4 mmol, 1 eq) was added to the suspension of NaH (60%, 0.16 g, 3.91 mmol, 1.15 eq) in DMSO (10 mL) and the reaction mixture was stirred at ambient temperature for 30 min. Then 4-chloro-3-nitropyridin-2-amine (0.62 g, 3.57 mmol, 1.05 eq) was added and the mixture was stirred at 60° C. overnight. Then the mixture was cooled down, water (50 mL) was added, the product was extracted with EtOAc (3×50 mL), the organic layers were dried under Na₂SO₄ and evaporated under reduced pressure. The residue was purified residue was purified by column chromatography on silica gel (DCM→EtOAc) to get compound 1.7 (Yield: 36%, 600 mg).

### Step 4. Synthesis of Compound 1.8

According to Scheme 2 Step 4: A mixture of compound 1.7 (600 mg, 1.2 mmol, 1 eq) and 10% Pd/C (10% by weight, 0.06 g) in MeOH (10 mL) was stirred at room temperature under flow of H₂ for 12 h. Then Pd/C was filtered, the solvent was evaporated under reduced pressure. The residue 1.8 was used directly to the next step without additional purification (Yield: 89%, 500 mg).

### Step 5. Synthesis of Compound 1.9: 1-(3-(tert-butyl)-1-phenyl-1H-pyrazol-5-yl)-3-(4-((3-oxo-3,4-dihydropyrido[2,3-b]pyrazin-8-yl)oxy)phenyl)urea (Compound (s))

According to Scheme 2 Step 5: A mixture of compound 1.8 (500 mg, 1.09 mmol, 1 eq) and ethyl oxoacetate (50% in toluene, 0.33 mL, 1.63 mmol, 1.5 eq) in EtOH (10 mL) was stirred at ambient temperature for 12 h. The solvents were evaporated under reduced pressure, the residue was purified by column chromatography (DCM→DCM/MeOH 98:2→DCM/MeOH/NH₃ 98:2:0.1) to afford compound (s) (Yield: 3.7%, 20 mg). MS m/z (ES) [M+H]⁺: 496.20

### Synthesis of Compound (t): 1-(3-(tert-butyl)-1-phenyl-1H-pyrazol-5-yl)-3-(6-((3-oxo-3,4-dihydropyrido[2,3-b]pyrazin-8-yl)oxy)pyridin-3-yl)urea

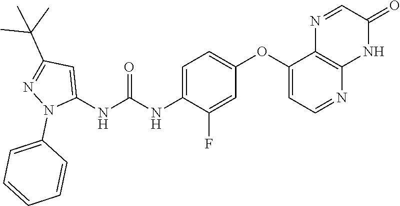 [SMILES: CC(C)(C)c1cc(NC(=O)Nc2ccc(Oc3ccnc4[nH]c(=O)cnc34)cc2F)n(-c2ccccc2)n1]

### Synthesis of Compound 1.4: 1-(3-(tert-butyl)-1-phenyl-1H-pyrazol-5-yl)-3-(6-((3-oxo-3,4-dihydropyrido[2,3-b]pyrazin-8-yl)oxy)pyridin-3-yl)urea (Compound (t))

According to Scheme 4 Step 2: To residue 54 (234 mg, 0.7 mmol) was added DMSO (10 mL) and compound 50 (179 mg, 0.7 mmol). Reaction mixture was stirred at 80° C. 1 h. The solvent was evaporated under reduced pressure and the residue after evaporation was separated by HPLC yield compound (t) (Yield: 8%, 27.8 mg). MS m/z (ES) [M+H]⁺: 497.5

### Synthesis of Compound (u): 1-(3-(tert-butyl)-1-phenyl-1H-pyrazol-5-yl)-3-(5-((3-oxo-3,4-dihydropyrido[2,3-b]pyrazin-8-yl)oxy)pyridin-2-yl)urea

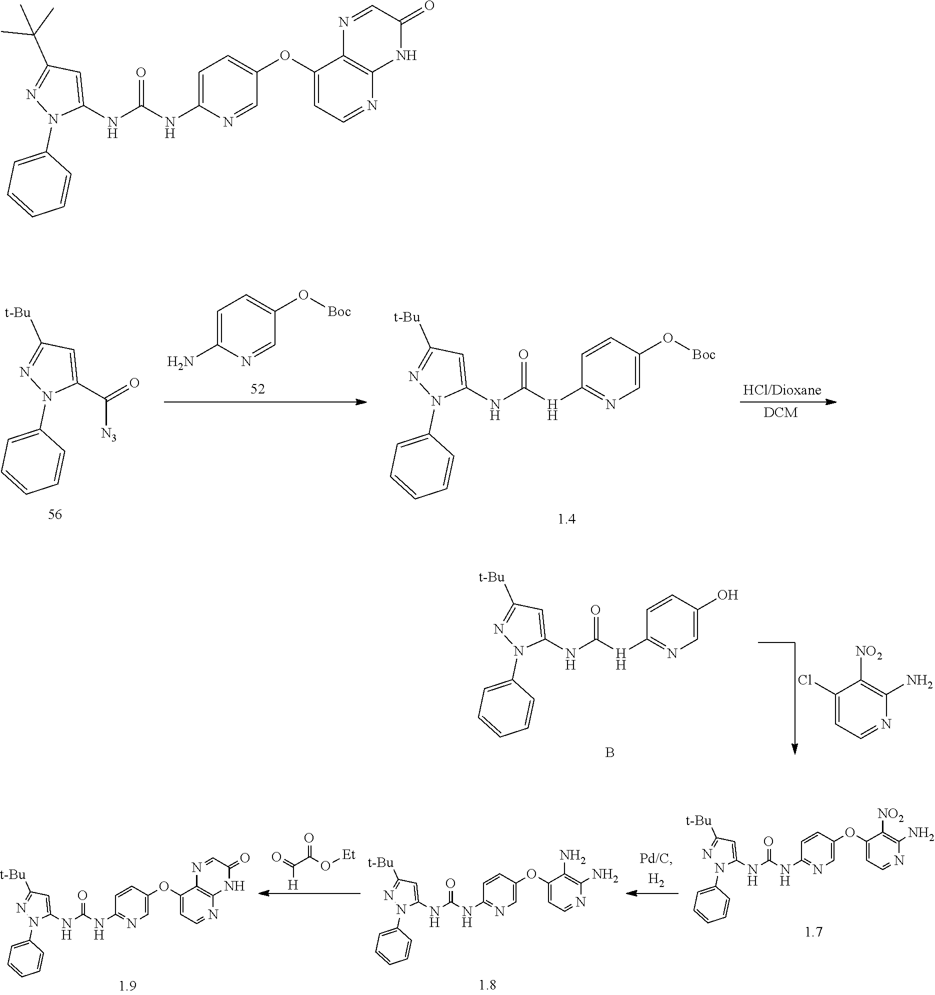

### Step 1. Synthesis of Compound 1.4

According to Scheme 5 Step 3: The crude acylazide 56 (1.2 g, 4.46 mmol) in DMSO (15 mL) was stirring for 1 h at 110° C. under inert atmosphere, reaction mixture was cooled to room temperature. The compound 52 (670 mg, 3.2 mmol) was added to the reaction mixture. After stirring for 12 h at 100° C. reaction mixture was cooled to room temperature, water (100 mL) was added and precipitate was filtered, washed with Et₂O (100 mL) and dried to afford 1.2 g of mixture of compound 1.4.

### Step 2. Synthesis of Compound B

1.2 g of the previous mixture was dissolved in 50 mL absolute DCM and 1 mL of 3M HCl in dioxane was added and stirred at ambient temperature for 60 min (control by TLC). The reaction mixture was diluted with ether (50 mL) and precipitate was filtered, washed with ether (100 mL). Then the precipitate was dissolved in mixture of DCM (50 mL) saturated aqueous NaHCO₃ (10 mL). Organic layer was separated, water-extracted with 2×20 mL of DCM. Combined organic layer was dried, filtrated and evaporated to dryness to afford 520 mg (Yield: 55%) of pure compound B.

### Step 3. Synthesis of Compound 1.7

According to Scheme 2 Step 3: NaH (60% w/w, 62 mg, 1.55 mmol) was added to a solution of the compound B (520 mg, 1.47 mmol) in absolute DMF (30 mL) and stirred at ambient temperature for 30 min under inert atmosphere. 4-Chloro-3-nitropyridin-2-amine (260 mg, 1.47 mmol) and the reaction mixture was heated at 80° C. for 30 min (control by TLC). The reaction mixture was cooled, diluted with water (150 mL) and formed precipitate was filtered off, washed with water, dried to afford pure compound 1.7 (Yield: 81%, 580 mg), which used in the next step without additional purification.

### Step 4. Synthesis of Compound 1.8

According to Scheme 2 Step 4: 10% Pd/C (300 mg) were added to a solution of compound 1.7 (580 mg, 1.19 mmol) in mixture EtOH/THF (100 mL, 2:1). The reaction mixture was hydrogenated at 20 atm. H₂ at 40° C. for 1 h. Then the catalyst was filtered off through Celite. The filtrate was evaporated under reduced pressure to afford of compound 1.8 (Yield: 89%, 480 mg), which used in the next step without additional purification.

### Step 5. Synthesis of Compound 1.9: 1-(3-(tert-butyl)-1-phenyl-1H-pyrazol-5-yl)-3-(5-((3-oxo-3,4-dihydropyrido[2,3-b]pyrazin-8-yl)oxy)pyridin-2-yl)urea (Compound (u))

According to Scheme 2 Step 5: The compound 1.8 (480 mg, 1.05 mmol) was dissolved in 50 mL of dry ethanol; 1.0 mL (5 mmol) of a 50% ethyl glyoxalate solution in toluene was added and the solution was stirred 36 h at room temperature under argon atmosphere. The solvent was partially evaporated and formed precipitate was filtered off. The filtrate containing regioisomer of 1.9 was discarded. The precipitate was purified by column chromatography on silica gel with THF as eluent, to afford (u) (Yield: 10%, 50 mg). MS m/z (ES) [M+H]⁺: 497.3

### Synthesis of Compound (v): 1-(3-(tert-butyl)-1-phenyl-1H-pyrazol-5-yl)-3-(4-((3-oxo-3,4-dihydropyrido[2,3-b]pyrazin-8-yl)oxy)-2-(trifluoromethyl)phenyl)urea

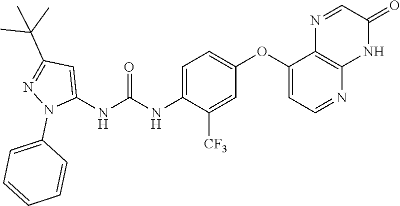 [SMILES: CC(C)(C)c1cc(NC(=O)Nc2ccc(Oc3ccnc4[nH]c(=O)cnc34)cc2C(F)(F)F)n(-c2ccccc2)n1]

### Step 1. Synthesis of Compound 1.5

According to Scheme 2 Step 1: The residue containing 59 was dissolved in DMSO (25 mL), and 3-(tert-butyl)-1-phenyl-1H-pyrazol-5-amine 53 (1.112 g, 5.15 mmol) was added to this solution. The reaction mass was stirred at 60° C. for 12 h, the reaction mass was poured in water (200 mL), the product was extracted with CH₂Cl₂ (2×30 mL), the organic layers were dried under Na₂SO₄, the solvents were removed under reduced pressure. The residue was purified by column chromatography, using hexane, hexane/Et₂O 3:1 as eluent, to provide product 1.5 (Yield: 27.1%, 743 mg).

### Step 2. Synthesis of Compound 1.6

According to Scheme 2 Step 2: A solution of 1.5 (743 mg, 1.39 mmol) and HCl (3M solution in dioxane, 2 mL) in methanol (20 mL) was stirred at room temperature for 12 h. The solvents were removed under reduced pressure, the residue was washed with CH₂Cl₂ and Et₂O to provide 1.6 (Yield: 99%, 580 mg).

### Step 3. Synthesis of Compound 1.7

According to Scheme 2 Step 3: Under Argon, NaH (60%, 0.055 g, 1.38 mmol) was added to a solution of 1.6 (580 mg, 1.38 mmol) in DMF (10 mL), the reaction mass was stirred at room temperature for 2 h. Then 4-chloro-3-nitropyridin-2-amine (280 mg, 1.62 mmol) was added, the mixture was stirred at room temperature for 12 h, then water (50 mL) was added, the product was extracted with CH₂Cl₂ (2×20 mL), the organic layers were dried under Na₂SO₄, the solvents were removed under reduced pressure. The residue was purified with twice crystallization from CH₂Cl₂ to provide the product 1.7 (Yield: 74%, 460 mg).

### Step 4. Synthesis of Compound 1.8

According to Scheme 2 Step 4: A mixture of 1.7 (460 mg, 0.828 mmol) and Pd/C (10%, 50 mg) in EtOH (20 mL) was stirred at room temperature under flow of H₂ for 12 h. Then Pd/C was filtered, the solvent from the mother liquor was removed under reduced pressure. The residue was purified by column chromatography, using CH₂Cl₂ and 2% MeOH in CH₂Cl₂ as eluent, to provide the product (Yield: 54%, 235 mg).

### Step 5. Synthesis of Compound 1.9: 1-(3-(tert-butyl)-1-phenyl-1H-pyrazol-5-yl)-3-(4-((3-oxo-3,4-dihydropyrido[2,3-b]pyrazin-8-yl)oxy)-2-(trifluoromethyl)phenyl)urea (Compound (v))

According to Scheme 2 Step 5: A mixture of 1.8 (235 mg, 0.447 mmol) and ethyl oxoacetate (137 mg of 50% in toluene, 0.671 mmol) in EtOH (10 mL) was stirred at room temperature for 12 h. The solvents were removed under reduced pressure, the residue was purified by column chromatography, using CH₂Cl₂ and 2% MeOH in CH₂Cl₂ as eluent, to provide (v) (Yield: 15.9%, 40 mg). MS m/z (ES) [M+H]⁺: 564.4

### Synthesis of Compound (w): 1-(3-(tert-butyl)-1-phenyl-1H-pyrazol-5-yl)-3-(2-fluoro-4-((2-methyl-4-oxo-3,4-dihydropyrido[3,2-d]pyrimidin-8-yl)oxy)phenyl)urea

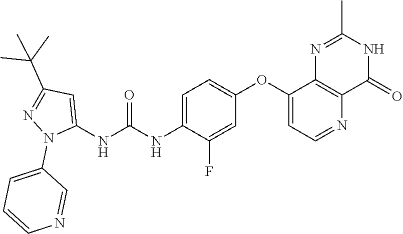 [SMILES: Cc1nc2c(Oc3ccc(NC(=O)Nc4cc(C(C)(C)C)nn4-c4cccnc4)c(F)c3)ccnc2c(=O)[nH]1]

### Synthesis of Compound 1.4: 1-(3-(tert-butyl)-1-phenyl-1H-pyrazol-5-yl)-3-(2-fluoro-4-((2-methyl-4-oxo-3,4-dihydropyrido[3,2-d]pyrimidin-8-yl)oxy)phenyl)urea (Compound (w))

According to Scheme 4 Step 2: To a solution of compound 80 (200 mg, 0.70 mmol) in anhydrous DMSO (2 mL) was a compound 54 (234 mg, 0.70 mmol) and reaction mixture was stirred at 100° C. for 12 h. After cooling mixture quenched with water and EtOAc. Precipitate filtered, washed with water, then EtOAc and dried. Then redissolved in THF and filtered through celite pad. Filtrate evaporated to afford pure compound (w) (Yield: 27.6%, 102 mg) as white crystalline solid. MS m/z (ES) [M+H]⁺: 528.6

### Synthesis of Compound (x): 1-(2-fluoro-4-((3-oxo-3,4-dihydropyrido[2,3-b]pyrazin-8-yl)oxy)phenyl)-3-(2-phenyl-2,4,5,6-tetrahydrocyclopenta[c]pyrazol-3-yl)urea

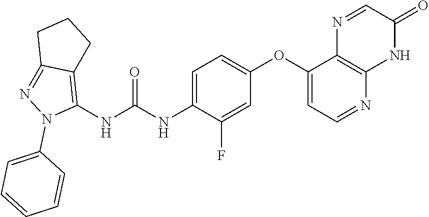 [SMILES: O=C(Nc1ccc(Oc2ccnc3[nH]c(=O)cnc23)cc1F)Nc1c2c(nn1-c1ccccc1)CCC2]

### Step 1. Synthesis of Compound 1.2

According to Scheme 1 Step 1: To a mixture of 2-phenyl-2,4,5,6-tetrahydrocyclopenta[c]pyrazol-3-amine 1.1 (200 mg, 1.0 mmol) and DIPEA (0.5 mL, 3.0 mmol) in dichloromethane (5 mL) triphosgene (290 mg, 1.0 mmol) was added at 0° C. and the reaction mixture was stirred 16 h. Then reaction mixture was evaporated to dryness, the residue was dissolved in THF and precipitate was removed by filtration and the solution was evaporated to dryness. The product 1.2 was used in next synthesis without purification.

### Step 2. Synthesis of Compound 1.4: 1-(2-fluoro-4-((3-oxo-3,4-dihydropyrido[2,3-b]pyrazin-8-yl)oxy)phenyl)-3-(2-phenyl-2,4,5,6-tetrahydrocyclopenta[c]pyrazol-3-yl)urea (Compound (x))

According to Scheme 1 Step 2: To a solution of compound 1.2 (125 mg, 0.54 mmol) and compound 8 (100 mg, 0.36 mmol) in DMSO (2 mL) was stirred at 60° C. for 12 h. The reaction mixture was cooled and diluted with water. The precipitate was filtrated, washed with ether (100 mL) and dried. The solid after evaporation was subjected to column chromatography on silica gel eluting with dichloromethane/ethyl acetate (0→100%) to afford the compound (x) (Yield: 14%, 25 mg). MS m/z (ES) [M+H]⁺: 498.50

### Synthesis of Compound (y): 1-(2-fluoro-4-((3-oxo-3,4-dihydropyrido[2,3-b]pyrazin-8-yl)oxy)phenyl)-3-(2-phenyl-5,6-dihydro-4H-pyrrolo[1,2-b]pyrazol-3-yl)urea

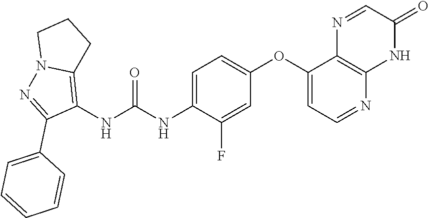 [SMILES: O=C(Nc1ccc(Oc2ccnc3[nH]c(=O)cnc23)cc1F)Nc1c(-c2ccccc2)nn2c1CCC2]

### Steps 1 & 2. Synthesis of Compound 1.14

According to Scheme 5 Steps 1 & 2: A solution of 89 (503 mg, 2.21 mmol) in SOCl₂ (20 mL) was stirred at boiling temperature for 2 h. The excess of SOCl₂ was removed under reduced pressure, the residue was dissolved in THF (20 mL), NaN₃ (287 mg, 4.42 mmol) was added, the mixture was stirred at room temperature for 1 h. Water (50 mL) was added, the product was extracted with CH₂Cl₂ (2×30 mL), the combined organic layers were dried under Na₂SO₄, the solvents were removed under reduced pressure to provide the product. (Yield: 82%, 459 mg).

### Steps 3. Synthesis of Compound 1.4: 1-(2-fluoro-4-((3-oxo-3,4-dihydropyrido[2,3-b]pyrazin-8-yl)oxy)phenyl)-3-(2-phenyl-5,6-dihydro-4H-pyrrolo[1,2-b]pyrazol-3-yl)urea (Compound (y))

According to Scheme 5 Step 3: A solution of 1.14 (459 mg, 1.81 mmol) in toluene (10 mL) was stirred at boiling temperature for 1.5 h. The solvent was removed under reduced pressure, the residue was dissolved in DMSO (10 mL), aniline 8 (287 mg, 4.42 mmol) was added, the mixture was stirred at 60° C. for 12 h. Water (50 mL) was added, the product was extracted with EtOAc (2×30 mL), the combined organic layers were dried under Na₂SO₄, the solvent was removed under reduced pressure, the residue was purified twice by crystallization from EtOAc and five times by column chromatography, using CH₂Cl₂ and 2% MeOH in CH₂Cl₂ as eluent, to provide the product (y) (Yield: 0.4%, 4 mg). MS m/z (ES) [M+H]⁺: 498.40

### Synthesis of Compound (z): 1-(5-(tert-butyl)-1,3,4-thiadiazol-2-yl)-3-(2-(methylthio)-4-((3-oxo-3,4-dihydropyrido[2,3-b]pyrazin-8-yl)oxy)phenyl)urea

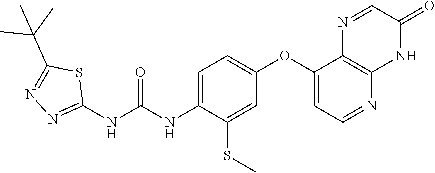 [SMILES: CSc1cc(Oc2ccnc3[nH]c(=O)cnc23)ccc1NC(=O)Nc1nnc(C(C)(C)C)s1]

### Synthesis of Compound 1.4: 1-(5-(tert-butyl)-1,3,4-thiadiazol-2-yl)-3-(2-(methylthio)-4-((3-oxo-3,4-dihydropyrido[2,3-b]pyrazin-8-yl)oxy)phenyl)urea (Compound (z))

According to Scheme 4 Step 2: To a solution of compound 111 (270 mg, 0.90 mmol, 1 eq) and compound 105 (300 mg, 0.90 mmol, 1 eq) in 4 mL of DMSO was added N,N-diisopropylethylamine (502 μL, 2.08 mmol, 3.2 eq). The mixture was stirred at 70° C. for 8 h. After cooling down, water was added and the mixture was extracted with ethyl acetate. The orange residue was triturated in dichloromethane and methanol and filtered. The powder was purified by preparative HPLC (C8 spheric column, 150×30 mm, gradient of CH₃CN (5 to 100%) in water+0.1% HCOOH). The desired fractions were evaporated to dryness to afford the expected compound (z) as white solid (Yield: 2%, 9.6 mg). MS m/z (ES) [M+H]⁺: 484.40

### Synthesis of Compound (aa): 4-(3-(3-(3-(tert-butyl)-1-phenyl-1H-pyrazol-5-yl)ureido)phenoxy)-N-methylpicolinamide

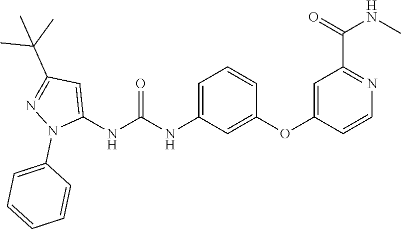 [SMILES: CNC(=O)c1cc(Oc2cccc(NC(=O)Nc3cc(C(C)(C)C)nn3-c3ccccc3)c2)ccn1]

### Synthesis of Compound 1.4: 4-(3-(3-(3-(tert-butyl)-1-phenyl-1H-pyrazol-5-yl)ureido)phenoxy)-N-methylpicolinamide (Compound (aa))

According to Scheme 1 Step 2: To a solution of compound 64 (197 mg, 0.81 mmol) and compound 54 (272 mg, 0.81 mmol) in DMSO (10 mL) was stirred at 60° C. 12 h. The reaction mixture was cooled and diluted with water. The precipitate was filtrated, washed with ether (100 mL) and dried. The solid after evaporation was subjected to column chromatography on silica gel eluting with dichloromethane/ethyl acetate (0→100%) to afford the compound (aa) (Yield: 3.7%, 15 mg). MS m/z (ES) [M+H]⁺: 485.50

### Synthesis of Compound (ab): 5-(3-(3-(3-(tert-butyl)-1-phenyl-1H-pyrazol-5-yl)ureido)phenoxy)-N-methylpicolinamide

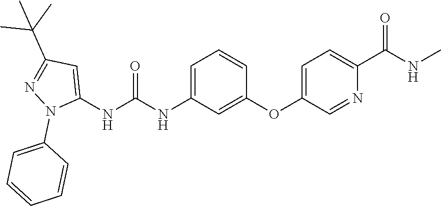 [SMILES: CNC(=O)c1ccc(Oc2cccc(NC(=O)Nc3cc(C(C)(C)C)nn3-c3ccccc3)c2)cn1]

### Synthesis of Compound 1.4: 5-(3-(3-(3-(tert-butyl)-1-phenyl-1H-pyrazol-5-yl)ureido)phenoxy)-N-methylpicolinamide (Compound (ab))

According to Scheme 1 Step 2: To a solution of compound 69 (197 mg, 0.81 mmol) and compound 54 (272 mg, 0.81 mmol) in DMSO (10 mL) was stirred at 60° C. 12 h. The reaction mixture was cooled and diluted with water. The precipitate was filtrated, washed with ether (100 mL) and dried. The solid after evaporation was subjected to column chromatography on silica gel eluting with dichloromethane/ethyl acetate (0→100%) to afford the compound (ab) (Yield: 2.5%, 10 mg). MS m/z (ES) [M+H]⁺: 485.50

### Synthesis of Compound (ac): 1-(3-(tert-butyl)-1-phenyl-1H-pyrazol-5-yl)-3-(2-fluoro-4-phenoxyphenyl)urea

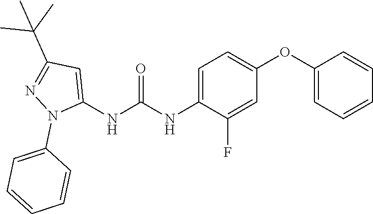 [SMILES: CC(C)(C)c1cc(NC(=O)Nc2ccc(Oc3ccccc3)cc2F)n(-c2ccccc2)n1]

### Step 1. Synthesis of Compound 1.10

According to Scheme 3 Step 1: Under Argon, triphosgene (143 mg, 0.483 mmol) was added portions to a solution of 71 (98 mg, 0.483 mmol) and Et₃N (0.08 mL, 0.58 mmol) in CH₂Cl₂ (5 mL) at −20° C., the reaction mass was stirred at room temperature for 1 h. Then the solvent was removed under reduced pressure, THF (5 mL) was added to the residue, the formed precipitate was filtered. The crude residue was used in the next step without further purification.

### Step 2. Synthesis of Compound 1.4: 1-(3-(tert-butyl)-1-phenyl-1H-pyrazol-5-yl)-3-(2-fluoro-4-phenoxyphenyl)urea (Compound (ac))

According to Scheme 3 Step 2: 3-(tert-butyl)-1-phenyl-1H-pyrazol-5-amine 53 (108 mg, 0.483 mmol) was added to the mother liquor. The reaction mass was stirred at room temperature for 12 h, the solvent was removed under reduced pressure, the residue was purified by column chromatography, using hexane and hexane/Et₂O 3:1 as eluent. The obtained impure product (73 mg) was purified with crystallization from H₂O/CH₃CN 1:1 to provide the pure product (ac) (Yield: 22.3%, 48 mg). MS m/z (ES) [M+H]⁺: 445.5

### Synthesis of Compound (ad): 1-(3-(tert-butyl)-1-phenyl-1H-pyrazol-5-yl)-3-(2-fluoro-4-(pyridin-4-yloxy)phenyl)urea

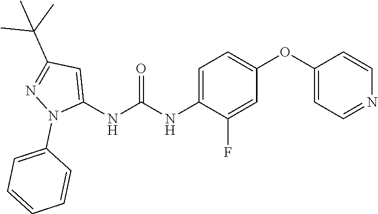 [SMILES: CC(C)(C)c1cc(NC(=O)Nc2ccc(Oc3ccncc3)cc2F)n(-c2ccccc2)n1]

### Synthesis of Compound 1.4: 1-(3-(tert-butyl)-1-phenyl-1H-pyrazol-5-yl)-3-(2-fluoro-4-(184pyridine-4-yloxy)phenyl)urea (Compound (ad))

According to Scheme 5 Step 3: Under Argon a solution of 56 (458 mg, 1.7 mmol) in toluene (10 mL) was stirred at 115° C. for 1 h, then the solvent was removed under reduced pressure, the residue was dissolved in DMSO (10 mL), and aniline 72 (170 mg, 0.833 mmol) was added to this solution. The reaction mass was stirred at 60° C. for 12 h. The mixture was poured in water (50 mL), the product was extracted with EtOAc (2×20 mL), the organic layers were dried under Na₂SO₄, the solvent was removed under reduced pressure. The residue was purified twice by column chromatography, using CH₂Cl₂ and 2% MeOH in CH₂Cl₂ as eluent. The obtained impure product was purified by crystallization from EtOH to provide the product (ad) (Yield: 6.7%, 25 mg). MS m/z (ES) [M+H]⁺: 446.2

### Synthesis of Compound (ae): N-(4-(4-(3-(3-(tert-butyl)-1-phenyl-1H-pyrazol-5-yl)ureido)-3-fluorophenoxy)pyridin-2-yl)acetamide

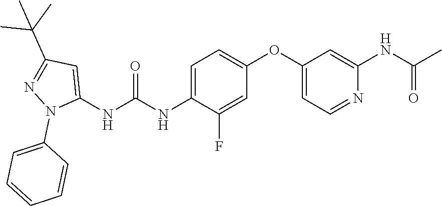 [SMILES: CC(=O)Nc1cc(Oc2ccc(NC(=O)Nc3cc(C(C)(C)C)nn3-c3ccccc3)c(F)c2)ccn1]

### Synthesis of Compound 1.4: N-(4-(4-(3-(3-(tert-butyl)-1-phenyl-1H-pyrazol-5-yl)ureido)-3-fluorophenoxy)pyridin-2-yl)acetamide (Compound (ae))

According to Scheme 5 Step 3: A solution of compound 56 (150 mg, 0.55 mmol) in DMSO (2 mL) was stirring for 2 h at 120° C., reaction mixture cooling to room temperature. Compound 75 (110 mg, 0.4 mmol) was added to a reaction mixture. After stirring for 12 h at 100° C., reaction mixture cooling to room temperature, water (25 mL) was added to the reaction mixture, and the resulting mixture was diluted with Et₂O (50 mL). The crude product was filtered and purified by crystallization from acetonitrile to obtain compound (ae) (Yield: 52%, 110 mg). MS m/z (ES) [M+H]⁺: 503.30

### Synthesis of Compound (af): 1-(3-(tert-butyl)-1-phenyl-1H-pyrazol-5-yl)-3-(2-fluoro-4-(4-fluorophenoxy)phenyl)urea

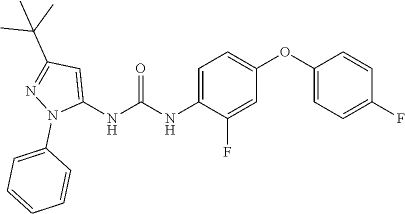 [SMILES: CC(C)(C)c1cc(NC(=O)Nc2ccc(Oc3ccc(F)cc3)cc2F)n(-c2ccccc2)n1]

### Step1. Synthesis of Compound 1.10

According to Scheme 3 Step 1: Under Aron, triphosgene (134 mg, 0.452 mmol) was added portionwise to a solution of 81 (100 mg, 0.452 mmol) and Et₃N (0.075 mL, 0.542 mmol) in CH₂Cl₂ (5 mL) at −20° C., the reaction mass was stirred at room temperature for 1 h. Then the solvent was removed under reduced pressure, THF (5 mL) was added to the residue, the formed precipitate was filtered.

### Step 2. Synthesis of Compound 1.4: 1-(3-(tert-butyl)-1-phenyl-1H-pyrazol-5-yl)-3-(2-fluoro-4-(4-fluorophenoxy)phenyl)urea (Compound (af))

According to Scheme 3 Step 2: 3-(tert-butyl)-1-phenyl-1H-pyrazol-5-amine 53 (97 mg, 0.452 mmol) was added to the mother liquor. The reaction mass was stirred at room temperature for 12 h, the solvent was removed under reduced pressure, the residue was purified twice by column chromatography, using hexane and hexane/Et₂O 3:1. The obtained impure product (120 mg) was purified by crystallization from H₂O/CH₃CN 1:1 to provide the pure product (af) (Yield: 27.7%, 58 mg). MS m/z (ES) [M+H]⁺: 463.40

### Synthesis of Compound (ag): 4-(4-(3-(3-(tert-butyl)-1-(quinolin-6-yl)-1H-pyrazol-5-yl) ureido)-3-(methylthio)phenoxy)-N-methylpicolinamide

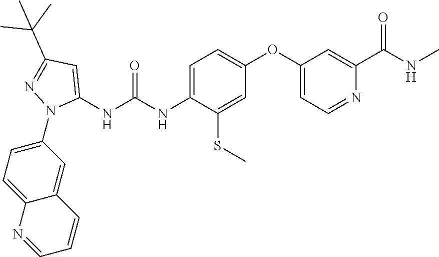 [SMILES: CNC(=O)c1cc(Oc2ccc(NC(=O)Nc3cc(C(C)(C)C)nn3-c3ccc4ncccc4c3)c(SC)c2)ccn1]

### Synthesis of Compound 1.4: 4-(4-(3-(3-(tert-butyl)-1-(quinolin-6-yl)-1H-pyrazol-5-yl)ureido)-3-(methylthio)phenoxy)-N-methylpicolinamide (Compound (ag))

According to Scheme 4 Step 2: To a solution of compound 98 (580 mg, 1.31 mmol, 1 eq) and compound 103 (380 mg, 1.31 mmol, 1 eq) in 6 mL of DMSO was added N, N-diisopropylethylamine (0.5 mL, 2.89 mmol, 2.2 eq). The mixture was stirred at 70° C. for 8 h. After cooling down, water was added. The aqueous layer was washed with ethyl acetate and evaporated to dryness. The residue was purified by flash chromatography on silica gel using cyclohexane/ethyl acetate then ethyl acetate/methanol to a afford the expected compound (ag) as white powder (Yield: 16%, 125 mg). MS m/z (ES) [M+H]⁺: 582.40

### Synthesis of Compound (ah): 1-(5-(tert-butyl)thiazol-2-yl)-3-(2-(methylthio)-4-((3-oxo-3,4-dihydropyrido[2,3-b]pyrazin-8-yl)oxy)phenyl)urea

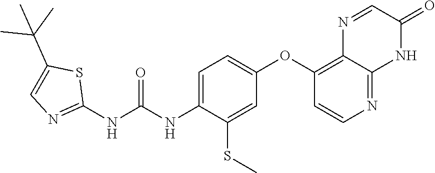 [SMILES: CSc1cc(Oc2ccnc3[nH]c(=O)cnc23)ccc1NC(=O)Nc1ncc(C(C)(C)C)s1]

### Synthesis of Compound 1.4: 1-(5-(tert-butyl)thiazol-2-yl)-3-(2-(methylthio)-4-((3-oxo-3,4-dihydropyrido[2,3-b]pyrazin-8-yl)oxy)phenyl)urea (Compound (ah))

According to Scheme 4 Step 2: In a sealed tube containing 8-(4-amino-3-(methylthio)phenoxy)pyrido[2,3-b]pyrazin-3(4H)-one, hydrochloride 111 (194 mg, 0.65 mmol, 1.0 eq), DIEA (6 eq) in DMSO (0.1M) was added 2,2,2-trichloroethyl N-(5-tert-butylthiazol-2-yl)carbamate 157 (261 mg, 0.65 mmol, 1.0 eq). The reaction mixture was stirred at 70° C. for 20 h then 100° C. during 6 h. The residue was purified by semi-preparative HPLC to afford expected compound as beige powder (Yield: 23%, 43 mg). MS m/z (ES) [M+H]⁺: 483.42

### Synthesis of Compound (ai): 4-(4-(3-(3-(tert-butyl)-1-phenyl-1H-pyrazol-5-yl)ureido)-3-(methylthio)phenoxy)-N-methylpicolinamide

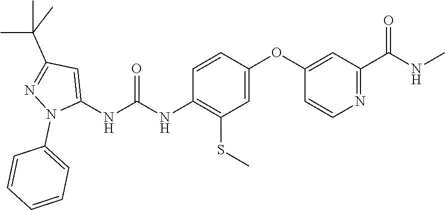 [SMILES: CNC(=O)c1cc(Oc2ccc(NC(=O)Nc3cc(C(C)(C)C)nn3-c3ccccc3)c(SC)c2)ccn1]

### Step2. Synthesis of Compound 1.4: 4-(4-(3-(3-(tert-butyl)-1-phenyl-1H-pyrazol-5-yl)ureido)-3-(methylthio)phenoxy)-N-methylpicolinamide (Compound (ai))

According to Scheme 4 Step 2: In a sealed tube containing 4-(4-amino-3-methylsulfanyl-phenoxy)-N-methyl-pyridine-2-carboxamide 103 (120 mg, 0.41 mmol), DIEA (0.40 mL, 2.48 mmol) in DMSO (0.1M) was added 2,2,2-trichloroethyl N-(5-tert-butyl-2-phenyl-pyrazol-3-yl)carbamate 158 (242 mg, 0.62 mmol). The reaction mixture was stirred at 70° C. for 24 h to afford after extraction, flash chromatography on silica gel (12 g, SiO₂, dichloromethane/AcOEt 100:0→0:100, 10 CV) to afford expected compound as a red foam (Yield: 18%, 40 mg). MS m/z (ES) [M+H]⁺: 531.42

### Synthesis of Compound (aj): 1-(3-(tert-butyl)-1-(4-methoxyphenyl)-1H-pyrazol-5-yl)-3-(2-(methylthio)-4-((3-oxo-3,4-dihydropyrido[2,3-b]pyrazin-8-yl)oxy)phenyl)urea

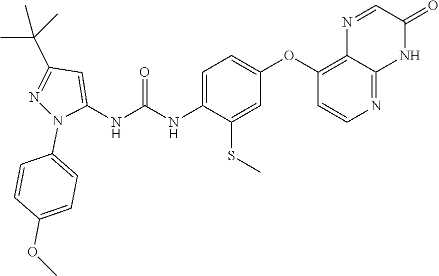 [SMILES: COc1ccc(-n2nc(C(C)(C)C)cc2NC(=O)Nc2ccc(Oc3ccnc4[nH]c(=O)cnc34)cc2SC)cc1]

### Step1. Synthesis of Compound 1.11

According to Scheme 4 Step 1: At 0° C., to a solution of N-[5-tert-butyl-2-(4-methoxyphenyl)pyrazol-3-yl]amine 309 (500 mg, 2.04 mmol, 1.0 eq) in dry THF (0.2M) was added dropwise dry pyridine (1.1 eq) followed by 2,2,2-trichloroethylchloroformate (0.31 mL, 2.24 mmol, 1.1 eq). The reaction mixture was stirred at room temperature for 18 h. Water was added, and the reaction mixture was extracted with ethyl acetate. The organic layers were combined, dried over magnesium sulfate, filtered and evaporated. The compound was obtained as brown gum (900 mg, quantitative yield) and was used without purification in the next step.

### Step2. Synthesis of Compound 1.4: 1-(3-(tert-butyl)-1-(4-methoxyphenyl)-1H-pyrazol-5-yl)-3-(2-(methylthio)-4-((3-oxo-3,4-dihydropyrido[2,3-b]pyrazin-8-yl)oxy)phenyl)urea (Compound (aj))

According to Scheme 4 Step 2: In a sealed tube containing 8-(4-amino-3-(methylthio)phenoxy)pyrido[2,3-b]pyrazin-3(4H)-one hydrochloride 111 (210 mg, 0.50 mmol, 1.0 eq), DIEA (6 eq) in DMSO (0.1M) was added 2,2,2-trichloroethyl N-[5-tert-butyl-2-(4-methoxyphenyl)pyrazol-3-yl]carbamate (210 mg, 0.50 mmol, 1.0 eq). The reaction mixture was stirred at 70° C. for 18 h The residue was purified by 2 flash chromatographies on silica gel (cyclohexane/ethyl acetate then ethyl acetate/methanol) and a semi-preparative HPLC to afford expected compound as white powder (Yield: 30%, 30 mg). MS m/z (ES) [M+H]⁺: 572.42

### Synthesis of Compound (ak): 1-(3-(tert-butyl)-1-phenyl-1H-pyrazol-5-yl)-3-(2-chloro-4-((3-oxo-3,4-dihydropyrido[2,3-b]pyrazin-8-yl)oxy)phenyl)urea

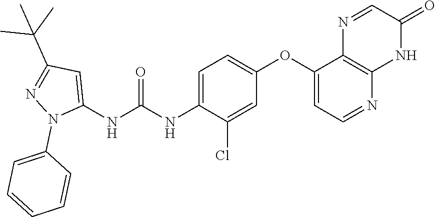 [SMILES: CC(C)(C)c1cc(NC(=O)Nc2ccc(Oc3ccnc4[nH]c(=O)cnc34)cc2Cl)n(-c2ccccc2)n1]

### Synthesis of Compound 1.4: 1-(3-(tert-butyl)-1-phenyl-1H-pyrazol-5-yl)-3-(2-chloro-4-((3-oxo-3,4-dihydropyrido[2,3-b]pyrazin-8-yl)oxy)phenyl)urea (Compound (ak))

According to Scheme 4 Step 2: In a sealed tube 8-(4-amino-3-chloro-phenoxy)-4H-pyrido[2,3-b]pyrazin-3-one hydrochloride 117 (250 mg, 0.87 mmol, 1.0 eq), DIEA (1.5 mL, 8.7 mmol, 10.0 eq) in DMSO (0.1M) was added 2,2,2,-trichloroethyl N-(5-tert-butyl-2-phenyl-pyrazol-3-yl)carbamate 158 (305 mg, 0.87 mmol, 1.0 eq). The mixture was stirred at 70° C. for 18 h. The expected compound was obtained after 2 flash chromatographies (cyclohexane/ethyl acetate then ethyl acetate/methanol) and semi-preparative HPLC (elution: water/CH₃CN) as rose orange solid (Yield: 8%, 39 mg). MS m/z (ES) [M+H]⁺: 530.42

### Synthesis of Compound (al): 1-(3-(tert-butyl)-1-phenyl-1H-pyrazol-5-yl)-3-(2-methyl-4-((3-oxo-3,4-dihydropyrido[2,3-b]pyrazin-8-yl)oxy)phenyl)urea

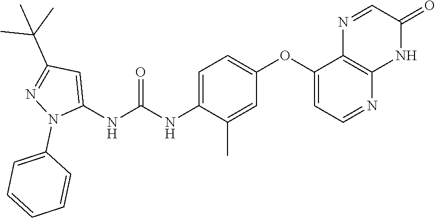 [SMILES: Cc1cc(Oc2ccnc3[nH]c(=O)cnc23)ccc1NC(=O)Nc1cc(C(C)(C)C)nn1-c1ccccc1]

### Synthesis of Compound 1.4: 1-(3-(tert-butyl)-1-phenyl-1H-pyrazol-5-yl)-3-(2-methyl-4-((3-oxo-3,4-dihydropyrido[2,3-b]pyrazin-8-yl)oxy)phenyl)urea (Compound (al))

According to Scheme 4 Step 2: In a sealed tube 8-(4-amino-3-methyl-phenoxy)-4H-pyrido[2,3-b]pyrazin-3-one hydrochloride 123 (209 mg, 0.78 mmol, 1.0 eq), DIEA (1.3 mL, 7.8 mmol, 10.0 eq) in DMSO (0.1M) was added 2,2,2,-trichloroethyl N-(5-tert-butyl-2-phenyl-pyrazol-3-yl)carbamate 158 (305 mg, 0.78 mmol, 1.0 eq). The mixture was stirred at 70° C. for 18 h. The expected compound was obtained after 2 flash chromatographies (cyclohexane/ethyl acetate then ethyl acetate/methanol) and semi-preparative HPLC (elution: water/CH₃CN) as white solid (Yield: 26%, 102 mg). MS m/z (ES) [M+H]⁺: 510.42

### Synthesis of Compound (am): 1-(3-(tert-butyl)-1-phenyl-1H-pyrazol-5-yl)-3-(2-(ethylthio)-4-((3-oxo-3,4-dihydropyrido[2,3-b]pyrazin-8-yl)oxy)phenyl)urea

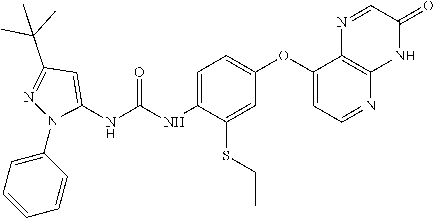 [SMILES: CCSc1cc(Oc2ccnc3[nH]c(=O)cnc23)ccc1NC(=O)Nc1cc(C(C)(C)C)nn1-c1ccccc1]

### Synthesis of Compound 1.4: 1-(3-(tert-butyl)-1-phenyl-1H-pyrazol-5-yl)-3-(2-(ethylthio)-4-((3-oxo-3,4-dihydropyrido[2,3-b]pyrazin-8-yl)oxy)phenyl)urea (Compound (am))

According to Scheme 4 Step 2: In a sealed tube 8-(4-amino-3-ethylsulfanyl-phenoxy)-4H-pyrido[2,3-b]pyrazin-3-one, hydrochloride 130 (180 mg, 0.51 mmol, 1.0 eq) in acetonitrile was added 2,2,2,-trichloroethyl N-(5-tert-butyl-2-phenyl-pyrazol-3-yl)carbamate 158 (200 mg, 0.51 mmol, 1.0 eq). The mixture was stirred at 70° C. for 18 h to afford after extraction, flash chromatography and semi-preparative HPLC (elution: water/CH₃CN), the title compound as white solid (Yield: 14%, 41 mg). MS m/z (ES) [M+H]⁺: 556.33

### Synthesis of Compound (an): 1-(3-(tert-butyl)-1-phenyl-1H-pyrazol-5-yl)-3-(2-(methylthio)-4-((7-oxo-5,6,7,8-tetrahydro-1,8-naphthyridin-4-yl)oxy)phenyl)urea

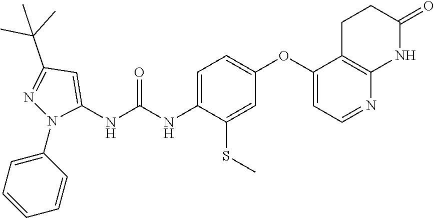 [SMILES: CSc1cc(Oc2ccnc3c2CCC(=O)N3)ccc1NC(=O)Nc1cc(C(C)(C)C)nn1-c1ccccc1]

### Synthesis of Compound 1.4: 1-(3-(tert-butyl)-1-phenyl-1H-pyrazol-5-yl)-3-(2-(methylthio)-4-((7-oxo-5,6,7,8-tetrahydro-1,8-naphthyridin-4-yl)oxy)phenyl)urea (Compound (an))

According to Scheme 4 Step 2: In a sealed tube containing 5-(4-amino-3-methylsulfanyl-phenoxy)-3,4-dihydro-1H-1,8-naphthyridin-2-one 132 (102 mg, 0.34 mmol), DIEA (0.35 mL, 2.03 mmol) in acetonitrile (0.1M) was added 2,2,2-trichloroethyl N-(5-tert-butyl-2-phenyl-pyrazol-3-yl)carbamate 158 (159 mg, 0.40 mmol). The reaction mixture was stirred at 70° C. for 18 h to furnish, after filtration and purification by semi-preparative HPLC (elution: water/CH₃CN) the title compound (Yield: 25%, 47 mg) as a pink powder. MS m/z (ES) [M+H]⁺: 543.25

¹H NMR (300 MHz, DMSO-d₆) δ 10.48 (s, 1H), 8.96 (s, 1H), 8.34 (s, 1H), 7.95 (d, J=5.7 Hz, 1H), 7.70 (d, J=8.8 Hz, 1H), 7.52-7.49 (m, 4H), 7.42-7.36 (m, 1H), 7.09 (d, J=2.6 Hz, 1H), 6.91 (dd, J=8.8, 2.7 Hz, 1H), 6.34 (s, 1H), 6.29 (d, J=5.8 Hz, 1H), 2.90 (t, J=7.3 Hz, 2H), 2.51 (t, J=7.3 Hz, 2H), 2.40 (s, 3H), 1.26 (s, 9H).

### Synthesis of Compound (ao): 1-(3-(tert-butyl)-1-(4-chlorophenyl)-1H-pyrazol-5-yl)-3-(2-(methylthio)-4-((3-oxo-3,4-dihydropyrido[2,3-b]pyrazin-8-yl)oxy)phenyl)urea

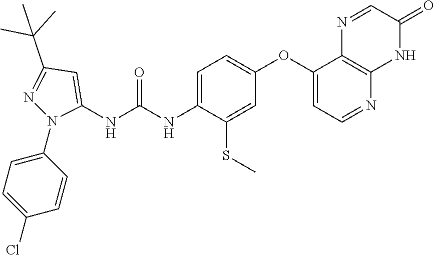 [SMILES: CSc1cc(Oc2ccnc3[nH]c(=O)cnc23)ccc1NC(=O)Nc1cc(C(C)(C)C)nn1-c1ccc(Cl)cc1]

### Step1. Synthesis of Compound 1.11

According to Scheme 4 Step 1: At 0° C., to a solution of N-[5-tert-butyl-2-(4-chlorophenyl)pyrazol-3-yl]amine (500 mg, 2.0 mmol, 1.0 eq)) in dry THF (0.2M) was added dropwise dry pyridine (1.1 eq) followed by 2,2,2-trichloroethylchloroformate (0.31 mL, 2.2 mmol, 1.1 eq). The reaction mixture was stirred at room temperature for 18 h. Water was added, and the reaction mixture was extracted with ethyl acetate. The organic layers were combined, dried over magnesium sulfate, filtered and evaporated. The compound was obtained as orange powder (900 mg, quantitative yield) and was used without purification in the next step.

### Step2. Synthesis of Compound 1.4: 1-(3-(tert-butyl)-1-(4-chlorophenyl)-1H-pyrazol-5-yl)-3-(2-(methylthio)-4-((3-oxo-3,4-dihydropyrido[2,3-b]pyrazin-8-yl)oxy)phenyl)urea (Compound (ao))

According to Scheme 4 Step 2: In a sealed tube containing 8-(4-amino-3-(methylthio)phenoxy)pyrido[2,3-b]pyrazin-3(4H)-one, hydrochloride 111 (175 mg, 0.58 mmol, 1.0 eq) DIEA (6 eq) in DMSO (0.1M) was added 2,2,2-trichloroethyl N-[5-tert-butyl-2-(4-chlorophenyl)pyrazol-3-yl]carbamate (247 mg, 0.58 mmol, 1.0 eq). The reaction mixture was stirred at 70° C. for 18 h to afford after extraction, 2 flash chromatographies on silica gel (cyclohexane/ethyl acetate then ethyl acetate/methanol) and a semi-preparative HPLC to afford expected compound as white powder (Yield: 8%, 26 mg). MS m/z (ES) [M+H]⁺: 576.33

### Synthesis of Compound (ap): 5-(4-(3-(3-(tert-butyl)-1-phenyl-1H-pyrazol-5-yl)ureido)-3-(methylthio)phenoxy)-N-methylpicolinamide

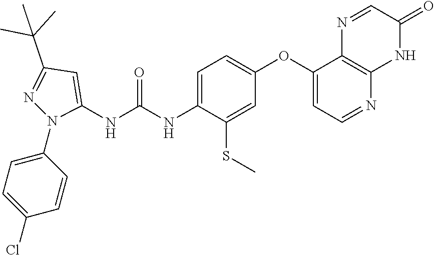 [SMILES: CSc1cc(Oc2ccnc3[nH]c(=O)cnc23)ccc1NC(=O)Nc1cc(C(C)(C)C)nn1-c1ccc(Cl)cc1]

### Synthesis of Compound 1.4: 5-(4-(3-(3-(tert-butyl)-1-phenyl-1H-pyrazol-5-yl)ureido)-3-(methylthio)phenoxy)-N-methylpicolinamide (Compound (ap))

According to Scheme 4 Step 2: In a sealed tube containing 5-(4-amino-3-methylsulfanyl-phenoxy)-N-methyl-pyridin-2-carboxamide 140 (110 mg, 0.38 mmol), DIEA (0.40 mL, 2.28 mmol) was added 2,2,2-trichloroethyl N-(5-tert-butyl-2-phenyl-pyrazol-3-yl)carbamate 158 (178 mg, 0.45 mmol). The reaction mixture was stirred at 70° C. for 24 h to afford after extraction, flash chromatography on silica gel (12 g, SiO₂, cyclohexane/AcOEt 100:0→0:100, 10 CV) and a semi-preparative HPLC to afford expected compound as white powder (Yield: 20%, 42 mg). MS m/z (ES) [M+H]⁺: 531.50

¹H NMR (300 MHz, DMSO-d₆) δ 8.95 (s, 1H), 8.63 (qd, J=4.7 Hz, 1H), 8.36 (d, J=2.7 Hz, 1H), 8.33 (s, 1H), 7.99 (d, J=8.7 Hz, 1H), 7.69 (d, J=8.8 Hz, 1H), 7.52-7.50 (m, 4H), 7.46-7.37 (m, 2H), 7.12 (d, J=2.7 Hz, 1H), 6.94 (dd, J=8.8, 2.7 Hz, 1H), 6.34 (s, 1H), 2.78 (d, J=4.8 Hz, 3H), 2.40 (s, 3H), 1.26 (s, 9H).

### Synthesis of Compound (aq): N-(4-(4-(3-(3-(tert-butyl)-1-phenyl-1H-pyrazol-5-yl)ureido)-3-(methylthio)phenoxy)pyridin-2-yl)acetamide

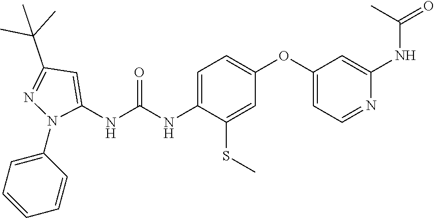 [SMILES: CSc1cc(Oc2ccnc(NC(C)=O)c2)ccc1NC(=O)Nc1cc(C(C)(C)C)nn1-c1ccccc1]

### Synthesis of Compound 1.4: N-(4-(4-(3-(3-(tert-butyl)-1-phenyl-1H-pyrazol-5-yl)ureido)-3-(methylthio)phenoxy)pyridin-2-yl)acetamide (Compound (aq))

According to Scheme 4 Step 2: In a sealed tube containing N-[4-(4-amino-3-methylsulfanyl-phenoxy)-2-pyridyl]acetamide 142 (64 mg, 0.22 mmol), DIEA (0.23 mL, 1.32 mmol) was added 2,2,2-trichloroethyl N-(5-tert-butyl-2-phenyl-pyrazol-3-yl) carbamate 158 (86 mg, 0.22 mmol). The reaction mixture was stirred at 70° C. for 6 h to afford after extraction, flash chromatography on silica gel (12 g, SiO₂, cyclohexane/AcOEt 100:0→0:100, 10 CV) and a semi-preparative HPLC to afford expected compound as white powder (Yield: 3%, 9 mg). MS m/z (ES) [M+H]⁺: 531.50

¹H NMR (300 MHz, DMSO-d₆) δ 10.52 (s, 1H), 9.01 (s, 1H), 8.37 (s, 1H), 8.17 (d, J=5.7 Hz, 1H), 7.75 (d, J=8.8 Hz, 1H), 6.67 (d, J=2.2 Hz, 1H), 7.55-7.53 (m, 4H), 7.44-7.39 (m, 1H), 7.13 (d, J=2.7 Hz, 1H), 6.96 (dd, J=8.8, 2.7 Hz, 1H), 6.65 (dd, J=5.7, 2.3 Hz, 1H), 6.37 (s, 1H), 2.43 (s, 3H), 2.04 (s, 3H), 1.28 (s, 9H).

### Synthesis of Compound (ar): 1-(3-(tert-butyl)-1-(pyridin-2-yl)-1H-pyrazol-5-yl)-3-(2-(methylthio)-4-((3-oxo-3,4-dihydropyrido[2,3-b]pyrazin-8-yl)oxy)phenyl)urea

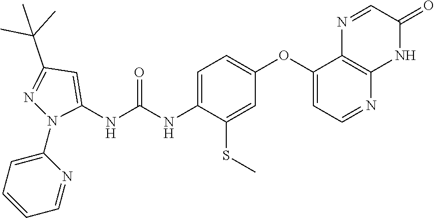 [SMILES: CSc1cc(Oc2ccnc3[nH]c(=O)cnc23)ccc1NC(=O)Nc1cc(C(C)(C)C)nn1-c1ccccn1]

### Step1. Synthesis of Compound 1.11

According to Scheme 4 Step 1: At 0° C., to a solution of N-[5-tert-butyl-2-(2-pyridyl)pyrazol-3-yl]amine (250 mg, 1.16 mmol, 1.0 eq) in dry THF (0.2M) was added dropwise dry pyridine (1.1 eq) followed by 2,2,2-trichloroethylchloroformate (0.18 mL, 1.3 mmol, 1.1 eq). The reaction mixture was stirred at room temperature for 18 h. Water was added, and the reaction mixture was extracted with ethyl acetate. The organic layers were combined, dried over magnesium sulfate, filtered and evaporated. The compound was obtained as brown gum (500 mg).

### Step2. Synthesis of Compound 1.4: 1-(3-(tert-butyl)-1-(pyridin-2-yl)-1H-pyrazol-5-yl)-3-(2-(methylthio)-4-((3-oxo-3,4-dihydropyrido[2,3-b]pyrazin-8-yl)oxy)phenyl)urea (Compound (ar))

According to Scheme 4 Step 2: In a sealed tube containing 8-(4-amino-3-(methylthio)phenoxy)pyrido[2,3-b]pyrazin-3(4H)-one, hydrochloride 111 (150 mg, 0.5 mmol, 1.0 eq) DIEA (6 eq) in DMSO (0.1M) was added 2,2,2-trichloroethyl N-[5-tert-butyl-2-(2-pyridyl)pyrazol-3-yl]carbamate (294 mg, 0.75 mmol, 1.5 eq). The reaction mixture was stirred at 70° C. for 18 h to afford after extraction, 2 flash chromatographies on silica gel (cyclohexane/ethyl acetate then ethyl acetate/methanol) and a semi-preparative HPLC to afford expected compound as brown powder (Yield: 3%, 7 mg). MS m/z (ES) [M+H]⁺: 543.33

### Synthesis of Compound (as): 1-(3-(tert-butyl)-1-(3-chlorophenyl)-1H-pyrazol-5-yl)-3-(2-(methylthio)-4-((3-oxo-3,4-dihydropyrido[2,3-b]pyrazin-8-yl)oxy)phenyl)urea

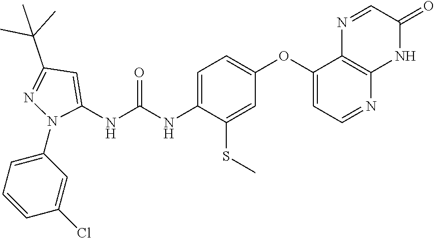 [SMILES: CSc1cc(Oc2ccnc3[nH]c(=O)cnc23)ccc1NC(=O)Nc1cc(C(C)(C)C)nn1-c1cccc(Cl)c1]

### Step1. Synthesis of Compound 1.11

According to Scheme 4 Step 1: At 0° C., to a solution of N-[5-tert-butyl-2-(3-chlorophenyl)pyrazol-3-yl]amine (500 mg, 2.00 mmol, 1.0 eq) in dry THF (0.2M) was added dropwise dry pyridine (1.1 eq) followed by 2,2,2-trichloroethylchloroformate (0.31 mL, 2.2 mmol, 1.1 eq). The reaction mixture was stirred at room temperature for 18 h. Water was added, and the reaction mixture was extracted with ethyl acetate. The organic layers were combined, dried over magnesium sulfate, filtered and evaporated. The compound was obtained as brown gum (927 mg, quantitative yield) and was used without purification in the next step.

### Step2. Synthesis of Compound 1.4: 1-(3-(tert-butyl)-1-(3-chlorophenyl)-1H-pyrazol-5-yl)-3-(2-(methylthio)-4-((3-oxo-3,4-dihydropyrido[2,3-b]pyrazin-8-yl)oxy)phenyl)urea (Compound (as))

According to Scheme 4 Step 2: In a sealed tube containing 8-(4-amino-3-(methylthio)phenoxy)pyrido[2,3-b]pyrazin-3(4H)-one, hydrochloride 111 (150 mg, 0.5 mmol, 1.0 eq) DIEA (6 eq) in DMSO (0.1M) was added 2,2,2-trichloroethyl N-[5-tert-butyl-2-(3-chlorophenyl)pyrazol-3-yl]carbamate (425 mg, 1.0 mmol, 2.0 eq). The reaction mixture was stirred at 70° C. for 2 days to afford after extraction, 2 flash chromatographies on silica gel (cyclohexane/ethyl acetate then ethyl acetate/methanol) and a semi-preparative HPLC to afford expected compound as brown powder (Yield: 4%, 11 mg). MS m/z (ES) [M+H]⁺: 576.33

### Synthesis of Compound (at): 1-(3-(tert-butyl)-1-(2-chlorophenyl)-1H-pyrazol-5-yl)-3-(2-(methylthio)-4-((3-oxo-3,4-dihydropyrido[2,3-b]pyrazin-8-yl)oxy)phenyl)urea

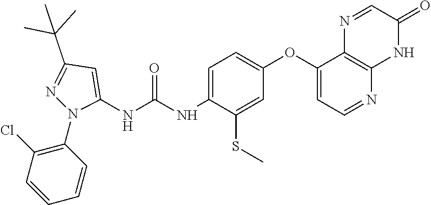 [SMILES: CSc1cc(Oc2ccnc3[nH]c(=O)cnc23)ccc1NC(=O)Nc1cc(C(C)(C)C)nn1-c1ccccc1Cl]

### Step1. Synthesis of Compound 1.11

According to Scheme 4 Step 1: At 0° C., to a solution of N-[5-tert-butyl-2-(2-chlorophenyl)pyrazol-3-yl]amine (500 mg, 2.00 mmol, 1.0 eq) in dry THF (0.2M) was added dropwise dry pyridine (1.1 eq) followed by 2,2,2-trichloroethylchloroformate (0.31 mL, 2.2 mmol, 1.1 eq). The reaction mixture was stirred at room temperature for 18 h. Water was added, and the reaction mixture was extracted with ethyl acetate. The organic layers were combined, dried over magnesium sulfate, filtered and evaporated. The compound was obtained as brown gum (933 mg, quantitative yield) and was used without purification in the next step.

### Step2. Synthesis of Compound 1.4: 1-(3-(tert-butyl)-1-(2-chlorophenyl)-1H-pyrazol-5-yl)-3-(2-(methylthio)-4-((3-oxo-3,4-dihydropyrido[2,3-b]pyrazin-8-yl)oxy)phenyl)urea (Compound (at))

According to Scheme 4 Step 2: In a sealed tube containing 8-(4-amino-3-(methylthio)phenoxy)pyrido[2,3-b]pyrazin-3(4H)-one, hydrochloride 111 (150 mg, 0.5 mmol, 1.0 eq) DIEA (6 eq) in DMSO (0.1M) was added 2,2,2-trichloroethyl N-[5-tert-butyl-2-(2-chlorophenyl)pyrazol-3-yl]carbamate (425 mg, 1.0 mmol, 2.0 eq). The reaction mixture was stirred at 70° C. for 2 days to afford after extraction, 2 flash chromatographies on silica gel (cyclohexane/ethyl acetate then ethyl acetate/methanol) and a semi-preparative HPLC to afford expected compound as brown powder (Yield: 10%, 28 mg). MS m/z (ES) [M+H]⁺: 576.33

### Synthesis of Compound (au): 1-(3-(tert-butyl)-1-phenyl-1H-pyrazol-5-yl)-3-(4-((4-methyl-3-oxo-3,4-dihydropyrido[2,3-b]pyrazin-8-yl)oxy)-2-(methylthio)phenyl)urea

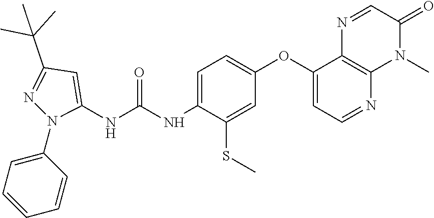 [SMILES: CSc1cc(Oc2ccnc3c2ncc(=O)n3C)ccc1NC(=O)Nc1cc(C(C)(C)C)nn1-c1ccccc1]

### Synthesis of Compound 1.4: 1-(3-(tert-butyl)-1-phenyl-1H-pyrazol-5-yl)-3-(4-((4-methyl-3-oxo-3,4-dihydropyrido[2,3-b]pyrazin-8-yl)oxy)-2-(methylthio)phenyl)urea (Compound (au))

According to Scheme 4 Step 2: In a sealed tube containing 8-(4-amino-3-methylsulfanyl-phenoxy)-4-methyl-pyrido[2,3-b] pyrazin-3-one hydrochloride 144 (222 mg, 0.63 mmol), DIEA (1.10 mL, 6.3 mmol) in DMSO (0.1M) was added 2,2,2-trichloroethyl N-(5-tert-butyl-2-phenyl-pyrazol-3-yl)carbamate 158 (370 mg, 0.95 mmol). The reaction mixture was stirred at 70° C. for 6 h to afford after extraction, flash chromatography on silica gel (12 g, SiO₂, cyclohexane/AcOEt 100:0→0:100, 10 CV) and a semi-preparative HPLC to afford expected compound as yellow powder (Yield: 25%, 88 mg). MS m/z (ES) [M+H]⁺: 556.42

¹H NMR (300 MHz, DMSO-d₆) δ 8.98 (s, 1H), 8.45 (d, J=5.6 Hz, 1H), 8.37 (s, 1H), 8.26 (s, 1H), 7.75 (d, J=8.8 Hz, 1H), 7.53-7.50 (m, 4H), 7.42-7.36 (m, 1H), 7.18 (d, J=2.7 Hz, 1H), 7.01 (dd, J=8.8, 2.7 Hz, 1H), 6.67 (d, J=5.6 Hz, 1H), 6.35 (s, 1H), 3.64 (s, 3H), 2.42 (s, 3H), 1.26 (s, 9H).

### Synthesis of Compound (av): 1-(3-(tert-butyl)-1-(2-methoxyphenyl)-1H-pyrazol-5-yl)-3-(2-(methylthio)-4-((3-oxo-3,4-dihydropyrido[2,3-b]pyrazin-8-yl)oxy)phenyl)urea

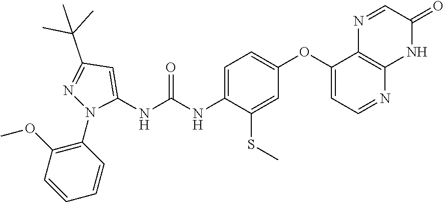 [SMILES: COc1ccccc1-n1nc(C(C)(C)C)cc1NC(=O)Nc1ccc(Oc2ccnc3[nH]c(=O)cnc23)cc1SC]

### Step1. Synthesis of Compound 1.11

According to Scheme 4 Step 1: At 0° C., to a solution of N-[5-tert-butyl-2-(2-methoxyphenyl)pyrazol-3-yl]amine (500 mg, 2.04 mmol, 1.0 eq) in dry THF (0.2M) was added dropwise dry pyridine (1.1 eq) followed by 2,2,2-trichloroethylchloroformate (0.31 mL, 2.24 mmol, 1.1 eq). The reaction mixture was stirred at room temperature for 18 h. Water was added, and the reaction mixture was extracted with ethyl acetate. The organic layers were combined, dried over magnesium sulfate, filtered and evaporated. The compound was obtained as white solid (1.2 g, quantitative yield) and was used without purification in the next step.

### Step2. Synthesis of Compound 1.4: 1-(3-(tert-butyl)-1-(2-methoxyphenyl)-1H-pyrazol-5-yl)-3-(2-(methylthio)-4-((3-oxo-3,4-dihydropyrido[2,3-b]pyrazin-8-yl)oxy)phenyl)urea (Compound (av))

According to Scheme 4 Step 2: In a sealed tube containing 8-(4-amino-3-(methylthio)phenoxy)pyrido[2,3-b]pyrazin-3(4H)-one, hydrochloride 111 (150 mg, 0.5 mmol, 1.0 eq) DIEA (6 eq) in DMSO (0.1M) was added 2,2,2-trichloroethyl N-[5-tert-butyl-2-(2-methoxyphenyl)pyrazol-3-yl]carbamate (563 mg, 1.34 mmol, 2.0 eq). The reaction mixture was stirred at 70° C. for 2 days to afford after extraction, 2 flash chromatographies on silica gel (cyclohexane/ethyl acetate then ethyl acetate/methanol) and a semi-preparative HPLC to afford expected compound as white powder (Yield: 13%, 34 mg). MS m/z (ES) [M+H]⁺: 572.33

### Synthesis of Compound (aw): 1-(3-(tert-butyl)-1-(3-methoxyphenyl)-1H-pyrazol-5-yl)-3-(2-(methylthio)-4-((3-oxo-3,4-dihydropyrido[2,3-b]pyrazin-8-yl)oxy)phenyl)urea

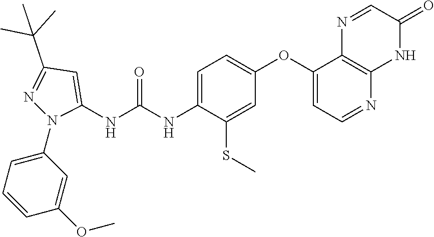 [SMILES: COc1cccc(-n2nc(C(C)(C)C)cc2NC(=O)Nc2ccc(Oc3ccnc4[nH]c(=O)cnc34)cc2SC)c1]

### Step1. Synthesis of Compound 1.11

According to Scheme 4 Step 1: At 0° C., to a solution of N-[5-tert-butyl-2-(3-methoxyphenyl)pyrazol-3-yl]amine (500 mg, 2.04 mmol, 1.0 eq) in dry THF (0.2M) was added dropwise dry pyridine (1.1 eq) followed by 2,2,2-trichloroethylchloroformate (0.31 mL, 2.24 mmol, 1.1 eq). The reaction mixture was stirred at room temperature for 18 h. Water was added, and the reaction mixture was extracted with ethyl acetate. The organic layers were combined, dried over magnesium sulfate, filtered and evaporated. The compound was obtained as white solid (1.2 g, quantitative yield) and was used without purification in the next step.

### Step2. Synthesis of Compound 1.4: 1-(3-(tert-butyl)-1-(3-methoxyphenyl)-1H-pyrazol-5-yl)-3-(2-(methylthio)-4-((3-oxo-3,4-dihydropyrido[2,3-b]pyrazin-8-yl)oxy)phenyl)urea (Compound (aw))

According to Scheme 4 Step 2: In a sealed tube containing 8-(4-amino-3-(methylthio)phenoxy)pyrido[2,3-b]pyrazin-3(4H)-one, hydrochloride 111 (150 mg, 0.5 mmol, 1.0 eq) DIEA (6 eq) in DMSO (0.1M) was added 2,2,2-trichloroethyl N-[5-tert-butyl-2-(3-methoxyphenyl)pyrazol-3-yl]carbamate (281 mg, 0.67 mmol, 1.5 eq). The reaction mixture was stirred at 70° C. for 2 days to afford after extraction, 2 flash chromatographies on silica gel (cyclohexane/ethyl acetate then ethyl acetate/methanol) and a semi-preparative HPLC to afford expected compound as white powder (Yield: 25%, 63 mg). MS m/z (ES) [M+H]⁺: 572.33

### Synthesis of Compound (ax): 1-(4-(3,4-difluorophenyl)thiazol-2-yl)-3-(2-(methylthio)-4-((3-oxo-3,4-dihydropyrido[2,3-b]pyrazin-8-yl)oxy)phenyl)urea

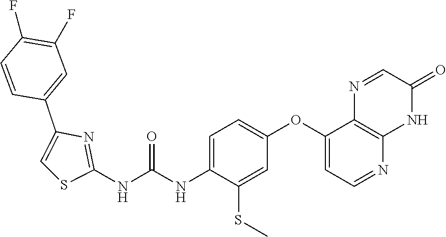 [SMILES: CSc1cc(Oc2ccnc3[nH]c(=O)cnc23)ccc1NC(=O)Nc1nc(-c2ccc(F)c(F)c2)cs1]

### Step1. Synthesis of Compound 1.2

According to Scheme 1 Step 1: Triphosgene (1.1 eq) was added dropwise at room temperature to a solution of 4-(3,4-difluorophenyl)thiazol-2-amine (118 mg, 0.55 mmol, 1.1 eq) in dry THF. Triethylamine (2.3 eq) was then added and the mixture was heated at 80° C. in a sealed tube for 2 h. After cooling down, the solvent was evaporated, and the residue was dissolved in a 50/50 mixture of toluene and acetonitrile.

### Step2. Synthesis of Compound 1.4: 1-(4-(3,4-difluorophenyl)thiazol-2-yl)-3-(2-(methylthio)-4-((3-oxo-3,4-dihydropyrido[2,3-b]pyrazin-8-yl)oxy)phenyl)urea (Compound (ax))

According to Scheme 1 Step 2: 8-(4-amino-3-(methylthio)phenoxy)pyrido[2,3-b]pyrazin-3(4H)-one, hydrochloride 111 (168 mg, 0.50 mmol, 1.0 eq) was added and the reaction mixture was stirred at 120° C. for 3 h. After cooling down, the solvents were evaporated and the residue was triturated in water, filtrated and purified by semi-preparative HPLC to afford expected compound as beige powder (Yield: 7%, 20 mg). MS m/z (ES) [M+H]⁺: 539.17

### Synthesis of Compound (ay): 1-(2-(methylthio)-4-((3-oxo-3,4-dihydropyrido[2,3-b]pyrazin-8-yl)oxy)phenyl)-3-(5-phenyl-1,3,4-thiadiazol-2-yl)urea

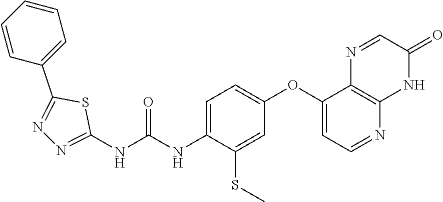 [SMILES: CSc1cc(Oc2ccnc3[nH]c(=O)cnc23)ccc1NC(=O)Nc1nnc(-c2ccccc2)s1]

### Step1. Synthesis of Compound 1.2

According to Scheme 1 Step 1: Triphosgene (1.1 eq) was added dropwise at room temperature to a solution of 5-phenyl-1,3,4-thiadiazol-2-amine (97 mg, 0.55 mmol, 1.1 eq) in dry THF. Triethylamine (2.3 eq) was then added and the mixture was heated at 80° C. in a sealed tube for 2 h. After cooling down, the solvent was evaporated, and the residue was dissolved in a 50/50 mixture of toluene and acetonitrile.

### Step2. Synthesis of Compound 1.4: 1-(2-(methylthio)-4-((3-oxo-3,4-dihydropyrido[2,3-b]pyrazin-8-yl)oxy)phenyl)-3-(5-phenyl-1,3,4-thiadiazol-2-yl)urea (Compound (ay))

According to Scheme 1 Step 2: 8-(4-amino-3-(methylthio)phenoxy)pyrido[2,3-b]pyrazin-3(4H)-one, hydrochloride 111 (168 mg, 0.50 mmol, 1.0 eq) was added and the reaction mixture was stirred at 120° C. for 3 h. After cooling down, the solvents were evaporated and the residue was triturated in water, filtrated and purified by semi-preparative HPLC to afford expected compound as beige powder (Yield: 9%, 29 mg). MS m/z (ES) [M+H]⁺: 504.17

### Synthesis of Compound (az): 1-(3-(tert-butyl)-1-(p-tolyl)-1H-pyrazol-5-yl)-3-(4-((3-oxo-3,4-dihydropyrido[2,3-b]pyrazin-8-yl)oxy)-2-(trifluoromethyl)phenyl)urea

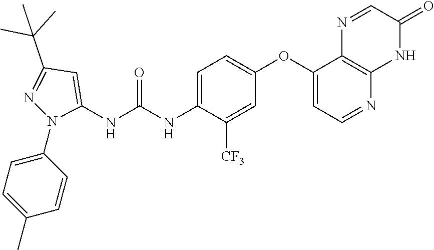 [SMILES: Cc1ccc(-n2nc(C(C)(C)C)cc2NC(=O)Nc2ccc(Oc3ccnc4[nH]c(=O)cnc34)cc2C(F)(F)F)cc1]

### Step1. Synthesis of Compound 1.2

According to Scheme 1 Step 1: A solution of tert-butyl-2-p-tolyl-2H-pyrazol-3-ylamine 306 (126 mg, 0.55 mmol) dissolved in THF (5 mL) was slowly added into a stirred solution of triphosgene (163 mg, 0.55 mmol) in THF (2 mL) at room temperature. Triethylamine (0.16 mL, 1.15 mmol) was then added slowly to the reaction mixture. After 2 h at reflux, the solvent was removed under vacuum. The residue was dissolved in acetonitrile (5 mL) and toluene (5 mL).

### Step2. Synthesis of Compound 1.4: 1-(3-(tert-butyl)-1-(p-tolyl)-1H-pyrazol-5-yl)-3-(4-((3-oxo-3,4-dihydropyrido[2,3-b]pyrazin-8-yl)oxy)-2-(trifluoromethyl)phenyl)urea (Compound (az))

According to Scheme 1 Step 2: Then 8-[4-amino-3-(trifluoromethyl)phenoxy]-4H-pyrido[2,3-b]pyrazin-3-one hydrochloride 149 (161 mg, 0.50 mmol) was added and the reaction mixture was stirred at 120° C. for 3 h. After cooling down, the solvents were evaporated and the residue was triturated in water, filtrated and purified by semi-preparative HPLC to afford expected compound as ink solid (Yield: 8%, 23 mg). MS m/z (ES) [M+H]⁺: 578.42

¹H NMR (300 MHz, DMSO-d₆) δ 12.95 (s, 1H), 8.98 (s, 1H), 8.44 (s, 1H), 8.37 (d, J=5.6 Hz, 1H), 8.16 (d, J=2.0 Hz, 1H), 7.84 (d, J=8.9 Hz, 1H), 7.55 (d, J=2.8 Hz, 1H), 7.51-7.30 (m, 5H), 6.67 (d, J=5.6 Hz, 1H), 6.32 (s, 1H), 2.36 (s, 3H), 1.25 (s, 9H).

### Synthesis of Compound (ba): 1-(5-(tert-butyl)thiazol-2-yl)-3-(4-((3-oxo-3,4-dihydropyrido[2,3-b]pyrazin-8-yl)oxy)naphthalen-1-yl)urea

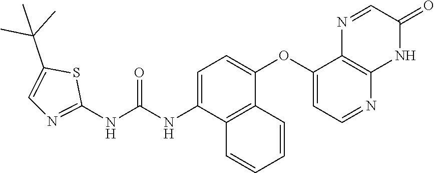 [SMILES: CC(C)(C)c1cnc(NC(=O)Nc2ccc(Oc3ccnc4[nH]c(=O)cnc34)c3ccccc23)s1]

### Synthesis of Compound 1.4: 1-(5-(tert-butyl)thiazol-2-yl)-3-(4-((3-oxo-3,4-dihydropyrido[2,3-b]pyrazin-8-yl)oxy)naphthalen-1-yl)urea (Compound (ba))

According to Scheme 4 Step 2: In a sealed tube containing 8-[(4-amino-1-naphthyl)oxy]-4H-pyrido[2,3-b]pyrazin-3-one hydrochloride 155 (140 mg, 0.41 mmol), DIEA (0.7 mL, 4.1 mmol) in DMSO (0.1M) was added 2,2,2-trichloroethyl N-(5-tert-butylthiazol-2-yl)carbamate 157 (273 mg, 0.82 mmol). The reaction mixture was stirred at 70° C. for 24 h to afford after extraction, flash chromatography on silica gel (12 g, SiO₂, cyclohexane/AcOEt 100:0→0:100, 10 CV) and a semi-preparative HPLC to afford expected compound as white powder (Yield: 5%, 18 mg). MS m/z (ES) [M+H]⁺: 487.42

¹H NMR (300 MHz, DMSO-d₆) δ 12.93 (s, 1H), 10.72 (s, 1H), 9.30 (s, 1H), 8.25 (d, J=5.7 Hz, 1H), 8.24 (s, 1H), 8.17 (d, J=8.6 Hz, 1H), 8.06 (d, J=8.3 Hz, 1H), 7.86 (d, J=7.9 Hz, 1H), 7.72-7.55 (m, 2H), 7.40 (d, J=8.4 Hz, 1H), 7.09 (s, 1H), 6.40 (d, J=5.6 Hz, 1H), 1.31 (s, 9H).

### Synthesis of Compound (bb): 1-(3-(tert-butyl)-1-(quinolin-6-yl)-1H-pyrazol-5-yl)-3-(4-((3-oxo-3,4-dihydropyrido[2,3-b]pyrazin-8-yl)oxy)naphthalen-1-yl)urea

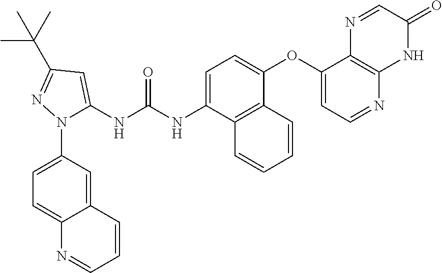 [SMILES: CC(C)(C)c1cc(NC(=O)Nc2ccc(Oc3ccnc4[nH]c(=O)cnc34)c3ccccc23)n(-c2ccc3ncccc3c2)n1]

### Synthesis of Compound 1.4: 1-(3-(tert-butyl)-1-(quinolin-6-yl)-1H-pyrazol-5-yl)-3-(4-((3-oxo-3,4-dihydropyrido[2,3-b]pyrazin-8-yl)oxy)naphthalen-1-yl)urea (Compound (bb))

According to Scheme 4 Step 2: In a sealed tube containing 8-[(4-amino-1-naphthyl)oxy]-4H-pyrido[2,3-b]pyrazin-3-one hydrochloride 155 (140 mg, 0.41 mmol), DIEA (0.7 ml, 4.1 mmol) in DMSO (0.1M) was added 2,2,2-trichloroethyl N-[5-tert-butyl-2-(quinolyl)pyrazol-3-yl]carbamate 98 (373 mg, 0.84 mmol). The reaction mixture was stirred at 70° C. for 24 h to afford after extraction, flash chromatography on silica gel (12 g, SiO₂, cyclohexane/AcOEt 100:0→80:20, 10 CV) and a semi-preparative HPLC to afford expected compound as white powder (Yield: 21%, 54 mg). MS m/z (ES) [M+H]⁺: 597.33

¹H NMR (300 MHz, DMSO-d₆) δ 12.92 (s, 1H), 9.12 (s, 1H), 8.97 (s, 1H), 8.95 (d, J=5.9 Hz, 1H), 8.46 (d, J=5.7 Hz, 1H), 8.24-8.19 (m, 3H), 8.15 (d, J=8.6 Hz, 1H), 8.03-8.00 (m, 2H), 7.90 (d, J=8.3 Hz, 1H), 7.80 (d, J=8.3 Hz, 1H), 7.63-7.54 (m, 3H), 7.35 (d, J=8.3 Hz, 1H), 6.48 (s, 1H), 6.35 (d, J=5.7 Hz, 1H), 1.31 (s, 9H).

### Synthesis of Compound (bc): 1-(3-(tert-butyl)-1-phenyl-1H-pyrazol-5-yl)-3-(2-methoxy-4-((3-oxo-3,4-dihydropyrido[2,3-b]pyrazin-8-yl)oxy)phenyl)urea

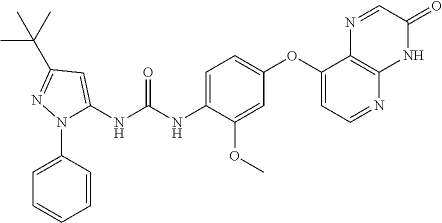 [SMILES: COc1cc(Oc2ccnc3[nH]c(=O)cnc23)ccc1NC(=O)Nc1cc(C(C)(C)C)nn1-c1ccccc1]

### Synthesis of Compound 1.4: 1-(3-(tert-butyl)-1-phenyl-1H-pyrazol-5-yl)-3-(2-methoxy-4-((3-oxo-3,4-dihydropyrido[2,3-b]pyrazin-8-yl)oxy)phenyl)urea (Compound (bc))

According to Scheme 4 Step 2: In a sealed tube containing 8-(4-amino-3-methoxy-phenoxy)-4H-pyrido[2,3-b]pyrazin-3-one hydrochloride 164 (200 mg, 0.7 mmol, 1.0 eq), DIEA (1.2 mL, 7.0 mmol, 10.0 eq) in DMSO (0.1M) was added 2,2,2,-trichloroethyl N-(5-tert-butyl-2-phenyl-pyrazol-3-yl)carbamate 158 (547 mg, 1.4 mmol, 2.0 eq). The reaction mixture was stirred at 70° C. for 2 days. The expected compound was obtained after 2 flash chromatographies (cyclohexane/ethyl acetate then ethyl acetate/methanol) and semi-preparative HPLC as pale brown solid (Yield: 6%, 23 mg). MS m/z (ES) [M+H]⁺: 526.42

### Synthesis of Compound (bd): 1-(3-(tert-butyl)-1-(6-methylpyridin-3-yl)-1H-pyrazol-5-yl)-3-(2-(methylthio)-4-((3-oxo-3,4-dihydropyrido[2,3-b]pyrazin-8-yl)oxy)phenyl)urea

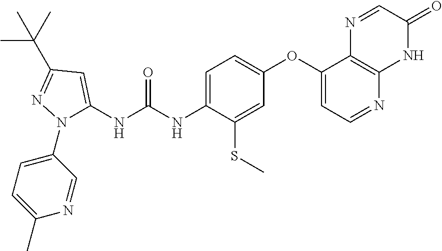 [SMILES: CSc1cc(Oc2ccnc3[nH]c(=O)cnc23)ccc1NC(=O)Nc1cc(C(C)(C)C)nn1-c1ccc(C)nc1]

### Step1. Synthesis of Compound 1.10

According to Scheme 3 Step 1: Under Argon, triphosgene (0.130 g, 0.44 mmol) was added portionwise to a solution of 111 (150 mg, 0.44 mmol, 1.0 eq) and Et₃N (0.075 mL, 0.542 mmol) in CH₂Cl₂ (5 mL) at −20° C., the reaction mass was stirred at room temperature for 1 h. Then the solvent was removed under reduced pressure, THF (5 mL) was added to the residue, the formed precipitate was filtered.

### Step 2. Synthesis of: 1-(3-(tert-butyl)-1-(6-methylpyridin-3-yl)-1H-pyrazol-5-yl)-3-(2-(methylthio)-4-((3-oxo-3,4-dihydropyrido[2,3-b]pyrazin-8-yl)oxy)phenyl)urea (Compound (bd))

According to Scheme 3 Step 2: Then a solution of 5-tert-butyl-2-[6-methyl-3-pyridyl]pyrazol-3-amine 166 (91 mg, 0.4 mmol, 0.9 eq) in CH₂Cl₂ (5 mL) was added dropwise at 0° C. The reaction mixture was stirred at room temperature for 20 h. The residue was purified by semi-preparative HPLC to afford, after basic extraction, expected compound as white powder (Yield: 16%, 40 mg). MS m/z (ES) [M+H]⁺: 557.33

### Synthesis of Compound (be): 1-(3-(tert-butyl)-1-phenyl-1H-pyrazol-5-yl)-3-(2-(methylthio)-4-((5,6,7,8-tetrahydro-1,8-naphthyridin-4-yl)oxy)phenyl)urea

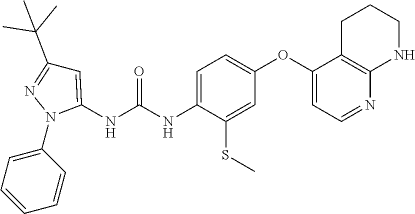 [SMILES: CSc1cc(Oc2ccnc3c2CCCN3)ccc1NC(=O)Nc1cc(C(C)(C)C)nn1-c1ccccc1]

### Synthesis of Compound 1.4: 1-(3-(tert-butyl)-1-phenyl-1H-pyrazol-5-yl)-3-(2-(methylthio)-4-((5,6,7,8-tetrahydro-1,8-naphthyridin-4-yl)oxy)phenyl)urea (Compound (be))

NaBH₄ (15 mg, 0.41 mmol) was added to a solution of 1-(3-(tert-butyl)-1-phenyl-1H-pyrazol-5-yl)-3-(2-(methylthio)-4-((7-oxo-5,6,7,8-tetrahydro-1,8-naphthyridin-4-yl)oxy)phenyl)urea (an) (45 mg, 0.08 mmol) in dry THF (2 mL). The mixture was cooled at 0° C. under argon before addition of BF₃ etherate 47% (0.15 mL, 0.58 mmol). The reaction mixture was stirred at room temperature for 3 h. Then, aq. sat. NH₄Cl (20 mL) and AcOEt (30 mL) were added. The aqueous layer was extracted with AcOEt (2×20 mL). The combined organic fractions were dried over MgSO₄, filtered and concentrated in vacuo. The crude obtained was purified by semi-preparative HPLC leading to the title compound (Yield: 52%, 22 mg) as a white solid. MS m/z (ES) [M+H]⁺: 529.42

¹H NMR (300 MHz, DMSO-d₆) δ 8.92 (s, 1H), 8.30 (s, 1H), 7.63 (d, J=5.6 Hz, 1H), 7.62 (d, J=8.8 Hz, 1H), 7.52-7.50 (m, 4H), 7.53-7.49 (m, 1H), 6.99 (d, J=2.7 Hz, 1H), 6.80 (dd, J=8.8, 2.7 Hz, 1H), 6.49 (s, 1H), 6.33 (s, 1H), 5.81 (d, J=5.7 Hz, 1H), 3.22 (br s, 2H), 2.62 (t, J=6.1 Hz, 2H), 2.39 (s, 3H), 1.79-1.71 (m, 2H), 1.25 (s, 9H).

### Synthesis of Compound (bf): 4-((4-(3-(3-(tert-butyl)-1-(quinolin-6-yl)-1H-pyrazol-5-yl)ureido)naphthalen-1-yl)oxy)-N-methylpicolinamide

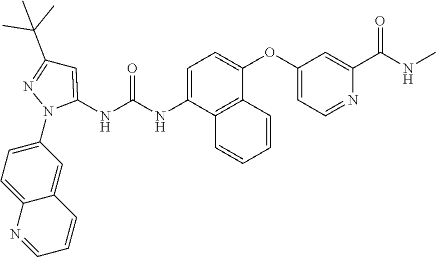 [SMILES: CNC(=O)c1cc(Oc2ccc(NC(=O)Nc3cc(C(C)(C)C)nn3-c3ccc4ncccc4c3)c3ccccc23)ccn1]

### Synthesis of Compound 1.4: 4-((4-(3-(3-(tert-butyl)-1-(quinolin-6-yl)-1H-pyrazol-5-yl)ureido)naphthalen-1-yl)oxy)-N-methylpicolinamide (Compound (bf))

According to Scheme 4 Step 2: In a sealed tube containing 4-[(4-amino-1-naphthyl)oxy]-N-methyl-pyridine-2-carboxamide 167 (115 mg, 0.40 mmol), DIEA (0.40 mL, 2.35 mmol)) in DMSO (0.1M) was added 2,2,2-trichloroethyl N-[5-tert-butyl-2-(6-quinolyl)pyrazol-3-yl]carbamate 98 (340 mg, 0.78 mmol). The reaction mixture was stirred at 70° C. for 18 h to afford after extraction, flash chromatography on silica gel (12 g, SiO₂, cyclohexane/AcOEt 100:0→80:20, 5 CV) and a semi-preparative HPLC to afford expected compound as white powder (Yield: 20%, 48 mg). MS m/z (ES) [M+H]⁺: 586.42

¹H NMR (300 MHz, DMSO-d₆) δ 9.14 (s, 1H), 9.04 (s, 1H), 8.94 (dd, J=4.2, 1.6 Hz, 1H), 8.75 (qd, J=4.9 Hz, 1H), 8.51-8.48 (m, 2H), 8.22-8.17 (m, 2H), 8.04-7.98 (m, 2H), 7.93 (d, J=8.3 Hz, 1H), 7.79-7.76 (m, 1H), 7.63-7.53 (m, 3H), 7.37 (d, J=8.3 Hz, 1H), 7.29 (d, J=2.6 Hz, 1H), 7.17 (dd, J=5.6, 2.6 Hz, 1H), 6.49 (s, 1H), 2.72 (d, J=4.8 Hz, 3H), 1.31 (s, 9H).

### Synthesis of Compound (bg): 1-(3-(tert-butyl)-1-(2-(dimethylamino)ethyl)-1H-pyrazol-5-yl)-3-(2-(methylthio)-4-((3-oxo-3,4-dihydropyrido[2,3-b]pyrazin-8-yl)oxy)phenyl)urea

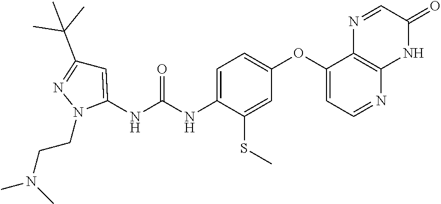 [SMILES: CSc1cc(Oc2ccnc3[nH]c(=O)cnc23)ccc1NC(=O)Nc1cc(C(C)(C)C)nn1CCN(C)C]

### Step1. Synthesis of Compound 1.10

According to Scheme 3 Step 1: Under Argon, triphosgene (0.130 g, 0.44 mmol) was added portionwise to a solution of 111 (150 mg, 0.44 mmol, 1.0 eq) and Et₃N (0.075 mL, 0.542 mmol) in CH₂Cl₂ (5 mL) at −20° C., the reaction mass was stirred at room temperature for 1 h. Then the solvent was removed under reduced pressure, THF (5 mL) was added to the residue, the formed precipitate was filtered.

### Step 2. Synthesis of: 1-(3-(tert-butyl)-1-(2-(dimethylamino)ethyl)-1H-pyrazol-5-yl)-3-(2-(methylthio)-4-((3-oxo-3,4-dihydropyrido[2,3-b]pyrazin-8-yl)oxy)phenyl)urea (Compound (bg))

According to Scheme 3 Step 2: Then a solution of 5-tert-butyl-2-[2-(dimethylamino)ethyl]pyrazol-3-amine 169 (101 mg, 0.48 mmol, 1.1 eq) in CH₂Cl₂ (5 mL) was added dropwise at 0° C. The reaction mixture was stirred at room temperature for 20 h. The residue was purified by semi-preparative HPLC to afford, after basic extraction, expected compound as off-white powder (Yield: 19%, 45 mg). MS m/z (ES) [M+H]⁺: 537.42

### Synthesis of Compound (bh): 1-(3-(tert-butyl)-1-(3-morpholinopropyl)-1H-pyrazol-5-yl)-3-(2-(methylthio)-4-((3-oxo-3,4-dihydropyrido[2,3-b]pyrazin-8-yl)oxy)phenyl)urea

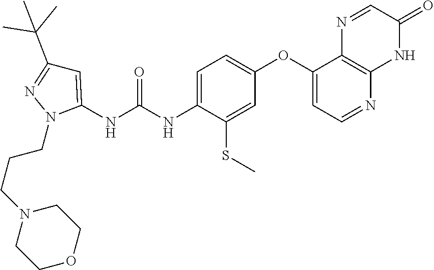 [SMILES: CSc1cc(Oc2ccnc3[nH]c(=O)cnc23)ccc1NC(=O)Nc1cc(C(C)(C)C)nn1CCCN1CCOCC1]

### Step1. Synthesis of Compound 1.10

According to Scheme 3 Step 1: Under Argon, triphosgene (0.130 g, 0.44 mmol) was added portionwise to a solution of 111 (150 mg, 0.44 mmol, 1.0 eq) and Et₃N (0.075 mL, 0.542 mmol) in CH₂Cl₂ (5 mL) at −20° C., the reaction mass was stirred at room temperature for 1 h. Then the solvent was removed under reduced pressure, THF (5 mL) was added to the residue, the formed precipitate was filtered.

### Step 2. Synthesis of: 1-(3-(tert-butyl)-1-(3-morpholinopropyl)-1H-pyrazol-5-yl)-3-(2-(methylthio)-4-((3-oxo-3,4-dihydropyrido[2,3-b]pyrazin-8-yl)oxy)phenyl)urea (Compound (bh))

According to Scheme 3 Step 2: Then a solution of 5-tert-butyl-2-[3-morpholinopropyl]pyrazol-3-amine 171 (128 mg, 0.48 mmol, 1.1 eq) in CH₂Cl₂ (5 mL) was added dropwise at 0° C. The reaction mixture was stirred at room temperature for 20 h. The residue was purified by semi-preparative HPLC to afford, after basic extraction, expected compound as yellow powder (Yield: 30%, 77 mg). MS m/z (ES) [M+H]⁺: 593.42

### Synthesis of Compound (bi): Tert-Butyl (4-(4-(3-(3-(tert-butyl)-1-phenyl-1H-pyrazol-5-yl)ureido)-3-(methylthio)phenoxy)pyridin-211)carbamate

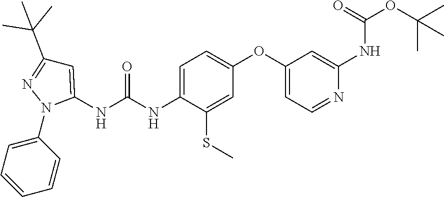 [SMILES: CSc1cc(Oc2ccnc(NC(=O)OC(C)(C)C)c2)ccc1NC(=O)Nc1cc(C(C)(C)C)nn1-c1ccccc1]

### Synthesis of Compound 1.4: Tert-Butyl (4-(4-(3-(3-(tert-butyl)-1-phenyl-1H-pyrazol-5-yl)ureido)-3-(methylthio)phenoxy)pyridin-2-yl)carbamate (Compound (bi))

According to Scheme 1 Step 2: On the tert-Butyl N-[4-(4-amino-3-methylsulfanyl-phenoxy)-2-pyridyl] carbamate 173 (192 mg, 0.50 mmol) dissolved in acetonitrile (5 mL) and toluene (5 mL), was added 54 (184 mg, 0.55 mmol). The reaction mixture was stirred at 80° C. for 18 h. After cooling down, the solvents were evaporated. The residue was purified by semi-preparative HPLC leading to title compound (Yield: 6%, 19 mg) as a brown solid. MS m/z (ES) [M+H]⁺: 561.42

¹H NMR (300 MHz, DMSO-d₆) δ 9.79 (s, 1H), 8.99 (s, 1H), 8.37 (s, 1H), 8.09 (d, J=5.7 Hz, 1H), 7.73 (d, J=8.8 Hz, 1H), 7.53-7.51 (m, 4H), 7.42-7.37 (m, 1H), 7.35 (d, J=2.2 Hz, 1H), 7.12 (d, J=2.7 Hz, 1H), 6.96 (dd, J=8.8, 2.7 Hz, 1H), 6.57 (dd, J=5.7, 2.3 Hz, 1H), 6.35 (s, 1H), 2.41 (s, 3H), 1.40 (s, 9H), 1.26 (s, 9H).

### Synthesis of Compound (bj): 1-(4-((2-aminopyridin-4-yl)oxy)-2-(methylthio)phenyl)-3-(3-(tert-butyl)-1-phenyl-1H-pyrazol-5-yl)urea Hydrochloride

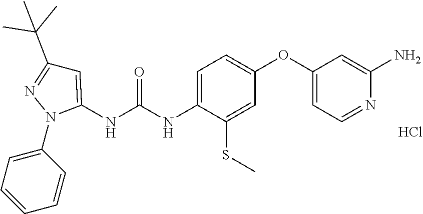 [SMILES: CSc1cc(Oc2ccnc(N)c2)ccc1NC(=O)Nc1cc(C(C)(C)C)nn1-c1ccccc1 ; Cl]

### Synthesis of: 1-(4-((2-aminopyridin-4-yl)oxy)-2-(methylthio)phenyl)-3-(3-(tert-butyl)-1-phenyl-1H-pyrazol-5-yl)urea hydrochloride (Compound (bj))

tert-Butyl N-[4-{4-[(5-tert-butyl-2-phenyl-pyrazol-3-yl)carbamoylamino]-3-methylsulfanyl-phenoxy}-2-pyridyl]carbamate (bi) (94 mg, 0.16 mmol) was treated with 4N HCl in dioxane (5 mL) at room temperature for 4 h. The solvent was removed under vacuum leading after lyophilization to the expected product (84 mg, quantitative yield) as a yellow foam. MS m/z (ES) [M+H]⁺: 489.42

¹H NMR (300 MHz, DMSO-d₆) δ 13.09 (br s, 1H), 9.08 (s, 1H), 8.44 (s, 1H), 7.92 (d, J=7.2 Hz, 1H), 7.79-7.75 (m, 2H), 7.53-7.48 (m, 4H), 7.43-7.36 (m, 1H), 7.20 (d, J=2.7 Hz, 1H), 7.06 (dd, J=8.8, 2.7 Hz, 1H), 6.64 (dd, J=7.2, 2.5 Hz, 1H), 6.34 (s, 1H), 6.09 (d, J=2.5 Hz, 1H), 2.43 (s, 3H), 1.26 (s, 9H).

### Synthesis of Compound (bk): 1-(3-(tert-butyl)-1-phenyl-1H-pyrazol-5-yl)-3-(2-isopropyl-4-((3-oxo-3,4-dihydropyrido[2,3-b]pyrazin-8-yl)oxy)phenyl)urea

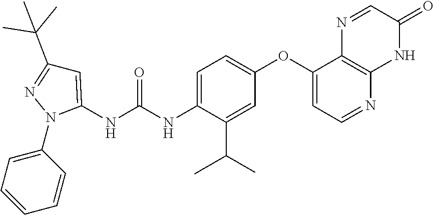 [SMILES: CC(C)c1cc(Oc2ccnc3[nH]c(=O)cnc23)ccc1NC(=O)Nc1cc(C(C)(C)C)nn1-c1ccccc1]

### Synthesis of Compound 1.4: 1-(3-(tert-butyl)-1-phenyl-1H-pyrazol-5-yl)-3-(2-isopropyl-4-((3-oxo-3,4-dihydropyrido[2,3-b]pyrazin-8-yl)oxy)phenyl)urea (Compound (bk))

According to Scheme 4 Step 2: In a sealed tube containing 8-(4-amino-3-isopropyl-phenoxy)-4H-pyrido[2,3-b]pyrazin-3-one hydrochloride 178 (333 mg, 1.0 mmol), DIEA (1.74 mL, 10.0 mmol) in DMSO (0.1M) was added 2,2,2-trichloroethyl N-(5-tert-butyl-2-phenyl-pyrazol-3-yl)carbamate 158 (781 mg, 2.0 mmol). The reaction mixture was stirred at 70° C. for 18 h to afford after extraction, flash chromatography on silica gel (40 g, SiO₂, cyclohexane/AcOEt 100:0→80:20, 10 CV) and a semi-preparative HPLC to afford expected compound as white powder (Yield: 3%, 125 mg). MS m/z (ES) [M+H]⁺: 538.42

¹H NMR (300 MHz, DMSO-d₆) δ 12.90 (s, 1H), 8.64 (s, 1H), 8.35-8.30 (m, 2H), 8.16 (s, 1H), 7.58 (d, J=8.8 Hz, 1H), 7.55-7.48 (m, 4H), 7.41-7.36 (m, 1H), 7.11 (d, J=2.8 Hz, 1H), 6.99 (dd, J=8.7, 2.8 Hz, 1H), 6.50 (d, J=5.7 Hz, 1H), 6.33 (s, 1H), 3.10-3.01 (m, 1H), 1.25 (s, 9H), 1.12 (d, J=6.8 Hz, 6H).

### Synthesis of Compound (bl): 1-(3-(tert-butyl)-1-phenyl-1H-pyrazol-5-yl)-3-(4-(pyridin-4-yloxy)phenyl)urea

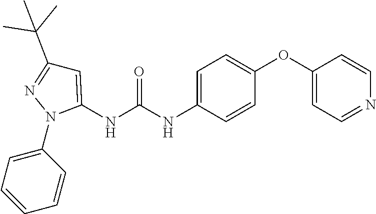 [SMILES: CC(C)(C)c1cc(NC(=O)Nc2ccc(Oc3ccncc3)cc2)n(-c2ccccc2)n1]

### Synthesis of Compound 1.4: 1-(3-(tert-butyl)-1-phenyl-1H-pyrazol-5-yl)-3-(4-(pyridin-4-yloxy)phenyl)urea (Compound (bl))

According to Scheme 4 Step 2: In a sealed tube containing 2,2,2-trichloroethyl N-(5-tert-butyl-2-phenyl-pyrazol-3-yl)carbamate 158 (475 mg, 1.21 mmol, 1.5 eq), was added a solution of 4-(4-pyridyloxy)aniline (150 mg, 0.81 mmol, 1.0 eq), DIEA (1.74 mL, 10.0 mmol) in acetonitrile (0.1M). The reaction mixture was stirred at 70° C. for 3 h. After cooling down, the mixture was directly absorbed on silica gel to be purified by flash chromatography using cyclohexane and ethyl acetate to afford the expected compound as white foam after lyophilisation (Yield: 45%, 156 mg). MS m/z (ES) [M+H]⁺: 428.33

### Synthesis of Compound (bm): 1-(3-(tert-butyl)-1-(quinolin-6-yl)-1H-pyrazol-5-yl)-3-(2-(methylthio)-4-((3-oxo-3,4-dihydropyrido[2,3-b]pyrazin-8-yl)oxy)phenyl)urea

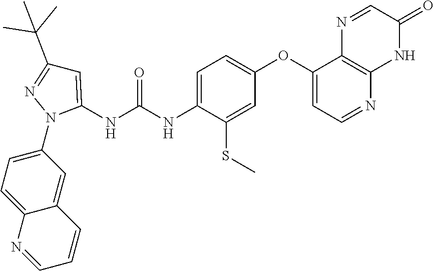 [SMILES: CSc1cc(Oc2ccnc3[nH]c(=O)cnc23)ccc1NC(=O)Nc1cc(C(C)(C)C)nn1-c1ccc2ncccc2c1]

### Step1. Synthesis of Compound 1.10

According to Scheme 3 Step 1: Under Argon, triphosgene (0.148 g, 0.50 mmol) was added portionwise to a solution of 111 (169 mg, 0.50 mmol, 1.0 eq) and Et₃N (0.085 mL, 0.616 mmol) in CH₂Cl₂ (5 mL) at −20° C., the reaction mass was stirred at room temperature for 1 h. Then the solvent was removed under reduced pressure, THF (5 mL) was added to the residue, the formed precipitate was filtered.

### Step 2. Synthesis of: 1-(3-(tert-butyl)-1-(quinolin-6-yl)-1H-pyrazol-5-yl)-3-(2-(methylthio)-4-((3-oxo-3,4-dihydropyrido[2,3-b]pyrazin-8-yl)oxy)phenyl)urea (Compound (bm))

According to Scheme 3 Step 2: Then a solution of 3-(tert-butyl)-1-(quinolin-6-yl)-1H-pyrazol-5-amine 27 (147 mg, 0.55 mmol, 1.1 eq) in CH₂Cl₂ (5 mL) was added dropwise at 0° C. The reaction mixture was stirred at room temperature for 18 h. The residue was purified by semi-preparative HPLC. The fractions were collected and evaporated, and the residue was triturated in dichloromethane and filtered to afford the expected compound as yellow solid (Yield: 4%, 12 mg). MS m/z (ES) [M+H]⁺: 593.33

### Synthesis of Compound (bn): 1-(3-(tert-butyl)-1-cyclohexyl-1H-pyrazol-5-yl)-3-(2-(methylthio)-4-((3-oxo-3,4-dihydropyrido[2,3-b]pyrazin-8-yl)oxy)phenyl)urea

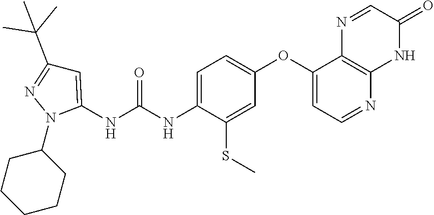 [SMILES: CSc1cc(Oc2ccnc3[nH]c(=O)cnc23)ccc1NC(=O)Nc1cc(C(C)(C)C)nn1C1CCCCC1]

### Step1. Synthesis of Compound 1.10

According to Scheme 3 Step 1: Under Argon, triphosgene (0.130 g, 0.44 mmol) was added portionwise to a solution of 111 (150 mg, 0.44 mmol, 1.0 eq) and Et₃N (0.075 mL, 0.542 mmol) in CH₂Cl₂ (5 mL) at −20° C., the reaction mass was stirred at room temperature for 1 h. Then the solvent was removed under reduced pressure, THF (5 mL) was added to the residue, the formed precipitate was filtered.

### Step 2. Synthesis of: 1-(3-(tert-butyl)-1-cyclohexyl-1H-pyrazol-5-yl)-3-(2-(methylthio)-4-((3-oxo-3,4-dihydropyrido[2,3-b]pyrazin-8-yl)oxy)phenyl)urea (Compound (bn))

According to Scheme 3 Step 2: Then a solution of 5-tert-butyl-2-cyclohexyl-pyrazol-3-amine 180 (107 mg, 0.48 mmol, 1.1 eq) in CH₂Cl₂ (5 mL) was added dropwise at 0° C. The reaction mixture was stirred at room temperature for 18 h. The residue was purified by semi-preparative HPLC. The fractions were collected and evaporated, and the residue was triturated in dichloromethane and filtered to afford the expected compound as yellow solid (Yield: 10%, 23 mg). MS m/z (ES) [M+H]⁺: 548.33

### Synthesis of Compound (bo): 1-(3-(tert-butyl)-1-phenyl-1H-pyrazol-5-yl)-3-(2,3-difluoro-4-((3-oxo-3,4-dihydropyrido[2,3-b]pyrazin-8-yl)oxy)phenyl)urea

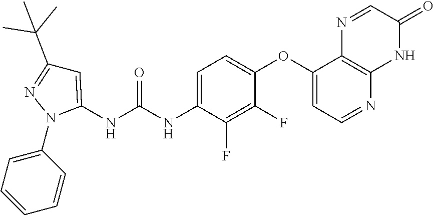 [SMILES: CC(C)(C)c1cc(NC(=O)Nc2ccc(Oc3ccnc4[nH]c(=O)cnc34)c(F)c2F)n(-c2ccccc2)n1]

### Synthesis of Compound 1.4: 1-(3-(tert-butyl)-1-phenyl-1H-pyrazol-5-yl)-3-(2,3-difluoro-4-((3-oxo-3,4-dihydropyrido[2,3-b]pyrazin-8-yl)oxy)phenyl)urea (Compound (bo))

According to Scheme 4 Step 2: In a sealed tube containing 8-(4-amino-2,3-difluoro-phenoxy)-4H-pyrido[2,3-b]pyrazin-3-one hydrochloride 186 (150 mg, 0.46 mmol, 1.0 eq), DIEA (0.48 mL, 2.75 mmol, 6.0 eq) in DMSO (0.1M) was added 2,2,2,-trichloroethyl N-(5-tert-butyl-2-phenyl-pyrazol-3-yl)carbamate 158 (179 mg, 0.46 mmol, 1.0 eq). The reaction mixture was stirred at 70° C. for 20 h. The expected compound was obtained after 2 flash chromatographies (cyclohexane/ethyl acetate then ethyl acetate/methanol) and semi-preparative HPLC as white solid (Yield: 14%, 34 mg). MS m/z (ES) [M+H]⁺: 532.33

### Synthesis of Compound (bp): Tert-Butyl (2-((4-(4-(3-(3-(tert-butyl)-1-phenyl-1H-pyrazol-5-yl)ureido)-3-(methylthio)phenoxy)pyridin-2-yl)amino)-2-oxoethyl)(methyl)carbamate

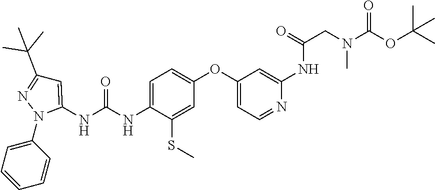 [SMILES: CSc1cc(Oc2ccnc(NC(=O)CN(C)C(=O)OC(C)(C)C)c2)ccc1NC(=O)Nc1cc(C(C)(C)C)nn1-c1ccccc1]

### Synthesis of: Tert-Butyl (2-((4-(4-(3-(3-(tert-butyl)-1-phenyl-1H-pyrazol-5-yl)ureido)-3-(methylthio)phenoxy)pyridin-2-yl)amino)-2-oxoethyl)(methyl)carbamate (Compound (bp))

To a solution of 1-{4-[(2-amino-4-pyridyl)oxy]-2-methylsulfanyl-phenyl}-3-(5-tert-butyl-2-phenyl-pyrazol-3-yl)urea hydrochloride (bj) (84 mg, 0.1 mmol) and Boc-Sat-OH (39 mg, 0.21 mmol) in dry DMF (5 mL) were added DIEA (0.05 mL, 0.27 mmol) followed by HATU (79 mg, 0.21 mmol). The reaction mixture was stirred at room temperature for 48 h. Then sat. aq. NaHCO₃ (40 mL) and AcOEt (40 mL) were added. The aqueous layer was extracted with AcOEt (2×20 mL). The combined organic fractions were dried over MgSO₄, filtered and concentrated in vacuo. The crude obtained was purified by flash chromatography (12 g, SiO₂, cyclohexane/AcOEt 100:0→0:100, 15 CV) to furnish the title compound (Yield: 23%, 25 mg) as a yellow oil. MS m/z (ES) [M+H]⁺: 660.42

¹H NMR (300 MHz, DMSO-d₆) δ 10.57 (d, J=6.7 Hz, 1H), 9.00 (s, 1H), 8.36 (s, 1H), 8.17 (d, J=5.7 Hz, 1H), 7.73 (d, J=8.8 Hz, 1H), 7.62 (s, 1H), 7.53-7.51 (m, 4H), 7.42-7.37 (m, 1H), 7.12 (d, J=2.5 Hz, 1H), 6.95 (dd, J=8.8, 2.7 Hz, 1H), 6.66 (s, 1H), 6.35 (s, 1H), 3.96 (d, J=10.2 Hz, 2H), 2.81 and 2.78 (2s, 3H), 2.40 (s, 3H), 1.37 and 1.27 (2s, 18H).

### Synthesis of Compound (bq): N-(4-(4-(3-(3-(tert-butyl)-1-phenyl-1H-pyrazol-5-yl)ureido)-3-(methylthio)phenoxy)pyridin-2-yl)-2-(methylamino)acetamide dihydrochloride

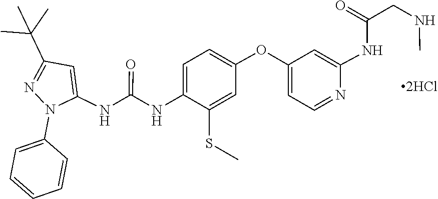 [SMILES: CNCC(=O)Nc1cc(Oc2ccc(NC(=O)Nc3cc(C(C)(C)C)nn3-c3ccccc3)c(SC)c2)ccn1]

### Synthesis of: N-(4-(4-(3-(3-(tert-butyl)-1-phenyl-1H-pyrazol-5-yl)ureido)-3-(methylthio)phenoxy)pyridin-2-yl)-2-(methylamino)acetamide (Compound (bq))

Tert-butyl (2-((4-(4-(3-(3-(tert-butyl)-1-phenyl-1H-pyrazol-5-yl)ureido)-3-(methylthio)phenoxy)pyridin-2-yl)amino)-2-oxoethyl)(methyl)carbamate (bp) (18 mg, 27 μmol) was treated with 4N HCl in dioxane (5 mL) and the reaction mixture was stirred at room temperature for 2 h. The solvent was removed under vacuum leading after lyophilization to the expected product (Yield: 10 mg, 58%) as a brown gum. MS m/z (ES) [M+H]⁺: 560.42

¹H NMR (300 MHz, DMSO-d₆) δ 11.15 (s, 1H), 9.10 (s, 1H), 8.91 (s, 1H), 8.44 (s, 1H), 8.22 (d, J=5.8 Hz, 1H), 7.70 (d, J=8.8 Hz, 1H), 7.55-7.48 (m, 5H), 7.42-7.39 (m, 1H), 7.14 (d, J=2.7 Hz, 1H), 6.97 (dd, J=8.8, 2.6 Hz, 1H), 6.79 (dd, J=5.8, 2.4 Hz, 1H), 6.33 (s, 1H), 3.90 (br s, 2H), 2.55 and 2.53 (2s, 3H), 2.41 (s, 3H), 1.26 (s, 9H).

### Synthesis of Compound (br): 4-(4-(3-(3-(tert-butyl)-1-phenyl-1H-pyrazol-5-yl)ureido)-3-(methylthio)phenoxy)-N-methylpicolinamide

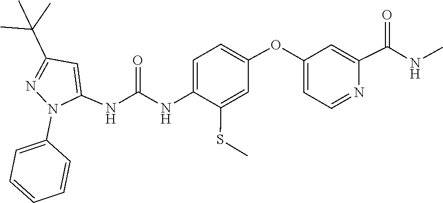 [SMILES: CNC(=O)c1cc(Oc2ccc(NC(=O)Nc3cc(C(C)(C)C)nn3-c3ccccc3)c(SC)c2)ccn1]

### Synthesis of Compound 1.4: 4-(4-(3-(3-(tert-butyl)-1-phenyl-1H-pyrazol-5-yl)ureido)-3-(methylthio)phenoxy)-N-methylpicolinamide (Compound (br))

According to Scheme 4 Step 2: In a sealed tube containing 4-(4-amino-3-methylsulfanyl-phenoxy)-N-methyl-pyridine-2-carboxamide 103 (120 mg, 0.41 mmol), DIEA (0.40 mL, 2.48 mmol) in DMSO (0.1M) was added 2,2,2-trichloroethyl N-(5-tert-butyl-2-phenyl-pyrazol-3-yl)carbamate 158 (242 mg, 0.62 mmol). The reaction mixture was stirred at 70° C. for 18 h to afford after extraction, flash chromatography on silica gel (12 g, SiO₂, cyclohexane/AcOEt 100:0→0:100, 10 CV) the expected compound as a red foam (Yield: 18%, 40 mg). MS m/z (ES) [M+H]⁺: 531.42

¹H NMR (300 MHz, DMSO-d₆) δ 9.01 (s, 1H), 8.76 (qd, J=4.9 Hz, 1H), 8.49 (d, J=5.6 Hz, 1H), 8.38 (s, 1H), 7.76 (d, J=8.8 Hz, 1H), 7.53-7.51 (m, 4H), 7.42-7.37 (m, 2H), 7.17 (d, J=2.7 Hz, 1H), 7.13 (dd, J=5.6, 2.6 Hz, 1H), 7.00 (dd, J=8.8, 2.7 Hz, 1H), 6.35 (s, 1H), 2.77 (d, J=4.8 Hz, 3H), 2.41 (s, 3H), 1.26 (s, 9H).

### Synthesis of Compound (bs): 1-(3-(tert-butyl)-1-phenyl-1H-pyrazol-5-yl)-3-(2-(methylthio)-4-(pyrido[2,3-b]pyrazin-8-yloxy)phenyl)urea

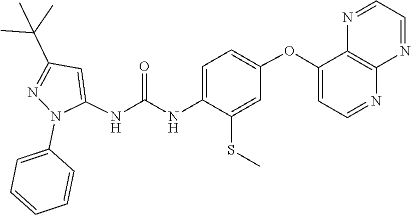 [SMILES: CSc1cc(Oc2ccnc3nccnc23)ccc1NC(=O)Nc1cc(C(C)(C)C)nn1-c1ccccc1]

### Synthesis of Compound 1.4: 1-(3-(tert-butyl)-1-phenyl-1H-pyrazol-5-yl)-3-(2-(methylthio)-4-(pyrido[2,3-b]pyrazin-8-yloxy)phenyl)urea (Compound (bs))

According to Scheme 4 Step 2: In a sealed tube 2-methylsulfanyl-4-pyrido[2,3-b]pyrazin-8-yloxy-aniline hydrochloride 188 (150 mg, 0.47 mmol), DIEA (0.80 mL, 4.68 mmol) in DMSO (0.1M) was added 2,2,2-trichloroethyl N-(5-tert-butyl-2-phenyl-pyrazol-3-yl)carbamate 158 (365 mg, 0.94 mmol). The reaction mixture was stirred at 70° C. for 24 h to afford after extraction, flash chromatography on silica gel (40 g, SiO₂, cyclohexane/AcOEt 100:0→90:10, CV) the expected compound as a beige powder (Yield: 23%, 57 mg). MS m/z (ES) [M+H]⁺: 526.33

¹H NMR (300 MHz, DMSO-d₆) δ 9.17 (d, J=1.7 Hz, 1H), 9.05 (d, J=1.7 Hz, 1H), 9.01 (s, 1H), 8.93 (d, J=5.2 Hz, 1H), 8.41 (s, 1H), 7.78 (d, J=8.8 Hz, 1H), 7.534-7.52 (m, 4H), 7.43-7.37 (m, 1H), 7.27 (d, J=2.6 Hz, 1H), 7.09 (dd, J=8.8, 2.6 Hz, 1H), 6.97 (d, J=5.2 Hz, 1H), 6.36 (s, 1H), 2.42 (s, 3H), 1.26 (s, 9H).

### Synthesis of Compound (bt): 1-(3-(tert-butyl)-1-phenyl-1H-pyrazol-5-yl)-3-(2,5-difluoro-4-((3-oxo-3,4-dihydropyrido[2,3-b]pyrazin-8-yl)oxy)phenyl)urea

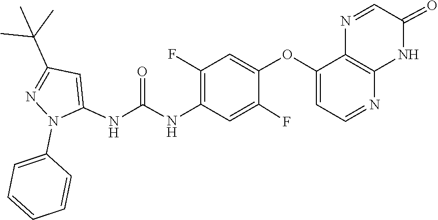 [SMILES: CC(C)(C)c1cc(NC(=O)Nc2cc(F)c(Oc3ccnc4[nH]c(=O)cnc34)cc2F)n(-c2ccccc2)n1]

### Synthesis of Compound 1.4: 1-(3-(tert-butyl)-1-phenyl-1H-pyrazol-5-yl)-3-(2,5-difluoro-4-((3-oxo-3,4-dihydropyrido[2,3-b]pyrazin-8-yl)oxy)phenyl)urea (Compound (bt))

According to Scheme 4 Step 2: In a sealed tube 8-(4-amino-2,5-difluoro-phenoxy)-4H-pyrido[2,3-b]pyrazin-3-one hydrochloride 195 (150 mg, 0.46 mmol, 1.0 eq), DIEA (0.48 mL, 2.75 mmol, 6.0 eq) in DMSO (0.1M) was added 2,2,2,-trichloroethyl N-(5-tert-butyl-2-phenyl-pyrazol-3-yl)carbamate 158 (179 mg, 0.46 mmol, 1.0 eq). The reaction mixture was stirred at 70° C. for 20 h. The expected compound was obtained after 2 flash chromatographies (cyclohexane/ethyl acetate then ethyl acetate/methanol) and semi-preparative HPLC as white solid (Yield: 12%, 28 mg). MS m/z (ES) [M+H]⁺: 532.50

### Synthesis of Compound (bu): 1-(3-(tert-butyl)-1-phenyl-1H-pyrazol-5-yl)-3-(2-(methylthio)-4-((2-oxo-1,2-dihydroquinoxalin-5-yl)oxy)phenyl)urea

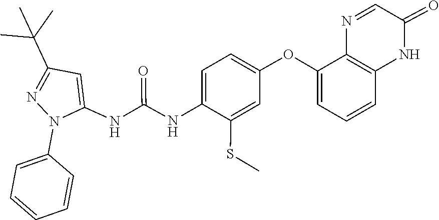 [SMILES: CSc1cc(Oc2cccc3[nH]c(=O)cnc23)ccc1NC(=O)Nc1cc(C(C)(C)C)nn1-c1ccccc1]

### Synthesis of Compound 1.4: 1-(3-(tert-butyl)-1-phenyl-1H-pyrazol-5-yl)-3-(2-(methylthio)-4-((2-oxo-1,2-dihydroquinoxalin-5-yl)oxy)phenyl)urea (Compound (bu))

According to Scheme 4 Step 2: In a sealed tube 5-(4-amino-3-methylsulfanyl-phenoxy)-1H-quinoxalin-2-one 199 (160 mg, 0.53 mmol), DIEA (0.93 ml, 5.3 mmol) in DMSO (0.1M) was added 2,2,2-trichloroethyl N-(5-tert-butyl-2-phenyl-pyrazol-3-yl)carbamate 158 (418 mg, 1.1 mmol). The reaction mixture was stirred at 70° C. for 18 h to afford after extraction, flash chromatography on silica gel (40 g, SiO₂, cyclohexane/AcOEt 100:0→90:10, 10 CV) the expected compound as a yellow solid (Yield: 23%, 66 mg). MS m/z (ES) [M+H]⁺: 541.42

¹H NMR (300 MHz, DMSO-d₆) δ 12.51 (s, 1H), 8.87 (s, 1H), 8.26 (s, 1H), 8.10 (s, 1H), 7.56-7.41 (m, 6H), 7.40-7.35 (m, 1H), 7.06 (d, J=7.4 Hz, 1H), 6.99 (d, J=2.8 Hz, 1H), 6.78-6.70 (m, 2H), 6.32 (s, 1H), 2.37 (s, 3H), 1.25 (s, 9H).

### Synthesis of Compound (by): 1-(3-(tert-butyl)-1-phenyl-1H-pyrazol-5-yl)-3-(2-(methylthio)-4-((7-oxo-7,8-dihydropteridin-4-yl)oxy)phenyl)urea

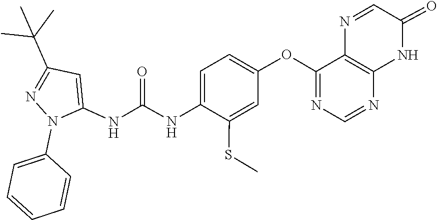 [SMILES: CSc1cc(Oc2ncnc3[nH]c(=O)cnc23)ccc1NC(=O)Nc1cc(C(C)(C)C)nn1-c1ccccc1]

### Synthesis of Compound 1.4: 1-(3-(tert-butyl)-1-phenyl-1H-pyrazol-5-yl)-3-(2-(methylthio)-4-((7-oxo-7,8-dihydropteridin-4-yl)oxy)phenyl)urea (Compound (by))

According to Scheme 4 Step 2: In a sealed tube 4-(4-amino-3-methylsulfanyl-phenoxy)-8H-pteridin-7-one hydrochloride 203 (81 mg, 0.24 mmol), DIEA (0.25 mL, 1.44 mmol) in DMSO (0.1M) was added 2,2,2-trichloroethyl N-(5-tert-butyl-2-phenyl-pyrazol-3-yl)carbamate 158 (94 mg, 0.24 mmol). The reaction mixture was stirred at 80° C. for 18 h to afford after extraction, flash chromatography on silica gel (12 g, SiO₂, cyclohexane/AcOEt 100:0→90:10, 5 CV) the expected compound as a yellow solid (Yield: 31%, 40 mg). MS m/z (ES) [M+H]⁺: 543.42

¹H NMR (300 MHz, DMSO-d₆) δ 13.28 (s, 1H), 8.99 (s, 1H), 8.52 (d, J=3.4 Hz, 1H), 8.38 (s, 1H), 8.23 (s, 1H), 7.71 (d, J=8.8 Hz, 1H), 7.53 (d, J=4.3 Hz, 4H), 7.44-7.38 (m, 1H), 7.22 (d, J=2.6 Hz, 1H), 7.06 (dd, J=8.8, 2.6 Hz, 1H), 6.37 (s, 1H), 2.42 (s, 3H), 1.28 (s, 9H).

### Synthesis of Compound (bw): 1-(3-(tert-butyl)-1-(3-fluorophenyl)-1H-pyrazol-5-yl)-3-(2-(methylthio)-4-((3-oxo-3,4-dihydropyrido[2,3-b]pyrazin-8-yl)oxy)phenyl)urea

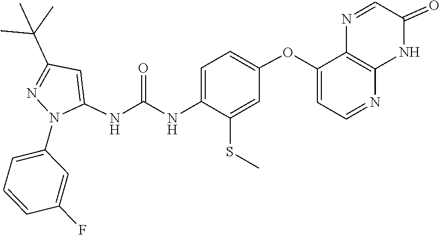 [SMILES: CSc1cc(Oc2ccnc3[nH]c(=O)cnc23)ccc1NC(=O)Nc1cc(C(C)(C)C)nn1-c1cccc(F)c1]

### Step1. Synthesis of Compound 1.10

According to Scheme 3 Step 1: Under Argon, triphosgene (0.130 g, 0.44 mmol) was added portionwise to a solution of 111 (150 mg, 0.44 mmol, 1.0 eq) and Et₃N (0.075 mL, 0.542 mmol) in CH₂Cl₂ (5 mL) at −20° C., the reaction mass was stirred at room temperature for 1 h. Then the solvent was removed under reduced pressure, THF (5 mL) was added to the residue, the formed precipitate was filtered.

### Step 2. Synthesis of Compound 1.4: 1-(3-(tert-butyl)-1-cyclohexyl-1H-pyrazol-5-yl)-3-(2-(methylthio)-4-((3-oxo-3,4-dihydropyrido[2,3-b]pyrazin-8-yl)oxy)phenyl)urea (Compound (bn))

According to Scheme 3 Step 2: Then a solution of 5-tert-butyl-2-(3-fluorophenyl)pyrazol-3-amine hydrochloride (132 mg, 0.48 mmol, 1.1 eq) and triethylamine (0.93 mL, 0.7 mmol, 1.5 eq). in toluene (3 mL) was added dropwise at 0° C. The mixture was stirred in a sealed tube at 80° C. for 20 h, then at 100° C. for 20 h. After cooling down, the solvent was evaporated and the residue was purified by semi-preparative HPLC to afford expected compound as white powder (Yield: 8%, 21 mg). MS m/z (ES) [M+H]⁺: 560.25

### Synthesis of Compound (bx): 1-(3-(tert-butyl)isoxazol-5-yl)-3-(2-(methylthio)-4-((3-oxo-3,4-dihydropyrido[2,3-b]pyrazin-8-yl)oxy)phenyl)urea

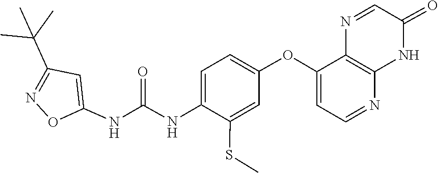 [SMILES: CSc1cc(Oc2ccnc3[nH]c(=O)cnc23)ccc1NC(=O)Nc1cc(C(C)(C)C)no1]

### Step1. Synthesis of Compound 1.10

According to Scheme 3 Step 1: Under Argon, triphosgene (0.130 g, 0.44 mmol) was added portionwise to a solution of 111 (150 mg, 0.44 mmol, 1.0 eq) and Et₃N (0.075 mL, 0.542 mmol) in CH₂Cl₂ (5 mL) at −20° C., the reaction mass was stirred at room temperature for 1 h. Then the solvent was removed under reduced pressure, THF (5 mL) was added to the residue, the formed precipitate was filtered.

### Step 2. Synthesis of Compound 1.4: 1-(3-(tert-butyl)isoxazol-5-yl)-3-(2-(methylthio)-4-((3-oxo-3,4-dihydropyrido[2,3-b]pyrazin-8-yl)oxy)phenyl)urea (Compound (bx))

According to Scheme 3 Step 2: Then a solution of 3-tert-butylisoxazol-5-amine 272 (69 mg, 0.49 mmol, 1.1 eq) in toluene (3 mL) was added dropwise at 0° C. The mixture was stirred in a sealed tube at 80° C. for 20 h. The solid was filtrated, rinsed with ethyl acetate and methanol and purified by semi-preparative HPLC to afford expected compound as white solid (Yield: 9%, 19 mg). MS m/z (ES) [M+H]⁺: 467.25

### Synthesis of Compound (by): 1-(3-(tert-butyl)-1-(p-tolyl)-1H-pyrazol-5-yl)-3-(2-(methylthio)-4-((7-oxo-5,6,7,8-tetrahydro-1,8-naphthyridin-4-yl)oxy)phenyl)urea

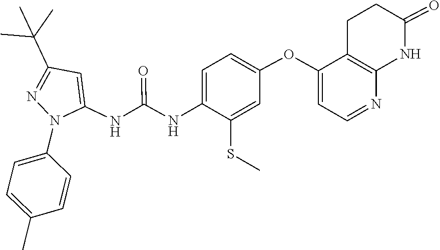 [SMILES: CSc1cc(Oc2ccnc3c2CCC(=O)N3)ccc1NC(=O)Nc1cc(C(C)(C)C)nn1-c1ccc(C)cc1]

### Synthesis of Compound 1.4: 1-(3-(tert-butyl)-1-(p-tolyl)-1H-pyrazol-5-yl)-3-(2-(methylthio)-4-((7-oxo-5,6,7,8-tetrahydro-1,8-naphthyridin-4-yl)oxy)phenyl)urea (Compound (by))

According to Scheme 4 Step 2: In a sealed tube 5-(4-amino-3-methylsulfanyl-phenoxy)-3,4-dihydro-1H-1,8-naphthyridin-2-one 132 (95 mg, 0.31 mmol), DIEA (0.30 mL, 1.89 mmol) in DMSO (0.1M) was added 2,2,2-trichloroethyl N-[5-tert-butyl-2-(p-tolyl)pyrazol-3-yl]carbamate 307 (191 mg, 0.47 mmol). The reaction mixture was stirred at 70° C. for 24 h to afford after filtration and semi-preparative HPLC, the title compound as a white solid (Yield: 21%, 37 mg). MS m/z (ES) [M+H]⁺: 557.42

¹H NMR (300 MHz, DMSO-d₆) δ 10.49 (s, 1H), 8.91 (s, 1H), 8.35 (s, 1H), 7.95 (d, J=5.7 Hz, 1H), 7.70 (d, J=8.8 Hz, 1H), 7.38 (d, J=8.3 Hz, 2H), 7.31 (d, J=8.5 Hz, 2H), 7.09 (d, J=2.6 Hz, 1H), 6.91 (dd, J=8.8, 2.6 Hz, 1H), 6.32 (s, 1H), 6.29 (d, J=5.8 Hz, 1H), 2.90 (t, J=7.4 Hz, 2H), 2.51 (t, J=7.4 Hz, 2H), 2.40 (s, 3H), 2.35 (s, 3H), 1.25 (s, 9H).

### Synthesis of Compound (bz): 1-(3-(tert-butyl)-1-(3-chlorophenyl)-1H-pyrazol-5-yl)-3-(2-(methylthio)-4-((7-oxo-5,6,7,8-tetrahydro-1,8-naphthyridin-4-yl)oxy)phenyl)urea

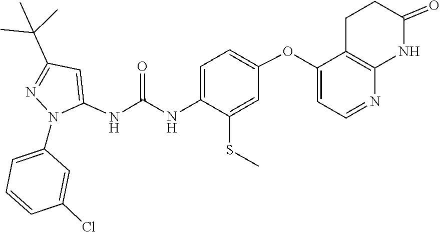 [SMILES: CSc1cc(Oc2ccnc3c2CCC(=O)N3)ccc1NC(=O)Nc1cc(C(C)(C)C)nn1-c1cccc(Cl)c1]

### Step1. Synthesis of Compound 1.11

According to Scheme 4 Step 1: At 0° C., to a solution of N-[5-tert-butyl-2-(3-chlorophenyl)pyrazol-3-yl]amine (500 mg, 2.0 mmol, 1.0 eq) in dry THF (0.2M) was added dropwise dry pyridine (1.1 eq) followed by 2,2,2-trichloroethylchloroformate (0.31 mL, 2.2 mmol, 1.1 eq). The reaction mixture was stirred at room temperature for 18 h. Water was added, and the reaction mixture was extracted with ethyl acetate. The organic layers were combined, dried over magnesium sulfate, filtered and evaporated. The 2,2,2-trichloroethyl N-[5-tert-butyl-2-(3-chlorophenyl)pyrazol-3-yl]carbamate was obtained as a brown gum (0.927 g, quantitative yield) and was used without purification in the next step.

### Step2. Synthesis of Compound 1.4: 1-(3-(tert-butyl)-1-(3-chlorophenyl)-1H-pyrazol-5-yl)-3-(2-(methylthio)-4-((7-oxo-5,6,7,8-tetrahydro-1,8-naphthyridin-4-yl)oxy)phenyl)urea (Compound (bz))

According to Scheme 4 Step 2: In a sealed tube containing 5-(4-amino-3-methylsulfanyl-phenoxy)-3,4-dihydro-1H-1,8-naphthyridin-2-one 132 (120 mg, 0.40 mmol), DIEA (0.40 mL, 2.40 mmol) in DMSO (0.1M) was added 2,2,2-trichloroethyl N-[5-tert-butyl-2-(3-chlorophenyl)pyrazol-3-yl]carbamate (245 mg, 0.60 mmol). The reaction mixture was stirred at 70° C. for 18 h to furnish after filtration the title compound (Yield: 67%, 155 mg) as a white solid. MS m/z (ES) [M+H]⁺: 577.33

¹H NMR (300 MHz, DMSO-d₆) δ 10.49 (s, 1H), 8.99 (s, 1H), 8.33 (s, 1H), 7.95 (d, J=5.8 Hz, 1H), 7.65 (d, J=8.8 Hz, 1H), 7.60 (s, 1H), 7.54-7.52 (m, 2H), 7.48-7.42 (m, 1H), 7.09 (d, J=2.7 Hz, 1H), 6.88 (dd, J=8.8, 2.6 Hz, 1H), 6.35 (s, 1H), 6.29 (d, J=5.7 Hz, 1H), 2.90 (t, J=8.0 Hz, 2H), 2.51 (t, J=8.0 Hz, 2H), 2.41 (s, 3H), 1.26 (s, 9H).

### Synthesis of Compound (ca): 1-(4-(tert-butyl)thiazol-2-yl)-3-(2-(methylthio)-4-((3-oxo-3,4-dihydropyrido[2,3-b]pyrazin-8-yl)oxy)phenyl)urea

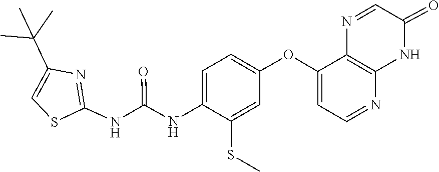 [SMILES: CSc1cc(Oc2ccnc3[nH]c(=O)cnc23)ccc1NC(=O)Nc1nc(C(C)(C)C)cs1]

### Step1. Synthesis of Compound 1.10

According to Scheme 3 Step 1: Under Argon, triphosgene (0.130 g, 0.44 mmol) was added portionwise to a solution of 111 (150 mg, 0.44 mmol, 1.0 eq) and Et₃N (0.075 mL, 0.542 mmol) in CH₂Cl₂ (5 mL) at −20° C., the reaction mass was stirred at room temperature for 1 h. Then the solvent was removed under reduced pressure, THF (5 mL) was added to the residue, the formed precipitate was filtered.

### Step 2. Synthesis of Compound 1.4: 1-(4-(tert-butyl)thiazol-2-yl)-3-(2-(methylthio)-4-((3-oxo-3,4-dihydropyrido[2,3-b]pyrazin-8-yl)oxy)phenyl)urea (Compound (ca))

According to Scheme 3 Step 2: Then a solution 4-tert-butylthiazol-2-amine 156 (77 mg, 0.49 mmol, 1.1 eq) in toluene (3 mL) was added dropwise at 0° C. The mixture was stirred in a sealed tube at 80° C. for 20 h. The solid was filtrated, rinsed with ethyl acetate and methanol and purified by semi-preparative HPLC to afford expected compound as beige solid (Yield: 28%, 61 mg). MS m/z (ES) [M+H]⁺: 483.33

### Synthesis of Compound (cb): 1-(5-(tert-butyl)thiazol-2-yl)-3-(2-(methylthio)-4-((7-oxo-5,6,7,8-tetrahydro-1,8-naphthyridin-4-yl)oxy)phenyl)urea

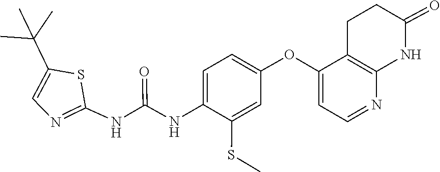 [SMILES: CSc1cc(Oc2ccnc3c2CCC(=O)N3)ccc1NC(=O)Nc1ncc(C(C)(C)C)s1]

### Step2. Synthesis of Compound 1.4: 1-(5-(tert-butyl)thiazol-2-yl)-3-(2-(methylthio)-4-((7-oxo-5,6,7,8-tetrahydro-1,8-naphthyridin-4-yl)oxy)phenyl)urea (Compound (cb))

According to Scheme 4 Step 2: In a sealed tube containing 5-(4-amino-3-methylsulfanyl-phenoxy)-3,4-dihydro-1H-1,8-naphthyridin-2-one 132 (95 mg, 0.31 mmol), DIEA (0.30 mL, 1.90 mmol) in DMSO (0.1M) was added 2,2,2-trichloroethyl N-(5-tert-butylthiazol-2-yl)carbamate 157 (160 mg, 0.47 mmol). The reaction mixture was stirred at 70° C. for 24 h to furnish after filtration the title compound (Yield: 76%, 115 mg) as a beige powder. MS m/z (ES) [M+H]⁺: 484.33

¹H NMR (300 MHz, DMSO-d₆) δ 10.91 (br s, 1H), 10.49 (s, 1H), 8.58 (br s, 1H), 7.96 (d, J=5.8 Hz, 1H), 7.84 (d, J=8.8 Hz, 1H), 7.14 (d, J=2.7 Hz, 1H), 7.05 (s, 1H), 6.96 (dd, J=8.8, 2.7 Hz, 1H), 6.32 (d, J=5.8 Hz, 1H), 2.91 (t, J=7.4 Hz, 2H), 2.51 (t, J=8.1 Hz, 2H), 2.44 (s, 3H), 1.29 (s, 9H).

### Synthesis of Compound (cc): 1-(3-(tert-butyl)-1-(4-cyanophenyl)-1H-pyrazol-5-yl)-3-(2-(methylthio)-4-((3-oxo-3,4-dihydropyrido[2,3-b]pyrazin-8-yl)oxy)phenyl)urea

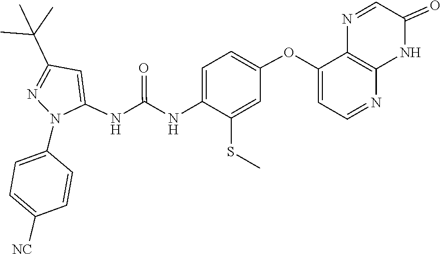 [SMILES: CSc1cc(Oc2ccnc3[nH]c(=O)cnc23)ccc1NC(=O)Nc1cc(C(C)(C)C)nn1-c1ccc(C#N)cc1]

### Step2. Synthesis of Compound 1.4: 1-(3-(tert-butyl)-1-(4-cyanophenyl)-1H-pyrazol-5-yl)-3-(2-(methylthio)-4-((3-oxo-3,4-dihydropyrido[2,3-b]pyrazin-8-yl)oxy)phenyl)urea (Compound (cc))

According to Scheme 4 Step 2: In a sealed tube containing 8-(4-amino-3-(methylthio)phenoxy)pyrido[2,3-b]pyrazin-3(4H)-one, hydrochloride 111 (200 mg, 0.59 mmol, 1.0 eq) DIEA (6 eq) in DMSO (0.1M) was added 2,2,2-trichloroethyl N-[5-tert-butyl-2-(4-cyanophenyl)pyrazol-3-yl]carbamate 206 (490 mg, 1.18 mmol, 2.0 eq). The reaction mixture was stirred at 70° C. for 18 h The residue was evaporated and purified by semi-preparative HPLC followed by trituration in methanol to afford expected compound as orange solid (Yield: 10%, 33 mg). MS m/z (ES) [M+H]⁺: 567.42

### Synthesis of Compound (cd): 1-(3-(tert-butyl)-1-(3,4-dimethylphenyl)-1H-pyrazol-5-yl)-3-(2-(methylthio)-4-((3-oxo-3,4-dihydropyrido[2,3-b]pyrazin-8-yl)oxy)phenyl)urea

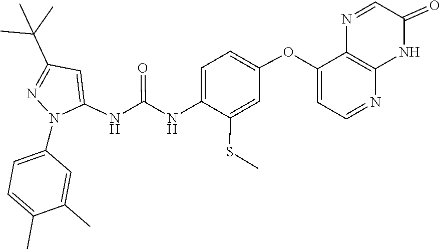 [SMILES: CSc1cc(Oc2ccnc3[nH]c(=O)cnc23)ccc1NC(=O)Nc1cc(C(C)(C)C)nn1-c1ccc(C)c(C)c1]

### Step2. Synthesis of Compound 1.4: 1-(3-(tert-butyl)-1-(3,4-dimethylphenyl)-1H-pyrazol-5-yl)-3-(2-(methylthio)-4-((3-oxo-3,4-dihydropyrido[2,3-b]pyrazin-8-yl)oxy)phenyl)urea (Compound (cd))

According to Scheme 4 Step 2: In a sealed tube containing 8-(4-amino-3-(methylthio)phenoxy)pyrido[2,3-b]pyrazin-3(4H)-one, hydrochloride 111 (150 mg, 0.45 mmol, 1.0 eq), DIEA (6 eq) in DMSO (0.1M) was added 2,2,2-trichloroethyl N-[5-tert-butyl-2-(3,4-dimethylphenyl)pyrazol-3-yl]carbamate 209 (373 mg, 0.89 mmol, 2.0 eq). The reaction mixture was stirred at 70° C. for 18 h. The residue was evaporated and purified by 2 flash chromatographies using cyclohexane and ethyl acetate and then ethyl acetate and methanol. A semi-preparative HPLC was the performed to obtain the expected compound as beige powder (Yield: 7%, 18 mg). MS m/z (ES) [M+H]⁺: 570.50

### Synthesis of Compound (ce): 1-(3-(tert-butyl)-1-(4-(2-(piperidin-1-yl)ethoxy)phenyl)-1H-pyrazol-5-yl)-3-(2-(methylthio)-4-((3-oxo-3,4-dihydropyrido[2,3-b]pyrazin-8-yl)oxy)phenyl)urea

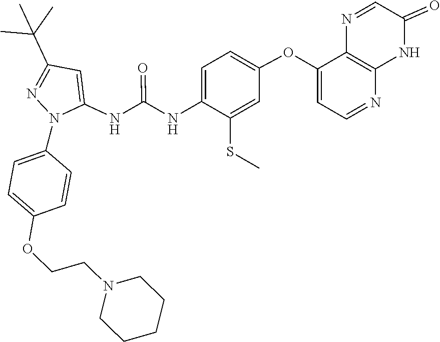 [SMILES: CSc1cc(Oc2ccnc3[nH]c(=O)cnc23)ccc1NC(=O)Nc1cc(C(C)(C)C)nn1-c1ccc(OCCN2CCCCC2)cc1]

### Synthesis of Compound 1.4: 1-(3-(tert-butyl)-1-(4-(2-(piperidin-1-yl)ethoxy)phenyl)-1H-pyrazol-5-yl)-3-(2-(methylthio)-4-((3-oxo-3,4-dihydropyrido[2,3-b]pyrazin-8-yl)oxy)phenyl)urea (Compound (ce))

According to Scheme 4 Step 2: In a sealed tube containing 8-(4-amino-3-(methylthio)phenoxy)pyrido[2,3-b]pyrazin-3(4H)-one, hydrochloride 111 (265 mg, 0.79 mmol, 1.0 eq), DIEA (6 eq) in DMSO (0.1M) was added 2,2,2-trichloroethyl N-[5-tert-butyl-2-[4-[2-(1-piperidyl)ethoxy]phenyl]pyrazol-3-yl]carbamate 211 (612 mg, 1.18 mmol, 1.5 eq). The reaction mixture was stirred at 70° C. for 18 h. The reaction mixture was evaporated, and the residue was purified by flash chromatography over silica gel using cyclohexane and ethyl acetate and then ethyl acetate and methanol. The compound obtained was purified by semi-preparative HPLC to afford, after lyophilization, the expected compound as yellow foam (Yield: 3%, 13 mg). MS m/z (ES) [M+H]⁺: 669.33

### Synthesis of Compound (cf): 1-(3-(tert-butyl)-1-(4-(2-morpholinoethoxy)phenyl)-1H-pyrazol-5-yl)-3-(2-(methylthio)-4-((3-oxo-3,4-dihydropyrido[2,3-b]pyrazin-8-yl)oxy)phenyl)urea

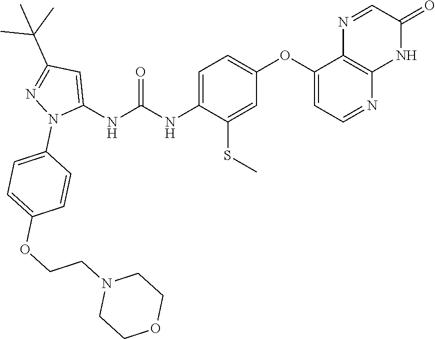 [SMILES: CSc1cc(Oc2ccnc3[nH]c(=O)cnc23)ccc1NC(=O)Nc1cc(C(C)(C)C)nn1-c1ccc(OCCN2CCOCC2)cc1]

### Synthesis of Compound 1.4: 1-(3-(tert-butyl)-1-(4-(2-morpholinoethoxy)phenyl)-1H-pyrazol-5-yl)-3-(2-(methylthio)-4-((3-oxo-3,4-dihydropyrido[2,3-b]pyrazin-8-yl)oxy)phenyl)urea (Compound (cf))

According to Scheme 4 Step 2: In a sealed tube containing 8-(4-amino-3-(methylthio)phenoxy)pyrido[2,3-b]pyrazin-3(4H)-one, hydrochloride 111 (262 mg, 0.78 mmol, 1.0 eq), DIEA (6 eq) in DMSO (0.1M) was added 2,2,2-trichloroethyl N-[5-tert-butyl-2-[4-[2-morpholino-ethoxy]phenyl]pyrazol-3-yl]carbamate 213 (606 mg, 1.16 mmol, 1.5 eq). The reaction mixture was stirred at 70° C. for 18 h. The reaction mixture was evaporated, and the residue was purified by flash chromatography over silica gel using cyclohexane and ethyl acetate and then ethyl acetate and methanol. The compound obtained was purified by semi-preparative HPLC to afford, after lyophilization, the expected compound as yellow solid (Yield: 2%, 11 mg). MS m/z (ES) [M+H]⁺: 671.50

### Synthesis of Compound (cg): 1-(2-(methylthio)-4-((7-oxo-5,6,7,8-tetrahydro-1,8-naphthyridin-4-yl)oxy)phenyl)-3-(5-(trifluoromethyl)pyridin-3-yl)urea

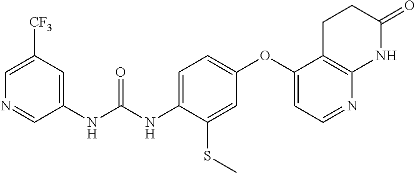 [SMILES: CSc1cc(Oc2ccnc3c2CCC(=O)N3)ccc1NC(=O)Nc1cncc(C(F)(F)F)c1]

### Step1. Synthesis of Compound 1.11

According to Scheme 4 Step 1: At 0° C., to a solution of 5-(trichloromethyl)pyridin-3-amine (423 mg, 2.00 mmol, 1.0 eq) in dry THF (0.2M) was added dropwise dry pyridine (1.1 eq) followed by 2,2,2-trichloroethylchloroformate (0.31 mL, 2.2 mmol, 1.1 eq). The reaction mixture was stirred at room temperature for 18 h. Water was added, and the reaction mixture was extracted with ethyl acetate. The organic layers were combined, dried over magnesium sulfate, filtered and evaporated. The 2,2,2-trichloroethyl (5-(trifluoromethyl)pyridin-3-yl)carbamate was obtained as a brown gum (0.772 g, quantitative yield) and was used without purification in the next step.

### Step2. Synthesis of Compound 1.4: 1-(2-(methylthio)-4-((7-oxo-5,6,7,8-tetrahydro-1,8-naphthyridin-4-yl)oxy)phenyl)-3-(5-(trifluoromethyl)pyridin-3-yl)urea (Compound (cg))

According to Scheme 4 Step 2: In a sealed tube containing 5-(4-amino-3-methylsulfanyl-phenoxy)-3,4-dihydro-1H-1,8-naphthyridin-2-one 132 (85 mg, 0.28 mmol), DIEA (0.30 mL, 1.90 mmol) in DMSO (0.1M) was added 2,2,2-trichloroethyl N-[5-(trifluoromethyl)-3-pyridyl]carbamate (160 mg, 0.47 mmol). The reaction mixture was stirred at 70° C. for 18 h to furnish after flash chromatography (12 g, SiO₂, cyclohexane/AcOEt 100:0→0:100, 10 CV and AcOEt/CH₃OH 100:0→80:20, 5 CV) and semi-preparative HPLC, the title compound (Yield: 5%, 7 mg) as a white solid. MS m/z (ES) [M+H]⁺: 490.25

¹H NMR (300 MHz, DMSO-d₆) δ 10.50 (s, 1H), 9.86 (s, 1H), 8.74 (d, J=2.2 Hz, 1H), 8.55 (s, 1H), 8.43 (s, 1H), 8.37 (s, 1H), 7.97 (d, J=5.8 Hz, 1H), 7.77 (d, J=8.8 Hz, 1H), 7.14 (d, J=2.7 Hz, 1H), 6.95 (dd, J=8.8, 2.7 Hz, 1H), 6.31 (d, J=5.7 Hz, 1H), 2.91 (t, J=7.7 Hz, 2H), 2.51 (t, J=8.1 Hz, 2H), 2.44 (s, 3H).

### Synthesis of Compound (ch): Tert-Butyl (4-(4-(3-(3-(tert-butyl)-1-phenyl-1H-pyrazol-5-yl)ureido)-3-(methylthio)phenoxy)pyridin-2-yl)(2-(dimethylamino)ethyl)carbamate

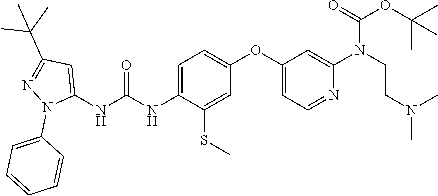 [SMILES: CSc1cc(Oc2ccnc(N(CCN(C)C)C(=O)OC(C)(C)C)c2)ccc1NC(=O)Nc1cc(C(C)(C)C)nn1-c1ccccc1]

### Synthesis of: Tert-Butyl (4-(4-(3-(3-(tert-butyl)-1-phenyl-1H-pyrazol-5-yl)ureido)-3-(methylthio)phenoxy)pyridin-2-yl)(2-(dimethylamino)ethyl)carbamate (Compound (ch))

A mixture of 2-dimethylaminoethylchloride hydrochloride (98 mg, 0.68 mmol) and triethylamine (0.14 mL, 1.02 mmol) in dry DMF (2.5 mL) was added to a solution of tert-butyl N-[4-{4-[(5-tert-butyl-2-phenyl-pyrazol-3-yl)carbamoylamino]-3-methylsulfanyl-phenoxy}-2-pyridyl]carbamate (bi) (100 mg, 0.17 mmol) and NaH (41 mg, 1.02 mmol) in dry DMF (2.5 mL). The reaction mixture was stirred at room temperature for 20 h. Then water (20 mL) and AcOEt (40 mL) were added. The aqueous layer was extracted with AcOEt (2×20 mL). The combined organic fractions were dried over MgSO₄, filtered and concentrated in vacuo. The crude obtained was purified by flash chromatography (12 g, SiO₂, cyclohexane/AcOEt 100:0→0:100, 10 CV and AcOEt/CH₃OH 100:0→85:15, 5 CV) to furnish the title compound (Yield: 39%, 44 mg) as a white solid. MS m/z (ES) [M+H]⁺: 660.42

¹H NMR (300 MHz, DMSO-d₆) δ 8.99 (s, 1H), 8.38 (s, 1H), 8.25 (d, J=5.7 Hz, 1H), 7.75 (d, J=8.8 Hz, 1H), 7.54-7.52 (m, 4H), 7.44-7.39 (m, 1H), 7.12 (dd, J=8.4, 2.7 Hz, 2H), 6.97 (dd, J=8.8, 2.7 Hz, 1H), 6.73 (dd, J=5.7, 2.3 Hz, 1H), 6.36 (s, 1H), 3.92 (t, J=6.9 Hz, 2H), 2.43 (s, 3H), 2.41-2.39 (m, 2H), 2.13 (s, 6H), 1.38 (s, 9H), 1.28 (s, 9H).

### Synthesis of Compound (ci): 1-(3-(tert-butyl)-1-phenyl-1H-pyrazol-5-yl)-3-(2-(methylthio)-4-((2-oxo-1,2,3,4-tetrahydroquinolin-5-yl)oxy)phenyl)urea

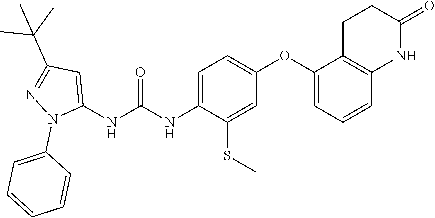 [SMILES: CSc1cc(Oc2cccc3c2CCC(=O)N3)ccc1NC(=O)Nc1cc(C(C)(C)C)nn1-c1ccccc1]

### Synthesis of Compound 1.4: 1-(3-(tert-butyl)-1-phenyl-1H-pyrazol-5-yl)-3-(2-(methylthio)-4-((2-oxo-1,2,3,4-tetrahydroquinolin-5-yl)oxy)phenyl)urea (Compound (ci))

According to Scheme 4 Step 2: In a sealed tube containing 5-(4-amino-3-methylsulfanyl-phenoxy)-3,4-dihydro-1H-quinolin-2-one 132 (214 mg, 0.71 mmol), DIEA (0.37 mL, 2.14 mmol), in DMSO (0.1M) was added 2,2,2-trichloroethyl N-(5-tert-butyl-2-phenyl-pyrazol-3-yl)carbamate 158 (278 mg, 0.71 mmol). The reaction mixture was stirred at 80° C. for 18 h to furnish, after filtration and semi-preparative HPLC, the title compound (Yield: 18%, 68 mg) as a white solid. MS m/z (ES) [M+H]⁺: 542.33

¹H NMR (300 MHz, DMSO-d₆) δ 10.22 (s, 1H), 8.87 (s, 1H), 8.26 (s, 1H), 7.56 (d, J=8.8 Hz, 1H), 7.53-7.51 (m, 4H), 7.43-7.38 (m, 1H), 7.13 (t, J=8.1 Hz, 1H), 6.93 (d, J=2.7 Hz, 1H), 6.70-6.67 (m, 2H), 6.49 (d, J=7.5 Hz, 1H), 6.34 (s, 1H), 2.81 (t, J=7.1 Hz, 2H), 2.43 (t, J=7.1 Hz, 2H), 2.38 (s, 3H), 1.27 (s, 9H).

### Synthesis of Compound (cj): 1-(5-(tert-butyl)-1,3,4-oxadiazol-2-yl)-3-(2-(methylthio)-4-((3-oxo-3,4-dihydropyrido[2,3-b]pyrazin-8-yl)oxy)phenyl)urea

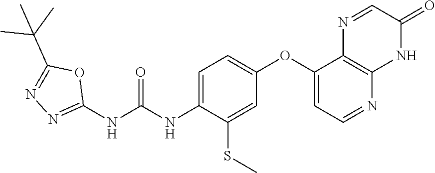 [SMILES: CSc1cc(Oc2ccnc3[nH]c(=O)cnc23)ccc1NC(=O)Nc1nnc(C(C)(C)C)o1]

### Step1. Synthesis of Compound 1.10

According to Scheme 3 Step 1: Under Argon, triphosgene (0.130 g, 0.44 mmol) was added portionwise to a solution of 111 (150 mg, 0.44 mmol, 1.0 eq) and Et₃N (0.075 mL, 0.542 mmol) in CH₂Cl₂ (5 mL) at −20° C., the reaction mass was stirred at room temperature for 1 h. Then the solvent was removed under reduced pressure, THF (5 mL) was added to the residue, the formed precipitate was filtered.

### Step 2. Synthesis of Compound 1.4: 1-(5-(tert-butyl)-1,3,4-oxadiazol-2-yl)-3-(2-(methylthio)-4-((3-oxo-3,4-dihydropyrido[2,3-b]pyrazin-8-yl)oxy)phenyl)urea (Compound (cj))

According to Scheme 3 Step 2: Then a solution 5-tert-1,3,4-oxadiazol-2-amine (69 mg, 0.49 mmol, 1.1 eq) in toluene (3 mL) was added dropwise at 0° C. The mixture was stirred in a sealed tube at 100° C. for 2 days. The solid was filtrated, rinsed with diethyl ether and purified by flash chromatography on silica gel using cyclohexane and ethyl acetate and then ethyl acetate and methanol. The expected compound was obtained as white solid (Yield: 10%, 23 mg). MS m/z (ES) [M+H]⁺: 468.33

### Synthesis of Compound (ck): 1-(3-(tert-butyl)-1-phenyl-1H-pyrazol-5-yl)-3-(4-((2-((2-(dimethylamino)ethyl)amino)pyridin-4-yl)oxy)-2-(methylthio)phenyl)urea Hydrochloride

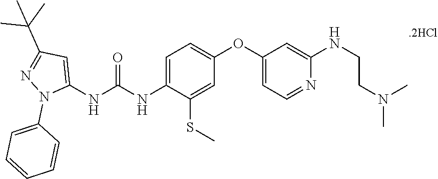 [SMILES: CSc1cc(Oc2ccnc(NCCN(C)C)c2)ccc1NC(=O)Nc1cc(C(C)(C)C)nn1-c1ccccc1]

### Synthesis of: 1-(3-(tert-butyl)-1-phenyl-1H-pyrazol-5-yl)-3-(4-((2-((2-(dimethylamino)ethyl)amino)pyridin-4-yl)oxy)-2-(methylthio)phenyl)urea dihydrochloride (Compound (ck))

tert-butyl N-[4-{4-[(5-tert-butyl-2-phenyl-pyrazol-3-yl)carbamoyl amino]-3-methylsulfanyl-phenoxy}-2-pyridyl]-N-[2-(dimethylamino)ethyl]carbamate (ch) (20 mg, 32 μmol) was treated with 4N HCl in dioxane (3 mL) and the reaction mixture was stirred at room temperature for 3 h. Then the solvent was removed under vacuum leading after lyophilization to the expected product (Yield: 88%, 1 mg) as a beige foam. MS m/z (ES) [M+H]⁺: 560.42

¹H NMR (300 MHz, DMSO-d₆) δ 10.17 (br s, 1H), 9.10 (s, 1H), 8.47 (s, 1H), 7.97 (d, J=7.1 Hz, 1H), 7.78 (d, J=8.8 Hz, 1H), 7.54-7.53 (m, 4H), 7.43-7.39 (m, 1H), 7.19 (d, J=8.4, 2.6 Hz, 1H), 7.04 (d, J=8.8 Hz, 1H), 6.66 (br s, 1H), 6.35 (s, 1H), 6.23 (br s, 1H), 3.78-3.73 (m, 2H), 3.29-3.24 (m, 2H), 2.80 (s, 6H), 2.45 (s, 3H), 1.28 (s, 9H).

### Synthesis of Compound (cl): 1-(3-(tert-butyl)-1-(3-chloro-4-methylphenyl)-1H-pyrazol-5-yl)-3-(2-(methylthio)-4-((3-oxo-3,4-dihydropyrido[2,3-b]pyrazin-8-yl)oxy)phenyl)urea

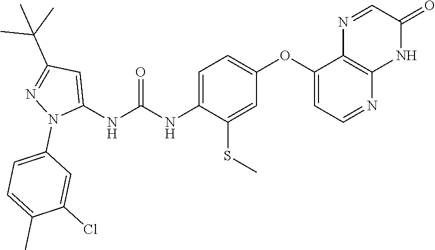 [SMILES: CSc1cc(Oc2ccnc3[nH]c(=O)cnc23)ccc1NC(=O)Nc1cc(C(C)(C)C)nn1-c1ccc(C)c(Cl)c1]

### Synthesis of Compound 1.4: 1-(3-(tert-butyl)-1-(3-chloro-4-methylphenyl)-1H-pyrazol-5-yl)-3-(2-(methylthio)-4-((3-oxo-3,4-dihydropyrido[2,3-b]pyrazin-8-yl)oxy)phenyl)urea (Compound (cl))

According to Scheme 4 Step 2: In a sealed tube containing 8-(4-amino-3-(methylthio)phenoxy)pyrido[2,3-b]pyrazin-3(4H)-one, hydrochloride 111 (150 mg, 0.45 mmol, 1.0 eq), DIEA (6 eq) in DMSO (0.1M) was added 2,2,2-trichloroethyl N-[5-tert-butyl-2-(3-chloro-4-methyl-phenyl)pyrazol-3-yl]carbamate 216 (391 mg, 0.89 mmol, 2.0 eq). The reaction mixture was stirred at 70° C. for 18 h. The residue was evaporated and purified by semi-preparative HPLC. The fractions were evaporated and triturated twice in methanol to afford the expected compound as beige powder (Yield: 8%, 21 mg). MS m/z (ES) [M+H]⁺: 590.42

### Synthesis of Compound (cm): 1-(3-(tert-butyl)-1-(3-chloro-4-fluorophenyl)-1H-pyrazol-5-yl)-3-(2-(methylthio)-4-((3-oxo-3,4-dihydropyrido[2,3-b]pyrazin-8-yl)oxy)phenyl)urea

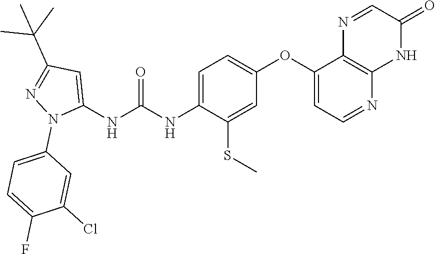 [SMILES: CSc1cc(Oc2ccnc3[nH]c(=O)cnc23)ccc1NC(=O)Nc1cc(C(C)(C)C)nn1-c1ccc(F)c(Cl)c1]

### Synthesis of Compound 1.4: 1-(3-(tert-butyl)-1-(3-chloro-4-methylphenyl)-1H-pyrazol-5-yl)-3-(2-(methylthio)-4-((3-oxo-3,4-dihydropyrido[2,3-b]pyrazin-8-yl)oxy)phenyl)urea (Compound (cl))

According to Scheme 4 Step 2: In a sealed tube containing 8-(4-amino-3-(methylthio)phenoxy)pyrido[2,3-b]pyrazin-3(4H)-one, hydrochloride 111 (150 mg, 0.45 mmol, 1.0 eq), DIEA (6 eq) in DMSO (0.1M) was added 2,2,2-trichloroethyl N-[5-tert-butyl-2-(3-chloro-4-fluoro-phenyl)pyrazol-3-yl]carbamate 219 (395 mg, 0.89 mmol, 2.0 eq). The reaction mixture was stirred at 70° C. for 18 h. The reaction mixture was evaporated and triturated in hot methanol. The solid obtained was filtered and purified by semi-preparative HPLC. The fractions were evaporated and triturated twice in methanol to afford the expected compound as beige powder (Yield: 5%, 14 mg). MS m/z (ES) [M+H]⁺: 594.42

### Synthesis of Compound (cn): 1-(4-((1H-indazol-4-yl)oxy)-2-(methylthio)phenyl)-3-(3-(tert-butyl)-1-phenyl-1H-pyrazol-5-yl)urea

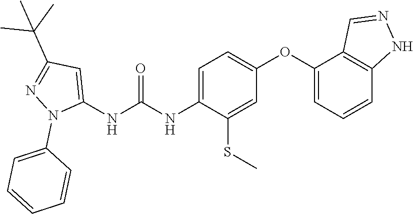 [SMILES: CSc1cc(Oc2cccc3[nH]ncc23)ccc1NC(=O)Nc1cc(C(C)(C)C)nn1-c1ccccc1]

### Synthesis of Compound 1.4: 1-(4-((1H-indazol-4-yl)oxy)-2-(methylthio)phenyl)-3-(3-(tert-butyl)-1-phenyl-1H-pyrazol-5-yl)urea (Compound (cn))

According to Scheme 4 Step 2: In a sealed tube containing 4-(1H-indazol-4-yloxy)-2-methylsulfanyl-aniline 222 (80 mg, 0.29 mmol), DIEA (0.30 mL, 1.77 mmol) in DMSO (0.1M) was added 2,2,2-trichloroethyl N-(5-tert-butyl-2-phenyl-pyrazol-3-yl)carbamate 158 (172 mg, 0.44 mmol). The reaction mixture was stirred at 70° C. for 18 h to furnish after flash chromatography (12 g, SiO₂, cyclohexane/AcOEt 100:0→0:100, 10 CV) and semi-preparative HPLC, the title compound (Yield: 6%, 8 mg) as a white solid. MS m/z (ES) [M+H]⁺: 513.40

¹H NMR (300 MHz, DMSO-d₆) δ 13.21 (s, 1H), 8.93 (s, 1H), 8.32 (s, 1H), 7.85 (s, 1H), 7.52-7.50 (m, 4H), 7.42-7.36 (m, 1H), 7.25 (d, J=4.5 Hz, 2H), 7.10 (d, J=2.7 Hz, 1H), 6.87 (dd, J=8.8, 2.7 Hz, 1H), 6.46 (t, J=4.1 Hz, 1H), 6.34 (s, 1H), 2.38 (s, 3H), 1.25 (s, 9H).

### Synthesis of Compound (co): 4-(4-(3-(3-(tert-butyl)-1-phenyl-1H-pyrazol-5-yl)ureido)-3-(methylthio)phenoxy)-N-phenylpicolinamide

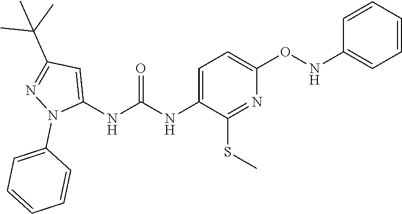 [SMILES: CSc1nc(ONc2ccccc2)ccc1NC(=O)Nc1cc(C(C)(C)C)nn1-c1ccccc1]

### Synthesis of Compound 1.4: 4-(4-(3-(3-(tert-butyl)-1-phenyl-1H-pyrazol-5-yl)ureido)-3-(methylthio)phenoxy)-N-phenylpicolinamide (Compound (co))

According to Scheme 4 Step 2: In a sealed tube containing 4-(4-amino-3-methylsulfanyl-phenoxy)-N-phenyl-pyridine-2-carboxamide 224 (200 mg, 0.57 mmol), DIEA (0.60 mL, 3.41 mmol) in DMSO (0.1M) was added 2,2,2-trichloroethyl N-(5-tert-butyl-2-phenyl-pyrazol-3-yl)carbamate 158 (333 mg, 0.85 mmol). The reaction mixture was stirred at 70° C. for 24 h to furnish after flash chromatography (12 g, SiO₂, cyclohexane/AcOEt 100:0→70:30, 10 CV) and semi-preparative HPLC, the title compound (Yield: 5%, 25 mg) as a beige foam. MS m/z (ES) [M+H]⁺: 593.42

¹H NMR (300 MHz, DMSO-d₆) δ 10.63 (s, 1H), 9.02 (s, 1H), 8.60 (d, J=5.6 Hz, 1H), 8.40 (s, 1H), 7.86 (d, J=8.7 Hz, 1H), 7.86 (d, J=8.8 Hz, 1H), 7.53-7.51 (m, 4H), 7.40-7.01 (m, 6H), 6.36 (s, 1H), 2.43 (s, 3H), 1.26 (s, 9H).

### Synthesis of Compound (cp): 1-(3-(tert-butyl)-1-phenyl-1H-pyrazol-5-yl)-3-(4-((3-oxo-3,4-dihydropyrido[2,3-b]pyrazin-8-yl)oxy)-3-(trifluoromethoxy)phenyl)urea

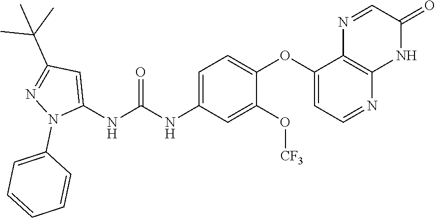 [SMILES: CC(C)(C)c1cc(NC(=O)Nc2ccc(Oc3ccnc4[nH]c(=O)cnc34)c(OC(F)(F)F)c2)n(-c2ccccc2)n1]

### Synthesis of Compound 1.4: 1-(3-(tert-butyl)-1-phenyl-1H-pyrazol-5-yl)-3-(4-((3-oxo-3,4-dihydropyrido[2,3-b]pyrazin-8-yl)oxy)-3-(trifluoromethoxy)phenyl)urea (Compound (cp))

According to Scheme 4 Step 2: In a sealed tube containing 8-[4-amino-2-(trifluoromethoxy)phenoxy]-4H-pyrido[2,3-b]pyrazin-3-one;hydrochloride 231 (150 mg, 0.46 mmol, 1.0 eq), DIEA (0.48 mL, 2.75 mmol, 6.0 eq) in DMSO (0.1M) was added 2,2,2,-trichloroethyl N-(5-tert-butyl-2-phenyl-pyrazol-3-yl)carbamate 158 (179 mg, 0.46 mmol, 1.0 eq). The reaction mixture was stirred at 80° C. for 20 h. The expected compound was obtained after 2 flash chromatographies (cyclohexane/ethyl acetate then ethyl acetate/methanol) and semi-preparative HPLC as beige solid (Yield: 33%, 76 mg). MS m/z (ES) [M+H]⁺: 580.50

### Synthesis of Compound (cq): 1-(3-(tert-butyl)-1-phenyl-1H-pyrazol-5-yl)-3-(2-fluoro-5-((3-oxo-3,4-dihydropyrido[2,3-b]pyrazin-8-yl)oxy)phenyl)urea

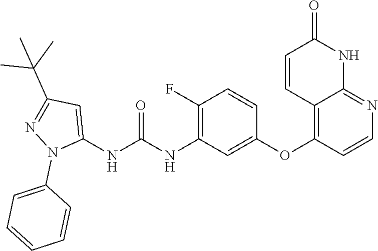 [SMILES: CC(C)(C)c1cc(NC(=O)Nc2cc(Oc3ccnc4[nH]c(=O)ccc34)ccc2F)n(-c2ccccc2)n1]

### Synthesis of Compound 1.4: 1-(3-(tert-butyl)-1-phenyl-1H-pyrazol-5-yl)-3-(2-fluoro-5-((3-oxo-3,4-dihydropyrido[2,3-b]pyrazin-8-yl)oxy)phenyl)urea (Compound (cq))

According to Scheme 4 Step 2: In a sealed tube containing 8-(3-amino-4-fluoro-phenoxy)-4H-pyrido[2,3-b]pyrazin-3-one hydrochloride 238 (300 mg, 0.97 mmol, 1.0 eq), DIEA (6.0 eq) in DMSO (0.1M) was added 2,2,2,-trichloroethyl N-(5-tert-butyl-2-phenyl-pyrazol-3-yl)carbamate 158 (759 mg, 1.94 mmol, 2.0 eq). The reaction mixture was stirred at 70° C. for 24 h. The precipitate was filtrated and the filtrate was purified by flash chromatography over silica gel using cyclohexane and ethyl acetate first and then ethyl acetate and methanol. The fractions were collected, evaporated and purified again by semi-preparative HPLC to afford, after basic extraction and lyophilization, the expected compound as white solid (Yield: 12%, 60 mg). MS m/z (ES) [M+H]⁺: 514.40

### Synthesis of Compound (cr): 1-(3-(tert-butyl)-1-(4-(3-(dimethylamino)propoxy)phenyl)-1H-pyrazol-5-yl)-3-(2-(methylthio)-4-((3-oxo-3,4-dihydropyrido[2,3-b]pyrazin-8-yl)oxy)phenyl)urea

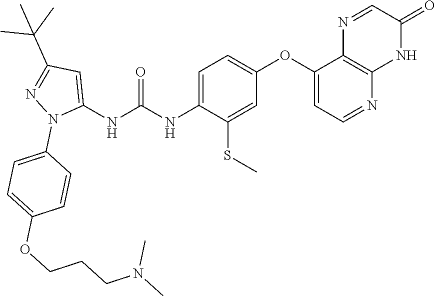 [SMILES: CSc1cc(Oc2ccnc3[nH]c(=O)cnc23)ccc1NC(=O)Nc1cc(C(C)(C)C)nn1-c1ccc(OCCCN(C)C)cc1]

### Synthesis of Compound 1.4: 1-(3-(tert-butyl)-1-(4-(3-(dimethylamino)propoxy)phenyl)-1H-pyrazol-5-yl)-3-(2-(methylthio)-4-((3-oxo-3,4-dihydropyrido[2,3-b]pyrazin-8-yl)oxy)phenyl)urea (Compound (cr))

According to Scheme 4 Step 2: In a sealed tube containing 8-(4-amino-3-(methylthio)phenoxy)pyrido[2,3-b]pyrazin-3(4H)-one, hydrochloride 111 (489 mg, 1.45 mmol, 1.0 eq), DIEA (6 eq) in DMSO (0.1M) was added 2,2,2-trichloroethyl N-[5-tert-butyl-2-[4-[3-(dimethylamino)propoxy]phenyl]pyrazol-3-yl]carbamate 240 (1.07 g, 2.17 mmol, 1.5 eq). The reaction mixture was stirred at 70° C. for 3 days. The reaction mixture was evaporated, and the residue was purified by flash chromatography over silica gel using cyclohexane and ethyl acetate and then ethyl acetate and methanol. The compound obtained was purified by flash chromatography over silica gel using first cyclohexane and ethyl acetate, then ethyl acetate and methanol, and then dichloromethane and ammoniac 7N in methanol. The residue obtained was purified again by semi-preparative HPLC to afford the expected compound as yellow solid (Yield: 4%, 45 mg). MS m/z (ES) [M+H]⁺: 643.42

### Synthesis of Compound (cs): 1-(3-(tert-butyl)-1-phenyl-1H-pyrazol-5-yl)-3-(4-isonicotinoylphenyl)urea

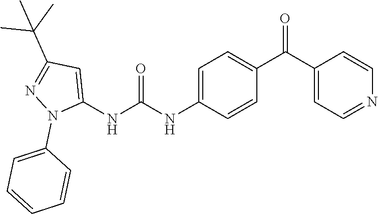 [SMILES: CC(C)(C)c1cc(NC(=O)Nc2ccc(C(=O)c3ccncc3)cc2)n(-c2ccccc2)n1]

### Synthesis of Compound 1.4: 1-(3-(tert-butyl)-1-phenyl-1H-pyrazol-5-yl)-3-(4-isonicotinoylphenyl)urea (Compound (cs))

According to Scheme 4 Step 2: In a sealed tube containing (4-aminophenyl)-(4-pyridyl)methanone (150 mg, 0.76 mmol, 1.0 eq) in acetonitrile (2 mL) was added 2,2,2-trichloroethyl N-(5-tert-butyl-2-phenyl-pyrazol-3-yl)carbamate 158 (591 mg, 1.52 mmol, 2.0 eq). The reaction mixture was stirred at 70° C. for 3 days. After cooling down, the mixture was directly absorbed on silica gel to be purified by flash chromatography using cyclohexane and ethyl acetate followed by a semi-preparative HPLC (proceeded on only 100 mg of compound). The expected compound was obtained as white foam after extraction with a saturated solution of sodium hydrogencarbonate and ethyl acetate followed by lyophilisation (Yield: 20%, 67 mg). MS m/z (ES) [M+H]⁺: 440.50

### Synthesis of Compound (ct): 1-(3-(tert-butyl)-1-phenyl-1H-pyrazol-5-yl)-3-(2-(methylthio)-4-((3-oxo-3,4-dihydro-2H-pyrido[3,2-b][1,4]oxazin-8-yl)oxy)phenyl)urea

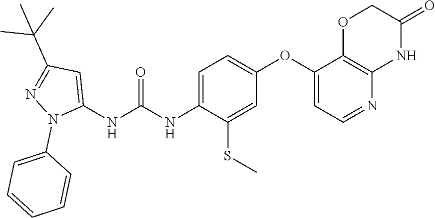 [SMILES: CSc1cc(Oc2ccnc3c2OCC(=O)N3)ccc1NC(=O)Nc1cc(C(C)(C)C)nn1-c1ccccc1]

### Synthesis of Compound 1.4: 1-(3-(tert-butyl)-1-phenyl-1H-pyrazol-5-yl)-3-(2-(methylthio)-4-((3-oxo-3,4-dihydro-2H-pyrido[3,2-b][1,4]oxazin-8-yl)oxy)phenyl)urea (Compound (ct))

According to Scheme 4 Step 2: In a sealed tube containing 8-(4-amino-3-methylsulfanyl-phenoxy)-4H-pyrido[3,2-b][1,4]oxazin-3-one hydrochloride 246 (116 mg, 0.38 mmol), DIEA (0.40 mL, 2.29 mmol) in DMSO (0.1M) was added 2,2,2-trichloroethyl N-(5-tert-butyl-2-phenyl-pyrazol-3-yl)carbamate 158 (224 mg, 0.57 mmol). The reaction mixture was stirred at 70° C. for 3 days. After filtration and semi-preparative HPLC, the title compound was obtained as a white powder (Yield: 30%, 63 mg). MS m/z (ES) [M+H]⁺: 545.50

¹H NMR (300 MHz, DMSO-d₆) δ 11.30 (s, 1H), 8.93 (s, 1H), 8.32 (s, 1H), 7.77 (d, J=5.6 Hz, 1H), 7.65 (d, J=8.8 Hz, 1H), 7.52-7.50 (m, 4H), 7.42-7.37 (m, 1H), 7.06 (d, J=2.7 Hz, 1H), 6.88 (dd, J=8.8, 2.7 Hz, 1H), 6.48 (d, J=5.6 Hz, 1H), 6.33 (s, 1H), 4.63 (s, 2H), 2.40 (s, 3H), 1.25 (s, 9H).

### Synthesis of Compound (cu): 1-(5-(tert-butyl)thiazol-2-yl)-3-(4-((7-oxo-5,6,7,8-tetrahydro-1,8-naphthyridin-4-yl)oxy)naphthalen-1-yl)urea

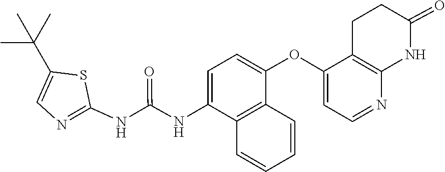 [SMILES: CC(C)(C)c1cnc(NC(=O)Nc2ccc(Oc3ccnc4c3CCC(=O)N4)c3ccccc23)s1]

### Synthesis of Compound 1.4: 1-(5-(tert-butyl)thiazol-2-yl)-3-(4-((7-oxo-5,6,7,8-tetrahydro-1,8-naphthyridin-4-yl)oxy)naphthalen-1-yl)urea (Compound (cu))

According to Scheme 4 Step 2: In a sealed tube containing 5-[(4-amino-1-naphthyl)oxy]-3,4-dihydro-1H-1,8-naphthyridin-2-one 247 (120 mg, 0.20 mmol), DIEA (0.30 mL, 1.90 mmol) in DMSO (0.1M) was added 2,2,2-trichloroethyl N-(5-tert-butylthiazol-2-yl)carbamate 157 (100 mg, 0.30 mmol). The reaction mixture was stirred at 70° C. for 18 h to furnish after flash chromatography (12 g, SiO₂, cyclohexane/AcOEt 100:0→0:100, 10 CV and AcOEt/CH₃OH 100:0→80:20, 5 CV) and semi-preparative HPLC, the title compound (Yield: 36%, 35 mg) as a purple solid. MS m/z (ES) [M+H]⁺: 488.33

¹H NMR (300 MHz, DMSO-d₆) δ 10.70 (br s, 1H), 10.52 (s, 1H), 9.27 (s, 1H), 8.14 (d, J=8.5 Hz, 1H), 7.92-7.86 (m, 3H), 7.69 (t, J=7.0 Hz, 1H), 7.59 (t, J=7.3 Hz, 1H), 7.27 (d, J=8.4 Hz, 1H), 7.08 (s, 1H), 6.10 (d, J=5.8 Hz, 1H), 3.07 (t, J=7.4 Hz, 2H), 2.62 (t, J=7.5 Hz, 2H), 1.31 (s, 9H).

### Synthesis of Compound (cv): Ethyl (4-(4-(3-(3-(tert-butyl)-1-phenyl-1H-pyrazol-5-yl)ureido)-3-(methylthio)phenoxy)pyridin-2-yl)carbamate

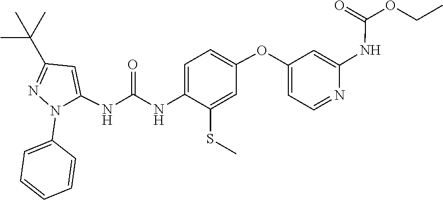 [SMILES: CCOC(=O)Nc1cc(Oc2ccc(NC(=O)Nc3cc(C(C)(C)C)nn3-c3ccccc3)c(SC)c2)ccn1]

### Synthesis of Compound 1.4: Ethyl (4-(4-(3-(3-(tert-butyl)-1-phenyl-1H-pyrazol-5-yl)ureido)-3-(methylthio)phenoxy)pyridin-2-yl)carbamate (Compound (cv))

According to Scheme 4 Step 2: In a sealed tube containing ethyl N-[4-(4-amino-3-methylsulfanyl-phenoxy)-2-pyridyl]carbamate hydrochloride 256 (76 mg, 0.24 mmol), DIEA (0.25 mL, 1.43 mmol) in DMSO (0.1M) was added 2,2,2-trichloroethyl N-(5-tert-butyl-2-phenyl-pyrazol-3-yl)carbamate 158 (140 mg, 0.36 mmol). The reaction mixture was stirred at 70° C. for 24 h to furnish after filtration and semi-preparative HPLC, the title compound (Yield: 8%, 11 mg) as a white solid. MS m/z (ES) [M+H]⁺: 561.42

¹H NMR (300 MHz, DMSO-d₆) δ 10.16 (s, 1H), 9.00 (s, 1H), 8.37 (s, 1H), 8.11 (d, J=5.7 Hz, 1H), 7.73 (d, J=8.8 Hz, 1H), 7.53-7.51 (m, 4H), 7.42-7.37 (m, 2H), 7.12 (d, J=2.7 Hz, 1H), 6.96 (dd, J=8.8, 2.7 Hz, 1H), 6.60 (dd, J=5.7, 2.3 Hz, 1H), 6.35 (s, 1H), 4.07 (qd, J=7.0 Hz, 2H), 2.41 (s, 3H), 1.26 (s, 9H), 1.17 (t, J=7.1 Hz, 3H).

### Synthesis of Compound (cw): 1-(3-(tert-butyl)-1-phenyl-1H-pyrazol-5-yl)-3-(2-fluoro-3-((3-oxo-3,4-dihydropyrido[2,3-b]pyrazin-8-yl)oxy)phenyl)urea

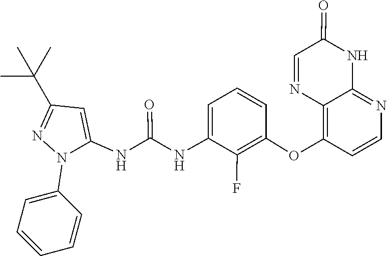 [SMILES: CC(C)(C)c1cc(NC(=O)Nc2cccc(Oc3ccnc4[nH]c(=O)cnc34)c2F)n(-c2ccccc2)n1]

### Synthesis of Compound 1.4: 1-(3-(tert-butyl)-1-phenyl-1H-pyrazol-5-yl)-3-(2-fluoro-3-((3-oxo-3,4-dihydropyrido[2,3-b]pyrazin-8-yl)oxy)phenyl)urea (Compound (cw))

According to Scheme 4 Step 2: In a sealed tube containing 8-(3-amino-2-fluoro-phenoxy)-4H-pyrido[2,3-b]pyrazin-3-one hydrochloride 263 (170 mg, 0.55 mmol, 1.0 eq), DIEA (6 eq.) in DMSO (0.1M) was added 2,2,2,-trichloroethyl N-(5-tert-butyl-2-phenyl-pyrazol-3-yl)carbamate 158 (324 mg, 0.83 mmol, 1.5 eq). The reaction mixture was stirred at 70° C. for 24 h. The expected compound was obtained after 2 flash chromatographies over silica gel using cyclohexane and ethyl acetate first and then ethyl acetate and methanol. The fractions were collected, evaporated and purified again in semi-preparative HPLC to afford, after lyophilization, the expected compound as orange foam (Yield: 9%, 37 mg). MS m/z (ES) [M+H]⁺: 514.42

### Synthesis of Compound (cx): Benzyl (4-(4-(3-(3-(tert-butyl)-1-phenyl-1H-pyrazol-5-yl)ureido)-3-(methylthio)phenoxy)pyridin-2-yl)carbamate

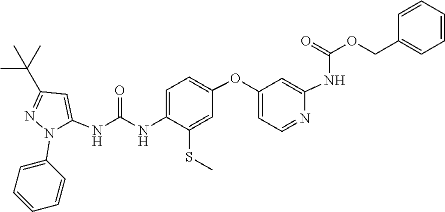 [SMILES: CSc1cc(Oc2ccnc(NC(=O)OCc3ccccc3)c2)ccc1NC(=O)Nc1cc(C(C)(C)C)nn1-c1ccccc1]

### Synthesis of Compound 1.4: Benzyl (4-(4-(3-(3-(tert-butyl)-1-phenyl-1H-pyrazol-5-yl)ureido)-3-(methylthio)phenoxy)pyridin-2-yl)carbamate (Compound (cx))

According to Scheme 4 Step 2: In a sealed tube containing benzyl N-[4-(4-amino-3-methylsulfanyl-phenoxy)-2-pyridyl]carbamate hydrochloride 266 (74 mg, 0.22 mmol), DIEA (0.23 mL, 1.35 mmol) in DMSO (0.1M) was added 2,2,2-trichloroethyl N-(5-tert-butyl-2-phenyl-pyrazol-3-yl)carbamate 158 (131 mg, 0.34 mmol). The reaction mixture was stirred at 70° C. for 24 h to furnish, after flash chromatography (12 g, SiO₂, cyclohexane/AcOEt 100:0→40:60, 10 CV) and semi-preparative HPLC, the title compound (Yield: 4%, 5 mg) as a beige solid. MS m/z (ES) [M+H]⁺: 561.42

¹H NMR (300 MHz, DMSO-d₆) δ 10.34 (s, 1H), 9.01 (s, 1H), 8.38 (s, 1H), 8.12 (d, J=5.7 Hz, 1H), 7.73 (d, J=8.8 Hz, 1H), 7.53-7.51 (m, 4H), 7.42-7.30 (m, 7H), 7.12 (d, J=2.6 Hz, 1H), 6.96 (dd, J=8.8, 2.6 Hz, 1H), 6.62 (dd, J=5.7, 2.2 Hz, 1H), 5.11 (s, 2H), 2.41 (s, 3H), 1.26 (s, 9H).

### Synthesis of Compound (cy): 1-(3-(tert-butyl)-1-phenyl-1H-pyrazol-5-yl)-3-(4-((3,4-dihydro-2H-pyrido[3,2-b][1,4]oxazin-8-yl)oxy)-2-(methylthio)phenyl)urea

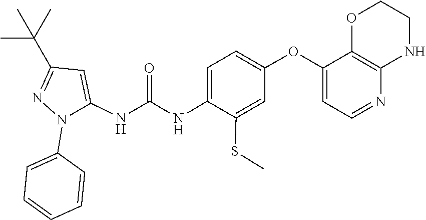 [SMILES: CSc1cc(Oc2ccnc3c2OCCN3)ccc1NC(=O)Nc1cc(C(C)(C)C)nn1-c1ccccc1]

### Synthesis of Compound 1.4: 1-(3-(tert-butyl)-1-phenyl-1H-pyrazol-5-yl)-3-(4-((3,4-dihydro-2H-pyrido[3,2-b][1,4]oxazin-8-yl)oxy)-2-(methylthio)phenyl)urea (Compound (cy))

According to Scheme 4 Step 2: In a sealed tube containing 4-(3,4-dihydro-2H-pyrido[3,2-b][1,4]oxazin-8-yloxy)-methylsulfanyl-aniline dihydrochloride 271 (90 mg, 0.33 mmol), DIEA (0.35 mL, 1.98 mmol) in DMSO (0.1M) was added 2,2,2-trichloroethyl N-(5-tert-butyl-2-phenyl-pyrazol-3-yl)carbamate 158 (190 mg, 0.50 mmol). The reaction mixture was stirred at 70° C. for 18 h to furnish, after flash chromatography (12 g, SiO₂, cyclohexane/AcOEt 100:0→0:100, 10 CV followed by AcOEt/CH₃OH 100:0→80:20, 5CV) and semi-preparative HPLC, the title compound (Yield: 12%, 21 mg) as a beige foam. MS m/z (ES) [M+H]⁺: 531.50

¹H NMR (300 MHz, DMSO-d₆) δ 8.88 (s, 1H), 8.27 (s, 1H), 7.56 (d, J=8.8 Hz, 1H), 7.52-7.50 (m, 4H), 7.46 (d, J=5.6 Hz, 1H), 7.41-7.37 (m, 1H), 6.95 (d, J=2.7 Hz, 1H), 6.80 (s, 1H), 6.75 (dd, J=8.8, 2.7 Hz, 1H), 6.33 (s, 1H), 6.06 (d, J=5.6 Hz, 1H), 4.07 (t, J=3.9 Hz, 2H), 3.38 (t, J=3.9 Hz, 2H), 2.38 (s, 3H), 1.25 (s, 9H).

### Synthesis of Compound (cz): 1-(3-(tert-butyl)isoxazol-5-yl)-3-(2-(methylthio)-4-((7-oxo-5,6,7,8-tetrahydro-1,8-naphthyridin-4-yl)oxy)phenyl)urea

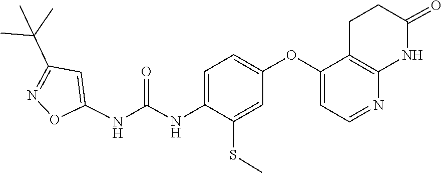 [SMILES: CSc1cc(Oc2ccnc3c2CCC(=O)N3)ccc1NC(=O)Nc1cc(C(C)(C)C)no1]

### Synthesis of Compound 1.4: 1-(3-(tert-butyl)isoxazol-5-yl)-3-(2-(methylthio)-4-((7-oxo-5,6,7,8-tetrahydro-1,8-naphthyridin-4-yl)oxy)phenyl)urea (Compound (cz))

According to Scheme 4 Step 2: In a sealed tube containing 5-(4-amino-3-methylsulfanyl-phenoxy)-3,4-dihydro-1H-1,8-naphthyridin-2-one 132 (40 mg, 0.13 mmol), DIEA (0.15 ml, 0.80 mmol) in DMSO (0.1M) was added 2,2,2-trichloroethyl N-(3-tert-butylisoxazol-5-yl)carbamate 273 (82 mg, 0.20 mmol). The reaction mixture was stirred at 70° C. for 18 h to furnish, after filtration and semi-preparative HPLC, the title compound (Yield: 29%, 18 mg) as a beige foam. MS m/z (ES) [M+H]⁺: 468.33

¹H NMR (300 MHz, DMSO-d₆) δ 10.68 (br s, 1H), 10.51 (s, 1H), 8.27 (s, 1H), 7.96 (d, J=5.8 Hz, 1H), 7.79 (d, J=8.8 Hz, 1H), 7.15 (d, J=2.4 Hz, 1H), 6.95 (dd, J=8.8, 2.5 Hz, 1H), 6.31 (s, 1H), 6.01 (s, 1H), 2.90 (t, J=7.7 Hz, 2H), 2.51 (t, J=7.7 Hz, 2H), 2.43 (s, 3H), 1.23 (s, 9H).

### Synthesis of Compound (da): Ethyl (4-((4-(3-(3-(tert-butyl)-1-phenyl-1H-pyrazol-5-yl)ureido)naphthalen-1-yl)oxy)pyridin-2-yl)carbamate

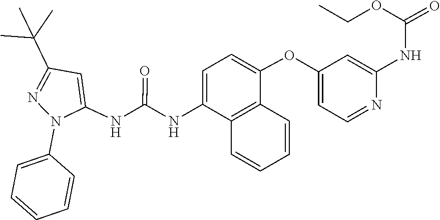 [SMILES: CCOC(=O)Nc1cc(Oc2ccc(NC(=O)Nc3cc(C(C)(C)C)nn3-c3ccccc3)c3ccccc23)ccn1]

### Synthesis of Compound 1.4: Ethyl (4-((4-(3-(3-(tert-butyl)-1-phenyl-1H-pyrazol-5-yl)ureido)naphthalen-1-yl)oxy)pyridin-2-yl)carbamate (Compound (da))

According to Scheme 4 Step 2: In a sealed tube containing ethyl N-{4-[(4-amino-1-naphthyl)oxy]-2-pyridyl}carbamate 252 (78 mg, 0.24 mmol), DIEA (0.21 mL, 1.21 mmol) in DMSO (0.1M) was added 2,2,2-trichloroethyl N-(5-tert-butyl-2-phenyl-pyrazol-3-yl)carbamate 158 (141 mg, 0.36 mmol). The reaction mixture was stirred at 70° C. for 5 h to furnish, after filtration and semi-preparative HPLC, the title compound (Yield: 24%, 33 mg) as a white solid. 565.42

¹H NMR (300 MHz, DMSO-d₆) δ 10.14 (s, 1H), 9.12 (s, 1H), 8.83 (s, 1H), 8.10 (d, J=5.7 Hz, 1H), 8.05 (d, J=8.4 Hz, 1H), 7.93 (d, J=8.4 Hz, 1H), 7.81 (d, J=7.6 Hz, 1H), 7.65-7.52 (m, 6H), 7.45-7.40 (m, 1H), 7.35-7.30 (m, 2H), 6.62 (dd, J=5.7, 2.3 Hz, 1H), 6.41 (s, 1H), 4.01 (qd, J=7.1 Hz, 2H), 1.27 (s, 9H), 1.13 (t, J=7.1 Hz, 3H).

### Synthesis of Compound (db): Ethyl (4-((4-(3-(3-(tert-butyl)isoxazol-5-yl)ureido)naphthalen-1-yl)oxy)pyridin-2-yl)carbamate

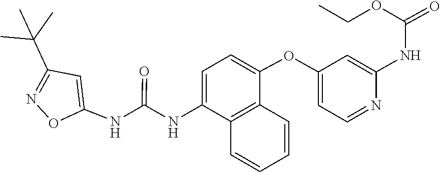 [SMILES: CCOC(=O)Nc1cc(Oc2ccc(NC(=O)Nc3cc(C(C)(C)C)no3)c3ccccc23)ccn1]

### Synthesis of Compound 1.4: Ethyl (4-((4-(3-(3-(tert-butyl)isoxazol-5-yl)ureido)naphthalen-1-yl)oxy)pyridin-2-yl)carbamate (Compound (db))

According to Scheme 4 Step 2: In a sealed tube containing ethyl N-{4-[(4-amino-1-naphthyl)oxy]-2-pyridyl}carbamate 252 (70 mg, 0.22 mmol), DIEA (0.19 mL, 1.08 mmol) in DMSO (0.1M) was added 2,2,2-trichloroethyl N-(3-tert-butylisoxazol-5-yl)carbamate 273 (102 mg, 0.32 mmol). The reaction mixture was stirred at 70° C. for 6 h to furnish, after filtration and semi-preparative HPLC, the title compound (Yield: 28%, 30 mg) as a white solid. MS m/z (ES) [M+H]⁺: 490.42

¹H NMR (300 MHz, DMSO-d₆) δ 10.41 (s, 1H), 10.15 (s, 1H), 9.00 (s, 1H), 8.11 (d, J=5.9 Hz, 2H), 7.97 (d, J=8.3 Hz, 1H), 7.85 (d, J=7.7 Hz, 1H), 7.69 (t, J=7.0 Hz, 1H), 7.58 (t, J=7.2 Hz, 1H), 7.37 (s, 1H), 7.34 (d, J=8.6 Hz, 1H), 6.64 (dd, J=5.7, 2.3 Hz, 1H), 6.07 (s, 1H), 4.01 (qd, J=7.1 Hz, 2H), 1.24 (s, 9H), 1.13 (t, J=7.1 Hz, 3H).

### Example: Synthesis of Compound (dc): Ethyl (4-((4-(3-(5-(tert-butyl)thiazol-2-yl)ureido)naphthalen-1-yl)oxy)pyridin-2-yl)carbamate

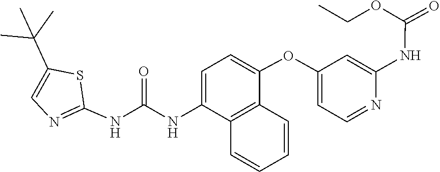 [SMILES: CCOC(=O)Nc1cc(Oc2ccc(NC(=O)Nc3ncc(C(C)(C)C)s3)c3ccccc23)ccn1]

### Synthesis of Compound 1.4: Ethyl (4-((4-(3-(5-(tert-butyl)thiazol-2-yl)ureido)naphthalen-1-yl)oxy)pyridin-2-yl)carbamate (Compound (dc))

According to Scheme 4 Step 2: In a sealed tube containing Ethyl N-{4-[(4-amino-1-naphthyl)oxy]-2-pyridyl}carbamate 252 (70 mg, 0.22 mmol), DIEA (0.19 ml, 1.08 mmol) in DMSO (0.1M) was added 2,2,2-trichloroethyl N-(5-tert-butylthiazol-2-yl)carbamate 157 (108 mg, 0.32 mmol). The reaction mixture was stirred at 70° C. for 6 h to furnish, after filtration and semi-preparative HPLC, the title compound (Yield: 16%, 18 mg) as a white solid. MS m/z (ES) [M+H]⁺: 506.25

¹H NMR (300 MHz, DMSO-d₆) δ 10.68 (s, 1H), 10.15 (s, 1H), 9.27 (br s, 1H), 8.16-8.10 (m, 2H), 8.05 (d, J=8.3 Hz, 1H), 7.85 (d, J=8.0 Hz, 1H), 7.69 (t, J=6.9 Hz, 1H), 7.59 (t, J=7.3 Hz, 1H), 7.36 (s, 1H), 7.34 (d, J=6.8 Hz, 1H), 7.09 (s, 1H), 6.64 (dd, J=5.7, 2.3 Hz, 1H), 4.01 (qd, J=7.1 Hz, 2H), 1.31 (s, 9H), 1.13 (t, J=7.1 Hz, 3H).

### Synthesis of Compound (dd): 1-(3-(tert-butyl)-1-(4-(morpholinomethyl)phenyl)-1H-pyrazol-5-yl)-3-(2-(methylthio)-4-((3-oxo-3,4-dihydropyrido[2,3-b]pyrazin-8-yl)oxy)phenyl)urea

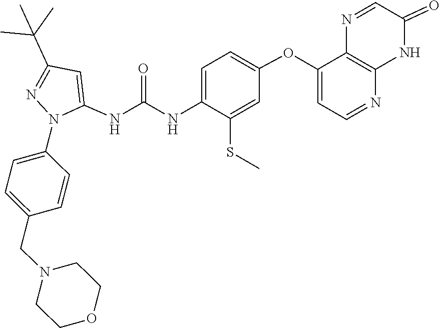 [SMILES: CSc1cc(Oc2ccnc3[nH]c(=O)cnc23)ccc1NC(=O)Nc1cc(C(C)(C)C)nn1-c1ccc(CN2CCOCC2)cc1]

### Synthesis of Compound 1.4: 1-(3-(tert-butyl)-1-(4-(morpholinomethyl)phenyl)-1H-pyrazol-5-yl)-3-(2-(methylthio)-4-((3-oxo-3,4-dihydropyrido[2,3-b]pyrazin-8-yl)oxy)phenyl)urea (Compound (dd))

According to Scheme 4 Step 2: In a sealed tube containing 8-(4-amino-3-(methylthio)phenoxy)pyrido[2,3-b]pyrazin-3(4H)-one, hydrochloride 111 (1.33 g, 3.95 mmol, 1.0 eq), DIEA (6 eq) in DMSO (0.1M) was added 2,2,2-trichloroethyl N-[5-tert-butyl-2-[4-(morpholinomethyl)phenyl]pyrazol-3-yl]carbamate 279 (2.13 g, 4.35 mmol, 1.1 eq). The reaction mixture was stirred at 70° C. for 24 h. The precipitate and filtrate were independently triturated in dichloromethane and ethyl acetate to try to precipitate. Finally, the different filtrates were combined and purified by flash chromatography over silica gel using dichloromethane and ethyl acetate first, and then ethyl acetate and methanol. The fractions were combined and purified again by flash chromatography over silica gel to afford expected compound as orange powder (Yield: 11%, 280 mg). MS m/z (ES) [M+H]⁺: 641.50

### Synthesis of Compound (de): Ethyl (4-(4-(3-(3-(tert-butyl)isoxazol-5-yl)ureido)-3-(methylthio)phenoxy)pyridin-2-yl)carbamate

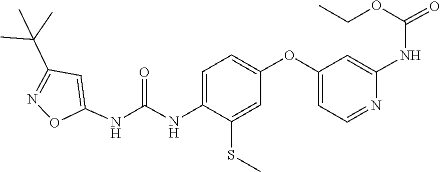 [SMILES: CCOC(=O)Nc1cc(Oc2ccc(NC(=O)Nc3cc(C(C)(C)C)no3)c(SC)c2)ccn1]

### Synthesis of Compound 1.4: Ethyl (4-(4-(3-(3-(tert-butyl)isoxazol-5-yl)ureido)-3-(methylthio)phenoxy)pyridin-2-yl)carbamate (Compound (de))

According to Scheme 4 Step 2: In a sealed tube containing Ethyl N-[4-(4-amino-3-methylsulfanyl-phenoxy)-2-pyridyl]carbamate hydrochloride 256 (80 mg, 0.25 mmol), DIEA (0.22 mL, 1.25 mmol) in DMSO (0.1M) was added 2,2,2-trichloroethyl N-(3-tert-butylisoxazol-5-yl)carbamate 273 (119 mg, 0.38 mmol). The reaction mixture was stirred at 70° C. for 24 h to furnish, after filtration and semi-preparative HPLC, the title compound (Yield: 27%, 33 mg) as a white solid. MS m/z (ES) [M+H]⁺: 486.33

¹H NMR (300 MHz, DMSO-d₆) δ 10.69 (s, 1H), 10.18 (s, 1H), 8.28 (s, 1H), 8.12 (d, J=5.7 Hz, 1H), 7.82 (d, J=8.8 Hz, 1H), 7.38 (d, J=2.2 Hz, 1H), 7.18 (d, J=2.7 Hz, 1H), 7.02 (dd, J=8.8, 2.7 Hz, 1H), 6.62 (dd, J=5.7, 2.3 Hz, 1H), 6.02 (s, 1H), 4.06 (qd, J=7.1 Hz, 2H), 2.44 (s, 3H), 1.23 (s, 9H), 1.17 (t, J=7.1 Hz, 3H).

### Synthesis of Compound (df): Ethyl (4-(4-(3-(5-(tert-butyl)thiazol-2-yl)ureido)-3-(methylthio)phenoxy)pyridin-2-yl)carbamate

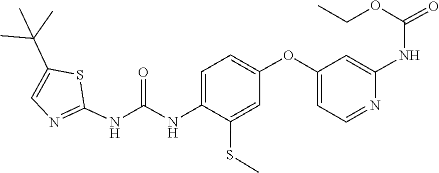 [SMILES: CCOC(=O)Nc1cc(Oc2ccc(NC(=O)Nc3ncc(C(C)(C)C)s3)c(SC)c2)ccn1]

### Synthesis of Compound 1.4: Ethyl (4-(4-(3-(5-(tert-butyl)thiazol-2-yl)ureido)-3-(methylthio)phenoxy)pyridin-2-yl)carbamate (Compound (df))

According to Scheme 4 Step 2: In a sealed tube containing ethyl N-[4-(4-amino-3-methylsulfanyl-phenoxy)-2-pyridyl]carbamate hydrochloride 256 (80 mg, 0.25 mmol), DIEA (0.22 mL, 1.25 mmol) in DMSO (0.1M) was added 2,2,2-trichloroethyl N-(5-tert-butylthiazol-2-yl)carbamate 157 (125 mg, 0.38 mmol). The reaction mixture was stirred at 70° C. for 24 h to furnish, after flash chromatography (12 g, SiO₂, cyclohexane/AcOEt 100:0→40:60, 10 CV) and semi-preparative HPLC, the title compound (Yield: 12%, 15 mg) as a white foam. MS m/z (ES) [M+H]⁺: 502.33

¹H NMR (300 MHz, DMSO-d₆) δ 10.93 (s, 1H), 10.17 (s, 1H), 8.60 (br s, 1H), 8.11 (d, J=5.7 Hz, 1H), 7.86 (d, J=8.8 Hz, 1H), 7.39 (d, J=2.3 Hz, 1H), 7.18 (d, J=2.7 Hz, 1H), 7.05 (s, 1H), 7.01 (dd, J=8.8, 2.7 Hz, 1H), 6.62 (dd, J=5.7, 2.3 Hz, 1H), 4.07 (qd, J=7.1 Hz, 2H), 2.44 (s, 3H), 1.30 (s, 9H), 1.17 (t, J=7.1 Hz, 3H).

### Synthesis of Compound (dg): 1-(3-(tert-butyl)isoxazol-5-yl)-3-(4-((7-oxo-5,6,7,8-tetrahydro-1,8-naphthyridin-4-yl)oxy)naphthalen-1-yl)urea

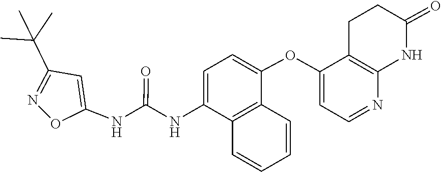 [SMILES: CC(C)(C)c1cc(NC(=O)Nc2ccc(Oc3ccnc4c3CCC(=O)N4)c3ccccc23)on1]

### Synthesis of Compound 1.4: 1-(3-(tert-butyl)isoxazol-5-yl)-3-(4-((7-oxo-5,6,7,8-tetrahydro-1,8-naphthyridin-4-yl)oxy)naphthalen-1-yl)urea (Compound (dg))

According to Scheme 4 Step 2: In a sealed tube containing 5-[(4-amino-1-naphthyl)oxy]-3,4-dihydro-1H-1,8-naphthyridin-2-one 247 (80 mg, 0.25 mmol), DIEA (0.30 mL, 1.57 mmol) in DMSO (0.1M) was added 2,2,2-trichloroethyl N-(5-tert-butylthiazol-2-yl)carbamate 273 (125 mg, 0.38 mmol). The reaction mixture was stirred at 70° C. for 18 h to furnish, after flash chromatography (12 g, SiO₂, cyclohexane/AcOEt 100:0→0:100, 10 CV and AcOEt/CH₃OH 100:0→80:20, 5 CV) and semi-preparative HPLC, the title compound (Yield: 10%, 12 mg) as a purple powder. MS m/z (ES) [M+H]⁺: 472.33

¹H NMR (300 MHz, DMSO-d₆) δ 10.54 (s, 1H), 10.45 (br s, 1H), 9.02 (s, 1H), 8.11 (d, J=8.5 Hz, 1H), 7.94-7.87 (m, 3H), 7.69 (t, J=7.2 Hz, 1H), 7.59 (t, J=7.6 Hz, 1H), 7.27 (d, J=8.3 Hz, 1H), 6.11 (d, J=5.7 Hz, 1H), 6.05 (s, 1H), 3.07 (t, J=7.5 Hz, 2H), 2.62 (t, J=7.5 Hz, 2H), 1.24 (s, 9H).

### Synthesis of Compound (dh): 1-(5-(tert-butyl)isoxazol-3-yl)-3-(2-(methylthio)-4-((3-oxo-3,4-dihydropyrido[2,3-b]pyrazin-8-yl)oxy)phenyl)urea

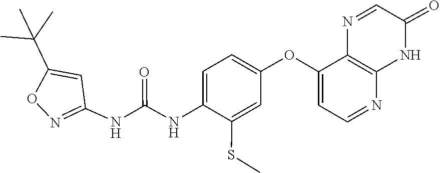 [SMILES: CSc1cc(Oc2ccnc3[nH]c(=O)cnc23)ccc1NC(=O)Nc1cc(C(C)(C)C)on1]

### Synthesis of Compound 1.4: 1-(5-(tert-butyl)isoxazol-3-yl)-3-(2-(methylthio)-4-((3-oxo-3,4-dihydropyrido[2,3-b]pyrazin-8-yl)oxy)phenyl)urea (Compound (dh))

According to Scheme 4 Step 2: In a sealed tube containing 8-(4-amino-3-(methylthio)phenoxy)pyrido[2,3-b]pyrazin-3(4H)-one, hydrochloride 111 (150 mg, 0.44 mmol, 1.0 eq), DIEA (6 eq) in DMSO (0.1M) was added 2,2,2-trichloroethyl N-(5-tert-butylisoxazol-3-yl)carbamate 275 (211 mg, 0.67 mmol, 1.5 eq). The reaction mixture was stirred at 70° C. for 24 h. The precipitate was filtered, and the filtrate was purified by flash chromatography over silica gel first using cyclohexane and ethyl acetate and then using ethyl acetate and methanol. The compound obtained was purified again by semi-preparative HPLC to afford expected compound as salmon powder (Yield: 2%, 4 mg). MS m/z (ES) [M+H]⁺: 467.33

### Synthesis of Compound (di): Ethyl (4-(4-(3-(5-(tert-butyl)isoxazol-3-yl)ureido)-3-(methylthio)phenoxy)pyridin-2-yl)carbamate

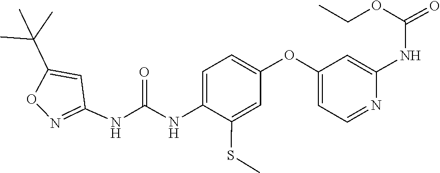 [SMILES: CCOC(=O)Nc1cc(Oc2ccc(NC(=O)Nc3cc(C(C)(C)C)on3)c(SC)c2)ccn1]

### Synthesis of Compound 1.4: Ethyl (4-(4-(3-(5-(tert-butyl)isoxazol-3-yl)ureido)-3-(methylthio)phenoxy)pyridin-2-yl)carbamate (Compound (di))

According to Scheme 4 Step 2: In a sealed tube containing ethyl N-[4-(4-amino-3-methylsulfanyl-phenoxy)-2-pyridyl]carbamate hydrochloride 256 (80 mg, 0.25 mmol), DIEA (0.22 mL, 1.25 mmol) in DMSO (0.1M) was added 2,2,2-trichloroethyl N-(5-tert-butylisoxazol-3-yl)carbamate 275 (119 mg, 0.38 mmol). The reaction mixture was stirred at 70° C. for 24 h to furnish, after flash chromatography (12 g, SiO₂, cyclohexane/AcOEt 100:0→20:80, 10 CV) and semi-preparative HPLC, the title compound (Yield: 6%, 8 mg) as a white solid. MS m/z (ES) [M+H]⁺: 486.33

¹H NMR (300 MHz, DMSO-d₆) δ 10.16 (s, 1H), 10.11 (s, 1H), 8.43 (s, 1H), 8.12 (d, J=5.7 Hz, 1H), 7.84 (d, J=8.8 Hz, 1H), 7.39 (d, J=2.1 Hz, 1H), 7.16 (d, J=2.5 Hz, 1H), 6.99 (dd, J=8.8, 2.4 Hz, 1H), 6.62 (dd, J=5.6, 2.2 Hz, 1H), 6.42 (s, 1H), 4.06 (qd, J=7.1 Hz, 2H), 2.44 (s, 3H), 1.27 (s, 9H), 1.17 (t, J=7.1 Hz, 3H).

### Synthesis of Compound (dj): N-(4-((4-(3-(3-(tert-butyl)-1-phenyl-1H-pyrazol-5-yl)ureido)naphthalen-1-yl)oxy)pyridin-2-yl)acetamide

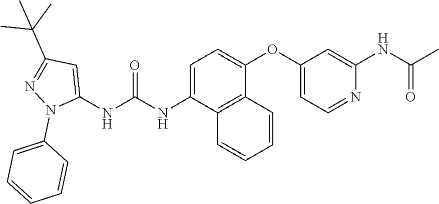 [SMILES: CC(=O)Nc1cc(Oc2ccc(NC(=O)Nc3cc(C(C)(C)C)nn3-c3ccccc3)c3ccccc23)ccn1]

### Synthesis of Compound 1.4: N-(4-((4-(3-(3-(tert-butyl)-1-phenyl-1H-pyrazol-5-yl)ureido)naphthalen-1-yl)oxy)pyridin-2-yl)acetamide (Compound (dj))

According to Scheme 4 Step 2: In a sealed tube containing N-{4-[(4-amino-1-naphthyl)oxy]-2-pyridyl}acetamide 281 (115 mg, 0.39 mmol), DIEA (0.70 mL, 3.90 mmol) in DMSO (0.1M) was added 2,2,2-trichloroethyl N-(5-tert-butyl-2-phenyl-pyrazol-3-yl) carbamate 158 (306 mg, 0.78 mmol). The reaction mixture was stirred at 70° C. for 24 h to furnish, after flash chromatography (12 g, SiO₂, cyclohexane/AcOEt 100:0→0:100, 10 CV and AcOEt/CH₃OH 100:0→80:20, 5 CV) and semi-preparative HPLC, the title compound (Yield: 12%, 25 mg) as a white solid. MS m/z (ES) [M+H]⁺: 535.42

¹H NMR (300 MHz, DMSO-d₆) δ 10.53 (s, 1H), 9.14 (s, 1H), 8.79 (s, 1H), 8.15 (d, J=5.7 Hz, 1H), 8.07 (d, J=8.5 Hz, 1H), 7.93 (d, J=8.4 Hz, 1H), 7.65 (d, J=2.4 Hz, 1H), 7.63-7.52 (m, 7H), 7.46-7.40 (m, 1H), 7.31 (d, J=8.3 Hz, 1H), 6.63 (dd, J=5.7, 2.4 Hz, 1H), 6.42 (s, 1H), 1.99 (s, 3H), 1.28 (s, 9H).

### Synthesis of Compound (dk): 1-(3-(tert-butyl)-1-phenyl-1H-pyrazol-5-yl)-3-(2-(methylthio)-4-(pyridin-4-yloxy)phenyl)urea

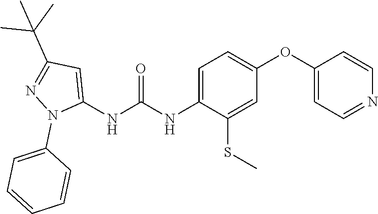 [SMILES: CSc1cc(Oc2ccncc2)ccc1NC(=O)Nc1cc(C(C)(C)C)nn1-c1ccccc1]

### Synthesis of Compound 1.4: 1-(3-(tert-butyl)-1-phenyl-1H-pyrazol-5-yl)-3-(2-(methylthio)-4-(pyridin-4-yloxy)phenyl)urea (Compound (dk))

According to Scheme 4 Step 2: In a sealed tube containing 2-methylsulfanyl-4-(4-pyridyloxy)aniline 283 (150 mg, 0.65 mmol), DIEA (6 eq.) in DMSO (0.1M) was added 2,2,2-trichloroethyl N-(5-tert-butyl-2-phenyl-pyrazol-3-yl)carbamate 158 (504 mg, 1.30 mmol). The reaction mixture was stirred at 70° C. for 24 h to furnish, after flash chromatography (12 g, SiO₂, cyclohexane/AcOEt 100:0→0:100, 10 CV then AcOEt/CH₃OH 100:0→80:20, 10 CV) and semi-preparative HPLC, the title compound (Yield: 14%, 44 mg) as a white solid. MS m/z (ES) [M+H]⁺: 474.33

¹H NMR (300 MHz, DMSO-d₆) δ 9.10 (s, 1H), 8.48 (s, 1H), 7.98 (d, J=7.8 Hz, 1H), 7.85 (d, J=8.8 Hz, 2H), 7.53-7.50 (m, 4H), 7.43-7.38 (m, 2H), 7.31 (dd, J=8.8, 2.6 Hz, 1H), 6.37 (s, 1H), 6.21 (d, J=7.7 Hz, 2H), 2.49 (s, 3H), 1.27 (s, 9H).

### Synthesis of Compound (dl): N-(4-(4-(3-(3-(tert-butyl)isoxazol-5-yl)ureido)-3-(methylthio)phenoxy)pyridin-2-yl)methanesulfonamide

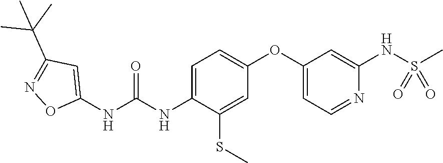 [SMILES: CSc1cc(Oc2ccnc(NS(C)(=O)=O)c2)ccc1NC(=O)Nc1cc(C(C)(C)C)no1]

### Synthesis of Compound 1.4: N-(4-(4-(3-(3-(tert-butyl)isoxazol-5-yl)ureido)-3-(methylthio)phenoxy)pyridin-2-yl)methanesulfonamide (Compound (dl))

According to Scheme 4 Step 2: In a sealed tube containing N-[4-(4-amino-3-methylsulfanyl-phenoxy)-2-pyridyl]-methanesulfonamide 285 (59 mg, 0.18 mmol), DIEA (0.20 mL, 0.73 mmol) in DMSO (0.1M) was added 2,2,2-trichloroethyl (3-(tert-butyl)isoxazol-5-yl)carbamate 273 (410 mg, 1.30 mmol). The reaction mixture was stirred at 70° C. for 36 h to furnish after semi-preparative HPLC the title compound (Yield: 5%, 4.8 mg) as a beige solid. MS m/z (ES) [M+H]⁺: 492.42

¹H NMR (300 MHz, DMSO-d₆) δ 10.87 (s, 1H), 8.45 (s, 1H), 8.02 (d, J=6.3 Hz, 1H), 7.81 (d, J=8.8 Hz, 1H), 7.19 (d, J=2.6 Hz, 1H), 7.02 (dd, J=8.8, 2.6 Hz, 1H), 6.54 (dd, J=8.4, 2.1 Hz, 1H), 6.46 (d, J=2.2 Hz, 1H), 6.01 (s, 1H), 3.10 (s, 3H), 2.44 (s, 3H), 1.23 (s, 9H).

### Synthesis of Compound (dm): 1-(3-(tert-butyl)-1-phenyl-1H-pyrazol-5-yl)-3-(2-(methylthio)-4-((2-oxo-2,3-dihydro-1H-imidazo[4,5-b]pyridin-7-yl)oxy)phenyl)urea

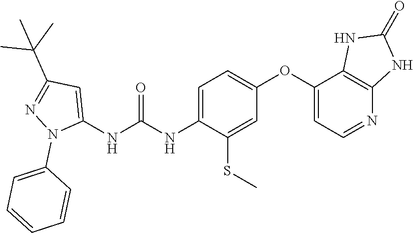 [SMILES: CSc1cc(Oc2ccnc3[nH]c(=O)[nH]c23)ccc1NC(=O)Nc1cc(C(C)(C)C)nn1-c1ccccc1]

### Synthesis of Compound 1.4: 1-(3-(tert-butyl)-1-phenyl-1H-pyrazol-5-yl)-3-(2-(methylthio)-4-((2-oxo-2,3-dihydro-1H-imidazo[4,5-b]pyridin-7-yl)oxy)phenyl)urea (Compound (dm))

According to Scheme 4 Step 2: In a sealed tube containing 7-(4-amino-3-methylsulfanyl-phenoxy)-1,3-dihydroimidazo[4,5-b]pyridin-2-one hydrochloride 287 (150 mg, 0.52 mmol), DIEA (0.90 mL, 5.2 mmol) in DMSO (0.1M) was added 2,2,2-trichloroethyl N-(5-tert-butyl-2-phenyl-pyrazol-3-yl)carbamate 158 (407 mg, 1.04 mmol). The reaction mixture was stirred at 70° C. for 24 h to furnish, after filtration and semi-preparative HPLC, the title compound (Yield: 6%, 17 mg) as a brown solid. MS m/z (ES) [M+H]⁺: 530.33

¹H NMR (300 MHz, DMSO-d₆) δ 11.38 (s, 1H), 11.20 (s, 1H), 8.99 (s, 1H), 8.37 (s, 1H), 7.75 (d, J=5.9 Hz, 1H), 7.68 (d, J=8.8 Hz, 1H), 7.53-7.51 (m, 4H), 7.44-7.37 (m, 1H), 7.12 (d, J=2.7 Hz, 1H), 6.94 (dd, J=8.8, 2.7 Hz, 1H), 6.38 (d, J=5.9 Hz, 1H), 6.34 (s, 1H), 2.41 (s, 3H), 1.26 (s, 9H).

### Synthesis of Compound (dn): 1-(3-(tert-butyl)-1-phenyl-1H-pyrazol-5-yl)-3-(3-(methylthio)-4-((3-oxo-3,4-dihydropyrido[2,3-b]pyrazin-8-yl)oxy)phenyl)urea

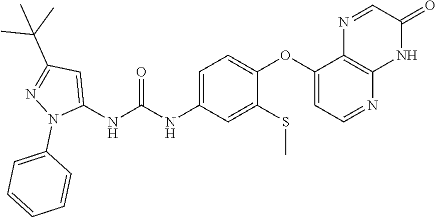 [SMILES: CSc1cc(NC(=O)Nc2cc(C(C)(C)C)nn2-c2ccccc2)ccc1Oc1ccnc2[nH]c(=O)cnc12]

### Synthesis of Compound 1.4: 1-(3-(tert-butyl)-1-phenyl-1H-pyrazol-5-yl)-3-(3-(methylthio)-4-((3-oxo-3,4-dihydropyrido[2,3-b]pyrazin-8-yl)oxy)phenyl)urea (Compound (dn))

According to Scheme 4 Step 2: In a sealed tube containing 8-(4-amino-2-methylsulfanyl-phenoxy]-4H-pyrido[2,3-b]pyrazin-3-one hydrochloride 296 (160 mg, 0.53 mmol), DIEA (0.91 mL, 5.3 mmol) in DMSO (0.1M) was added 2,2,2-trichloroethyl N-(5-tert-butyl-2-phenyl-pyrazol-3-yl)carbamate 158 (313 mg, 0.80 mmol). The reaction mixture was stirred at 70° C. for 20 h to furnish, after flash chromatography (40 g, SiO₂, cyclohexane/AcOEt 100:0→0:100, 10 CV) the title compound (Yield: 30%, 86 mg) as an orange solid. MS m/z (ES) [M+H]⁺: 542.42

¹H NMR (300 MHz, DMSO-d₆) δ 12.89 (s, 1H), 9.25 (s, 1H), 8.47 (s, 1H), 8.27 (d, J=5.7 Hz, 1H), 8.18 (s, 1H), 7.53-7.49 (m, 5H), 7.43-7.38 (m, 1H), 7.26 (dd, J=8.7, 2.3 Hz, 1H), 7.11 (d, J=8.7 Hz, 1H), 6.37 (s, 1H), 6.34 (d, J=5.7 Hz, 1H), 2.36 (s, 3H), 1.27 (s, 9H).

### Synthesis of Compound (do): 1-(3-(tert-butyl)-1-phenyl-1H-pyrazol-5-yl)-3-(2-(methylthio)-4-((7-oxo-7,8-dihydro-1,8-naphthyridin-4-yl)oxy)phenyl)urea

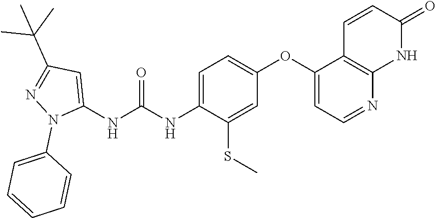 [SMILES: CSc1cc(Oc2ccnc3[nH]c(=O)ccc23)ccc1NC(=O)Nc1cc(C(C)(C)C)nn1-c1ccccc1]

### Synthesis of Compound 1.4: 1-(3-(tert-butyl)-1-phenyl-1H-pyrazol-5-yl)-3-(2-(methylthio)-4-((7-oxo-7,8-dihydro-1,8-naphthyridin-4-yl)oxy)phenyl)urea (Compound (do))

According to Scheme 4 Step 2: In a sealed tube containing 5-(4-amino-3-methylsulfanyl-phenoxy)-1H-1,8-naphthyridin-2-one 298 (244 mg, 0.81 mmol, 1.0 eq), DIEA (468 μL, 3.26 mmol, 4.0 eq) in DMSO (0.1M) was added 2,2,2,-trichloroethyl N-(5-tert-butyl-2-phenyl-pyrazol-3-yl)carbamate 158 (318 mg, 0.81 mmol, 1.0 eq). The reaction mixture was stirred at 80° C. for 18 h. After cooling down, the precipitate was filtered and purified by semi-preparative HPLC to afford after lyophilization the expected compound as grey solid (Yield: 8%, 34 mg). MS m/z (ES) [M+H]⁺: 541.30

### Synthesis of Compound (dp): 1-(3-(tert-butyl)-1-(4-((dimethylamino)methyl)phenyl)-1H-pyrazol-5-yl)-3-(2-(methylthio)-4-((3-oxo-3,4-dihydropyrido[2,3-b]pyrazin-8-yl)oxy)phenyl)urea

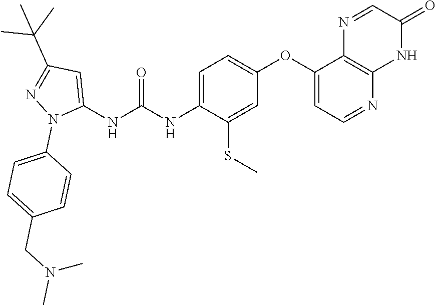 [SMILES: CSc1cc(Oc2ccnc3[nH]c(=O)cnc23)ccc1NC(=O)Nc1cc(C(C)(C)C)nn1-c1ccc(CN(C)C)cc1]

### Synthesis of Compound 1.4: 1-(3-(tert-butyl)-1-(4-((dimethylamino)methyl)phenyl)-1H-pyrazol-5-yl)-3-(2-(methylthio)-4-((3-oxo-3,4-dihydropyrido[2,3-b]pyrazin-8-yl)oxy)phenyl)urea (Compound (dp))

According to Scheme 4 Step 2: In a sealed tube containing 8-(4-amino-3-(methylthio)phenoxy)pyrido[2,3-b]pyrazin-3(4H)-one, hydrochloride 111 (266 mg, 0.79 mmol, 1.0 eq), DIEA (6 eq) in DMSO (0.1M) was added 2,2,2-trichloroethyl N-[5-tert-butyl-2-[4-[(dimethylamino)methyl]phenyl]pyrazol-3-yl]carbamate 303 (708 mg estimated, 1.58 mmol, 2.0 eq). The reaction mixture was stirred at 70° C. for 18 h. The reaction mixture was evaporated and purified by flash chromatography over silica gel using dichloromethane and methanol. The fractions were evaporated and purified again twice by semi-preparative HPLC. The fractions were collected, extracted with a saturated solution of hydrogen carbonate and ethyl acetate and evaporated to afford, after lyophilization, the expected compound as yellow solid (Yield: 2%, 14 mg). MS m/z (ES) [M+H]⁺: 599.42

### Synthesis of Compound (dq): N-(4-(4-(3-(3-(tert-butyl)-1-phenyl-1H-pyrazol-5-yl)ureido)-3-(methylthio)phenoxy)pyridin-2-yl)-2-hydroxyacetamide

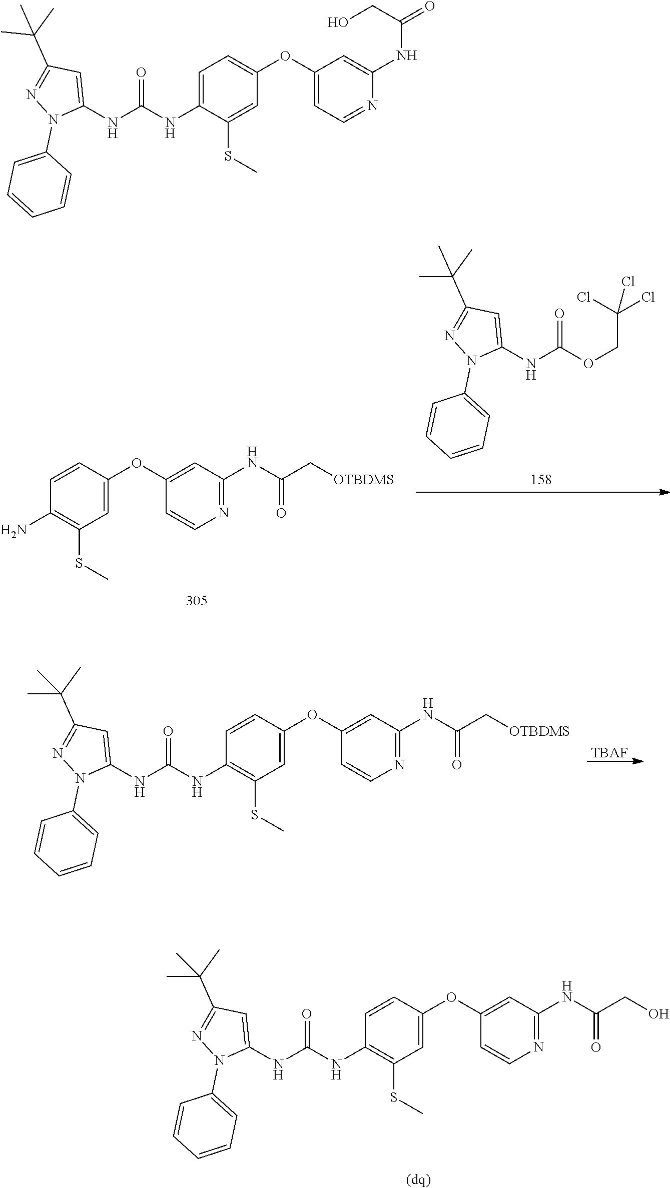 [SMILES: CSc1cc(Oc2ccnc(NC(=O)CO)c2)ccc1NC(=O)Nc1cc(C(C)(C)C)nn1-c1ccccc1 ; CC(C)(C)c1cc(NC(=O)OCC(Cl)(Cl)Cl)n(-c2ccccc2)n1 ; CSc1cc(Oc2ccnc(NC(=O)CO[Si](C)(C)C(C)(C)C)c2)ccc1N ; CSc1cc(Oc2ccnc(NC(=O)CO[Si](C)(C)C(C)(C)C)c2)ccc1NC(=O)Nc1cc(C(C)(C)C)nn1-c1ccccc1 ; "TBAF" ; CSc1cc(Oc2ccnc(NC(=O)CO)c2)ccc1NC(=O)Nc1cc(C(C)(C)C)nn1-c1ccccc1]

### Step1. Synthesis of Compound 1.4

According to Scheme 4 Step 2: In a sealed tube containing N-[4-(4-amino-3-methylsulfanyl-phenoxy)-2-pyridyl]-2-[tert-butyl(dimethyl)silyl]oxy-acetamide 305 (228 mg, 0.54 mmol), DIEA (0.95 ml, 5.43 mmol) in DMSO (0.1M) was added 2,2,2-trichloroethyl N-(5-tert-butyl-2-phenyl-pyrazol-3-yl) carbamate 158 (425 mg, 1.09 mmol). The reaction mixture was stirred at 70° C. for 18 h to furnish, after flash chromatography (40 g, SiO₂, cyclohexane/AcOEt 100:0→60:40, 10 CV) the title compound in mixture with side products (230 mg) as an orange foam.

### Step2. Synthesis of: N-(4-(4-(3-(3-(tert-butyl)-1-phenyl-1H-pyrazol-5-yl)ureido)-3-(methylthio)phenoxy)pyridin-2-yl)-2-hydroxyacetamide (Compound (dq))

A solution of 2-[tert-butyl(dimethyl)silyl]oxy-N-[4-{4-[(5-tert-butyl-2-phenyl-pyrazol-3-yl) carbamoylamino]-3-methylsulfanyl-phenoxy}-2-pyridyl]acetamide (230 mg, 0.35 mmol) and TBAF (1M in THF, 0.21 mL, 0.21 mmol) in dry THF (5 mL) was stirred at room temperature for 4 h. Then water (10 mL) and AcOEt (20 mL) were added. The aqueous layer was extracted with AcOEt (2×20 mL). The combined organic fractions were dried over MgSO₄, filtered and concentrated in vacuo. The crude obtained was purified by flash chromatography (12 g, SiO₂, cyclohexane/AcOEt 100:0→0:100, 10 CV then AcOEt/CH₃OH 100:0→90:10, 5 CV) and semi-preparative HPLC, to furnish the title compound (Yield: 17%, 32 mg) as a white solid. MS m/z (ES) [M+H]⁺: 547.33

¹H NMR (300 MHz, DMSO-d₆) δ 9.74 (s, 1H), 9.00 (s, 1H), 8.37 (s, 1H), 8.17 (d, J=5.7 Hz, 1H), 7.73 (d, J=8.8 Hz, 1H), 6.67 (d, J=2.2 Hz, 1H), 7.53-7.49 (m, 4H), 7.42-7.36 (m, 1H), 7.14 (d, J=2.7 Hz, 1H), 6.97 (dd, J=8.8, 2.7 Hz, 1H), 6.69 (dd, J=5.7, 2.3 Hz, 1H), 6.35 (s, 1H), 5.66 (t, J=6.0 Hz, 1H), 3.98 (d, J=6.0 Hz, 2H), 2.41 (s, 3H), 1.26 (s, 9H). ESIMS m/z [M+H]+ 547.33.

### Example 2: In Vitro Testing

### Example 2.1: Myotube Assay Workflow

DMD human induced pluripotent stem cells (hereafter “DMD hiPSC”), generated from the NORM-1 hiPSC WT cell line by gene editing, or WT human induced pluripotent stem cells (hereafter “WT hiPSC”), being NORM-1 hiPSC wt cell line, have been differentiated according to Chal et al. 2015, 2016, to produce muscle progenitors (hereafter “hMP”). The hMP population contains satellite-like cells (i.e Pax7-positive cells) and satellite cells' progenies (i.e. Myogenin-positive cells).

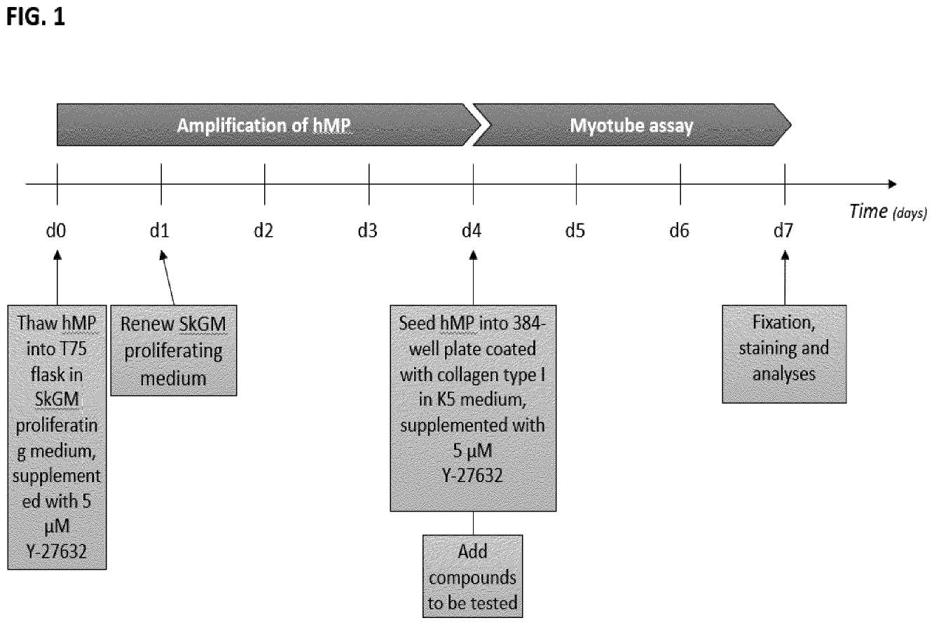

FIG. 1 provides an example of a Myotube assay workflow. At day 0, hMP are thawed and 3.1 million cells are plated into a T75-flask coated with hESC-qualified Matrigel™ (Corning) in SkGM proliferating medium (PromoCells), supplemented with 5 μM Y-27632 (Tocris Bioscience). On day 2, SkGM proliferating medium is refreshed. On day 4, hMP are harvested and transferred into a 384-well plate coated with collagen type I at a density of 3500 cells/well in a DMEM-based medium, containing 5% of knockout serum replacement (Gibco), 1% PenStrep (Gibco), 1% MEM NEAA (Gibco) and 0.1 mM 2-mercaptoethanol (Gibco) (hereafter “K5 medium”) and 5 μM Y-27632. Compounds to be tested are then added in the medium and incubated for 3 days at 37° C. On day 7, cells are fixed and immunostained. For example, myotubes (alpha-actinin staining) and satellite-like (Pax7 staining) cells can be analyzed for total myotube area per well or Pax7-positive cell quantification.

### Example 2.2: Dose Response Assay of Compound (i)

DMD hMP were put in the Myotube assay according to the workflow described in Example 2.1. In particular, compound (i) was added at concentrations ranging from 1.5 nM to 30 μM, starting from day 4. hMP were cultivated for three more days in presence of compound (i), then fixed and analyzed for quantification of total myotube area per well. Duplicates of each condition were averaged.

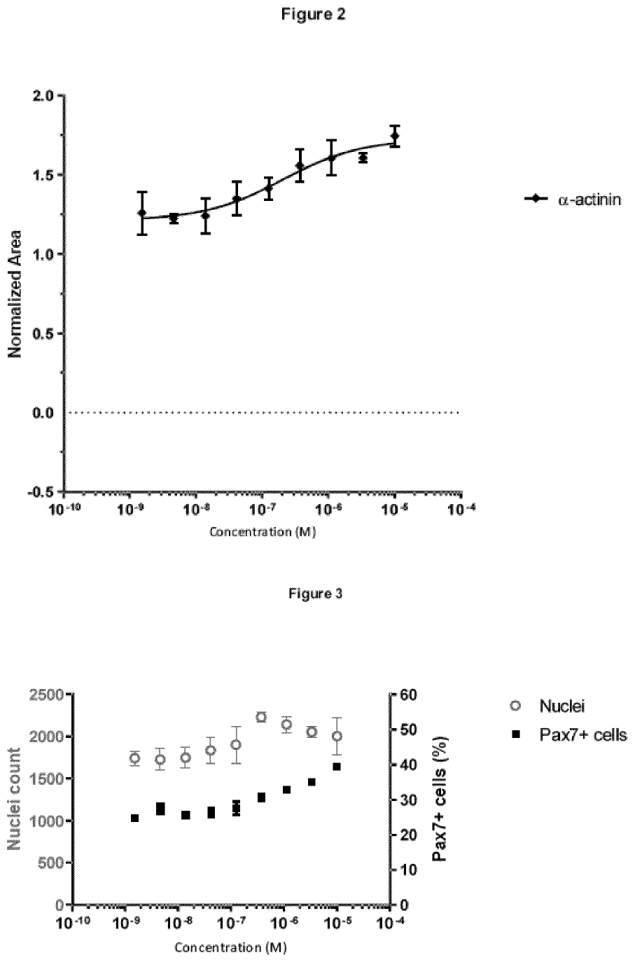

As shown in FIG. 2, treatment of DMD hMP in the myotube assay at 10 different increasing concentrations of compound (i) resulted in dose-dependent increase of myotube surface. This result indicates that compound (i) has a myogenic activity.

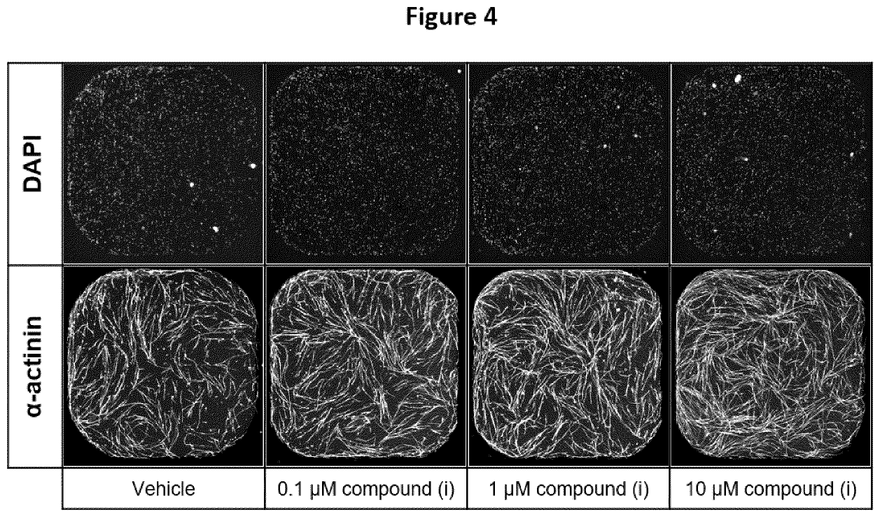

FIG. 4 provides representative images of the dose response experiment shown in FIG. 2.

### Example 2.3: Identification of Compounds that Promote Myogenic Differentiation and Maintain or Increase Satellite-Like Cells

This example describes multiple compounds identified as promoting muscle progenitor differentiation, with the ability to preserve the satellite-like cell pool.

Using the myotube assay described in Example 2.1, all the compounds listed in Table 2, have been shown to be active in the myotube assay.

Table 2 shows myogenic activities (EC₅₀) and efficacies (MA=normalized Myotube Area) of compounds of the present invention. Qualitative scale: ++++: EC₅₀<500 nM; +++: 500 nM≤EC₅₀<1000 nM; ++: 1000 nM≤EC₅₀<5000 nM; +: EC₅₀≥5000 nM; +++: MA≥75%; ++: 50%≤MA<75%; +: 25%≤MA<50%.

TABLE 2

| Compound | EC₅₀ | MA |
|---|---|---|
| (a) | +++ | + |
| (b) | + | + |
| (c) | + | ++ |
| (d) | ++ | ++ |
| (e) | + | + |
| (f) | + | ++ |
| (g) | +++ | +++ |
| (h) | + | + |
| (i) | ++ | ++ |
| (j) | ++ | ++ |
| (k) | ++ | ++ |
| (l) | +++ | ++ |
| (m) | + | ++ |
| (n) | ++++ | +++ |
| (o) | + | + |
| (p) | +++ | + |
| (q) | ++ | ++ |
| (r) | + | +++ |
| (s) | +++ | ++ |
| (t) | ++++ | + |
| (u) | ++ | + |
| (v) | ++++ | +++ |
| (w) | ++ | ++ |
| (x) | + | + |
| (y) | + | + |
| (z) | + | +++ |
| (aa) | ++ | ++ |
| (ab) | +++ | ++ |
| (ac) | ++ | ++ |
| (ad) | +++ | + |
| (ae) | + | ++ |
| (af) | ++ | + |
| (ag) | ++ | +++ |
| (ah) | + | +++ |
| (ai) | +++ | ++ |
| (aj) | +++ | +++ |
| (ak) | + | +++ |
| (al) | + | +++ |
| (am) | ++++ | +++ |
| (an) | ++++ | +++ |
| (ao) | + | +++ |
| (ap) | +++ | + |
| (aq) | +++ | +++ |
| (ar) | + | +++ |
| (as) | ++++ | +++ |
| (at) | + | +++ |
| (au) | + | +++ |
| (av) | + | +++ |
| (aw) | +++ | +++ |
| (ax) | + | ++ |
| (ay) | ++ | +++ |
| (az) | +++ | +++ |
| (ba) | ++++ | +++ |
| (bb) | ++++ | +++ |
| (bc) | ++ | +++ |
| (bd) | ++++ | +++ |
| (be) | +++ | +++ |
| (bf) | ++ | +++ |
| (bg) | + | +++ |
| (bh) | + | +++ |
| (bi) | ++++ | +++ |
| (bj) | + | +++ |
| (bk) | + | +++ |
| (bl) | +++ | ++ |
| (bm) | ++++ | +++ |
| (bn) | + | ++ |
| (bo) | ++++ | +++ |
| (bp) | ++++ | +++ |
| (bq) | ++++ | +++ |
| (br) | + | +++ |
| (bs) | + | +++ |
| (bt) | +++ | +++ |
| (bu) | + | + |
| (bv) | + | +++ |
| (bw) | ++++ | +++ |
| (bx) | +++ | +++ |
| (by) | ++++ | +++ |
| (bz) | ++++ | +++ |
| (ca) | + | +++ |
| (cb) | ++++ | +++ |
| (cc) | ++++ | +++ |
| (cd) | ++++ | +++ |
| (ce) | ++++ | +++ |
| (cf) | +++ | +++ |
| (cg) | + | +++ |
| (ch) | + | ++ |
| (ci) | + | + |
| (cj) | + | +++ |
| (ck) | +++ | +++ |
| (cl) | ++++ | +++ |
| (cm) | +++ | +++ |
| (cn) | ++ | ++ |
| (co) | + | + |
| (cp) | +++ | ++ |
| (cq) | +++ | +++ |
| (cr) | ++++ | +++ |
| (cs) | + | + |
| (ct) | ++++ | +++ |
| (cu) | ++++ | +++ |
| (cv) | ++++ | +++ |
| (cw) | + | +++ |
| (cx) | +++ | +++ |
| (cy) | +++ | +++ |
| (cz) | +++ | +++ |
| (da) | ++++ | +++ |
| (db) | ++++ | +++ |
| (dc) | ++++ | +++ |
| (dd) | ++++ | +++ |
| (de) | ++++ | +++ |
| (df) | ++++ | +++ |
| (dg) | ++++ | +++ |
| (dh) | ++ | +++ |
| (di) | ++++ | +++ |
| (dj) | ++++ | +++ |
| (dk) | + | + |
| (dl) | ++ | +++ |
| (dm) | ++ | +++ |
| (dn) | +++ | +++ |
| (do) | ++++ | +++ |
| (dp) | ++++ | +++ |
| (dq) | +++ | +++ |

### Example 2.4: Compounds Activity on Satellite Cell (PAX7+) Subpopulation In Vitro

DMD hMP were put in the Myotube assay according to the workflow described in Example 2.1. In particular, compound (i) was added at concentrations ranging from 1.5 nM to 30 μM, starting from day 4. hMP were cultivated for three more days in presence of compound (i), then fixed and analyzed for percentage of Pax7-positive cells and nuclei number. Duplicates of each condition were averaged.

As shown in FIG. 3, treatment of DMD hMP in the myotube assay at 10 different increasing concentrations of compound (i) resulted in dose-dependent increase percentage of Pax7-positive cells. This result indicates that compound (i) at least maintains the pool of satellite-like cells.

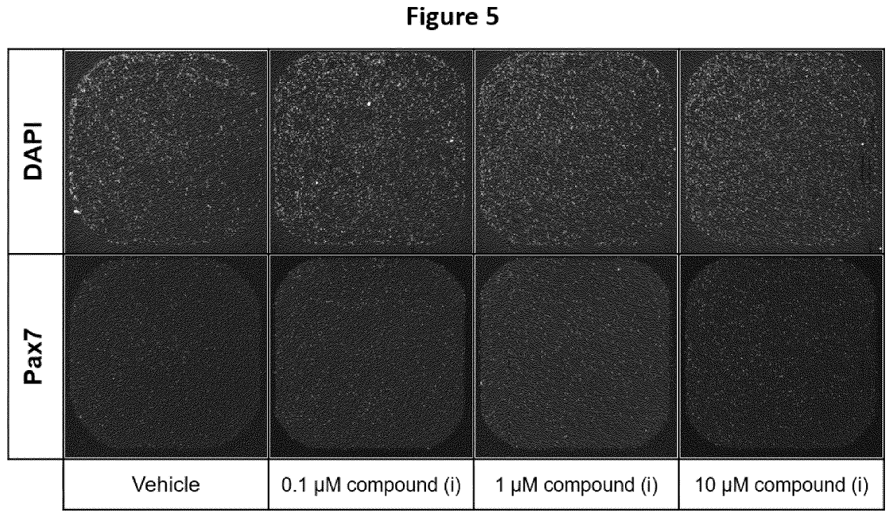

FIG. 5 provides representative images of the dose response experiment shown in FIG. 3.

This example describes multiple compounds identified as promoting muscle progenitor differentiation, with the ability to preserve the satellite-like cell pool.

Using the assay for measuring Pax7-positive cells described in Example 2.1, almost all the compounds listed in Table 3, have been shown to be capable to maintain or increase the pool of Pax7-positive cells in the assay.

The results are presented in Table 3. (−): Depletes the pool of cells, while positively affects the myotubes; (+): Maintains or increases the pool of cells, with at least 1 of the highest concentration known to be cytotoxic; and (++) Maintains or increases the pool of cells, without any toxicity.

TABLE 3

| Compound | Pax7% |
|---|---|
| (a) | ++ |
| (b) | ++ |
| (c) | + |
| (d) | + |
| (e) | ++ |
| (f) | ++ |
| (g) | + |
| (h) | + |
| (i) | + |
| (j) | ++ |
| (k) | ++ |
| (l) | + |
| (m) | ++ |
| (n) | + |
| (o) | ++ |
| (p) | ++ |
| (q) | ++ |
| (r) | ++ |
| (s) | + |
| (t) | + |
| (U) | ++ |
| (v) | ++ |
| (w) | ++ |
| (x) | ++ |
| (y) | ++ |
| (z) | ++ |
| (aa) | ++ |
| (ab) | ++ |
| (ac) | ++ |
| (ad) | ++ |
| (ae) | + |
| (af) | ++ |
| (ag) | ++ |
| (ah) | ++ |
| (ai) | ++ |
| (aj) | ++ |
| (ak) | + |
| (al) | ++ |
| (am) | ++ |
| (an) | ++ |
| (ao) | ++ |
| (ap) | ++ |
| (aq) | + |
| (ar) | ++ |
| (as) | + |
| (at) | ++ |
| (au) | ++ |
| (av) | ++ |
| (aw) | + |
| (ax) | ++ |
| (ay) | ++ |
| (az) | ++ |
| (ba) | ++ |
| (bb) | + |
| (bc) | ++ |
| (bd) | + |
| (be) | ++ |
| (bf) | + |
| (bg) | ++ |
| (bh) | ++ |
| (bi) | ++ |
| (bj) | ++ |
| (bk) | ++ |
| (bl) | + |
| (bm) | + |
| (bn) | ++ |
| (bo) | + |
| (bp) | + |
| (bq) | + |
| (br) | ++ |
| (bs) | ++ |
| (bt) | ++ |
| (bu) | ++ |
| (bv) | ++ |
| (bw) | ++ |
| (bx) | ++ |
| (by) | + |
| (bz) | + |
| (ca) | ++ |
| (cb) | ++ |
| (cc) | + |
| (cd) | ++ |
| (ce) | + |
| (cf) | − |
| (cg) | − |
| (ch) | + |
| (ci) | ++ |
| (cj) | + |
| (ck) | + |
| (cl) | + |
| (cm) | + |
| (cn) | ++ |
| (co) | ++ |
| (cp) | + |
| (cq) | ++ |
| (cr) | + |
| (cs) | + |
| (ct) | + |
| (cu) | ++ |
| (cv) | + |
| (cw) | ++ |
| (cx) | + |
| (cy) | + |
| (cz) | + |
| (da) | + |
| (db) | + |
| (dc) | + |
| (dd) | + |
| (de) | + |
| (df) | + |
| (dg) | + |
| (dh) | + |
| (di) | ++ |
| (dj) | + |
| (dk) | ++ |
| (dl) | ++ |
| (dm) | − |
| (dn) | + |
| (do) | + |
| (dp) | + |
| (dq) | + |

### Example 2.5: Comparative Examples

Other compounds have been tested in the same assays. The results are presented in Table 4.

TABLE 4

| Cpd name | EC₅₀ | MA | Pax7 |
|---|---|---|---|
| Rebastinib (FIG. 18 of WO2019/084499) | ++ | ++++ | + |
| CCT196969 (FIG. 18 of WO2019/084499) | +++ | ++ | + |
| CEP-32496 (FIG. 18 of WO2019/084499) | NA | NA | n.a. |
| P38a MAPK-IN-1 (FIG. 18 of WO2019/084499) | + | + | ++ |
| BIRB-796 (FIG. 18 of WO2019/084499) | + | +++ | ++ |
| Sorafenib (FIG. 18 of WO2019/084499) | + | + | ++ |
| Cabozantinib (FIG. 18 of WO2019/084499) | NA | NA | n.a. |
| Foretinib (FIG. 18 of WO2019/084499) | NA | NA | n.a. |

NA: non active

1-59. (canceled)

60. A method of treating or ameliorating a disease or condition associated with muscle cells and/or satellite cells comprising the administration of a compound or a pharmaceutically acceptable salt thereof to an individual with said disease or condition, wherein said compound is represented by structure (I), 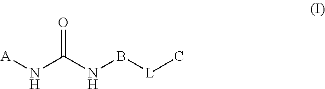
wherein
L is —O—
and
1) A is a ring system selected from the group consisting of 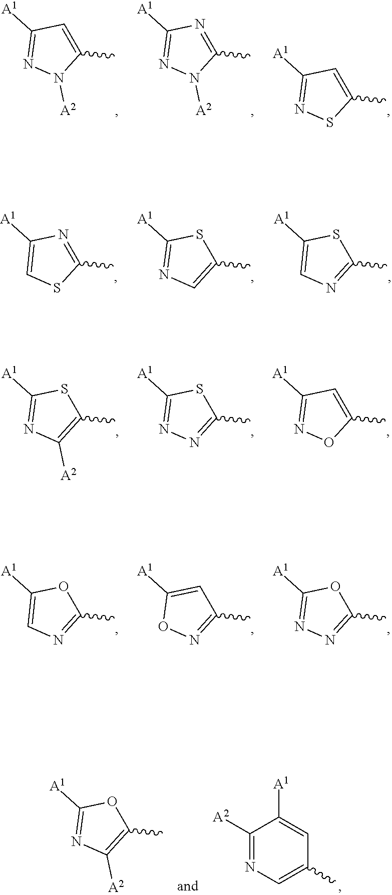
wherein A¹ is selected from the group consisting of C₁-C₄ alkyl, C₁-C₄ cycloalkyl, 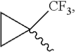
C₃-C₇ heterocycloalkyl, 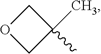
phenyl, benzyl and 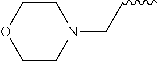
wherein A¹ is optionally fluorinated,
and A² is selected from the group consisting of —H, C₁-C₄ alkyl, C₃-C₆ cycloalkyl, C₃-C₇ heterocycloalkyl, halogen, benzyl, phenyl, tolyl, 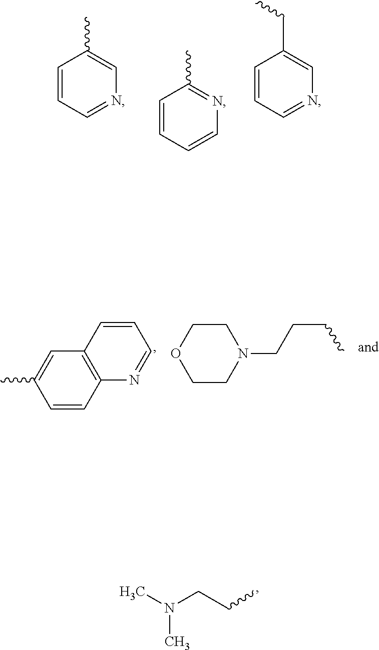
wherein A¹ and A² are optionally fluorinated, and A₂ is optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,
B is a ring system selected from the group consisting of 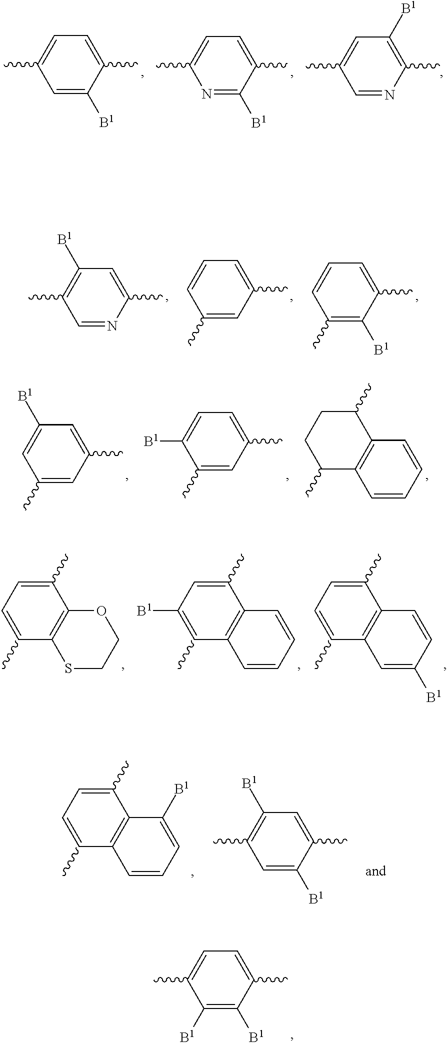
wherein B¹ is selected from the group consisting of —H, —F, —Cl, —SCH₃, —SCH₂CH₃, —CH₃, isopropyl, —CF₃, —OCH₃, —OCF₃ and wherein two B¹ can be linked to form a fused bicyclic ring system containing 0 to 3 heteroatoms; and wherein B¹ is not H when B is a phenyl;
C is selected from the group consisting of 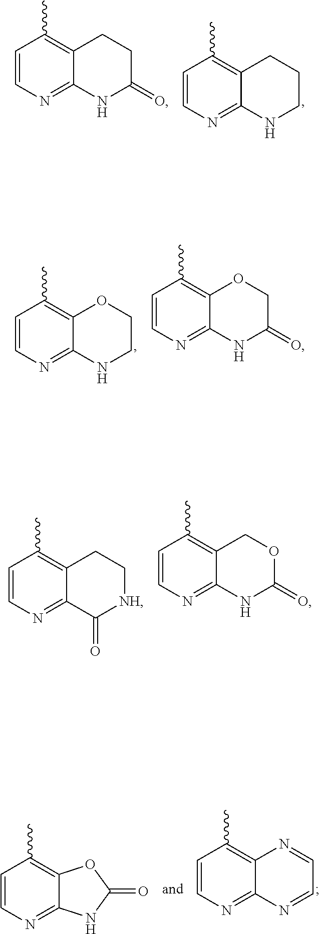
or
2) A is a ring system selected from the group consisting of 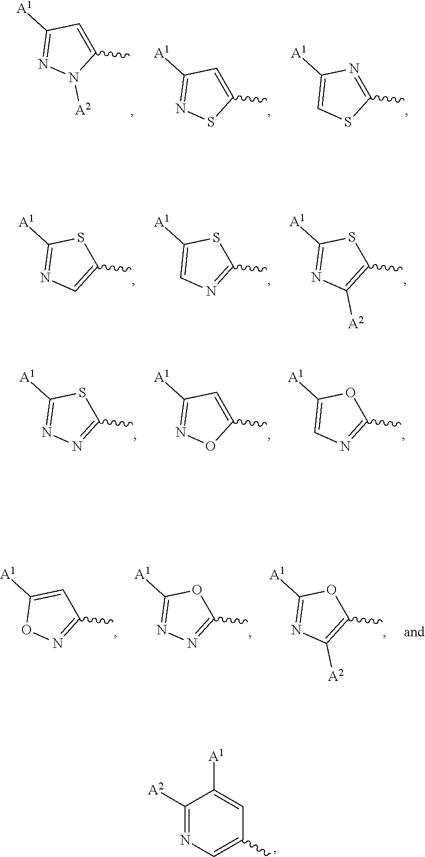
wherein A¹ is selected from the group consisting of C₁-C₄ alkyl, C₁-C₄ cycloalkyl, 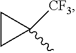
C₃-C₇ heterocycloalkyl, 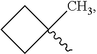
phenyl, benzyl and 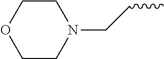
wherein A¹ is optionally fluorinated,
and A² is selected from the group consisting of —H, C₁-C₄ alkyl, C₃-C₆ cycloalkyl, C₃-C₇ heterocycloalkyl, halogen, benzyl, phenyl, tolyl, 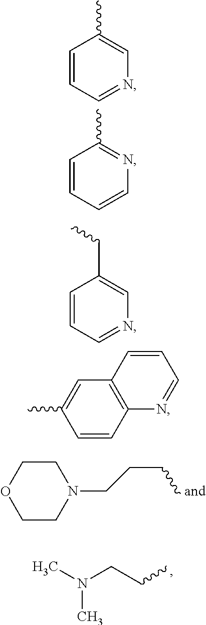
wherein A¹ and A² are optionally fluorinated, and A₂ is optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,
B is a ring system selected from the group consisting of 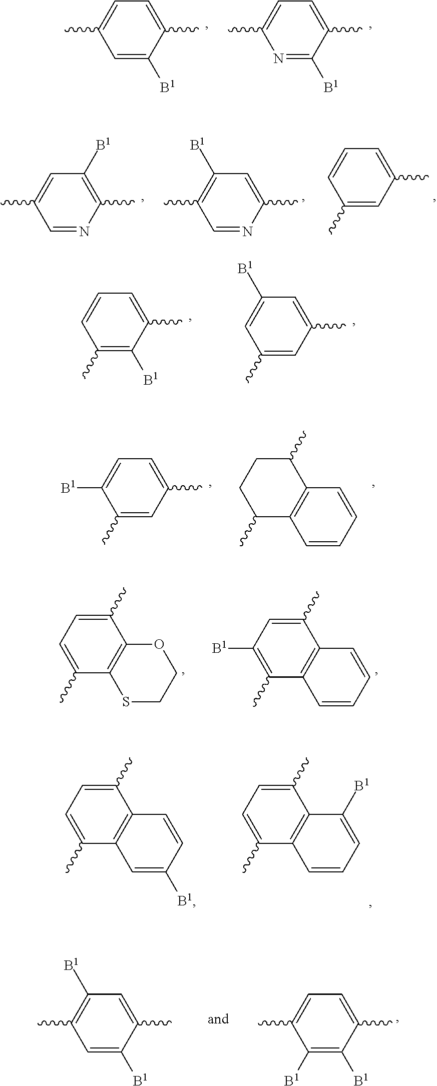
wherein B¹ is selected from the group consisting of —H, —F, —SCH₃, —SCH₂CH₃, —CH₃, isopropyl, —CF₃, —OCH₃, —OCF₃ and wherein two B¹ can be linked to form a fused bicyclic ring system containing 0 to 3 heteroatoms; and wherein B¹ is not H when B is a phenyl;
C is selected from the group consisting of 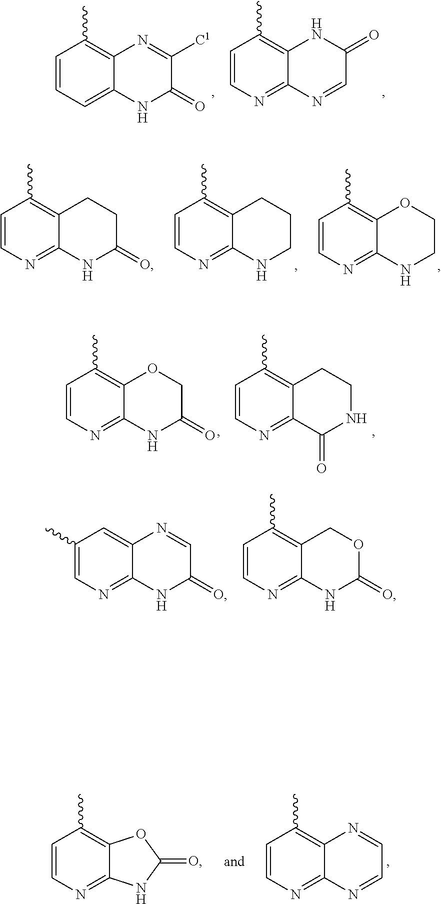
wherein C¹ is selected from the group consisting of —H, —CH₃, —OCH₃, —CN or 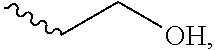
or C is selected from the group consisting of 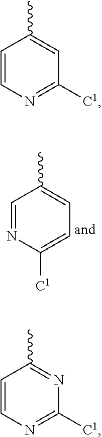
wherein C¹ is selected from the group consisting of wherein C¹ is selected from the group consisting of —H, —NH₂, —NR¹, —C(O)—NH—R¹, —NH—C(O)—R¹, —NH—S(O)₂—R¹, —NH—C(O)—OR¹, —C(O)—R¹, —R¹, —OR¹, —SO₂R¹, with R¹ being selected from the group consisting of C₁-C₁₀ alkyl, C₂-C₁₀ alkenyl, C₃-C₁₀ cycloalkyl, C₄-C₂₀ alkcycloalkyl, C₆-C₁₄ aryl, C₃-C₁₃ heteroaryl, C₇-C₂₄ alkaryl, C₃-C₇ heterocycloalkyl and C₄-C₁₇ alkheterocycloalkyl, wherein each hydrogen in each of these groups may be independently replaced by a group selected from halogen, hydroxy, —OR′, —COR′, —COOR′, and —NR′R″, each hydrogen in each of R′ and R″ may be independently replaced by —COR′″ or COOR′″, wherein R′, R″ and R′″ are independently a C₁-C₆ alkyl;
or
3) A is a ring system selected from the group consisting of 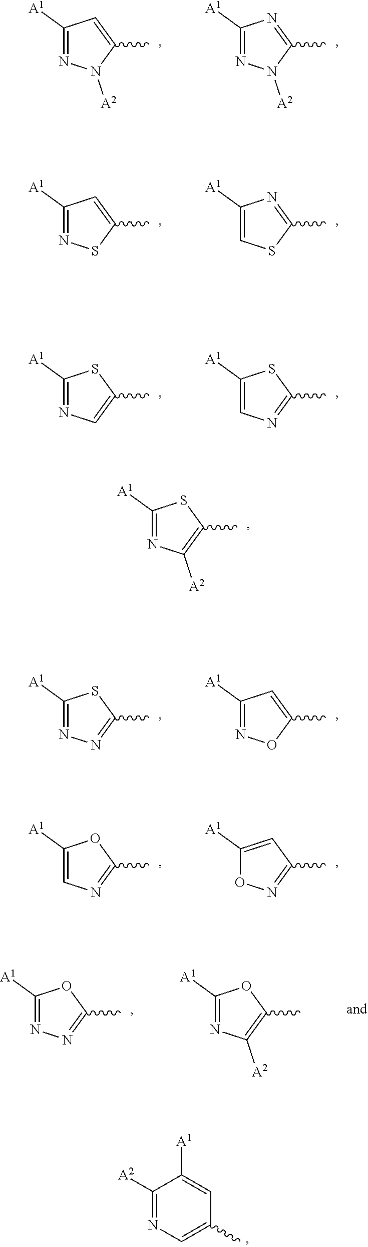
wherein A¹ is selected from the group consisting of C₁-C₄ alkyl, C₁-C₄ cycloalkyl, 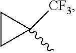
C₃-C₇ heterocycloalkyl, 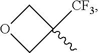
phenyl, benzyl and 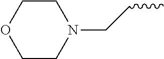
wherein A¹ is optionally fluorinated,
and A² is selected from the group consisting of —H, C₁-C₄ alkyl, C₃-C₆ cycloalkyl, C₃-C₇ heterocycloalkyl, halogen, benzyl, phenyl, tolyl, 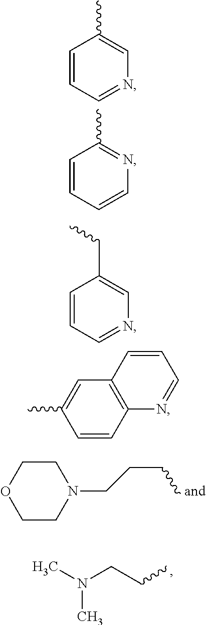
wherein A¹ and A² are optionally fluorinated, and A₂ is optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,
B is a ring system selected from the group consisting of 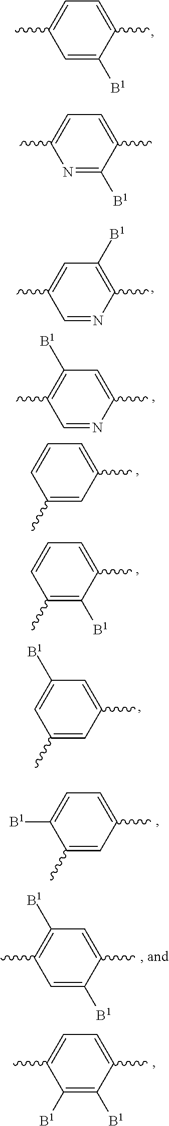
wherein B¹ is selected from the group consisting of —CF₃, —OCF₃, —OCH₃, and SCH₂CH₃, or B has two B¹ groups selected independently from the group consisting of —F, —Cl, —SCH₃, —SCH₂CH₃, —CH₃, isopropyl, —CF₃, —OCH₃, —OCF₃,
C is selected from the group consisting of 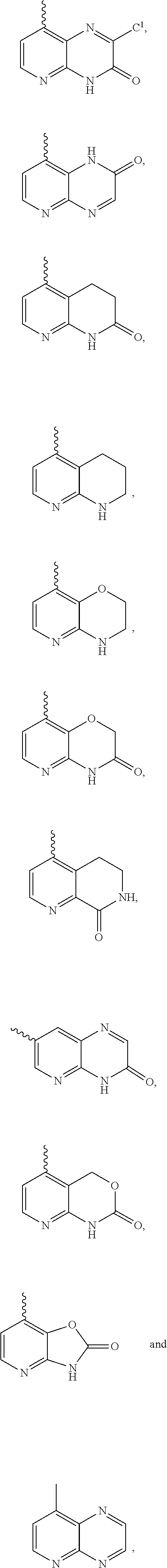
wherein C¹ is selected from the group consisting of —H, —CH₃, —OCH₃, —CN or 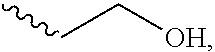
or C is selected from the group consisting of 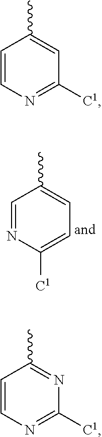
wherein C¹ is selected from the group consisting of wherein C¹ is selected from the group consisting of —H, —NH₂, —NR¹, —C(O)—NH—R¹, —NH—C(O)—R¹, —NH—S(O)₂—R¹, —NH—C(O)—OR¹, —C(O)—R¹, —R¹, —OR¹, —SO₂R¹, with R¹ being selected from the group consisting of C₁-C₁₀ alkyl, C₂-C₁₀ alkenyl, C₃-C₁₀ cycloalkyl, C₄-C₂₀ alkcycloalkyl, C₆-C₁₄ aryl, C₃-C₁₃ heteroaryl, C₇-C₂₄ alkaryl, C₃-C₇ heterocycloalkyl and C₄-C₁₇ alkheterocycloalkyl, wherein each hydrogen in each of these groups may be independently replaced by a group selected from halogen, hydroxy, —OR′, —COR′, —COOR′, and —NR′R″, each hydrogen in each of R′ and R″ may be independently replaced by —COR′″ or COOR′″, wherein R′, R″ and R′″ are independently a C₁-C₆ alkyl;
or
4) A is 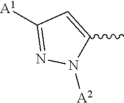
wherein A¹ is selected from the group consisting of C₁-C₄ alkyl, C₁-C₄ cycloalkyl, 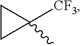
C₃-C₇ heterocycloalkyl, 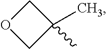
phenyl, benzyl and 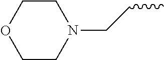
wherein A¹ is optionally fluorinated,
and A² is selected from the group consisting of a phenyl substituted with 1, 2 or 3 substituents selected from the group consisting of —Cl, —F, —CN, C₁-C₃ alkyloxy optionally substituted by C₃-C₇ heterocycloalkyl or NRR, with R being a C₁-C₃ alkyl, and C₁-C₄ alkyl substituted by C₃-C₇ heterocycloalkyl or NRR, with R being a C₁-C₃ alkyl; a phenyl or pyridinyl substituted by 2 or 3 C₁-C₃ alkyls; and a pyridinyl substituted with 1, 2 or 3 substituents selected from the group consisting of —Cl, —F, —CN, C₁-C₁₀ alkyloxy substituted by C₃-C₇ heterocycloalkyl or NRR, with R being a C₁-C₃ alkyl, and C₁-C₄ alkyl substituted by C₃-C₇ heterocycloalkyl or NRR, with R being a C₁-C₃ alkyl;
B is a ring system selected from the group consisting of 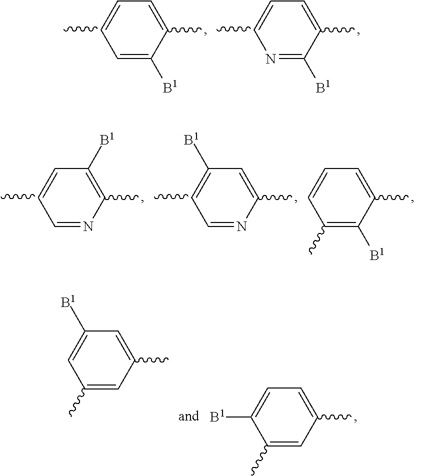
wherein B¹ is —SCH₃,
C is selected from the group consisting of 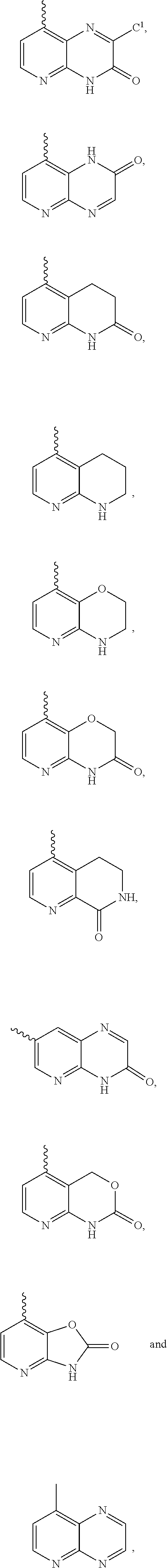
wherein C¹ is selected from the group consisting of —H, —CH₃, —OCH₃, —CN or 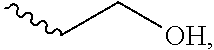
or
5) A is 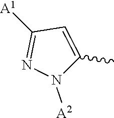
and
A¹ is selected from the group consisting of C₁-C₄ alkyl, C₃-C₆ cycloalkyl, 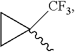
C₃-C₇ heterocycloalkyl, 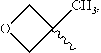
phenyl, benzyl and 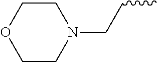
wherein A¹ is optionally fluorinated,
A¹ is 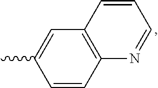
B is a ring system selected from the group consisting of 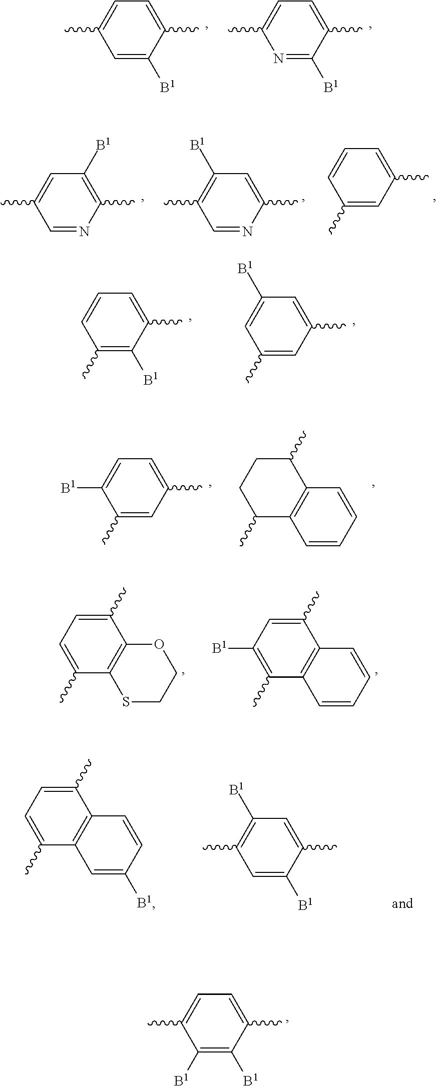
wherein B¹ is selected from the group consisting of —H, —F, —Cl, —SCH₃, —SCH₂CH₃, —CH₃, isopropyl, —CF₃, —OCH₃, —OCF₃ and wherein two B¹ can be linked to form a fused bicyclic ring system containing 0 to 3 heteroatoms; and wherein B¹ is not H when B is a phenyl;
C is selected from the group consisting of 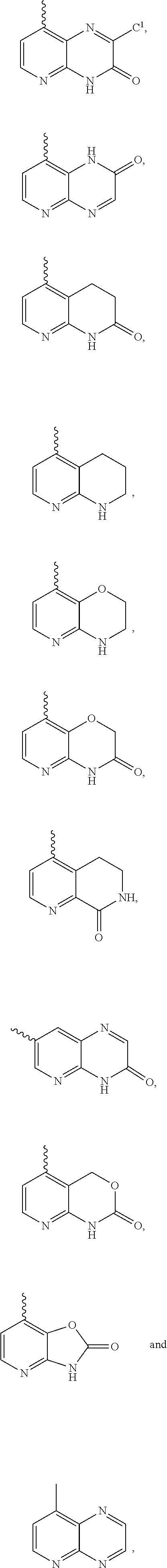
wherein C¹ is selected from the group consisting of —H, —CH₃, —OCH₃, —CN or 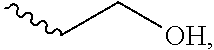
or
6) A is a ring system selected from the group consisting of 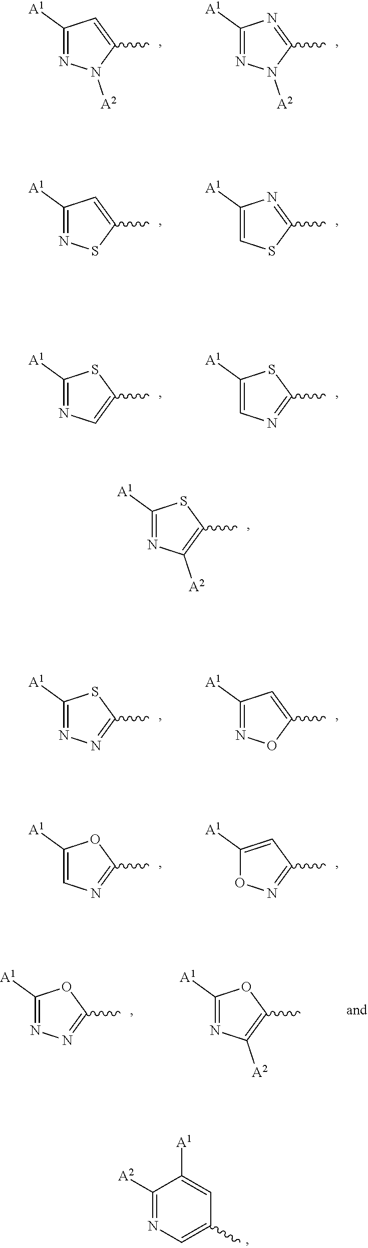
wherein A¹ is selected from the group consisting of C₁-C₄ alkyl, C₁-C₄ cycloalkyl, 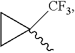
C₃-C₇ heterocycloalkyl, 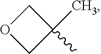
phenyl, benzyl and 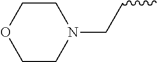
wherein A¹ is optionally fluorinated,
and A² is selected from the group consisting of —H, C₁-C₄ alkyl, C₃-C₆ cycloalkyl, C₃-C₇ heterocycloalkyl, halogen, benzyl, phenyl, tolyl, 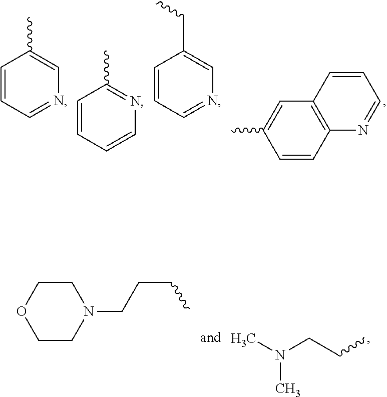
wherein A¹ and A² are optionally fluorinated, and A₂ is optionally substituted with 1, 2 or 3 substituents selected from the group consisting of C₁-C₄ alkyl, —Cl, —F, —CN, —OR^{AB}c, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,
B is a ring system selected from the group consisting of 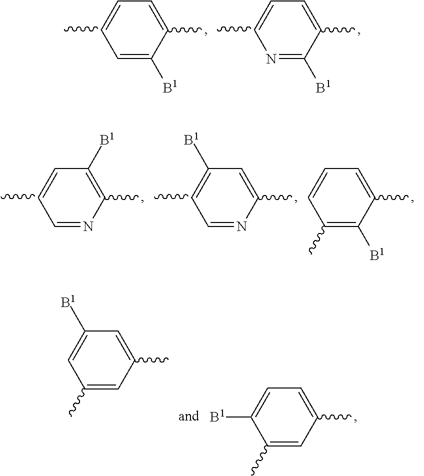
wherein B¹ is —SCH₃,
C is selected from the group consisting of 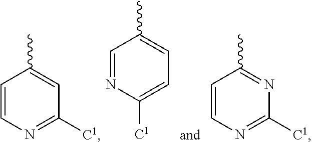
wherein C¹ is selected from the group consisting of wherein C¹ is selected from the group consisting of —H, —NH₂, —NR¹, —C(O)—NH—R¹, —NH—C(O)—R¹, —NH—S(O)₂—R¹, —NH—C(O)—OR¹, —C(O)—R¹, —R¹, —OR¹, —SO₂R¹, with R¹ being selected from the group consisting of C₁-C₁₀ alkyl, C₂-C₁₀ alkenyl, C₃-C₁₀ cycloalkyl, C₄-C₂₀ alkcycloalkyl, C₆-C₁₄ aryl, C₃-C₁₃ heteroaryl, C₇-C₂₄ alkaryl, C₃-C₇ heterocycloalkyl and C₄-C₁₇ alkheterocycloalkyl, wherein each hydrogen in each of these groups may be independently replaced by a group selected from halogen, hydroxy, —OR′, —COR′, —COOR′, and —NR′R″, each hydrogen in each of R′ and R″ may be independently replaced by —COR′″ or COOR′″, wherein R′, R″ and R′″ are independently a C₁-C₆ alkyl;
or
7) A is a ring system selected from the group consisting of 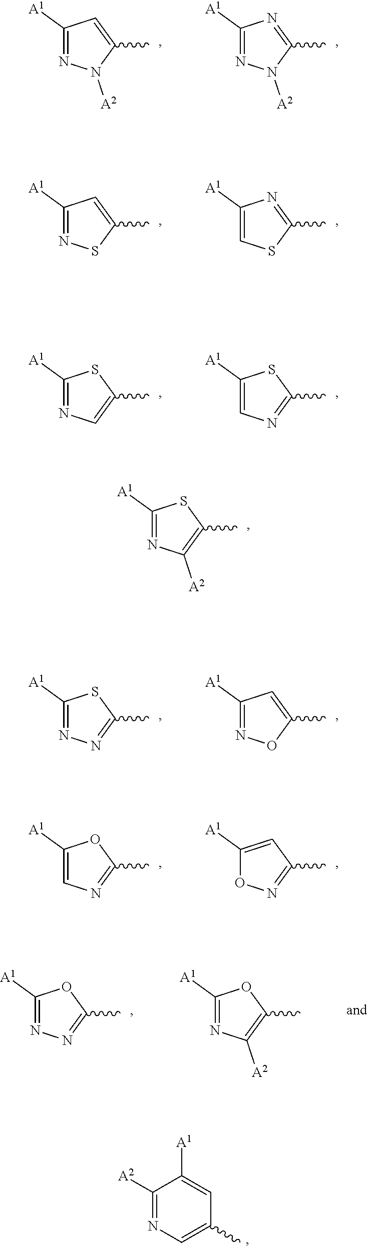
wherein A¹ is selected from the group consisting of C₁-C₄ alkyl, C₁-C₄ cycloalkyl, 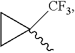
C₃-C₇ heterocycloalkyl, 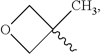
phenyl, benzyl and 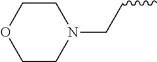
wherein A¹ is optionally fluorinated,
and A² is selected from the group consisting of —H, C₁-C₄ alkyl, C₃-C₆ cycloalkyl, C₃-C₇ heterocycloalkyl, halogen, benzyl, phenyl, tolyl, 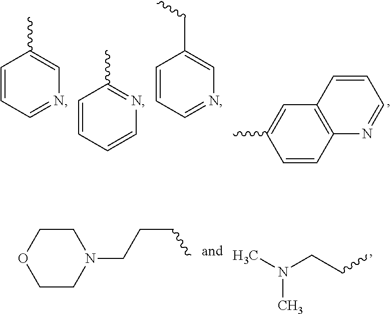
wherein A¹ and A² are optionally fluorinated, and A₂ is optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,
B is a ring system selected from the group consisting of 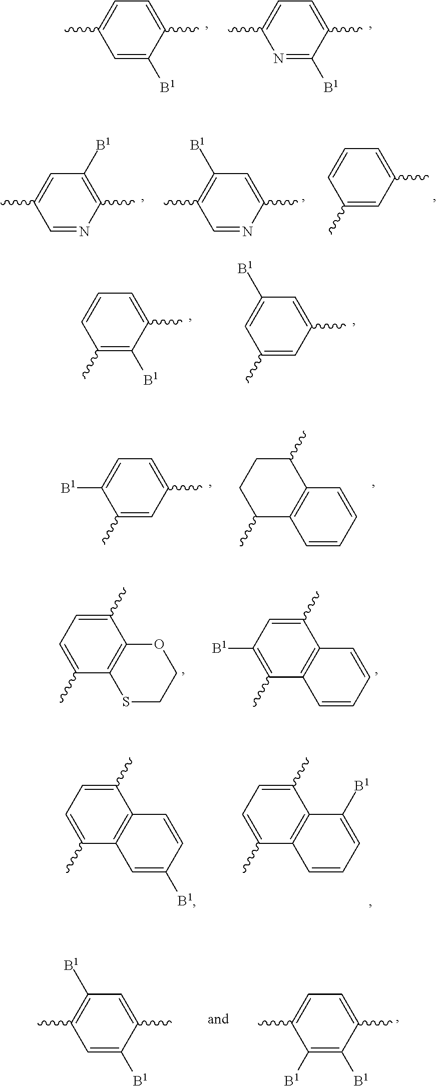
wherein B¹ is selected from the group consisting of —H, —F, —Cl, —SCH₃, —SCH₂CH₃, —CH₃, isopropyl, —CF₃, —OCH₃, —OCF₃ and wherein two B¹ can be linked to form a fused bicyclic ring system containing 0 to 3 heteroatoms; and wherein B¹ is not H when B is a phenyl;
C is selected from the group consisting of 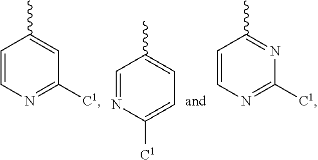
wherein C¹ is selected from the group consisting of —NH—C(O)R¹ or —NH—C(O)—OR¹ with R¹ being selected from the group consisting of C₁-C₁₀ alkyl, C₆-C₁₄ aryl, C₃-C₁₃ heteroaryl, and C₇-C₂₄ alkaryl, wherein each hydrogen in each of these groups may be independently replaced by a group selected from halogen, hydroxy, —OR′, and —NR′R″, each hydrogen in each of R′ and R″ may be independently replaced by —COR′″ or COOR′″, wherein R′, R″ and R′″ are independently a C₁-C₆ alkyl.

61. The method according to claim 60,
wherein A is a ring system selected from the group consisting of 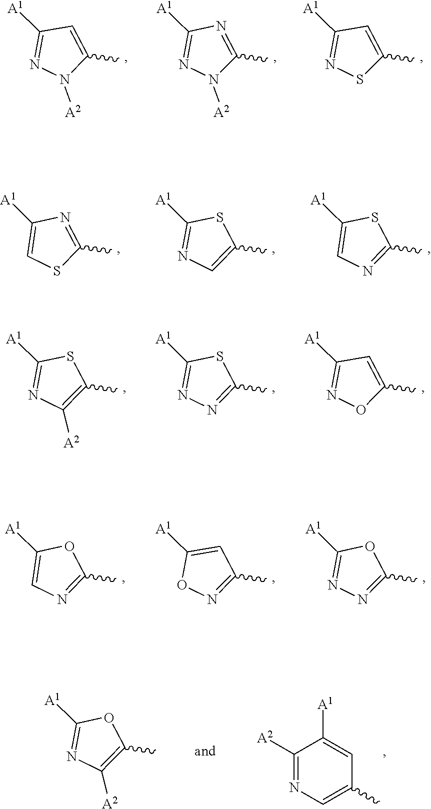
A¹ is selected from the group consisting of methyl, —CF₃, isopropyl, cyclopropyl, isobutyl, sec-butyl, tert-butyl, 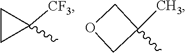
and A² is selected from the group consisting of H, methyl, iso-propyl, benzyl, phenyl, chlorine, 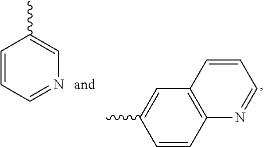
wherein A¹ and A² are optionally fluorinated, and A₂ is optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,
B is a ring system selected from the group consisting of 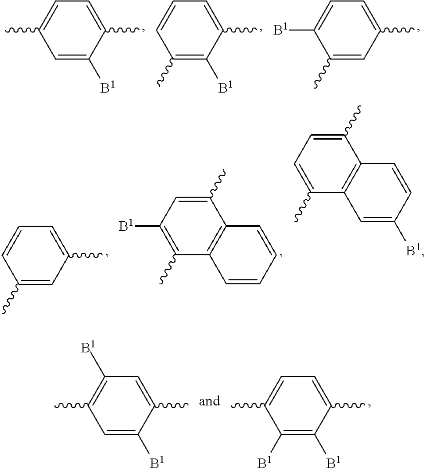
wherein, when B is a phenyl, B¹ is selected from the group consisting of —F, —Cl, —SCH₃, —SCH₂CH₃, —CH₃, —CF₃, —OCH₃, and —OCF₃ and, when B is a naphtyl, B¹ is —H, and
C is selected from the group consisting of 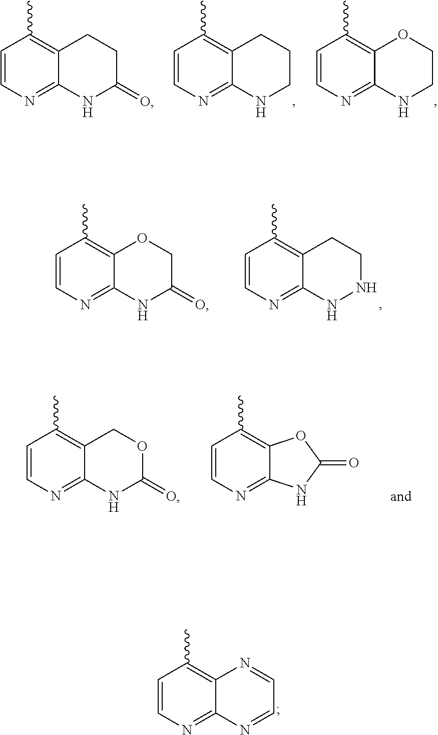
or
wherein A is a ring selected from the group consisting of 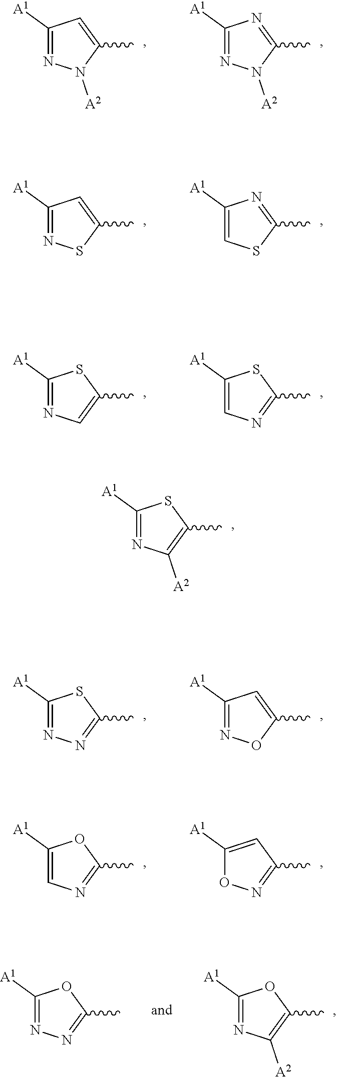
A¹ is tert-butyl,
A² is a phenyl or 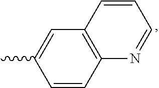
optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,
B is a ring system selected from the group consisting of 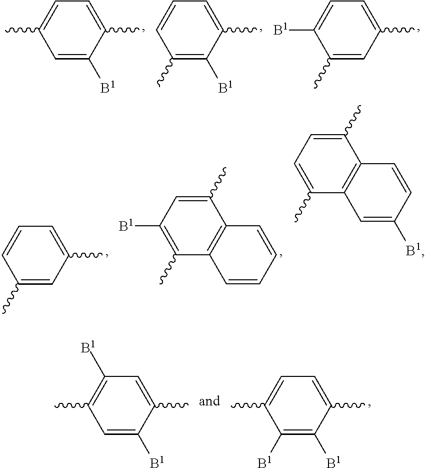
wherein, when B is a phenyl, B¹ is selected from the group consisting of —F, —SCH₃, —SCH₂CH₃, —CF₃, —OCH₃, and —OCF₃ and, when B is a naphtyl, B¹ is —H, and
C is selected from the group consisting of 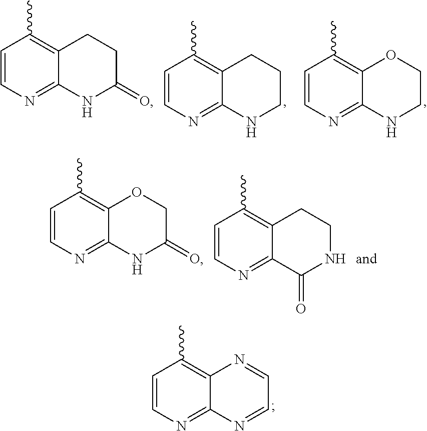
or
wherein A is a ring selected from the group consisting of 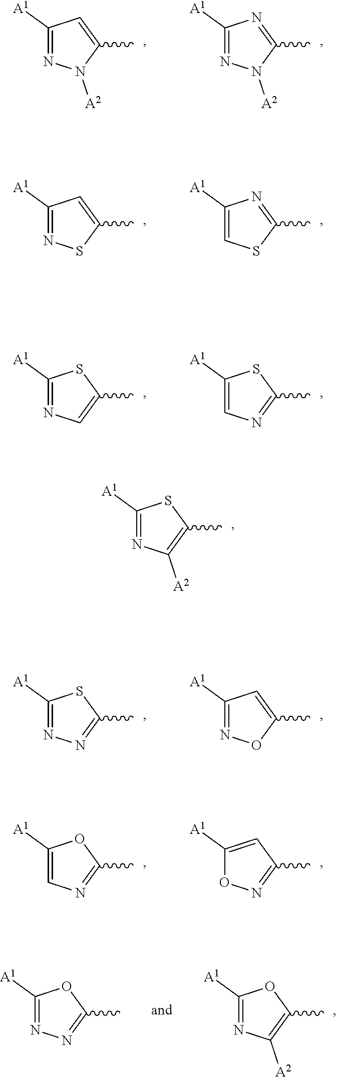
A¹ is tert-butyl,
A² is a phenyl or 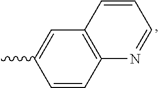
optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,
B is a ring system selected from the group consisting of 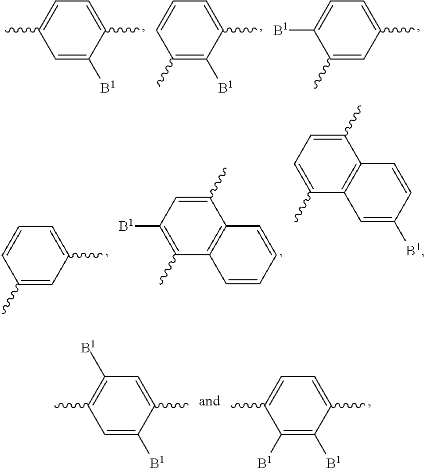
wherein, when B is a phenyl, B¹ is selected from the group consisting of —F, —SCH₃, —SCH₂CH₃, —CF₃, —OCH₃, and —OCF₃ and, when B is a naphtyl, B¹ is —H, and
C is selected from the group consisting of 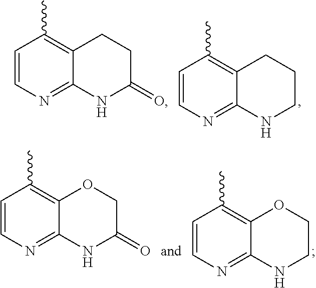
or
wherein A is a ring selected from the group consisting of 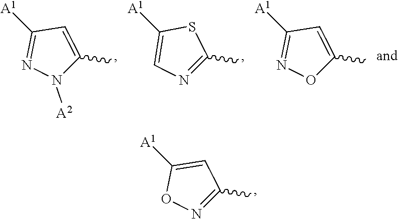
A¹ is tert-butyl,
A² is a phenyl optionally substituted with 1, 2 or 3 substituents selected from the group consisting of methyl, —Cl, —F, —CN, methoxy, —O—(CH₂)₂-piperidinyl, —O—(CH₂)₂-morpholinyl, —O—(CH₂)₃—N(CH₃)₂, and —CH₂-morpholinyl, or A² is 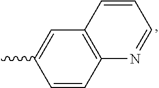
B is a ring system selected in the group consisting of 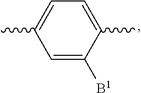
with B¹ being selected from the group consisting of —F, —SCH₃, —SCH₂CH₃, —CF₃, —OCH₃, and —OCF₃, preferably —SCH₃, 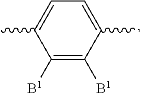
with B¹ being F,
and 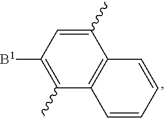
with B¹ being —H, and
C is selected from the group consisting of

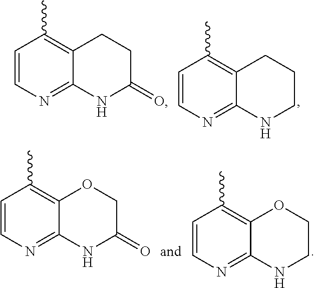

62. The method according to claim 60, wherein the compound is selected in the group consisting of
1-(3-(tert-butyl)-1-phenyl-1H-pyrazol-5-yl)-3-(2-(methylthio)-4-((7-oxo-5,6,7,8-tetrahydro-1,8-naphthyridin-4-yl)oxy)phenyl)urea;
1-(3-(tert-butyl)-1-phenyl-1H-pyrazol-5-yl)-3-(2-(methylthio)-4-((5,6,7,8-tetrahydro-1,8-naphthyridin-4-yl)oxy)phenyl)urea;
1-(3-(tert-butyl)-1-(p-tolyl)-1H-pyrazol-5-yl)-3-(2-(methylthio)-4-((7-oxo-5,6,7,8-tetrahydro-1,8-naphthyridin-4-yl)oxy)phenyl)urea;
1-(5-(tert-butyl)thiazol-2-yl)-3-(2-(methylthio)-4-((7-oxo-5,6,7,8-tetrahydro-1,8-naphthyridin-4-yl)oxy)phenyl)urea;
1-(3-(tert-butyl)-1-phenyl-1H-pyrazol-5-yl)-3-(2-(methylthio)-4-((3-oxo-3,4-dihydro-2H-pyrido[3,2-b][1,4]oxazin-8-yl)oxy)phenyl)urea;
1-(5-(tert-butyl)thiazol-2-yl)-3-(4-((7-oxo-5,6,7,8-tetrahydro-1,8-naphthyridin-4-yl)oxy)naphthalen-1-yl)urea;
1-(3-(tert-butyl)-1-phenyl-1H-pyrazol-5-yl)-3-(4-((3,4-dihydro-2H-pyrido[3,2-b][1,4]oxazin-8-yl)oxy)-2-(methylthio)phenyl)urea;
1-(3-(tert-butyl)isoxazol-5-yl)-3-(2-(methylthio)-4-((7-oxo-5,6,7,8-tetrahydro-1,8-naphthyridin-4-yl)oxy)phenyl)urea;
1-(3-(tert-butyl)isoxazol-5-yl)-3-(4-((7-oxo-5,6,7,8-tetrahydro-1,8-naphthyridin-4-yl)oxy)naphthalen-1-yl)urea; and
1-(3-(tert-butyl)-1-phenyl-1H-pyrazol-5-yl)-3-(2-(methylthio)-4-((7-oxo-7,8-dihydro-1,8-naphthyridin-4-yl)oxy)phenyl)urea
or a pharmaceutically acceptable salt thereof.

63. The method according to claim 60,
wherein A is a ring selected from the group consisting of 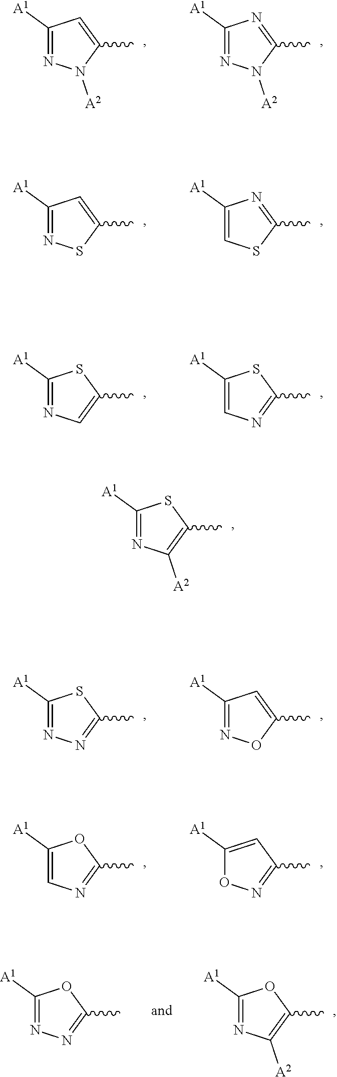
A¹ is selected from the group consisting of methyl, —CF₃, isopropyl, cyclopropyl, isobutyl, sec-butyl, tert-butyl, 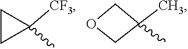
A² is —H,
B is a ring system selected from the group consisting of 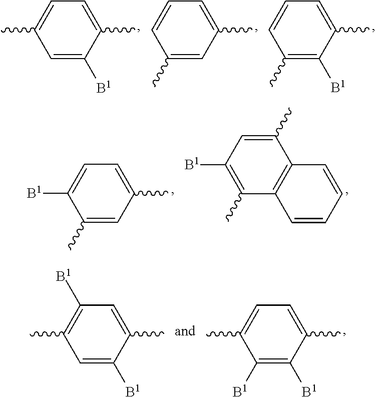
wherein, when B is a phenyl, B¹ is selected from the group consisting of —F, —Cl, —SCH₃, —SCH₂CH₃, —CH₃, —CF₃, —OCH₃, and —OCF₃ and, when B is a naphtyl, B¹ is —H, and
C is selected from the group consisting of 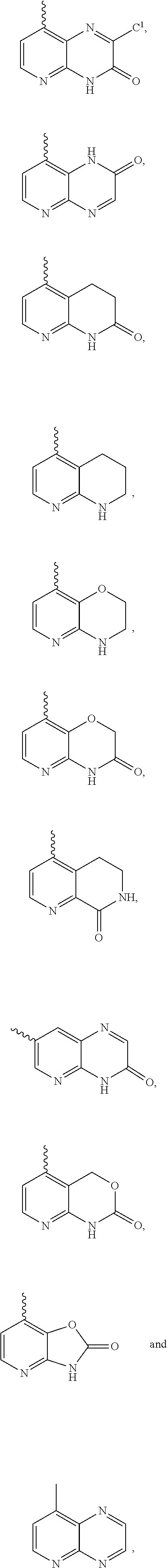
wherein C¹ is selected from the group consisting of —H, —CH₃, —OCH₃, —CN or 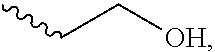
or
C is selected from the group consisting of 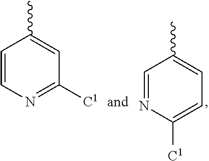
with C¹ being selected C¹ is selected from the group consisting of wherein C¹ is selected from the group consisting of —H, —NH₂, —NR¹, —C(O)—NH—R¹, —NH—C(O)—R¹, —NH—S(O)₂—R¹, —NH—C(O)—OR¹, —C(O)—R¹, —R¹, —OR¹, —SO₂R¹, with R¹ being selected from the group consisting of C₁-C₁₀ alkyl, C₆-C₁₄ aryl, C₃-C₁₃ heteroaryl, and C₇-C₂₄ alkaryl, wherein each hydrogen in each of these groups may be independently replaced by a group selected from halogen, hydroxy, —OR′, and —NR′R″, each hydrogen in each of R′ and R″ may be independently replaced by —COR′″ or COOR′″, wherein R′, R″ and R′″ are independently a C₁-C₆ alkyl;
or
wherein A is a ring selected from the group consisting of 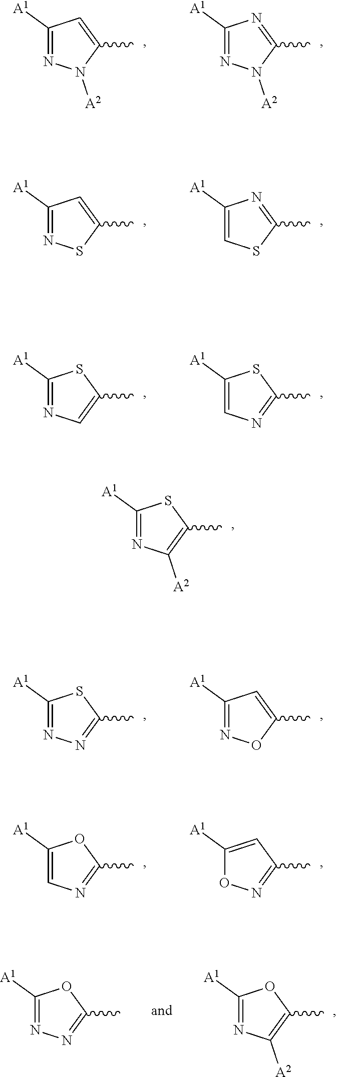
A¹ is tert-butyl,
A² is —H,
B is a ring system selected from the group consisting of 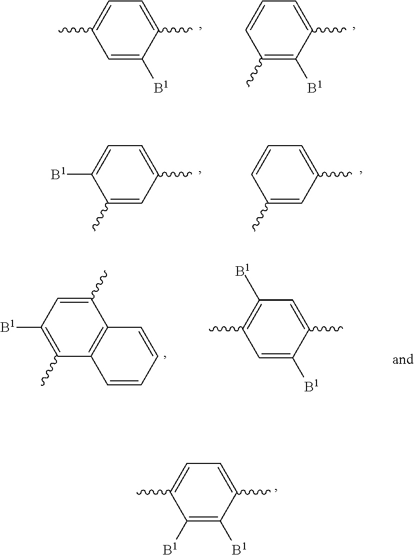
wherein, when B is a phenyl, B¹ is selected from the group consisting of —F, —SCH₃, —SCH₂CH₃, —CF₃, —OCH₃, and —OCF₃ and, when B is a naphtyl, B¹ is —H, and
C is selected from the group consisting of 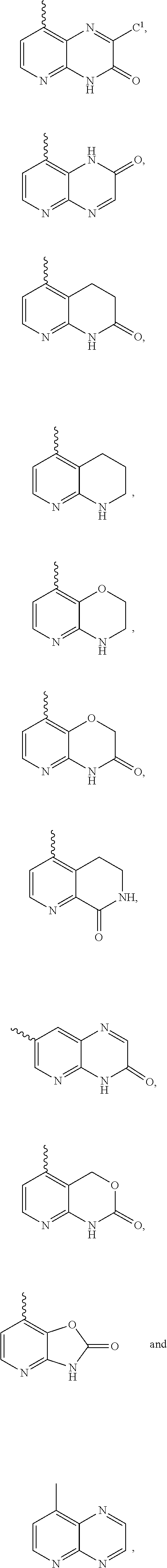
wherein C¹ is selected from the group consisting of —H, —CH₃, —OCH₃, —CN or 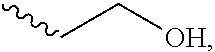
or
C is selected from the group consisting of 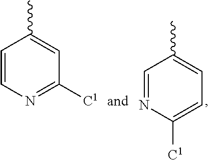
with C¹ being selected from the group consisting of —H, —NH₂, 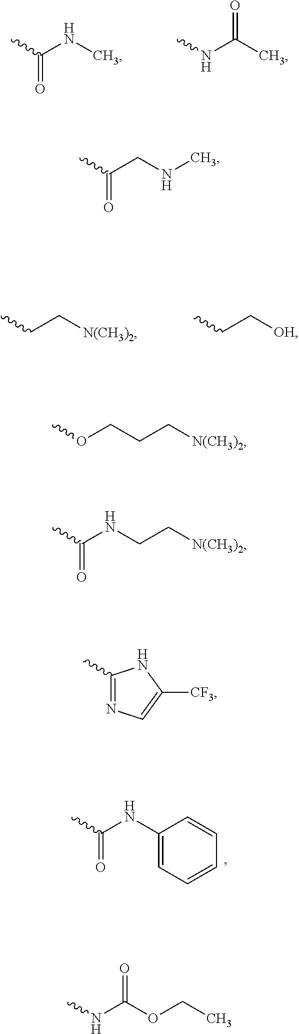
—NH—C(O)OtBu, —NH—C(O)N(CH3)-C(O)OtBu, —NH—C(O)—CH₂—N(CH₃)—C(O)OtBu, —NH—C(O)—CH₂—NHCH₃, —NH—C(O)—Obenzyl, —NH—C(O)—CH₂—OH, —NH—(CH₂)₂—N(CH₃)₂, and —NH—S(O)₂—CH₃, preferably from the group consisting of —H, 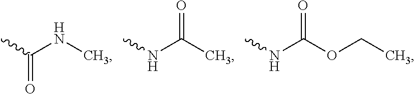
—NH—C(O)OtBu, —NH—C(O)N(CH₃)—C(O)OtBu, —NH—C(O)—CH₂—N(CH₃)—C(O)OtBu, —NH—C(O)—CH₂—NHCH₃, —NH—C(O)-Obenzyl, and —NH—C(O)—CH₂—OH;
or
wherein A is selected in the group consisting of 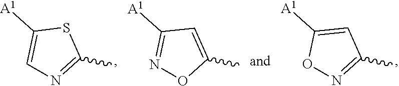
A¹ is tert-butyl,
B is a ring system selected in the group consisting of 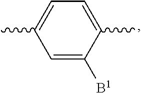
with B¹ being selected from the group consisting of —F, —SCH₃, —SCH₂CH₃, —CF₃, —OCH₃, and —OCF₃, preferably —SCH₃, 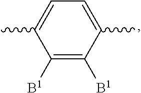
with B¹ being F,
and 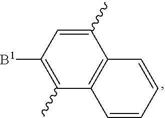
with B¹ being —H, and
C is selected in the group consisting of 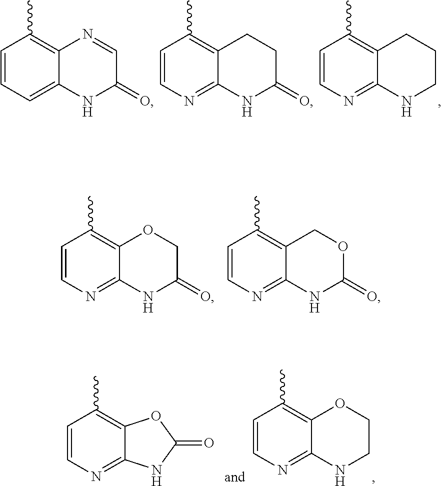
or C is selected from the group consisting of 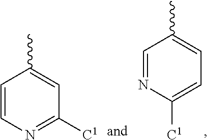
with C¹ being selected from the group consisting of 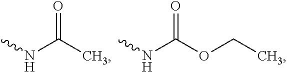
—NH—C(O)OtBu, —NH—C(O)N(CH3)-COOtBu and —NH—C(O)—CH₂—N(CH₃)—C(O)OtBu.

64. The method according to claim 60, wherein the compound is selected in the group consisting of
1-(5-(tert-butyl)thiazol-2-yl)-3-(4-((3-oxo-3,4-dihydropyrido[2,3-b]pyrazin-8-yl)oxy)naphthalen-1-yl)urea;
1-(3-(tert-butyl)isoxazol-5-yl)-3-(2-(methylthio)-4-((3-oxo-3,4-dihydropyrido[2,3-b]pyrazin-8-yl)oxy)phenyl)urea;
1-(5-(tert-butyl)thiazol-2-yl)-3-(2-(methylthio)-4-((7-oxo-5,6,7,8-tetrahydro-1,8-naphthyridin-4-yl)oxy)phenyl)urea;
1-(5-(tert-butyl)thiazol-2-yl)-3-(4-((7-oxo-5,6,7,8-tetrahydro-1,8-naphthyridin-4-yl)oxy)naphthalen-1-yl)urea;
1-(3-(tert-butyl)isoxazol-5-yl)-3-(2-(methylthio)-4-((7-oxo-5,6,7,8-tetrahydro-1,8-naphthyridin-4-yl)oxy)phenyl)urea;
ethyl (4-(4-(3-(3-(tert-butyl)isoxazol-5-yl)ureido)-3-(methylthio)phenoxy)pyridin-2-yl)carbamate;
ethyl (4-(4-(3-(5-(tert-butyl)thiazol-2-yl)ureido)-3-(methylthio)phenoxy)pyridin-2-yl)carbamate;
1-(3-(tert-butyl)isoxazol-5-yl)-3-(4-((7-oxo-5,6,7,8-tetrahydro-1,8-naphthyridin-4-yl)oxy)naphthalen-1-yl)urea; and
ethyl (4-(4-(3-(5-(tert-butyl)isoxazol-3-yl)ureido)-3-(methylthio)phenoxy)pyridin-2-yl)carbamate;
or a pharmaceutically acceptable salt thereof.

65. The method according to claim 60,
wherein A is a ring system selected from the group consisting of 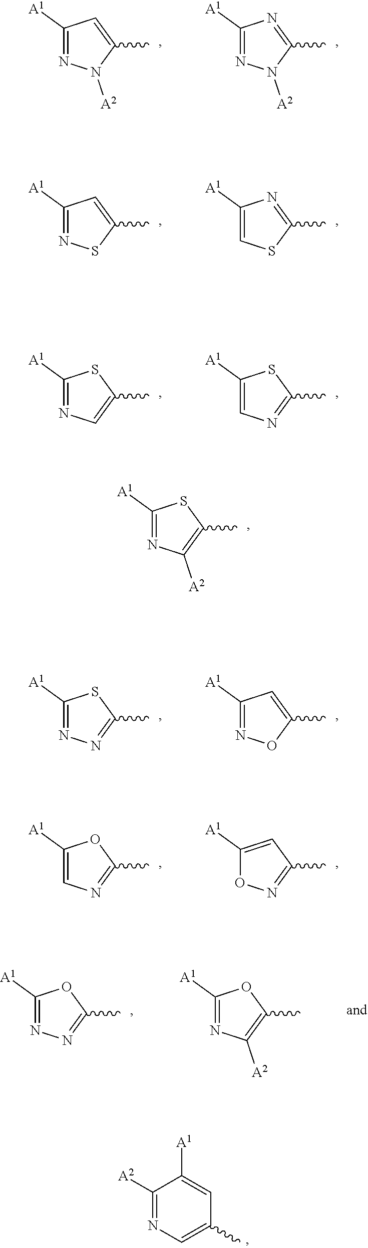
A¹ is selected from the group consisting of methyl, —CF₃, isopropyl, cyclopropyl, isobutyl, sec-butyl, tert-butyl, 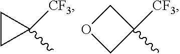
and A² is selected from the group consisting of H, methyl, iso-propyl, benzyl, phenyl, chlorine, 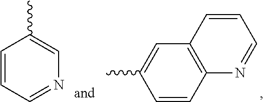
wherein A¹ and A² are optionally fluorinated, and A₂ is optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,
B is a ring system selected from the group consisting of 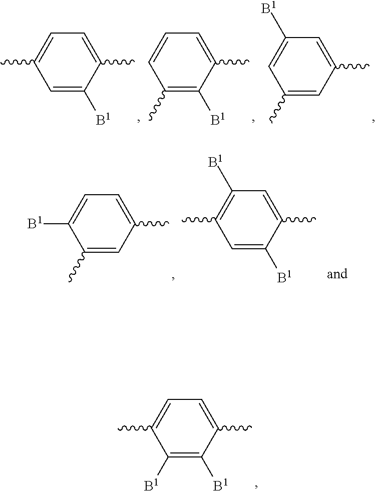
wherein B¹ is selected from the group consisting of —CF₃, —OCH₃, and SCH₂CH₃, or B has two B¹ groups selected independently from the group consisting of —F, —Cl, —SCH₃, —SCH₂CH₃, —CH₃, isopropyl, —CF₃, —OCH₃,
C is selected from the group consisting of 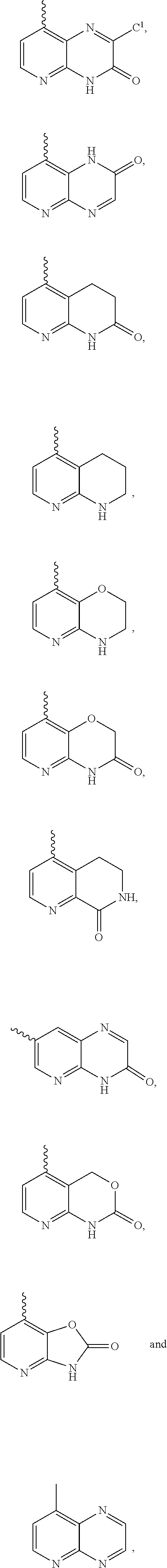
wherein C¹ is selected from the group consisting of —H, —CH₃, —OCH₃, —CN or 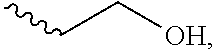
or
C is selected from the group consisting of 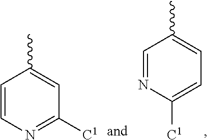
with C¹ being selected C¹ is selected from the group consisting of wherein C¹ is selected from the group consisting of —H, —NH₂, —NR¹, —C(O)—NH—R¹, —NH—C(O)—R¹, —NH—S(O)₂—R¹, —NH—C(O)—OR¹, —C(O)—R¹, —R¹, —OR¹, —SO₂R¹, with R¹ being selected from the group consisting of C₁-C₁₀ alkyl, C₆-C₁₄ aryl, C₃-C₁₃ heteroaryl, and C₇-C₂₄ alkaryl, wherein each hydrogen in each of these groups may be independently replaced by a group selected from a halogen, a hydroxy, —OR′, and —NR′R″, each hydrogen in each of R′ and R″ may be independently replaced by —COR′″ or COOR′″, wherein R′, R″ and R′″ are independently a C₁-C₆ alkyl;
or
wherein A is a ring selected from the group consisting of 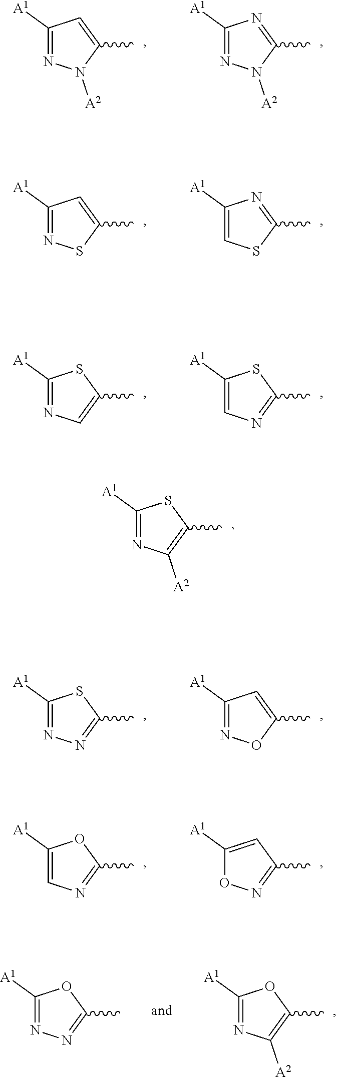
A¹ is tert-butyl,
A² is a phenyl or 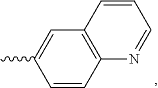
optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,
B is a ring system selected from the group consisting of 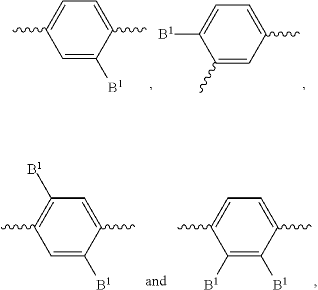
wherein B¹ is selected from the group consisting of —CF₃, —OCF₃, —OCH₃, and SCH₂CH₃, or B has two B¹ groups selected independently from the group consisting of —F, —Cl, —SCH₃, —SCH₂CH₃, —CH₃, isopropyl, —CF₃, —OCH₃, —OCF₃,
C is selected from the group consisting of 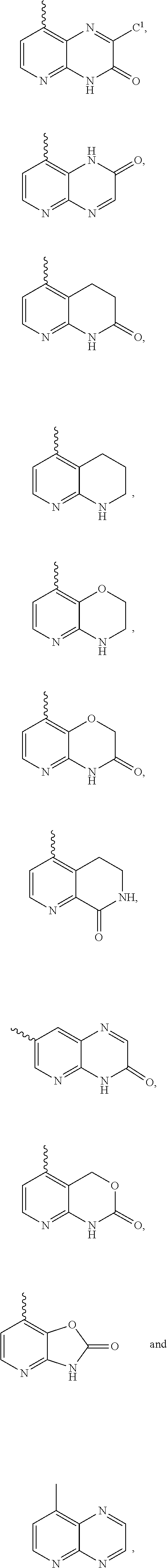
wherein C¹ is selected from the group consisting of —H, —CH₃, —OCH₃, —CN or 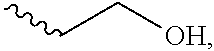
or
C is selected from the group consisting of 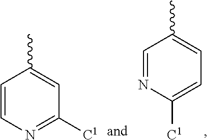
with C¹ being selected from the group consisting of —H, —NH₂, 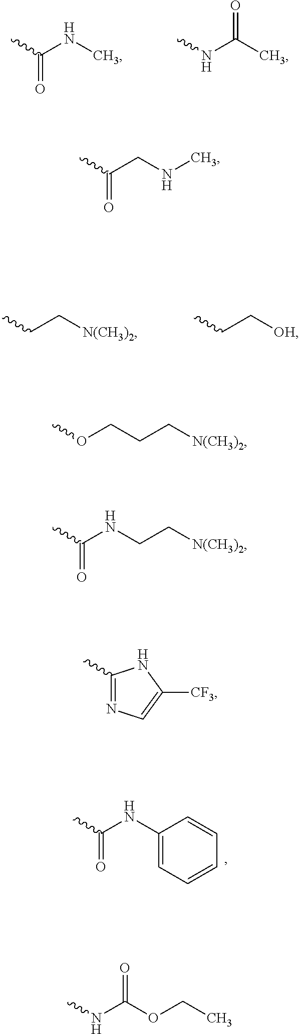
—NH—C(O)OtBu, —NH—C(O)N(CH3)-C(O)OtBu, —NH—C(O)—CH₂—N(CH₃)—C(O)OtBu, —NH—C(O)—CH₂—NHCH₃, —NH—C(O)—Obenzyl, —NH—C(O)—CH₂—OH, —NH—(CH₂)₂—N(CH₃)₂, and —NH—S(O)₂—CH₃, preferably from the group consisting of —H, 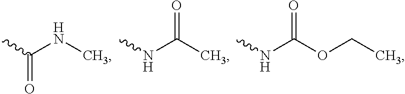
—NH—C(O)OtBu, —NH—C(O)N(CH₃)—C(O)OtBu, —NH—C(O)—CH₂—N(CH₃)—C(O)OtBu, —NH—C(O)—CH₂—NHCH₃, —NH—C(O)—Obenzyl, and —NH—C(O)—CH₂—OH,
or
wherein A is a ring selected from the group consisting of 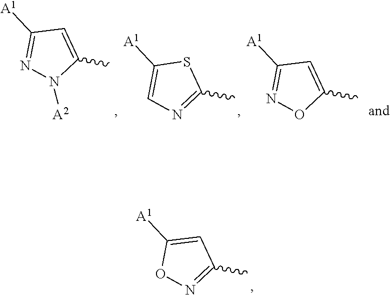
A¹ is tert-butyl,
A² is a phenyl optionally substituted with 1, 2 or 3 substituents selected from the group consisting of methyl, —Cl, —F, —CN, methoxy, —O—(CH₂)₂-piperidinyl, —O—(CH₂)₂-morpholinyl, —O—(CH₂)₃—N(CH₃)₂, and —CH₂-morpholinyl, or A² is 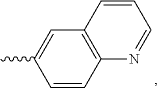
B is a ring system selected in the group consisting of 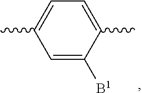
with B¹ being selected from the group consisting of —CF₃, —SCH₂CH₃, —OCH₃, and —OCF₃, preferably —CF₃ or —SCH₂CH₃, 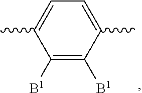
with B¹ being F,
C is selected in the group consisting of 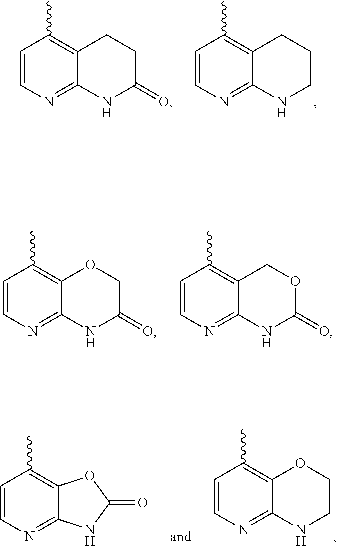
or C is selected from the group consisting of 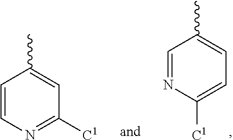
with C¹ being selected from the group consisting of 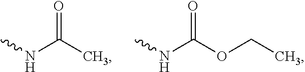
—NH—C(O)OtBu, —NH—C(O)—CH₂—N(CH₃)—C(O)OtBu and —NH—C(O)N(CH3)-COOtBu.

66. The method according to claim 60, wherein the compound is selected in the group consisting of
1-(3-(tert-butyl)-1-phenyl-1H-pyrazol-5-yl)-3-(4-((3-oxo-3,4-dihydropyrido[2,3-b]pyrazin-8-yl)oxy)-2-(trifluoromethyl)phenyl)urea;
1-(3-(tert-butyl)-1-(p-tolyl)-1H-pyrazol-5-yl)-3-(4-((3-oxo-3,4-dihydropyrido[2,3-b]pyrazin-8-yl)oxy)-2-(trifluoromethyl)phenyl)urea;
1-(3-(tert-butyl)-1-phenyl-1H-pyrazol-5-yl)-3-(2-(ethylthio)-4-((3-oxo-3,4-dihydropyrido[2,3-b]pyrazin-8-yl)oxy)phenyl)urea;
1-(3-(tert-butyl)-1-phenyl-1H-pyrazol-5-yl)-3-(2,3-difluoro-4-((3-oxo-3,4-dihydropyrido[2,3-b]pyrazin-8-yl)oxy)phenyl)urea; and
1-(3-(tert-butyl)-1-phenyl-1H-pyrazol-5-yl)-3-(2,5-difluoro-4-((3-oxo-3,4-dihydropyrido[2,3-b]pyrazin-8-yl)oxy)phenyl)urea;
or a pharmaceutically acceptable salt thereof.

67. The method according to claim 60,
wherein A is 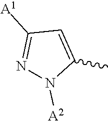
A¹ is selected from the group consisting of methyl, —CF₃, isopropyl, cyclopropyl, isobutyl, sec-butyl, tert-butyl, 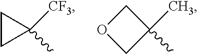
and A² is selected from the group consisting of a phenyl substituted with 1, 2 or 3 substituents selected from the group consisting of methoxy, —Cl, —F, —CN, —O—(CH₂)₂-piperidinyl, —O—(CH₂)₂-morpholinyl, —O—(CH₂)₃—N(CH₃)₂, and —CH₂-morpholinyl; a phenyl or pyridinyl substituted with 2 or 3 methyls, and a pyridinyl substituted with 1, 2 or 3 substituents selected from the group consisting of methyl, —Cl, —F, —CN, —O—(CH₂)₂-piperidinyl, —O—(CH₂)₂-morpholinyl, —O—(CH₂)₃—N(CH₃)₂, and —CH₂-morpholinyl;
B is a ring system selected from the group consisting of 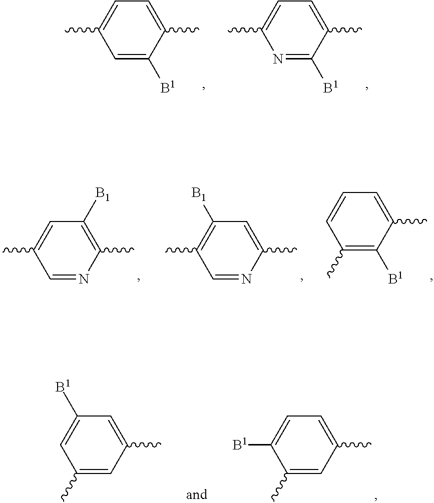
wherein B¹ is —SCH₃,
C is selected from the group consisting of 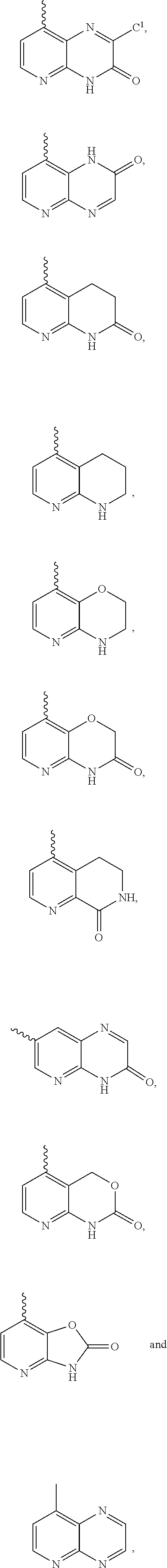
wherein C¹ is selected from the group consisting of —H, —CH₃, —OCH₃, —CN or 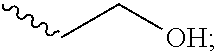
or
wherein A is 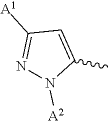
A¹ is tert-butyl,
and A² is selected from the group consisting of a phenyl substituted with 1, 2 or 3 substituents selected from the group consisting of methoxy, —Cl, —F, —CN, —O—(CH₂)₂-piperidinyl, —O—(CH₂)₂-morpholinyl, —O—(CH₂)₃—N(CH₃)₂, and —CH₂-morpholinyl; a phenyl or pyridinyl substituted with 2 or 3 methyls, and a pyridinyl substituted with 1, 2 or 3 substituents selected from the group consisting of methyl, —Cl, —F, —CN, —O—(CH₂)₂-piperidinyl, —O—(CH₂)₂-morpholinyl, —O—(CH₂)₃—N(CH₃)₂, and —CH₂-morpholinyl;
B is a ring system selected from the group consisting of 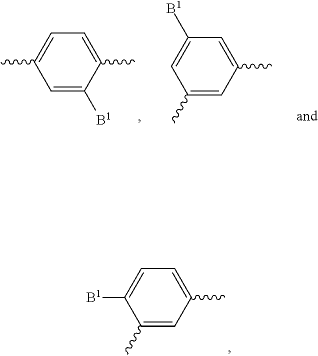
wherein B¹ is —SCH₃,
C is selected from the group consisting of 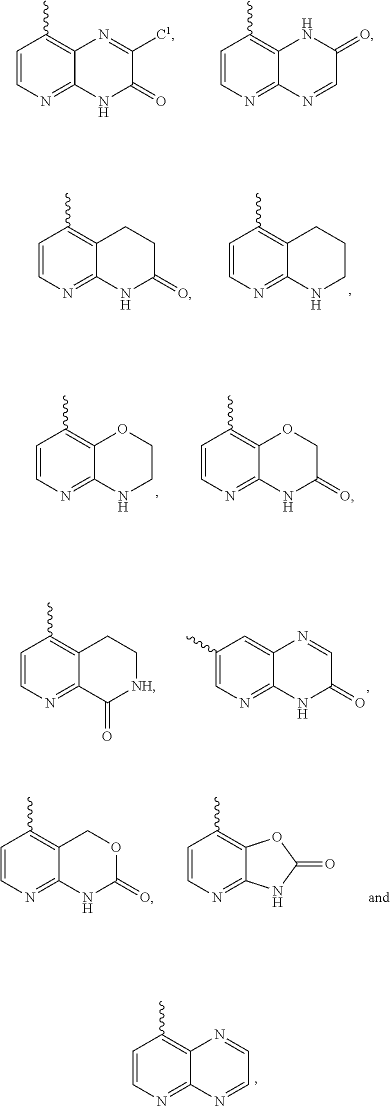
wherein C¹ is selected from the group consisting of —H, —CH₃, —OCH₃, —CN or 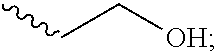
or
wherein A is 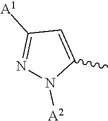
A¹ is tert-butyl,
and A² is selected from the group consisting of a phenyl substituted with 1, 2 or 3 substituents selected from the group consisting of —Cl, —F, —CN, —O—(CH₂)₂-piperidinyl, —O—(CH₂)₃—N(CH₃)₂, and —CH₂-morpholinyl; a pyridinyl substituted with a methyl; and a phenyl substituted by two methyls;
B is 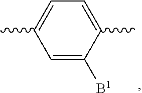
with B¹ being —SCH₃,
C is selected in the group consisting of

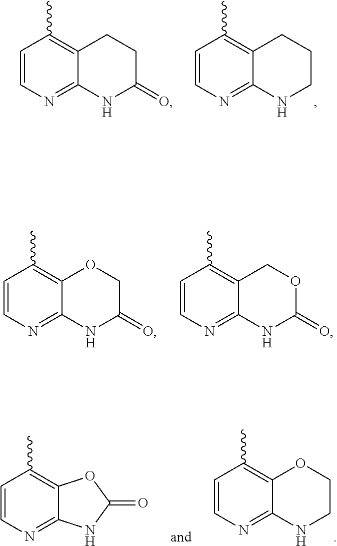

68. The method according to claim 60, wherein the compound is selected in the group consisting of
1-(3-(tert-butyl)-1-(4-methoxyphenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(3-chlorophenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(3-methoxyphenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(3-fluorophenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(quinolin-6-yl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(4-cyanophenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(3,4-dimethylphenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(4-(2-(piperidin-1-yl)ethoxy)phenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(4-(2-morpholinoethoxy)phenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(3-chloro-4-methylphenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(3-chloro-4-fluorophenyl)-1H-pyrazol-5-yl)-3-(2-(methylthio)-4-((3-oxo-3,4-dihydropyrido[2,3-b]pyrazin-8-yl)oxy)phenyl)urea;
1-(3-(tert-butyl)-1-(4-(3-(dimethylamino)propoxy)phenyl)-1H-pyrazol-5-yl)-3-(2-(methylthio)-4-((3-oxo-3,4-dihydropyrido[2,3-b]pyrazin-8-yl)oxy)phenyl)urea; and
1-(3-(tert-butyl)-1-(4-(morpholinomethyl)phenyl)-1H-pyrazol-5-yl)-3-(2-(methylthio)-4-((3-oxo-3,4-dihydropyrido[2,3-b]pyrazin-8-yl)oxy)phenyl)urea;
or a pharmaceutically acceptable salt thereof.

69. The method according to claim 60,
wherein A is 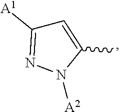
A¹ is selected from the group consisting of methyl, —CF₃, isopropyl, cyclopropyl, isobutyl, sec-butyl, tert-butyl, 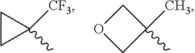
A² is 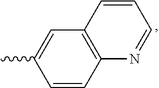
B is a ring system selected from the group consisting of 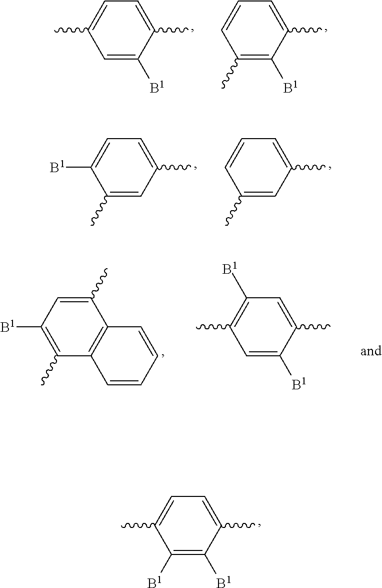
wherein, when B is a phenyl, B¹ is selected from the group consisting of —F, —Cl, —SCH₃, —SCH₂CH₃, —CH₃, —CF₃, —OCH₃, and —OCF₃ and, when B is a naphtyl, B¹ is —H, and
C is selected from the group consisting of 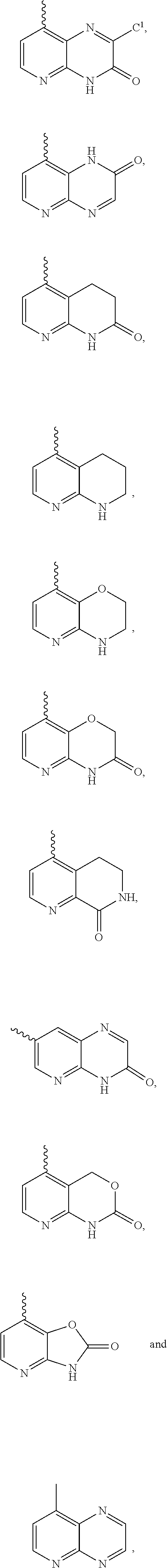
wherein C¹ is selected from the group consisting of —H, —CH₃, —OCH₃, —CN or 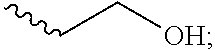
or
wherein A is 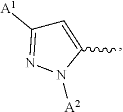
A¹ is tert-butyl,
A² is 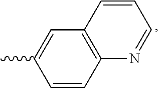
B is a ring system selected from the group consisting of 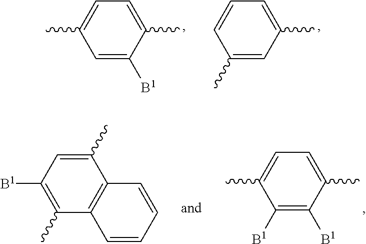
wherein, when B is a phenyl, B¹ is selected from the group consisting of —F, —Cl, —SCH₃, —SCH₂CH₃, —CH₃, —CF₃, —OCH₃, and —OCF₃ and, when B is a naphtyl, B¹ is —H, and
C is selected from the group consisting of

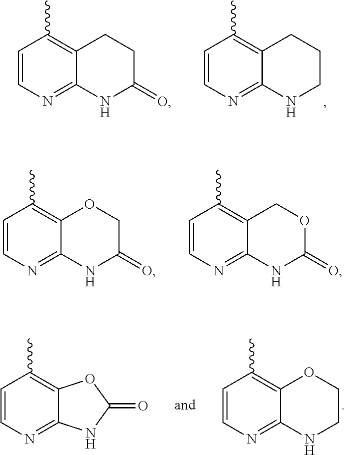

70. The method according to claim 60, wherein the compound is selected in the group consisting of
1-(3-(tert-butyl)-1-(quinolin-6-yl)-1H-pyrazol-5-yl)-3-(2-fluoro-4-((3-oxo-3,4-dihydropyrido[2,3-b]pyrazin-8-yl)oxy)phenyl)urea;
1-(3-(tert-butyl)-1-(quinolin-6-yl)-1H-pyrazol-5-yl)-3-(4-((3-oxo-3,4-dihydropyrido[2,3-b]pyrazin-8-yl)oxy)naphthalen-1-yl)urea; and
1-(3-(tert-butyl)-1-(quinolin-6-yl)-1H-pyrazol-5-yl)-3-(2-(methylthio)-4-((3-oxo-3,4-dihydropyrido[2,3-b]pyrazin-8-yl)oxy)phenyl)urea;
or a pharmaceutically acceptable salt thereof.

71. The method according to claim 60,
wherein A is a ring selected from the group consisting of 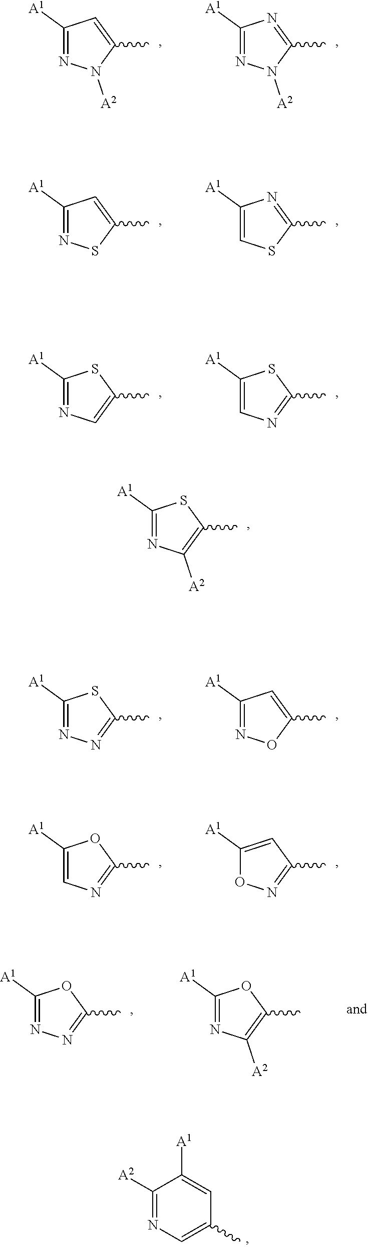
A¹ is selected from the group consisting of methyl, —CF₃, isopropyl, cyclopropyl, isobutyl, sec-butyl, tert-butyl, 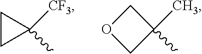
and A² is selected from the group consisting of H, methyl, iso-propyl, benzyl, phenyl, chlorine, 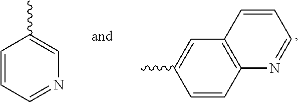
wherein A¹ and A² are optionally fluorinated, and A₂ is optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,
B is a ring system selected from the group consisting of 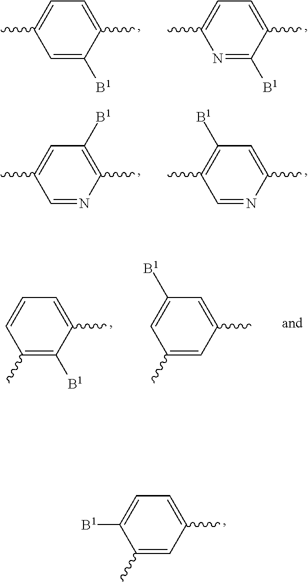
wherein B¹ is —SCH₃,
C is selected from the group consisting of 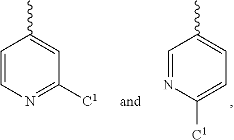
with C¹ being selected C¹ is selected from the group consisting of wherein C¹ is selected from the group consisting of —H, —NH₂, —NR¹, —C(O)—NH—R¹, —NH—C(O)—R¹, —NH—S(O)₂—R¹, —NH—C(O)—OR¹, —C(O)—R¹, —R¹, —OR¹, —SO₂R¹, with R¹ being selected from the group consisting of C₁-C₁₀ alkyl, C₆-C₁₄ aryl, C₃-C₁₃ heteroaryl, and C₇-C₂₄ alkaryl, wherein each hydrogen in each of these groups may be independently replaced by a group selected from a halogen, a hydroxy, —OR′, and —NR′R″, each hydrogen in each of R′ and R″ may be independently replaced by —COR′″ or COOR′″, wherein R′, R″ and R′″ are independently a C₁-C₆ alkyl;
or
wherein A is a ring selected from the group consisting of 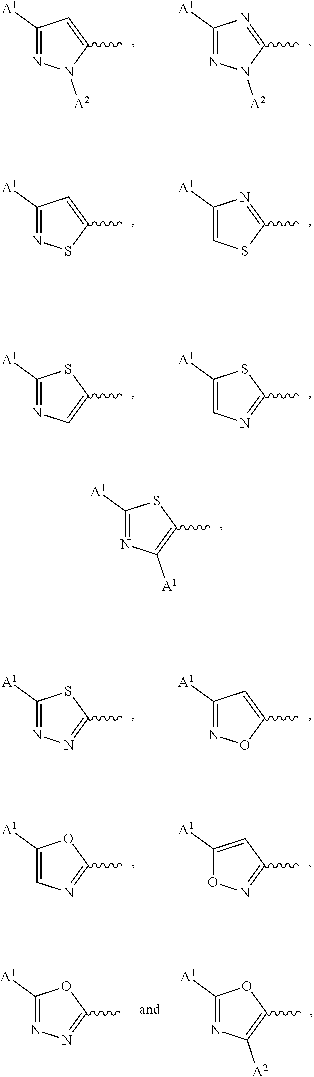
A¹ is tert-butyl,
A² is a phenyl or 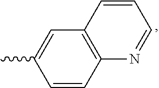
optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,
B is a ring system selected from the group consisting of 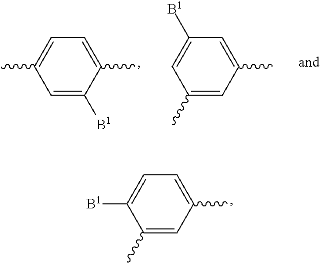
wherein B¹ is —SCH₃,
C is selected from the group consisting of 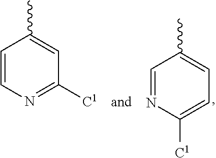
with C¹ being selected from the group consisting of —H, —NH₂, 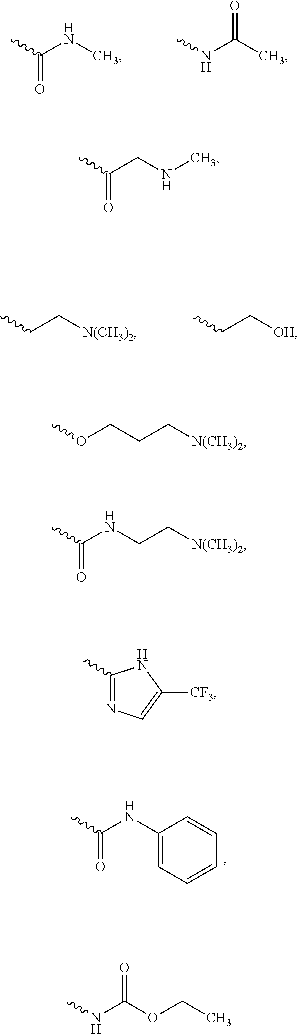
—NH—C(O)OtBu, —NH—C(O)N(CH3)-C(O)OtBu, —NH—C(O)—CH₂—N(CH₃)—C(O)OtBu, —NH—C(O)—CH₂—NHCH₃, —NH—C(O)-Obenzyl, —NH—C(O)—CH₂—OH, —NH—(CH₂)₂—N(CH₃)₂, and —NH—S(O)₂—CH₃, preferably from the group consisting of —H, 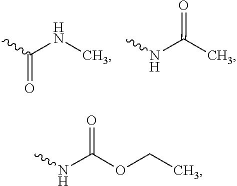
—NH—C(O)OtBu, —NH—C(O)N(CH₃)—C(O)OtBu, —NH—C(O)—CH₂—N(CH₃)—C(O)OtBu, —NH—C(O)—CH₂—NHCH₃, —NH—C(O)—Obenzyl, and —NH—C(O)—CH₂—OH;
or
wherein A is a ring selected from the group consisting of 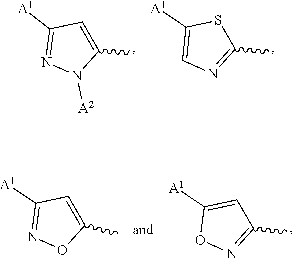
A¹ is tert-butyl,
A² is a phenyl optionally substituted with 1, 2 or 3 substituents selected from the group consisting of methyl, —Cl, —F, —CN, methoxy, —O—(CH₂)₂-piperidinyl, —O—(CH₂)₂-morpholinyl, —O—(CH₂)₃—N(CH₃)₂, and —CH₂-morpholinyl, or A² is 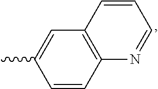
B is 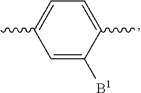
with B¹ being —SCH₃,
C is selected from the group consisting of 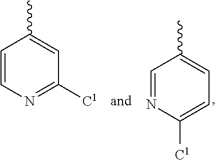
with C¹ being selected from the group consisting of 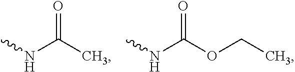
—NH—C(O)OtBu, —NH—C(O)—CH₂—N(CH₃)—C(O)OtBu and —NH—C(O)N(CH₃)—COOtBu.

72. The method according to claim 60, wherein the compound is selected in the group consisting of
5-(4-(3-(3-(tert-butyl)-1-phenyl-1H-pyrazol-5-yl)ureido)-3-(methylthio)phenoxy)-N-methylnicotinamide;
N-(4-(4-(3-(3-(tert-butyl)-1-phenyl-1H-pyrazol-5-yl)ureido)-3-(methylthio)phenoxy)pyridin-2-yl)acetamide;
tert-butyl (4-(4-(3-(3-(tert-butyl)-1-phenyl-1H-pyrazol-5-yl)ureido)-3-(methylthio)phenoxy)pyridin-2-yl)carbamate;
tert-butyl (2-((4-(4-(3-(3-(tert-butyl)-1-phenyl-1H-pyrazol-5-yl)ureido)-3-(methylthio)phenoxy)pyridin-2-yl)amino)-2-oxoethyl)(methyl)carbamate;
N-(4-(4-(3-(3-(tert-butyl)-1-phenyl-1H-pyrazol-5-yl)ureido)-3-(methylthio)phenoxy)pyridin-2-yl)-2-(methylamino)acetamide;
1-(3-(tert-butyl)-1-phenyl-1H-pyrazol-5-yl)-3-(4-((2-((2-(dimethylamino)ethyl)amino)pyridin-4-yl)oxy)-2-(methylthio)phenyl)urea hydrochloride;
ethyl (4-(4-(3-(3-(tert-butyl)-1-phenyl-1H-pyrazol-5-yl)ureido)-3-(methylthio)phenoxy)pyridin-2-yl)carbamate;
benzyl (4-(4-(3-(3-(tert-butyl)-1-phenyl-1H-pyrazol-5-yl)ureido)-3-(methylthio)phenoxy)pyridin-2-yl)carbamate;
ethyl (4-(4-(3-(3-(tert-butyl)isoxazol-5-yl)ureido)-3-(methylthio)phenoxy)pyridin-2-yl)carbamate;
ethyl (4-(4-(3-(5-(tert-butyl)thiazol-2-yl)ureido)-3-(methylthio)phenoxy)pyridin-2-yl)carbamate; and
ethyl (4-(4-(3-(5-(tert-butyl)isoxazol-3-yl)ureido)-3-(methylthio)phenoxy)pyridin-2-yl)carbamate;
or a pharmaceutically acceptable salt thereof.

73. The method according to claim 60,
wherein A is a ring system selected from the group consisting of 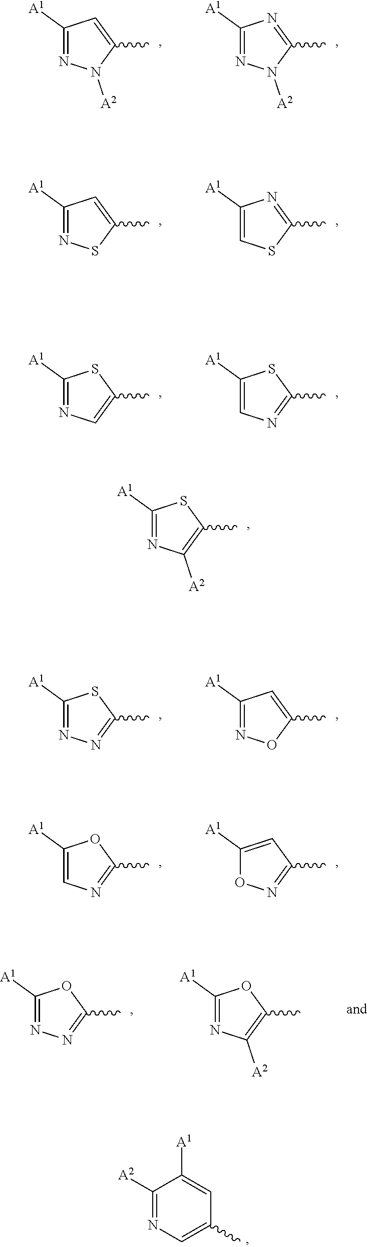
A¹ is selected from the group consisting of methyl, —CF₃, isopropyl, cyclopropyl, isobutyl, sec-butyl, tert-butyl, 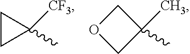
and A² is selected from the group consisting of H, methyl, iso-propyl, benzyl, phenyl, chlorine, 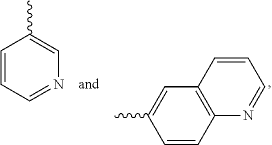
wherein A¹ and A² are optionally fluorinated, and A² is optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,
B is a ring system selected from the group consisting of 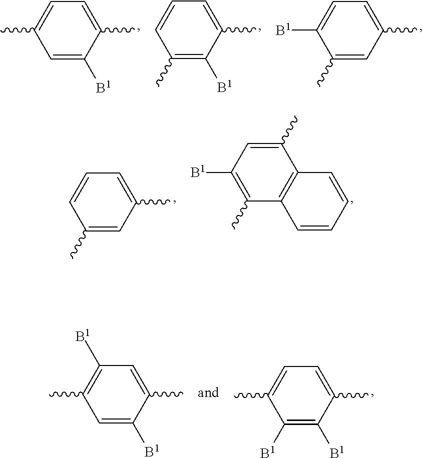
wherein, when B is a phenyl, B¹ is selected from the group consisting of —F, —Cl, —SCH₃, —SCH₂CH₃, —CH₃, —CF₃, —OCH₃, and —OCF₃ and, when B is a naphtyl, B¹ is —H, and
C is selected from the group consisting of 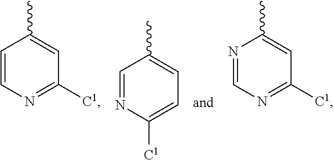
wherein C¹ is selected from the group consisting of —NH—C(O)R¹ or —NH—C(O)—OR¹ with R¹ being selected from the group consisting of C₁-C₁₀ alkyl, C₆-C₁₄ aryl, C₃-C₁₃ heteroaryl, and C₇-C₂₄ alkaryl, wherein each hydrogen in each of these groups may be independently replaced by a group selected from halogen, hydroxy, —OR′, and —NR′R″, each hydrogen in each of R′ and R″ may be independently replaced by —COR′ or COOR′″, wherein R′, R″ and R′″ are independently a C₁-C₆ alkyl, preferably selected from the group consisting of —NH—C(O)R¹ or —NH—C(O)—OR¹ with R¹ being selected from the group consisting of C₁-C₁₀ alkyl, C₆-C₁₄ aryl, C₃-C₁₃ heteroaryl, and C₇-C₂₄ alkaryl, wherein each hydrogen in each of these groups may be independently replaced by a group selected from halogen, hydroxy, and —NR′R″, each hydrogen in each of R′ and R″ may be independently replaced by —COR′″ or COOR′″, wherein R′, R″ and R′″ are independently a C₁-C₆ alkyl;
or
wherein A is a ring selected from the group consisting of 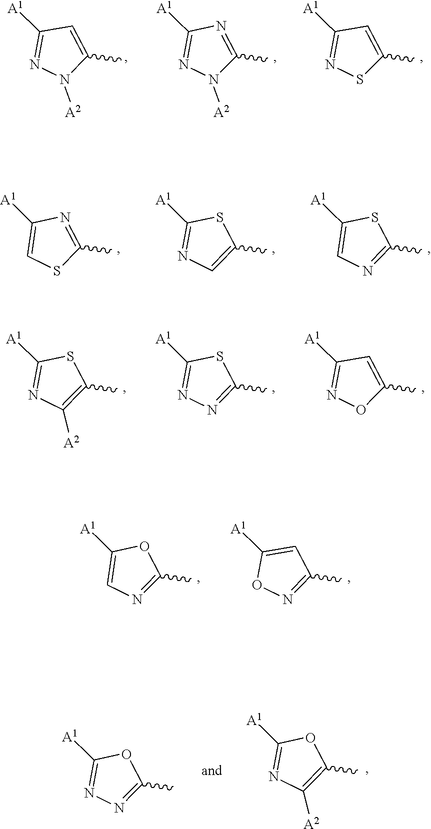
A¹ is tert-butyl,
A² is a phenyl or 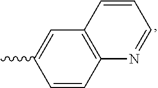
optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,
B is a ring system selected from the group consisting of 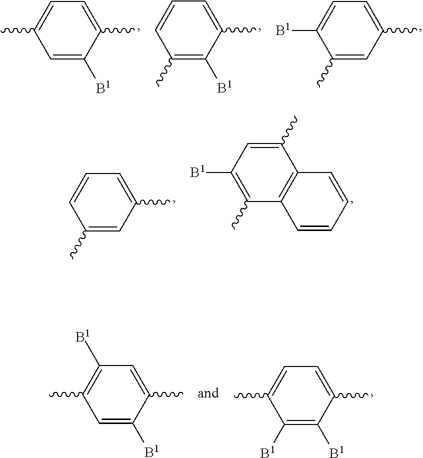
wherein, when B is a phenyl, B¹ is selected from the group consisting of —F, —SCH₃, —SCH₂CH₃, —CF₃, —OCH₃, and —OCF₃ and, when B is a naphtyl, B¹ is —H, and
C is selected from the group consisting of 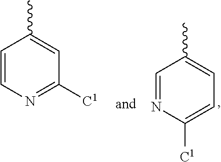
with C¹ being selected from the group consisting of 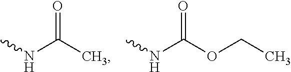
—NH—C(O)OtBu, —NH—C(O)N(CH3)-C(O)OtBu, —NH—C(O)—CH₂—N(CH₃)—C(O)OtBu, —NH—C(O)—CH₂—NHCH₃, —NH—C(O)—Obenzyl, —NH—C(O)—CH₂—OH, preferably 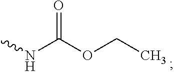
or
wherein A is a ring selected from the group consisting of 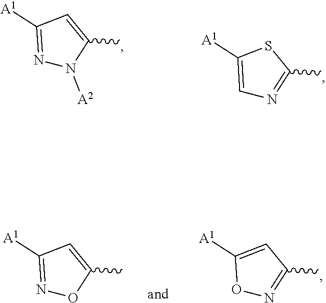
A¹ is tert-butyl,
A² is a phenyl optionally substituted with 1, 2 or 3 substituents selected from the group consisting of methyl, —Cl, —F, —CN, methoxy, —O—(CH₂)₂-piperidinyl, —O—(CH₂)₂-morpholinyl, —O—(CH₂)₃—N(CH₃)₂, and —CH₂-morpholinyl, or A² is 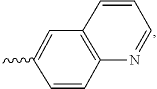
B is a ring system selected in the group consisting of 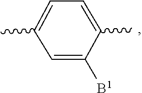
with B¹ being selected from the group consisting of —F, —SCH₃, —SCH₂CH₃, —CF₃, —OCH₃, and —OCF₃, preferably —SCH₃, 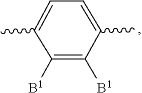
with B¹ being F,
and 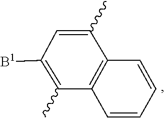
with B¹ being —H, and
C is selected in the group consisting of 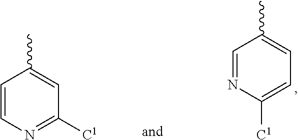
with C¹ being selected from the group consisting of 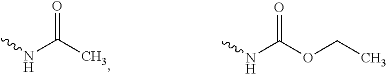
—NH—C(O)OtBu, —NH—C(O)N(CH3)-C(O)OtBu, —NH—C(O)—CH₂—N(CH₃)—C(O)OtBu, —NH—C(O)—CH₂—NHCH₃, —NH—C(O)—Obenzyl, —NH—C(O)—CH₂—OH, preferably

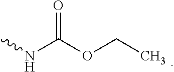

74. The method according to claim 60, wherein the compound is selected in the group consisting of
N-(4-(4-(3-(3-(tert-butyl)-1-phenyl-1H-pyrazol-5-yl)ureido)-3-fluorophenoxy)pyridin-2-yl)acetamide;
N-(4-(4-(3-(3-(tert-butyl)-1-phenyl-1H-pyrazol-5-yl)ureido)-3-(methylthio)phenoxy)pyridin-2-yl)acetamide;
tert-butyl (4-(4-(3-(3-(tert-butyl)-1-phenyl-1H-pyrazol-5-yl)ureido)-3-(methylthio)phenoxy)pyridin-2-yl)carbamate;
tert-butyl (2-((4-(4-(3-(3-(tert-butyl)-1-phenyl-1H-pyrazol-5-yl)ureido)-3-(methylthio)phenoxy)pyridin-2-yl)amino)-2-oxoethyl)(methyl)carbamate;
ethyl (4-(4-(3-(3-(tert-butyl)-1-phenyl-1H-pyrazol-5-yl)ureido)-3-(methylthio)phenoxy)pyridin-2-yl)carbamate
ethyl (4-((4-(3-(3-(tert-butyl)-1-phenyl-1H-pyrazol-5-yl)ureido)naphthalen-1-yl)oxy)pyridin-2-yl)carbamate;
ethyl (4-((4-(3-(3-(tert-butyl)isoxazol-5-yl)ureido)naphthalen-1-yl)oxy)pyridin-2-yl)carbamate;
ethyl (4-((4-(3-(5-(tert-butyl)thiazol-2-yl)ureido)naphthalen-1-yl)oxy)pyridin-2-yl)carbamate;
ethyl (4-(4-(3-(3-(tert-butyl)isoxazol-5-yl)ureido)-3-(methylthio)phenoxy)pyridin-2-yl)carbamate;
ethyl (4-(4-(3-(5-(tert-butyl)thiazol-2-yl)ureido)-3-(methylthio)phenoxy)pyridin-2-yl)carbamate;
benzyl (4-(4-(3-(3-(tert-butyl)-1-phenyl-1H-pyrazol-5-yl)ureido)-3-(methylthio)phenoxy)pyridin-2-yl)carbamate;
N-(4-(4-(3-(3-(tert-butyl)-1-phenyl-1H-pyrazol-5-yl)ureido)-3-(methylthio)phenoxy)pyridin-2-yl)-2-hydroxyacetamide; and
tert-butyl (4-(4-(3-(3-(tert-butyl)-1-phenyl-1H-pyrazol-5-yl)ureido)-3-(methylthio)phenoxy)pyridin-2-yl)(2-(dimethylamino)ethyl)carbamate;
or a pharmaceutically acceptable salt thereof.

75. The method according to claim 60, wherein said disease or condition being selected from the group consisting of Duchenne muscular dystrophy, Becker muscular dystrophy, cachexia and sarcopenia, preferably selected from the group consisting of Duchenne muscular dystrophy, Becker muscular dystrophy, and sarcopenia, more preferably Duchenne muscular dystrophy or Becker muscular dystrophy.

76. A compound selected from the group consisting of

| Compound | Structure | Name |
|---|---|---|
| (a) | 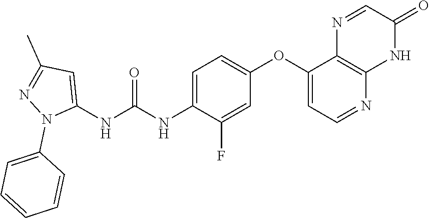 [SMILES: Cc1cc(NC(=O)Nc2ccc(Oc3ccnc4[nH]c(=O)cnc34)cc2F)n(-c2ccccc2)n1] | 1-(2-fluoro-4-((3-oxo-3,4- dihydropyrido[2,3-b]pyrazin-8- yl)oxy)phenyl)-3-(3-methyl-1- phenyl-1H-pyrazol-5-yl)urea |
| (b) | 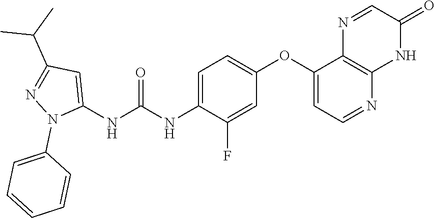 [SMILES: CC(C)c1cc(NC(=O)Nc2ccc(Oc3ccnc4[nH]c(=O)cnc34)cc2F)n(-c2ccccc2)n1] | 1-(2-fluoro-4-((3-oxo-3,4- dihydropyrido[2,3-b]pyrazin-8- yl)oxy)phenyl)-3-(3-isopropyl-1- phenyl-1H-pyrazol-5-yl)urea |
| (c) | 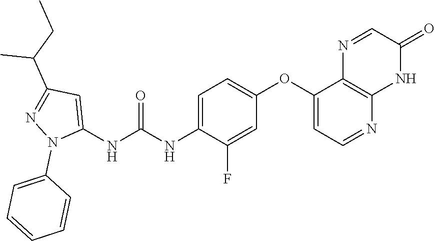 [SMILES: CCC(C)c1cc(NC(=O)Nc2ccc(Oc3ccnc4[nH]c(=O)cnc34)cc2F)n(-c2ccccc2)n1] | 1-(3-(sec-butyl)-1-phenyl-1H- pyrazol-5-yl)-3-(2-fluoro-4-((3-oxo- 3,4-dihydropyrido[2,3-b]pyrazin-8- yl)oxy)phenyl)urea |
| (d) | 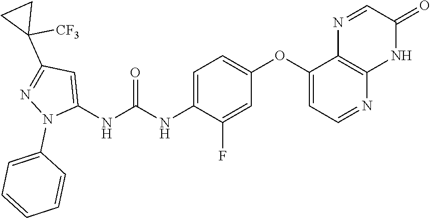 [SMILES: O=C(Nc1ccc(Oc2ccnc3[nH]c(=O)cnc23)cc1F)Nc1cc(C2(C(F)(F)F)CC2)nn1-c1ccccc1] | 1-(2-fluoro-4-((3-oxo-3,4- dihydropyrido[2,3-b]pyrazin-8- yl)oxy)phenyl)-3-(1-phenyl-3-(1- (trifluoromethyl)cyclopropyl)-1H- pyrazol-5-yl)urea |
| (e) | 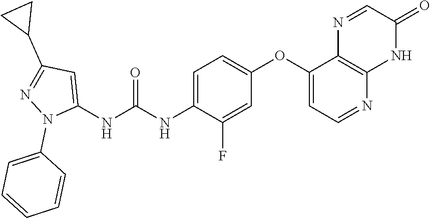 [SMILES: O=C(Nc1ccc(Oc2ccnc3[nH]c(=O)cnc23)cc1F)Nc1cc(C2CC2)nn1-c1ccccc1] | 1-(3-cyclopropyl-1-phenyl-1H- pyrazol-5-yl)-3-(2-fluoro-4-((3-oxo- 3,4-dihydropyrido[2,3-b]pyrazin-8- yl)oxy)phenyl)urea |
| (f) | 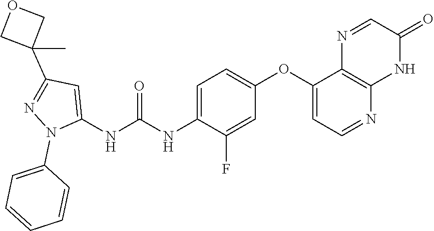 [SMILES: CC1(c2cc(NC(=O)Nc3ccc(Oc4ccnc5[nH]c(=O)cnc45)cc3F)n(-c3ccccc3)n2)COC1] | 1-(2-fluoro-4-((3-oxo-3,4- dihydropyrido[2,3-b]pyrazin-8- yl)oxy)phenyl)-3-(3-(3- methyloxetan-3-yl)-1-phenyl-1H- pyrazol-5-yl)urea |
| (g) | 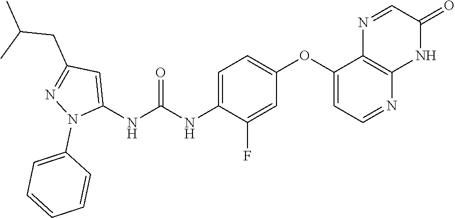 [SMILES: CC(C)Cc1cc(NC(=O)Nc2ccc(Oc3ccnc4[nH]c(=O)cnc34)cc2F)n(-c2ccccc2)n1] | 1-(2-fluoro-4-((3-oxo-3,4- dihydropyrido[2,3-b]pyrazin-8- yl)oxy)phenyl)-3-(3-isobutyl-1- phenyl-1H-pyrazol-5-yl)urea |
| (h) | 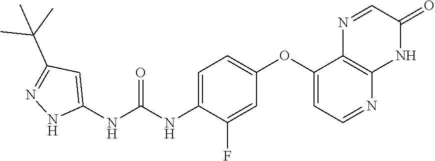 [SMILES: CC(C)(C)c1cc(NC(=O)Nc2ccc(Oc3ccnc4[nH]c(=O)cnc34)cc2F)[nH]n1] | 1-(3-(tert-butyl)-1H-pyrazol-5-yl)-3- (2-fluoro-4-((3-oxo-3,4- dihydropyrido[2,3-b]pyrazin-8- yl)oxy)phenyl)urea |
| (i) | 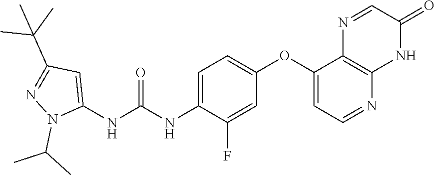 [SMILES: CC(C)n1nc(C(C)(C)C)cc1NC(=O)Nc1ccc(Oc2ccnc3[nH]c(=O)cnc23)cc1F] | 1-(3-(tert-butyl)-1-isopropyl-1H- pyrazol-5-yl)-3-(2-fluoro-4-((3-oxo- 3,4-dihydropyrido[2,3-b]pyrazin-8- yl)oxy)phenyl)urea |
| (j) | 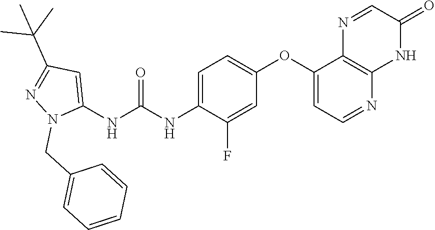 [SMILES: CC(C)(C)c1cc(NC(=O)Nc2ccc(Oc3ccnc4[nH]c(=O)cnc34)cc2F)n(Cc2ccccc2)n1] | 1-(1-benzyl-3-(tert-butyl)-1H- pyrazol-5-yl)-3-(2-fluoro-4-((3-oxo- 3,4-dihydropyrido[2,3-b]pyrazin-8- yl)oxy)phenyl)urea |
| (k) | 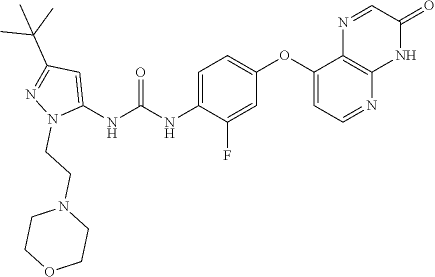 [SMILES: CC(C)(C)c1cc(NC(=O)Nc2ccc(Oc3ccnc4[nH]c(=O)cnc34)cc2F)n(CCN2CCOCC2)n1] | 1-(3-(tert-butyl)-1-(2- morpholinoethyl)-1H-pyrazol-5-yl)- 3-(2-fluoro-4-((3-oxo-3,4- dihydropyrido[2,3-b]pyrazin-8- yl)oxy)phenyl)urea |
| (l) | 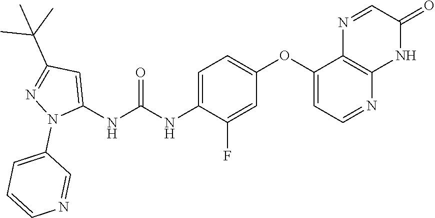 [SMILES: CC(C)(C)c1cc(NC(=O)Nc2ccc(Oc3ccnc4[nH]c(=O)cnc34)cc2F)n(-c2cccnc2)n1] | 1-(3-(tert-butyl)-1-(pyridin-3-yl)-1H- pyrazol-5-yl)-3-(2-fluoro-4-((3-oxo- 3,4-dihydropyrido[2,3-b]pyrazin-8- yl)oxy)phenyl)urea |
| (m) | 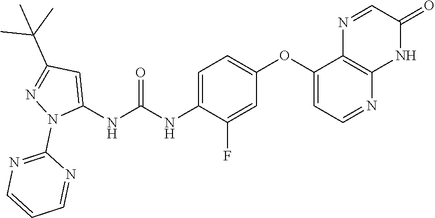 [SMILES: CC(C)(C)c1cc(NC(=O)Nc2ccc(Oc3ccnc4[nH]c(=O)cnc34)cc2F)n(-c2ncccn2)n1] | 1-(3-(tert-butyl)-1-(pyrimidin-2-yl)- 1H-pyrazol-5-yl)-3-(2-fluoro-4-((3- oxo-3,4-dihydropyrido[2,3- b]pyrazin-8-yl)oxy)phenyl)urea |
| (n) | 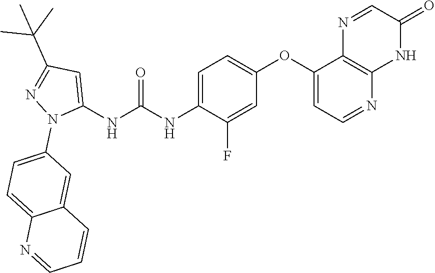 [SMILES: CC(C)(C)c1cc(NC(=O)Nc2ccc(Oc3ccnc4[nH]c(=O)cnc34)cc2F)n(-c2ccc3ncccc3c2)n1] | 1-(3-(tert-butyl)-1-(quinolin-6-yl)- 1H-pyrazol-5-yl)-3-(2-fluoro-4-((3- oxo-3,4-dihydropyrido[2,3- b]pyrazin-8-yl)oxy)phenyl)urea |
| (o) | 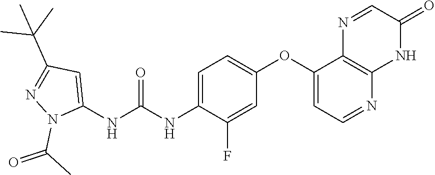 [SMILES: CC(=O)n1nc(C(C)(C)C)cc1NC(=O)Nc1ccc(Oc2ccnc3[nH]c(=O)cnc23)cc1F] | 1-(1-acetyl-3-(tert-butyl)-1H- pyrazol-5-yl)-3-(2-fluoro-4-((3-oxo- 3,4-dihydropyrido[2,3-b]pyrazin-8- yl)oxy)phenyl)urea |
| (p) | 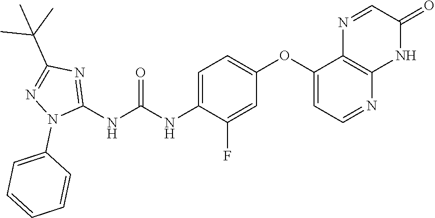 [SMILES: CC(C)(C)c1nc(NC(=O)Nc2ccc(Oc3ccnc4[nH]c(=O)cnc34)cc2F)n(-c2ccccc2)n1] | 1-(3-(tert-butyl)-1-phenyl-1H-1,2,4- triazol-5-yl)-3-(2-fluoro-4-((3-oxo- 3,4-dihydropyrido[2,3-b]pyrazin-8- yl)oxy)phenyl)urea |
| (q) | 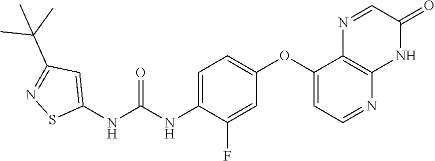 [SMILES: CC(C)(C)c1cc(NC(=O)Nc2ccc(Oc3ccnc4[nH]c(=O)cnc34)cc2F)sn1] | 1-(3-(tert-butyl)isothiazol-5-yl)-3-(2- fluoro-4-((3-oxo-3,4- dihydropyrido[2,3-b]pyrazin-8- yl)oxy)phenyl)urea |
| (r) | 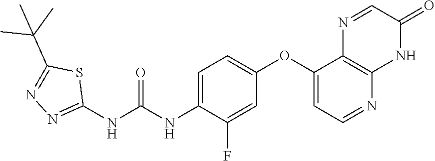 [SMILES: CC(C)(C)c1nnc(NC(=O)Nc2ccc(Oc3ccnc4[nH]c(=O)cnc34)cc2F)s1] | 1-(5-(tert-butyl)-1,3,4-thiadiazol-2- yl)-3-(2-fluoro-4-((3-oxo-3,4- dihydropyrido[2,3-b]pyrazin-8- yl)oxy)phenyl)urea |
| (s) | 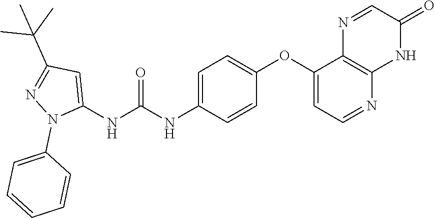 [SMILES: CC(C)(C)c1cc(NC(=O)Nc2ccc(Oc3ccnc4[nH]c(=O)cnc34)cc2)n(-c2ccccc2)n1] | 1-(3-(tert-butyl)-1-phenyl-1H- pyrazol-5-yl)-3-(4-((3-oxo-3,4- dihydropyrido[2,3-b]pyrazin-8- yl)oxy)phenyl)urea |
| (t) | 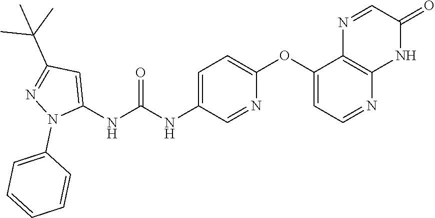 [SMILES: CC(C)(C)c1cc(NC(=O)Nc2ccc(Oc3ccnc4[nH]c(=O)cnc34)nc2)n(-c2ccccc2)n1] | 1-(3-(tert-butyl)-1-phenyl-1H- pyrazol-5-yl)-3-(6-((3-oxo-3,4- dihydropyrido[2,3-b]pyrazin-8- yl)oxy)pyridin-3-yl)urea |
| (u) | 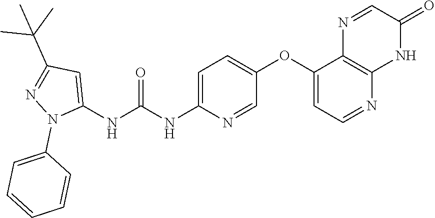 [SMILES: CC(C)(C)c1cc(NC(=O)Nc2ccc(Oc3ccnc4[nH]c(=O)cnc34)cn2)n(-c2ccccc2)n1] | 1-(3-(tert-butyl)-1-phenyl-1H- pyrazol-5-yl)-3-(5-((3-oxo-3,4- dihydropyrido[2,3-b]pyrazin-8- yl)oxy)pyridin-2-yl)urea |
| (v) | 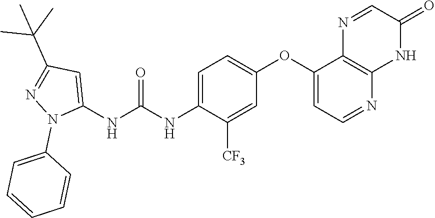 [SMILES: CC(C)(C)c1cc(NC(=O)Nc2ccc(Oc3ccnc4[nH]c(=O)cnc34)cc2C(F)(F)F)n(-c2ccccc2)n1] | 1-(3-(tert-butyl)-1-phenyl-1H- pyrazol-5-yl)-3-(4-((3-oxo-3,4- dihydropyrido[2,3-b]pyrazin-8- yl)oxy)-2- (trifluoromethyl)phenyl)urea |
| (w) | 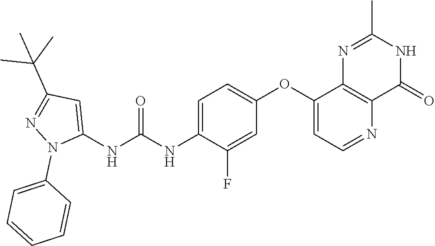 [SMILES: Cc1nc2c(Oc3ccc(NC(=O)Nc4cc(C(C)(C)C)nn4-c4ccccc4)c(F)c3)ccnc2c(=O)[nH]1] | 1-(3-(tert-butyl)-1-phenyl-1H- pyrazol-5-yl)-3-(2-fluoro-4-((2- methyl-4-oxo-3,4-dihydropyrido[3,2- d]pyrimidin-8-yl)oxy)phenyl)urea |
| (x) | 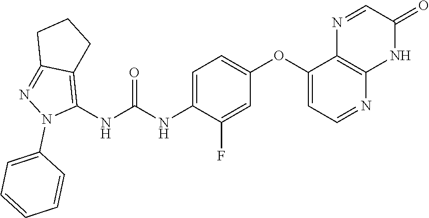 [SMILES: O=C(Nc1ccc(Oc2ccnc3[nH]c(=O)cnc23)cc1F)Nc1c2c(nn1-c1ccccc1)CCC2] | 1-(2-fluoro-4-((3-oxo-3,4- dihydropyrido[2,3-b]pyrazin-8- yl)oxy)phenyl)-3-(2-phenyl-2,4,5,6- tetrahydrocyclopenta[c]pyrazol-3- yl)urea |
| (y) | 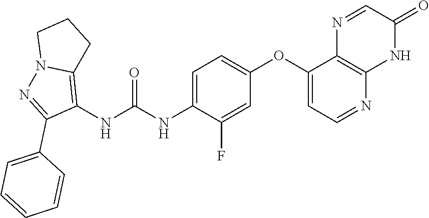 [SMILES: O=C(Nc1ccc(Oc2ccnc3[nH]c(=O)cnc23)cc1F)Nc1c(-c2ccccc2)nn2c1CCC2] | 1-(2-fluoro-4-((3-oxo-3,4- dihydropyrido[2,3-b]pyrazin-8- yl)oxy)phenyl)-3-(2-phenyl-5,6- dihydro-4H-pyrrolo[1,2-b]pyrazol-3- yl)urea |
| (z) | 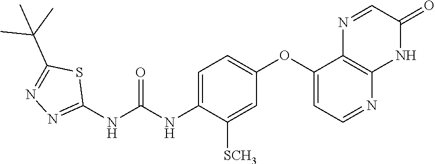 [SMILES: CSc1cc(Oc2ccnc3[nH]c(=O)cnc23)ccc1NC(=O)Nc1nnc(C(C)(C)C)s1] | 1-(5-(tert-butyl)-1,3,4-thiadiazol-2- yl)-3-(2-(methylthio)-4-((3-oxo-3,4- dihydropyrido[2,3-b]pyrazin-8- yl)oxy)phenyl)urea |
| (aa) | 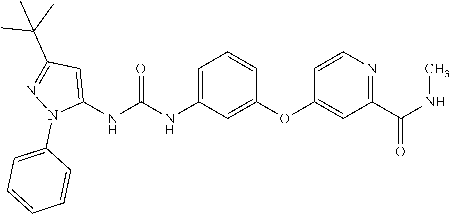 [SMILES: CNC(=O)c1cc(Oc2cccc(NC(=O)Nc3cc(C(C)(C)C)nn3-c3ccccc3)c2)ccn1] | 4-(3-(3-(3-(tert-butyl)-1-phenyl-1H- pyrazol-5-yl)ureido)phenoxy)-N- methylpicolinamide |
| (ab) | 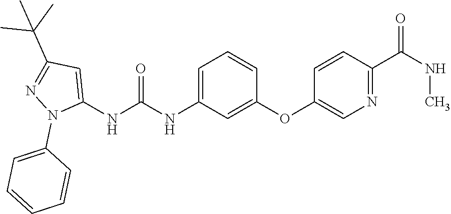 [SMILES: CNC(=O)c1ccc(Oc2cccc(NC(=O)Nc3cc(C(C)(C)C)nn3-c3ccccc3)c2)cn1] | 5-(3-(3-(3-(tert-butyl)-1-phenyl-1H- pyrazol-5-yl)ureido)phenoxy)-N- methylpicolinamide |
| (ac) | 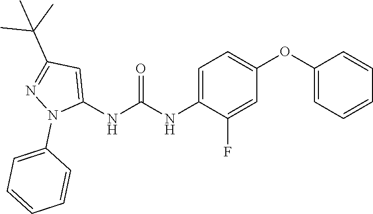 [SMILES: CC(C)(C)c1cc(NC(=O)Nc2ccc(Oc3ccccc3)cc2F)n(-c2ccccc2)n1] | 1-(3-(tert-butyl)-1-phenyl-1H- pyrazol-5-yl)-3-(2-fluoro-4- phenoxyphenyl)urea |
| (ad) | 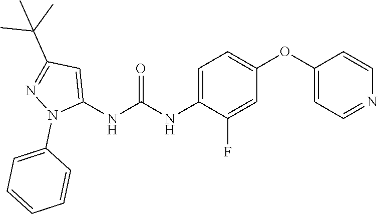 [SMILES: CC(C)(C)c1cc(NC(=O)Nc2ccc(Oc3ccncc3)cc2F)n(-c2ccccc2)n1] | 1-(3-(tert-butyl)-1-phenyl-1H- pyrazol-5-yl)-3-(2-fluoro-4-(pyridin- 4-yloxy)phenyl)urea |
| (ae) | 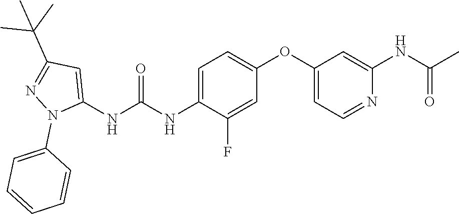 [SMILES: CC(=O)Nc1cc(Oc2ccc(NC(=O)Nc3cc(C(C)(C)C)nn3-c3ccccc3)c(F)c2)ccn1] | N-(4-(4-(3-(3-(tert-butyl)-1-phenyl- 1H-pyrazol-5-yl)ureido)-3- fluorophenoxy)pyridin-2- yl)acetamide |
| (af) | 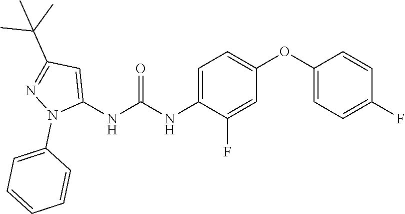 [SMILES: CC(C)(C)c1cc(NC(=O)Nc2ccc(Oc3ccc(F)cc3)cc2F)n(-c2ccccc2)n1] | 1-(3-(tert-butyl)-1-phenyl-1H- pyrazol-5-yl)-3-(2-fluoro-4-(4- fluorophenoxy)phenyl)urea |
| (ag) | 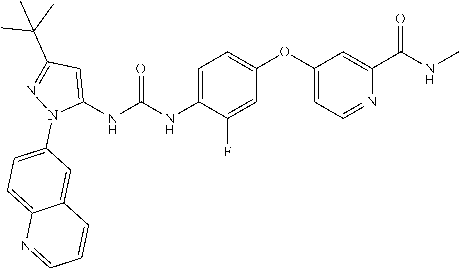 [SMILES: CNC(=O)c1cc(Oc2ccc(NC(=O)Nc3cc(C(C)(C)C)nn3-c3ccc4ncccc4c3)c(F)c2)ccn1] | 4-(4-(3-(3-(tert-butyl)-1-(quinolin-6- yl)-1H-pyrazol-5-yl)ureido)-3- (methylthio)phenoxy)-N- methylpicolinamide |
| (ah) | 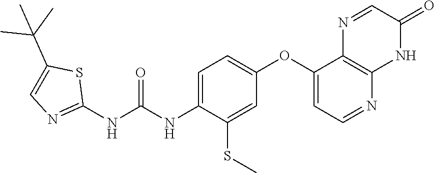 [SMILES: CSc1cc(Oc2ccnc3[nH]c(=O)cnc23)ccc1NC(=O)Nc1ncc(C(C)(C)C)s1] | 1-(5-(tert-butyl)thiazol-2-yl)-3-(2- (methylthio)-4-((3-oxo-3,4- dihydropyrido[2,3-b]pyrazin-8- yl)oxy)phenyl)urea |
| (ai) | 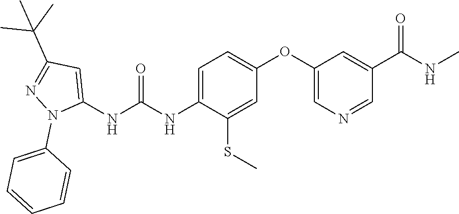 [SMILES: CNC(=O)c1cncc(Oc2ccc(NC(=O)Nc3cc(C(C)(C)C)nn3-c3ccccc3)c(SC)c2)c1] | 5-(4-(3-(3-(tert-butyl)-1-phenyl-1H- pyrazol-5-yl)ureido)-3- (methylthio)phenoxy)-N- methylnicotinamide |
| (aj) | 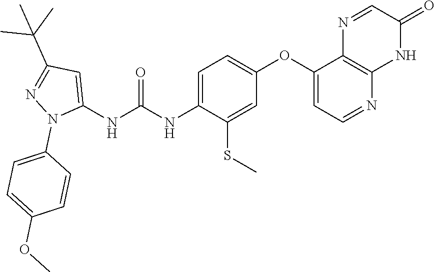 [SMILES: COc1ccc(-n2nc(C(C)(C)C)cc2NC(=O)Nc2ccc(Oc3ccnc4[nH]c(=O)cnc34)cc2SC)cc1] | 1-(3-(tert-butyl)-1-(4- methoxyphenyl)-1H-pyrazol-5-yl)-3- (2-(methylthio)-4-((3-oxo-3,4- dihydropyrido[2,3-b]pyrazin-8- yl)oxy)phenyl)urea |
| (ak) | 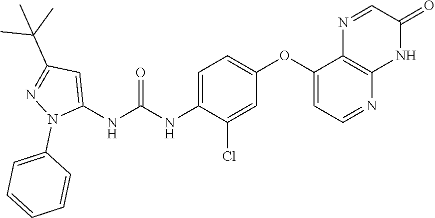 [SMILES: CC(C)(C)c1cc(NC(=O)Nc2ccc(Oc3ccnc4[nH]c(=O)cnc34)cc2Cl)n(-c2ccccc2)n1] | 1-(3-(tert-butyl)-1-phenyl-1H- pyrazol-5-yl)-3-(2-chloro-4-((3-oxo- 3,4-dihydropyrido[2,3-b]pyrazin-8- yl)oxy)phenyl)urea |
| (al) | 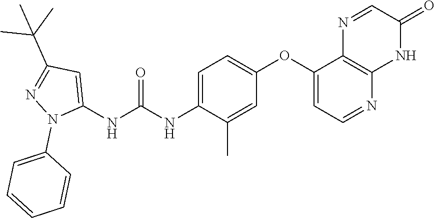 [SMILES: Cc1cc(Oc2ccnc3[nH]c(=O)cnc23)ccc1NC(=O)Nc1cc(C(C)(C)C)nn1-c1ccccc1] | 1-(3-(tert-butyl)-1-phenyl-1H- pyrazol-5-yl)-3-(2-methyl-4-((3-oxo- 3,4-dihydropyrido[2,3-b]pyrazin-8- yl)oxy)phenyl)urea |
| (am) | 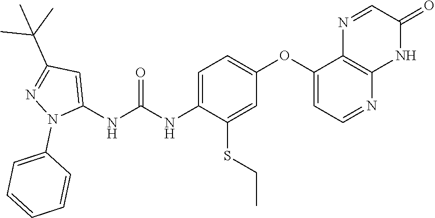 [SMILES: CCSc1cc(Oc2ccnc3[nH]c(=O)cnc23)ccc1NC(=O)Nc1cc(C(C)(C)C)nn1-c1ccccc1] | 1-(3-(tert-butyl)-1-phenyl-1H- pyrazol-5-yl)-3-(2-(ethylthio)-4-((3- oxo-3,4-dihydropyrido[2,3- b]pyrazin-8-yl)oxy)phenyl)urea |
| (an) | 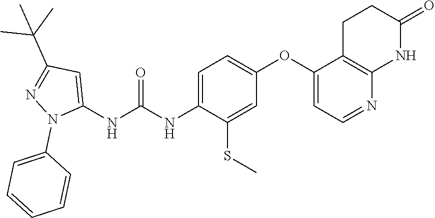 [SMILES: CSc1cc(Oc2ccnc3c2CCC(=O)N3)ccc1NC(=O)Nc1cc(C(C)(C)C)nn1-c1ccccc1] | 1-(3-(tert-butyl)-1-phenyl-1H- pyrazol-5-yl)-3-(2-(methylthio)-4- ((7-oxo-5,6,7,8-tetrahydro-1,8- naphthyridin-4-yl)oxy)phenyl)urea |
| (ao) | 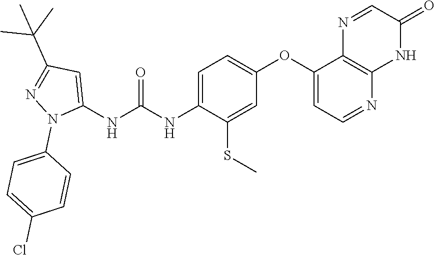 [SMILES: CSc1cc(Oc2ccnc3[nH]c(=O)cnc23)ccc1NC(=O)Nc1cc(C(C)(C)C)nn1-c1ccc(Cl)cc1] | 1-(3-(tert-butyl)-1-(4-chlorophenyl)- 1H-pyrazol-5-yl)-3-(2-(methylthio)- 4-((3-oxo-3,4-dihydropyrido[2,3- b]pyrazin-8-yl)oxy)phenyl)urea |
| (ap) | 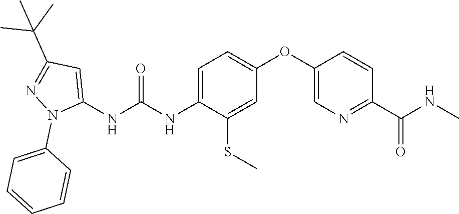 [SMILES: CNC(=O)c1ccc(Oc2ccc(NC(=O)Nc3cc(C(C)(C)C)nn3-c3ccccc3)c(SC)c2)cn1] | 5-(4-(3-(3-(tert-butyl)-1-phenyl-1H- pyrazol-5-yl)ureido)-3- (methylthio)phenoxy)-N- methylpicolinamide |
| (aq) | 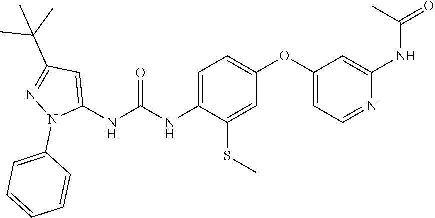 [SMILES: CSc1cc(Oc2ccnc(NC(C)=O)c2)ccc1NC(=O)Nc1cc(C(C)(C)C)nn1-c1ccccc1] | N-(4-(4-(3-(3-(tert-butyl)-1-phenyl- 1H-pyrazol-5-yl)ureido)-3- (methylthio)phenoxy)pyridin-2- yl)acetamide |
| (ar) | 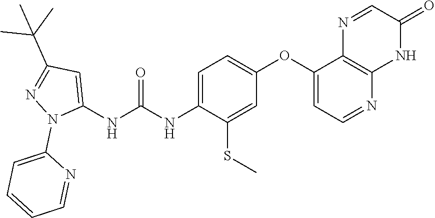 [SMILES: CSc1cc(Oc2ccnc3[nH]c(=O)cnc23)ccc1NC(=O)Nc1cc(C(C)(C)C)nn1-c1ccccn1] | 1-(3-(tert-butyl)-1-(pyridin-2-yl)-1H- pyrazol-5-yl)-3-(2-(methylthio)-4- ((3-oxo-3,4-dihydropyrido[2,3- b]pyrazin-8-yl)oxy)phenyl)urea |
| (as) | 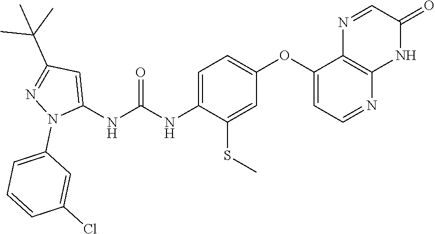 [SMILES: CSc1cc(Oc2ccnc3[nH]c(=O)cnc23)ccc1NC(=O)Nc1cc(C(C)(C)C)nn1-c1cccc(Cl)c1] | 1-(3-(tert-butyl)-1-(3-chlorophenyl)- 1H-pyrazol-5-yl)-3-(2-(methylthio)- 4-((3-oxo-3,4-dihydropyrido[2,3- b]pyrazin-8-yl)oxy)phenyl)urea |
| (at) | 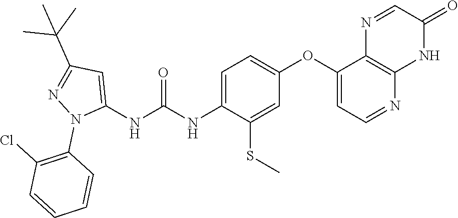 [SMILES: CSc1cc(Oc2ccnc3[nH]c(=O)cnc23)ccc1NC(=O)Nc1cc(C(C)(C)C)nn1-c1ccccc1Cl] | 1-(3-(tert-butyl)-1-(2-chlorophenyl)- 1H-pyrazol-5-yl)-3-(2-(methylthio)- 4-((3-oxo-3,4-dihydropyrido[2,3- b]pyrazin-8-yl)oxy)phenyl)urea |
| (au) | 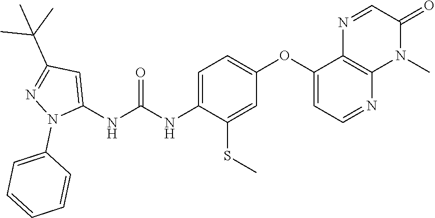 [SMILES: CSc1cc(Oc2ccnc3c2ncc(=O)n3C)ccc1NC(=O)Nc1cc(C(C)(C)C)nn1-c1ccccc1] | 1-(3-(tert-butyl)-1-phenyl-1H- pyrazol-5-yl)-3-(4-((4-methyl-3-oxo- 3,4-dihydropyrido[2,3-b]pyrazin-8- yl)oxy)-2-(methylthio)phenyl)urea |
| (av) | 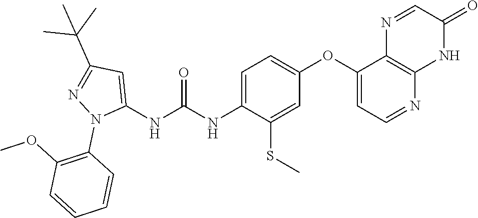 [SMILES: COc1ccccc1-n1nc(C(C)(C)C)cc1NC(=O)Nc1ccc(Oc2ccnc3[nH]c(=O)cnc23)cc1SC] | 1-(3-(tert-butyl)-1-(2- methoxyphenyl)-1H-pyrazol-5-yl)-3- (2-(methylthio)-4-((3-oxo-3,4- dihydropyrido[2,3-b]pyrazin-8- yl)oxy)phenyl)urea |
| (aw) | 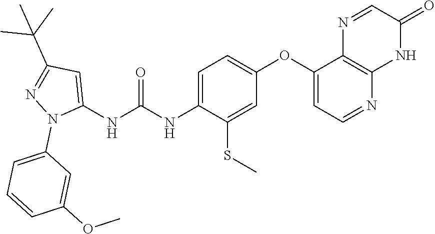 [SMILES: COc1cccc(-n2nc(C(C)(C)C)cc2NC(=O)Nc2ccc(Oc3ccnc4[nH]c(=O)cnc34)cc2SC)c1] | 1-(3-(tert-butyl)-1-(3- methoxyphenyl)-1H-pyrazol-5-yl)-3- (2-(methylthio)-4-((3-oxo-3,4- dihydropyrido[2,3-b]pyrazin-8- yl)oxy)phenyl)urea |
| (ax) | 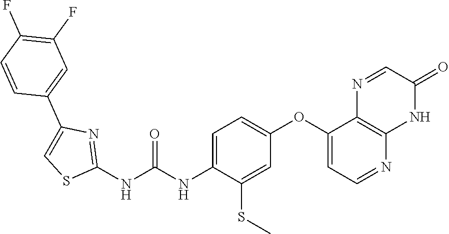 [SMILES: CSc1cc(Oc2ccnc3[nH]c(=O)cnc23)ccc1NC(=O)Nc1nc(-c2ccc(F)c(F)c2)cs1] | 1-(4-(3,4-difluorophenyl)thiazol-2- yl)-3-(2-(methylthio)-4-((3-oxo-3,4- dihydropyrido[2,3-b]pyrazin-8- yl)oxy)phenyl)urea |
| (ay) | 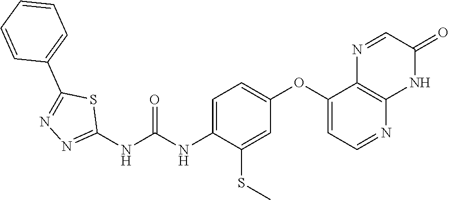 [SMILES: CSc1cc(Oc2ccnc3[nH]c(=O)cnc23)ccc1NC(=O)Nc1nnc(-c2ccccc2)s1] | 1-(2-(methylthio)-4-((3-oxo-3,4- dihydropyrido[2,3-b]pyrazin-8- yl)oxy)phenyl)-3-(5-phenyl-1,3,4- thiadiazol-2-yl)urea |
| (az) | 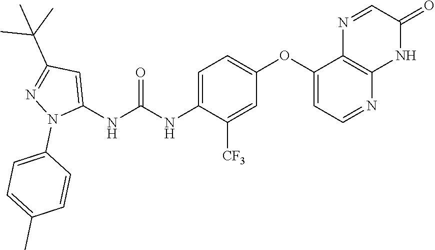 [SMILES: Cc1ccc(-n2nc(C(C)(C)C)cc2NC(=O)Nc2ccc(Oc3ccnc4[nH]c(=O)cnc34)cc2C(F)(F)F)cc1] | 1-(3-(tert-butyl)-1-(p-tolyl)-1H- pyrazol-5-yl)-3-(4-((3-oxo-3,4- dihydropyrido[2,3-b]pyrazin-8- yl)oxy)-2- (trifluoromethyl)phenyl)urea |
| (ba) | 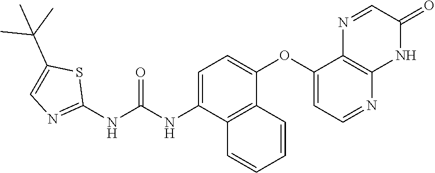 [SMILES: CC(C)(C)c1cnc(NC(=O)Nc2ccc(Oc3ccnc4[nH]c(=O)cnc34)c3ccccc23)s1] | 1-(5-(tert-butyl)thiazol-2-yl)-3-(4- ((3-oxo-3,4-dihydropyrido[2,3- b]pyrazin-8-yl)oxy)naphthalen-1- yl)urea |
| (bb) | 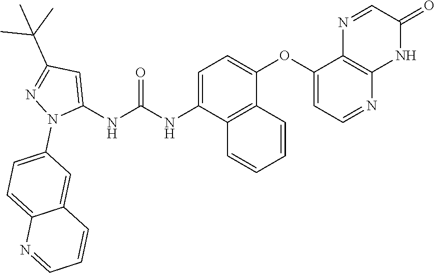 [SMILES: CC(C)(C)c1cc(NC(=O)Nc2ccc(Oc3ccnc4[nH]c(=O)cnc34)c3ccccc23)n(-c2ccc3ncccc3c2)n1] | 1-(3-(tert-butyl)-1-(quinolin-6-yl)- 1H-pyrazol-5-yl)-3-(4-((3-oxo-3,4- dihydropyrido[2,3-b]pyrazin-8- yl)oxy)naphthalen-1-yl)urea |
| (bc) | 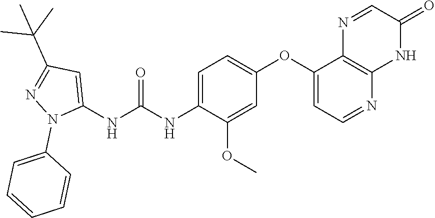 [SMILES: COc1cc(Oc2ccnc3[nH]c(=O)cnc23)ccc1NC(=O)Nc1cc(C(C)(C)C)nn1-c1ccccc1] | 1-(3-(tert-butyl)-1-phenyl-1H- pyrazol-5-yl)-3-(2-methoxy-4-((3- oxo-3,4-dihydropyrido[2,3- b]pyrazin-8-yl)oxy)phenyl)urea |
| (bd) | 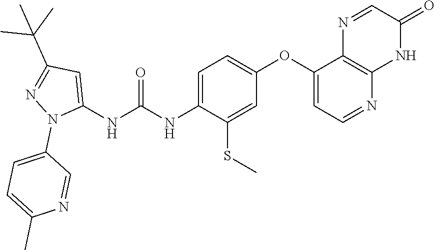 [SMILES: CSc1cc(Oc2ccnc3[nH]c(=O)cnc23)ccc1NC(=O)Nc1cc(C(C)(C)C)nn1-c1ccc(C)nc1] | 1-(3-(tert-butyl)-1-(6-methylpyridin- 3-yl)-1H-pyrazol-5-yl)-3-(2- (methylthio)-4-((3-oxo-3,4- dihydropyrido[2,3-b]pyrazin-8- yl)oxy)phenyl)urea |
| (be) | 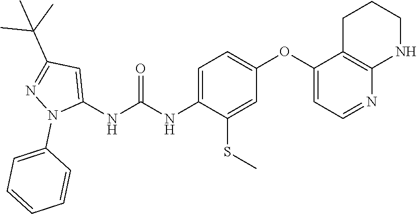 [SMILES: CSc1cc(Oc2ccnc3c2CCCN3)ccc1NC(=O)Nc1cc(C(C)(C)C)nn1-c1ccccc1] | 1-(3-(tert-butyl)-1-phenyl-1H- pyrazol-5-yl)-3-(2-(methylthio)-4- ((5,6,7,8-tetrahydro-1,8- naphthyridin-4-yl)oxy)phenyl)urea |
| (bf) | 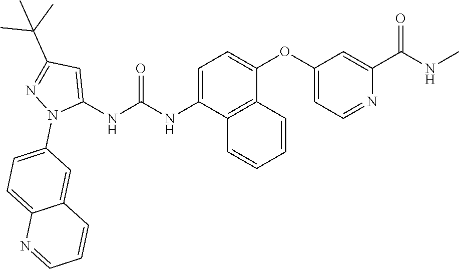 [SMILES: CNC(=O)c1cc(Oc2ccc(NC(=O)Nc3cc(C(C)(C)C)nn3-c3ccc4ncccc4c3)c3ccccc23)ccn1] | 4-((4-(3-(3-(tert-butyl)-1-(quinolin-6- yl)-1H-pyrazol-5- yl)ureido)naphthalen-1-yl)oxy)-N- methylpicolinamide |
| (bg) | 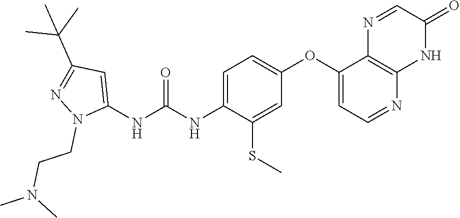 [SMILES: CSc1cc(Oc2ccnc3[nH]c(=O)cnc23)ccc1NC(=O)Nc1cc(C(C)(C)C)nn1CCN(C)C] | 1-(3-(tert-butyl)-1-(2- (dimethylamino)ethyl)-1H-pyrazol-5- yl)-3-(2-(methylthio)-4-((3-oxo-3,4- dihydropyrido[2,3-b]pyrazin-8- yl)oxy)phenyl)urea |
| (bh) | 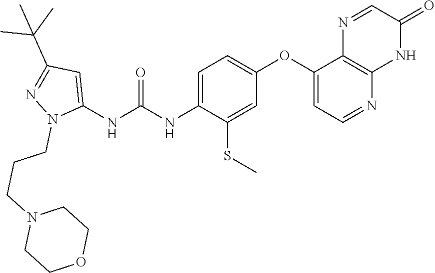 [SMILES: CSc1cc(Oc2ccnc3[nH]c(=O)cnc23)ccc1NC(=O)Nc1cc(C(C)(C)C)nn1CCCN1CCOCC1] | 1-(3-(tert-butyl)-1-(3- morpholinopropyl)-1H-pyrazol-5-yl)- 3-(2-(methylthio)-4-((3-oxo-3,4- dihydropyrido[2,3-b]pyrazin-8- yl)oxy)phenyl)urea |
| (bi) | 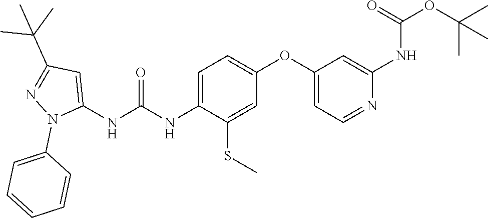 [SMILES: CSc1cc(Oc2ccnc(NC(=O)OC(C)(C)C)c2)ccc1NC(=O)Nc1cc(C(C)(C)C)nn1-c1ccccc1] | tert-butyl (4-(4-(3-(3-(tert-butyl)- phenyl-1H-pyrazol-5-yl)ureido)-3- (methylthio)phenoxy)pyridin-2- yl)carbamate |
| (bj) | 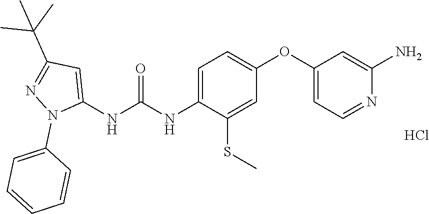 [SMILES: CSc1cc(Oc2ccnc(N)c2)ccc1NC(=O)Nc1cc(C(C)(C)C)nn1-c1ccccc1 ; Cl] | 1-(4-((2-aminopyridin-4-yl)oxy)-2- (methylthio)phenyl)-3-(3-(tert-butyl)- 1-phenyl-1H-pyrazol-5-yl)urea hydrochloride |
| (bk) | 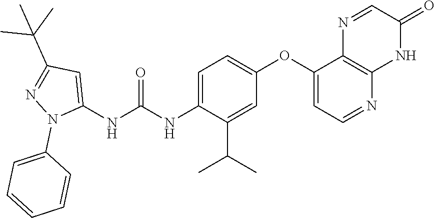 [SMILES: CC(C)c1cc(Oc2ccnc3[nH]c(=O)cnc23)ccc1NC(=O)Nc1cc(C(C)(C)C)nn1-c1ccccc1] | 1-(3-(tert-butyl)-1-phenyl-1H- pyrazol-5-yl)-3-(2-isopropyl-4-((3- oxo-3,4-dihydropyrido[2,3- b]pyrazin-8-yl)oxy)phenyl)urea |
| (bl) | 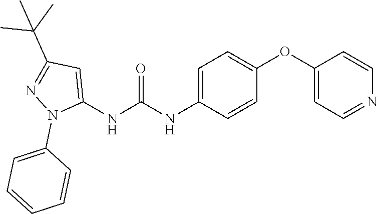 [SMILES: CC(C)(C)c1cc(NC(=O)Nc2ccc(Oc3ccncc3)cc2)n(-c2ccccc2)n1] | 1-(3-(tert-butyl)-1-phenyl-1H- pyrazol-5-yl)-3-(4-(pyridin-4- yloxy)phenyl)urea |
| (bm) | 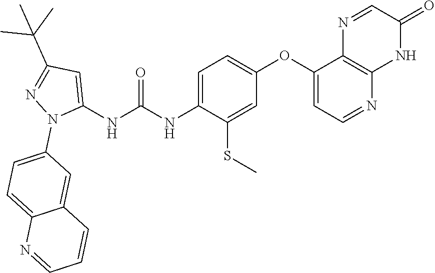 [SMILES: CSc1cc(Oc2ccnc3[nH]c(=O)cnc23)ccc1NC(=O)Nc1cc(C(C)(C)C)nn1-c1ccc2ncccc2c1] | 1-(3-(tert-butyl)-1-(quinolin-6-yl)- 1H-pyrazol-5-yl)-3-(2-(methylthio)- 4-((3-oxo-3,4-dihydropyrido[2,3- b]pyrazin-8-yl)oxy)phenyl)urea |
| (bn) | 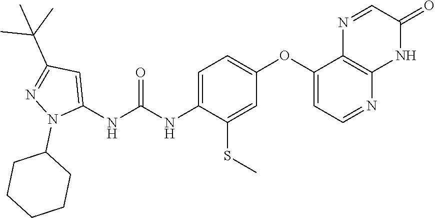 [SMILES: CSc1cc(Oc2ccnc3[nH]c(=O)cnc23)ccc1NC(=O)Nc1cc(C(C)(C)C)nn1C1CCCCC1] | 1-(3-(tert-butyl)-1-cyclohexyl-1H- pyrazol-5-yl)-3-(2-(methylthio)-4- ((3-oxo-3,4-dihydropyrido[2,3- b]pyrazin-8-yl)oxy)phenyl)urea |
| (bo) | 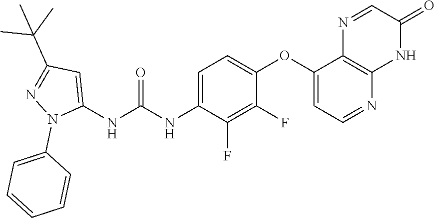 [SMILES: CC(C)(C)c1cc(NC(=O)Nc2ccc(Oc3ccnc4[nH]c(=O)cnc34)c(F)c2F)n(-c2ccccc2)n1] | 1-(3-(tert-butyl)-1-phenyl-1H- pyrazol-5-yl)-3-(2,3-difluoro-4-((3- oxo-3,4-dihydropyrido[2,3- b]pyrazin-8-yl)oxy)phenyl)urea |
| (bp) | 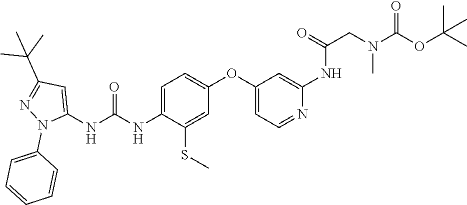 [SMILES: CSc1cc(Oc2ccnc(NC(=O)CN(C)C(=O)OC(C)(C)C)c2)ccc1NC(=O)Nc1cc(C(C)(C)C)nn1-c1ccccc1] | tert-butyl (2-((4-(4-(3-(3-(tert-butyl)- 1-phenyl-1H-pyrazol-5-yl)ureido)-3- (methylthio)phenoxy)pyridin-2- yl)amino)-2- oxoethyl)(methyl)carbamate |
| (bq) | 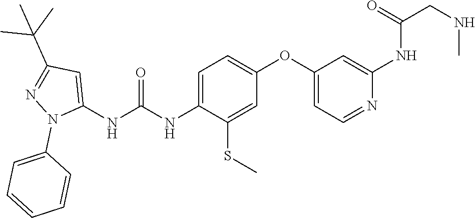 [SMILES: CNCC(=O)Nc1cc(Oc2ccc(NC(=O)Nc3cc(C(C)(C)C)nn3-c3ccccc3)c(SC)c2)ccn1] | N-(4-(4-(3-(3-(tert-butyl)-1-phenyl- 1H-pyrazol-5-yl)ureido)-3- (methylthio)phenoxy)pyridin-2-yl)-2- (methylamino)acetamide |
| (br) | 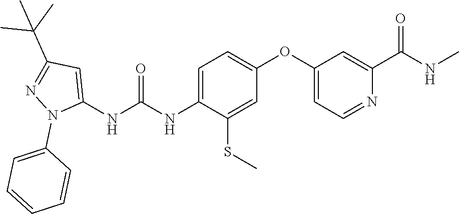 [SMILES: CNC(=O)c1cc(Oc2ccc(NC(=O)Nc3cc(C(C)(C)C)nn3-c3ccccc3)c(SC)c2)ccn1] | 4-(4-(3-(3-(tert-butyl)-1-phenyl-1H- pyrazol-5-yl)ureido)-3- (methylthio)phenoxy)-N- methylpicolinamide |
| (bs) | 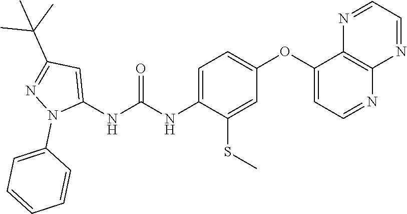 [SMILES: CSc1cc(Oc2ccnc3nccnc23)ccc1NC(=O)Nc1cc(C(C)(C)C)nn1-c1ccccc1] | 1-(3-(tert-butyl)-1-phenyl-1H- pyrazol-5-yl)-3-(2-(methylthio)-4- (pyrido[2,3-b]pyrazin-8- yloxy)phenyl)urea |
| (bt) | 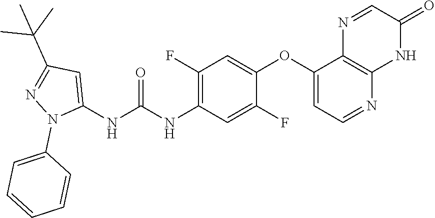 [SMILES: CC(C)(C)c1cc(NC(=O)Nc2cc(F)c(Oc3ccnc4[nH]c(=O)cnc34)cc2F)n(-c2ccccc2)n1] | 1-(3-(tert-butyl)-1-phenyl-1H- pyrazol-5-yl)-3-(2,5-difluoro-4-((3- oxo-3,4-dihydropyrido[2,3- b]pyrazin-8-yl)oxy)phenyl)urea |
| (bu) | 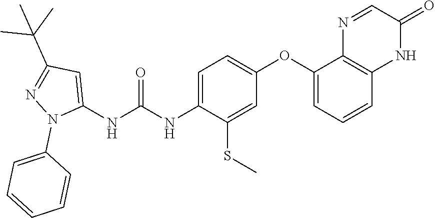 [SMILES: CSc1cc(Oc2cccc3[nH]c(=O)cnc23)ccc1NC(=O)Nc1cc(C(C)(C)C)nn1-c1ccccc1] | 1-(3-(tert-butyl)-1-phenyl-1H- pyrazol-5-yl)-3-(2-(methylthio)-4- ((2-oxo-1,2-dihydroquinoxalin-5- yl)oxy)phenyl)urea |
| (bv) | 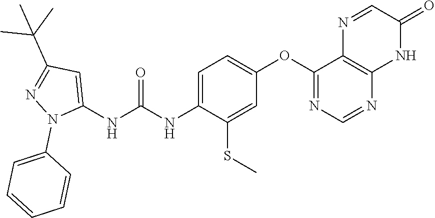 [SMILES: CSc1cc(Oc2ncnc3[nH]c(=O)cnc23)ccc1NC(=O)Nc1cc(C(C)(C)C)nn1-c1ccccc1] | 1-(3-(tert-butyl)-1-phenyl-1H- pyrazol-5-yl)-3-(2-(methylthio)-4- ((7-oxo-7,8-dihydropteridin-4- yl)oxy)phenyl)urea |
| (bw) | 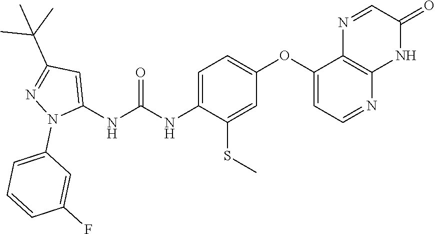 [SMILES: CSc1cc(Oc2ccnc3[nH]c(=O)cnc23)ccc1NC(=O)Nc1cc(C(C)(C)C)nn1-c1cccc(F)c1] | 1-(3-(tert-butyl)-1-(3-fluorophenyl)- 1H-pyrazol-5-yl)-3-(2-(methylthio)- 4-((3-oxo-3,4-dihydropyrido[2,3- b]pyrazin-8-yl)oxy)phenyl)urea |
| (bx) | 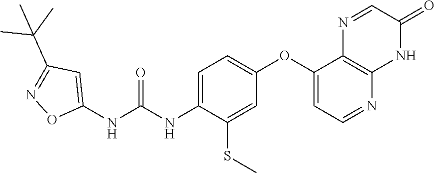 [SMILES: CSc1cc(Oc2ccnc3[nH]c(=O)cnc23)ccc1NC(=O)Nc1cc(C(C)(C)C)no1] | 1-(3-(tert-butyl)isoxazol-5-yl)-3-(2- (methylthio)-4-((3-oxo-3,4- dihydropyrido[2,3-b]pyrazin-8- yl)oxy)phenyl)urea |
| (by) | 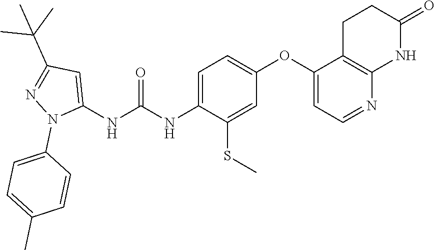 [SMILES: CSc1cc(Oc2ccnc3c2CCC(=O)N3)ccc1NC(=O)Nc1cc(C(C)(C)C)nn1-c1ccc(C)cc1] | 1-(3-(tert-butyl)-1-(p-tolyl)-1H- pyrazol-5-yl)-3-(2-(methylthio)-4- ((7-oxo-5,6,7,8-tetrahydro-1,8- naphthyridin-4-yl)oxy)phenyl)urea |
| (bz) | 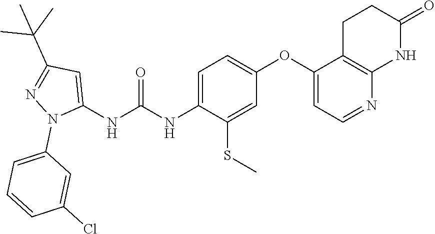 [SMILES: CSc1cc(Oc2ccnc3c2CCC(=O)N3)ccc1NC(=O)Nc1cc(C(C)(C)C)nn1-c1cccc(Cl)c1] | 1-(3-(tert-butyl)-1-(3-chlorophenyl)- 1H-pyrazol-5-yl)-3-(2-(methylthio)- 4-((7-oxo-5,6,7,8-tetrahydro-1,8- naphthyridin-4-yl)oxy)phenyl)urea |
| (ca) | 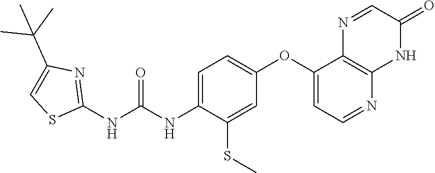 [SMILES: CSc1cc(Oc2ccnc3[nH]c(=O)cnc23)ccc1NC(=O)Nc1nc(C(C)(C)C)cs1] | 1-(4-(tert-butyl)thiazol-2-yl)-3-(2- (methylthio)-4-((3-oxo-3,4- dihydropyrido[2,3-b]pyrazin-8- yl)oxy)phenyl)urea |
| (cb) | 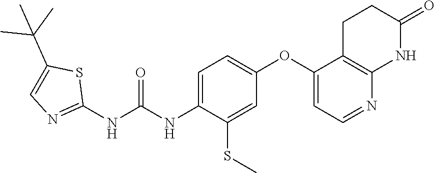 [SMILES: CSc1cc(Oc2ccnc3c2CCC(=O)N3)ccc1NC(=O)Nc1ncc(C(C)(C)C)s1] | 1-(5-(tert-butyl)thiazol-2-yl)-3-(2- (methylthio)-4-((7-oxo-5,6,7,8- tetrahydro-1,8-naphthyridin-4- yl)oxy)phenyl)urea |
| (cc) | 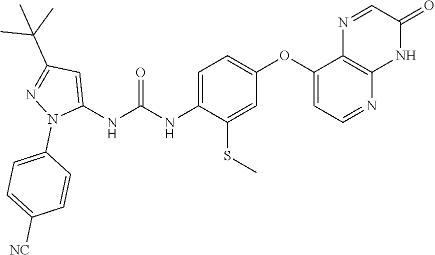 [SMILES: CSc1cc(Oc2ccnc3[nH]c(=O)cnc23)ccc1NC(=O)Nc1cc(C(C)(C)C)nn1-c1ccc(C#N)cc1] | 1-(3-(tert-butyl)-1-(4-cyanophenyl)- 1H-pyrazol-5-yl)-3-(2-(methylthio)- 4-((3-oxo-3,4-dihydropyrido[2,3- b]pyrazin-8-yl)oxy)phenyl)urea |
| (cd) | 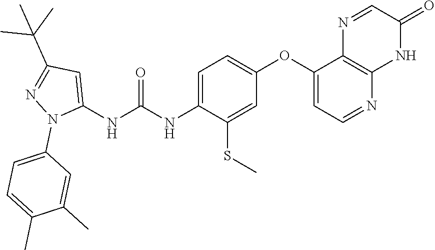 [SMILES: CSc1cc(Oc2ccnc3[nH]c(=O)cnc23)ccc1NC(=O)Nc1cc(C(C)(C)C)nn1-c1ccc(C)c(C)c1] | 1-(3-(tert-butyl)-1-(3,4- dimethylphenyl)-1H-pyrazol-5-yl)-3- (2-(methylthio)-4-((3-oxo-3,4- dihydropyrido[2,3-b]pyrazin-8- yl)oxy)phenyl)urea |
| (ce) | 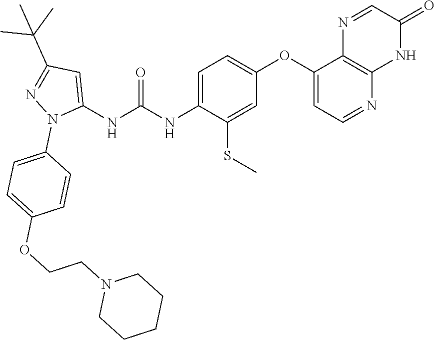 [SMILES: CSc1cc(Oc2ccnc3[nH]c(=O)cnc23)ccc1NC(=O)Nc1cc(C(C)(C)C)nn1-c1ccc(OCCN2CCCCC2)cc1] | 1-(3-(tert-butyl)-1-(4-(2-(piperidin-1- yl)ethoxy)phenyl)-1H-pyrazol-5-yl)- 3-(2-(methylthio)-4-((3-oxo-3,4- dihydropyrido[2,3-b]pyrazin-8- yl)oxy)phenyl)urea |
| (cf) | 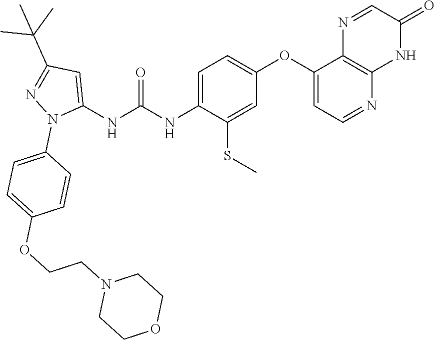 [SMILES: CSc1cc(Oc2ccnc3[nH]c(=O)cnc23)ccc1NC(=O)Nc1cc(C(C)(C)C)nn1-c1ccc(OCCN2CCOCC2)cc1] | 1-(3-(tert-butyl)-1-(4-(2- morpholinoethoxy)phenyl)-1H- pyrazol-5-yl)-3-(2-(methylthio)-4- ((3-oxo-3,4-dihydropyrido[2,3- b]pyrazin-8-yl)oxy)phenyl)urea |
| (cg) | 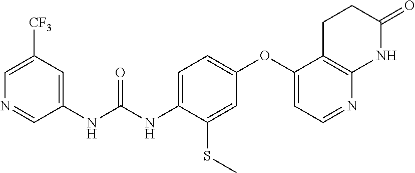 [SMILES: CSc1cc(Oc2ccnc3c2CCC(=O)N3)ccc1NC(=O)Nc1cncc(C(F)(F)F)c1] | 1-(2-(methylthio)-4-((7-oxo-5,6,7,8- tetrahydro-1,8-naphthyridin-4- yl)oxy)phenyl)-3-(5- (trifluoromethyl)pyridin-3-yl)urea |
| (ch) | 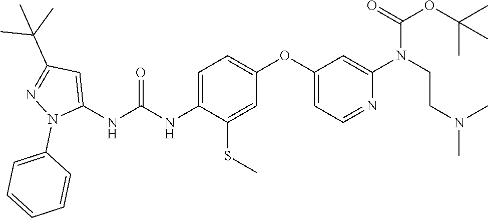 [SMILES: CSc1cc(Oc2ccnc(N(CCN(C)C)C(=O)OC(C)(C)C)c2)ccc1NC(=O)Nc1cc(C(C)(C)C)nn1-c1ccccc1] | tert-butyl (4-(4-(3-(3-(tert-butyl)-1- phenyl-1H-pyrazol-5-yl)ureido)-3- (methylthio)phenoxy)pyridin-2-yl)(2- (dimethylamino)ethyl)carbamate |
| (ci) | 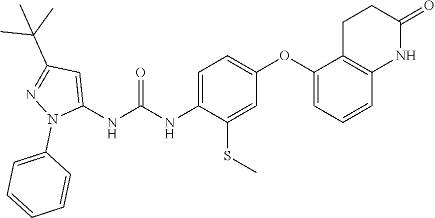 [SMILES: CSc1cc(Oc2cccc3c2CCC(=O)N3)ccc1NC(=O)Nc1cc(C(C)(C)C)nn1-c1ccccc1] | 1-(3-(tert-butyl)-1-phenyl-1H- pyrazol-5-yl)-3-(2-(methylthio)-4- ((2-oxo-1,2,3,4-tetrahydroquinolin-5- yl)oxy)phenyl)urea |
| (cj) | 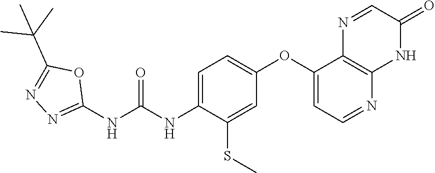 [SMILES: CSc1cc(Oc2ccnc3[nH]c(=O)cnc23)ccc1NC(=O)Nc1nnc(C(C)(C)C)o1] | 1-(5-(tert-butyl)-1,3,4-oxadiazol-2- yl)-3-(2-(methylthio)-4-((3-oxo-3,4- dihydropyrido[2,3-b]pyrazin-8- yl)oxy)phenyl)urea |
| (ck) | 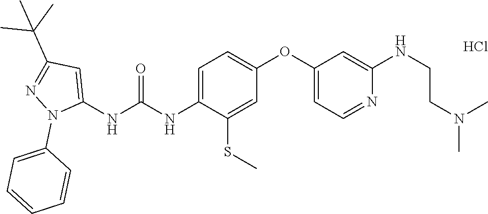 [SMILES: CSc1cc(Oc2ccnc(NCCN(C)C)c2)ccc1NC(=O)Nc1cc(C(C)(C)C)nn1-c1ccccc1 ; Cl] | 1-(3-(tert-butyl)-1-phenyl-1H- pyrazol-5-yl)-3-(4-((2-((2- (dimethylamino)ethyl)amino)pyridin- 4-yl)oxy)-2-(methylthio)phenyl)urea hydrochloride |
| (cl) | 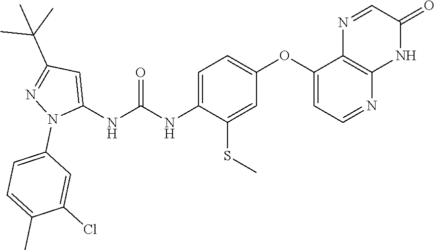 [SMILES: CSc1cc(Oc2ccnc3[nH]c(=O)cnc23)ccc1NC(=O)Nc1cc(C(C)(C)C)nn1-c1ccc(C)c(Cl)c1] | 1-(3-(tert-butyl)-1-(3-chloro-4- methylphenyl)-1H-pyrazol-5-yl)-3- (2-(methylthio)-4-((3-oxo-3,4- dihydropyrido[2,3-b]pyrazin-8- yl)oxy)phenyl)urea |
| (cm) | 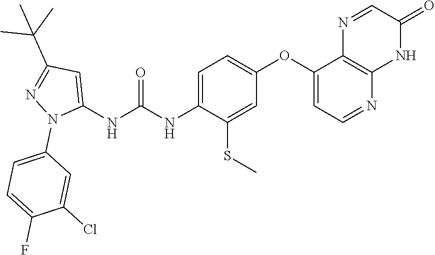 [SMILES: CSc1cc(Oc2ccnc3[nH]c(=O)cnc23)ccc1NC(=O)Nc1cc(C(C)(C)C)nn1-c1ccc(F)c(Cl)c1] | 1-(3-(tert-butyl)-1-(3-chloro-4- fluorophenyl)-1H-pyrazol-5-yl)-3-(2- (methylthio)-4-((3-oxo-3,4- dihydropyrido[2,3-b]pyrazin-8- yl)oxy)phenyl)urea |
| (cn) | 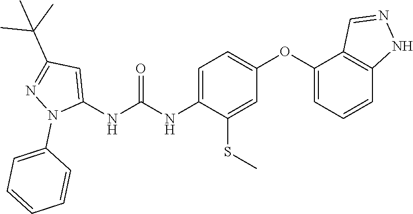 [SMILES: CSc1cc(Oc2cccc3[nH]ncc23)ccc1NC(=O)Nc1cc(C(C)(C)C)nn1-c1ccccc1] | 1-(4-((1H-indazol-4-yl)oxy)-2- (methylthio)phenyl)-3-(3-(tert-butyl)- 1-phenyl-1H-pyrazol-5-yl)urea |
| (co) | 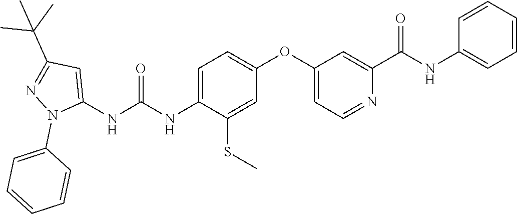 [SMILES: CSc1cc(Oc2ccnc(C(=O)Nc3ccccc3)c2)ccc1NC(=O)Nc1cc(C(C)(C)C)nn1-c1ccccc1] | 4-(4-(3-(3-(tert-butyl)-1-phenyl-1H- pyrazol-5-yl)ureido)-3- (methylthio)phenoxy)-N- phenylpicolinamide |
| (cp) | 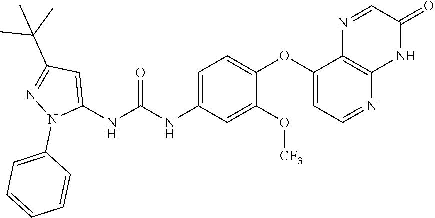 [SMILES: CC(C)(C)c1cc(NC(=O)Nc2ccc(Oc3ccnc4[nH]c(=O)cnc34)c(OC(F)(F)F)c2)n(-c2ccccc2)n1] | 1-(3-(tert-butyl)-1-phenyl-1H- pyrazol-5-yl)-3-(4-((3-oxo-3,4- dihydropyrido[2,3-b]pyrazin-8- yl)oxy)-3- (trifluoromethoxy)phenyl)urea |
| (cq) | 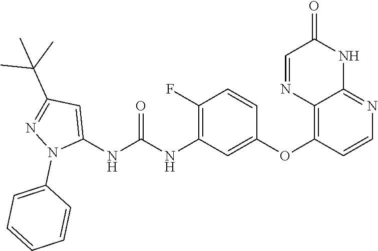 [SMILES: CC(C)(C)c1cc(NC(=O)Nc2cc(Oc3ccnc4[nH]c(=O)cnc34)ccc2F)n(-c2ccccc2)n1] | 1-(3-(tert-butyl)-1-phenyl-1H- pyrazol-5-yl)-3-(2-fluoro-5-((3-oxo- 3,4-dihydropyrido[2,3-b]pyrazin-8- yl)oxy)phenyl)urea |
| (cr) | 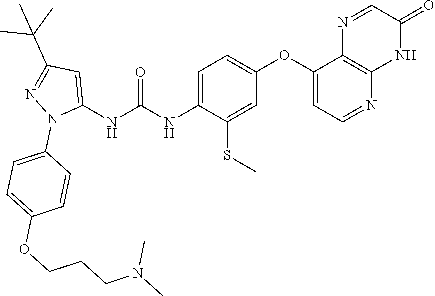 [SMILES: CSc1cc(Oc2ccnc3[nH]c(=O)cnc23)ccc1NC(=O)Nc1cc(C(C)(C)C)nn1-c1ccc(OCCCN(C)C)cc1] | 1-(3-(tert-butyl)-1-(4-(3- (dimethylamino)propoxy)phenyl)- 1H-pyrazol-5-yl)-3-(2-(methylthio)- 4-((3-oxo-3,4-dihydropyrido[2,3- b]pyrazin-8-yl)oxy)phenyl)urea |
| (cs) | 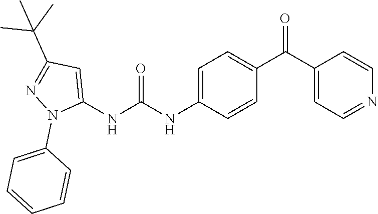 [SMILES: CC(C)(C)c1cc(NC(=O)Nc2ccc(C(=O)c3ccncc3)cc2)n(-c2ccccc2)n1] | 1-(3-(tert-butyl)-1-phenyl-1H- pyrazol-5-yl)-3-(4- isonicotinoylphenyl)urea |
| (ct) | 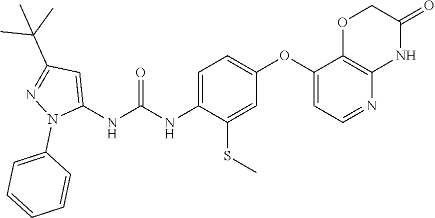 [SMILES: CSc1cc(Oc2ccnc3c2OCC(=O)N3)ccc1NC(=O)Nc1cc(C(C)(C)C)nn1-c1ccccc1] | 1-(3-(tert-butyl)-1-phenyl-1H- pyrazol-5-yl)-3-(2-(methylthio)-4- ((3-oxo-3,4-dihydro-2H-pyrido[3,2- b][1,4]oxazin-8-yl)oxy)phenyl)urea |
| (cu) | 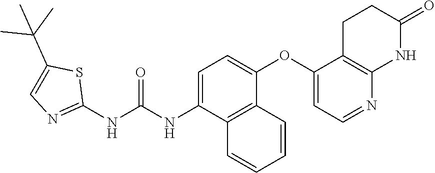 [SMILES: CC(C)(C)c1cnc(NC(=O)Nc2ccc(Oc3ccnc4c3CCC(=O)N4)c3ccccc23)s1] | 1-(5-(tert-butyl)thiazol-2-yl)-3-(4- ((7-oxo-5,6,7,8-tetrahydro-1,8- naphthyridin-4-yl)oxy)naphthalen-1- yl)urea |
| (cv) | 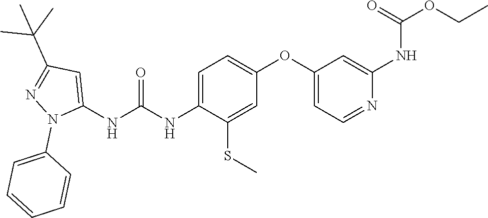 [SMILES: CCOC(=O)Nc1cc(Oc2ccc(NC(=O)Nc3cc(C(C)(C)C)nn3-c3ccccc3)c(SC)c2)ccn1] | ethyl (4-(4-(3-(3-(tert-butyl)- phenyl-1H-pyrazol-5-yl)ureido)-3- (methylthio)phenoxy)pyridin-2- yl)carbamate |
| (cw) | 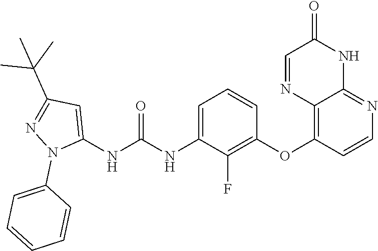 [SMILES: CC(C)(C)c1cc(NC(=O)Nc2cccc(Oc3ccnc4[nH]c(=O)cnc34)c2F)n(-c2ccccc2)n1] | 1-(3-(tert-butyl)-1-phenyl-1H- pyrazol-5-yl)-3-(2-fluoro-3-((3-oxo- 3,4-dihydropyrido[2,3-b]pyrazin-8- yl)oxy)phenyl)urea |
| (cx) | 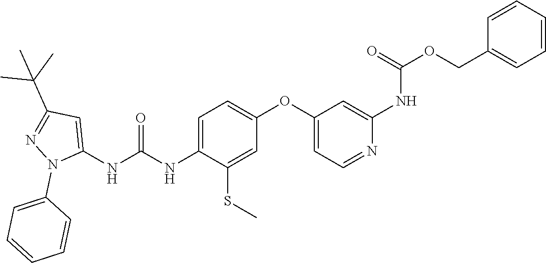 [SMILES: CSc1cc(Oc2ccnc(NC(=O)OCc3ccccc3)c2)ccc1NC(=O)Nc1cc(C(C)(C)C)nn1-c1ccccc1] | benzyl (4-(4-(3-(3-(tert-butyl)- phenyl-1H-pyrazol-5-yl)ureido)-3- (methylthio)phenoxy)pyridin-2- yl)carbamate |
| (cy) | 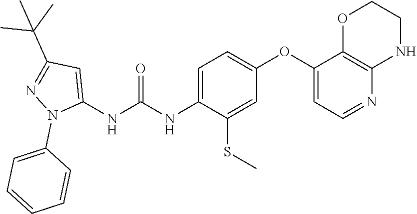 [SMILES: CSc1cc(Oc2ccnc3c2OCCN3)ccc1NC(=O)Nc1cc(C(C)(C)C)nn1-c1ccccc1] | 1-(3-(tert-butyl)-1-phenyl-1H- pyrazol-5-yl)-3-(4-((3,4-dihydro-2H- pyrido[3,2-b][1,4]oxazin-8-yl)oxy)- 2-(methylthio)phenyl)urea |
| (cz) | 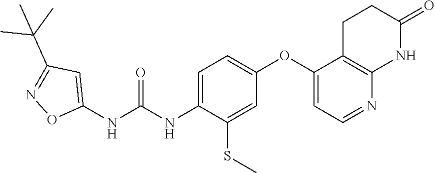 [SMILES: CSc1cc(Oc2ccnc3c2CCC(=O)N3)ccc1NC(=O)Nc1cc(C(C)(C)C)no1] | 1-(3-(tert-butyl)isoxazol-5-yl)-3-(2- (methylthio)-4-((7-oxo-5,6,7,8- tetrahydro-1,8-naphthyridin-4- yl)oxy)phenyl)urea |
| (da) | 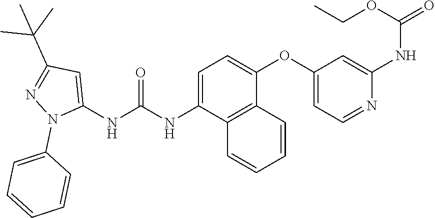 [SMILES: CCOC(=O)Nc1cc(Oc2ccc(NC(=O)Nc3cc(C(C)(C)C)nn3-c3ccccc3)c3ccccc23)ccn1] | ethyl (4-((4-(3-(3-(tert-butyl)- phenyl-1H-pyrazol-5- yl)ureido)naphthalen-1- yl)oxy)pyridin-2-yl)carbamate |
| (db) | 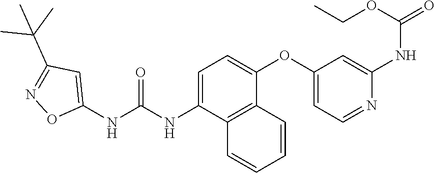 [SMILES: CCOC(=O)Nc1cc(Oc2ccc(NC(=O)Nc3cc(C(C)(C)C)no3)c3ccccc23)ccn1] | ethyl (4-((4-(3-(3-(tert- butyl)isoxazol-5- yl)ureido)naphthalen-1- yl)oxy)pyridin-2-yl)carbamate |
| (dc) | 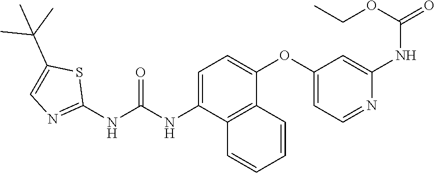 [SMILES: CCOC(=O)Nc1cc(Oc2ccc(NC(=O)Nc3ncc(C(C)(C)C)s3)c3ccccc23)ccn1] | ethyl (4-((4-(3-(5-(tert-butyl)thiazol- 2-yl)ureido)naphthalen-1- yl)oxy)pyridin-2-yl)carbamate |
| (dd) | 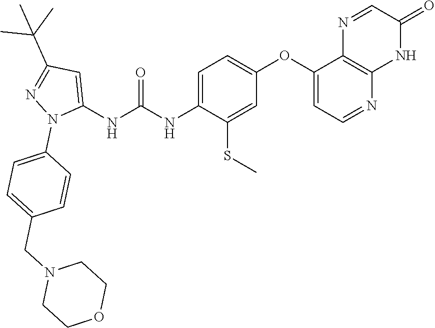 [SMILES: CSc1cc(Oc2ccnc3[nH]c(=O)cnc23)ccc1NC(=O)Nc1cc(C(C)(C)C)nn1-c1ccc(CN2CCOCC2)cc1] | 1-(3-(tert-butyl)-1-(4- (morpholinomethyl)phenyl)-1H- pyrazol-5-yl)-3-(2-(methylthio)-4- ((3-oxo-3,4-dihydropyrido[2,3- b]pyrazin-8-yl)oxy)phenyl)urea |
| (de) | 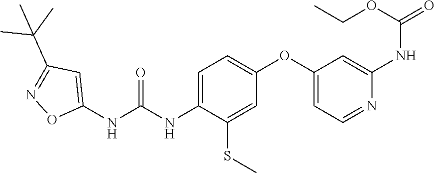 [SMILES: CCOC(=O)Nc1cc(Oc2ccc(NC(=O)Nc3cc(C(C)(C)C)no3)c(SC)c2)ccn1] | ethyl (4-(4-(3-(3-(tert-butyl)isoxazol- 5-yl)ureido)-3- (methylthio)phenoxy)pyridin-2- yl)carbamate |
| (df) | 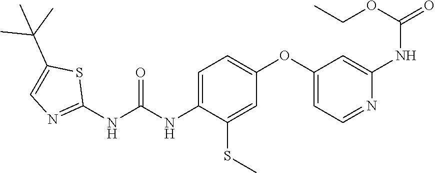 [SMILES: CCOC(=O)Nc1cc(Oc2ccc(NC(=O)Nc3ncc(C(C)(C)C)s3)c(SC)c2)ccn1] | ethyl (4-(4-(3-(5-(tert-butyl)thiazol- 2-yl)ureido)-3- (methylthio)phenoxy)pyridin-2- yl)carbamate |
| (dg) | 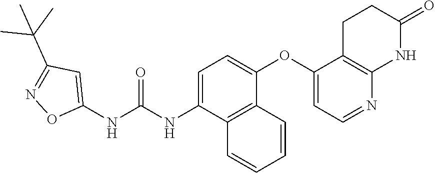 [SMILES: CC(C)(C)c1cc(NC(=O)Nc2ccc(Oc3ccnc4c3CCC(=O)N4)c3ccccc23)on1] | 1-(3-(tert-butyl)isoxazol-5-yl)-3-(4- ((7-oxo-5,6,7,8-tetrahydro-1,8- naphthyridin-4-yl)oxy)naphthalen-1- yl)urea |
| (dh) | 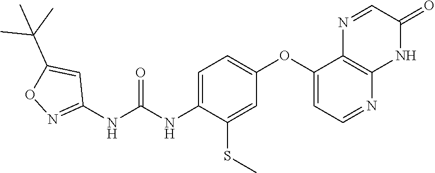 [SMILES: CSc1cc(Oc2ccnc3[nH]c(=O)cnc23)ccc1NC(=O)Nc1cc(C(C)(C)C)on1] | 1-(5-(tert-butyl)isoxazol-3-yl)-3-(2- (methylthio)-4-((3-oxo-3,4- dihydropyrido[2,3-b]pyrazin-8- yl)oxy)phenyl)urea |
| (di) | 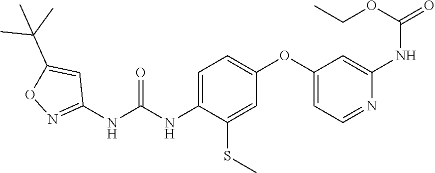 [SMILES: CCOC(=O)Nc1cc(Oc2ccc(NC(=O)Nc3cc(C(C)(C)C)on3)c(SC)c2)ccn1] | ethyl (4-(4-(3-(5-(tert-butyl)isoxazol- 3-yl)ureido)-3- (methylthio)phenoxy)pyridin-2- yl)carbamate |
| (dj) | 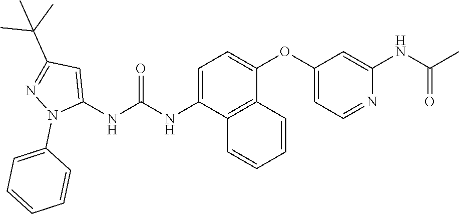 [SMILES: CC(=O)Nc1cc(Oc2ccc(NC(=O)Nc3cc(C(C)(C)C)nn3-c3ccccc3)c3ccccc23)ccn1] | N-(4-((4-(3-(3-(tert-butyl)-1-phenyl- 1H-pyrazol-5-yl)ureido)naphthalen- 1-yl)oxy)pyridin-2-yl)acetamide |
| (dk) | 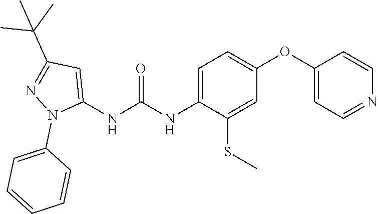 [SMILES: CSc1cc(Oc2ccncc2)ccc1NC(=O)Nc1cc(C(C)(C)C)nn1-c1ccccc1] | 1-(3-(tert-butyl)-1-phenyl-1H- pyrazol-5-yl)-3-(2-(methylthio)-4- (pyridin-4-yloxy)phenyl)urea |
| (dl) | 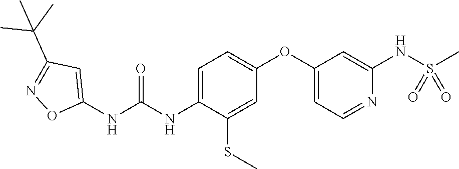 [SMILES: CSc1cc(Oc2ccnc(NS(C)(=O)=O)c2)ccc1NC(=O)Nc1cc(C(C)(C)C)no1] | N-(4-(4-(3-(3-(tert-butyl)isoxazol-5- yl)ureido)-3- (methylthio)phenoxy)pyridin-2- yl)methanesulfonamide |
| (dm) | 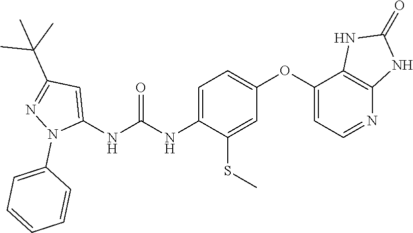 [SMILES: CSc1cc(Oc2ccnc3[nH]c(=O)[nH]c23)ccc1NC(=O)Nc1cc(C(C)(C)C)nn1-c1ccccc1] | 1-(3-(tert-butyl)-1-phenyl-1H- pyrazol-5-yl)-3-(2-(methylthio)-4- ((2-oxo-2,3-dihydro-1H-imidazo[4,5- b]pyridin-7-yl)oxy)phenyl)urea |
| (dn) | 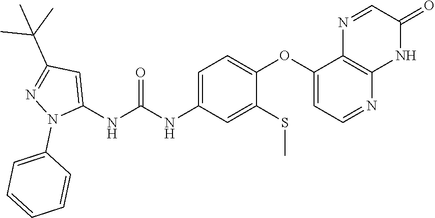 [SMILES: CSc1cc(NC(=O)Nc2cc(C(C)(C)C)nn2-c2ccccc2)ccc1Oc1ccnc2[nH]c(=O)cnc12] | 1-(3-(tert-butyl)-1-phenyl-1H- pyrazol-5-yl)-3-(3-(methylthio)-4- ((3-oxo-3,4-dihydropyrido[2,3- b]pyrazin-8-yl)oxy)phenyl)urea |
| (do) | 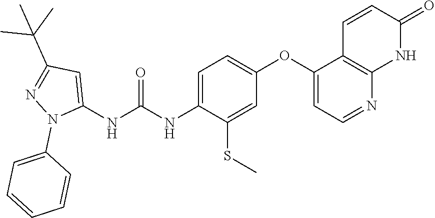 [SMILES: CSc1cc(Oc2ccnc3[nH]c(=O)ccc23)ccc1NC(=O)Nc1cc(C(C)(C)C)nn1-c1ccccc1] | 1-(3-(tert-butyl)-1-phenyl-1H- pyrazol-5-yl)-3-(2-(methylthio)-4- ((7-oxo-7,8-dihydro-1,8- naphthyridin-4-yl)oxy)phenyl)urea |
| (dp) | 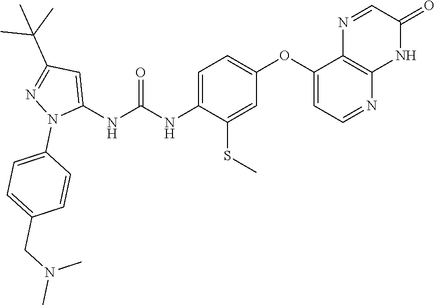 [SMILES: CSc1cc(Oc2ccnc3[nH]c(=O)cnc23)ccc1NC(=O)Nc1cc(C(C)(C)C)nn1-c1ccc(CN(C)C)cc1] | 1-(3-(tert-butyl)-1-(4- ((dimethylamino)methyl)phenyl)-1H- pyrazol-5-yl)-3-(2-(methylthio)-4- ((3-oxo-3,4-dihydropyrido[2,3- b]pyrazin-8-yl)oxy)phenyl)urea |
| (dq) | 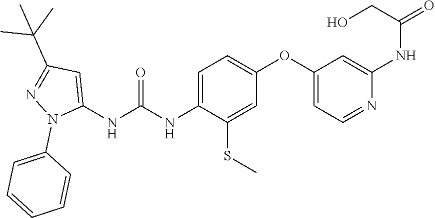 [SMILES: CSc1cc(Oc2ccnc(NC(=O)CO)c2)ccc1NC(=O)Nc1cc(C(C)(C)C)nn1-c1ccccc1] | N-(4-(4-(3-(3-(tert-butyl)-1-phenyl- 1H-pyrazol-5-yl)ureido)-3- (methylthio)phenoxy)pyridin-2-yl)-2- hydroxyacetamide. |

77. A pharmaceutical or veterinary composition comprising a compound selected from the group consisting of

| Compound | Structure | Name |
|---|---|---|
| (a) | 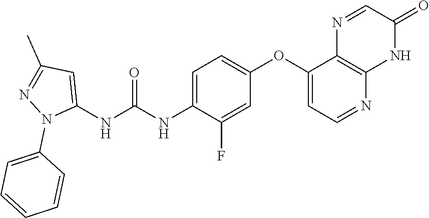 [SMILES: Cc1cc(NC(=O)Nc2ccc(Oc3ccnc4[nH]c(=O)cnc34)cc2F)n(-c2ccccc2)n1] | 1-(2-fluoro-4-((3-oxo-3,4- dihydropyrido[2,3-b]pyrazin-8- yl)oxy)phenyl)-3-(3-methyl-1- phenyl-1H-pyrazol-5-yl)urea |
| (b) | 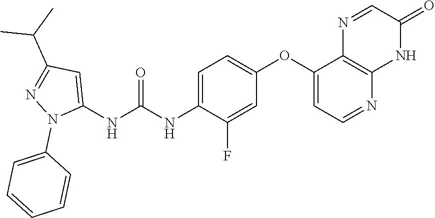 [SMILES: CC(C)c1cc(NC(=O)Nc2ccc(Oc3ccnc4[nH]c(=O)cnc34)cc2F)n(-c2ccccc2)n1] | 1-(2-fluoro-4-((3-oxo-3,4- dihydropyrido[2,3-b]pyrazin-8- yl)oxy)phenyl)-3-(3-isopropyl-1- phenyl-1H-pyrazol-5-yl)urea |
| (c) | 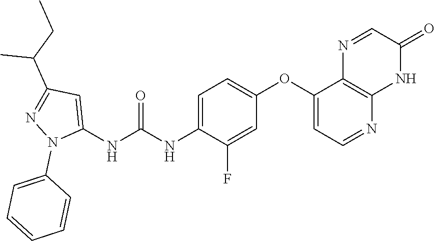 [SMILES: CCC(C)c1cc(NC(=O)Nc2ccc(Oc3ccnc4[nH]c(=O)cnc34)cc2F)n(-c2ccccc2)n1] | 1-(3-(sec-butyl)-1-phenyl-1H- pyrazol-5-yl)-3-(2-fluoro-4-((3-oxo- 3,4-dihydropyrido[2,3-b]pyrazin-8- yl)oxy)phenyl)urea |
| (d) | 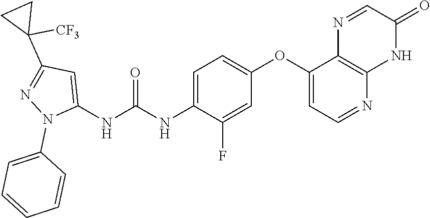 [SMILES: O=C(Nc1ccc(Oc2ccnc3[nH]c(=O)cnc23)cc1F)Nc1cc(C2(C(F)(F)F)CC2)nn1-c1ccccc1] | 1-(2-fluoro-4-((3-oxo-3,4- dihydropyrido[2,3-b]pyrazin-8- yl)oxy)phenyl)-3-(1-phenyl-3-(1- (trifluoromethyl)cyclopropyl)-1H- pyrazol-5-yl)urea |
| (e) | 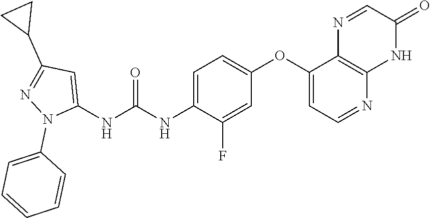 [SMILES: O=C(Nc1ccc(Oc2ccnc3[nH]c(=O)cnc23)cc1F)Nc1cc(C2CC2)nn1-c1ccccc1] | 1-(3-cyclopropyl-1-phenyl-1H- pyrazol-5-yl)-3-(2-fluoro-4-((3-oxo- 3,4-dihydropyrido[2,3-b]pyrazin-8- yl)oxy)phenyl)urea |
| (f) | 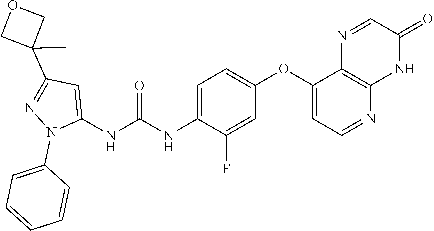 [SMILES: CC1(c2cc(NC(=O)Nc3ccc(Oc4ccnc5[nH]c(=O)cnc45)cc3F)n(-c3ccccc3)n2)COC1] | 1-(2-fluoro-4-((3-oxo-3,4- dihydropyrido[2,3-b]pyrazin-8- yl)oxy)phenyl)-3-(3-(3- methyloxetan-3-yl)-1-phenyl-1H- pyrazol-5-yl)urea |
| (g) | 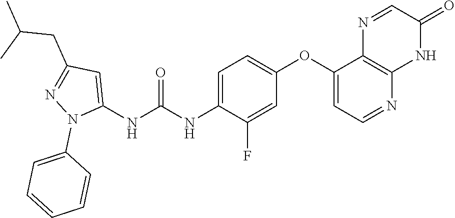 [SMILES: CC(C)Cc1cc(NC(=O)Nc2ccc(Oc3ccnc4[nH]c(=O)cnc34)cc2F)n(-c2ccccc2)n1] | 1-(2-fluoro-4-((3-oxo-3,4- dihydropyrido[2,3-b]pyrazin-8- yl)oxy)phenyl)-3-(3-isobutyl-1- phenyl-1H-pyrazol-5-yl)urea |
| (h) | 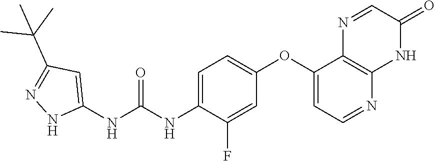 [SMILES: CC(C)(C)c1cc(NC(=O)Nc2ccc(Oc3ccnc4[nH]c(=O)cnc34)cc2F)[nH]n1] | 1-(3-(tert-butyl)-1H-pyrazol-5-yl)-3- (2-fluoro-4-((3-oxo-3,4- dihydropyrido[2,3-b]pyrazin-8- yl)oxy)phenyl)urea |
| (i) | 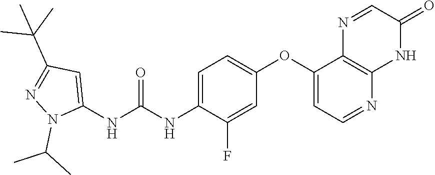 [SMILES: CC(C)n1nc(C(C)(C)C)cc1NC(=O)Nc1ccc(Oc2ccnc3[nH]c(=O)cnc23)cc1F] | 1-(3-(tert-butyl)-1-isopropyl-1H- pyrazol-5-yl)-3-(2-fluoro-4-((3-oxo- 3,4-dihydropyrido[2,3-b]pyrazin-8- yl)oxy)phenyl)urea |
| (j) | 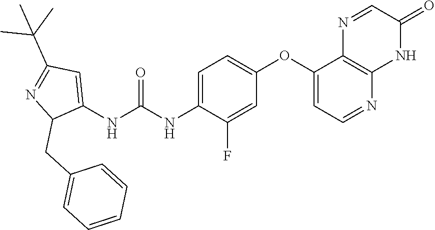 [SMILES: CC(C)(C)C1=NC(Cc2ccccc2)C(NC(=O)Nc2ccc(Oc3ccnc4[nH]c(=O)cnc34)cc2F)=C1] | 1-(1-benzyl-3-(tert-butyl)-1H- pyrazol-5-yl)-3-(2-fluoro-4-((3-oxo- 3,4-dihydropyrido[2,3-b]pyrazin-8- yl)oxy)phenyl)urea |
| (k) | 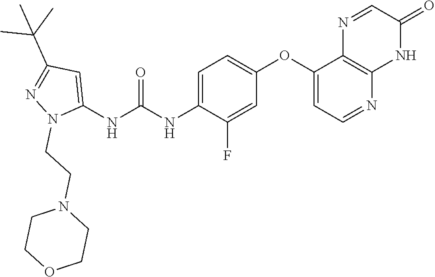 [SMILES: CC(C)(C)c1cc(NC(=O)Nc2ccc(Oc3ccnc4[nH]c(=O)cnc34)cc2F)n(CCN2CCOCC2)n1] | 1-(3-(tert-butyl)-1-(2- morpholinoethyl)-1H-pyrazol-5-yl)- 3-(2-fluoro-4-((3-oxo-3,4- dihydropyrido[2,3-b]pyrazin-8- yl)oxy)phenyl)urea |
| (l) | 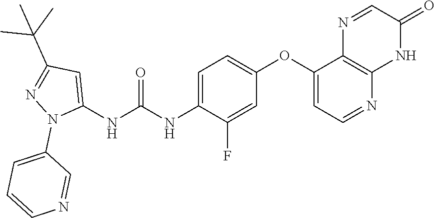 [SMILES: CC(C)(C)c1cc(NC(=O)Nc2ccc(Oc3ccnc4[nH]c(=O)cnc34)cc2F)n(-c2cccnc2)n1] | 1-(3-(tert-butyl)-1-(pyridin-3-yl)-1H- pyrazol-5-yl)-3-(2-fluoro-4-((3-oxo- 3,4-dihydropyrido[2,3-b]pyrazin-8- yl)oxy)phenyl)urea |
| (m) | 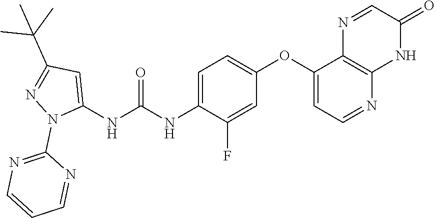 [SMILES: CC(C)(C)c1cc(NC(=O)Nc2ccc(Oc3ccnc4[nH]c(=O)cnc34)cc2F)n(-c2ncccn2)n1] | 1-(3-(tert-butyl)-1-(pyrimidin-2-yl)- 1H-pyrazol-5-yl)-3-(2-fluoro-4-((3- oxo-3,4-dihydropyrido[2,3- b]pyrazin-8-yl)oxy)phenyl)urea |
| (n) | 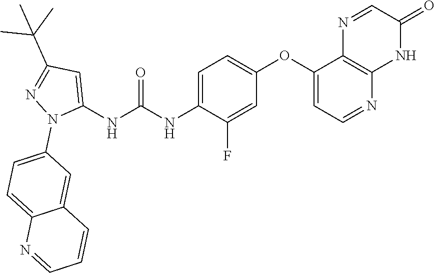 [SMILES: CC(C)(C)c1cc(NC(=O)Nc2ccc(Oc3ccnc4[nH]c(=O)cnc34)cc2F)n(-c2ccc3ncccc3c2)n1] | 1-(3-(tert-butyl)-1-(quinolin-6-yl)- 1H-pyrazol-5-yl)-3-(2-fluoro-4-((3- oxo-3,4-dihydropyrido[2,3- b]pyrazin-8-yl)oxy)phenyl)urea |
| (o) | 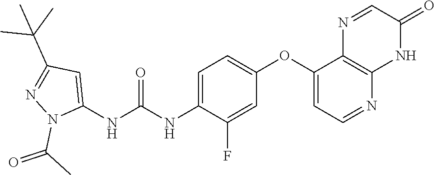 [SMILES: CC(=O)n1nc(C(C)(C)C)cc1NC(=O)Nc1ccc(Oc2ccnc3[nH]c(=O)cnc23)cc1F] | 1-(1-acetyl-3-(tert-butyl)-1H- pyrazol-5-yl)-3-(2-fluoro-4-((3-oxo- 3,4-dihydropyrido[2,3-b]pyrazin-8- yl)oxy)phenyl)urea |
| (p) | 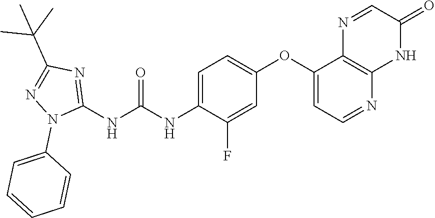 [SMILES: CC(C)(C)c1nc(NC(=O)Nc2ccc(Oc3ccnc4[nH]c(=O)cnc34)cc2F)n(-c2ccccc2)n1] | 1-(3-(tert-butyl)-1-phenyl-1H-1,2,4- triazol-5-yl)-3-(2-fluoro-4-((3-oxo- 3,4-dihydropyrido[2,3-b]pyrazin-8- yl)oxy)phenyl)urea |
| (q) | 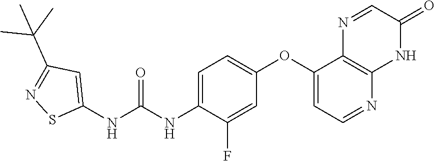 [SMILES: CC(C)(C)c1cc(NC(=O)Nc2ccc(Oc3ccnc4[nH]c(=O)cnc34)cc2F)sn1] | 1-(3-(tert-butyl)isothiazol-5-yl)-3-(2- fluoro-4-((3-oxo-3,4- dihydropyrido[2,3-b]pyrazin-8- yl)oxy)phenyl)urea |
| (r) | 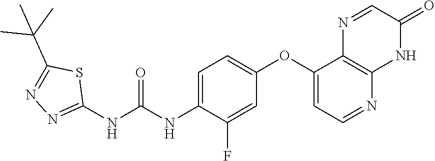 [SMILES: CC(C)(C)c1nnc(NC(=O)Nc2ccc(Oc3ccnc4[nH]c(=O)cnc34)cc2F)s1] | 1-(5-(tert-butyl)-1,3,4-thiadiazol-2- yl)-3-(2-fluoro-4-((3-oxo-3,4- dihydropyrido[2,3-b]pyrazin-8- yl)oxy)phenyl)urea |
| (s) | 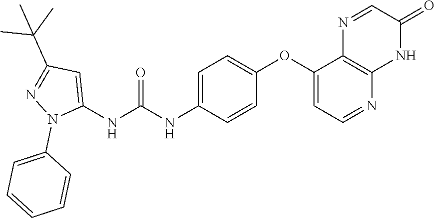 [SMILES: CC(C)(C)c1cc(NC(=O)Nc2ccc(Oc3ccnc4[nH]c(=O)cnc34)cc2)n(-c2ccccc2)n1] | 1-(3-(tert-butyl)-1-phenyl-1H- pyrazol-5-yl)-3-(4-((3-oxo-3,4- dihydropyrido[2,3-b]pyrazin-8- yl)oxy)phenyl)urea |
| (t) | 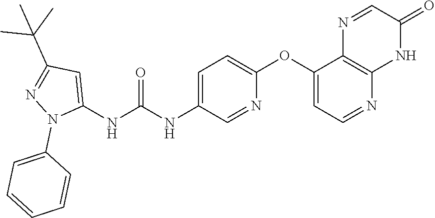 [SMILES: CC(C)(C)c1cc(NC(=O)Nc2ccc(Oc3ccnc4[nH]c(=O)cnc34)nc2)n(-c2ccccc2)n1] | 1-(3-(tert-butyl)-1-phenyl-1H- pyrazol-5-yl)-3-(6-((3-oxo-3,4- dihydropyrido[2,3-b]pyrazin-8- yl)oxy)pyridin-3-yl)urea |
| (u) | 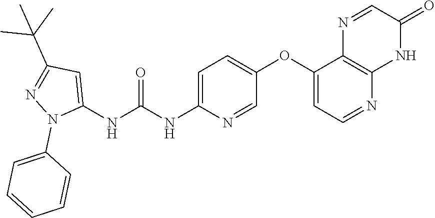 [SMILES: CC(C)(C)c1cc(NC(=O)Nc2ccc(Oc3ccnc4[nH]c(=O)cnc34)cn2)n(-c2ccccc2)n1] | 1-(3-(tert-butyl)-1-phenyl-1H- pyrazol-5-yl)-3-(5-((3-oxo-3,4- dihydropyrido[2,3-b]pyrazin-8- yl)oxy)pyridin-2-yl)urea |
| (v) | 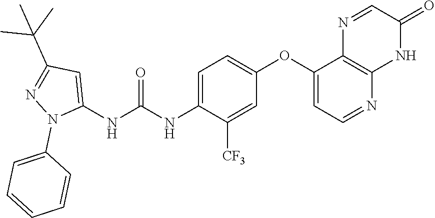 [SMILES: CC(C)(C)c1cc(NC(=O)Nc2ccc(Oc3ccnc4[nH]c(=O)cnc34)cc2C(F)(F)F)n(-c2ccccc2)n1] | 1-(3-(tert-butyl)-1-phenyl-1H- pyrazol-5-yl)-3-(4-((3-oxo-3,4- dihydropyrido[2,3-b]pyrazin-8- yl)oxy)-2- (trifluoromethyl)phenyl)urea |
| (w) | 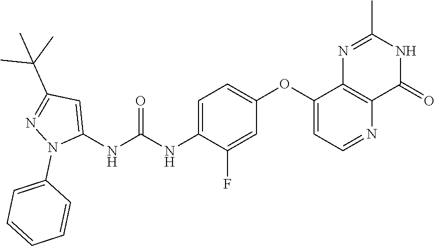 [SMILES: Cc1nc2c(Oc3ccc(NC(=O)Nc4cc(C(C)(C)C)nn4-c4ccccc4)c(F)c3)ccnc2c(=O)[nH]1] | 1-(3-(tert-butyl)-1-phenyl-1H- pyrazol-5-yl)-3-(2-fluoro-4-((2- methyl-4-oxo-3,4-dihydropyrido[3,2- d]pyrimidin-8-yl)oxy)phenyl)urea |
| (x) | 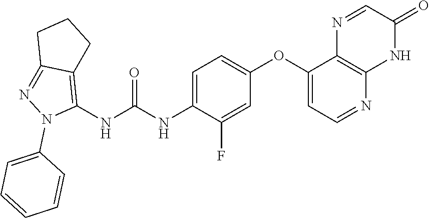 [SMILES: O=C(Nc1ccc(Oc2ccnc3[nH]c(=O)cnc23)cc1F)Nc1c2c(nn1-c1ccccc1)CCC2] | 1-(2-fluoro-4-((3-oxo-3,4- dihydropyrido[2,3-b]pyrazin-8- yl)oxy)phenyl)-3-(2-phenyl-2,4,5,6- tetrahydrocyclopenta[c]pyrazol-3- yl)urea |
| (y) | 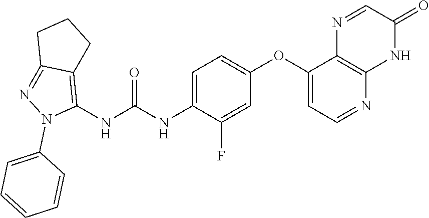 [SMILES: O=C(Nc1ccc(Oc2ccnc3[nH]c(=O)cnc23)cc1F)Nc1c2c(nn1-c1ccccc1)CCC2] | 1-(2-fluoro-4-((3-oxo-3,4- dihydropyrido[2,3-b]pyrazin-8- yl)oxy)phenyl)-3-(2-phenyl-5,6- dihydro-4H-pyrrolo[1,2-b]pyrazol-3- yl)urea |
| (z) | 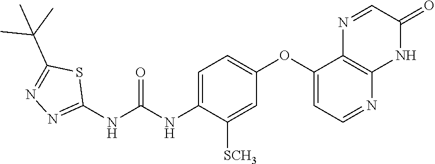 [SMILES: CSc1cc(Oc2ccnc3[nH]c(=O)cnc23)ccc1NC(=O)Nc1nnc(C(C)(C)C)s1] | 1-(5-(tert-butyl)-1,3,4-thiadiazol-2- yl)-3-(2-(methylthio)-4-((3-oxo-3,4- dihydropyrido[2,3-b]pyrazin-8- yl)oxy)phenyl)urea |
| (aa) | 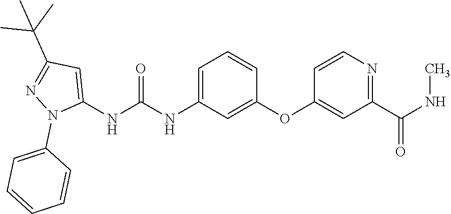 [SMILES: CNC(=O)c1cc(Oc2cccc(NC(=O)Nc3cc(C(C)(C)C)nn3-c3ccccc3)c2)ccn1] | 4-(3-(3-(3-(tert-butyl)-1-phenyl-1H- pyrazol-5-yl)ureido)phenoxy)-N- methylpicolinamide |
| (ab) | 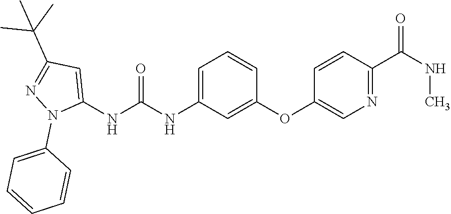 [SMILES: CNC(=O)c1ccc(Oc2cccc(NC(=O)Nc3cc(C(C)(C)C)nn3-c3ccccc3)c2)cn1] | 5-(3-(3-(3-(tert-butyl)-1-phenyl-1H- pyrazol-5-yl)ureido)phenoxy)-N- methylpicolinamide |
| (ac) | 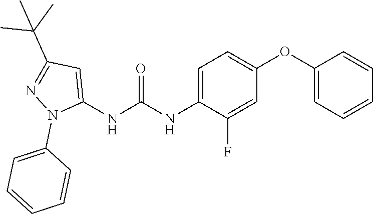 [SMILES: CC(C)(C)c1cc(NC(=O)Nc2ccc(Oc3ccccc3)cc2F)n(-c2ccccc2)n1] | 1-(3-(tert-butyl)-1-phenyl-1H- pyrazol-5-yl)-3-(2-fluoro-4- phenoxyphenyl)urea |
| (ad) | 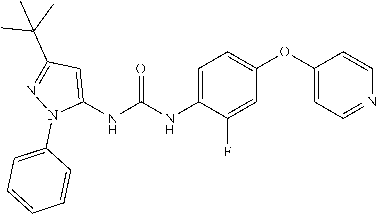 [SMILES: CC(C)(C)c1cc(NC(=O)Nc2ccc(Oc3ccncc3)cc2F)n(-c2ccccc2)n1] | 1-(3-(tert-butyl)-1-phenyl-1H- pyrazol-5-yl)-3-(2-fluoro-4-(pyridin- 4-yloxy)phenyl)urea |
| (ae) | 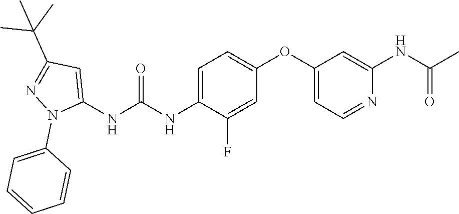 [SMILES: CC(=O)Nc1cc(Oc2ccc(NC(=O)Nc3cc(C(C)(C)C)nn3-c3ccccc3)c(F)c2)ccn1] | N-(4-(4-(3-(3-(tert-butyl)-1-phenyl- 1H-pyrazol-5-yl)ureido)-3- fluorophenoxy)pyridin-2- yl)acetamide |
| (af) | 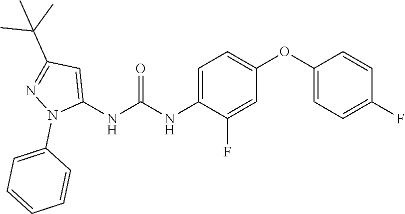 [SMILES: CC(C)(C)c1cc(NC(=O)Nc2ccc(Oc3ccc(F)cc3)cc2F)n(-c2ccccc2)n1] | 1-(3-(tert-butyl)-1-phenyl-1H- pyrazol-5-yl)-3-(2-fluoro-4-(4- fluorophenoxy)phenyl)urea |
| (ag) | 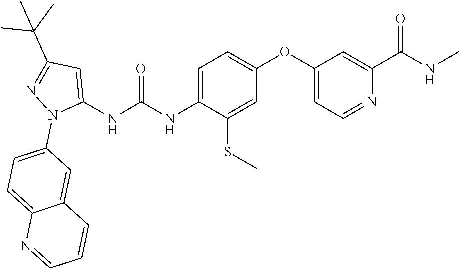 [SMILES: CNC(=O)c1cc(Oc2ccc(NC(=O)Nc3cc(C(C)(C)C)nn3-c3ccc4ncccc4c3)c(SC)c2)ccn1] | 4-(4-(3-(3-(tert-butyl)-1-(quinolin-6- yl)-1H-pyrazol-5-yl)ureido)-3- (methylthio)phenoxy)-N- methylpicolinamide |
| (ah) | 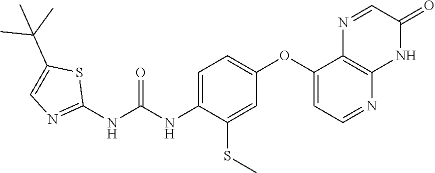 [SMILES: CSc1cc(Oc2ccnc3[nH]c(=O)cnc23)ccc1NC(=O)Nc1ncc(C(C)(C)C)s1] | 1-(5-(tert-butyl)thiazol-2-yl)-3-(2- (methylthio)-4-((3-oxo-3,4- dihydropyrido[2,3-b]pyrazin-8- yl)oxy)phenyl)urea |
| (ai) | 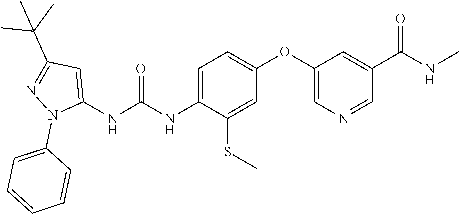 [SMILES: CNC(=O)c1cncc(Oc2ccc(NC(=O)Nc3cc(C(C)(C)C)nn3-c3ccccc3)c(SC)c2)c1] | 5-(4-(3-(3-(tert-butyl)-1-phenyl-1H- pyrazol-5-yl)ureido)-3- (methylthio)phenoxy)-N- methylnicotinamide |
| (aj) | 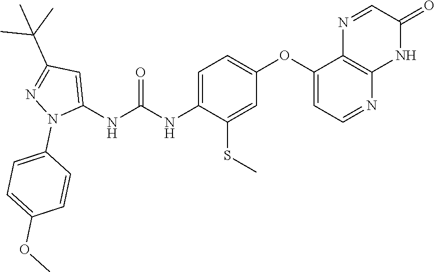 [SMILES: COc1ccc(-n2nc(C(C)(C)C)cc2NC(=O)Nc2ccc(Oc3ccnc4[nH]c(=O)cnc34)cc2SC)cc1] | 1-(3-(tert-butyl)-1-(4- methoxyphenyl)-1H-pyrazol-5-yl)-3- (2-(methylthio)-4-((3-oxo-3,4- dihydropyrido[2,3-b]pyrazin-8- yl)oxy)phenyl)urea |
| (ak) | 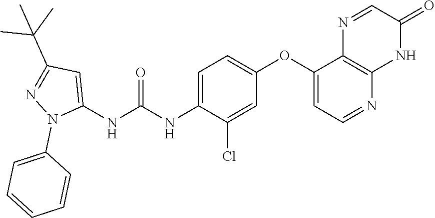 [SMILES: CC(C)(C)c1cc(NC(=O)Nc2ccc(Oc3ccnc4[nH]c(=O)cnc34)cc2Cl)n(-c2ccccc2)n1] | 1-(3-(tert-butyl)-1-phenyl-1H- pyrazol-5-yl)-3-(2-chloro-4-((3-oxo- 3,4-dihydropyrido[2,3-b]pyrazin-8- yl)oxy)phenyl)urea |
| (al) | 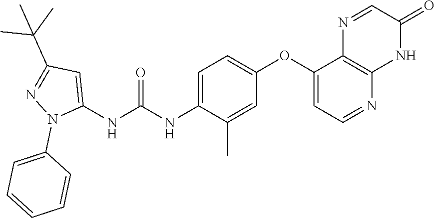 [SMILES: Cc1cc(Oc2ccnc3[nH]c(=O)cnc23)ccc1NC(=O)Nc1cc(C(C)(C)C)nn1-c1ccccc1] | 1-(3-(tert-butyl)-1-phenyl-1H- pyrazol-5-yl)-3-(2-methyl-4-((3-oxo- 3,4-dihydropyrido[2,3-b]pyrazin-8- yl)oxy)phenyl)urea |
| (am) | 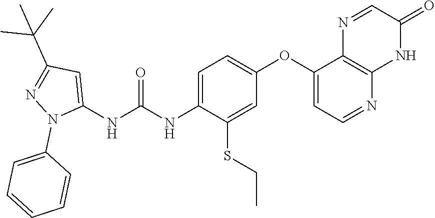 [SMILES: CCSc1cc(Oc2ccnc3[nH]c(=O)cnc23)ccc1NC(=O)Nc1cc(C(C)(C)C)nn1-c1ccccc1] | 1-(3-(tert-butyl)-1-phenyl-1H- pyrazol-5-yl)-3-(2-(ethylthio)-4-((3- oxo-3,4-dihydropyrido[2,3- b]pyrazin-8-yl)oxy)phenyl)urea |
| (an) | 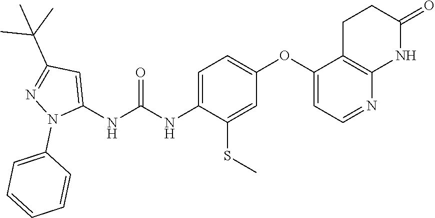 [SMILES: CSc1cc(Oc2ccnc3c2CCC(=O)N3)ccc1NC(=O)Nc1cc(C(C)(C)C)nn1-c1ccccc1] | 1-(3-(tert-butyl)-1-phenyl-1H- pyrazol-5-yl)-3-(2-(methylthio)-4- ((7-oxo-5,6,7,8-tetrahydro-1,8- naphthyridin-4-yl)oxy)phenyl)urea |
| (ao) | 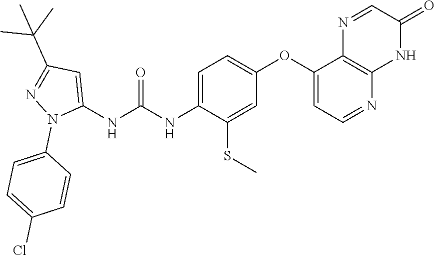 [SMILES: CSc1cc(Oc2ccnc3[nH]c(=O)cnc23)ccc1NC(=O)Nc1cc(C(C)(C)C)nn1-c1ccc(Cl)cc1] | 1-(3-(tert-butyl)-1-(4-chlorophenyl)- 1H-pyrazol-5-yl)-3-(2-(methylthio)- 4-((3-oxo-3,4-dihydropyrido[2,3- b]pyrazin-8-yl)oxy)phenyl)urea |
| (ap) | 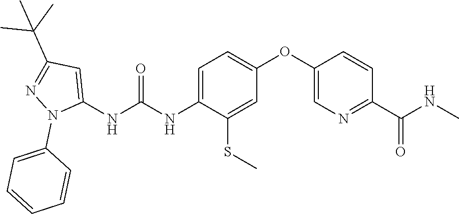 [SMILES: CNC(=O)c1ccc(Oc2ccc(NC(=O)Nc3cc(C(C)(C)C)nn3-c3ccccc3)c(SC)c2)cn1] | 5-(4-(3-(3-(tert-butyl)-1-phenyl-1H- pyrazol-5-yl)ureido)-3- (methylthio)phenoxy)-N- methylpicolinamide |
| (aq) | 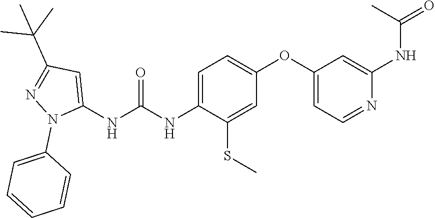 [SMILES: CSc1cc(Oc2ccnc(NC(C)=O)c2)ccc1NC(=O)Nc1cc(C(C)(C)C)nn1-c1ccccc1] | N-(4-(4-(3-(3-(tert-butyl)-1-phenyl- 1H-pyrazol-5-yl)ureido)-3- (methylthio)phenoxy)pyridin-2- yl)acetamide |
| (ar) | 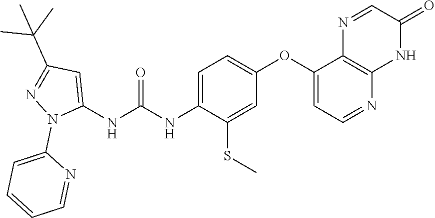 [SMILES: CSc1cc(Oc2ccnc3[nH]c(=O)cnc23)ccc1NC(=O)Nc1cc(C(C)(C)C)nn1-c1ccccn1] | 1-(3-(tert-butyl)-1-(pyridin-2-yl)-1H- pyrazol-5-yl)-3-(2-(methylthio)-4- ((3-oxo-3,4-dihydropyrido[2,3- b]pyrazin-8-yl)oxy)phenyl)urea |
| (as) | 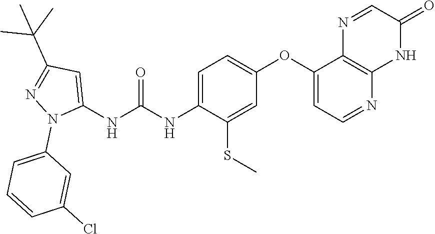 [SMILES: CSc1cc(Oc2ccnc3[nH]c(=O)cnc23)ccc1NC(=O)Nc1cc(C(C)(C)C)nn1-c1cccc(Cl)c1] | 1-(3-(tert-butyl)-1-(3-chlorophenyl)- 1H-pyrazol-5-yl)-3-(2-(methylthio)- 4-((3-oxo-3,4-dihydropyrido[2,3- b]pyrazin-8-yl)oxy)phenyl)urea |
| (at) | 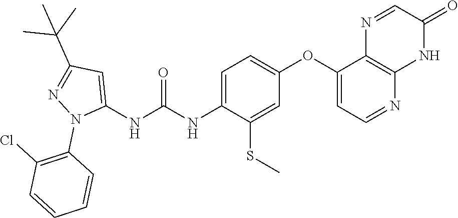 [SMILES: CSc1cc(Oc2ccnc3[nH]c(=O)cnc23)ccc1NC(=O)Nc1cc(C(C)(C)C)nn1-c1ccccc1Cl] | 1-(3-(tert-butyl)-1-(2-chlorophenyl)- 1H-pyrazol-5-yl)-3-(2-(methylthio)- 4-((3-oxo-3,4-dihydropyrido[2,3- b]pyrazin-8-yl)oxy)phenyl)urea |
| (au) | 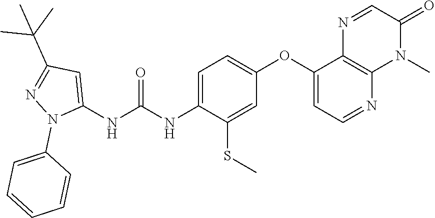 [SMILES: CSc1cc(Oc2ccnc3c2ncc(=O)n3C)ccc1NC(=O)Nc1cc(C(C)(C)C)nn1-c1ccccc1] | 1-(3-(tert-butyl)-1-phenyl-1H- pyrazol-5-yl)-3-(4-((4-methyl-3-oxo- 3,4-dihydropyrido[2,3-b]pyrazin-8- yl)oxy)-2-(methylthio)phenyl)urea |
| (av) | 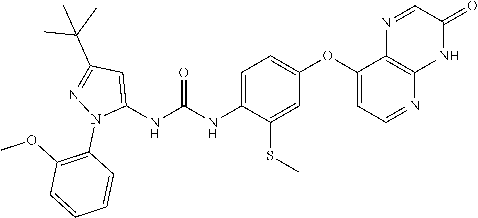 [SMILES: COc1ccccc1-n1nc(C(C)(C)C)cc1NC(=O)Nc1ccc(Oc2ccnc3[nH]c(=O)cnc23)cc1SC] | 1-(3-(tert-butyl)-1-(2- methoxyphenyl)-1H-pyrazol-5-yl)-3- (2-(methylthio)-4-((3-oxo-3,4- dihydropyrido[2,3-b]pyrazin-8- yl)oxy)phenyl)urea |
| (aw) | 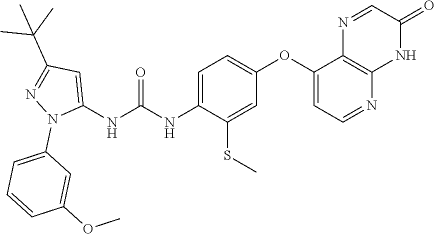 [SMILES: COc1cccc(-n2nc(C(C)(C)C)cc2NC(=O)Nc2ccc(Oc3ccnc4[nH]c(=O)cnc34)cc2SC)c1] | 1-(3-(tert-butyl)-1-(3- methoxyphenyl)-1H-pyrazol-5-yl)-3- (2-(methylthio)-4-((3-oxo-3,4- dihydropyrido[2,3-b]pyrazin-8- yl)oxy)phenyl)urea |
| (ax) | 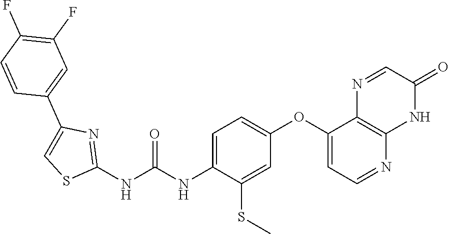 [SMILES: CSc1cc(Oc2ccnc3[nH]c(=O)cnc23)ccc1NC(=O)Nc1nc(-c2ccc(F)c(F)c2)cs1] | 1-(4-(3,4-difluorophenyl)thiazol-2- yl)-3-(2-(methylthio)-4-((3-oxo-3,4- dihydropyrido[2,3-b]pyrazin-8- yl)oxy)phenyl)urea |
| (ay) | 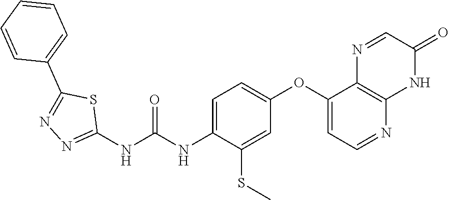 [SMILES: CSc1cc(Oc2ccnc3[nH]c(=O)cnc23)ccc1NC(=O)Nc1nnc(-c2ccccc2)s1] | 1-(2-(methylthio)-4-((3-oxo-3,4- dihydropyrido[2,3-b]pyrazin-8- yl)oxy)phenyl)-3-(5-phenyl-1,3,4- thiadiazol-2-yl)urea |
| (az) | 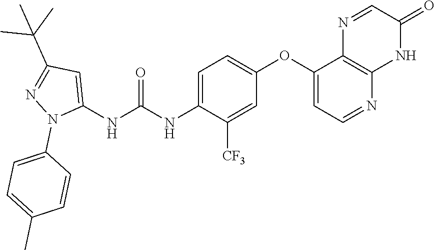 [SMILES: Cc1ccc(-n2nc(C(C)(C)C)cc2NC(=O)Nc2ccc(Oc3ccnc4[nH]c(=O)cnc34)cc2C(F)(F)F)cc1] | 1-(3-(tert-butyl)-1-(p-tolyl)-1H- pyrazol-5-yl)-3-(4-((3-oxo-3,4- dihydropyrido[2,3-b]pyrazin-8- yl)oxy)-2- (trifluoromethyl)phenyl)urea |
| (ba) | 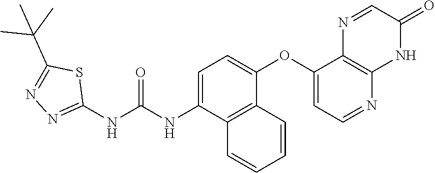 [SMILES: CC(C)(C)c1nnc(NC(=O)Nc2ccc(Oc3ccnc4[nH]c(=O)cnc34)c3ccccc23)s1] | 1-(5-(tert-butyl)thiazol-2-yl)-3-(4- ((3-oxo-3,4-dihydropyrido[2,3- b]pyrazin-8-yl)oxy)naphthalen-1- yl)urea |
| (bb) | 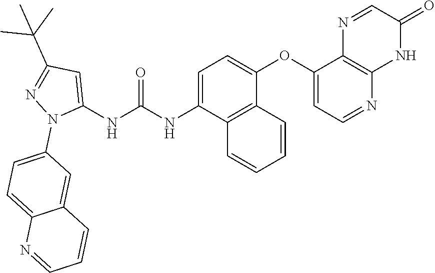 [SMILES: CC(C)(C)c1cc(NC(=O)Nc2ccc(Oc3ccnc4[nH]c(=O)cnc34)c3ccccc23)n(-c2ccc3ncccc3c2)n1] | 1-(3-(tert-butyl)-1-(quinolin-6-yl)- 1H-pyrazol-5-yl)-3-(4-((3-oxo-3,4- dihydropyrido[2,3-b]pyrazin-8- yl)oxy)naphthalen-1-yl)urea |
| (bc) | 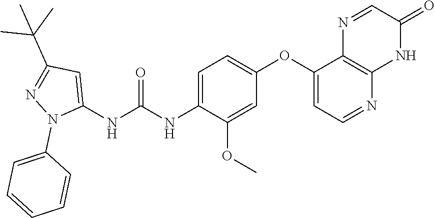 [SMILES: COc1cc(Oc2ccnc3[nH]c(=O)cnc23)ccc1NC(=O)Nc1cc(C(C)(C)C)nn1-c1ccccc1] | 1-(3-(tert-butyl)-1-phenyl-1H- pyrazol-5-yl)-3-(2-methoxy-4-((3- oxo-3,4-dihydropyrido[2,3- b]pyrazin-8-yl)oxy)phenyl)urea |
| (bd) | 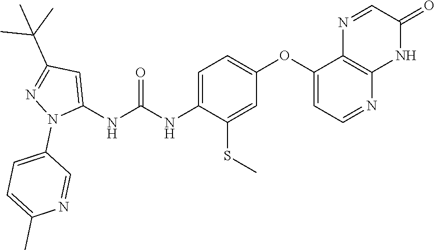 [SMILES: CSc1cc(Oc2ccnc3[nH]c(=O)cnc23)ccc1NC(=O)Nc1cc(C(C)(C)C)nn1-c1ccc(C)nc1] | 1-(3-(tert-butyl)-1-(6-methylpyridin- 3-yl)-1H-pyrazol-5-yl)-3-(2- (methylthio)-4-((3-oxo-3,4- dihydropyrido[2,3-b]pyrazin-8- yl)oxy)phenyl)urea |
| (be) | 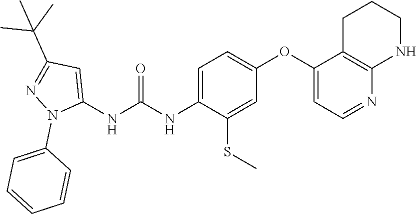 [SMILES: CSc1cc(Oc2ccnc3c2CCCN3)ccc1NC(=O)Nc1cc(C(C)(C)C)nn1-c1ccccc1] | 1-(3-(tert-butyl)-1-phenyl-1H- pyrazol-5-yl)-3-(2-(methylthio)-4- ((5,6,7,8-tetrahydro-1,8- naphthyridin-4-yl)oxy)phenyl)urea |
| (bf) | 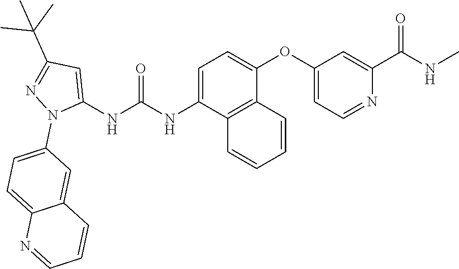 [SMILES: CNC(=O)c1cc(Oc2ccc(NC(=O)Nc3cc(C(C)(C)C)nn3-c3ccc4ncccc4c3)c3ccccc23)ccn1] | 4-((4-(3-(3-(tert-butyl)-1-(quinolin-6- yl)-1H-pyrazol-5- yl)ureido)naphthalen-1-yl)oxy)-N- methylpicolinamide |
| (bg) | 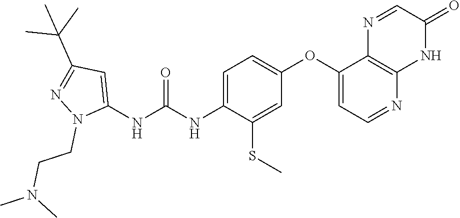 [SMILES: CSc1cc(Oc2ccnc3[nH]c(=O)cnc23)ccc1NC(=O)Nc1cc(C(C)(C)C)nn1CCN(C)C] | 1-(3-(tert-butyl)-1-(2- (dimethylamino)ethyl)-1H-pyrazol-5- yl)-3-(2-(methylthio)-4-((3-oxo-3,4- dihydropyrido[2,3-b]pyrazin-8- yl)oxy)phenyl)urea |
| (bh) | 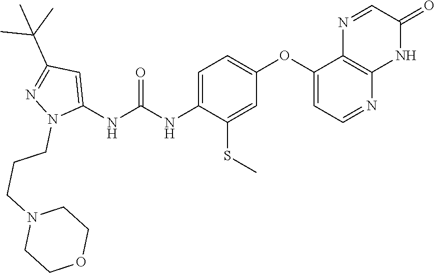 [SMILES: CSc1cc(Oc2ccnc3[nH]c(=O)cnc23)ccc1NC(=O)Nc1cc(C(C)(C)C)nn1CCCN1CCOCC1] | 1-(3-(tert-butyl)-1-(3- morpholinopropyl)-1H-pyrazol-5-yl)- 3-(2-(methylthio)-4-((3-oxo-3,4- dihydropyrido[2,3-b]pyrazin-8- yl)oxy)phenyl)urea |
| (bi) | 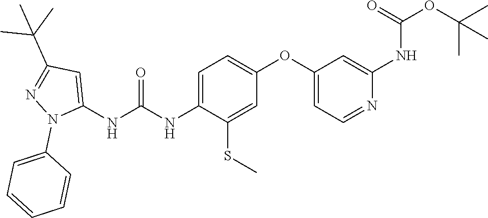 [SMILES: CSc1cc(Oc2ccnc(NC(=O)OC(C)(C)C)c2)ccc1NC(=O)Nc1cc(C(C)(C)C)nn1-c1ccccc1] | tert-butyl (4-(4-(3-(3-(tert-butyl)- phenyl-1H-pyrazol-5-yl)ureido)-3- (methylthio)phenoxy)pyridin-2- yl)carbamate |
| (bj) | 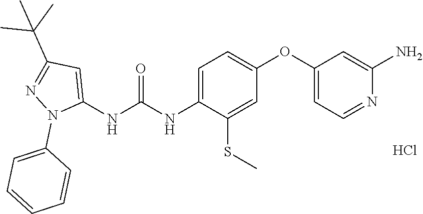 [SMILES: CSc1cc(Oc2ccnc(N)c2)ccc1NC(=O)Nc1cc(C(C)(C)C)nn1-c1ccccc1 ; Cl] | 1-(4-((2-aminopyridin-4-yl)oxy)-2- (methylthio)phenyl)-3-(3-(tert-butyl)- 1-phenyl-1H-pyrazol-5-yl)urea hydrochloride |
| (bk) | 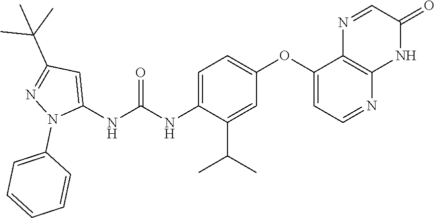 [SMILES: CC(C)c1cc(Oc2ccnc3[nH]c(=O)cnc23)ccc1NC(=O)Nc1cc(C(C)(C)C)nn1-c1ccccc1] | 1-(3-(tert-butyl)-1-phenyl-1H- pyrazol-5-yl)-3-(2-isopropyl-4-((3- oxo-3,4-dihydropyrido[2,3- b]pyrazin-8-yl)oxy)phenyl)urea |
| (bl) | 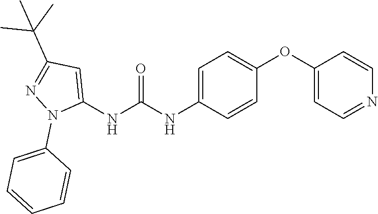 [SMILES: CC(C)(C)c1cc(NC(=O)Nc2ccc(Oc3ccncc3)cc2)n(-c2ccccc2)n1] | 1-(3-(tert-butyl)-1-phenyl-1H- pyrazol-5-yl)-3-(4-(pyridin-4- yloxy)phenyl)urea |
| (bm) | 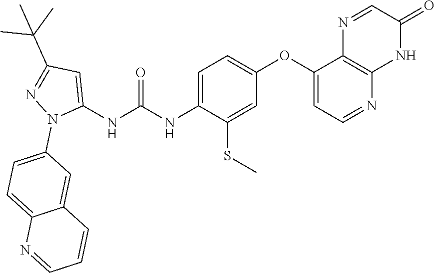 [SMILES: CSc1cc(Oc2ccnc3[nH]c(=O)cnc23)ccc1NC(=O)Nc1cc(C(C)(C)C)nn1-c1ccc2ncccc2c1] | 1-(3-(tert-butyl)-1-(quinolin-6-yl)- 1H-pyrazol-5-yl)-3-(2-(methylthio)- 4-((3-oxo-3,4-dihydropyrido[2,3- b]pyrazin-8-yl)oxy)phenyl)urea |
| (bn) | 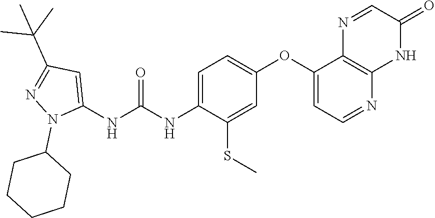 [SMILES: CSc1cc(Oc2ccnc3[nH]c(=O)cnc23)ccc1NC(=O)Nc1cc(C(C)(C)C)nn1C1CCCCC1] | 1-(3-(tert-butyl)-1-cyclohexyl-1H- pyrazol-5-yl)-3-(2-(methylthio)-4- ((3-oxo-3,4-dihydropyrido[2,3- b]pyrazin-8-yl)oxy)phenyl)urea |
| (bo) | 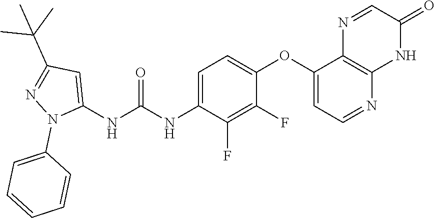 [SMILES: CC(C)(C)c1cc(NC(=O)Nc2ccc(Oc3ccnc4[nH]c(=O)cnc34)c(F)c2F)n(-c2ccccc2)n1] | 1-(3-(tert-butyl)-1-phenyl-1H- pyrazol-5-yl)-3-(2,3-difluoro-4-((3- oxo-3,4-dihydropyrido[2,3- b]pyrazin-8-yl)oxy)phenyl)urea |
| (bp) | 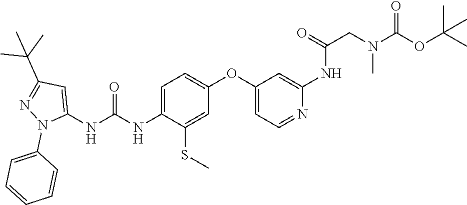 [SMILES: CSc1cc(Oc2ccnc(NC(=O)CN(C)C(=O)OC(C)(C)C)c2)ccc1NC(=O)Nc1cc(C(C)(C)C)nn1-c1ccccc1] | tert-butyl (2-((4-(4-(3-(3-(tert-butyl)- 1-phenyl-1H-pyrazol-5-yl)ureido)-3- (methylthio)phenoxy)pyridin-2- yl)amino)-2- oxoethyl)(methyl)carbamate |
| (bq) | 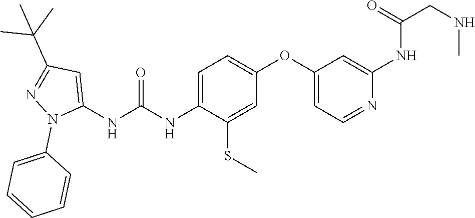 [SMILES: CNCC(=O)Nc1cc(Oc2ccc(NC(=O)Nc3cc(C(C)(C)C)nn3-c3ccccc3)c(SC)c2)ccn1] | N-(4-(4-(3-(3-(tert-butyl)-1-phenyl- 1H-pyrazol-5-yl)ureido)-3- (methylthio)phenoxy)pyridin-2-yl)-2- (methylamino)acetamide |
| (br) | 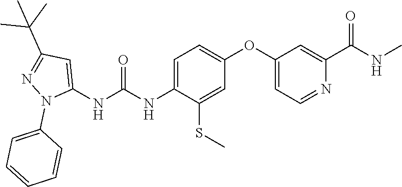 [SMILES: CNC(=O)c1cc(Oc2ccc(NC(=O)Nc3cc(C(C)(C)C)nn3-c3ccccc3)c(SC)c2)ccn1] | 4-(4-(3-(3-(tert-butyl)-1-phenyl-1H- pyrazol-5-yl)ureido)-3- (methylthio)phenoxy)-N- methylpicolinamide |
| (bs) | 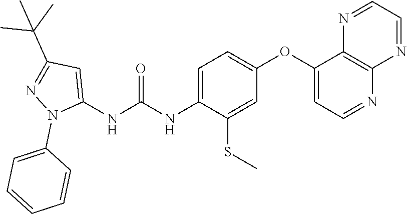 [SMILES: CSc1cc(Oc2ccnc3nccnc23)ccc1NC(=O)Nc1cc(C(C)(C)C)nn1-c1ccccc1] | 1-(3-(tert-butyl)-1-phenyl-1H- pyrazol-5-yl)-3-(2-(methylthio)-4- (pyrido[2,3-b]pyrazin-8- yloxy)phenyl)urea |
| (bt) | 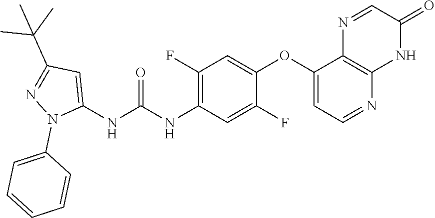 [SMILES: CC(C)(C)c1cc(NC(=O)Nc2cc(F)c(Oc3ccnc4[nH]c(=O)cnc34)cc2F)n(-c2ccccc2)n1] | 1-(3-(tert-butyl)-1-phenyl-1H- pyrazol-5-yl)-3-(2,5-difluoro-4-((3- oxo-3,4-dihydropyrido[2,3- b]pyrazin-8-yl)oxy)phenyl)urea |
| (bu) | 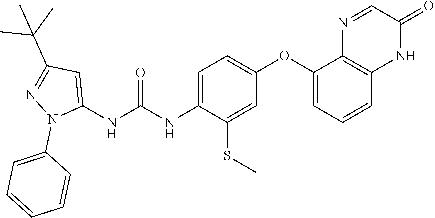 [SMILES: CSc1cc(Oc2cccc3[nH]c(=O)cnc23)ccc1NC(=O)Nc1cc(C(C)(C)C)nn1-c1ccccc1] | 1-(3-(tert-butyl)-1-phenyl-1H- pyrazol-5-yl)-3-(2-(methylthio)-4- ((2-oxo-1,2-dihydroquinoxalin-5- yl)oxy)phenyl)urea |
| (bv) | 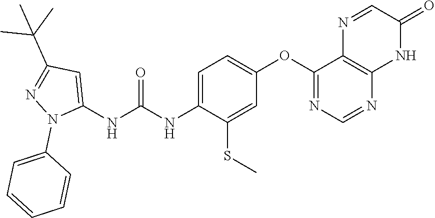 [SMILES: CSc1cc(Oc2ncnc3[nH]c(=O)cnc23)ccc1NC(=O)Nc1cc(C(C)(C)C)nn1-c1ccccc1] | 1-(3-(tert-butyl)-1-phenyl-1H- pyrazol-5-yl)-3-(2-(methylthio)-4- ((7-oxo-7,8-dihydropteridin-4- yl)oxy)phenyl)urea |
| (bw) | 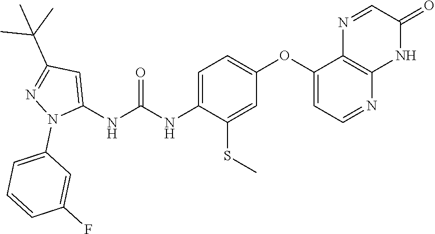 [SMILES: CSc1cc(Oc2ccnc3[nH]c(=O)cnc23)ccc1NC(=O)Nc1cc(C(C)(C)C)nn1-c1cccc(F)c1] | 1-(3-(tert-butyl)-1-(3-fluorophenyl)- 1H-pyrazol-5-yl)-3-(2-(methylthio)- 4-((3-oxo-3,4-dihydropyrido[2,3- b]pyrazin-8-yl)oxy)phenyl)urea |
| (bx) | 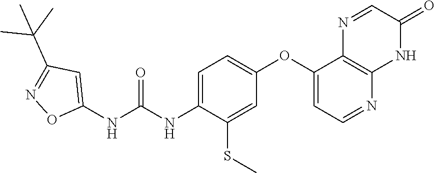 [SMILES: CSc1cc(Oc2ccnc3[nH]c(=O)cnc23)ccc1NC(=O)Nc1cc(C(C)(C)C)no1] | 1-(3-(tert-butyl)isoxazol-5-yl)-3-(2- (methylthio)-4-((3-oxo-3,4- dihydropyrido[2,3-b]pyrazin-8- yl)oxy)phenyl)urea |
| (by) | 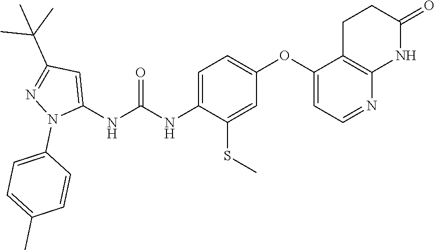 [SMILES: CSc1cc(Oc2ccnc3c2CCC(=O)N3)ccc1NC(=O)Nc1cc(C(C)(C)C)nn1-c1ccc(C)cc1] | 1-(3-(tert-butyl)-1-(p-tolyl)-1H- pyrazol-5-yl)-3-(2-(methylthio)-4- ((7-oxo-5,6,7,8-tetrahydro-1,8- naphthyridin-4-yl)oxy)phenyl)urea |
| (bz) | 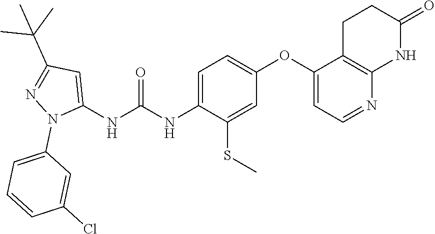 [SMILES: CSc1cc(Oc2ccnc3c2CCC(=O)N3)ccc1NC(=O)Nc1cc(C(C)(C)C)nn1-c1cccc(Cl)c1] | 1-(3-(tert-butyl)-1-(3-chlorophenyl)- 1H-pyrazol-5-yl)-3-(2-(methylthio)- 4-((7-oxo-5,6,7,8-tetrahydro-1,8- naphthyridin-4-yl)oxy)phenyl)urea |
| (ca) | 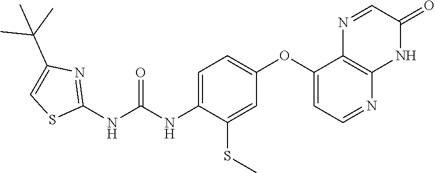 [SMILES: CSc1cc(Oc2ccnc3[nH]c(=O)cnc23)ccc1NC(=O)Nc1nc(C(C)(C)C)cs1] | 1-(4-(tert-butyl)thiazol-2-yl)-3-(2- (methylthio)-4-((3-oxo-3,4- dihydropyrido[2,3-b]pyrazin-8- yl)oxy)phenyl)urea |
| (cb) | 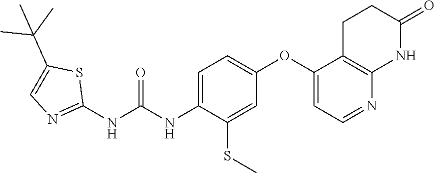 [SMILES: CSc1cc(Oc2ccnc3c2CCC(=O)N3)ccc1NC(=O)Nc1ncc(C(C)(C)C)s1] | 1-(5-(tert-butyl)thiazol-2-yl)-3-(2- (methylthio)-4-((7-oxo-5,6,7,8- tetrahydro-1,8-naphthyridin-4- yl)oxy)phenyl)urea |
| (cc) | 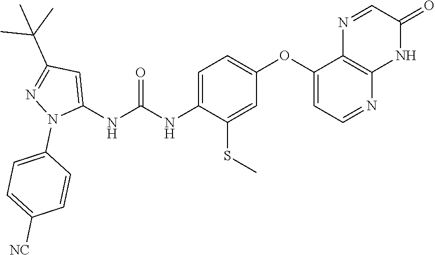 [SMILES: CSc1cc(Oc2ccnc3[nH]c(=O)cnc23)ccc1NC(=O)Nc1cc(C(C)(C)C)nn1-c1ccc(C#N)cc1] | 1-(3-(tert-butyl)-1-(4-cyanophenyl)- 1H-pyrazol-5-yl)-3-(2-(methylthio)- 4-((3-oxo-3,4-dihydropyrido[2,3- b]pyrazin-8-yl)oxy)phenyl)urea |
| (cd) | 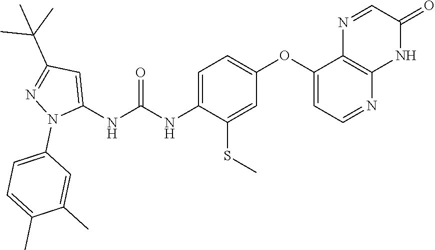 [SMILES: CSc1cc(Oc2ccnc3[nH]c(=O)cnc23)ccc1NC(=O)Nc1cc(C(C)(C)C)nn1-c1ccc(C)c(C)c1] | 1-(3-(tert-butyl)-1-(3,4- dimethylphenyl)-1H-pyrazol-5-yl)-3- (2-(methylthio)-4-((3-oxo-3,4- dihydropyrido[2,3-b]pyrazin-8- yl)oxy)phenyl)urea |
| (ce) | 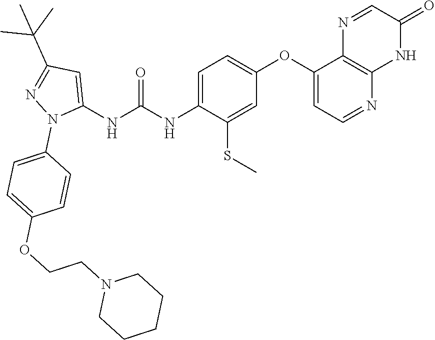 [SMILES: CSc1cc(Oc2ccnc3[nH]c(=O)cnc23)ccc1NC(=O)Nc1cc(C(C)(C)C)nn1-c1ccc(OCCN2CCCCC2)cc1] | 1-(3-(tert-butyl)-1-(4-(2-(piperidin-1- yl)ethoxy)phenyl)-1H-pyrazol-5-yl)- 3-(2-(methylthio)-4-((3-oxo-3,4- dihydropyrido[2,3-b]pyrazin-8- yl)oxy)phenyl)urea |
| (cf) | 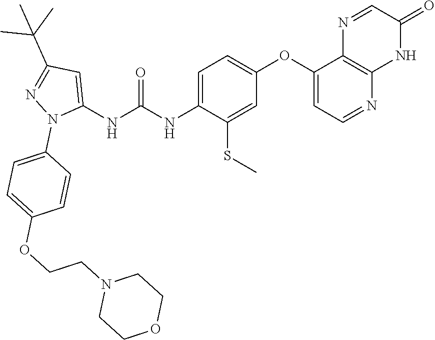 [SMILES: CSc1cc(Oc2ccnc3[nH]c(=O)cnc23)ccc1NC(=O)Nc1cc(C(C)(C)C)nn1-c1ccc(OCCN2CCOCC2)cc1] | 1-(3-(tert-butyl)-1-(4-(2- morpholinoethoxy)phenyl)-1H- pyrazol-5-yl)-3-(2-(methylthio)-4- ((3-oxo-3,4-dihydropyrido[2,3- b]pyrazin-8-yl)oxy)phenyl)urea |
| (cg) | 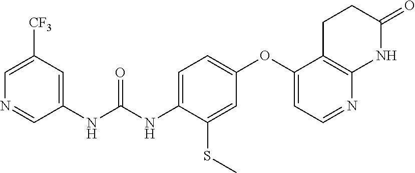 [SMILES: CSc1cc(Oc2ccnc3c2CCC(=O)N3)ccc1NC(=O)Nc1cncc(C(F)(F)F)c1] | 1-(2-(methylthio)-4-((7-oxo-5,6,7,8- tetrahydro-1,8-naphthyridin-4- yl)oxy)phenyl)-3-(5- (trifluoromethyl)pyridin-3-yl)urea |
| (ch) | 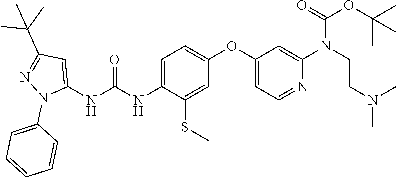 [SMILES: CSc1cc(Oc2ccnc(N(CCN(C)C)C(=O)OC(C)(C)C)c2)ccc1NC(=O)Nc1cc(C(C)(C)C)nn1-c1ccccc1] | tert-butyl (4-(4-(3-(3-(tert-butyl)-1- phenyl-1H-pyrazol-5-yl)ureido)-3- (methylthio)phenoxy)pyridin-2-yl)(2- (dimethylamino)ethyl)carbamate |
| (ci) | 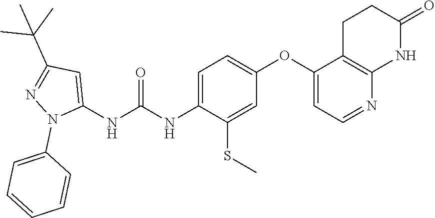 [SMILES: CSc1cc(Oc2ccnc3c2CCC(=O)N3)ccc1NC(=O)Nc1cc(C(C)(C)C)nn1-c1ccccc1] | 1-(3-(tert-butyl)-1-phenyl-1H- pyrazol-5-yl)-3-(2-(methylthio)-4- ((2-oxo-1,2,3,4-tetrahydroquinolin-5- yl)oxy)phenyl)urea |
| (cj) | 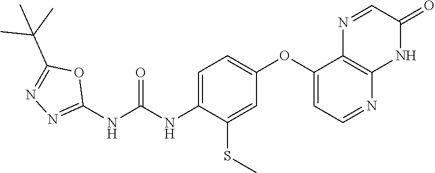 [SMILES: CSc1cc(Oc2ccnc3[nH]c(=O)cnc23)ccc1NC(=O)Nc1nnc(C(C)(C)C)o1] | 1-(5-(tert-butyl)-1,3,4-oxadiazol-2- yl)-3-(2-(methylthio)-4-((3-oxo-3,4- dihydropyrido[2,3-b]pyrazin-8- yl)oxy)phenyl)urea |
| (ck) | 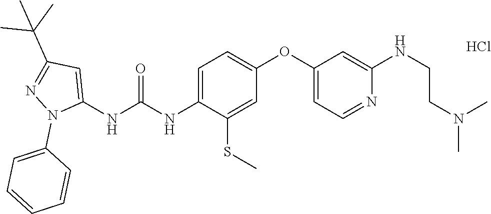 [SMILES: CSc1cc(Oc2ccnc(NCCN(C)C)c2)ccc1NC(=O)Nc1cc(C(C)(C)C)nn1-c1ccccc1 ; Cl] | 1-(3-(tert-butyl)-1-phenyl-1H- pyrazol-5-yl)-3-(4-((2-((2- (dimethylamino)ethyl)amino)pyridin- 4-yl)oxy)-2-(methylthio)phenyl)urea hydrochloride |
| (cl) | 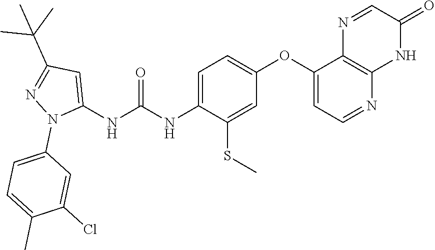 [SMILES: CSc1cc(Oc2ccnc3[nH]c(=O)cnc23)ccc1NC(=O)Nc1cc(C(C)(C)C)nn1-c1ccc(C)c(Cl)c1] | 1-(3-(tert-butyl)-1-(3-chloro-4- methylphenyl)-1H-pyrazol-5-yl)-3- (2-(methylthio)-4-((3-oxo-3,4- dihydropyrido[2,3-b]pyrazin-8- yl)oxy)phenyl)urea |
| (cm) | 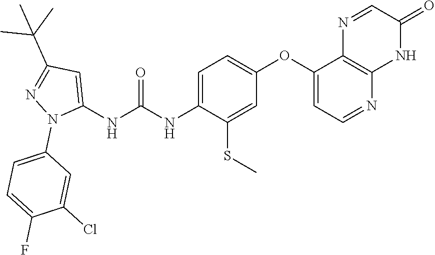 [SMILES: CSc1cc(Oc2ccnc3[nH]c(=O)cnc23)ccc1NC(=O)Nc1cc(C(C)(C)C)nn1-c1ccc(F)c(Cl)c1] | 1-(3-(tert-butyl)-1-(3-chloro-4- fluorophenyl)-1H-pyrazol-5-yl)-3-(2- (methylthio)-4-((3-oxo-3,4- dihydropyrido[2,3-b]pyrazin-8- yl)oxy)phenyl)urea |
| (cn) | 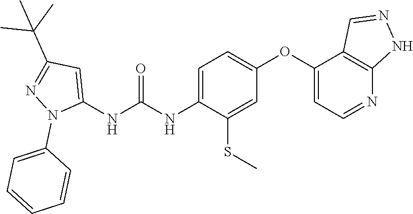 [SMILES: CSc1cc(Oc2ccnc3[nH]ncc23)ccc1NC(=O)Nc1cc(C(C)(C)C)nn1-c1ccccc1] | 1-(4-((1H-indazol-4-yl)oxy)-2- (methylthio)phenyl)-3-(3-(tert-butyl)- 1-phenyl-1H-pyrazol-5-yl)urea |
| (co) | 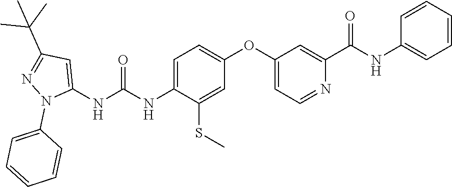 [SMILES: CSc1cc(Oc2ccnc(C(=O)Nc3ccccc3)c2)ccc1NC(=O)Nc1cc(C(C)(C)C)nn1-c1ccccc1] | 4-(4-(3-(3-(tert-butyl)-1-phenyl-1H- pyrazol-5-yl)ureido)-3- (methylthio)phenoxy)-N- phenylpicolinamide |
| (cp) | 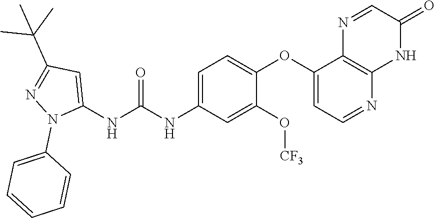 [SMILES: CC(C)(C)c1cc(NC(=O)Nc2ccc(Oc3ccnc4[nH]c(=O)cnc34)c(OC(F)(F)F)c2)n(-c2ccccc2)n1] | 1-(3-(tert-butyl)-1-phenyl-1H- pyrazol-5-yl)-3-(4-((3-oxo-3,4- dihydropyrido[2,3-b]pyrazin-8- yl)oxy)-3- (trifluoromethoxy)phenyl)urea |
| (cq) | 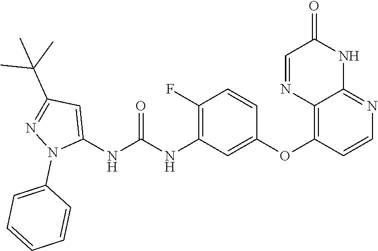 [SMILES: CC(C)(C)c1cc(NC(=O)Nc2cc(Oc3ccnc4[nH]c(=O)cnc34)ccc2F)n(-c2ccccc2)n1] | 1-(3-(tert-butyl)-1-phenyl-1H- pyrazol-5-yl)-3-(2-fluoro-5-((3-oxo- 3,4-dihydropyrido[2,3-b]pyrazin-8- yl)oxy)phenyl)urea |
| (cr) | 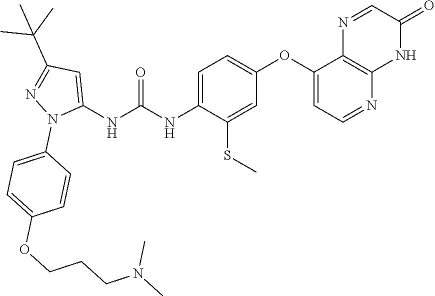 [SMILES: CSc1cc(Oc2ccnc3[nH]c(=O)cnc23)ccc1NC(=O)Nc1cc(C(C)(C)C)nn1-c1ccc(OCCCN(C)C)cc1] | 1-(3-(tert-butyl)-1-(4-(3- (dimethylamino)propoxy)phenyl)- 1H-pyrazol-5-yl)-3-(2-(methylthio)- 4-((3-oxo-3,4-dihydropyrido[2,3- b]pyrazin-8-yl)oxy)phenyl)urea |
| (cs) | 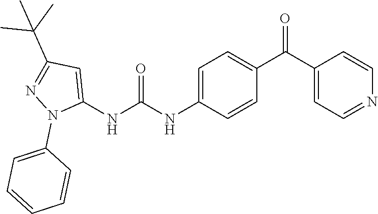 [SMILES: CC(C)(C)c1cc(NC(=O)Nc2ccc(C(=O)c3ccncc3)cc2)n(-c2ccccc2)n1] | 1-(3-(tert-butyl)-1-phenyl-1H- pyrazol-5-yl)-3-(4- isonicotinoylphenyl)urea |
| (ct) | 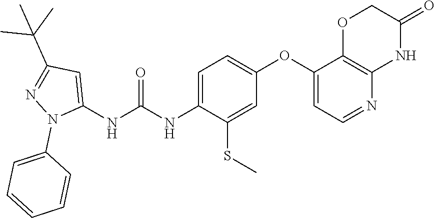 [SMILES: CSc1cc(Oc2ccnc3c2OCC(=O)N3)ccc1NC(=O)Nc1cc(C(C)(C)C)nn1-c1ccccc1] | 1-(3-(tert-butyl)-1-phenyl-1H- pyrazol-5-yl)-3-(2-(methylthio)-4- ((3-oxo-3,4-dihydro-2H-pyrido[3,2- b][1,4]oxazin-8-yl)oxy)phenyl)urea |
| (cu) | 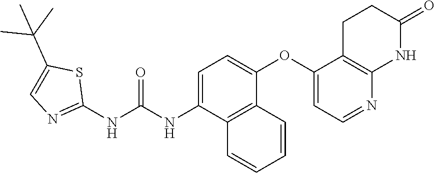 [SMILES: CC(C)(C)c1cnc(NC(=O)Nc2ccc(Oc3ccnc4c3CCC(=O)N4)c3ccccc23)s1] | 1-(5-(tert-butyl)thiazol-2-yl)-3-(4- ((7-oxo-5,6,7,8-tetrahydro-1,8- naphthyridin-4-yl)oxy)naphthalen-1- yl)urea |
| (cv) | 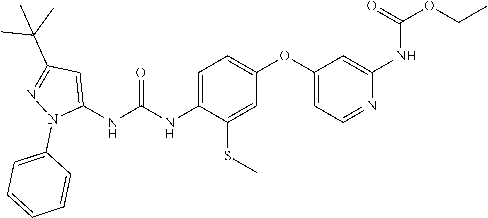 [SMILES: CCOC(=O)Nc1cc(Oc2ccc(NC(=O)Nc3cc(C(C)(C)C)nn3-c3ccccc3)c(SC)c2)ccn1] | ethyl (4-(4-(3-(3-(tert-butyl)- phenyl-1H-pyrazol-5-yl)ureido)-3- (methylthio)phenoxy)pyridin-2- yl)carbamate |
| (cw) | 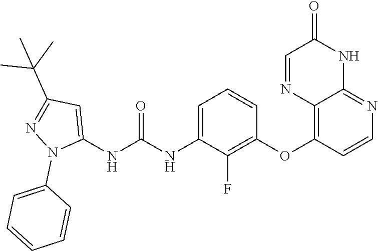 [SMILES: CC(C)(C)c1cc(NC(=O)Nc2cccc(Oc3ccnc4[nH]c(=O)cnc34)c2F)n(-c2ccccc2)n1] | 1-(3-(tert-butyl)-1-phenyl-1H- pyrazol-5-yl)-3-(2-fluoro-3-((3-oxo- 3,4-dihydropyrido[2,3-b]pyrazin-8- yl)oxy)phenyl)urea |
| (cx) | 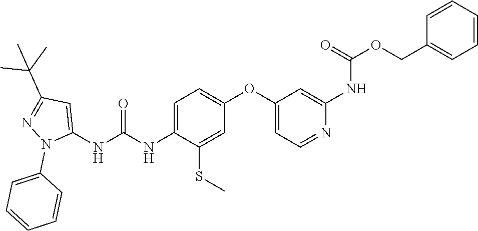 [SMILES: CSc1cc(Oc2ccnc(NC(=O)OCc3ccccc3)c2)ccc1NC(=O)Nc1cc(C(C)(C)C)nn1-c1ccccc1] | benzyl (4-(4-(3-(3-(tert-butyl)- phenyl-1H-pyrazol-5-yl)ureido)-3- (methylthio)phenoxy)pyridin-2- yl)carbamate |
| (cy) | 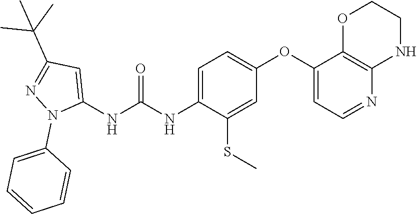 [SMILES: CSc1cc(Oc2ccnc3c2OCCN3)ccc1NC(=O)Nc1cc(C(C)(C)C)nn1-c1ccccc1] | 1-(3-(tert-butyl)-1-phenyl-1H- pyrazol-5-yl)-3-(4-((3,4-dihydro-2H- pyrido[3,2-b][1,4]oxazin-8-yl)oxy)- 2-(methylthio)phenyl)urea |
| (cz) | 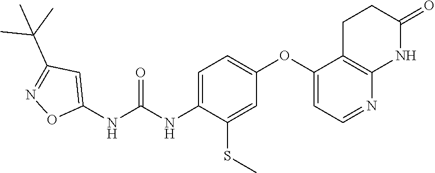 [SMILES: CSc1cc(Oc2ccnc3c2CCC(=O)N3)ccc1NC(=O)Nc1cc(C(C)(C)C)no1] | 1-(3-(tert-butyl)isoxazol-5-yl)-3-(2- (methylthio)-4-((7-oxo-5,6,7,8- tetrahydro-1,8-naphthyridin-4- yl)oxy)phenyl)urea |
| (da) | 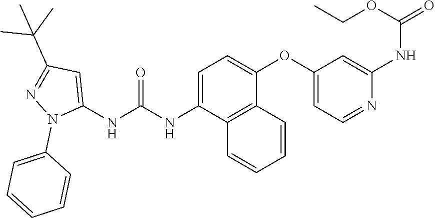 [SMILES: CCOC(=O)Nc1cc(Oc2ccc(NC(=O)Nc3cc(C(C)(C)C)nn3-c3ccccc3)c3ccccc23)ccn1] | ethyl (4-((4-(3-(3-(tert-butyl)- phenyl-1H-pyrazol-5- yl)ureido)naphthalen-1- yl)oxy)pyridin-2-yl)carbamate |
| (db) | 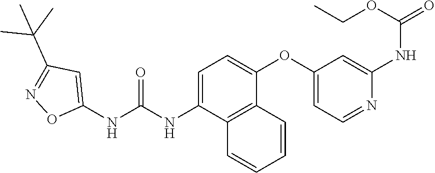 [SMILES: CCOC(=O)Nc1cc(Oc2ccc(NC(=O)Nc3cc(C(C)(C)C)no3)c3ccccc23)ccn1] | ethyl (4-((4-(3-(3-(tert- butyl)isoxazol-5- yl)ureido)naphthalen-1- yl)oxy)pyridin-2-yl)carbamate |
| (dc) | 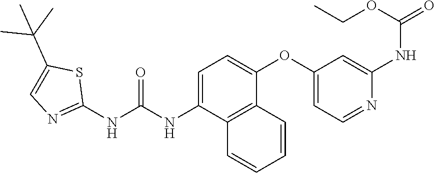 [SMILES: CCOC(=O)Nc1cc(Oc2ccc(NC(=O)Nc3ncc(C(C)(C)C)s3)c3ccccc23)ccn1] | ethyl (4-((4-(3-(5-(tert-butyl)thiazol- 2-yl)ureido)naphthalen-1- yl)oxy)pyridin-2-yl)carbamate |
| (dd) | 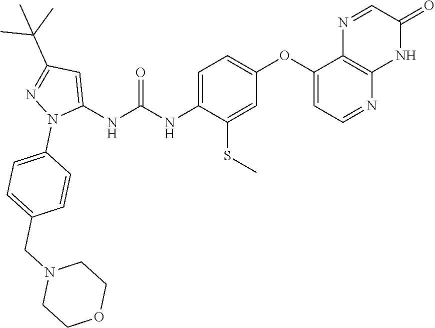 [SMILES: CSc1cc(Oc2ccnc3[nH]c(=O)cnc23)ccc1NC(=O)Nc1cc(C(C)(C)C)nn1-c1ccc(CN2CCOCC2)cc1] | 1-(3-(tert-butyl)-1-(4- (morpholinomethyl)phenyl)-1H- pyrazol-5-yl)-3-(2-(methylthio)-4- ((3-oxo-3,4-dihydropyrido[2,3- b]pyrazin-8-yl)oxy)phenyl)urea |
| (de) | 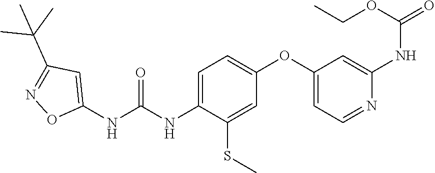 [SMILES: CCOC(=O)Nc1cc(Oc2ccc(NC(=O)Nc3cc(C(C)(C)C)no3)c(SC)c2)ccn1] | ethyl (4-(4-(3-(3-(tert-butyl)isoxazol- 5-yl)ureido)-3- (methylthio)phenoxy)pyridin-2- yl)carbamate |
| (df) | 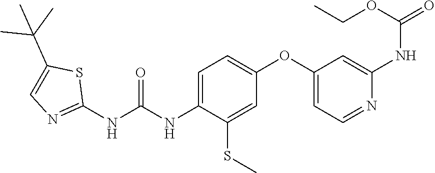 [SMILES: CCOC(=O)Nc1cc(Oc2ccc(NC(=O)Nc3ncc(C(C)(C)C)s3)c(SC)c2)ccn1] | ethyl (4-(4-(3-(5-(tert-butyl)thiazol- 2-yl)ureido)-3- (methylthio)phenoxy)pyridin-2- yl)carbamate |
| (dg) | 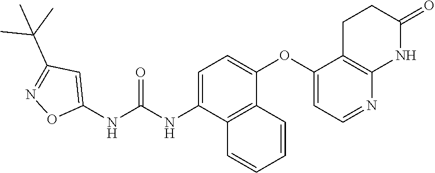 [SMILES: CC(C)(C)c1cc(NC(=O)Nc2ccc(Oc3ccnc4c3CCC(=O)N4)c3ccccc23)on1] | 1-(3-(tert-butyl)isoxazol-5-yl)-3-(4- ((7-oxo-5,6,7,8-tetrahydro-1,8- naphthyridin-4-yl)oxy)naphthalen-1- yl)urea |
| (dh) | 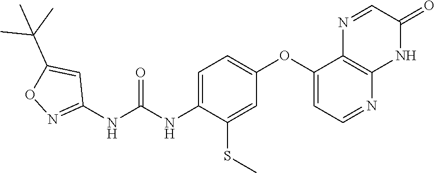 [SMILES: CSc1cc(Oc2ccnc3[nH]c(=O)cnc23)ccc1NC(=O)Nc1cc(C(C)(C)C)on1] | 1-(5-(tert-butyl)isoxazol-3-yl)-3-(2- (methylthio)-4-((3-oxo-3,4- dihydropyrido[2,3-b]pyrazin-8- yl)oxy)phenyl)urea |
| (di) | 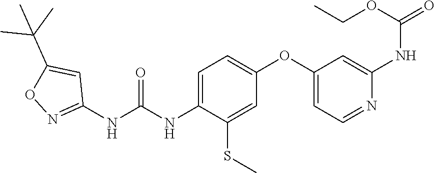 [SMILES: CCOC(=O)Nc1cc(Oc2ccc(NC(=O)Nc3cc(C(C)(C)C)on3)c(SC)c2)ccn1] | ethyl (4-(4-(3-(5-(tert-butyl)isoxazol- 3-yl)ureido)-3- (methylthio)phenoxy)pyridin-2- yl)carbamate |
| (dj) | 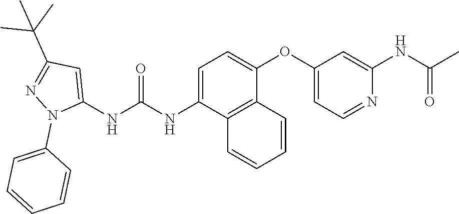 [SMILES: CC(=O)Nc1cc(Oc2ccc(NC(=O)Nc3cc(C(C)(C)C)nn3-c3ccccc3)c3ccccc23)ccn1] | N-(4-((4-(3-(3-(tert-butyl)-1-phenyl- 1H-pyrazol-5-yl)ureido)naphthalen- 1-yl)oxy)pyridin-2-yl)acetamide |
| (dk) | 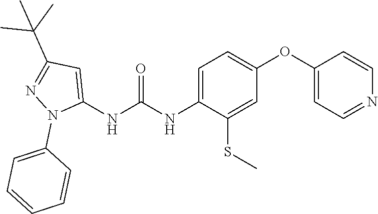 [SMILES: CSc1cc(Oc2ccncc2)ccc1NC(=O)Nc1cc(C(C)(C)C)nn1-c1ccccc1] | 1-(3-(tert-butyl)-1-phenyl-1H- pyrazol-5-yl)-3-(2-(methylthio)-4- (pyridin-4-yloxy)phenyl)urea |
| (dl) | 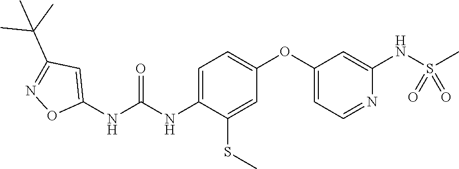 [SMILES: CSc1cc(Oc2ccnc(NS(C)(=O)=O)c2)ccc1NC(=O)Nc1cc(C(C)(C)C)no1] | N-(4-(4-(3-(3-(tert-butyl)isoxazol-5- yl)ureido)-3- (methylthio)phenoxy)pyridin-2- yl)methanesulfonamide |
| (dm) | 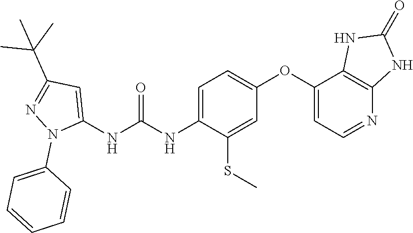 [SMILES: CSc1cc(Oc2ccnc3[nH]c(=O)[nH]c23)ccc1NC(=O)Nc1cc(C(C)(C)C)nn1-c1ccccc1] | 1-(3-(tert-butyl)-1-phenyl-1H- pyrazol-5-yl)-3-(2-(methylthio)-4- ((2-oxo-2,3-dihydro-1H-imidazo[4,5- b]pyridin-7-yl)oxy)phenyl)urea |
| (dn) | 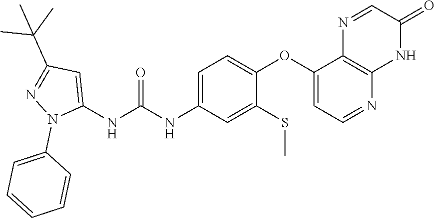 [SMILES: CSc1cc(NC(=O)Nc2cc(C(C)(C)C)nn2-c2ccccc2)ccc1Oc1ccnc2[nH]c(=O)cnc12] | 1-(3-(tert-butyl)-1-phenyl-1H- pyrazol-5-yl)-3-(3-(methylthio)-4- ((3-oxo-3,4-dihydropyrido[2,3- b]pyrazin-8-yl)oxy)phenyl)urea |
| (do) | 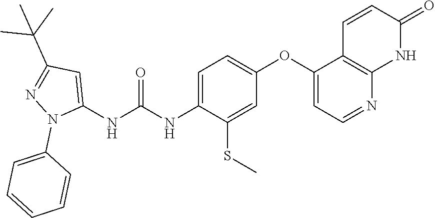 [SMILES: CSc1cc(Oc2ccnc3[nH]c(=O)ccc23)ccc1NC(=O)Nc1cc(C(C)(C)C)nn1-c1ccccc1] | 1-(3-(tert-butyl)-1-phenyl-1H- pyrazol-5-yl)-3-(2-(methylthio)-4- ((7-oxo-7,8-dihydro-1,8- naphthyridin-4-yl)oxy)phenyl)urea |
| (dp) | 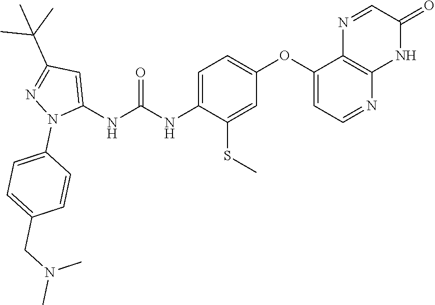 [SMILES: CSc1cc(Oc2ccnc3[nH]c(=O)cnc23)ccc1NC(=O)Nc1cc(C(C)(C)C)nn1-c1ccc(CN(C)C)cc1] | 1-(3-(tert-butyl)-1-(4- ((dimethylamino)methyl)phenyl)-1H- pyrazol-5-yl)-3-(2-(methylthio)-4- ((3-oxo-3,4-dihydropyrido[2,3- b]pyrazin-8-yl)oxy)phenyl)urea |
| (dq) | 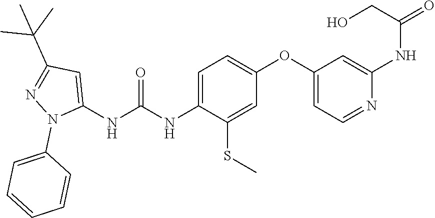 [SMILES: CSc1cc(Oc2ccnc(NC(=O)CO)c2)ccc1NC(=O)Nc1cc(C(C)(C)C)nn1-c1ccccc1] | N-(4-(4-(3-(3-(tert-butyl)-1-phenyl- 1H-pyrazol-5-yl)ureido)-3- (methylthio)phenoxy)pyridin-2-yl)-2- hydroxyacetamide. |

78. A compound of structure (I), 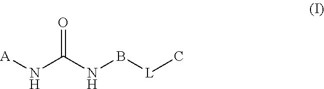
wherein
L is —O—; and
a) A is a ring system selected from the group consisting of 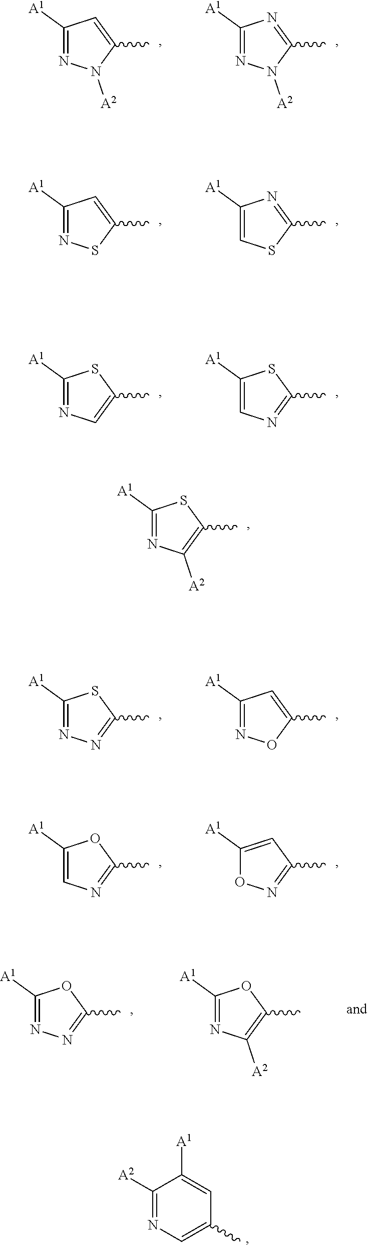
A¹ is selected from the group consisting of methyl, —CF₃, isopropyl, cyclopropyl, isobutyl, sec-butyl, tert-butyl, 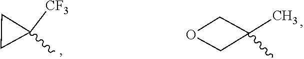
and A² is selected from the group consisting of H, methyl, iso-propyl, benzyl, phenyl, chlorine, 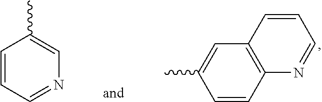
wherein A¹ and A² are optionally fluorinated, and A₂ is optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,
B is a ring system selected from the group consisting of 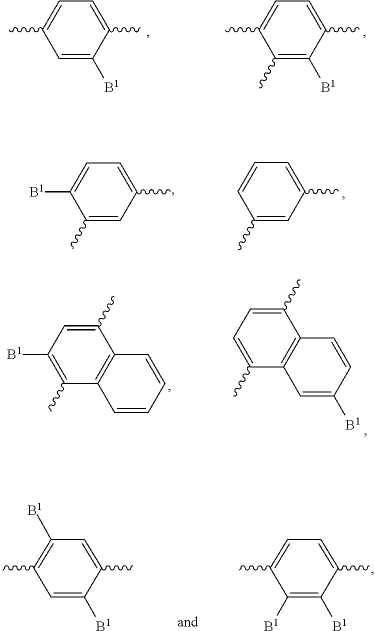
wherein, when B is a phenyl, B¹ is selected from the group consisting of —F, —Cl, —SCH₃, —SCH₂CH₃, —CH₃, —CF₃, —OCH₃, and —OCF₃ and, when B is a naphtyl, B¹ is —H, and
C is selected from the group consisting of 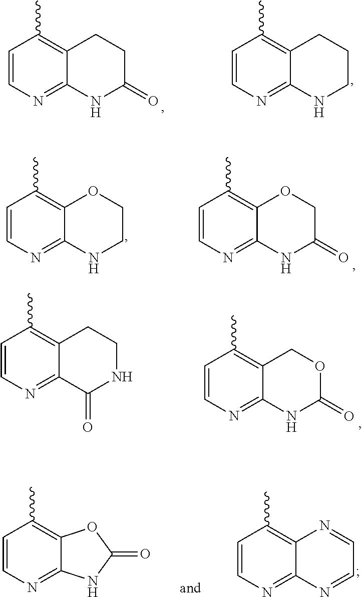
or
wherein A is a ring selected from the group consisting of 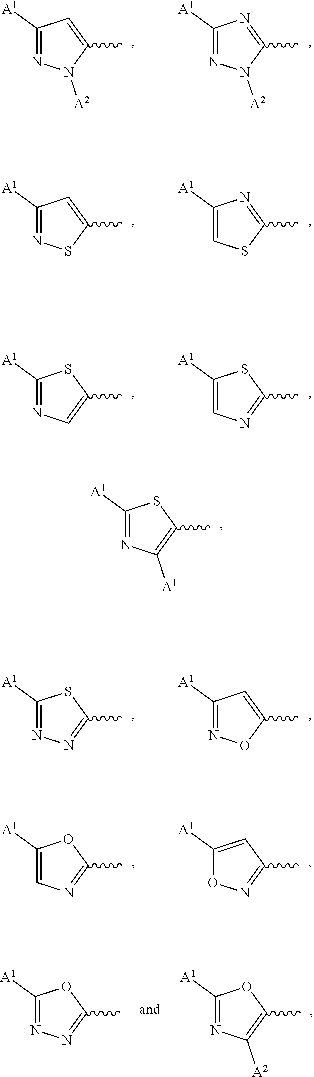
A¹ is tert-butyl,
A² is a phenyl or 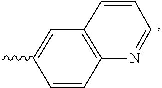
optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,
B is a ring system selected from the group consisting of 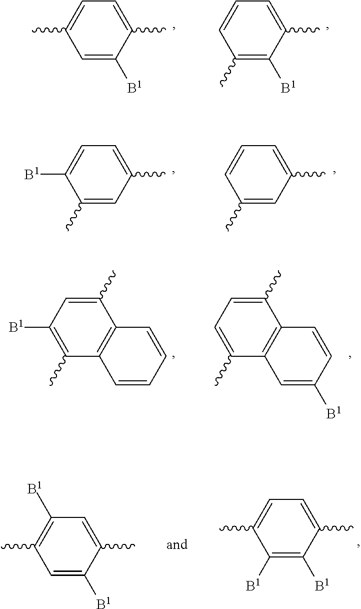
wherein, when B is a phenyl, B¹ is selected from the group consisting of —F, —SCH₃, —SCH₂CH₃, —CF₃, —OCH₃, and —OCF₃ and, when B is a naphtyl, B¹ is —H, and
C is selected from the group consisting of 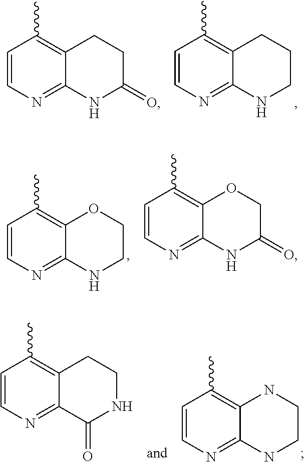
or
wherein A is a ring selected from the group consisting of 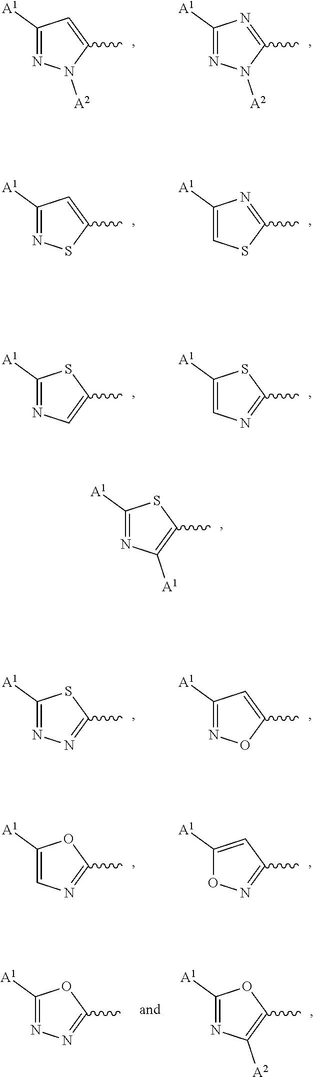
A¹ is tert-butyl,
A² is a phenyl or 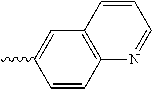
optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,
B is a ring system selected from the group consisting of 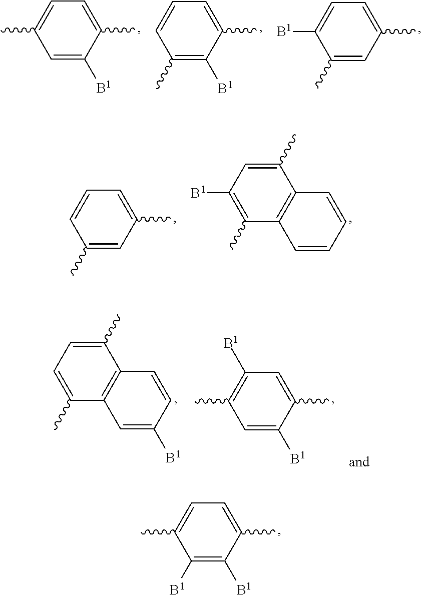
wherein, when B is a phenyl, B¹ is selected from the group consisting of —F, —SCH₃, —SCH₂CH₃, —CF₃, —OCH₃, and —OCF₃ and, when B is a naphtyl, B¹ is —H, and
C is selected from the group consisting of 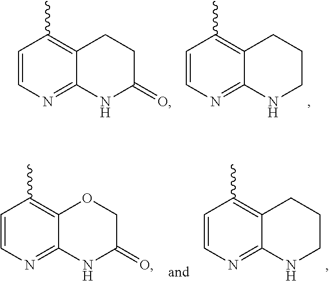
or
wherein A is a ring selected from the group consisting of 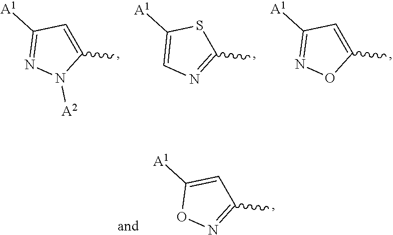
A¹ is tert-butyl,
A² is a phenyl optionally substituted with 1, 2 or 3 substituents selected from the group consisting of methyl, —Cl, —F, —CN, methoxy, —O—(CH₂)₂-piperidinyl, —O—(CH₂)₂-morpholinyl, —O—(CH₂)₃—N(CH₃)₂, and —CH₂-morpholinyl, or A² is 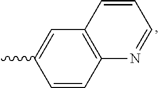
B is a ring system selected in the group consisting of 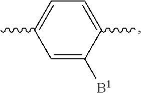
with B¹ being selected from the group consisting of —F, —SCH₃, —SCH₂CH₃, —CF₃, —OCH₃, and —OCF₃, preferably —SCH₃, 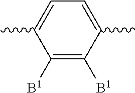
with B¹ being F,
and 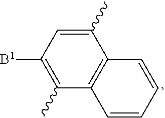
with B¹ being —H, and
C is selected from the group consisting of 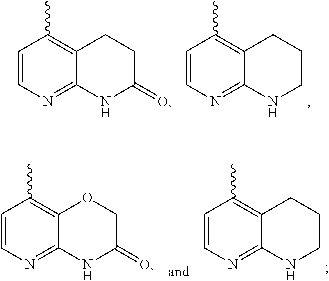
b) A is a ring selected from the group consisting of 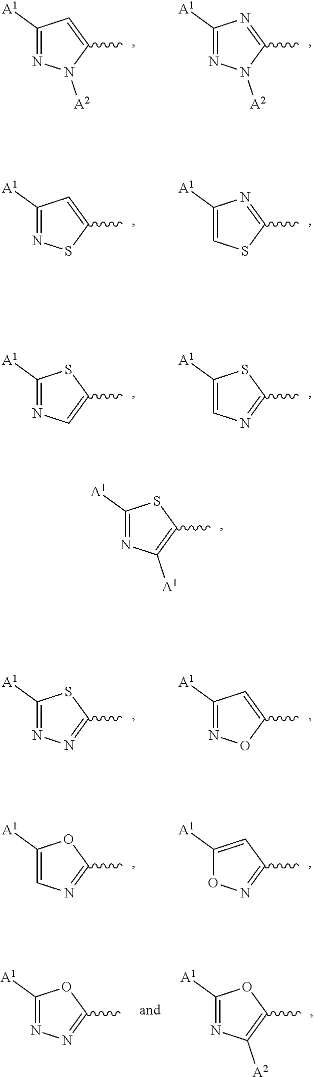
A¹ is selected from the group consisting of methyl, —CF₃, isopropyl, cyclopropyl, isobutyl, sec-butyl, tert-butyl, 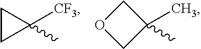
A² is —H,
B is a ring system selected from the group consisting of 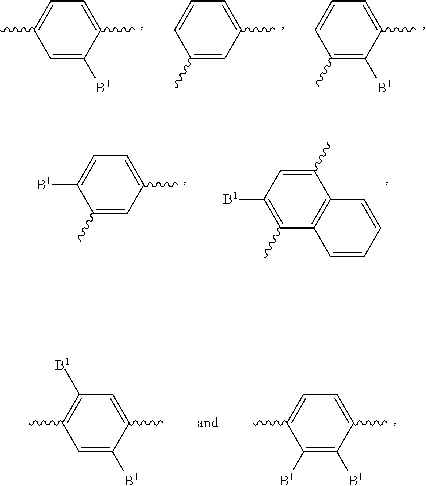
wherein, when B is a phenyl, B¹ is selected from the group consisting of —F, —Cl, —SCH₃, —SCH₂CH₃, —CH₃, —CF₃, —OCH₃, and —OCF₃ and, when B is a naphtyl, B¹ is —H, and
C is selected from the group consisting of 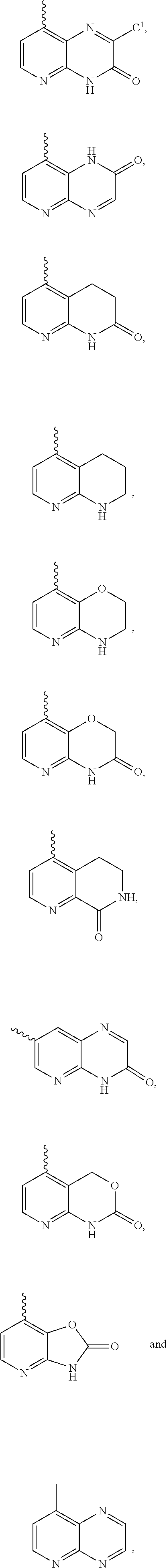
wherein C¹ is selected from the group consisting of —H, —CH₃, —OCH₃, —CN or 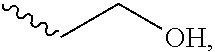
or
C is selected from the group consisting of 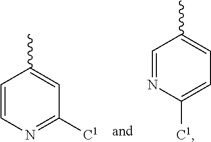
with C¹ being selected C¹ is selected from the group consisting of wherein C¹ is selected from the group consisting of —H, —NH₂, —NR¹, —C(O)—NH—R¹, —NH—C(O)—R¹, —NH—S(O)₂—R¹, —NH—C(O)—OR¹, —C(O)—R¹, —R¹, —OR¹, —SO₂R¹, with R¹ being selected from the group consisting of C₁-C₁₀ alkyl, C₆-C₁₄ aryl, C₃-C₁₃ heteroaryl, and C₇-C₂₄ alkaryl, wherein each hydrogen in each of these groups may be independently replaced by a group selected from halogen, hydroxy, —OR′, and —NR′R″, each hydrogen in each of R′ and R″ may be independently replaced by —COR′″ or COOR′″, wherein R′, R″ and R′″ are independently a C₁-C₆ alkyl,
or
wherein A is a ring selected from the group consisting of 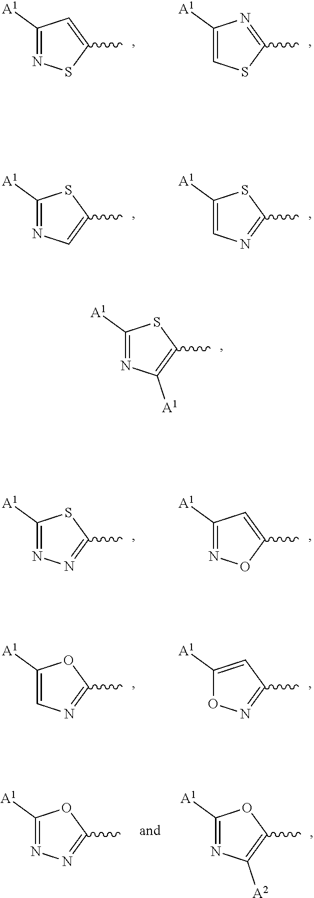
A¹ is tert-butyl,
A² is —H,
B is a ring system selected from the group consisting of 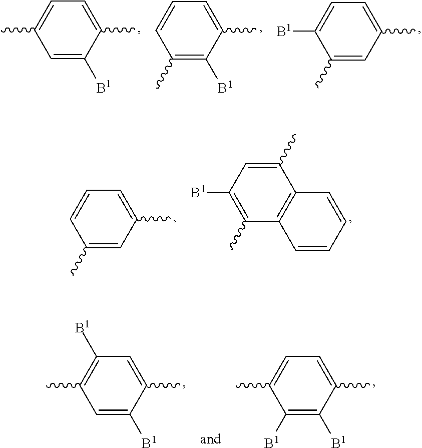
wherein, when B is a phenyl, B¹ is selected from the group consisting of —F, —SCH₃, —SCH₂CH₃, —CF₃, —OCH₃, and —OCF₃ and, when B is a naphtyl, B¹ is —H, and
C is selected from the group consisting of 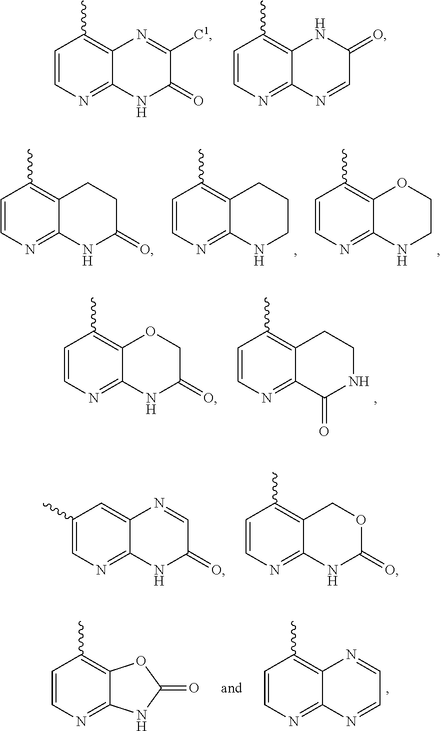
wherein C¹ is selected from the group consisting of —H, —CH₃, —OCH₃, —CN or 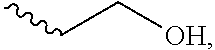
or
C is selected from the group consisting of 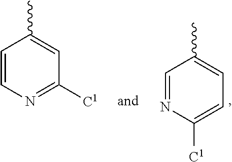
with C¹ being selected from the group consisting of —H, —NH₂, 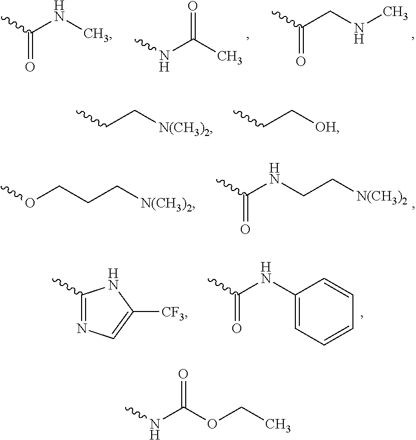
—NH—C(O)OtBu, —NH—C(O)N(CH3)-C(O)OtBu, —NH—C(O)—CH₂—N(CH₃)—C(O)OtBu, —NH—C(O)—CH₂—NHCH₃, —NH—C(O)—Obenzyl, —NH—C(O)—CH₂—OH, —NH—(CH₂)₂—N(CH₃)₂, and —NH—S(O)₂—CH₃, preferably from the group consisting of —H, 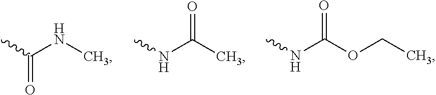
—NH—C(O)OtBu, —NH—C(O)N(CH₃)—C(O)OtBu, —NH—C(O)—CH₂—N(CH₃)—C(O)OtBu, —NH—C(O)—CH₂—NHCH₃, —NH—C(O)—Obenzyl, and —NH—C(O)—CH₂—OH;
or
wherein A is selected in the group consisting of 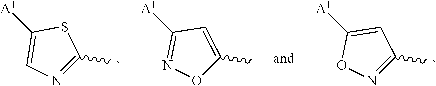
A¹ is tert-butyl,
B is a ring system selected in the group consisting of 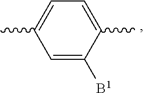
with B¹ being selected from the group consisting of —F, —SCH₃, —SCH₂CH₃, —CF₃, —OCH₃, and —OCF₃, preferably —SCH₃, 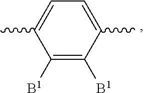
with B¹ being F,
and 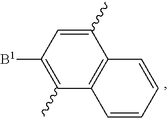
with B¹ being —H, and
C is selected in the group consisting of 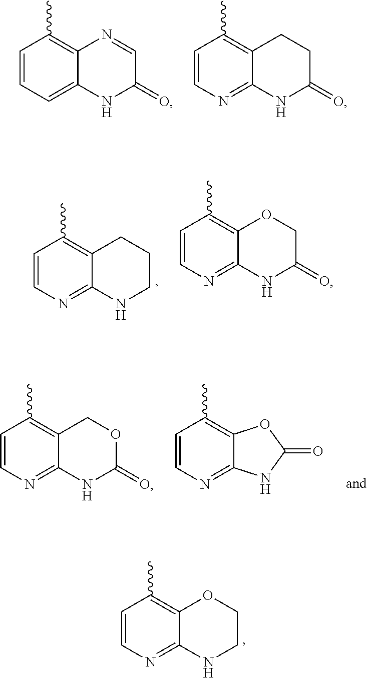
or C is selected from the group consisting of 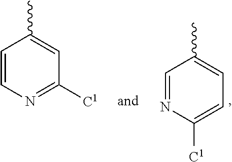
with C¹ being selected from the group consisting of 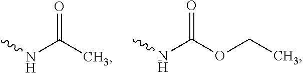
—NH—C(O)OtBu, —NH—C(O)N(CH3)-COOtBu and —NH—C(O)—CH₂—N(CH₃)—C(O)OtBu;
c) A is a ring system selected from the group consisting of 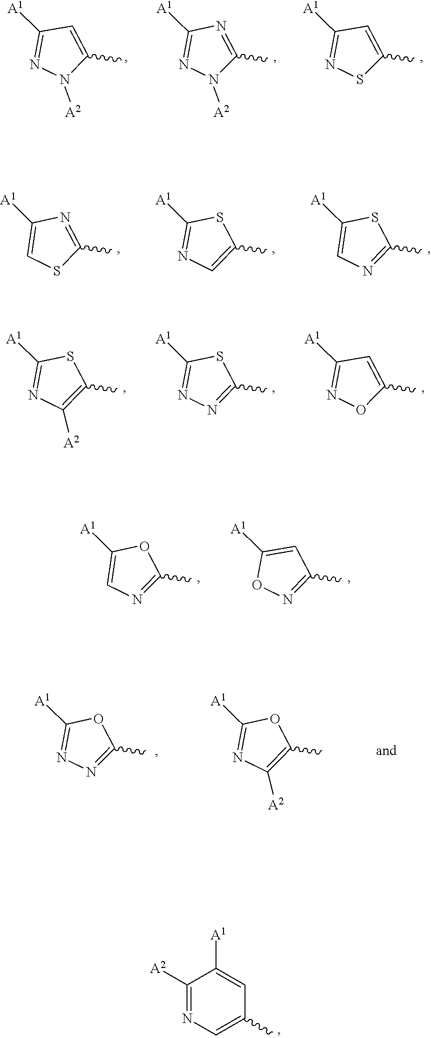
A¹ is selected from the group consisting of methyl, —CF₃, isopropyl, cyclopropyl, isobutyl, sec-butyl, tert-butyl, 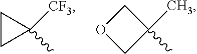
and A² is selected from the group consisting of H, methyl, iso-propyl, benzyl, phenyl, chlorine, 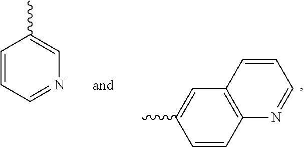
wherein A¹ and A² are optionally fluorinated, and A₂ is optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,
B is a ring system selected from the group consisting of 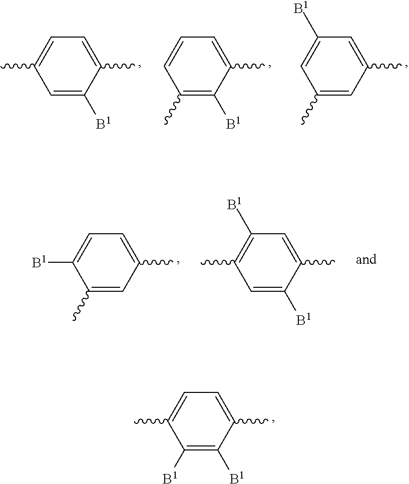
wherein B¹ is selected from the group consisting of —CF₃, —OCF₃, —OCH₃, and SCH₂CH₃, or B has two B¹ groups selected independently from the group consisting of —F, —Cl, —SCH₃, —SCH₂CH₃, —CH₃, isopropyl, —CF₃, —OCH₃, —OCF₃,
C is selected from the group consisting of 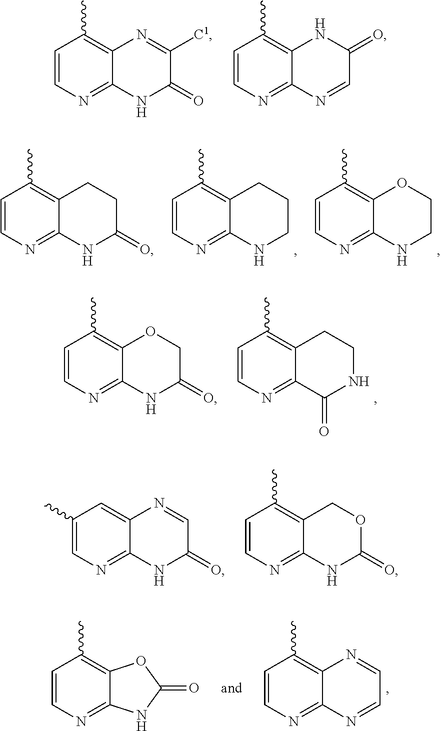
wherein C¹ is selected from the group consisting of —H, —CH₃, —OCH₃, —CN or 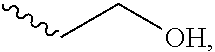
or
C is selected from the group consisting of 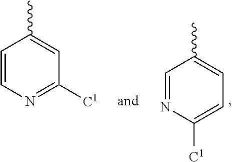
with C¹ being selected C¹ is selected from the group consisting of wherein C¹ is selected from the group consisting of —H, —NH₂, —NR′, —C(O)—NH—R¹, —NH—C(O)—R¹, —NH—S(O)₂—R¹, —NH—C(O)—OR¹, —C(O)—R¹, —R¹, —OR¹, —SO₂R¹, with R¹ being selected from the group consisting of C₁-C₁₀ alkyl, C₆-C₁₄ aryl, C₃-C₁₃ heteroaryl, and C₇-C₂₄ alkaryl, wherein each hydrogen in each of these groups may be independently replaced by a group selected from a halogen, a hydroxy, —OR′, and —NR′R″, each hydrogen in each of R′ and R″ may be independently replaced by —COR′″ or COOR′″, wherein R′, R″ and R′″ are independently a C₁-C₆ alkyl;
or
wherein A is a ring selected from the group consisting of 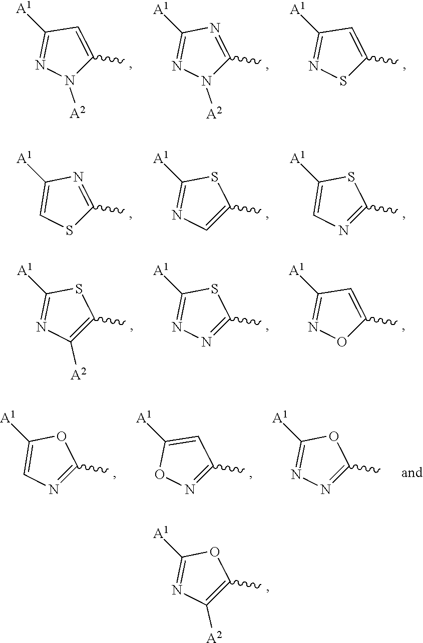
A¹ is tert-butyl,
A² is a phenyl or 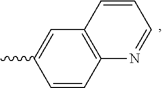
optionally substituted with 1, 2 or 3 substituents selected from the group consisting of C₁-C₄ alkyl, —Cl, —F, —CN, —OR^{ABC}, and —SO₂R^{ABC}, wherein R^{AC} is C₁-C₁₀ alkyl, and the substituent being optionally substituted by C₃-C₇ heterocycloalkyl or NRR, with R being a C₁-C₃ alkyl,
B is a ring system selected from the group consisting of 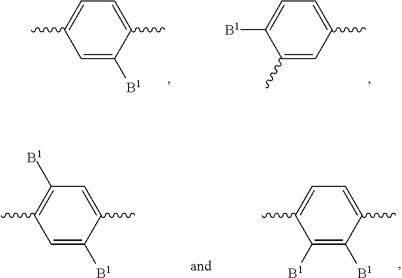
wherein B¹ is selected from the group consisting of —CF₃, —OCF₃, —OCH₃, and SCH₂CH₃, or B has two B¹ groups selected independently from the group consisting of —F, —Cl, —SCH₃, —SCH₂CH₃, —CH₃, isopropyl, —CF₃, —OCH₃, —OCF₃,
C is selected from the group consisting of 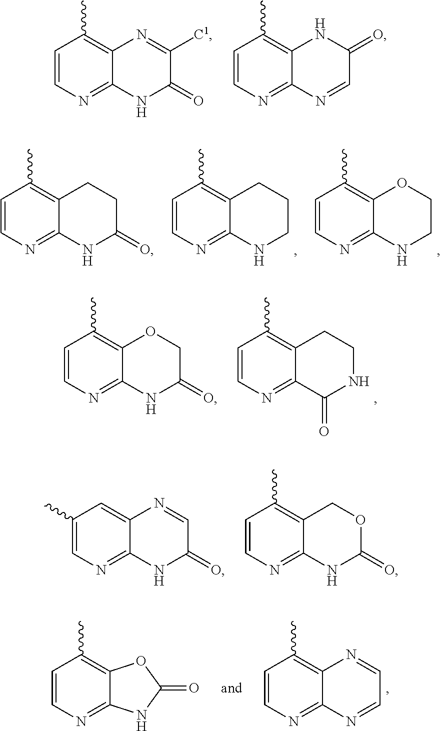
wherein C¹ is selected from the group consisting of —H, —CH₃, —OCH₃, —CN or 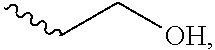
or
C is selected from the group consisting of 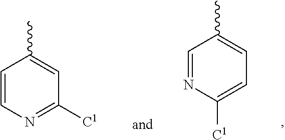
with C¹ being selected from the group consisting of —H, —NH₂, 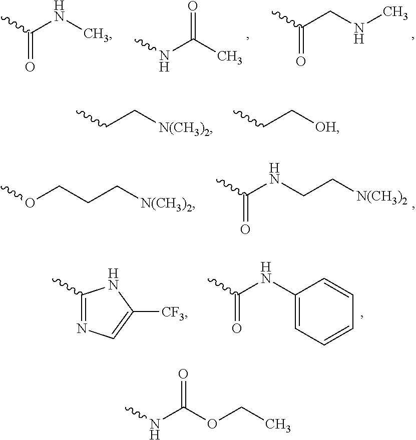
—NH—C(O)OtBu, —NH—C(O)N(CH3)-C(O)OtBu, —NH—C(O)—CH₂—N(CH₃)—C(O)OtBu, —NH—C(O)—CH₂—NHCH₃, —NH—C(O)—Obenzyl, —NH—C(O)—CH₂—OH, —NH—(CH₂)₂—N(CH₃)₂, and —NH—S(O)₂—CH₃, preferably from the group consisting of —H, 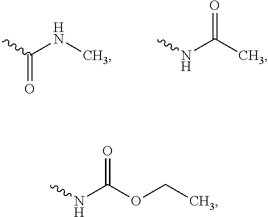
—NH—C(O)OtBu, —NH—C(O)N(CH₃)—C(O)OtBu, —NH—C(O)—CH₂—N(CH₃)—C(O)OtBu, —NH—C(O)—CH₂—NHCH₃, —NH—C(O)—Obenzyl, and —NH—C(O)—CH₂—OH,
or
wherein A is a ring selected from the group consisting of 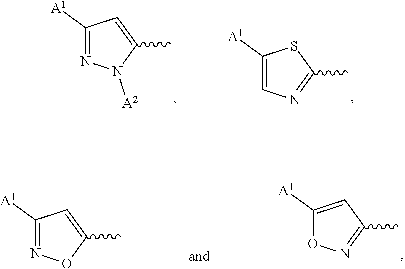
A¹ is tert-butyl,
A² is a phenyl optionally substituted with 1, 2 or 3 substituents selected from the group consisting of methyl, —Cl, —F, —CN, methoxy, —O—(CH₂)₂-piperidinyl, —O—(CH₂)₂-morpholinyl, —O—(CH₂)₃—N(CH₃)₂, and —CH₂-morpholinyl, or A² is 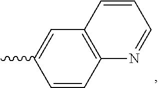
B is a ring system selected in the group consisting of 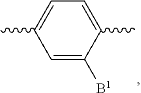
with B¹ being selected from the group consisting of —CF₃, —SCH₂CH₃, —OCH₃, and —OCF₃, preferably —CF₃ or —SCH₂CH₃, 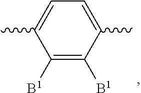
with B¹ being F,
C is selected in the group consisting of 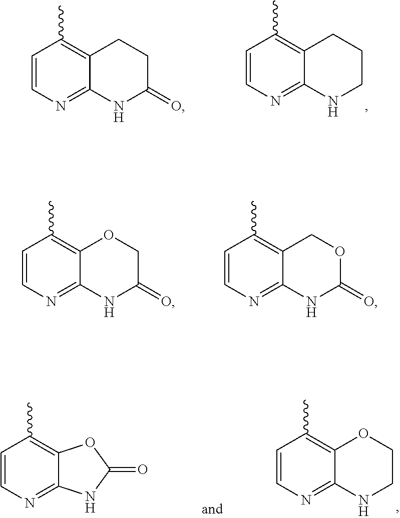
or C is selected from the group consisting of 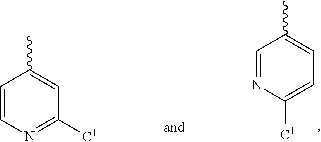
with C¹ being selected from the group consisting of 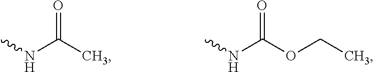
—NH—C(O)OtBu, —NH—C(O)—CH₂—N(CH₃)—C(O)OtBu and —NH—C(O)N(CH3)-COOtBu;
d) A is 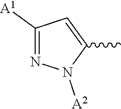
A¹ is selected from the group consisting of methyl, —CF₃, isopropyl, cyclopropyl, isobutyl, sec-butyl, tert-butyl, 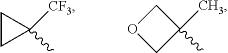
and A² is selected from the group consisting of a phenyl substituted with 1, 2 or 3 substituents selected from the group consisting of methoxy, —Cl, —F, —CN, —O—(CH₂)₂-piperidinyl, —O—(CH₂)₂-morpholinyl, —O—(CH₂)₃—N(CH₃)₂, and —CH₂-morpholinyl; a phenyl or pyridinyl substituted with 2 or 3 methyls, and a pyridinyl substituted with 1, 2 or 3 substituents selected from the group consisting of methyl, —Cl, —F, —CN, —O—(CH₂)₂-piperidinyl, —O—(CH₂)₂-morpholinyl, —O—(CH₂)₃—N(CH₃)₂, and —CH₂-morpholinyl;
B is a ring system selected from the group consisting of 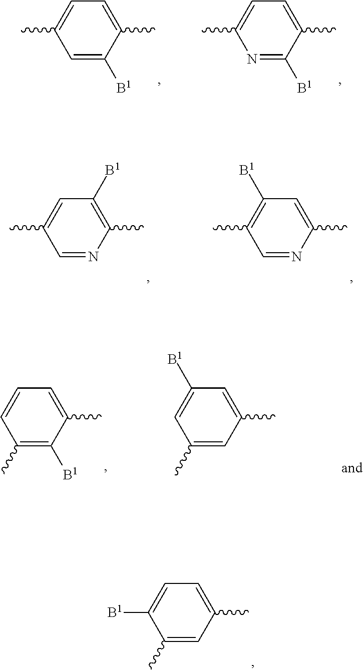
wherein B¹ is —SCH₃,
C is selected from the group consisting of 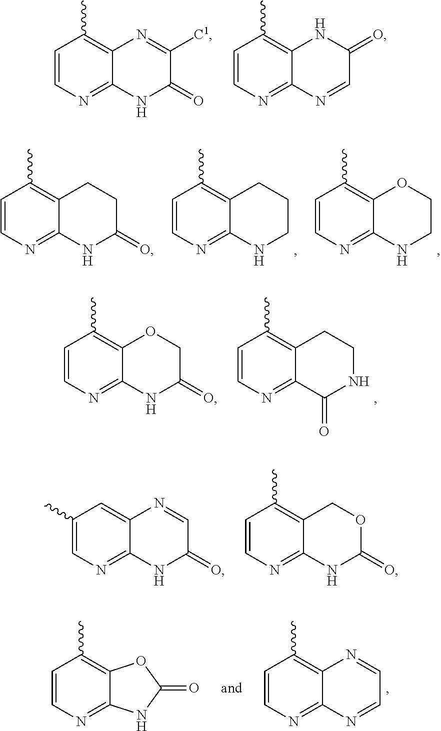
wherein C¹ is selected from the group consisting of —H, —CH₃, —OCH₃, —CN or 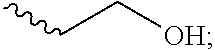
or
wherein A is 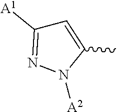
A¹ is tert-butyl,
and A² is selected from the group consisting of a phenyl substituted with 1, 2 or 3 substituents selected from the group consisting of methoxy, —Cl, —F, —CN, —O—(CH₂)₂-piperidinyl, —O—(CH₂)₂-morpholinyl, —O—(CH₂)₃—N(CH₃)₂, and —CH₂-morpholinyl; a phenyl or pyridinyl substituted with 2 or 3 methyls, and a pyridinyl substituted with 1, 2 or 3 substituents selected from the group consisting of methyl, —Cl, —F, —CN, —O—(CH₂)₂-piperidinyl, —O—(CH₂)₂-morpholinyl, —O—(CH₂)₃—N(CH₃)₂, and —CH₂-morpholinyl;
B is a ring system selected from the group consisting of 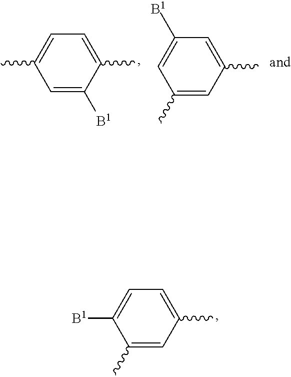
wherein B¹ is —SCH₃,
C is selected from the group consisting of 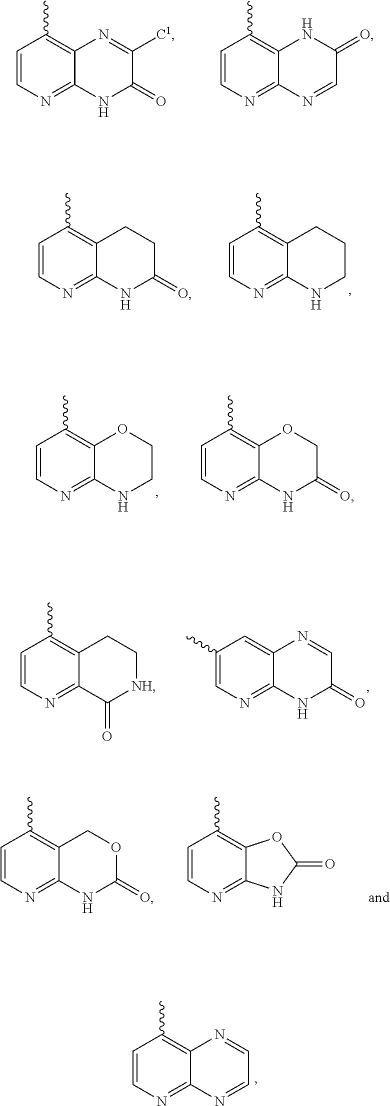
wherein C¹ is selected from the group consisting of —H, —CH₃, —OCH₃, —CN or 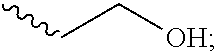
or
wherein A is 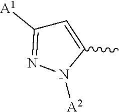
A¹ is tert-butyl,
and A² is selected from the group consisting of a phenyl substituted with 1, 2 or 3 substituents selected from the group consisting of —Cl, —F, —CN, —O—(CH₂)₂-piperidinyl, —O—(CH₂)₃—N(CH₃)₂, and —CH₂-morpholinyl; a pyridinyl substituted with a methyl; and a phenyl substituted by two methyls;
B is 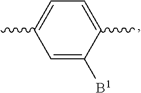
with B¹ being —SCH₃,
C is selected in the group consisting of 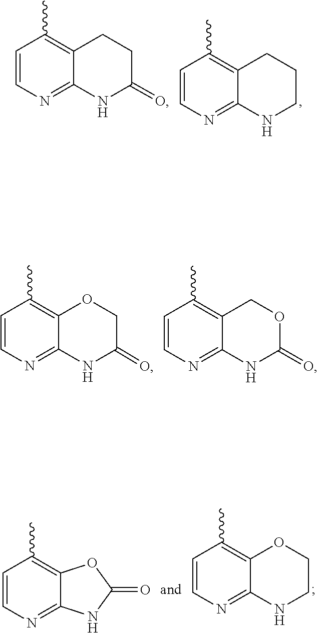
e)
A is 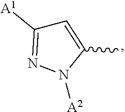
A¹ is selected from the group consisting of methyl, —CF₃, isopropyl, cyclopropyl, isobutyl, sec-butyl, tert-butyl, 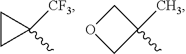
A² is 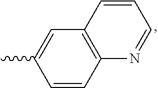
B is a ring system selected from the group consisting of 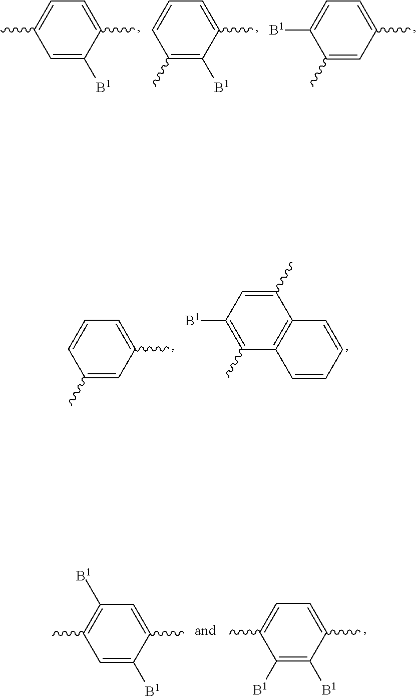
wherein, when B is a phenyl, B¹ is selected from the group consisting of —F, —Cl, —SCH₃, —SCH₂CH₃, —CH₃, —CF₃, —OCH₃, and —OCF₃ and, when B is a naphtyl, B¹ is —H, and
C is selected from the group consisting of 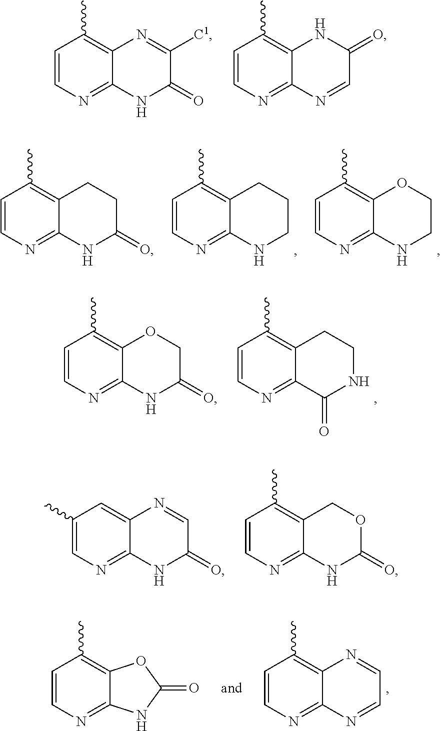
wherein C¹ is selected from the group consisting of —H, —CH₃, —OCH₃, —CN or 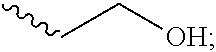
or
wherein A is 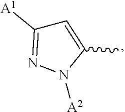
A¹ is tert-butyl,
A¹ is 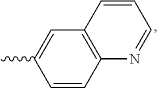
B is a ring system selected from the group consisting of 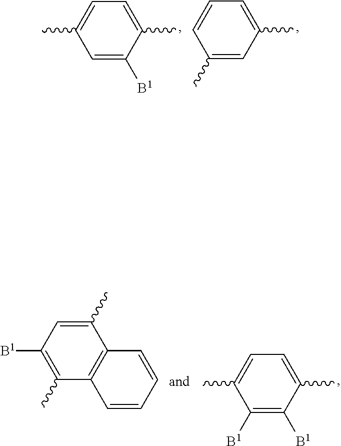
wherein, when B is a phenyl, B¹ is selected from the group consisting of —F, —Cl, —SCH₃, —SCH₂CH₃, —CH₃, —CF₃, —OCH₃, and —OCF₃ and, when B is a naphtyl, B¹ is —H, and
C is selected from the group consisting of 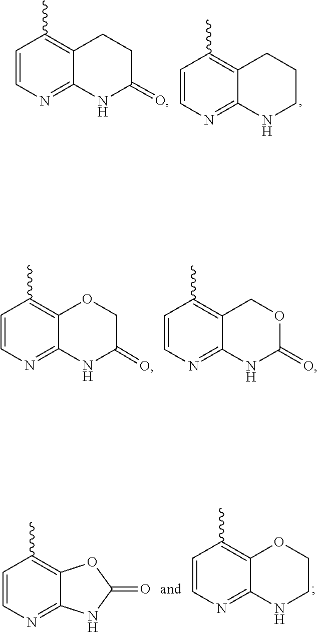
f)
A is a ring selected from the group consisting of 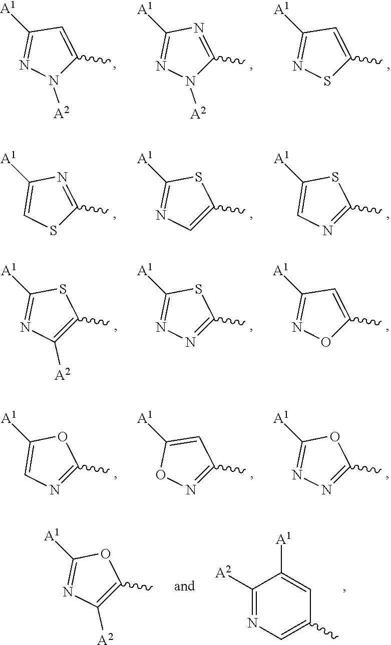
A¹ is selected from the group consisting of methyl, —CF₃, isopropyl, cyclopropyl, isobutyl, sec-butyl, tert-butyl, 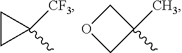
and A² is selected from the group consisting of H, methyl, iso-propyl, benzyl, phenyl, chlorine, 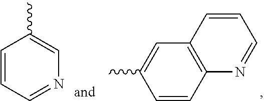
wherein A¹ and A² are optionally fluorinated, and A₂ is optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,
B is a ring system selected from the group consisting of 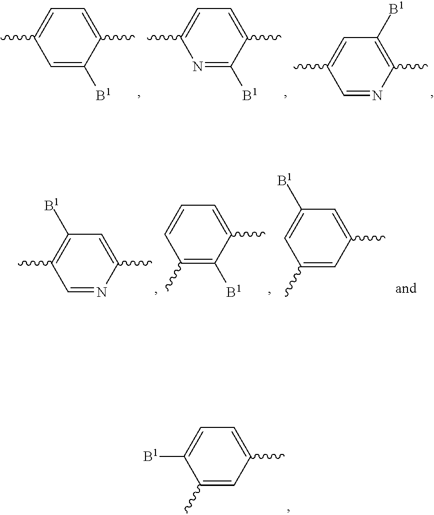
wherein B¹ is —SCH₃,
C is selected from the group consisting of 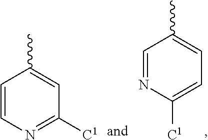
with C¹ being selected C¹ is selected from the group consisting of wherein C¹ is selected from the group consisting of —H, —NH₂, —NR¹, —C(O)—NH—R¹, —NH—S(O)₂—R¹, —NH—C(O)—OR¹, —C(O)—R¹, —R¹, —OR¹, —SO₂R¹, with R¹ being selected from the group consisting of C₁-C₁₀ alkyl, C₆-C₁₄ aryl, C₃-C₁₃ heteroaryl, and C₇-C₂₄ alkaryl, wherein each hydrogen in each of these groups may be independently replaced by a group selected from a halogen, a hydroxy, —OR′, and —NR′R″, each hydrogen in each of R′ and R″ may be independently replaced by —COR′″ or COOR′″, wherein R′, R″ and R′″ are independently a C₁-C₆ alkyl;
or
wherein A is a ring selected from the group consisting of 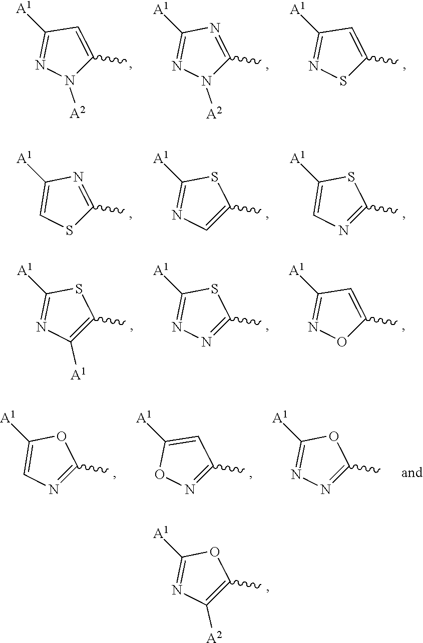
A¹ is tert-butyl,
A² is a phenyl or 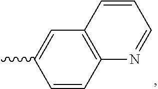
optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,
B is a ring system selected from the group consisting of 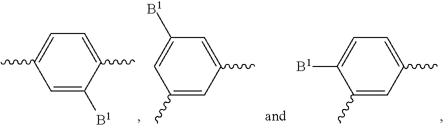
wherein B¹ is —SCH₃,
C is selected from the group consisting of 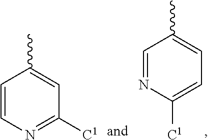
with C¹ being selected from the group consisting of —H, —NH₂, 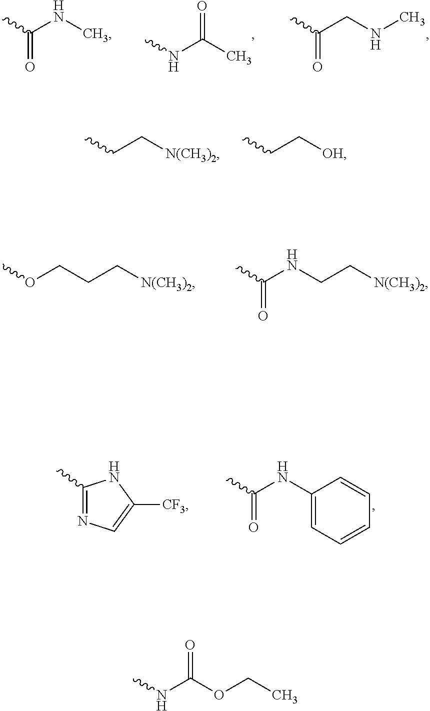
—NH—C(O)OtBu, —NH—C(O)N(CH3)-C(O)OtBu, —NH—C(O)—CH₂—N(CH₃)—C(O)OtBu, —NH—C(O)—CH₂—NHCH₃, —NH—C(O)—Obenzyl, —NH—C(O)—CH₂—OH, —NH—(CH₂)₂—N(CH₃)₂, and —NH—S(O)₂—CH₃, preferably from the group consisting of —H, 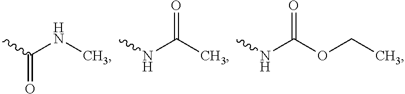
—NH—C(O)OtBu, —NH—C(O)N(CH₃)—C(O)OtBu, —NH—C(O)—CH₂—N(CH₃)—C(O)OtBu, —NH—C(O)—CH₂—NHCH₃, —NH—C(O)—Obenzyl, and —NH—C(O)—CH₂—OH;
or
wherein A is a ring selected from the group consisting of 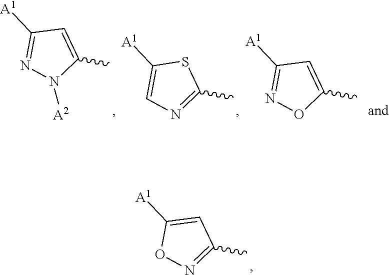
A¹ is tert-butyl,
A² is a phenyl optionally substituted with 1, 2 or 3 substituents selected from the group consisting of methyl, —Cl, —F, —CN, methoxy, —O—(CH₂)₂-piperidinyl, —O—(CH₂)₂-morpholinyl, —O—(CH₂)₃—N(CH₃)₂, and —CH₂-morpholinyl, or A² is 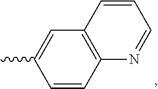
B is 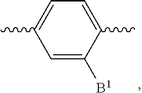
with B¹ being —SCH₃,
C is selected from the group consisting of 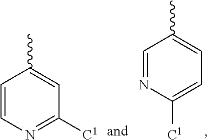
with C¹ being selected from the group consisting of 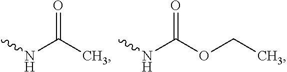
—NH—C(O)OtBu, —NH—C(O)—CH₂—N(CH₃)—C(O)OtBu and —NH—C(O)N(CH3)-COOtBu; or
g) A is a ring system selected from the group consisting of 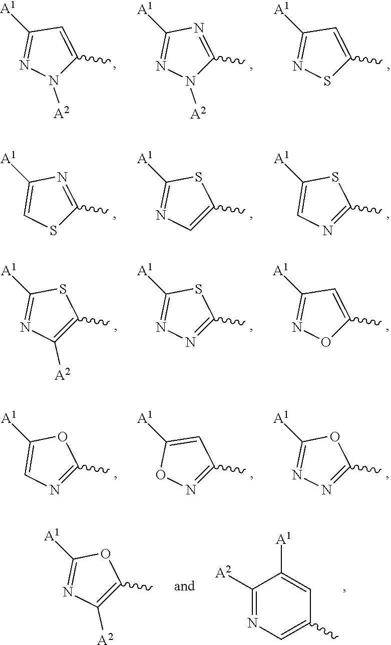
A¹ is selected from the group consisting of methyl, —CF₃, isopropyl, cyclopropyl, isobutyl, sec-butyl, tert-butyl, 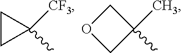
and A² is selected from the group consisting of H, methyl, iso-propyl, benzyl, phenyl, chlorine, 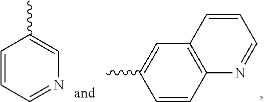
wherein A¹ and A² are optionally fluorinated, and A² is optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,
B is a ring system selected from the group consisting of 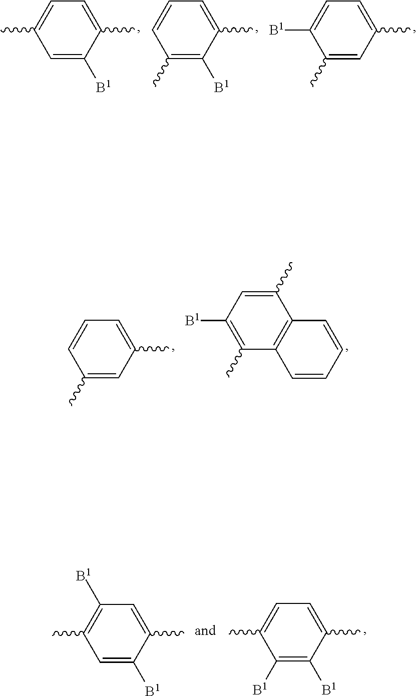
wherein, when B is a phenyl, B¹ is selected from the group consisting of —F, —Cl, —SCH₃, —SCH₂CH₃, —CH₃, —CF₃, —OCH₃, and —OCF₃ and, when B is a naphtyl, B¹ is —H, and
C is selected from the group consisting of 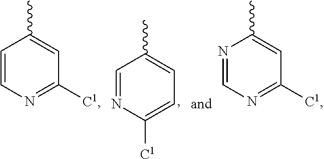
wherein C¹ is selected from the group consisting of —NH—C(O)R¹ or —NH—C(O)—OR¹ with R¹ being selected from the group consisting of C₁-C₁₀ alkyl, C₆-C₁₄ aryl, C₃-C₁₃ heteroaryl, and C₇-C₂₄ alkaryl, wherein each hydrogen in each of these groups may be independently replaced by a group selected from halogen, hydroxy, —OR′, and —NR′R″, each hydrogen in each of R′ and R″ may be independently replaced by —COR′ or COOR′″, wherein R′, R″ and R′″ are independently a C₁-C₆ alkyl, preferably selected from the group consisting of —NH—C(O)R¹ or —NH—C(O)—OR¹ with R¹ being selected from the group consisting of C₁-C₁₀ alkyl, C₆-C₁₄ aryl, C₃-C₁₃ heteroaryl, and C₇-C₂₄ alkaryl, wherein each hydrogen in each of these groups may be independently replaced by a group selected from halogen, hydroxy, and —NR′R″, each hydrogen in each of R′ and R″ may be independently replaced by —COR′″ or COOR′″, wherein R′, R″ and R′″ are independently a C₁-C₆ alkyl;
or
wherein A is a ring selected from the group consisting of 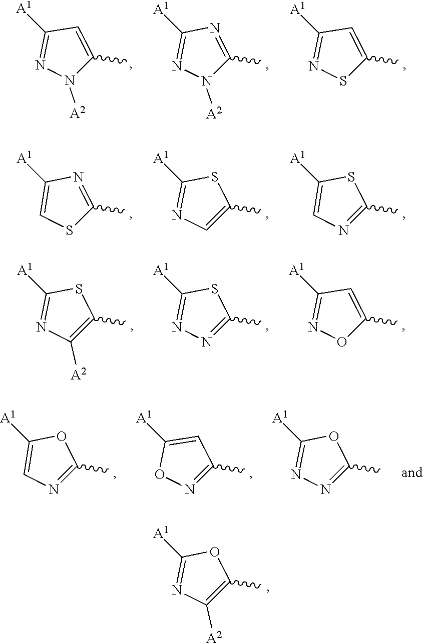
A¹ is tert-butyl,
A² is a phenyl or 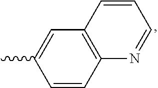
optionally substituted with 1, 2 or 3 substituents selected from the group consisting of C₁-C₄ alkyl, —Cl, —F, —CN, —OR^{ABC}, and —SO₂R^{ABC}, wherein R^{ABC} is C₁-C₁₀ alkyl, and the substituent being optionally substituted by C₃-C₇ heterocycloalkyl or NRR, with R being a C₁-C₃ alkyl,
B is a ring system selected from the group consisting of 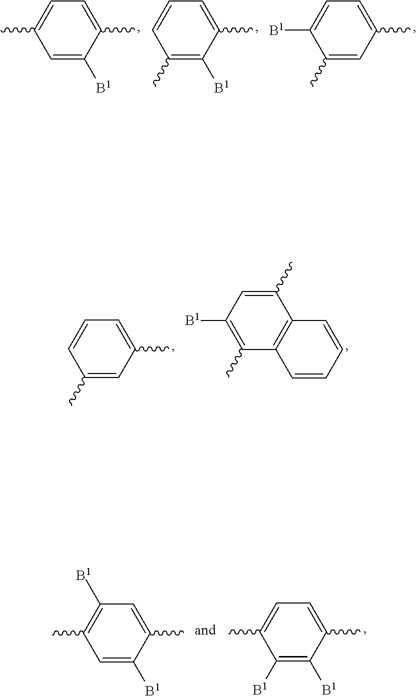
wherein, when B is a phenyl, B¹ is selected from the group consisting of —F, —SCH₃, —SCH₂CH₃, —CF₃, —OCH₃, and —OCF₃ and, when B is a naphtyl, B¹ is —H, and
C is selected from the group consisting of 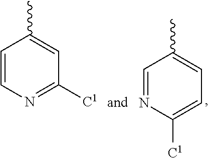
with C¹ being selected from the group consisting of 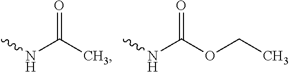
—NH—C(O)OtBu, —NH—C(O)N(CH3)-C(O)OtBu, —NH—C(O)—CH₂—N(CH₃)—C(O)OtBu, —NH—C(O)—CH₂—NHCH₃, —NH—C(O)—Obenzyl, —NH—C(O)—CH₂—OH, preferably 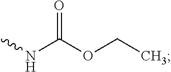
or
wherein A is a ring selected from the group consisting of 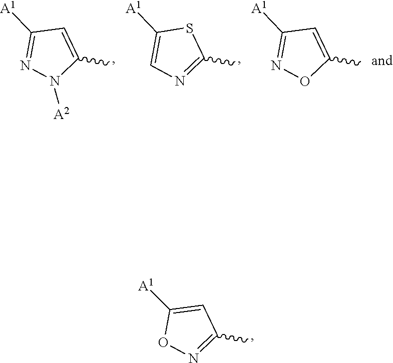
A¹ is tert-butyl,
A² is a phenyl optionally substituted with 1, 2 or 3 substituents selected from the group consisting of methyl, —Cl, —F, —CN, methoxy, —O—(CH₂)₂-piperidinyl, —O—(CH₂)₂-morpholinyl, —O—(CH₂)₃—N(CH₃)₂, and —CH₂-morpholinyl, or A² is 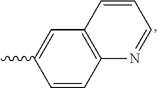
B is a ring system selected in the group consisting of 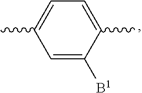
with B¹ being selected from the group consisting of —F, —SCH₃, —SCH₂CH₃, —CF₃, —OCH₃, and —OCF₃, preferably —SCH₃, 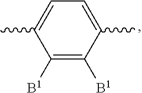
with B¹ being F,
and 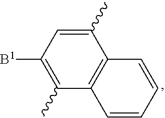
with B¹ being —H, and
C is selected in the group consisting of 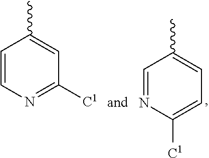
with C¹ being selected from the group consisting of 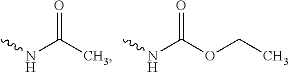
—NH—C(O)OtBu, —NH—C(O)N(CH3)-C(O)OtBu, —NH—C(O)—CH₂—N(CH₃)—C(O)OtBu, —NH—C(O)—CH₂—NHCH₃, —NH—C(O)—Obenzyl, —NH—C(O)—CH₂—OH, preferably

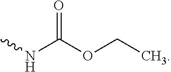

79. A pharmaceutical or veterinary composition comprising a compound as defined in claim 78.
